SEQUENCE LISTING (2 sequences)

SEQ ID NO: 1 - DNA, 2841 bp, unidentified
  source 1..2841: mol_type = genomic DNA
atgcatcaccatcaccatcaccatcacgagaacctgtacttccagggatccatggaaccccgtgatggtagccccgaagctcgtagctccgattccgagtccgccagcgcttcctcctccggtagcgaacgtgacgctggtcccgagcccgacaaagctccccgtcgtctgaataagcgccgttttcccggtctccgtctgttcggccaccgcaaggccatcactaagtccggtctccagcatctggctcctcctcctcctacccccggtgctccttgctccgaatccgagcgccagattcgctccactgtggattggtccgaaagcgccacctatggtgagcatatctggttcgagaccaacgtctccggcgacttctgttatgtcggtgagcaatactgtgtggctcgtatgctgcagaagtccgtgtcccgccgtaaatgcgccgcttgcaaaatcgtggtccataccccttgcatcgagcaactggagaaaatcaacttccgctgcaagcccagctttcgtgagtccggttcccgcaacgtgcgcgaacctactttcgtgcgccaccactgggtgcatcgtcgtcgccaagacggcaaatgccgccactgcggcaaaggttttcagcagaaattcaccttccacagcaaggagatcgtcgccatcagctgcagctggtgcaaacaagcttaccattccaaagtgagctgcttcatgctccagcagatcgaagagccttgctctctgggtgtgcatgctgctgtcgtgattccccctacttggattctgcgtgctcgccgtccccagaacactctgaaggcctccaaaaagaagaagcgcgccagcttcaagcgtaagagctccaaaaagggtcccgaagagggccgttggcgtcccttcatcatccgccctactccttcccctctgatgaagcctctgctggtcttcgtcaaccctaagagcggcggcaaccaaggtgctaaaatcatccagtccttcctctggtatctgaaccctcgtcaagtgttcgacctcagccaaggcggtcctaaggaggctctggagatgtaccgcaaggtccacaatctgcgcatcctcgcttgtggtggcgatggcaccgtgggctggattctgtccactctggaccaactgcgtctgaaacctcccccccccgtggctattctgcctctcggtaccggcaacgatctggctcgtactctgaattggggtggtggctacaccgatgagcccgtgtccaagattctgtcccacgtcgaagaaggcaatgtcgtccaactggaccgttgggacctccacgccgaacccaaccccgaggctggccccgaggaccgtgacgagggcgctactgaccgtctgcccctcgacgtcttcaataattacttctctctgggctttgacgctcacgtgactctggaatttcatgaaagccgcgaggccaaccccgagaagttcaattcccgtttccgcaacaagatgttctacgctggcaccgccttcagcgacttcctcatgggctccagcaaggacctcgctaagcatatccgcgtggtgtgcgatggcatggatctgacccctaagatccaagatctgaagccccaatgtgtcgtgtttctgaacatcccccgctactgcgctggtactatgccttggggccatcccggtgaacaccatgacttcgaacctcagcgtcatgatgacggctatctggaggtgatcggtttcaccatgacctccctcgctgctctgcaagtgggtggccacggcgaacgtctgactcaatgccgcgaggtggtgctgaccaccagcaaagccatccccgtccaagtggatggtgagccttgcaagctggccgcctcccgtatccgtatcgctctccgcaatcaagctaccatggtccagaaggccaaacgccgcagcgctgctcctctccacagcgaccaacaacccgtccccgaacagctgcgcatccaagtgtcccgtgtcagcatgcatgactacgaggctctgcactacgacaaggaacagctgaaggaagccagcgtgcctctgggtactgtggtcgtgcccggtgacagcgatctggagctctgccgtgcccacatcgagcgtctgcagcaagagcccgacggtgctggtgccaagagccctacttgccaaaaactctcccccaagtggtgtttcctcgacgctaccaccgccagccgcttctaccgcattgatcgcgcccaagagcatctgaactatgtcaccgagatcgctcaagacgagatctacatcctcgaccccgaactcctcggtgctagcgcccgtcccgacctccccactcctacctcccctctgcccacttccccttgttcccccacccctcgtagcctccaaggtgatgctgcccctcctcaaggtgaggagctcattgaggccgctaagcgtaacgatttctgcaagctccaagagctgcatcgtgctggtggcgacctcatgcaccgcgatgagcagagccgcactctgctgcaccacgctgtgtccactggtagcaaggacgtggtgcgctatctgctggaccacgctcctcccgagatcctcgacgctgtggaagaaaacggcgagacttgcctccaccaagctgctgctctgggtcaacgtaccatctgccactacatcgtcgaagctggtgcttctctgatgaagaccgaccagcaaggtgatactccccgtcagcgcgccgagaaagcccaagacaccgaactggctgcctatctggagaaccgtcagcactaccagatgattcagcgtgaagaccaagagaccgccgtgtaa

SEQ ID NO: 2 - protein, 929 aa, unidentified
MEPRDGSPEARSSDSESASASSSGSERDAGPEPDKAPRRLNKRRFPGLRLFGHRKAITKSGLQHLAPPPPTPGAPCSESERQIRSTVDWSESATYGEHIWFETNVSGDFCYVGEQYCVARMLQKSVSRRKCAACKIVVHTPCIEQLEKINFRCKPSFRESGSRNVREPTFVRHHWVHRRRQDGKCRHCGKGFQQKFTFHSKEIVAISCSWCKQAYHSKVSCFMLQQIEEPCSLGVHAAVVIPPTWILRARRPQNTLKASKKKKRASFKRKSSKKGPEEGRWRPFIIRPTPSPLMKPLLVFVNPKSGGNQGAKIIQSFLWYLNPRQVFDLSQGGPKEALEMYRKVHNLRILACGGDGTVGWILSTLDQLRLKPPPPVAILPLGTGNDLARTLNWGGGYTDEPVSKILSHVEEGNVVQLDRWDLHAEPNPEAGPEDRDEGATDRLPLDVFNNYFSLGFDAHVTLEFHESREANPEKFNSRFRNKMFYAGTAFSDFLMGSSKDLAKHIRVVCDGMDLTPKIQDLKPQCVVFLNIPRYCAGTMPWGHPGEHHDFEPQRHDDGYLEVIGFTMTSLAALQVGGHGERLTQCREVVLTTSKAIPVQVDGEPCKLAASRIRIALRNQATMVQKAKRRSAAPLHSDQQPVPEQLRIQVSRVSMHDYEALHYDKEQLKEASVPLGTVVVPGDSDLELCRAHIERLQQEPDGAGAKSPTCQKLSPKWCFLDATTASRFYRIDRAQEHLNYVTEIAQDEIYILDPELLGASARPDLPTPTSPLPTSPCSPTPRSLQGDAAPPQGEELIEAAKRNDFCKLQELHRAGGDLMHRDEQSRTLLHHAVSTGSKDVVRYLLDHAPPEILDAVEENGETCLHQAAALGQRTICHYIVEAGASLMKTDQQGDTPRQRAEKAQDTELAAYLENRQHYQMIQREDQETAV

(19) United States
(12) Patent Application Publication
(10) Pub. No.: US 2024/0327417 A1
(43) Pub. Date: Oct. 3, 2024

(54) HETEROCYCLIC COMPOUNDS

(71) Applicant: BeiGene Switzerland GmbH, Basel (CH)

(72) Inventors: Guoliang Zhang, Beijing (CN); Zhikun Ni, Beijing (CN); Jianzhuang Miao, Beijing (CN); Ce Wang, Beijing (CN)

(73) Assignee: Beigene Switzerland GMBH, Basel (CH)

(21) Appl. No.: 18/733,421

(22) Filed: Jun. 4, 2024

Related U.S. Application Data
(63) Continuation of application No. PCT/CN2022/142891, filed on Dec. 28, 2022.

(30) Foreign Application Priority Data
Dec. 29, 2021 (WO) PCT/CN2021/142476

(51) Int. Cl. C07D 487/04; A61K 31/5025; C07D 519/00

(52) U.S. Cl. CPC C07D 487/04; A61K 31/5025; C07D 519/00

(57) ABSTRACT

Disclosed herein is a compound of Formula (I) having the following structure for activating T cells, promoting T cell proliferation, and/or exhibiting antitumor activity, a method of using the compounds disclosed herein for treating cancer, and a pharmaceutical composition comprising the same.

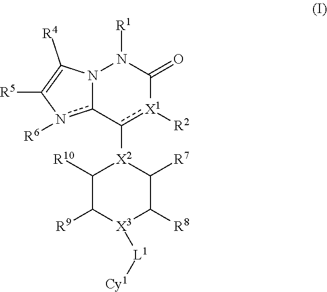

### CROSS-REFERENCE TO RELATED APPLICATIONS

This application is a continuation of International Patent Application No. PCT/CN2022/142891, filed Dec. 28, 2022, which claims priority to International Patent Application No. PCT/CN2021/142476, filed Dec. 29, 2021. The disclosures of each of the aforementioned applications are incorporated herein by reference in their entireties.

### SEQUENCE LISTING

This application contains a Sequence Listing, which has been submitted electronically in XML format. The XML file is entitled “BGB15002-01PCT_Seql.xml,” was created on Apr. 29, 2024, and is 8,192 bytes in size. The Sequence Listing is incorporated herein by reference in its entirety.

### FIELD OF THE DISCLOSURE

Disclosed herein is a compound of Formula (I) for activating T cells, promoting T cell proliferation, and/or exhibiting antitumor activity, a method of using the compounds disclosed herein for treating cancer, and a pharmaceutical composition comprising the same.

### BACKGROUND OF THE DISCLOSURE

Diacylglycerol kinases (DGKs) are a family of lipid kinases that phosphorylates and converts diacylglycerol (DAG) into phosphatidic acid (PA). As the substrate of DGKs, DAG is generated from inositol phospholipids and other phospholipids at the plasma membrane by phospholipase C (PLC) hydrolysis in response to the activation of various cell-surface receptors, including G-protein coupled receptors (GPCR) and immunoreceptor tyrosine-based activation motif (ITAM)-bearing receptors (Rhee, Sue Goo. Annual review of biochemistry. 2001, 70.1: 281-312). DAG is one of the key intracellular second messengers that recruits and activates many downstream effectors including protein kinase C (PKC), protein kinase D (PKD) families, and Ras guanyl nucleotide releasing proteins (RasGRPs), which in turn activates NF-κB and extracellular regulated kinase (ERK) pathways (Mérida, Isabel, et al. Biochemical Journal. 2008, 409.1: 1-18, Joshi, Rohan P., et al. International Journal of Molecular Sciences. 2013, 14.4: 6649-6673). By consuming DAG, DGK controls and tunes the threshold and duration of DAG mediated signaling. Mammalian DGK family comprises 10 different members, in which DGKα, DGKζ and DGKδ are the three major isoforms that are abundantly expressed in lymphoid tissues (Joshi, Rohan P., et al. International Journal of Molecular Sciences. 2013, 14.4: 6649-6673).

Cancer immunotherapy is a type of cancer treatment to manipulate and boost host immune system to recognize and attack cancer cells. A vast majority of studies have focused on targeting immune checkpoint inhibitors, such as CTLA-4 and PD-1/PD-L1, to reinvigorate exhausted CD8⁺ T cells within tumor sites. It was emerged that peripheral T cell tolerance, which under normal circumstances prevents detrimental autoimmune disease, can be hijacked by tumors to prevent anti-tumor immune response during carcinogenesis (Nüssing, Simone, et al. Frontiers in Immunology. 2020, 11: 2461). T cell anergy is one of the most important mechanisms of T cell tolerance and has been reported to occur in tumor infiltrated T cells, which contributes to the immunosuppressive nature of tumor microenvironment (Abe, Brian T., and Fernando Macian. Oncoimmunology. 2013, 2.2: e22679). Anergy-associated transcription factor early growth response gene2 (Egr2) directly binds to Dgka and Dgkz promoter and increases their expression (Zheng, Yan, et al. Journal of Experimental Medicine 2012, 209.12: 2157-2163; Zheng, Yan, et al. Molecular Immunology. 2013, 55.3-4: 283-291). In anergic T cells, both DGKa and DGKζ play critical roles to negatively regulate DAG-signaling downstream of TCR and reduce the strength of TCR activation (Chen, Shelley S., et al. Frontiers in Cell and Developmental Biology. 2016, 4: 130). Thus, immune cell expressed DGKα and DGKζ were investigated as potential targets to reverse the hyporesponsiveness of the tumor infiltrated T cells. It was demonstrated that genetic deletion of DGKα or DGKζ enhanced cytokine production and proliferation of T cells (Olenchock, Benjamin A., et al. Nature immunology. 2006, 7.11: 1174-1181; Zhong, Xiao-Ping, et al. Nature immunology. 2003, 4.9: 882-890). DGKα or DGKζ single knockout in both mouse or human chimeric antigen receptor (CAR)-T cells showed superior effector function as determined by enhanced in vitro cytotoxicity and cytokine secretion when cocultured with antigen expressing titled cells (Riese, Matthew J., et al. Cancer Research. 2013, 73.12: 3566-3577; Jung, In-Young, et al. Cancer Research. 2018, 78.16: 4692-4703). MesoCAR-transduced DGKα or DGKζ deficient T cells also showed significantly elevated in vivo activity against mesotheliomas (Riese, Matthew J., et al. Cancer Research. 2013, 73.12: 3566-3577). DGKζ^{-/-} mice showed enhanced tumor suppressive efficacy with both orthotopic and subcutaneously implanted models (Wesley, Erin M., et al. Immunohorizons. 2018, 2.4: 107-118; Wee, Susan, et al. AACR; Cancer Res 2019; 79(13 Suppl): Abstract nr 936). Besides the T cell regulatory function, both DGKα and DGKζ also involve in tuning NK cell activation at tumor site (Prinz, Petra U., et al. International Journal of Cancer. 2014. 135.8: 1832-1841; Yang, Enjun, et al. The Journal of Immunology. 2016, 197.3: 934-941). In addition, DGKζ were found to play a critical role to control the activation threshold of mature B cells (Wheeler, Matthew L., et al. Science Signaling. 2013, 6.297: ra91-ra91). In summary, all these preclinical data suggest titled inhibition of DGKα and DGKζ could be therapeutically beneficial to promote immunity against cancer.

Although the existing anti-CTLA-4 and anti-PD-1 therapies have shown clear clinical benefits in a subset of patients with various tumor types, there are still unmet medical needs to develop novel immunotherapies to achieve robust and durable clinical anti-tumor efficacy. Pre-clinical data strongly suggests there is a great potential of developing DGKα and DGKζ targeted therapies to improve antitumor immunity.

### SUMMARY OF THE DISCLOSURE

The above needs have been met by providing the compounds disclosed herein which have a novel core structure and show the desired inhibition of DGKα and DGKζ. In some embodiments, the compounds disclosed herein show the dual inhibitory activity of both DGKα and DGKζ. In some embodiments, the compounds disclosed herein show the selective inhibitory activity of DGKα over DGKζ. In some embodiments, the compounds disclosed herein show the selective inhibitory activity of DGKζ over DGKα.

Disclosed herein provides a compound of formula (I),

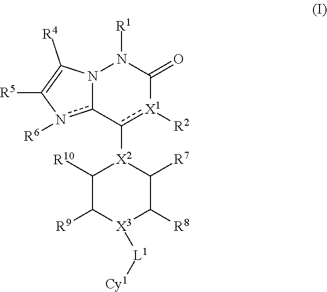

and pharmaceutically acceptable salts, tautomers, stereoisomers, enantiomers, isotopologues, and prodrugs thereof, wherein the variables are defined as herein.

In some embodiments, the compound of formula (I) is a compound of formula (II):

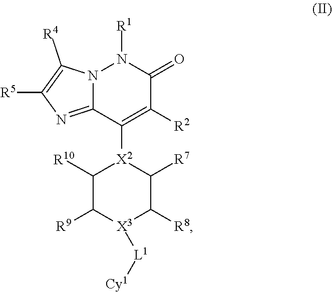

wherein the variables are defined as herein.

In some embodiments, the compound of formula (II) is a compound of formula (III):

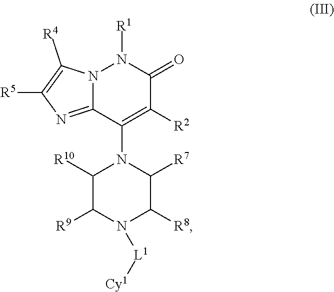

wherein the variables are defined as herein.

In some embodiments, the compound of formula (I) is a compound of formula (IV):

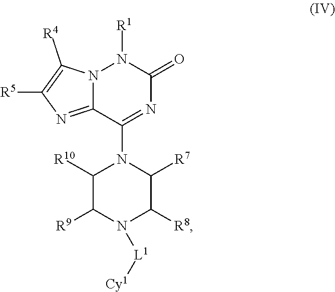

wherein the variables are defined as herein.

In some embodiments, the compound of formula (I) is a compound of formula (V):

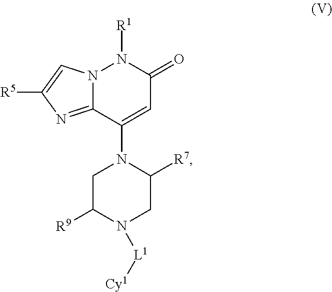

wherein the variables are defined as herein.

In some embodiments, the compound of formula (I) is a compound of formula (VI):

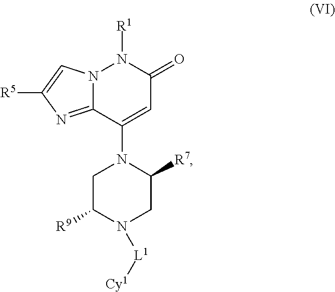

wherein the variables are defined as herein.

In one aspect, provided herein are methods for inhibiting a kinase, for example, DGKα, DGKζ, or both, in a cell expressing said kinase, comprising contacting said cell with an effective amount of a compound as described herein. In one aspect, provided herein are methods for treating diseases of mammals, including humans, in particular hyperproliferative disorders, such as cancer.

In another aspect provided herein are methods for preparing compounds as described herein.

The present embodiments can be understood more fully by reference to the detailed description and examples, which are intended to exemplify non-limiting embodiments.

### DETAILED DESCRIPTION OF THE DISCLOSURE

### Definitions

The following terms have the indicated meanings throughout the specification:

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The term “alkyl” refers to a hydrocarbon group selected from linear and branched saturated hydrocarbon groups derived from an alkane by removal of one hydrogen atom from the same carbon atom, which comprises from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl ort-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups. The alkyl group can be optionally enriched in deuterium, e.g., —CD₃, —CD₂CD₃ and the like. The term “alkylene” refers to a hydrocarbon group selected from linear and branched saturated hydrocarbon groups derived from an alkane by removal of two hydrogen atoms from the same carbon atom, which comprises from 1 to 6, such as from 1 to 4, carbon atoms, further such as from 1 to 3, more further such as 1, 2 or 3 carbon atoms, include, but not limited to, methylene (—CH₂—), ethylene (—CH₂CH₂—), 1-methymethylene (—CH(CH₃)—), or trimethylene (—CH₂CH₂CH₂—). When the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, O(alkyl)aminocarbonyl, aryl, heterocyclyl, or heteroaryl.

The term “halogen” refers to fluoro (F), chloro (Cl), bromo (Br) and iodo (I).

The term “haloalkyl” refers to an alkyl group in which one or more hydrogen is/are replaced by one or more halogen atoms such as fluoro, chloro, bromo, and iodo. Examples of the haloalkyl include haloC₁₋₈alkyl, haloC₁₋₆alkyl or halo C₁₋₄alkyl, but not limited to —CF₃, —CH₂Cl, —CH₂CF₃, —CCl₂, CF₃, and the like.

The term “alkyloxy” or “alkoxy” refers to an alkyl group as defined above attached to the parent molecular moiety through an oxygen atom. Examples of an alkyloxy, e.g., C₁₋₆alkyloxy or C₁₋₄ alkyloxy include, but not limited to, methoxy, ethoxy, isopropoxy, propoxy, n-butoxy, tert-butoxy, pentoxy and hexoxy and the like.

The term “amino” refers to —NH₂.

The term “alkenyl” herein refers to a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkynyl” herein refers to a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C2-6 alkynyl, include, but not limited to ethynyl, 1-propynyl, 2-propynyl (propargyl), 1-butynyl, 2-butynyl, and 3-butynyl groups.

The term “cycloalkyl” refers to a hydrocarbon group selected from saturated cyclic hydrocarbon groups, comprising monocyclic and polycyclic (e.g., bicyclic and tricyclic) groups including fused, bridged or spiro cycloalkyl. For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. In particular, Examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embedment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems.

The term “deuterated” is used herein to modify a chemical structure or an organic group or radical, wherein one or more carbon-bound hydrogen(s) are replaced by one or more deuterium(s), e.g., “deuterated-alkyl”, “deuterated-cycloalkyl”, “deuterated-heterocycloalkyl”, “deuterated-aryl”, “deuterated-morpholinyl”, and the like. For example, the term “deuterated-alkyl” defined above refers to an alkyl group as defined herein, wherein at least one hydrogen atom bound to carbon is replaced by a deuterium. In a deuterated alkyl group, at least one carbon atom is bound to a deuterium; and it is possible for a carbon atom to be bound to more than one deuterium; it is also possible that more than one carbon atom in the alkyl group is bound to a deuterium.

The term “aryl” used alone or in combination with other terms refers to an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like). In some embodiments, an aryl group refers to a group selected from: 5- and 6-membered carbocyclic aromatic rings, e.g., phenyl; bicyclic ring systems such as 7 to 12-membered bicyclic ring systems, wherein at least one ring is carbocyclic and aromatic, e.g., naphthyl and indanyl; and, tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeably throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

The term “heteroaryl” herein refers to a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from nitrogen, oxygen or optionally oxidized sulfur as ring member(s), with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from nitrogen, oxygen or optionally oxidized sulfur as ring member(s), with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

The term “optionally oxidized sulfur” used herein refers to —S—, SO or SO₂.

The terms “aromatic heterocyclic ring” and “heteroaryl” are used interchangeably throughout the disclosure herein. In some embodiments, a monocyclic or bicyclic aromatic heterocyclic ring has 5-, 6-, 7-, 8-, 9- or 10-ring forming members with 1, 2, 3, or 4 heteroatom ring members independently selected from nitrogen (N), sulfur (S) and oxygen (O) and the remaining ring members being carbon. In some embodiments, the monocyclic or bicyclic aromatic heterocyclic ring is a monocyclic or bicyclic ring comprising 1 or 2 heteroatom ring members independently selected from nitrogen (N), sulfur (S) and oxygen (O). In some embodiments, the monocyclic or bicyclic aromatic heterocyclic ring is a 5- to 6-membered heteroaryl ring, which is monocyclic and which has 1 or 2 heteroatom ring members independently selected from nitrogen (N), sulfur (S) and oxygen (O). In some embodiments, the monocyclic or bicyclic aromatic heterocyclic ring is a 8- to 10-membered heteroaryl ring, which is bicyclic and which has 1 or 2 heteroatom ring members independently selected from nitrogen, sulfur and oxygen.

Examples of the heteroaryl group or the monocyclic or bicyclic aromatic heterocyclic ring include, but are not limited to, (as numbered from the linkage position assigned priority 1) 1H-pyrazolyl (such as 1H-pyrazol-3-yl, 1H-pyrazol-4-yl or 1H-pyrazol-5-yl), pyridyl or pyridinyl (such as 2-pyridyl, 3-pyridyl, or 4-pyridyl), cinnolinyl, pyrazinyl, pyrimidinyl (such as pyrimidin-2-yl, pyrimidin-4-yl, pyrimidin-5-yl or 2,4-pyrimidinyl, 3,5-pyrimidinyl), imidazolyl (such as 1H-imidazol-2-yl, 1H-imidazol-4-yl, 1H-imidazol-5-yl, or 2,4-imidazolyl), imidazopyridinyl, isoxazolyl, oxazolyl, thiazolyl, isothiazolyl, thiadiazolyl (such as 1,2,3-thiadiazolyl, 1,2,4-thiadiazolyl, or 1,3,4-thiadiazolyl), tetrazolyl, thienyl (such as thien-2-yl, thien-3-yl), triazinyl, benzothienyl, furyl or furanyl, benzofuryl, benzoimidazolyl, indolyl (such as 1H-indol-2-yl, 1H-indol-3-yl, 1H-indol-4-yl, 1H-indol-5-yl, 1H-indol-6-yl or 1H-indol-7-yl), isoindolyl, indolinyl, oxadiazolyl (such as 1,2,3-oxadiazolyl, 1,2,4-oxadiazolyl, or 1,3,4-oxadiazolyl), phthalazinyl, pyrazinyl (such as pyrazin-2-yl), pyridazinyl, pyrrolyl, triazolyl (such as 1,2,3-triazolyl, 1,2,4-triazolyl, or 1,3,4-triazolyl), quinolinyl (such as quinolin-2-yl, quinolin-3-yl, quinolin-4-yl, quinolin-5-yl, quinolin-6-yl, or quinolin-7-yl), isoquinolinyl (such as isoquinolin-1-yl, isoquinolin-3-yl, isoquinolin-4-yl, isoquinolin-5-yl, isoquinolin-6-yl, isoquinolin-7-yl, or isoquinolin-8-yl), pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl (such as furazan-2-yl, furazan-3-yl), benzofurazanyl, benzothiophenyl, benzothiazolyl, benzoxazolyl (such as benzo[d]oxazol-2-yl, benzo[d]oxazol-4-yl, benzo[d]oxazol-5-yl, benzo[d]oxazol-6-yl or benzo[d]oxazol-7-yl), quinazolinyl, quinoxalinyl (such as quinoxalin-2-yl, quinoxalin-3-yl, quinoxalin-4-yl, quinoxalin-5-yl, quinoxalin-6-yl, quinoxalin-7-yl or quinoxalin-8-yl), naphthyridinyl (such as 1,8-naphthyridin-2-yl, 1,8-naphthyridin-3-yl, or 1,8-naphthyridin-4-yl), 2,3-dihydro-[1,4]dioxino[2,3-b]pyridinyl (such as 2,3-dihydro-[1,4]dioxino[2,3-b]pyridin-6-yl, 2,3-dihydro-[1,4]dioxino[2,3-b]pyridin-7-yl, or 2,3-dihydro-[1,4]dioxino[2,3-b]pyridin-8-yl), furopyridinyl, benzothiazolyl (such as benzo[d]thiazol-2-yl, benzo[d]thiazol-4-yl, benzo[d]thiazol-5-yl, benzo[d]thiazol-6-yl or benzo[d]thiazol-7-yl), benzo[d]imidazolyl (such as 1H-benzo[d]imidazol-2-yl, 1H-benzo[d]imidazol-4-yl, 1H-benzo[d]imidazol-5-yl, 1H-benzo[d]imidazol-6-yl or 1H-benzo[d]imidazol-7-yl), [1,2,4]triazolo[1,5-a]pyridinyl (such as [1,2,4]triazolo[1,5-a]pyridin-2-yl, [1,2,4]triazolo[1,5-a]pyridin-5-yl, [1,2,4]triazolo[1,5-a]pyridin-6-yl, [1,2,4]triazolo[1,5-a]pyridin-7-yl, or [1,2,4]triazolo[1,5-a]pyridin-8-yl), 3H-imidazo[4,5-b]pyridinyl (such as 3H-imidazo[4,5-b]pyridin-2-yl, 3H-imidazo[4,5-b]pyridin-5-yl, 3H-imidazo[4,5-b]pyridin-6-yl or 3H-imidazo[4,5-b]pyridin-7-yl), 1H-imidazo[4,5-b]pyridinyl (such as 1H-imidazo[4,5-b]pyridin-2-yl, 1H-imidazo[4,5-b]pyridin-5-yl, 1H-imidazo[4,5-b]pyridin-6-yl, 1H-imidazo[4,5-b]pyridin-7-yl), [1,2,4]triazolo[1,5-a]pyridinyl (such as [1,2,4]triazolo[1,5-a]pyridin-2-yl, [1,2,4]triazolo[1,5-a]pyridin-5-yl, [1,2,4]triazolo[1,5-a]pyridin-6-yl, [1,2,4]triazolo[1,5-a]pyridin-7-yl or [1,2,4]triazolo[1,5-a]pyridin-8-yl), indazolyl (such as 1H-indazol-5-yl), 5,6,7,8-tetrahydroisoquinoline, thiazolo[5,4-b]pyridinyl (such as thiazolo[5,4-b]pyridin-2-yl, thiazolo[5,4-b]pyridin-5-yl, thiazolo[5,4-b]pyridin-6-yl or thiazolo[5,4-b]pyridin-7-yl), thiazolo[4,5-b]pyridinyl (such as thiazolo[4,5-b]pyridin-2-yl, thiazolo[4,5-b]pyridin-5-yl, thiazolo[4,5-b]pyridin-6-yl orthiazolo[4,5-b]pyridin-7-yl), thieno[2,3-b]pyridinyl (such as thieno[2,3-b]pyridin-2-yl, thieno[2,3-b]pyridin-3-yl, thieno[2,3-b]pyridin-4-yl, thieno[2,3-b]pyridin-5-yl or thieno[2,3-b]pyridin-6-yl), thieno[3,2-b]pyridinyl (such as thieno[3,2-b]pyridin-2-yl, thieno[3,2-b]pyridin-3-yl, thieno[3,2-b]pyridin-5-yl, thieno[3,2-b]pyridin-6-yl, or thieno[3,2-b]pyridin-7-yl).

Also, a “heteroaryl” fused with a “Heterocyclyl” is defined as “heteroaryl”.

“Heterocyclyl,” “heterocycle” or “heterocyclic” are interchangeable and refer to a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “monocyclic heterocyclyl” refers to monocyclic groups in which at least one ring member is a heteroatom selected from nitrogen, oxygen or optionally oxidized sulfur. A heterocycle may be saturated or partially saturated.

Exemplary monocyclic 4 to 9-membered heterocyclyl groups include, but not limited to, (as numbered from the linkage position assigned priority 1) pyrrolidin-1-yl, pyrrolidin-2-yl, pyrrolidin-3-yl, imidazolidin-2-yl, imidazolidin-4-yl, pyrazolidin-2-yl, pyrazolidin-3-yl, piperidin-1-yl, piperidin-2-yl, piperidin-3-yl, piperidin-4-yl, 2,5-piperazinyl, pyranyl, morpholinyl, morpholino, morpholin-2-yl, morpholin-3-yl, oxiranyl, aziridin-1-yl, aziridin-2-yl, azocan-1-yl, azocan-2-yl, azocan-3-yl, azocan-4-yl, azocan-5-yl, thiiranyl, azetidin-1-yl, azetidin-2-yl, azetidin-3-yl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepan-1-yl, azepan-2-yl, azepan-3-yl, azepan-4-yl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepanyl, 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinyl, imidazolinyl, pyrimidinonyl, or 1,1-dioxo-thiomorpholinyl.

The term “spiro heterocyclyl” refers to a 5 to 20-membered polycyclic heterocyclyl with rings connected through one common carbon atom (called a spiro atom), comprising one or more heteroatoms selected from nitrogen, oxygen or optionally oxidized sulfur as ring members, with the remaining ring members being carbon. One or more rings of a spiro heterocyclyl group may contain one or more double bonds, but none of the rings has a completely conjugated pi-electron system. Preferably a spiro heterocyclyl is 6 to 14-membered, and more preferably 7 to 12-membered. According to the number of common spiro atoms, a spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, and more preferably 4-membered/4-membered, 3-membered/5-membered, 4-membered/5-membered, 4-membered/6-membered, 5-membered/5-membered, or 5-membered/6-membered mono-spiro heterocyclyl.

The term “fused heterocyclic group” refers to a 5 to 20-membered polycyclic heterocyclyl group, wherein each ring in the system shares an adjacent pair of atoms (carbon and carbon atoms or carbon and nitrogen atoms) with another ring, comprising one or more heteroatoms selected from nitrogen, oxygen or optionally oxidized sulfur as ring members, with the remaining ring members being carbon. One or more rings of a fused heterocyclic group may contain one or more double bonds, but none of the rings has a completely conjugated pi-electron system. Preferably, a fused heterocyclyl is 6 to 14-membered, and more preferably 7 to 10-membered. According to the number of membered rings, a fused heterocyclyl is divided into bicyclic, tricyclic, tetracyclic, or polycyclic fused heterocyclyl, preferably refers to bicyclic or tricyclic fused heterocyclyl, and more preferably 5-membered/5-membered, or 5-membered/6-membered bicyclic fused heterocyclyl. Representative examples of fused heterocycles include, but not limited to, the following groups octahydrocyclopenta[c]pyrrole (e.g., octahydrocyclopenta[c]pyrrol-2-yl), octahydropyrrolo[3,4-c]pyrrolyl, octahydroisoindolyl, isoindolinyl (e.g., isoindoline-2-yl), octahydro-benzo[b][1,4]dioxin.

The term “bridged heterocyclyl” refers to a 5- to 14-membered polycyclic heterocyclic alkyl group, wherein every two rings in the system share two disconnected atoms, comprising one or more heteroatoms selected from nitrogen, oxygen or optionally oxidized sulfur as ring members, with the remaining ring members being carbon. One or more rings of a bridged heterocyclyl group may contain one or more double bonds, but none of the rings has a completely conjugated pi-electron system. Preferably, a bridged heterocyclyl is 6 to 14-membered, and more preferably 7 to 10-membered. According to the number of membered rings, a bridged heterocyclyl is divided into bicyclic, tricyclic, tetracyclic or polycyclic bridged heterocyclyl, and preferably refers to bicyclic, tricyclic or tetracyclic bridged heterocyclyl, and more preferably bicyclic or tricyclic bridged heterocyclyl. Representative examples of bridged heterocyclyls include, but not limited to, the following groups: 2-azabicyclo[2.2.1]heptyl, azabicyclo[3.1.0]hexyl, 2-azabicyclo[2.2.2]octyl and 2-azabicyclo[3.3.2]decyl.

A “cycloalkylalkyl” group is a radical of the formula: -alkyl-cycloalkyl, wherein alkyl and cycloalkyl are as defined above. Substituted cycloalkylalkyl groups may be substituted at the alkyl, the cycloalkyl, or both the alkyl and the cycloalkyl portions of the group. Representative cycloalkylalkyl groups include but are not limited to methylcyclopropyl, methylcyclobutyl, methylcyclopentyl, methylcyclohexyl, ethylcyclopropyl, ethylcyclobutyl, ethylcyclopentyl, ethylcyclohexyl, propylcyclopentyl, propylcyclohexyl and the like.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

An “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocylylalkyl groups include but are not limited to 4-ethyl-morpholinyl, 4-propylmorpholinyl, furan-2-yl methyl, furan-3-yl methyl, pyridin-3-yl methyl, tetrahydrofuran-2-yl ethyl, and indol-2-yl propyl.

A “halogen” is fluorine, chlorine, bromine or iodine.

A “hydroxyalkyl” group is an alkyl group as described above substituted with one or more hydroxy groups.

An “alkoxy” group is —O-(alkyl), wherein alkyl is defined above.

An “alkoxyalkyl” group is -(alkyl)-O-(alkyl), wherein alkyl is defined above.

An “amino” group is a radical of the formula: —NH₂.

An “alkylamino” group is a radical of the formula: —NH-alkyl or —N(alkyl)₂, wherein each alkyl is independently as defined above.

A “carboxy” group is a radical of the formula: —C(O)OH.

An “aminocarbonyl” group is a radical of the formula: —C(O)N(R^{#})₂, —C(O)NH(R^{#}) or —C(O)NH₂, wherein each R^{#}is independently a substituted or unsubstituted alkyl, cycloalkyl, aryl, aralkyl, heterocyclyl or heterocyclyl group as defined herein.

An “acylamino” group is a radical of the formula: —NHC(O)(R^{#}) or —N(alkyl)C(O)(R^{#}), wherein each alkyl and R^{#}are independently as defined above.

A “sulfonylamino” group is a radical of the formula: —NHSO₂(R^{#}) or —N(alkyl)SO₂(R^{#}), wherein each alkyl and R^{#}are defined above.

A “urea” group is a radical of the formula: —N(alkyl)C(O)N(R^{#})₂, —N(alkyl)C(O)NH(R^{#}), —N(alkyl)C(O)NH₂, —NHC(O)N(R^{#})₂, —NHC(O)NH(R^{#}), or —NH(CO)NHR^{#}, wherein each alkyl and R^{#}are independently as defined above.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

The term “substantially pure” as used herein means that the titled stereoisomer contains no more than 35%, such as no more than 30%, further such as no more than 25%, even further such as no more than 20%, by weight of any other stereoisomer(s). In some embodiments, the term “substantially pure” means that the titled stereoisomer contains no more than 10%, for example, no more than 5%, such as no more than 1%, by weight of any other stereoisomer(s).

When compounds disclosed herein contain olefinic double bonds, unless specified otherwise, such double bonds are meant to include both E and Z geometric isomers.

When compounds disclosed herein contain a di-substituted cyclohexyl or cyclobutyl group, substituents found on cyclohexyl or cyclobutyl ring may adopt cis and trans formations. Cis formation means that both substituents are found on the upper side of the 2 substituent placements on the carbon, while trans would mean that they were on opposing sides.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or flash column chromatography. Flash column chromatography can involve any number of methods including, for example: reverse-phase and normal phase; size exclusion; ion exchange; high, medium and low pressure liquid flash column chromatography methods and apparatus; small scale analytical; simulated moving bed (“SMB”) and preparative thin or thick layer flash column chromatography, as well as techniques of small scale thin layer and flash column flash column chromatography. One skilled in the art will apply techniques most likely to achieve the desired separation.

“Diastereomers” refers to stereoisomers of a compound with two or more chiral centers but which are not mirror images of one another. Diastereomeric mixtures can be separated into their individual diastereomers on the basis of their physical chemical differences by methods well known to those skilled in the art, such as by flash column chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents [Eliel, E. and Wilen, S. Stereochemistry of Organic Compounds. New York: John Wiley & Sons, Inc., 1994; Lochmuller, C. H, et al. “Flash column chromatographyic resolution of enantiomers: Selective review.” J. Chromatogr., 113(3) (1975): pp. 283-302]. Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

“Pharmaceutically acceptable salts” refers to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

“Selective inhibitory activity” or “selectivity” refers to the difference in the degree of inhibition against DGKα and DGKζ; In some embodiments, “a compound showing selective inhibitory activity of DGKα over DGKζ” refers a compound which shows a ratio of IC50 against DGKζ and IC50 against DGKα larger than or equal to about 20. In some embodiments, “a compound showing selective inhibitory activity of DGKζ over DGKα” refers a compound which shows a ratio of IC50 against DGKα and IC50 against DGKζ larger than or equal to about 20. In some embodiments, “a compound showing selective inhibitory activity of DGKα over DGKζ” refers a compound which shows an IC50 against DGKα is not larger than about 2000 nM with the ratio of IC50 against DGKζ and IC50 against DGKα larger than or equal to about 20; “a compound showing selective inhibitory activity of DGKζ over DGKα” refers a compound which shows an IC50 against DGKζ is not larger than about 2000 nM with the ratio of IC50 against DGKα and IC50 against DGKζ larger than or equal to about 20; and “a compound showing dual inhibitory activity” refers to a compound which shows inhibitory activities against both DGKα and DGKζ with IC50 no larger than 500 nM and the ratio of the two IC50 values no more than 20. In some embodiments, “a compound showing dual inhibitory activity” refers to a compound which shows inhibitory activities against both DGKα and DGKζ with IC50 no larger than 1000 nM and the ratio of the two IC50 values no more than 20. In some embodiments, “a compound showing dual inhibitory activity” refers to a compound which shows inhibitory activities against both DGKα and DGKζ with IC50 no larger than 2000 nM and the ratio of the two IC50 values no more than 20.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” include salts of at least one compound of Formula (I), and salts of the stereoisomers of the compound of Formula (I), such as salts of enantiomers, and/or salts of diastereomers.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer thereof, and/or at least one pharmaceutically acceptable salt thereof disclosed herein effective to “treat” as defined above, a disease or disorder in a subject. In the case of combination therapy, the “therapeutically effective amount” refers to the total amount of the combination objects for the effective treatment of a disease, a disorder or a condition.

The pharmaceutical composition comprising the compound disclosed herein can be administrated via oral, inhalation, rectal, parenteral or topical administration to a subject in need thereof. For oral administration, the pharmaceutical composition may be a regular solid Formulation such as tablets, powder, granule, capsules and the like, a liquid Formulation such as water or oil suspension or other liquid Formulation such as syrup, solution, suspension or the like; for parenteral administration, the pharmaceutical composition may be solution, water solution, oil suspension concentrate, lyophilized powder or the like. Preferably, the Formulation of the pharmaceutical composition is selected from tablet, coated tablet, capsule, suppository, nasal spray or injection, more preferably tablet or capsule. The pharmaceutical composition can be a single unit administration with an accurate dosage. In addition, the pharmaceutical composition may further comprise additional active ingredients.

All Formulations of the pharmaceutical composition disclosed herein can be produced by the conventional methods in the pharmaceutical field. For example, the active ingredient can be mixed with one or more excipients, then to make the desired Formulation. The “pharmaceutically acceptable excipient” refers to conventional pharmaceutical carriers suitable for the desired pharmaceutical Formulation, for example: a diluent, a vehicle such as water, various organic solvents, etc., a filler such as starch, sucrose, etc. a binder such as cellulose derivatives, alginates, gelatin and polyvinylpyrrolidone (PVP); a wetting agent such as glycerol; a disintegrating agent such as agar, calcium carbonate and sodium bicarbonate; an absorption enhancer such as quaternary ammonium compound; a surfactant such as hexadecanol; an absorption carrier such as Kaolin and soap clay; a lubricant such as talc, calcium stearate, magnesium stearate, polyethylene glycol, etc. In addition, the pharmaceutical composition further comprises other pharmaceutically acceptable excipients such as a decentralized agent, a stabilizer, a thickener, a complexing agent, a buffering agent, a permeation enhancer, a polymer, aromatics, a sweetener, and a dye.

The term “disease” refers to any disease, discomfort, illness, symptoms or indications, and can be interchangeable with the term “disorder” or “condition”.

Throughout this specification and the claims which follow, unless the context requires otherwise, the term “comprise,” and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cn-m” indicates a range which includes the endpoints, wherein n and m are integers and indicate the number of carbons. Examples include C₁₋₈, C₁₋₆, and the like.

The term “Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

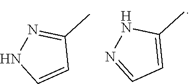

As readily understood by one skilled in the art, a wide variety of functional groups and other structures may exhibit tautomerism and all tautomers of compounds of formula (I) are within the scope of the present invention.

Unless specifically defined elsewhere in this document, all other technical and scientific terms used herein have the meaning commonly understood by one of ordinary skill in the art to which this invention belongs.

It should also be noted the compounds provided herein can contain unnatural proportions of atomic isotopes at one or more of the atoms. For example, the compounds may be radiolabeled with radioactive isotopes, such as for example tritium (H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically encriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

The term “DGK” or “DAGK” (Diacylglycerol kinase) refers to a family of enzymes that catalyzes the conversion of diacylglycerol (DAG) to phosphatidic acid (PA), utilizing ATP as a source of the phosphate. In non-stimulated cells, DGK activity is low, allowing DAG to be used for glycerophospholipid biosynthesis, but on receptor activation of the phosphoinositide pathway, DGK activity increases, driving the conversion of DAG to PA. As both lipids are thought to function as bioactive lipid signaling molecules with distinct cellular targets, DGK therefore occupies an important position, effectively serving as a switch by terminating the signalling of one lipid while simultaneously activating signalling by another. See Mérida I, Avila-Flores A, Merino E (January 2008). “Diacylglycerol kinases: at the hub of cell signalling,” The Biochemical Journal, 409 (1): 1-18. Currently, ten members of the DGK family have been cloned and identified. Although all family members have conserved catalytic domains and two cysteine rich domains, they are further classified into five groups according to the presence of additional functional domains and substrate specificity. See van Blitterswijk W J, Houssa B (October 2000), “Properties and functions of diacylglycerol kinases,” Cellular Signalling, 12 (9-10): 595-605. These are as follows:
Type 1—DGK-α, DGK-μ, DGK-γ—contain EF-hand motifs and a recoverin homology domain
Type 2—DGK-δ, DGK-η, DGK-κ—contain a pleckstrin homology domain
Type 3—DGK-ε—has specificity for arachidonate-containing DAG
Type 4—DGK-ζ, DGK-t—contain a MARCKS homology domain, ankyrin repeats, a C-terminal nuclear localisation signal, and a PDZ-binding motif.
Type 5—DGK-θ—contains a third cysteine-rich domain, a pleckstrin homology domain and a proline rich region.

The term “treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, “treating” means and alleviation, in whole or in part, of a disorder, disease or condition, or symptoms associated with a condition, treatable or preventable by inhibition of a DGK pathway. In another embodiment, “treating” means and alleviation, in whole or in part, of a disorder, disease or condition, or symptoms associated with a condition, treatable or preventable by inhibition of a DGK-α pathway. In another embodiment, “treating” means and alleviation, in whole or in part, of a disorder, disease or condition, or symptoms associated with a condition, treatable or preventable by inhibition of a DGK-ζ pathway. In one embodiment, the disorder is cancer.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In another, the disorder is a condition, treatable or preventable by inhibition of a DGK pathway. In another, the disorder is a condition, treatable or preventable by inhibition of a DGK-α pathway. In another, the disorder is a condition, treatable or preventable by inhibition of a DGK-ζ pathway. In one embodiment, the disorder is cancer.

The term “subject” includes an animal, including, but not limited to, an animal such a cow, monkey, horse, sheep, pig, chicken, turkey, quail, cat, dog, mouse, rat, rabbit or guinea pig, in one embodiment a mammal, in another embodiment a human. In one embodiment, a subject is a human having or at risk for having liver fibrotic disorders or diabetes or metabolic syndrome leading to liver fibrotic disorders, or a condition, treatable or preventable by inhibition of a JNK pathway, or a symptom thereof.

As used herein, the term “Compound” refers to compounds of formula (I) as well as to further embodiments provided herein. In one embodiment, a “Compound” is a compound set forth in Table 1. The term “Compound” includes pharmaceutically acceptable salts, tautomers, isotopologues, stereoisomers, and prodrugs of the compounds provided herein.

As used herein and unless otherwise indicated, the term “prodrug” means a compound that can hydrolyze, oxidize, or otherwise react under biological conditions (in vitro or in vivo) to provide an active compound, particularly a compound of formula (I). Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a Compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof.

### Compounds

The compounds provided herein have a novel core structure and show the desired inhibition of DGKα and DGKζ. In some embodiments, the compounds disclosed herein show the dual inhibitory activity of both DGKα and DGKζ. In some embodiments, the compounds disclosed herein show the selective inhibitory activity of DGKα over DGKζ. In some embodiments, the compounds disclosed herein show the selective inhibitory activity of DGKζ over DGKα.

### Aspect 1

Disclosed herein provides a compound of formula (I),

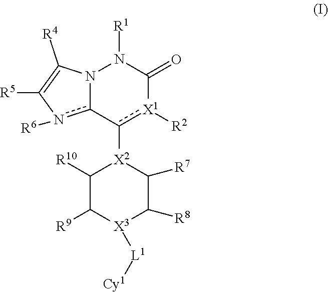

or a pharmaceutically acceptable salt, a tautomer, a stereoisomer, an enantiomer, an isotopologue, or a prodrug thereof,
wherein
X¹ is C or N,
each of X² and X³ is independently selected from —N—or —CH—;
the symbol [symbol] is a single or double bond,
R¹ is hydrogen, deuterium, halogen, substituted or unsubstituted alkyl, substituted or unsubstituted alkoxyl, substituted or unsubstituted cycloalkyl, substituted or unsubstituted aryl, substituted or unsubstituted heterocyclyl, or substituted or unsubstituted heteroaryl;
R² is hydrogen, halogen, cyano, substituted or unsubstituted alkyl, substituted or unsubstituted alkoxyl, substituted or unsubstituted cycloalkyl, substituted or unsubstituted aryl, substituted or unsubstituted heterocyclyl, or substituted or unsubstituted heteroaryl, provided that R² is absent when X¹ is N and the bond [symbol] attached to X¹ is a double bond;
R⁴ is hydrogen, halogen, cyano, substituted or unsubstituted alkyl, substituted or unsubstituted alkoxyl, substituted or unsubstituted cycloalkyl, substituted or unsubstituted aryl, substituted or unsubstituted heterocyclyl, or substituted or unsubstituted heteroaryl;
R⁵ is hydrogen, halogen, cyano, substituted or unsubstituted alkyl, substituted or unsubstituted alkoxyl, substituted or unsubstituted alkenyl, substituted or unsubstituted alkynyl, substituted or unsubstituted cycloalkyl, substituted or unsubstituted aryl, substituted or unsubstituted heterocyclyl, or substituted or unsubstituted heteroaryl, R^{5a}—C(O)—, R^{5a}—C(O)O—, R^{5a}—O—C(O)—, R^{5a}—C(O)NR^{5b}—, R^{5a}—NR^{5b}—C(O)—, or R^{5a}—SO₂—wherein R^{5a} and R^{5b} are each independently hydrogen, alkyl, or cycloalkyl;
R⁶ is absent, hydrogen, halogen, or alkyl which is unsubstituted or substituted with halogen or cyano, provided that R⁶ is absent when the bond [symbol] attached to the nitrogen to which R⁶ is attached is a double bond;
each of R⁷, R⁹, R⁸, and R¹⁰ is independently hydrogen, cyano, substituted or unsubstituted alkyl, substituted or unsubstituted alkoxyl, substituted or unsubstituted cycloalkyl, —C(O)R^{7a}, or -alkyl-C(O)R^{7a}, and wherein R^{7a} is hydrogen, alkyl, or alkoxy, provided that at least one of R⁷ and R⁸ is not hydrogen;
or R⁷ and R⁹ are each hydrogen and R⁸ and R¹⁰ together form a bridge containing at least one —CH₂-moiety in addition to the two bridgehead atoms; or R⁸ and R¹⁰ are each hydrogen and R⁷ and R⁹ together form a bridge containing at least one —CH₂— moiety in addition to the two bridgehead atoms;
L¹ is a direct bond, —O—, —N(R^{L})—, substituted or unsubstituted alkyl, -alkylene or —C(O)—, wherein R^{L} is hydrogen or alkyl;
Cy¹ is unsubstituted or substituted cycloalkyl, unsubstituted or substituted aryl, unsubstituted or substituted heterocyclyl, or unsubstituted or substituted heteroaryl, wherein Cy¹ is optionally substituted with one, two or three substituents R^{3a}.
   wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, cycloalkyl or heterocyclyl;
   optionally wherein two R^{3a} connect to the same carbon and together form a spirocyclic ring; optionally wherein two R^{3a} form a fused ring with Cy¹, wherein R^{3a} is unsubstituted or substituted with cyano, alkoxy, alkyl, halogen, or hydroxy; and
   wherein R^{3b} and R^{3c} are each independently hydrogen or alkyl.

In some embodiments, the compound of formula (I) is a compound of formula (II):

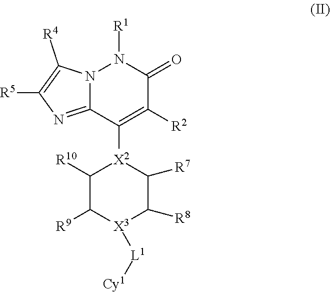

wherein the variables are defined as herein.

In some embodiments, the compound of formula (II) is a compound of formula (III):

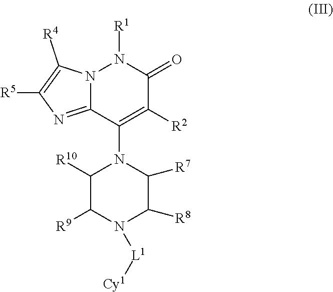

wherein the variables are defined as herein.

In some embodiments, the compound of formula (I) is a compound of formula (IV):

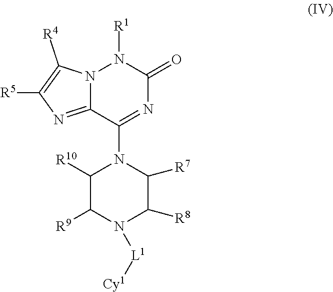

wherein the variables are defined as herein.

The definitions of R¹

In some embodiments, R¹ is hydrogen, or substituted or unsubstituted alkyl. In some embodiments, R¹ is hydrogen, or substituted or unsubstituted C₁₋₄alkyl.

In some embodiments, R¹ is hydrogen, or C₁₋₄alkyl optionally substituted with deuterium, halogen, hydroxy, alkoxy or cycloalkyl. In some embodiments, R¹ is hydrogen, or C₁₋₄alkyl optionally substituted with deuterium, halogen, hydroxy, alkoxy or cycloalkyl. In some embodiments, R¹ is hydrogen, or C₁₋₃alkyl optionally substituted with deuterium, or halogen. In some embodiments, R¹ is hydrogen, or C₁₋₃alkyl optionally substituted with deuterium.

In some embodiments, R¹ is hydrogen, methyl, methyl-d3, ethyl, isopropyl, 2-hydroxyethyl, 2-methoxyethyl, 2,2,2-trifluoroethyl, 2,2-difluoroethyl, or cyclopropylmethyl. In some embodiments, R¹ is hydrogen, methyl, ethyl or methyl-d3. In some embodiments, R¹ is methyl or methyl-d3. In some embodiments, R¹ is methyl.

The definitions of R²

In some embodiments, R² is hydrogen, halogen, alkyl, alkoxyl, or cyano, provided that R² is absent when X¹ is N and the bond [symbol] attached to X¹ is a double bond. In some embodiments, R² is hydrogen, halogen, C₁₋₄ alkyl, C₁₋₄ alkoxyl or cyano. In some embodiments, R² is hydrogen, F, Br, Cl or CN. In some embodiments, R² is hydrogen.

In some embodiments, R² is hydrogen, halogen, C₁₋₄alkyl, C₁₋₄ alkoxyl or cyano; preferably R² is hydrogen, F, Br, Cl or CN; more preferably R² is hydrogen, F, or CN; even more preferably R² is hydrogen.

The definitions of R⁴

In some embodiments, R⁴ is hydrogen, halogen or alkyl, wherein the alkyl is optionally substituted with halogen or —OR^{4a}, wherein R^{4a} is hydrogen, alkyl, alkenyl, alkynyl, cycloalkyl or heterocycloalkyl, wherein alkyl, alkenyl, alkynyl, cycloalkyl or heterocycloalkyl is optionally substituted with —C₁₋₆aklyl, —C₁₋₆alkoxy or —C₃₋₈cycloalkyl. In some embodiments, R⁴ is hydrogen, halogen or C₁₋₄alkyl, wherein the alkyl is optionally substituted with halogen or —OR⁴a. In some embodiments, R⁴ is hydrogen, halogen or C₁₋₄alkyl, wherein the alkyl is optionally substituted with halogen.

In some embodiments, R⁴ is hydrogen, fluoro, chloro, bromo, methyl, trifluoromethyl, ethyl, or 2,2,2-trifluoroethyl. In some embodiments, R⁴ is hydrogen.

The definitions of R⁵

In some embodiments, R⁵ is hydrogen, alkyl, alkenyl, alkynyl, cycloalkyl, R^{5a}—C(O)—, R^{5a}—C(O)O—, R^{5a}—O—C(O)—, R^{5a}—C(O)NR^{5b}—, R^{5a}—NR^{5b}—C(O)—, R^{5a}—SO₂— or heterocyclyl, wherein said alkyl is unsubstituted or substituted with cyano, —C(O)OR^{5c}, —C(O)R^{5c}, —C(O)NR^{5c}R^{5d}, heterocyclyl, alkoxy, hydroxy, cycloalkyl, or NR^{5c}R_{5d}; and wherein each of said cycloalkyl and heterocyclyl is unsubstituted or substituted with alkyl, cyano or halogen substituted alkyl, cyano, —C(O)OR^{5c}, —C(O)R^{5c}, —C(O)NR^{5c}R^{5d}, heterocyclyl, alkoxy, hydroxy, cycloalkyl, NR^{5c}R^{5d}, or R^{5c}—SO₂—, wherein R^{5a} and R^{5b} are each independently hydrogen, alkyl, or cycloalkyl; and wherein R^{5c} and R^{5d} are hydrogen or alkyl.

In some embodiments, R⁵ is hydrogen, alkyl, alkenyl or alkynyl, wherein said alkyl is unsubstituted or substituted with cyano. In some embodiments, R⁵ is C₁₋₄alkyl, C₂₋₄alkenyl or C₂₋₄alkynyl, wherein said alkyl is substituted with cyano. In some embodiments, R⁵ is C₁₋₄alkyl, wherein said alkyl is substituted with cyano.

In some embodiments, R⁵ is C₁₋₄alkyl, wherein said alkyl is substituted with cyano, alkoxy, hydroxy, NR^{5c}R^{5d}, ═NO—CH₃, —S(═O)—CH₃, —S(═O)₂—CH₃, or —S(═O)(═NH)—CH₃.

In some embodiments, R⁵ is hydrogen, —CH₂—CN, —CH₂C(O)—OMe, —CH(CH₃)CN, oxiran-2-ylmethyl-, prop-2-yn-1-yl, but-2-yn-1-yl, prop-1-en-2-yl, methyl, isopropyl, —CH₂CH₂—O—CH₃, —CH₂—O—CH₃, —CH(CH₃)—O—CH₃, —CH₂C(O)NH₂, —CH₂CH₂—OH, —CH₂—OH, cyclopropyl-CH₂—, —CH₂CH₂N(CH₃)₂, CH₃—SO₂—, cyclopropyl, cyclobutyl, cyclopropyl-C(O)—, 1-cyanocyclopropyl, 2-cyanocyclopropyl, 2-cyanocyclobutyl, 3-(cyanomethyl)-1-(ethylsulfonyl)azetidine-3-yl, 1-cyano-2-cyclopentyleth-2-yl, —CH₂—N(CH₃)₂, —CH═N—O—CH₃, —CH₂—S(═O)—CH₃, —CH₂—S(═O)₂—CH₃, or —CH₂—S(═O)(═NH)—CH₃; preferably, R⁵ is hydrogen, CN—CH₂—, —CH₂C(O)—OMe, —CH(CH₃)CN, oxiran-2-ylmethyl-, prop-2-yn-1-yl, but-2-yn-1-yl, prop-1-en-2-yl, —CH₂—OH, —CH(CH₃)CN, —CH₂—O—CH₃, —CH(CH₃)—O—CH₃, —CH₂—N(CH₃)₂, —CH═NO—CH₃, —CH₂—S(═O)—CH₃, —CH₂—S(═O)₂—CH₃, or —CH₂—S(═O)(═NH)—CH₃; more preferably, R⁵ is CN—CH₂—, prop-2-yn-1-yl, but-2-yn-1-yl, or prop-1-en-2-yl, —CH₂—OH, —CH(CH₃)CN, —CH₂—O—CH₃, —CH(CH₃)—O—CH₃, —CH₂—N(CH₃)₂, —CH═NO—CH₃, —CH₂—S(═O)—CH₃, —CH₂—S(═O)₂—CH₃, or —CH₂—S(═O)(═NH)—CH₃; more preferably R⁵ is CN—CH₂—, —CH₂—OH, —CH(CH₃)CN, —CH₂—O—CH₃, —CH(CH₃)—O—CH₃, —CH₂—N(CH₃)₂, —CH═NO—CH₃, —CH₂—S(═O)—CH₃, —CH₂—S(═O)₂—CH₃, or —CH₂—S(═O)(═NH)—CH₃; more preferably R⁵ is CN—CH₂—.

The definitions of R⁶

In some embodiments, R⁶ is absent.

In some embodiments, R⁶ is absent, hydrogen, halogen, alkyl which is unsubstituted or substituted with halogen or cyano, provided that R⁶ is absent when the bond [symbol] attached to the nitrogen to which R⁶ is attached is a double bond. In some embodiments, R⁶ is hydrogen, F, Br, Cl or C₁₋₄alkyl which is unsubstituted or substituted with cyano.

The definitions of R⁷/R⁹, R⁸/R¹⁰

In some embodiments, each of R⁷, R⁹, R⁸, and R¹⁰ is independently hydrogen, alkyl, or —C(O)R^{7a}, wherein said alkyl is unsubstituted or substituted with halogen, cyano, hydroxy, or alkoxy, and wherein R^{7a} is hydrogen, alkyl, or alkoxy, provided that at least one of R⁷ and R⁹ is not hydrogen.

In some embodiments, each of R⁷ and R⁹ is independently hydrogen, alkyl, or —C(O)R^{7a}, wherein said alkyl is unsubstituted or substituted with halogen, cyano, hydroxy, or alkoxy, and wherein R^{7a} is hydrogen, alkyl, or alkoxy. In some embodiments, each of R⁷ and R⁹ is independently C₁₋₄alkyl. In some embodiments, each of R⁷ and R⁹ is independently C₁₋₂alkyl.

In some embodiments, R⁷ and R⁹ are each independently hydrogen, methyl, ethyl, methoxymethyl, 1-hydroxyethyl, 2-methoxyethyl, cyanomethyl, hydroxyethyl, hydroxymethyl, methoxycarbonyl, difluoromethyl, provided that at least one of R⁷ and R⁹ is not hydrogen.

In some embodiments, R⁸ and R¹⁰ are each hydrogen.

In some embodiments, R⁷ is methyl, and R⁹ is methyl; or R⁷ is ethyl, and R⁹ is ethyl; or R⁷ is methyl, and R⁹ is ethyl; or R⁷ is methyl, and R⁹ is methoxycarbonyl; or R⁷ is hydrogen, and R⁹ is methyl; or R⁷ is hydrogen, and R⁹ is ethyl.

In some embodiments, R⁷ and R⁹ are each hydrogen and R⁸ and R¹⁰ together form a bridge containing at least one —CH₂— moiety in addition to the two bridgehead atoms. In some embodiments, R⁷ and R⁹ are each hydrogen and R⁸ and R¹⁰ together form a bridge containing one —CH₂— moiety in addition to the two bridgehead atoms. In some embodiments, R⁸ and R⁹ are each hydrogen and R⁸ and R¹⁰ together form a bridge containing two —CH₂— moieties in addition to the two bridgehead atoms.

In some embodiments, R⁸ and R¹⁰ are each hydrogen and R⁷ and R⁹ together form a bridge containing at least one —CH₂— moiety in addition to the two bridgehead atoms. In some embodiments, R⁸ and R¹⁰ are each hydrogen and R⁷ and R⁹ together form a bridge containing one —CH₂— moiety in addition to the two bridgehead atoms. In some embodiments, R⁸ and R¹⁰ are each hydrogen and R⁸ and R⁹ together form a bridge containing two —CH₂— moieties in addition to the two bridgehead atoms.

In some embodiments, R⁸ and R¹⁰ are each hydrogen.

In some embodiments, R⁸ and R¹⁰ are each hydrogen; R⁷ is methyl; and R⁹ is methyl. In some embodiments, R⁸ and R¹⁰ are each hydrogen; R⁷ is ethyl; and R⁹ is ethyl. In some embodiments, R⁸ and R¹⁰ are each hydrogen; R⁷ is methyl; and R⁹ is ethyl. In some embodiments, R⁸ and R¹⁰ are each hydrogen; R⁷ is ethyl, and R⁹ is methyl.

The definitions of L¹

In some embodiments, L¹ is a direct bond, —O—, —N(R^{L})—, -alkylene- or —C(O)—, wherein R^{L} is hydrogen or alkyl and wherein said -alkylene- is unsubstituted or substituted with halogen, alkoxy, or heterocyclyl. In some embodiments, L¹ is a direct bond, —O—, —N(R^{L})—, -alkylene- or —C(O)—, wherein R^{L} is hydrogen or alkyl. In some embodiments, L¹ is C₁₋₄alkylene, preferably C₁₋₂alkylene. In some embodiments, L¹ is a direct bond, —CH₂—, —CH(CH₃)—, —CH(CH₂CH₃)—, —CH(CHF₂)—, —N(H)—, —N(CH₃)—, —O—, —CH(C(O)—NHCH₂CH₂OCH₃)— or —C(CH₃)₂—. In some embodiments, L¹ is —CH₂— or —CH(CH₃)—.

The definition of X² and X³

In some embodiments, X² and X³ are independently N or CH. In some embodiments, X² is N, and X³ is N. In some embodiments, X² is N, and X³ is CH. In some embodiments, X² is CH, and X³ is N. In some embodiments, X² is CH, and X³ is CH.

The definitions of Cy¹

In some embodiments, Cy¹ is aryl, heterocyclyl, heterocyclyl, heteroaryl, or cycloalkyl, each of which is unsubstituted or substituted with one, two or three substituents R^{3a}, wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, or heterocyclyl, wherein said alkyl moiety in the group alkyl or alkoxy is unsubstituted or substituted with halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl.

In some embodiments, Cy¹ is aryl, which is unsubstituted or substituted with one, two or three substituents R^{3a}, wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, or heterocyclyl, wherein said alkyl moiety in the group alkyl or alkoxy is unsubstituted or substituted with halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl.

In some embodiments, Cy¹ is aryl, which is unsubstituted or substituted with one, two or three substituents R^{3a}, wherein R^{3a} is selected from alkoxy, halogen-substituted alkoxy, alkoxyalkyl-, alkyl, halogen-substituted alkyl, halogen, R^{3b}—SO₂—, cycloalkyl, hydroxyalkyl-, cyano, R^{3b}—C(O)—N(R^{3c})—, cyano-substituted alkyl, N(R^{3b}R^{3c})—C(O)—, R^{3b}—O—C(O)—, or heterocyclyl, said cycloalkyl or heterocyclyl is unsubstituted or substituted with alkoxy, alkyl, halogen, or hydroxy, wherein R^{3b} and R^{3c} are each independently hydrogen or alkyl.

In some embodiments, Cy¹ is unsubstituted or substituted cycloalkyl, unsubstituted or substituted aryl, unsubstituted or substituted heterocyclyl, or unsubstituted or substituted heteroaryl. In some embodiments, Cy¹ is optionally substituted with one, two or three substituents R^{3a}, wherein R^{3a} is selected from deuterium, fluoro, bromo, chloro, methyl, difluoromethyl, trifluoromethoxy, methoxy, methoxymethyl, trifluoromethyl, methylsulfonyl, difluoro, ethoxy, isopropyl, isopropoxy, difluoromethoxy, cyclopropyl, 2,2-difluorocyclopropyl, 1-fluorocyclopropyl, 2-fluorocyclopropyl, cyclobutyl, 1-hydroxyethyl, 2-hydroxyethyl, ethyl, 1,1-difluoroethyl, cyano, dimethoxy, dichloro, acetamido, 1-methoxyethyl, cyanomethyl, carbamoyl, methoxycarbonyl, dimethylcarbamoyl, (difluoromethoxy)methyl, amino, 1-(difluoromethoxy)ethyl, azetidin-1-yl, 2-methoxypropan-2-yl, 1-methoxycyclopropyl, oxetan-3-yl, 1-methylazetidin-3-yl, 1-hydroxyazetidin-3-yl, 2-cyanoethyl, 2-cyanopropan-2-yl, hydroxymethyl, 3-hydroxypropyl, 2-hydroxypropan-2-yl, or 2-fluoropropan-2-yl.

In some embodiments, Cy¹ is an aryl of from 6 to 14 carbon atoms having a single ring or multiple condensed rings which is unsubstituted or substituted with one, two or three R^{3a}. In some embodiments, said aromatic carbocyclic group is phenyl, naphthyl or anthryl, indanyl, or tetrahydronaphthyl, which is unsubstituted or substituted with one, two or three R^{3a}.

In some embodiments, Cy¹ is phenyl. In some embodiments, Cy¹ is phenyl, which is substituted with one R^{3a} as disclosed herein at position 4 and optionally substituted with R^{3a} on the other position.

In some embodiments, Cy¹ is naphthalenyl. In some embodiments, Cy¹ is naphthalen-1-yl, naphthalen-2-yl, naphthalen-3-yl, naphthalen-4-yl.

In some embodiments, Cy¹ is benzo[d][1,3]dioxol-5-yl, 2,2-dimethylbenzo[d][1,3]dioxol-5-yl, 2,2-dimethylbenzo[d][1,3]dioxol-5-yl, 2,3-dihydrobenzo[b][1,4]dioxin-6-yl, 3,3-dimethyl-2,3-dihydrobenzo[b][1,4]dioxin-6-yl, 3,3-difluoro-2,3-dihydrobenzo[b][1,4]dioxin-6-yl, 2,2-difluorobenzo[d][1,3]dioxol-4-yl, 2,2-dimethylbenzo[d][1,3]dioxol-4-yl, 3,4-dihydro-2H-benzo[b][1,4]oxazin-6-yl, or

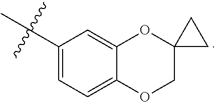 [SMILES: *c1ccc2c(c1)OC1(CC1)CO2]

In some embodiments, Cy¹ is a monocyclic 5- to 9-membered heterocyclyl or a bicyclic 7- to 10-membered heterocyclyl which is unsubstituted or substituted with one, two or three R^{3a}, wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, or heterocyclyl, wherein said alkyl moiety in the group alkyl or alkoxy is unsubstituted or substituted with halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl; preferably, R^{3a} is selected from alkoxy, halogen-substituted alkoxy, alkoxyalkyl-, alkyl, halogen-substituted alkyl, halogen, R^{3b}—SO₂—, cycloalkyl, hydroxyalkyl-, cyano, R^{3b}—C(O)—N(R^{3c})—, cyano-substituted alkyl, N(R^{3b}R^{3c})—C(O)—, R^{3b}—O—C(O)—, or heterocyclyl, said cycloalkyl or heterocyclyl is unsubstituted or substituted with alkoxy, alkyl, halogen, or hydroxy, wherein R^{3b} and R^{3c} are each independently hydrogen or alkyl. In some embodiments, said monocyclic 5- to 9-membered heterocyclyl is tetrahydrofuranyl, tetrahydropyranyl, 1,4-dioxanyl, piperidinyl, piperazinyl, or dihydropyridinyl, each of which is unsubstituted or substituted with one, two or three R^{3a} as disclosed herein. In some embodiments, Cy¹ is tetrahydrofuran-2-yl, tetrahydrofuran-3-yl, tetrahydro-2H-pyran-2-yl, tetrahydro-2H-pyran-3-yl, tetrahydro-2H-pyran-4-yl, 1,4-dioxan-2-yl, 1,4-dioxan-3-yl, piperidin-1-yl, piperidin-2-yl, piperidin-3-yl, piperidin-4-yl, piperazin-1-yl, piperazin-2-yl, piperazin-3-yl, piperazin-4-yl, 1,2-dihydropyridin-3-yl, 1,2-dihydropyridin-4-yl, 1,2-dihydropyridin-5-yl, or 1,2-dihydropyridin-6-yl.

In some embodiments, Cy¹ is piperidinyl (e.g., piperidin-1-yl) or piperazinyl (e.g., piperazin-4-yl), which is substituted with one R^{3a} as disclosed herein at position 4 and optionally substituted with R^{3a} on the other position.

In some embodiments, said bicyclic 7- to 10-membered heterocyclyl is chromanyl, preferably chroman-2-yl, chroman-3-yl, or chroman-4-yl.

In some embodiments, Cy¹ is a monocyclic 5- to 9-membered heteroaryl or a bicyclic 7- to 10-membered heteroaryl which is unsubstituted or substituted with one, two or three R^{3a}, wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, or heterocyclyl, wherein said alkyl moiety in the group alkyl or alkoxy is unsubstituted or substituted with halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl; preferably, R^{3a} is selected from deuterium, alkoxy, halogen-substituted alkoxy, alkoxyalkyl-, alkyl, halogen-substituted alkyl, halogen, R^{3b}—SO₂—, cycloalkyl, hydroxyalkyl-, cyano, R^{3b}—C(O)—N(R^{3c})—, cyano-substituted alkyl, N(R^{3b}R^{3c})—C(O)—, N(R^{3b}R^{3c})—, R^{3b}—O—C(O)—, or heterocyclyl, said cycloalkyl or heterocyclyl is unsubstituted or substituted with alkoxy, alkyl, halogen, or hydroxy, wherein R^{3b} and R^{3c} are each independently hydrogen or alkyl; more preferably R^{3a} is selected from deuterium, fluoro, bromo, chloro, methyl, difluoromethyl, trifluoromethoxy, methoxy, methoxymethyl, trifluoromethyl, methylsulfonyl, difluoro, ethoxy, isopropoxy, trifluoromethoxy, difluoromethoxy, cyclopropyl, 1-hydroxyethyl, ethyl, 1,1-difluoroethyl, cyano, dimethoxy, dichloro, cyclopropyl-, acetamido, 1-methoxyethyl, cyanomethyl, carbamoyl, methoxycarbonyl, dimethylcarbamoyl, (difluoromethoxy)methyl, amino, 1-(difluoromethoxy)ethyl, azetidin-1-yl, 2-methoxypropan-2-yl, 1-methoxycyclopropyl, oxetan-3-yl, 1-methylazetidin-3-yl, or 1-hydroxyazetidin-3-yl.

In some embodiments, said monocyclic 5- to 9-membered heteroaryl is pyrazolyl, imidazolyl, pyridinyl, pyrimidinyl, pyrazinyl or pyridazinyl, each of which is unsubstituted or substituted with one, two or three R^{3a} as disclosed herein. In some embodiments, said monocyclic 5- to 9-membered heteroaryl is 1H-imidazol-2-yl, 1H-imidazol-4-yl, 1H-imidazol-5-yl, pyridin-2-yl, pyridin-3-yl, pyridin-4-yl, pyrimidin-2-yl, pyrimidin-4-yl, pyrimidin-5-yl, 1H-indol-2-yl, 1H-indol-3-yl, 1H-indol-4-yl, 1H-indol-5-yl, 1H-indol-6-yl, 1H-indol-7-yl, pyrazin-2-yl, or pyridazin-4-yl, each of which is unsubstituted or substituted with one, two or three R^{3a} as disclosed herein.

In some embodiments, said bicyclic 7- to 10-membered heteroaryl is indolyl, benzo[d]imidazolyl, triazolopyridinyl, imidazopyridinyl, benzooxazolyl, benzo[d]thiazolyl, quinolinyl, isoquinolinyl, naphthyridinyl, dioxinopyridineyl, quinoxalinyl, benzo[d]imidazolyl, imidazo[4,5-b]pyridinyl, thiazolo[5,4-b]pyridinyl, thiazolo[4,5-b]pyridinyl, thieno[2,3-b]pyridinyl, or thieno[3,2-b]pyridinyl, each of which is unsubstituted or substituted with one, two or three R^{3a} as disclosed herein. In some embodiments, said bicyclic 7- to 10-membered heteroaryl is 1H-benzo[d]imidazol-2-yl, 1H-benzo[d]imidazol-4-yl, 1H-benzo[d]imidazol-5-yl, 1H-benzo[d]imidazol-6-yl, 1H-benzo[d]imidazol-7-yl, [1,2,4]triazolo[1,5-a]pyridin-2-yl, [1,2,4]triazolo[1,5-a]pyridin-5-yl, [1,2,4]triazolo[1,5-a]pyridin-6-yl, [1,2,4]triazolo[1,5-a]pyridin-7-yl, [1,2,4]triazolo[1,5-a]pyridin-8-yl, 3H-imidazo[4,5-b]pyridine-2-yl, 3H-imidazo[4,5-b]pyridine-5-yl, 3H-imidazo[4,5-b]pyridine-6-yl, 3H-imidazo[4,5-b]pyridine-7-yl, 1H-imidazo[4,5-b]pyridin-2-yl, 1H-imidazo[4,5-b]pyridin-5-yl, 1H-imidazo[4,5-b]pyridin-6-yl, 1H-imidazo[4,5-b]pyridin-7-yl, benzo[d]oxazol-2-yl, benzo[d]oxazol-4-yl, benzo[d]oxazol-5-yl, benzo[d]oxazol-6-yl, benzo[d]oxazol-7-yl, benzo[d]thiazol-2-yl, benzo[d]thiazol-4-yl, benzo[d]thiazol-5-yl, benzo[d]thiazol-6-yl, benzo[d]thiazol-7-yl, quinolin-2-yl, quinolin-3-yl, quinolin-4-yl, quinolin-5-yl, quinolin-6-yl, quinolin-7-yl, isoquinolin-1-yl, isoquinolin-3-yl, isoquinolin-4-yl, isoquinolin-5-yl, isoquinolin-6-yl, isoquinolin-7-yl, isoquinolin-8-yl, quinoxalin-2-yl, quinoxalin-3-yl, quinoxalin-4-yl, quinoxalin-5-yl, quinoxalin-6-yl, quinoxalin-7-yl, quinoxalin-8-yl, 1,8-naphthyridin-2-yl, 1,8-naphthyridin-3-yl, 1,8-naphthyridin-4-yl, 2,3-dihydro-[1,4]dioxino[2,3-b]pyridine-6-yl, 2,3-dihydro-[1,4]dioxino[2,3-b]pyridine-7-yl, 2,3-dihydro-[1,4]dioxino[2,3-b]pyridine-8-yl, quinoxalin-6-yl-2,3-d2, 1H-indol-2-yl, 1H-benzo[d]imidazol-2-yl, 1-methyl-1H-benzo[d]imidazol-6-yl, 3H-imidazo[4,5-b]pyridin-2-yl, 4,5,6,7-tetrahydro-1H-benzo[d]imidazol-2-yl, 2,3-dihydro-[1,4]dioxino[2,3-b]pyridin-6-yl, thiazolo[5,4-b]pyridin-2-yl, thiazolo[5,4-b]pyridin-5-yl, thiazolo[5,4-b]pyridin-6-yl, thiazolo[5,4-b]pyridin-7-yl, thiazolo[4,5-b]pyridin-2-yl, thiazolo[4,5-b]pyridin-5-yl, thiazolo[4,5-b]pyridin-6-yl, thiazolo[4,5-b]pyridin-7-yl, thieno[2,3-b]pyridin-2-yl, thieno[2,3-b]pyridin-3-yl, thieno[2,3-b]pyridin-4-yl, thieno[2,3-b]pyridin-5-yl, thieno[2,3-b]pyridin-6-yl, thieno[3,2-b]pyridin-2-yl, thieno[3,2-b]pyridin-3-yl, thieno[3,2-b]pyridin-5-yl, thieno[3,2-b]pyridin-6-yl, thieno[3,2-b]pyridin-7-yl, each of which is unsubstituted or substituted with one, two or three R^{3a} as disclosed herein.

In some embodiments, Cy¹ is quinoxalinyl, e.g., quinoxalin-2-yl, quinoxalin-3-yl, quinoxalin-4-yl, quinoxalin-5-yl, quinoxalin-6-yl, quinoxalin-7-yl, quinoxalin-8-yl, preferably quinoxalin-6-yl, which is unsubstituted or substituted with one, two or three R^{3a}, wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, or heterocyclyl, wherein said alkyl moiety in the group alkyl or alkoxy is unsubstituted or substituted with halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl; preferably, R^{3a} is selected from deuterium, alkoxy, halogen-substituted alkoxy, alkoxyalkyl-, alkyl, halogen-substituted alkyl, halogen, R^{3b}—SO₂—, cycloalkyl, hydroxyalkyl-, cyano, R^{3b}—C(O)—N(R^{3c})—, cyano-substituted alkyl, N(R^{3b}R^{3c})—C(O)—, N(R^{3b}R^{3c})—, R^{3b}—O—C(O)—, or heterocyclyl, said cycloalkyl or heterocyclyl is unsubstituted or substituted with alkoxy, alkyl, halogen, or hydroxy, wherein R^{3b} and R^{3c} are each independently hydrogen or alkyl; more preferably R^{3a} is selected from deuterium, fluoro, bromo, chloro, methyl, difluoromethyl, trifluoromethoxy, methoxy, methoxymethyl, trifluoromethyl, methylsulfonyl, difluoro, ethoxy, isopropoxy, trifluoromethoxy, difluoromethoxy, cyclopropyl, 1-hydroxyethyl, ethyl, 1,1-difluoroethyl, cyano, dimethoxy, dichloro, cyclopropyl-, acetamido, 1-methoxyethyl, cyanomethyl, carbamoyl, methoxycarbonyl, dimethylcarbamoyl, (difluoromethoxy)methyl, amino, 1-(difluoromethoxy)ethyl, azetidin-1-yl, 2-methoxypropan-2-yl, 1-methoxycyclopropyl, oxetan-3-yl, 1-methylazetidin-3-yl, or 1-hydroxyazetidin-3-yl.

In some embodiments, Cy¹ is quinoxalin-6-yl, which is unsubstituted or substituted with one, two or three R^{3a}, wherein R^{3a} is deuterium, methyl, ethyl, isopropyl, cyanomethyl, 2-cyanoethyl, 2-cyanopropan-2-yl, hydroxymethyl, 2-hydroxyethyl, 1-hydroxyethyl, 3-hydroxypropyl, 2-hydroxypropan-2-yl, difluoromethyl, trifluoromethyl, 2-fluoropropan-2-yl, methoxy, isopropoxy, difluoromethoxy, fluoro, chloro, cyano, amino, cyclopropyl, 2,2-difluorocyclopropyl, 1-fluorocyclopropyl, 2-fluorocyclopropyl, or cyclobutyl; preferably deuterium, methyl, ethyl, trifluoromethyl, methoxy, isopropoxy, difluoromethoxy, fluoro, chloro, cyano, amino, or cyclopropyl. In some embodiments, Cy¹ is quinoxalin-6-yl, or 3-methyl-quinoxalin-6-yl.

In some embodiments, Cy¹ is benzo[d]thiazol-5-yl or benzo[d]thiazol-6-yl, each of which is unsubstituted or substituted with one, two or three R^{3a}, wherein R^{3a} is deuterium, methyl, ethyl, isopropyl, cyanomethyl, 2-cyanoethyl, 2-cyanopropan-2-yl, hydroxymethyl, 2-hydroxyethyl, 1-hydroxyethyl, 3-hydroxypropyl, 2-hydroxypropan-2-yl, difluoromethyl, trifluoromethyl, 2-fluoropropan-2-yl, methoxy, isopropoxy, difluoromethoxy, fluoro, chloro, cyano, amino, cyclopropyl, 2,2-difluorocyclopropyl, 1-fluorocyclopropyl, 2-fluorocyclopropyl, or cyclobutyl; preferably deuterium, methyl, ethyl, trifluoromethyl, methoxy, isopropoxy, difluoromethoxy, fluoro, chloro, cyano, amino, or cyclopropyl.

In some embodiments, Cy¹ is thiazolo[5,4-b]pyridin-5-yl, thiazolo[5,4-b]pyridin-6-yl, thiazolo[5,4-b]pyridin-7-yl, thiazolo[4,5-b]pyridin-5-yl, thiazolo[4,5-b]pyridin-6-yl, thiazolo[4,5-b]pyridin-7-yl, each of which is unsubstituted or substituted with one, two or three R^{3a}, wherein R^{3a} is deuterium, methyl, ethyl, isopropyl, cyanomethyl, 2-cyanoethyl, 2-cyanopropan-2-yl, hydroxymethyl, 2-hydroxyethyl, 1-hydroxyethyl, 3-hydroxypropyl, 2-hydroxypropan-2-yl, difluoromethyl, trifluoromethyl, 2-fluoropropan-2-yl, methoxy, isopropoxy, difluoromethoxy, fluoro, chloro, cyano, amino, cyclopropyl, 2,2-difluorocyclopropyl, 1-fluorocyclopropyl, 2-fluorocyclopropyl, or cyclobutyl; preferably deuterium, methyl, ethyl, trifluoromethyl, methoxy, isopropoxy, difluoromethoxy, fluoro, chloro, cyano, amino, or cyclopropyl.

In some embodiments, Cy¹ is thieno[2,3-b]pyridin-6-yl or thieno[3,2-b]pyridin-5-yl, each of which is unsubstituted or substituted with one, two or three R^{3a}, wherein R^{3a} is deuterium, methyl, ethyl, isopropyl, cyanomethyl, 2-cyanoethyl, 2-cyanopropan-2-yl, hydroxymethyl, 2-hydroxyethyl, 1-hydroxyethyl, 3-hydroxypropyl, 2-hydroxypropan-2-yl, difluoromethyl, trifluoromethyl, 2-fluoropropan-2-yl, methoxy, isopropoxy, difluoromethoxy, fluoro, chloro, cyano, amino, cyclopropyl, 2,2-difluorocyclopropyl, 1-fluorocyclopropyl, 2-fluorocyclopropyl, or cyclobutyl; preferably deuterium, methyl, ethyl, trifluoromethyl, methoxy, isopropoxy, difluoromethoxy, fluoro, chloro, cyano, amino, or cyclopropyl.

In some embodiments, Cy¹ is
phenyl, 2-(trifluoromethoxy)phenyl, 2-methoxyphenyl, 2-(methoxymethyl)phenyl, 2-(trifluoromethyl)phenyl, 4-fluoro-2-(methoxymethyl)phenyl, 4-fluoro-2-methoxyphenyl, 4-fluoro-2-methylphenyl, 2-bromo-4-fluorophenyl, 4-fluoro-2-(methylsulfonyl)phenyl, 4-methyl-2-(trifluoromethyl)phenyl, 2-chloro-4-fluorophenyl, 2,4-difluorophenyl, 2-ethoxy-4-fluorophenyl, 4-fluoro-2-isopropoxyphenyl, 4-fluoro-2-(trifluoromethoxy)phenyl, 2-(difluoromethoxy)-4-fluorophenyl, 2-(difluoromethyl)-4-fluorophenyl, 2-cyclopropyl-4-fluorophenyl, 4-fluoro-2-(1-hydroxyethyl)phenyl, 4-cyclopropyl-2-methoxyphenyl,2-ethyl-4-fluorophenyl, 4-fluoro-2-(trifluoromethyl)phenyl, 2-methoxy-4-fluorophenyl, 2-(1,1-difluoroethyl)-4-fluorophenyl, 2-cyano-4-fluorophenyl, 4-fluoro-3-(methoxymethyl)phenyl, 3-methyl-2-(trifluoromethyl)phenyl, 4-fluoro-2,6-dimethoxyphenyl, 2,4-difluoro-6-methoxyphenyl, 2,6-dichloro-4-fluorophenyl, 4-cyclopropylphenyl, 4-methoxyphenyl, 4-fluorophenyl, 4-cyclopropyl-phenyl, 4-(trifluoromethyl)phenyl, 4-methylphenyl, 4-(difluoromethyl)phenyl, 4-isopropoxyphenyl, 2-fluoro-4-(trifluoromethyl)phenyl, 4-cyclopropyl-2-fluorophenyl, 3-methoxy-4-(trifluoromethyl)phenyl, 4-fluoro-3-methoxyphenyl, 2,6-difluorophenyl, 4-(trifluoromethoxy)phenyl, 4-acetamidophenyl, 4-fluoro-2-(1-methoxyethyl)phenyl, 2-(cyanomethyl)-4-fluorophenyl, 3,4-difluoro-2-(trifluoromethyl)phenyl, 2-carbamoyl-4-fluorophenyl, 2-methoxycarbonyl-4-fluorophenyl, 2-(dimethylcarbamoyl)-4-fluorophenyl, 2-((difluoromethoxy)methyl)-4-fluorophenyl, 2-(1-(difluoromethoxy)ethyl)-4-fluorophenyl, 2-(azetidin-1-yl)-4-fluorophenyl, 4-fluoro-2-(2-methoxypropan-2-yl)phenyl, 3-(trifluoromethyl)phenyl, 4-fluoro-2-(1-methoxycyclopropyl)phenyl, 4-fluoro-2-(oxetan-2-yl)phenyl, 4-fluoro-2-(1-methylazetidin-3-yl)phenyl, 4-fluoro-2-(1-hydroxyazetidin-3-yl)phenyl, 4-cyclopropyl-2-methoxyphenyl; 2-ethyl-4-fluorophenyl; 2,6-difluoro-4-methoxyphenyl; 2,5-difluoro-4-methoxyphenyl; or naphthalen-2-yl; or
benzo[d][1,3]dioxol-5-yl, 2,2-dimethylbenzo[d][1,3]dioxol-5-yl, 2,2-dimethylbenzo[d][1,3]dioxol-5-yl, 2,3-dihydrobenzo[b][1,4]dioxin-6-yl, 3,3-dimethyl-2,3-dihydrobenzo[b][1,4]dioxin-6-yl, 3,3-difluoro-2,3-dihydrobenzo[b][1,4]dioxin-6-yl, 2,2-difluorobenzo[d][1,3]dioxol-4-yl, 2,2-dimethylbenzo[d][1,3]dioxol-4-yl, 3,4-dihydro-2H-benzo[b][1,4]oxazin-6-yl, or

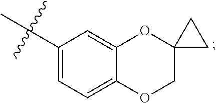 [SMILES: *c1ccc2c(c1)OC1(CC1)CO2]

or
tetrahydrofuran-2-yl, tetrahydrofuran-3-yl, tetrahydro-2H-pyran-2-yl, tetrahydro-2H-pyran-3-yl, tetrahydro-2H-pyran-4-yl, 1,4-dioxan-2-yl, 1,4-dioxan-3-yl, piperidin-2-yl, piperidin-3-yl, piperidin-4-yl, piperazin-2-yl, piperazin-3-yl, 1,2-dihydropyridin-3-yl, 1,2-dihydropyridin-4-yl, 1,2-dihydropyridin-5-yl, or 1,2-dihydropyridin-6-yl; or
chroman-2-yl, chroman-3-yl, or chroman-4-yl; or
1H-pyrazol-3-yl, 1H-pyrazol-4-yl, 1H-pyrazol-5-yl, 1-ethyl-5-(trifluoromethyl)-1H-pyrazol-4-yl; 5-ethyl-1-methyl-1H-pyrazol-4-yl; 1H-imidazol-2-yl, 1H-imidazol-4-yl, 1H-imidazol-5-yl, pyridin-2-yl, pyridin-3-yl, pyridin-4-yl, pyrimidin-2-yl, pyrimidin-4-yl, pyrimidin-5-yl, 1H-indol-2-yl, 1H-indol-3-yl, 1H-indol-4-yl, 1H-indol-5-yl, 1H-indol-6-yl, 1H-indol-7-yl, 3-methoxypyridin-2-yl, 6-isopropoxypyridin-3-yl, 2-methoxypyridin-3-yl, 2-(trifluoromethyl)pyridin-3-yl, 6-(difluoromethoxy)pyridin-3-yl, 3-methoxypyridin-4-yl, 3-(trifluoromethyl)pyridin-4-yl, 5-fluoro-3-(trifluoromethyl)pyridin-2-yl, 5-chloro-1-ethyl-1H-imidazol-2-yl, 1-ethyl-2-(trifluoromethyl)-1H-imidazol-5-yl, 1-ethyl-5-(trifluoromethyl)-1H-pyrazol-4-yl, 5-ethyl-1-methyl-1H-pyrazol-4-yl, 1-methyl-5-(trifluoromethyl)-1H-pyrazol-4-yl, 5-(difluoromethyl)-1-methyl-1H-pyrazol-4-yl, 1-ethyl-3-(trifluoromethyl)-1H-pyrazol-5-yl, 3-chloro-1-ethyl-1H-pyrazol-5-yl, 1-ethyl-4-methyl-1H-pyrazol-5-yl, 5-isopropoxypyridin-2-yl, 6-(trifluoromethyl)pyridin-3-yl, 3,5-difluoropyridin-2-yl, 3,5-difluoropyridin-4-yl, 1-ethyl-4-cyano-1H-pyrazol-3-yl, 5-fluoropyridin-2-yl, pyrazin-2-yl, 3-(trifluoromethyl)pyridazin-4-yl, 6-cyclopropylpyridin-3-yl, 6-cyclobutylpyridin-3-yl, 6-isopropylpyridin-3-yl, 6-cyanopyridin-3-yl, 6-(2-cyanopropan-2-yl)pyridin-3-yl, 6-(2-hydroxypropan-2-yl)pyridin-3-yl, 6-cyclopropyl-2-fluoropyridin-3-yl, 6-methoxypyridin-3-yl, 6-(2-fluoropropan-2-yl)pyridin-3-yl, 5-methoxypyridin-2-yl, 5-cyclopropylpyridin-2-yl, 6-(2,2-difluorocyclopropyl)pyridin-3-yl, 5-(difluoromethyl)pyridin-2-yl, 5-(trifluoromethyl)pyridin-2-yl, 6-(1-fluorocyclopropyl)pyridin-3-yl, 6-(2-fluorocyclopropyl)pyridin-3-yl, or 2-cyclopropylpyrimidin-5-yl; or
1H-benzo[d]imidazol-2-yl, 1H-benzo[d]imidazol-4-yl, 1H-benzo[d]imidazol-5-yl, 1H-benzo[d]imidazol-6-yl, 1H-benzo[d]imidazol-7-yl, [1,2,4]triazolo[1,5-a]pyridin-2-yl, [1,2,4]triazolo[1,5-a]pyridin-5-yl, [1,2,4]triazolo[1,5-a]pyridin-6-yl, [1,2,4]triazolo[1,5-a]pyridin-7-yl, [1,2,4]triazolo[1,5-a]pyridin-8-yl, 3H-imidazo[4,5-b]pyridine-2-yl, 3H-imidazo[4,5-b]pyridine-5-yl, 3H-imidazo[4,5-b]pyridine-6-yl, 3H-imidazo[4,5-b]pyridine-7-yl, 1H-imidazo[4,5-b]pyridin-2-yl, 1H-imidazo[4,5-b]pyridin-5-yl, 1H-imidazo[4,5-b]pyridin-6-yl, 1H-imidazo[4,5-b]pyridin-7-yl, benzo[d]oxazol-2-yl, benzo[d]oxazol-4-yl, benzo[d]oxazol-5-yl, benzo[d]oxazol-6-yl, benzo[d]oxazol-7-yl, benzo[d]thiazol-2-yl, benzo[d]thiazol-4-yl, benzo[d]thiazol-5-yl, benzo[d]thiazol-6-yl, benzo[d]thiazol-7-yl, 2-methylbenzo[d]thiazol-6-yl, 5-fluorobenzo[d]thiazol-6-yl, 5-fluoro-2-methylbenzo[d]thiazol-6-yl, 6-fluoro-2-methylbenzo[d]thiazol-5-yl, 7-fluorobenzo[d]thiazol-6-yl, 7-fluoro-2-methylbenzo[d]thiazol-6-yl, 4-fluorobenzo[d]thiazol-5-yl, quinolin-2-yl, quinolin-3-yl, quinolin-4-yl, quinolin-5-yl, quinolin-6-yl, quinolin-7-yl, isoquinolin-1-yl, isoquinolin-3-yl, isoquinolin-4-yl, isoquinolin-5-yl, isoquinolin-6-yl, isoquinolin-7-yl, isoquinolin-8-yl, quinoxalin-2-yl, quinoxalin-3-yl, quinoxalin-4-yl, quinoxalin-5-yl, quinoxalin-6-yl, quinoxalin-7-yl, quinoxalin-8-yl, 1,8-naphthyridin-2-yl, 1,8-naphthyridin-3-yl, 1,8-naphthyridin-4-yl, 2,3-dihydro-[1,4]dioxino[2,3-b]pyridine-6-yl, 2,3-dihydro-[1,4]dioxino[2,3-b]pyridine-7-yl, 2,3-dihydro-[1,4]dioxino[2,3-b]pyridine-8-yl, 3-methylquinoxalin-6-yl, 3-methoxyquinoxalin-6-yl, quinoxalin-6-yl-2,3-d2,1-ethyl-1H-indol-2-yl, 1-methyl-1H-benzo[d]imidazol-2-yl, 1-ethyl-1H-benzo[d]imidazol-2-yl, 1-propyl-1H-benzo[d]imidazol-2-yl, 1-ethyl-5-(trifluoromethyl)-1H-benzo[d]imidazol-2-yl, 1-ethyl-6-(trifluoromethyl)-1H-benzo[d]imidazol-2-yl, 1-ethyl-7-(trifluoromethyl)-1H-benzo[d]imidazol-2-yl, 1-methyl-1H-benzo[d]imidazol-6-yl, 3-ethyl-3H-imidazo[4,5-b]pyridin-2-yl, 1-ethyl-1H-imidazo[4,5-b]pyridin-2-yl, 3-cyclopropylquinoxalin-6-yl,
3-aminoquinoxalin-6-yl, 3-trifluoromethylquinoxalin-6-yl, 3-bifluoromethylquinoxalin-6-yl, 3-(1,1-bifluoroethyl)quinoxalin-6-yl, 2-deuterium-3-methylquinoxalin-6-yl, 2-deuterium-3-methoxyquinoxalin-6-yl, 3-methyl-5-methoxyquinoxalin-6-yl, 3-methyl-7-methoxyquinoxalin-6-yl, 3-methyl-5-trifluoromethylquinoxalin-6-yl, 3-methyl-7-trifluoromethylquinoxalin-6-yl, 1-ethyl-4,5,6,7-tetrahydro-1H-benzo[d]imidazol-2-yl, 2,3-dihydro-[1,4]dioxino[2,3-b]pyridin-6-yl, 2,3-dimethyl-quinoxalin-6-yl, 3-ethyl-quinoxalin-6-yl, 3-chloro-quinoxalin-6-yl, 4-methoxy-quinoxalin-6-yl, 3-(difluoromethyl)quinoxalin-6-yl, 3-(1,1-difluoroethyl)quinoxalin-6-yl, or 3-cyclopropyl-quinoxalin-6-yl; or cyclobutyl, cyclopentyl, cyclohexyl, 2,3-dihydro-1H-inden-1-yl, 2,3-dihydro-1H-inden-2-yl, 1,2,3,4-tetrahydronaphthalen-1-yl, 1,2,3,4-tetrahydronaphthalen-2-yl, or 6,7,8,9-tetrahydro-5H-benzo[7]annulen-5-yl; or
thiazolo[5,4-b]pyridin-5-yl, 2-methylthiazolo[5,4-b]pyridin-5-yl, thiazolo[4,5-b]pyridin-5-yl, 2-methylthiazolo[4,5-b]pyridin-5-yl, thieno[2,3-b]pyridin-6-yl, 2-methylthieno[2,3-b]pyridin-6-yl, 2-methylthieno[3,2-b]pyridin-5-yl, 2-fluorothieno[3,2-b]pyridin-5-yl, or 2-fluorothieno[2,3-b]pyridin-6-y.

In some embodiments, Cy¹ is 2-(trifluoromethoxy)phenyl; 2-methoxyphenyl; 2-(methoxymethyl)phenyl; 2-(trifluoromethyl)phenyl; 4-fluoro-2-(methoxymethyl)phenyl; 4-fluoro-2-methoxyphenyl; 4-fluoro-2-(methoxymethyl)phenyl; 4-fluoro-2-methylphenyl; 2-bromo-4-fluorophenyl; 4-fluoro-2-(methylsulfonyl)phenyl; 4-methyl-2-(trifluoromethyl)phenyl; 2-chloro-4-fluorophenyl; 2,4-difluorophenyl; 2-ethoxy-4-fluorophenyl; 4-fluoro-2-isopropoxyphenyl; 4-fluoro-2-(trifluoromethoxy)phenyl; 2-(difluoromethoxy)-4-fluorophenyl; 2-(difluoromethyl)-4-fluorophenyl; 2-cyclopropyl-4-fluorophenyl; 4-fluoro-2-(1-hydroxyethyl)phenyl; 4-cyclopropyl-2-methoxyphenyl; 2-ethyl-4-fluorophenyl; 4-fluoro-2-(trifluoromethyl)phenyl; 2-methoxy-4-fluorophenyl; 2-(1,1-difluoroethyl)-4-fluorophenyl; 4-fluoro-3-(methoxymethyl)phenyl; 3-methyl-2-(trifluoromethyl)phenyl; 4-fluoro-2,6-dimethoxyphenyl; 2,4-difluoro-6-methoxyphenyl; 2,6-dichloro-4-fluorophenyl; 2,3-dihydrobenzo[b][1,4]dioxin-6-yl; 3,3-dimethyl-2,3-dihydrobenzo[b][1,4]dioxin-6-yl; 2,2-difluorobenzo[d][1,3]dioxol-4-yl; 3-methoxypyridin-2-yl; 2-methoxypyridin-3-yl; 2-(trifluoromethyl)pyridin-3-yl; 6-(difluoromethoxy)pyridin-3-yl; 3-methoxypyridin-4-yl; 5-fluoro-3-(trifluoromethyl)pyridin-2-yl; 5-chloro-1-ethyl-1H-imidazol-2-yl; 1-ethyl-2-(trifluoromethyl)-1H-imidazol-5-yl; 1-ethyl-5-(trifluoromethyl)-1H-pyrazol-4-yl; 5-ethyl-1-methyl-1H-pyrazol-4-yl; 1-methyl-5-(trifluoromethyl)-1H-pyrazol-4-yl; 5-(difluoromethyl)-1-methyl-1H-pyrazol-4-yl; 1-ethyl-3-(trifluoromethyl)-1H-pyrazol-5-yl; 3-chloro-1-ethyl-1H-pyrazol-5-yl; 1-ethyl-4-methyl-1H-pyrazol-5-yl; quinolin-3-yl; quinolin-2-yl; quinoxalin-6-yl; 3-methylquinoxalin-6-yl; 3-methylquinoxalin-6-yl; 3-methylquinoxalin-6-yl; 3-cyclopropylquinoxalin-6-yl; 3-aminoquinoxalin-6-yl; 3-trifluoromethylquinoxalin-6-yl; 3-bifluoromethylquinoxalin-6-yl; 3-(1,1-bifluoroethyl)quinoxalin-6-yl; 2-deuterium-3-methylquinoxalin-6-yl; 2-deuterium-3-methoxyquinoxalin-6-yl; 3-methyl-5-methoxyquinoxalin-6-yl; 3-methyl-7-methoxyquinoxalin-6-yl; 3-methyl-5-trifluoromethylquinoxalin-6-yl; 3-methyl-7-trifluoromethylquinoxalin-6-yl; quinoxalin-6-yl-2,3-d2; 1-ethyl-1H-indol-2-yl; 1-methyl-1H-benzo[d]imidazol-2-yl; 1-ethyl-1H-benzo[d]imidazol-2-yl; 1-propyl-1H-benzo[d]imidazol-2-yl; 1-ethyl-5-(trifluoromethyl)-1H-benzo[d]imidazol-2-yl; 1-ethyl-6-(trifluoromethyl)-1H-benzo[d]imidazol-2-yl; 1-ethyl-7-(trifluoromethyl)-1H-benzo[d]imidazol-2-yl; 1-methyl-1H-benzo[d]imidazol-6-yl; 3-ethyl-3H-imidazo[4,5-b]pyridin-2-yl; 1-ethyl-1H-imidazo[4,5-b]pyridin-2-yl; 1-ethyl-4,5,6,7-tetrahydro-1H-benzo[d]imidazol-2-yl; 3-methoxyquinoxalin-6-yl; 3-(trifluoromethyl)pyridin-4-yl; 4-cyclopropylphenyl; 4-methoxyphenyl; 4-fluorophenyl; 4-cyclopropyl-phenyl; 4-(trifluoromethyl)phenyl; 4-methylphenyl; 4-(difluoromethyl)phenyl; 4-isopropoxyphenyl; 2-fluoro-4-(trifluoromethyl)phenyl; 4-cyclopropyl-2-fluorophenyl; 2,4-difluorophenyl; 4-cyclopropyl-2-fluorophenyl; 4-fluoro-2-(trifluoromethyl)phenyl; 3-methoxy-4-(trifluoromethyl)phenyl; 4-fluoro-3-methoxyphenyl; 2,6-difluorophenyl; 2,6-difluoro-4-methoxyphenyl; 2,5-difluoro-4-methoxyphenyl; naphthalen-2-yl; 3,4-dihydro-2H-benzo[b][1,4]oxazin-6-yl; chroman-4-yl; 1,2,3,4-tetrahydronaphthalen-1-yl; 2,3-dihydro-1H-inden-1-yl; 5-isopropoxypyridin-2-yl; 6-isopropoxypyridin-3-yl; 6-(trifluoromethyl)pyridin-3-yl; 3,5-difluoropyridin-2-yl; 3,5-difluoropyridin-4-yl; 1-ethyl-4-cyano-1H-pyrazol-3-yl; quinolin-2-yl; isoquinolin-3-yl; isoquinolin-6-yl; isoquinolin-7-yl; 1,8-naphthyridin-2-yl; quinoxalin-6-yl; quinoxalin-2-yl; [1,2,4]triazolo[1,5-a]pyridin-7-yl; benzo[d]thiazol-2-yl, benzo[d]thiazol-3-yl, benzo[d]thiazol-5-yl, benzo[d]thiazol-6-yl, benzo[d]thiazol-7-yl, 2-methylbenzo[d]thiazol-6-yl, 2-fluorobenzo[d]thiazol-6-yl, 2-bromobenzo[d]thiazol-6-yl, 2-chlorobenzo[d]thiazol-6-yl, 5-methylbenzo[d]thiazol-6-yl, 5-fluorobenzo[d]thiazol-6-yl, 5-bromobenzo[d]thiazol-6-yl, 5-chlorobenzo[d]thiazol-6-yl; 2,3-dihydro-[1,4]dioxino[2,3-b]pyridin-6-yl; 4-(trifluoromethoxy)phenyl; 4-fluorophenyl; 4-acetamidophenyl; 2-fluoro-4-(trifluoromethyl)phenyl; pyridin-2-yl; pyridin-3-yl; pyridin-4-yl; 5-fluoropyridin-2-yl; pyrazin-2-yl; pyrimidin-2-yl; quinolin-7-yl; isoquinolin-7-yl; quinoxalin-6-yl; quinolin-6-yl; quinolin-7-yl; or pyrimidin-4-yl.

In some embodiments, Cy¹ is 4-fluoro-2-(trifluoromethyl)phenyl, or 2-fluoro-4-(trifluoromethyl)phenyl.

### Aspect 2

In some embodiments, the compound of formula (I) is a compound of formula (V):

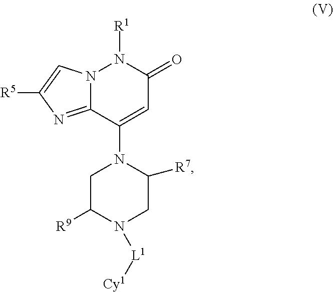

wherein the variables are defined as herein.

In some embodiments, R¹ is methyl, methyl-d3, ethyl, isopropyl, 2-hydroxyethyl, 2-methoxyethyl, 2,2,2-trifluoroethyl, 2,2-difluoroethyl, or cyclopropylmethyl. In some embodiments, R¹ is hydrogen, methyl, ethyl or methyl-d3. In some embodiments, R¹ is methyl or methyl-d3. In some embodiments, R¹ is methyl.

In some embodiments, R⁵ is hydrogen, CN—CH₂—, —CH₂C(O)—OMe, —CH(CH₃)CN, oxiran-2-ylmethyl-, prop-2-yn-1-yl, but-2-yn-1-yl, prop-1-en-2-yl, —CH₂—OH, —CH(CH₃)CN, —CH₂—O—CH₃, —CH(CH₃)—O—CH₃, —CH₂—N(CH₃)₂, —CH═NO—CH₃, —CH₂—S(═O)—CH₃, —CH₂—S(═O)₂—CH₃, or —CH₂—S(═O)(═NH)—CH₃. In some embodiments, R⁵ is CN—CH₂—, prop-2-yn-1-yl, but-2-yn-1-yl, or prop-1-en-2-yl, —CH₂—OH, —CH(CH₃)CN, —CH₂—O—CH₃, —CH(CH₃)—O—CH₃, —CH₂—N(CH₃)₂, —CH═NO—CH₃, —CH₂—S(═O)—CH₃, —CH₂—S(═O)₂—CH₃, or —CH₂—S(═O)(═NH)—CH₃. In some embodiments, R⁵ is CN—CH₂—, —CH₂—OH, —CH(CH₃)CN, —CH₂—O—CH₃, —CH(CH₃)—O—CH₃, —CH₂—N(CH₃)₂, —CH═NO—CH₃, —CH₂—S(═O)—CH₃, —CH₂—S(═O)₂—CH₃, or —CH₂—S(═O)(═NH)—CH₃. In some embodiments, R⁵ is CN—CH₂—.

In some embodiments, R⁷ is methyl; and R⁹ is methyl. In some embodiments, R⁷ is ethyl; and R⁹ is ethyl. In some embodiments, R⁷ is methyl; and R⁹ is ethyl. In some embodiments, R⁷ is ethyl, and R⁹ is methyl.

In some embodiments, L¹ is —CH₂— or —CH(CH₃)—. In some embodiments, L¹ is —CH(CH₃)—.

In some embodiments, Cy¹ is unsubstituted or substituted cycloalkyl, unsubstituted or substituted aryl, unsubstituted or substituted heterocyclyl, or unsubstituted or substituted heteroaryl. In some embodiments, Cy¹ is optionally substituted with one, two or three substituents R^{3a}, wherein R^{3a} is selected from deuterium, fluoro, bromo, chloro, methyl, difluoromethyl, trifluoromethoxy, methoxy, methoxymethyl, trifluoromethyl, methylsulfonyl, difluoro, ethoxy, isopropoxy, trifluoromethoxy, difluoromethoxy, cyclopropyl, 1-hydroxyethyl, ethyl, 1,1-difluoroethyl, cyano, dimethoxy, dichloro, cyclopropyl-, acetamido, 1-methoxyethyl, cyanomethyl, carbamoyl, methoxycarbonyl, dimethylcarbamoyl, (difluoromethoxy)methyl, amino, 1-(difluoromethoxy)ethyl, azetidin-1-yl, 2-methoxypropan-2-yl, 1-methoxycyclopropyl, oxetan-3-yl, 1-methylazetidin-3-yl, or 1-hydroxyazetidin-3-yl.

In some embodiments, Cy¹ is an aryl of from 6 to 14 carbon atoms having a single ring or multiple condensed rings which is unsubstituted or substituted with one, two or three R^{3a}. In some embodiments, said aromatic carbocyclic group is phenyl, naphthyl or anthryl, indanyl, or tetrahydronaphthyl, which is unsubstituted or substituted with one, two or three R^{3a}.

In some embodiments, Cy¹ is phenyl. In some embodiments, Cy¹ is phenyl, which is substituted with one R^{3a} as disclosed herein at position 4 and optionally substituted with R^{3a} on the other position.

In some embodiments, Cy¹ is naphthalenyl. In some embodiments, Cy¹ is naphthalen-1-yl, naphthalen-2-yl, naphthalen-3-yl, naphthalen-4-yl.

In some embodiments, Cy¹ is benzo[d][1,3]dioxol-5-yl, 2,2-dimethylbenzo[d][1,3]dioxol-5-yl, 2,2-dimethylbenzo[d][1,3]dioxol-5-yl, 2,3-dihydrobenzo[b][1,4]dioxin-6-yl, 3,3-dimethyl-2,3-dihydrobenzo[b][1,4]dioxin-6-yl, 3,3-difluoro-2,3-dihydrobenzo[b][1,4]dioxin-6-yl, 2,2-difluorobenzo[d][1,3]dioxol-4-yl, 2,2-dimethylbenzo[d][1,3]dioxol-4-yl, 3,4-dihydro-2H-benzo[b][1,4]oxazin-6-yl, or

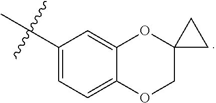 [SMILES: *c1ccc2c(c1)OC1(CC1)CO2]

In some embodiments, Cy¹ is a monocyclic 5- to 9-membered heterocyclyl or a bicyclic 7- to 10-membered heterocyclyl which is unsubstituted or substituted with one, two or three R^{3a}, wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, or heterocyclyl, wherein said alkyl moiety in the group alkyl or alkoxy is unsubstituted or substituted with halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl; preferably R^{3a} is selected from alkoxy, halogen-substituted alkoxy, alkoxyalkyl-, alkyl, halogen-substituted alkyl, halogen, R^{3b}—SO₂—, cycloalkyl, hydroxyalkyl-, cyano, R^{3b}—C(O)—N(R^{3c})—, cyano-substituted alkyl, N(R^{3b}R^{3c})—C(O)—, R^{3b}—O—C(O)—, or heterocyclyl, said cycloalkyl or heterocyclyl is unsubstituted or substituted with alkoxy, alkyl, halogen, or hydroxy, wherein R^{3b} and R^{3c} are each independently hydrogen or alkyl. In some embodiments, said monocyclic 5- to 9-membered heterocyclyl is tetrahydrofuranyl, tetrahydropyranyl, 1,4-dioxanyl, piperidinyl, piperazinyl, or dihydropyridinyl, each of which is unsubstituted or substituted with one, two or three R^{3a} as disclosed herein. In some embodiments, Cy¹ is tetrahydrofuran-2-yl, tetrahydrofuran-3-yl, tetrahydro-2H-pyran-2-yl, tetrahydro-2H-pyran-3-yl, tetrahydro-2H-pyran-4-yl, 1,4-dioxan-2-yl, 1,4-dioxan-3-yl, piperidin-1-yl, piperidin-2-yl, piperidin-3-yl, piperidin-4-yl, piperazin-1-yl, piperazin-2-yl, piperazin-3-yl, piperazin-4-yl, 1,2-dihydropyridin-3-yl, 1,2-dihydropyridin-4-yl, 1,2-dihydropyridin-5-yl, or 1,2-dihydropyridin-6-yl.

In some embodiments, Cy¹ is piperidinyl (e.g., piperidin-1-yl) or piperazinyl (e.g., piperazin-4-yl), which is substituted with one R^{3a} as disclosed herein at position 4 and optionally substituted with R^{3a} on the other position.

In some embodiments, said bicyclic 7- to 10-membered heterocyclyl is chromanyl, preferably chroman-2-yl, chroman-3-yl, or chroman-4-yl.

In some embodiments, Cy¹ is a monocyclic 5- to 9-membered heteroaryl or a bicyclic 7- to 10-membered heteroaryl which is unsubstituted or substituted with one, two or three R^{3a}, wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, or heterocyclyl, wherein said alkyl moiety in the group alkyl or alkoxy is unsubstituted or substituted with halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl; preferably R^{3a} is selected from deuterium, alkoxy, halogen-substituted alkoxy, alkoxyalkyl-, alkyl, halogen-substituted alkyl, halogen, R^{3b}—SO₂—, cycloalkyl, hydroxyalkyl-, cyano, R^{3b}—C(O)—N(R^{3c})—, cyano-substituted alkyl, N(R^{3b}R^{3c})—C(O)—, N(R^{3b}R^{3c})—, R^{3b}—O—C(O)—, or heterocyclyl, said cycloalkyl or heterocyclyl is unsubstituted or substituted with alkoxy, alkyl, halogen, or hydroxy, wherein R^{3b} and R^{3c} are each independently hydrogen or alkyl; more preferably R^{3a} is selected from deuterium, fluoro, bromo, chloro, methyl, difluoromethyl, trifluoromethoxy, methoxy, methoxymethyl, trifluoromethyl, methylsulfonyl, difluoro, ethoxy, isopropoxy, trifluoromethoxy, difluoromethoxy, cyclopropyl, 1-hydroxyethyl, ethyl, 1,1-difluoroethyl, cyano, dimethoxy, dichloro, cyclopropyl-, acetamido, 1-methoxyethyl, cyanomethyl, carbamoyl, methoxycarbonyl, dimethylcarbamoyl, (difluoromethoxy)methyl, amino, 1-(difluoromethoxy)ethyl, azetidin-1-yl, 2-methoxypropan-2-yl, 1-methoxycyclopropyl, oxetan-3-yl, 1-methylazetidin-3-yl, or 1-hydroxyazetidin-3-yl.

In some embodiments, said monocyclic 5- to 9-membered heteroaryl is pyrazolyl, imidazolyl, pyridinyl, pyrimidinyl, pyrazinyl or pyridazinyl, each of which is unsubstituted or substituted with one, two or three R^{3a} as disclosed herein. In some embodiments, said monocyclic 5- to 9-membered heteroaryl is 1H-imidazol-2-yl, 1H-imidazol-4-yl, 1H-imidazol-5-yl, pyridin-2-yl, pyridin-3-yl, pyridin-4-yl, pyrimidin-2-yl, pyrimidin-4-yl, pyrimidin-5-yl, 1H-indol-2-yl, 1H-indol-3-yl, 1H-indol-4-yl, 1H-indol-5-yl, 1H-indol-6-yl, 1H-indol-7-yl, pyrazin-2-yl, or pyridazin-4-yl, each of which is unsubstituted or substituted with one, two or three R^{3a} as disclosed herein.

In some embodiments, said bicyclic 7- to 10-membered heteroaryl is indolyl, benzo[d]imidazolyl, triazolopyridinyl, imidazopyridinyl, benzooxazolyl, benzo[d]thiazolyl, quinolinyl, isoquinolinyl, naphthyridinyl, dioxinopyridineyl, quinoxalinyl, benzo[d]imidazolyl, imidazo[4,5-b]pyridinyl, thiazolo[5,4-b]pyridinyl, thiazolo[4,5-b]pyridinyl, thieno[2,3-b]pyridinyl, or thieno[3,2-b]pyridinyl, each of which is unsubstituted or substituted with one, two or three R^{3a} as disclosed herein. In some embodiments, said bicyclic 7- to 10-membered heteroaryl is 1H-benzo[d]imidazol-2-yl, 1H-benzo[d]imidazol-4-yl, 1H-benzo[d]imidazol-5-yl, 1H-benzo[d]imidazol-6-yl, 1H-benzo[d]imidazol-7-yl, [1,2,4]triazolo[1,5-a]pyridin-2-yl, [1,2,4]triazolo[1,5-a]pyridin-5-yl, [1,2,4]triazolo[1,5-a]pyridin-6-yl, [1,2,4]triazolo[1,5-a]pyridin-7-yl, [1,2,4]triazolo[1,5-a]pyridin-8-yl, 3H-imidazo[4,5-b]pyridine-2-yl, 3H-imidazo[4,5-b]pyridine-5-yl, 3H-imidazo[4,5-b]pyridine-6-yl, 3H-imidazo[4,5-b]pyridine-7-yl, 1H-imidazo[4,5-b]pyridin-2-yl, 1H-imidazo[4,5-b]pyridin-5-yl, 1H-imidazo[4,5-b]pyridin-6-yl, 1H-imidazo[4,5-b]pyridin-7-yl, benzo[d]oxazol-2-yl, benzo[d]oxazol-4-yl, benzo[d]oxazol-5-yl, benzo[d]oxazol-6-yl, benzo[d]oxazol-7-yl, benzo[d]thiazol-2-yl, benzo[d]thiazol-4-yl, benzo[d]thiazol-5-yl, benzo[d]thiazol-6-yl, benzo[d]thiazol-7-yl, quinolin-2-yl, quinolin-3-yl, quinolin-4-yl, quinolin-5-yl, quinolin-6-yl, quinolin-7-yl, isoquinolin-1-yl, isoquinolin-3-yl, isoquinolin-4-yl, isoquinolin-5-yl, isoquinolin-6-yl, isoquinolin-7-yl, isoquinolin-8-yl, quinoxalin-2-yl, quinoxalin-3-yl, quinoxalin-4-yl, quinoxalin-5-yl, quinoxalin-6-yl, quinoxalin-7-yl, quinoxalin-8-yl, 1,8-naphthyridin-2-yl, 1,8-naphthyridin-3-yl, 1,8-naphthyridin-4-yl, 2,3-dihydro-[1,4]dioxino[2,3-b]pyridine-6-yl, 2,3-dihydro-[1,4]dioxino[2,3-b]pyridine-7-yl, 2,3-dihydro-[1,4]dioxino[2,3-b]pyridine-8-yl, quinoxalin-6-yl-2,3-d2, 1H-indol-2-yl, 1H-benzo[d]imidazol-2-yl, 1-methyl-1H-benzo[d]imidazol-6-yl, 3H-imidazo[4,5-b]pyridin-2-yl, 4,5,6,7-tetrahydro-1H-benzo[d]imidazol-2-yl, 2,3-dihydro-[1,4]dioxino[2,3-b]pyridin-6-yl, thiazolo[5,4-b]pyridin-2-yl, thiazolo[5,4-b]pyridin-5-yl, thiazolo[5,4-b]pyridin-6-yl, thiazolo[5,4-b]pyridin-7-yl, thiazolo[4,5-b]pyridin-2-yl, thiazolo[4,5-b]pyridin-5-yl, thiazolo[4,5-b]pyridin-6-yl, thiazolo[4,5-b]pyridin-7-yl, thieno[2,3-b]pyridin-2-yl, thieno[2,3-b]pyridin-3-yl, thieno[2,3-b]pyridin-4-yl, thieno[2,3-b]pyridin-5-yl, thieno[2,3-b]pyridin-6-yl, thieno[3,2-b]pyridin-2-yl, thieno[3,2-b]pyridin-3-yl, thieno[3,2-b]pyridin-5-yl, thieno[3,2-b]pyridin-6-yl, thieno[3,2-b]pyridin-7-yl, each of which is unsubstituted or substituted with one, two or three R^{3a} as disclosed herein.

In some embodiments, Cy¹ is quinoxalinyl, e.g., quinoxalin-2-yl, quinoxalin-3-yl, quinoxalin-4-yl, quinoxalin-5-yl, quinoxalin-6-yl, quinoxalin-7-yl, quinoxalin-8-yl, preferably quinoxalin-6-yl, which is unsubstituted or substituted with one, two or three R^{3a}, wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, or heterocyclyl, wherein said alkyl moiety in the group alkyl or alkoxy is unsubstituted or substituted with halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl; preferably R^{3a} is selected from deuterium, alkoxy, halogen-substituted alkoxy, alkoxyalkyl-, alkyl, halogen-substituted alkyl, halogen, R^{3b}—SO₂—, cycloalkyl, hydroxyalkyl-, cyano, R^{3b}—C(O)—N(R^{3c})—, cyano-substituted alkyl, N(R^{3b}R^{3c})—C(O)—, N(R^{3b}R^{3c})—, R^{3b}—O—C(O)—, or heterocyclyl, said cycloalkyl or heterocyclyl is unsubstituted or substituted with alkoxy, alkyl, halogen, or hydroxy, wherein R^{3b} and R^{3c} are each independently hydrogen or alkyl; more preferably R^{3a} is selected from deuterium, fluoro, bromo, chloro, methyl, difluoromethyl, trifluoromethoxy, methoxy, methoxymethyl, trifluoromethyl, methylsulfonyl, difluoro, ethoxy, isopropoxy, trifluoromethoxy, difluoromethoxy, cyclopropyl, 1-hydroxyethyl, ethyl, 1,1-difluoroethyl, cyano, dimethoxy, dichloro, cyclopropyl-, acetamido, 1-methoxyethyl, cyanomethyl, carbamoyl, methoxycarbonyl, dimethylcarbamoyl, (difluoromethoxy)methyl, amino, 1-(difluoromethoxy)ethyl, azetidin-1-yl, 2-methoxypropan-2-yl, 1-methoxycyclopropyl, oxetan-3-yl, 1-methylazetidin-3-yl, or 1-hydroxyazetidin-3-yl.

In some embodiments, Cy¹ is quinoxalin-6-yl, which is unsubstituted or substituted with one, two or three R^{3a}, wherein R^{3a} is deuterium, methyl, ethyl, isopropyl, cyanomethyl, 2-cyanoethyl, 2-cyanopropan-2-yl, hydroxymethyl, 2-hydroxyethyl, 1-hydroxyethyl, 3-hydroxypropyl, 2-hydroxypropan-2-yl, difluoromethyl, trifluoromethyl, 2-fluoropropan-2-yl, methoxy, isopropoxy, difluoromethoxy, fluoro, chloro, cyano, amino, cyclopropyl, 2,2-difluorocyclopropyl, 1-fluorocyclopropyl, 2-fluorocyclopropyl, or cyclobutyl; preferably deuterium, methyl, ethyl, trifluoromethyl, methoxy, isopropoxy, difluoromethoxy, fluoro, chloro, cyano, amino, or cyclopropyl. In some embodiments, Cy¹ is quinoxalin-6-yl, or 3-methyl-quinoxalin-6-yl.

In some embodiments, Cy¹ is benzo[d]thiazol-5-yl or benzo[d]thiazol-6-yl, each of which is unsubstituted or substituted with one, two or three R^{3a}, wherein R^{3a} is deuterium, methyl, ethyl, isopropyl, cyanomethyl, 2-cyanoethyl, 2-cyanopropan-2-yl, hydroxymethyl, 2-hydroxyethyl, 1-hydroxyethyl, 3-hydroxypropyl, 2-hydroxypropan-2-yl, difluoromethyl, trifluoromethyl, 2-fluoropropan-2-yl, methoxy, isopropoxy, difluoromethoxy, fluoro, chloro, cyano, amino, cyclopropyl, 2,2-difluorocyclopropyl, 1-fluorocyclopropyl, 2-fluorocyclopropyl, or cyclobutyl; preferably deuterium, methyl, ethyl, trifluoromethyl, methoxy, isopropoxy, difluoromethoxy, fluoro, chloro, cyano, amino, or cyclopropyl.

In some embodiments, Cy¹ is thiazolo[5,4-b]pyridin-5-yl, thiazolo[5,4-b]pyridin-6-yl, thiazolo[5,4-b]pyridin-7-yl, thiazolo[4,5-b]pyridin-5-yl, thiazolo[4,5-b]pyridin-6-yl, or thiazolo[4,5-b]pyridin-7-yl, each of which is unsubstituted or substituted with one, two or three R^{3a}, wherein R^{3a} is deuterium, methyl, ethyl, isopropyl, cyanomethyl, 2-cyanoethyl, 2-cyanopropan-2-yl, hydroxymethyl, 2-hydroxyethyl, 1-hydroxyethyl, 3-hydroxypropyl, 2-hydroxypropan-2-yl, difluoromethyl, trifluoromethyl, 2-fluoropropan-2-yl, methoxy, isopropoxy, difluoromethoxy, fluoro, chloro, cyano, amino, cyclopropyl, 2,2-difluorocyclopropyl, 1-fluorocyclopropyl, 2-fluorocyclopropyl, or cyclobutyl; preferably deuterium, methyl, ethyl, trifluoromethyl, methoxy, isopropoxy, difluoromethoxy, fluoro, chloro, cyano, amino, or cyclopropyl.

In some embodiments, Cy¹ is thieno[2,3-b]pyridin-6-yl or thieno[3,2-b]pyridin-5-yl, each of which is unsubstituted or substituted with one, two or three R^{3a}, wherein R^{3a} is deuterium, methyl, ethyl, isopropyl, cyanomethyl, 2-cyanoethyl, 2-cyanopropan-2-yl, hydroxymethyl, 2-hydroxyethyl, 1-hydroxyethyl, 3-hydroxypropyl, 2-hydroxypropan-2-yl, difluoromethyl, trifluoromethyl, 2-fluoropropan-2-yl, methoxy, isopropoxy, difluoromethoxy, fluoro, chloro, cyano, amino, cyclopropyl, 2,2-difluorocyclopropyl, 1-fluorocyclopropyl, 2-fluorocyclopropyl, or cyclobutyl; preferably deuterium, methyl, ethyl, trifluoromethyl, methoxy, isopropoxy, difluoromethoxy, fluoro, chloro, cyano, amino, or cyclopropyl.

In some embodiments, Cy¹ is
phenyl, 2-(trifluoromethoxy)phenyl, 2-methoxyphenyl, 2-(methoxymethyl)phenyl, 2-(trifluoromethyl)phenyl, 4-fluoro-2-(methoxymethyl)phenyl, 4-fluoro-2-methoxyphenyl, 4-fluoro-2-methylphenyl, 2-bromo-4-fluorophenyl, 4-fluoro-2-(methylsulfonyl)phenyl, 4-methyl-2-(trifluoromethyl)phenyl, 2-chloro-4-fluorophenyl, 2,4-difluorophenyl, 2-ethoxy-4-fluorophenyl, 4-fluoro-2-isopropoxyphenyl, 4-fluoro-2-(trifluoromethoxy)phenyl, 2-(difluoromethoxy)-4-fluorophenyl, 2-(difluoromethyl)-4-fluorophenyl, 2-cyclopropyl-4-fluorophenyl, 4-fluoro-2-(1-hydroxyethyl)phenyl, 4-cyclopropyl-2-methoxyphenyl,2-ethyl-4-fluorophenyl, 4-fluoro-2-(trifluoromethyl)phenyl, 2-methoxy-4-fluorophenyl, 2-(1,1-difluoroethyl)-4-fluorophenyl, 2-cyano-4-fluorophenyl, 4-fluoro-3-(methoxymethyl)phenyl, 3-methyl-2-(trifluoromethyl)phenyl, 4-fluoro-2,6-dimethoxyphenyl, 2,4-difluoro-6-methoxyphenyl, 2,6-dichloro-4-fluorophenyl, 4-cyclopropylphenyl, 4-methoxyphenyl, 4-fluorophenyl, 4-cyclopropyl-phenyl, 4-(trifluoromethyl)phenyl, 4-methylphenyl, 4-(difluoromethyl)phenyl, 4-isopropoxyphenyl, 2-fluoro-4-(trifluoromethyl)phenyl, 4-cyclopropyl-2-fluorophenyl, 3-methoxy-4-(trifluoromethyl)phenyl, 4-fluoro-3-methoxyphenyl, 2,6-difluorophenyl, 4-(trifluoromethoxy)phenyl, 4-acetamidophenyl, 4-fluoro-2-(1-methoxyethyl)phenyl, 2-(cyanomethyl)-4-fluorophenyl, 3,4-difluoro-2-(trifluoromethyl)phenyl, 2-carbamoyl-4-fluorophenyl, 2-methoxycarbonyl-4-fluorophenyl, 2-(dimethylcarbamoyl)-4-fluorophenyl, 2-((difluoromethoxy)methyl)-4-fluorophenyl, 2-(1-(difluoromethoxy)ethyl)-4-fluorophenyl, 2-(azetidin-1-yl)-4-fluorophenyl, 4-fluoro-2-(2-methoxypropan-2-yl)phenyl, 3-(trifluoromethyl)phenyl, 4-fluoro-2-(1-methoxycyclopropyl)phenyl, 4-fluoro-2-(oxetan-2-yl)phenyl, 4-fluoro-2-(1-methylazetidin-3-yl)phenyl, 4-fluoro-2-(1-hydroxyazetidin-3-yl)phenyl, 4-cyclopropyl-2-methoxyphenyl; 2-ethyl-4-fluorophenyl; 2-methoxy-4-fluorophenyl; 2,6-difluoro-4-methoxyphenyl; 2,5-difluoro-4-methoxyphenyl; or naphthalen-2-yl; or
benzo[d][1,3]dioxol-5-yl, 2,2-dimethylbenzo[d][1,3]dioxol-5-yl, 2,2-dimethylbenzo[d][1,3]dioxol-5-yl, 2,3-dihydrobenzo[b][1,4]dioxin-6-yl, 3,3-dimethyl-2,3-dihydrobenzo[b][1,4]dioxin-6-yl, 3,3-difluoro-2,3-dihydrobenzo[b][1,4]dioxin-6-yl, 2,2-difluorobenzo[d][1,3]dioxol-4-yl, 2,2-dimethylbenzo[d][1,3]dioxol-4-yl, 3,4-dihydro-2H-benzo[b][1,4]oxazin-6-yl, or

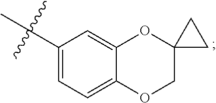 [SMILES: *c1ccc2c(c1)OC1(CC1)CO2]

or
tetrahydrofuran-2-yl, tetrahydrofuran-3-yl, tetrahydro-2H-pyran-2-yl, tetrahydro-2H-pyran-3-yl, tetrahydro-2H-pyran-4-yl, 1,4-dioxan-2-yl, 1,4-dioxan-3-yl, piperidin-2-yl, piperidin-3-yl, piperidin-4-yl, piperazin-2-yl, piperazin-3-yl, 1,2-dihydropyridin-3-yl, 1,2-dihydropyridin-4-yl, 1,2-dihydropyridin-5-yl, or 1,2-dihydropyridin-6-yl; or
chroman-2-yl, chroman-3-yl, or chroman-4-yl; or
1H-pyrazol-3-yl, 1H-pyrazol-4-yl, 1H-pyrazol-5-yl, 1-ethyl-5-(trifluoromethyl)-1H-pyrazol-4-yl; 5-ethyl-1-methyl-1H-pyrazol-4-yl; 1H-imidazol-2-yl, 1H-imidazol-4-yl, 1H-imidazol-5-yl, pyridin-2-yl, pyridin-3-yl, pyridin-4-yl, pyrimidin-2-yl, pyrimidin-4-yl, pyrimidin-5-yl, 1H-indol-2-yl, 1H-indol-3-yl, 1H-indol-4-yl, 1H-indol-5-yl, 1H-indol-6-yl, 1H-indol-7-yl, 3-methoxypyridin-2-yl, 6-isopropoxypyridin-3-yl, 2-methoxypyridin-3-yl, 2-(trifluoromethyl)pyridin-3-yl, 6-(difluoromethoxy)pyridin-3-yl, 3-methoxypyridin-4-yl, 3-(trifluoromethyl)pyridin-4-yl, 5-fluoro-3-(trifluoromethyl)pyridin-2-yl, 5-chloro-1-ethyl-1H-imidazol-2-yl, 1-ethyl-2-(trifluoromethyl)-1H-imidazol-5-yl, 1-ethyl-5-(trifluoromethyl)-1H-pyrazol-4-yl, 5-ethyl-1-methyl-1H-pyrazol-4-yl, 1-methyl-5-(trifluoromethyl)-1H-pyrazol-4-yl, 5-(difluoromethyl)-1-methyl-1H-pyrazol-4-yl, 1-ethyl-3-(trifluoromethyl)-1H-pyrazol-5-yl, 3-chloro-1-ethyl-1H-pyrazol-5-yl, 1-ethyl-4-methyl-1H-pyrazol-5-yl, 5-isopropoxypyridin-2-yl, 6-(trifluoromethyl)pyridin-3-yl, 3,5-difluoropyridin-2-yl, 3,5-difluoropyridin-4-yl, 1-ethyl-4-cyano-1H-pyrazol-3-yl, 5-fluoropyridin-2-yl, pyrazin-2-yl, 3-(trifluoromethyl)pyridazin-4-yl, 6-cyclopropylpyridin-3-yl, 6-cyclobutylpyridin-3-yl, 6-isopropylpyridin-3-yl, 6-cyanopyridin-3-yl, 6-(2-cyanopropan-2-yl)pyridin-3-yl, 6-(2-hydroxypropan-2-yl)pyridin-3-yl, 6-cyclopropyl-2-fluoropyridin-3-yl, 6-methoxypyridin-3-yl, 6-(2-fluoropropan-2-yl)pyridin-3-yl, 5-methoxypyridin-2-yl, 5-cyclopropylpyridin-2-yl, 6-(2,2-difluorocyclopropyl)pyridin-3-yl, 5-(difluoromethyl)pyridin-2-yl, 5-(trifluoromethyl)pyridin-2-yl, 6-(1-fluorocyclopropyl)pyridin-3-yl, 6-(2-fluorocyclopropyl)pyridin-3-yl, or 2-cyclopropylpyrimidin-5-yl; or
1H-benzo[d]imidazol-2-yl, 1H-benzo[d]imidazol-4-yl, 1H-benzo[d]imidazol-5-yl, 1H-benzo[d]imidazol-6-yl, 1H-benzo[d]imidazol-7-yl, [1,2,4]triazolo[1,5-a]pyridin-2-yl, [1,2,4]triazolo[1,5-a]pyridin-5-yl, [1,2,4]triazolo[1,5-a]pyridin-6-yl, [1,2,4]triazolo[1,5-a]pyridin-7-yl, [1,2,4]triazolo[1,5-a]pyridin-8-yl, 3H-imidazo[4,5-b]pyridine-2-yl, 3H-imidazo[4,5-b]pyridine-5-yl, 3H-imidazo[4,5-b]pyridine-6-yl, 3H-imidazo[4,5-b]pyridine-7-yl, 1H-imidazo[4,5-b]pyridin-2-yl, 1H-imidazo[4,5-b]pyridin-5-yl, 1H-imidazo[4,5-b]pyridin-6-yl, 1H-imidazo[4,5-b]pyridin-7-yl, benzo[d]oxazol-2-yl, benzo[d]oxazol-4-yl, benzo[d]oxazol-5-yl, benzo[d]oxazol-6-yl, benzo[d]oxazol-7-yl, benzo[d]thiazol-2-yl, benzo[d]thiazol-4-yl, benzo[d]thiazol-5-yl, benzo[d]thiazol-6-yl, benzo[d]thiazol-7-yl, quinolin-2-yl, quinolin-3-yl, quinolin-4-yl, quinolin-5-yl, quinolin-6-yl, quinolin-7-yl, isoquinolin-1-yl, isoquinolin-3-yl, isoquinolin-4-yl, isoquinolin-5-yl, isoquinolin-6-yl, isoquinolin-7-yl, isoquinolin-8-yl, quinoxalin-2-yl, quinoxalin-3-yl, quinoxalin-4-yl, quinoxalin-5-yl, quinoxalin-6-yl, quinoxalin-7-yl, quinoxalin-8-yl, 1,8-naphthyridin-2-yl, 1,8-naphthyridin-3-yl, 1,8-naphthyridin-4-yl, 2,3-dihydro-[1,4]dioxino[2,3-b]pyridine-6-yl, 2,3-dihydro-[1,4]dioxino[2,3-b]pyridine-7-yl, 2,3-dihydro-[1,4]dioxino[2,3-b]pyridine-8-yl, 3-methylquinoxalin-6-yl, 3-methoxyquinoxalin-6-yl, quinoxalin-6-yl-2,3-d2,1-ethyl-1H-indol-2-yl, 1-methyl-1H-benzo[d]imidazol-2-yl, 1-ethyl-1H-benzo[d]imidazol-2-yl, 1-propyl-1H-benzo[d]imidazol-2-yl, 1-ethyl-5-(trifluoromethyl)-1H-benzo[d]imidazol-2-yl, 1-ethyl-6-(trifluoromethyl)-1H-benzo[d]imidazol-2-yl, 1-ethyl-7-(trifluoromethyl)-1H-benzo[d]imidazol-2-yl, 1-methyl-1H-benzo[d]imidazol-6-yl, 3-ethyl-3H-imidazo[4,5-b]pyridin-2-yl, 1-ethyl-1H-imidazo[4,5-b]pyridin-2-yl, 3-cyclopropylquinoxalin-6-yl, 3-aminoquinoxalin-6-yl, 3-trifluoromethylquinoxalin-6-yl, 3-bifluoromethylquinoxalin-6-yl, 3-(1,1-bifluoroethyl)quinoxalin-6-yl, 2-deuterium-3-methylquinoxalin-6-yl, 2-deuterium-3-methoxyquinoxalin-6-yl, 3-methyl-5-methoxyquinoxalin-6-yl, 3-methyl-7-methoxyquinoxalin-6-yl, 3-methyl-5-trifluoromethylquinoxalin-6-yl, 3-methyl-7-trifluoromethylquinoxalin-6-yl, 1-ethyl-4,5,6,7-tetrahydro-1H-benzo[d]imidazol-2-yl, 2,3-dihydro-[1,4]dioxino[2,3-b]pyridin-6-yl, 2,3-dimethyl-quinoxalin-6-yl, 3-ethyl-quinoxalin-6-yl, 3-chloro-quinoxalin-6-yl, 4-methoxy-quinoxalin-6-yl, 3-(difluoromethyl)quinoxalin-6-yl, 3-(1,1-difluoroethyl)quinoxalin-6-yl, or 3-cyclopropyl-quinoxalin-6-yl; or cyclobutyl, cyclopentyl, cyclohexyl, 2,3-dihydro-1H-inden-1-yl, 2,3-dihydro-1H-inden-2-yl, 1,2,3,4-tetrahydronaphthalen-1-yl, 1,2,3,4-tetrahydronaphthalen-2-yl, or 6,7,8,9-tetrahydro-5H-benzo[7]annulen-5-yl.

In some embodiments, Cy¹ is 2-(trifluoromethoxy)phenyl; 2-methoxyphenyl; 2-(methoxymethyl)phenyl; 2-(trifluoromethyl)phenyl; 4-fluoro-2-(methoxymethyl)phenyl; 4-fluoro-2-methoxyphenyl; 4-fluoro-2-(methoxymethyl)phenyl; 4-fluoro-2-methylphenyl; 2-bromo-4-fluorophenyl; 4-fluoro-2-(methylsulfonyl)phenyl; 4-methyl-2-(trifluoromethyl)phenyl; 2-chloro-4-fluorophenyl; 2,4-difluorophenyl; 2-ethoxy-4-fluorophenyl; 4-fluoro-2-isopropoxyphenyl; 4-fluoro-2-(trifluoromethoxy)phenyl; 2-(difluoromethoxy)-4-fluorophenyl; 2-(difluoromethyl)-4-fluorophenyl; 2-cyclopropyl-4-fluorophenyl; 4-fluoro-2-(1-hydroxyethyl)phenyl; 4-cyclopropyl-2-methoxyphenyl; 2-ethyl-4-fluorophenyl; 4-fluoro-2-(trifluoromethyl)phenyl; 2-methoxy-4-fluorophenyl; 2-(1,1-difluoroethyl)-4-fluorophenyl; 4-fluoro-3-(methoxymethyl)phenyl; 3-methyl-2-(trifluoromethyl)phenyl; 4-fluoro-2,6-dimethoxyphenyl; 2,4-difluoro-6-methoxyphenyl; 2,6-dichloro-4-fluorophenyl; 2,3-dihydrobenzo[b][1,4]dioxin-6-yl; 3,3-dimethyl-2,3-dihydrobenzo[b][1,4]dioxin-6-yl; 2,2-difluorobenzo[d][1,3]dioxol-4-yl; 3-methoxypyridin-2-yl; 2-methoxypyridin-3-yl; 2-(trifluoromethyl)pyridin-3-yl; 6-(difluoromethoxy)pyridin-3-yl; 3-methoxypyridin-4-yl; 5-fluoro-3-(trifluoromethyl)pyridin-2-yl; 5-chloro-1-ethyl-1H-imidazol-2-yl; 1-ethyl-2-(trifluoromethyl)-1H-imidazol-5-yl; 1-ethyl-5-(trifluoromethyl)-1H-pyrazol-4-yl; 5-ethyl-1-methyl-1H-pyrazol-4-yl; 1-methyl-5-(trifluoromethyl)-1H-pyrazol-4-yl; 5-(difluoromethyl)-1-methyl-1H-pyrazol-4-yl; 1-ethyl-3-(trifluoromethyl)-1H-pyrazol-5-yl; 3-chloro-1-ethyl-1H-pyrazol-5-yl; 1-ethyl-4-methyl-1H-pyrazol-5-yl; quinolin-3-yl; quinolin-2-yl; quinoxalin-6-yl; 3-methylquinoxalin-6-yl; 3-methylquinoxalin-6-yl; 3-methylquinoxalin-6-yl; 3-cyclopropylquinoxalin-6-yl; 3-aminoquinoxalin-6-yl; 3-trifluoromethylquinoxalin-6-yl; 3-bifluoromethylquinoxalin-6-yl; 3-(1,1-bifluoroethyl)quinoxalin-6-yl; 2-deuterium-3-methylquinoxalin-6-yl; 2-deuterium-3-methoxyquinoxalin-6-yl; 3-methyl-5-methoxyquinoxalin-6-yl; 3-methyl-7-methoxyquinoxalin-6-yl; 3-methyl-5-trifluoromethylquinoxalin-6-yl; 3-methyl-7-trifluoromethylquinoxalin-6-yl; quinoxalin-6-yl-2,3-d2; 1-ethyl-1H-indol-2-yl; 1-methyl-1H-benzo[d]imidazol-2-yl; 1-ethyl-1H-benzo[d]imidazol-2-yl; 1-propyl-1H-benzo[d]imidazol-2-yl; 1-ethyl-5-(trifluoromethyl)-1H-benzo[d]imidazol-2-yl; 1-ethyl-6-(trifluoromethyl)-1H-benzo[d]imidazol-2-yl; 1-ethyl-7-(trifluoromethyl)-1H-benzo[d]imidazol-2-yl; 1-methyl-1H-benzo[d]imidazol-6-yl; 3-ethyl-3H-imidazo[4,5-b]pyridin-2-yl; 1-ethyl-1H-imidazo[4,5-b]pyridin-2-yl; 1-ethyl-4,5,6,7-tetrahydro-1H-benzo[d]imidazol-2-yl; 3-methoxyquinoxalin-6-yl; 3-(trifluoromethyl)pyridin-4-yl; 4-cyclopropylphenyl; 4-methoxyphenyl; 4-fluorophenyl; 4-cyclopropyl-phenyl; 4-(trifluoromethyl)phenyl; 4-methylphenyl; 4-(difluoromethyl)phenyl; 4-isopropoxyphenyl; 2-fluoro-4-(trifluoromethyl)phenyl; 4-cyclopropyl-2-fluorophenyl; 2,4-difluorophenyl; 4-cyclopropyl-2-fluorophenyl; 4-fluoro-2-(trifluoromethyl)phenyl; 3-methoxy-4-(trifluoromethyl)phenyl; 4-fluoro-3-methoxyphenyl; 2,6-difluorophenyl; 2,6-difluoro-4-methoxyphenyl; 2,5-difluoro-4-methoxyphenyl; naphthalen-2-yl; 3,4-dihydro-2H-benzo[b][1,4]oxazin-6-yl; chroman-4-yl; 1,2,3,4-tetrahydronaphthalen-1-yl; 2,3-dihydro-1H-inden-1-yl; 5-isopropoxypyridin-2-yl; 6-isopropoxypyridin-3-yl; 6-(trifluoromethyl)pyridin-3-yl; 3,5-difluoropyridin-2-yl; 3,5-difluoropyridin-4-yl; 1-ethyl-4-cyano-1H-pyrazol-3-yl; quinolin-2-yl; isoquinolin-3-yl; isoquinolin-6-yl; isoquinolin-7-yl; 1,8-naphthyridin-2-yl; quinoxalin-6-yl; quinoxalin-2-yl; [1,2,4]triazolo[1,5-a]pyridin-7-yl; benzo[d]thiazol-2-yl, benzo[d]thiazol-3-yl, benzo[d]thiazol-5-yl, benzo[d]thiazol-6-yl, benzo[d]thiazol-7-yl, 2-methylbenzo[d]thiazol-6-yl, 2-fluorobenzo[d]thiazol-6-yl, 2-bromobenzo[d]thiazol-6-yl, 2-chlorobenzo[d]thiazol-6-yl, 5-methylbenzo[d]thiazol-6-yl, 5-fluorobenzo[d]thiazol-6-yl, 5-bromobenzo[d]thiazol-6-yl, 5-chlorobenzo[d]thiazol-6-yl; 2,3-dihydro-[1,4]dioxino[2,3-b]pyridin-6-yl; 4-(trifluoromethoxy)phenyl; 4-fluorophenyl; 4-acetamidophenyl; 2-fluoro-4-(trifluoromethyl)phenyl; pyridin-2-yl; pyridin-3-yl; pyridin-4-yl; 5-fluoropyridin-2-yl; pyrazin-2-yl; pyrimidin-2-yl; quinolin-7-yl; isoquinolin-7-yl; quinoxalin-6-yl; quinolin-6-yl; quinolin-7-yl; or pyrimidin-4-yl.

In some embodiments, Cy¹ is 4-fluoro-2-(trifluoromethyl)phenyl, or 2-fluoro-4-(trifluoromethyl)phenyl.

### Aspect 3

In some embodiments, the compound of formula (I) is a compound of formula (VI):

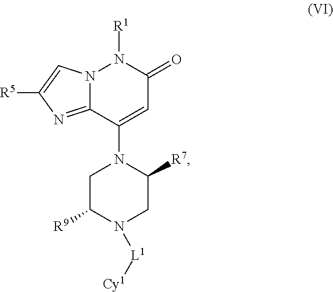

wherein the variables are defined as herein.

In some embodiments, R¹ is methyl, methyl-d3, ethyl, isopropyl, 2-hydroxyethyl, 2-methoxyethyl, 2,2,2-trifluoroethyl, 2,2-difluoroethyl, or cyclopropylmethyl. In some embodiments, R¹ is hydrogen, methyl, ethyl or methyl-d3. In some embodiments, R¹ is methyl or methyl-d3. In some embodiments, R¹ is methyl.

In some embodiments, R⁵ is hydrogen, CN—CH₂—, —CH₂C(O)—OMe, —CH(CH₃)CN, oxiran-2-ylmethyl-, prop-2-yn-1-yl, but-2-yn-1-yl, prop-1-en-2-yl, —CH₂—OH, —CH(CH₃)CN, —CH₂—O—CH₃, —CH(CH₃)—O—CH₃, —CH₂—N(CH₃)₂, —CH═N—O—CH₃, —CH₂—S(═O)—CH₃, —CH₂—S(═O)₂—CH₃, or —CH₂—S(═O)(═NH)—CH₃. In some embodiments, R⁵ is CN—CH₂—, prop-2-yn-1-yl, but-2-yn-1-yl, or prop-1-en-2-yl, —CH₂—OH, —CH(CH₃)CN, —CH₂—O—CH₃, —CH(CH₃)—O—CH₃, —CH₂—N(CH₃)₂, —CH═NO—CH₃, —CH₂—S(═O)—CH₃, —CH₂—S(═O)₂—CH₃, or —CH₂—S(═O)(═NH)—CH₃. In some embodiments, R⁵ is CN—CH₂—, —CH₂—OH, —CH(CH₃)CN, —CH₂—O—CH₃, —CH(CH₃)—O—CH₃, —CH₂—N(CH₃)₂, —CH═N—O—CH₃, —CH₂—S(═O)—CH₃, —CH₂—S(═O)₂—CH₃, or —CH₂—S(═O)(═NH)—CH₃. In some embodiments, R⁵ is CN—CH₂—.

In some embodiments, R⁷ is methyl; and R⁹ is methyl. In some embodiments, R⁷ is ethyl; and R⁹ is ethyl. In some embodiments, R⁷ is methyl; and R⁹ is ethyl. In some embodiments, R⁷ is ethyl, and R⁹ is methyl.

In some embodiments, L¹ is —CH₂— or —CH(CH₃)—. In some embodiments, L¹ is —CH(CH₃)—.

In some embodiments, Cy¹ is unsubstituted or substituted cycloalkyl, unsubstituted or substituted aryl, unsubstituted or substituted heterocyclyl, or unsubstituted or substituted heteroaryl. In some embodiments, Cy¹ is optionally substituted with one, two or three substituents R^{3a}, wherein R^{3a} is selected from deuterium, fluoro, bromo, chloro, methyl, difluoromethyl, trifluoromethoxy, methoxy, methoxymethyl, trifluoromethyl, methylsulfonyl, difluoro, ethoxy, isopropoxy, trifluoromethoxy, difluoromethoxy, cyclopropyl, 1-hydroxyethyl, ethyl, 1,1-difluoroethyl, cyano, dimethoxy, dichloro, cyclopropyl-, acetamido, 1-methoxyethyl, cyanomethyl, carbamoyl, methoxycarbonyl, dimethylcarbamoyl, (difluoromethoxy)methyl, amino, 1-(difluoromethoxy)ethyl, azetidin-1-yl, 2-methoxypropan-2-yl, 1-methoxycyclopropyl, oxetan-3-yl, 1-methylazetidin-3-yl, or 1-hydroxyazetidin-3-yl.

In some embodiments, Cy¹ is an aryl of from 6 to 14 carbon atoms having a single ring or multiple condensed rings which is unsubstituted or substituted with one, two or three R^{3a}. In some embodiments, said aromatic carbocyclic group is phenyl, naphthyl or anthryl, indanyl, or tetrahydronaphthyl, which is unsubstituted or substituted with one, two or three R^{3a}.

In some embodiments, Cy¹ is phenyl. In some embodiments, Cy¹ is phenyl, which is substituted with one R^{3a} as disclosed herein at position 4 and optionally substituted with R^{3a} on the other position.

In some embodiments, Cy¹ is naphthalenyl. In some embodiments, Cy¹ is naphthalen-1-yl, naphthalen-2-yl, naphthalen-3-yl, naphthalen-4-yl.

In some embodiments, Cy¹ is benzo[d][1,3]dioxol-5-yl, 2,2-dimethylbenzo[d][1,3]dioxol-5-yl, 2,2-dimethylbenzo[d][1,3]dioxol-5-yl, 2,3-dihydrobenzo[b][1,4]dioxin-6-yl, 3,3-dimethyl-2,3-dihydrobenzo[b][1,4]dioxin-6-yl, 3,3-difluoro-2,3-dihydrobenzo[b][1,4]dioxin-6-yl, 2,2-difluorobenzo[d][1,3]dioxol-4-yl, 2,2-dimethylbenzo[d][1,3]dioxol-4-yl, 3,4-dihydro-2H-benzo[b][1,4]oxazin-6-yl, or

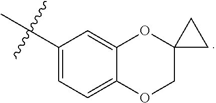 [SMILES: *c1ccc2c(c1)OC1(CC1)CO2]

In some embodiments, Cy¹ is a monocyclic 5- to 9-membered heterocyclyl or a bicyclic 7- to 10-membered heterocyclyl which is unsubstituted or substituted with one, two or three R^{3a}, wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, or heterocyclyl, wherein said alkyl moiety in the group alkyl or alkoxy is unsubstituted or substituted with halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl; preferably R^{3a} is selected from alkoxy, halogen-substituted alkoxy, alkoxyalkyl-, alkyl, halogen-substituted alkyl, halogen, R^{3b}—SO₂—, cycloalkyl, hydroxyalkyl-, cyano, R^{3b}—C(O)—N(R^{3c})—, cyano-substituted alkyl, N(R^{3b}R^{3c})—C(O)—, R^{3b}—O—C(O)—, or heterocyclyl, said cycloalkyl or heterocyclyl is unsubstituted or substituted with alkoxy, alkyl, halogen, or hydroxy, wherein R^{3b} and R^{3c} are each independently hydrogen or alkyl. In some embodiments, said monocyclic 5- to 9-membered heterocyclyl is tetrahydrofuranyl, tetrahydropyranyl, 1,4-dioxanyl, piperidinyl, piperazinyl, or dihydropyridinyl, each of which is unsubstituted or substituted with one, two or three R^{3a} as disclosed herein. In some embodiments, Cy¹ is tetrahydrofuran-2-yl, tetrahydrofuran-3-yl, tetrahydro-2H-pyran-2-yl, tetrahydro-2H-pyran-3-yl, tetrahydro-2H-pyran-4-yl, 1,4-dioxan-2-yl, 1,4-dioxan-3-yl, piperidin-1-yl, piperidin-2-yl, piperidin-3-yl, piperidin-4-yl, piperazin-1-yl, piperazin-2-yl, piperazin-3-yl, piperazin-4-yl, 1,2-dihydropyridin-3-yl, 1,2-dihydropyridin-4-yl, 1,2-dihydropyridin-5-yl, or 1,2-dihydropyridin-6-yl.

In some embodiments, Cy¹ is piperidinyl (e.g., piperidin-1-yl) or piperazinyl (e.g., piperazin-4-yl), which is substituted with one R^{3a} as disclosed herein at position 4 and optionally substituted with R^{3a} on the other position.

In some embodiments, said bicyclic 7- to 10-membered heterocyclyl is chromanyl, preferably chroman-2-yl, chroman-3-yl, or chroman-4-yl.

In some embodiments, Cy¹ is a monocyclic 5- to 9-membered heteroaryl or a bicyclic 7- to 10-membered heteroaryl which is unsubstituted or substituted with one, two or three R^{3a}, wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, or heterocyclyl, wherein said alkyl moiety in the group alkyl or alkoxy is unsubstituted or substituted with halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl; preferably R^{3a} is selected from deuterium, alkoxy, halogen-substituted alkoxy, alkoxyalkyl-, alkyl, halogen-substituted alkyl, halogen, R^{3b}—SO₂—, cycloalkyl, hydroxyalkyl-, cyano, R^{3b}—C(O)—N(R^{3c})—, cyano-substituted alkyl, N(R^{3b}R^{3c})—C(O)—, N(R^{3b}R^{3c})—, R^{3b}—O—C(O)—, or heterocyclyl, said cycloalkyl or heterocyclyl is unsubstituted or substituted with alkoxy, alkyl, halogen, or hydroxy, wherein R^{3b} and R^{3c} are each independently hydrogen or alkyl; more preferably R^{3a} is selected from deuterium, fluoro, bromo, chloro, methyl, difluoromethyl, trifluoromethoxy, methoxy, methoxymethyl, trifluoromethyl, methylsulfonyl, difluoro, ethoxy, isopropoxy, trifluoromethoxy, difluoromethoxy, cyclopropyl, 1-hydroxyethyl, ethyl, 1,1-difluoroethyl, cyano, dimethoxy, dichloro, cyclopropyl-, acetamido, 1-methoxyethyl, cyanomethyl, carbamoyl, methoxycarbonyl, dimethylcarbamoyl, (difluoromethoxy)methyl, amino, 1-(difluoromethoxy)ethyl, azetidin-1-yl, 2-methoxypropan-2-yl, 1-methoxycyclopropyl, oxetan-3-yl, 1-methylazetidin-3-yl, or 1-hydroxyazetidin-3-yl.

In some embodiments, said monocyclic 5- to 9-membered heteroaryl is pyrazolyl, imidazolyl, pyridinyl, pyrimidinyl, pyrazinyl or pyridazinyl, each of which is unsubstituted or substituted with one, two or three R^{3a} as disclosed herein. In some embodiments, said monocyclic 5- to 9-membered heteroaryl is 1H-imidazol-2-yl, 1H-imidazol-4-yl, 1H-imidazol-5-yl, pyridin-2-yl, pyridin-3-yl, pyridin-4-yl, pyrimidin-2-yl, pyrimidin-4-yl, pyrimidin-5-yl, 1H-indol-2-yl, 1H-indol-3-yl, 1H-indol-4-yl, 1H-indol-5-yl, 1H-indol-6-yl, 1H-indol-7-yl, pyrazin-2-yl, or pyridazin-4-yl, each of which is unsubstituted or substituted with one, two or three R^{3a} as disclosed herein.

In some embodiments, said bicyclic 7- to 10-membered heteroaryl is indolyl, benzo[d]imidazolyl, triazolopyridinyl, imidazopyridinyl, benzooxazolyl, benzo[d]thiazolyl, quinolinyl, isoquinolinyl, naphthyridinyl, dioxinopyridineyl, quinoxalinyl, benzo[d]imidazolyl, imidazo[4,5-b]pyridinyl, thiazolo[5,4-b]pyridinyl, thiazolo[4,5-b]pyridinyl, thieno[2,3-b]pyridinyl, or thieno[3,2-b]pyridinyl, each of which is unsubstituted or substituted with one, two or three R^{3a} as disclosed herein. In some embodiments, said bicyclic 7- to 10-membered heteroaryl is 1H-benzo[d]imidazol-2-yl, 1H-benzo[d]imidazol-4-yl, 1H-benzo[d]imidazol-5-yl, 1H-benzo[d]imidazol-6-yl, 1H-benzo[d]imidazol-7-yl, [1,2,4]triazolo[1,5-a]pyridin-2-yl, [1,2,4]triazolo[1,5-a]pyridin-5-yl, [1,2,4]triazolo[1,5-a]pyridin-6-yl, [1,2,4]triazolo[1,5-a]pyridin-7-yl, [1,2,4]triazolo[1,5-a]pyridin-8-yl, 3H-imidazo[4,5-b]pyridine-2-yl, 3H-imidazo[4,5-b]pyridine-5-yl, 3H-imidazo[4,5-b]pyridine-6-yl, 3H-imidazo[4,5-b]pyridine-7-yl, 1H-imidazo[4,5-b]pyridin-2-yl, 1H-imidazo[4,5-b]pyridin-5-yl, 1H-imidazo[4,5-b]pyridin-6-yl, 1H-imidazo[4,5-b]pyridin-7-yl, benzo[d]oxazol-2-yl, benzo[d]oxazol-4-yl, benzo[d]oxazol-5-yl, benzo[d]oxazol-6-yl, benzo[d]oxazol-7-yl, benzo[d]thiazol-2-yl, benzo[d]thiazol-4-yl, benzo[d]thiazol-5-yl, benzo[d]thiazol-6-yl, benzo[d]thiazol-7-yl, quinolin-2-yl, quinolin-3-yl, quinolin-4-yl, quinolin-5-yl, quinolin-6-yl, quinolin-7-yl, isoquinolin-1-yl, isoquinolin-3-yl, isoquinolin-4-yl, isoquinolin-5-yl, isoquinolin-6-yl, isoquinolin-7-yl, isoquinolin-8-yl, quinoxalin-2-yl, quinoxalin-3-yl, quinoxalin-4-yl, quinoxalin-5-yl, quinoxalin-6-yl, quinoxalin-7-yl, quinoxalin-8-yl, 1,8-naphthyridin-2-yl, 1,8-naphthyridin-3-yl, 1,8-naphthyridin-4-yl, 2,3-dihydro-[1,4]dioxino[2,3-b]pyridine-6-yl, 2,3-dihydro-[1,4]dioxino[2,3-b]pyridine-7-yl, 2,3-dihydro-[1,4]dioxino[2,3-b]pyridine-8-yl, quinoxalin-6-yl-2,3-d2, 1H-indol-2-yl, 1H-benzo[d]imidazol-2-yl, 1-methyl-1H-benzo[d]imidazol-6-yl, 3H-imidazo[4,5-b]pyridin-2-yl, 4,5,6,7-tetrahydro-1H-benzo[d]imidazol-2-yl, 2,3-dihydro-[1,4]dioxino[2,3-b]pyridin-6-yl, thiazolo[5,4-b]pyridin-2-yl, thiazolo[5,4-b]pyridin-5-yl, thiazolo[5,4-b]pyridin-6-yl, thiazolo[5,4-b]pyridin-7-yl, thiazolo[4,5-b]pyridin-2-yl, thiazolo[4,5-b]pyridin-5-yl, thiazolo[4,5-b]pyridin-6-yl, thiazolo[4,5-b]pyridin-7-yl, thieno[2,3-b]pyridin-2-yl, thieno[2,3-b]pyridin-3-yl, thieno[2,3-b]pyridin-4-yl, thieno[2,3-b]pyridin-5-yl, thieno[2,3-b]pyridin-6-yl, thieno[3,2-b]pyridin-2-yl, thieno[3,2-b]pyridin-3-yl, thieno[3,2-b]pyridin-5-yl, thieno[3,2-b]pyridin-6-yl, thieno[3,2-b]pyridin-7-yl, each of which is unsubstituted or substituted with one, two or three R^{3a} as disclosed herein.

In some embodiments, Cy¹ is quinoxalinyl, e.g., quinoxalin-2-yl, quinoxalin-3-yl, quinoxalin-4-yl, quinoxalin-5-yl, quinoxalin-6-yl, quinoxalin-7-yl, quinoxalin-8-yl, preferably quinoxalin-6-yl, which is unsubstituted or substituted with one, two or three R^{3a}, wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, or heterocyclyl, wherein said alkyl moiety in the group alkyl or alkoxy is unsubstituted or substituted with halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl; preferably R^{3a} is selected from deuterium, alkoxy, halogen-substituted alkoxy, alkoxyalkyl-, alkyl, halogen-substituted alkyl, halogen, R^{3b}—SO₂—, cycloalkyl, hydroxyalkyl-, cyano, R^{3b}—C(O)—N(R^{3c})—, cyano-substituted alkyl, N(R^{3b}R^{3c})—C(O)—, N(R^{3b}R^{3c})—, R^{3b}—O—C(O)—, or heterocyclyl, said cycloalkyl or heterocyclyl is unsubstituted or substituted with alkoxy, alkyl, halogen, or hydroxy, wherein R^{3b} and R^{3c} are each independently hydrogen or alkyl; more preferably R^{3a} is selected from deuterium, fluoro, bromo, chloro, methyl, difluoromethyl, trifluoromethoxy, methoxy, methoxymethyl, trifluoromethyl, methylsulfonyl, difluoro, ethoxy, isopropoxy, trifluoromethoxy, difluoromethoxy, cyclopropyl, 1-hydroxyethyl, ethyl, 1,1-difluoroethyl, cyano, dimethoxy, dichloro, cyclopropyl-, acetamido, 1-methoxyethyl, cyanomethyl, carbamoyl, methoxycarbonyl, dimethylcarbamoyl, (difluoromethoxy)methyl, amino, 1-(difluoromethoxy)ethyl, azetidin-1-yl, 2-methoxypropan-2-yl, 1-methoxycyclopropyl, oxetan-3-yl, 1-methylazetidin-3-yl, or 1-hydroxyazetidin-3-yl.

In some embodiments, Cy¹ is quinoxalin-6-yl, which is unsubstituted or substituted with one, two or three R^{3a}, wherein R^{3a} is deuterium, methyl, ethyl, isopropyl, cyanomethyl, 2-cyanoethyl, 2-cyanopropan-2-yl, hydroxymethyl, 2-hydroxyethyl, 1-hydroxyethyl, 3-hydroxypropyl, 2-hydroxypropan-2-yl, difluoromethyl, trifluoromethyl, 2-fluoropropan-2-yl, methoxy, isopropoxy, difluoromethoxy, fluoro, chloro, cyano, amino, cyclopropyl, 2,2-difluorocyclopropyl, 1-fluorocyclopropyl, 2-fluorocyclopropyl, or cyclobutyl; preferably deuterium, methyl, ethyl, trifluoromethyl, methoxy, isopropoxy, difluoromethoxy, fluoro, chloro, cyano, amino, or cyclopropyl. In some embodiments, Cy¹ is quinoxalin-6-yl, or 3-methyl-quinoxalin-6-yl.

In some embodiments, Cy¹ is benzo[d]thiazol-5-yl or benzo[d]thiazol-6-yl, each of which is unsubstituted or substituted with one, two or three R^{3a}, wherein R^{3a} is deuterium, methyl, ethyl, isopropyl, cyanomethyl, 2-cyanoethyl, 2-cyanopropan-2-yl, hydroxymethyl, 2-hydroxyethyl, 1-hydroxyethyl, 3-hydroxypropyl, 2-hydroxypropan-2-yl, difluoromethyl, trifluoromethyl, 2-fluoropropan-2-yl, methoxy, isopropoxy, difluoromethoxy, fluoro, chloro, cyano, amino, cyclopropyl, 2,2-difluorocyclopropyl, 1-fluorocyclopropyl, 2-fluorocyclopropyl, or cyclobutyl; preferably deuterium, methyl, ethyl, trifluoromethyl, methoxy, isopropoxy, difluoromethoxy, fluoro, chloro, cyano, amino, or cyclopropyl.

In some embodiments, Cy¹ is thiazolo[5,4-b]pyridin-5-yl, thiazolo[5,4-b]pyridin-6-yl, thiazolo[5,4-b]pyridin-7-yl, thiazolo[4,5-b]pyridin-5-yl, thiazolo[4,5-b]pyridin-6-yl, or thiazolo[4,5-b]pyridin-7-yl, each of which is unsubstituted or substituted with one, two or three R^{3a}, wherein R^{3a} is deuterium, methyl, ethyl, isopropyl, cyanomethyl, 2-cyanoethyl, 2-cyanopropan-2-yl, hydroxymethyl, 2-hydroxyethyl, 1-hydroxyethyl, 3-hydroxypropyl, 2-hydroxypropan-2-yl, difluoromethyl, trifluoromethyl, 2-fluoropropan-2-yl, methoxy, isopropoxy, difluoromethoxy, fluoro, chloro, cyano, amino, cyclopropyl, 2,2-difluorocyclopropyl, 1-fluorocyclopropyl, 2-fluorocyclopropyl, or cyclobutyl; preferably deuterium, methyl, ethyl, trifluoromethyl, methoxy, isopropoxy, difluoromethoxy, fluoro, chloro, cyano, amino, or cyclopropyl.

In some embodiments, Cy¹ is thieno[2,3-b]pyridin-6-yl or thieno[3,2-b]pyridin-5-yl, each of which is unsubstituted or substituted with one, two or three R^{3a}, wherein R^{3a} is deuterium, methyl, ethyl, isopropyl, cyanomethyl, 2-cyanoethyl, 2-cyanopropan-2-yl, hydroxymethyl, 2-hydroxyethyl, 1-hydroxyethyl, 3-hydroxypropyl, 2-hydroxypropan-2-yl, difluoromethyl, trifluoromethyl, 2-fluoropropan-2-yl, methoxy, isopropoxy, difluoromethoxy, fluoro, chloro, cyano, amino, cyclopropyl, 2,2-difluorocyclopropyl, 1-fluorocyclopropyl, 2-fluorocyclopropyl, or cyclobutyl; preferably deuterium, methyl, ethyl, trifluoromethyl, methoxy, isopropoxy, difluoromethoxy, fluoro, chloro, cyano, amino, or cyclopropyl.

In some embodiments, Cy¹ is
phenyl, 2-(trifluoromethoxy)phenyl, 2-methoxyphenyl, 2-(methoxymethyl)phenyl, 2-(trifluoromethyl)phenyl, 4-fluoro-2-(methoxymethyl)phenyl, 4-fluoro-2-methoxyphenyl, 4-fluoro-2-methylphenyl, 2-bromo-4-fluorophenyl, 4-fluoro-2-(methylsulfonyl)phenyl, 4-methyl-2-(trifluoromethyl)phenyl, 2-chloro-4-fluorophenyl, 2,4-difluorophenyl, 2-ethoxy-4-fluorophenyl, 4-fluoro-2-isopropoxyphenyl, 4-fluoro-2-(trifluoromethoxy)phenyl, 2-(difluoromethoxy)-4-fluorophenyl, 2-(difluoromethyl)-4-fluorophenyl, 2-cyclopropyl-4-fluorophenyl, 4-fluoro-2-(1-hydroxyethyl)phenyl, 4-cyclopropyl-2-methoxyphenyl,2-ethyl-4-fluorophenyl, 4-fluoro-2-(trifluoromethyl)phenyl, 2-methoxy-4-fluorophenyl, 2-(1,1-difluoroethyl)-4-fluorophenyl, 2-cyano-4-fluorophenyl, 4-fluoro-3-(methoxymethyl)phenyl, 3-methyl-2-(trifluoromethyl)phenyl, 4-fluoro-2,6-dimethoxyphenyl, 2,4-difluoro-6-methoxyphenyl, 2,6-dichloro-4-fluorophenyl, 4-cyclopropylphenyl, 4-methoxyphenyl, 4-fluorophenyl, 4-cyclopropyl-phenyl, 4-(trifluoromethyl)phenyl, 4-methylphenyl, 4-(difluoromethyl)phenyl, 4-isopropoxyphenyl, 2-fluoro-4-(trifluoromethyl)phenyl, 4-cyclopropyl-2-fluorophenyl, 4-fluoro-2-(trifluoromethyl)phenyl,3-methoxy-4-(trifluoromethyl)phenyl, 4-fluoro-3-methoxyphenyl, 2,6-difluorophenyl, 4-(trifluoromethoxy)phenyl, 4-acetamidophenyl, 2-fluoro-4-(trifluoromethyl)phenyl, 4-fluoro-2-(1-methoxyethyl)phenyl, 2-(cyanomethyl)-4-fluorophenyl, 3,4-difluoro-2-(trifluoromethyl)phenyl, 2-carbamoyl-4-fluorophenyl, 2-methoxycarbonyl-4-fluorophenyl, 2-(dimethylcarbamoyl)-4-fluorophenyl, 2-((difluoromethoxy)methyl)-4-fluorophenyl, 2-(1-(difluoromethoxy)ethyl)-4-fluorophenyl, 2-(azetidin-1-yl)-4-fluorophenyl, 4-fluoro-2-(2-methoxypropan-2-yl)phenyl, 3-(trifluoromethyl)phenyl, 4-fluoro-2-(1-methoxycyclopropyl)phenyl, 4-fluoro-2-(oxetan-2-yl)phenyl, 4-fluoro-2-(1-methylazetidin-3-yl)phenyl, 4-fluoro-2-(1-hydroxyazetidin-3-yl)phenyl, 4-cyclopropyl-2-methoxyphenyl; 2-ethyl-4-fluorophenyl; 4-fluoro-2-(trifluoromethyl)phenyl; 2-methoxy-4-fluorophenyl; 2,6-difluoro-4-methoxyphenyl; 2,5-difluoro-4-methoxyphenyl; 3-methoxy-4-(trifluoromethyl)phenyl or naphthalen-2-yl; or
benzo[d][1,3]dioxol-5-yl, 2,2-dimethylbenzo[d][1,3]dioxol-5-yl, 2,2-dimethylbenzo[d][1,3]dioxol-5-yl, 2,3-dihydrobenzo[b][1,4]dioxin-6-yl, 3,3-dimethyl-2,3-dihydrobenzo[b][1,4]dioxin-6-yl, 3,3-difluoro-2,3-dihydrobenzo[b][1,4]dioxin-6-yl, 2,2-difluorobenzo[d][1,3]dioxol-4-yl, 2,2-dimethylbenzo[d][1,3]dioxol-4-yl, 3,4-dihydro-2H-benzo[b][1,4]oxazin-6-yl, or

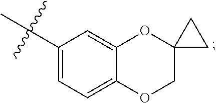 [SMILES: *c1ccc2c(c1)OC1(CC1)CO2]

or
tetrahydrofuran-2-yl, tetrahydrofuran-3-yl, tetrahydro-2H-pyran-2-yl, tetrahydro-2H-pyran-3-yl, tetrahydro-2H-pyran-4-yl, 1,4-dioxan-2-yl, 1,4-dioxan-3-yl, piperidin-2-yl, piperidin-3-yl, piperidin-4-yl, piperazin-2-yl, piperazin-3-yl, 1,2-dihydropyridin-3-yl, 1,2-dihydropyridin-4-yl, 1,2-dihydropyridin-5-yl, or 1,2-dihydropyridin-6-yl; or
chroman-2-yl, chroman-3-yl, or chroman-4-yl; or
1H-pyrazol-3-yl, 1H-pyrazol-4-yl, 1H-pyrazol-5-yl, 1-ethyl-5-(trifluoromethyl)-1H-pyrazol-4-yl; 5-ethyl-1-methyl-1H-pyrazol-4-yl; 1H-imidazol-2-yl, 1H-imidazol-4-yl, 1H-imidazol-5-yl, pyridin-2-yl, pyridin-3-yl, pyridin-4-yl, pyrimidin-2-yl, pyrimidin-4-yl, pyrimidin-5-yl, 1H-indol-2-yl, 1H-indol-3-yl, 1H-indol-4-yl, 1H-indol-5-yl, 1H-indol-6-yl, 1H-indol-7-yl, 3-methoxypyridin-2-yl, 6-isopropoxypyridin-3-yl, 2-methoxypyridin-3-yl, 2-(trifluoromethyl)pyridin-3-yl, 6-(difluoromethoxy)pyridin-3-yl, 3-methoxypyridin-4-yl, 3-(trifluoromethyl)pyridin-4-yl, 5-fluoro-3-(trifluoromethyl)pyridin-2-yl, 5-chloro-1-ethyl-1H-imidazol-2-yl, 1-ethyl-2-(trifluoromethyl)-1H-imidazol-5-yl, 1-ethyl-5-(trifluoromethyl)-1H-pyrazol-4-yl, 5-ethyl-1-methyl-1H-pyrazol-4-yl, 1-methyl-5-(trifluoromethyl)-1H-pyrazol-4-yl, 5-(difluoromethyl)-1-methyl-1H-pyrazol-4-yl, 1-ethyl-3-(trifluoromethyl)-1H-pyrazol-5-yl, 3-chloro-1-ethyl-1H-pyrazol-5-yl, 1-ethyl-4-methyl-1H-pyrazol-5-yl, 5-isopropoxypyridin-2-yl, 6-(trifluoromethyl)pyridin-3-yl, 3,5-difluoropyridin-2-yl, 3,5-difluoropyridin-4-yl, 1-ethyl-4-cyano-1H-pyrazol-3-yl, 5-fluoropyridin-2-yl, pyrazin-2-yl, 3-(trifluoromethyl)pyridazin-4-yl, 6-cyclopropylpyridin-3-yl, 6-cyclobutylpyridin-3-yl, 6-isopropylpyridin-3-yl, 6-cyanopyridin-3-yl, 6-(2-cyanopropan-2-yl)pyridin-3-yl, 6-(2-hydroxypropan-2-yl)pyridin-3-yl, 6-cyclopropyl-2-fluoropyridin-3-yl, 6-methoxypyridin-3-yl, 6-(2-fluoropropan-2-yl)pyridin-3-yl, 5-methoxypyridin-2-yl, 5-cyclopropylpyridin-2-yl, 6-(2,2-difluorocyclopropyl)pyridin-3-yl, 5-(difluoromethyl)pyridin-2-yl, 5-(trifluoromethyl)pyridin-2-yl, 6-(1-fluorocyclopropyl)pyridin-3-yl, 6-(2-fluorocyclopropyl)pyridin-3-yl, or 2-cyclopropylpyrimidin-5-yl; or
1H-benzo[d]imidazol-2-yl, 1H-benzo[d]imidazol-4-yl, 1H-benzo[d]imidazol-5-yl, 1H-benzo[d]imidazol-6-yl, 1H-benzo[d]imidazol-7-yl, [1,2,4]triazolo[1,5-a]pyridin-2-yl, [1,2,4]triazolo[1,5-a]pyridin-5-yl, [1,2,4]triazolo[1,5-a]pyridin-6-yl, [1,2,4]triazolo[1,5-a]pyridin-7-yl, [1,2,4]triazolo[1,5-a]pyridin-8-yl, 3H-imidazo[4,5-b]pyridine-2-yl, 3H-imidazo[4,5-b]pyridine-5-yl, 3H-imidazo[4,5-b]pyridine-6-yl, 3H-imidazo[4,5-b]pyridine-7-yl, 1H-imidazo[4,5-b]pyridin-2-yl, 1H-imidazo[4,5-b]pyridin-5-yl, 1H-imidazo[4,5-b]pyridin-6-yl, 1H-imidazo[4,5-b]pyridin-7-yl, benzo[d]oxazol-2-yl, benzo[d]oxazol-4-yl, benzo[d]oxazol-5-yl, benzo[d]oxazol-6-yl, benzo[d]oxazol-7-yl, benzo[d]thiazol-2-yl, benzo[d]thiazol-4-yl, benzo[d]thiazol-5-yl, benzo[d]thiazol-6-yl, benzo[d]thiazol-7-yl, quinolin-2-yl, quinolin-3-yl, quinolin-4-yl, quinolin-5-yl, quinolin-6-yl, quinolin-7-yl, isoquinolin-1-yl, isoquinolin-3-yl, isoquinolin-4-yl, isoquinolin-5-yl, isoquinolin-6-yl, isoquinolin-7-yl, isoquinolin-8-yl, quinoxalin-2-yl, quinoxalin-3-yl, quinoxalin-4-yl, quinoxalin-5-yl, quinoxalin-6-yl, quinoxalin-7-yl, quinoxalin-8-yl, 1,8-naphthyridin-2-yl, 1,8-naphthyridin-3-yl, 1,8-naphthyridin-4-yl, 2,3-dihydro-[1,4]dioxino[2,3-b]pyridine-6-yl, 2,3-dihydro-[1,4]dioxino[2,3-b]pyridine-7-yl, 2,3-dihydro-[1,4]dioxino[2,3-b]pyridine-8-yl, 3-methylquinoxalin-6-yl, 3-methoxyquinoxalin-6-yl, quinoxalin-6-yl-2,3-d2,1-ethyl-1H-indol-2-yl, 1-methyl-1H-benzo[d]imidazol-2-yl, 1-ethyl-1H-benzo[d]imidazol-2-yl, 1-propyl-1H-benzo[d]imidazol-2-yl, 1-ethyl-5-(trifluoromethyl)-1H-benzo[d]imidazol-2-yl, 1-ethyl-6-(trifluoromethyl)-1H-benzo[d]imidazol-2-yl, 1-ethyl-7-(trifluoromethyl)-1H-benzo[d]imidazol-2-yl, 1-methyl-1H-benzo[d]imidazol-6-yl, 3-ethyl-3H-imidazo[4,5-b]pyridin-2-yl, 1-ethyl-1H-imidazo[4,5-b]pyridin-2-yl, 3-cyclopropylquinoxalin-6-yl, 3-aminoquinoxalin-6-yl, 3-trifluoromethylquinoxalin-6-yl, 3-bifluoromethylquinoxalin-6-yl, 3-(1,1-bifluoroethyl)quinoxalin-6-yl, 2-deuterium-3-methylquinoxalin-6-yl, 2-deuterium-3-methoxyquinoxalin-6-yl, 3-methyl-5-methoxyquinoxalin-6-yl, 3-methyl-7-methoxyquinoxalin-6-yl, 3-methyl-5-trifluoromethylquinoxalin-6-yl, 3-methyl-7-trifluoromethylquinoxalin-6-yl, 1-ethyl-4,5,6,7-tetrahydro-1H-benzo[d]imidazol-2-yl, 2,3-dihydro-[1,4]dioxino[2,3-b]pyridin-6-yl, 2,3-dimethyl-quinoxalin-6-yl, 3-ethyl-quinoxalin-6-yl, 3-chloro-quinoxalin-6-yl, 4-methoxy-quinoxalin-6-yl, 3-(difluoromethyl)quinoxalin-6-yl, 3-(1,1-difluoroethyl)quinoxalin-6-yl, or 3-cyclopropyl-quinoxalin-6-yl; or cyclobutyl, cyclopentyl, cyclohexyl, 2,3-dihydro-1H-inden-1-yl, 2,3-dihydro-1H-inden-2-yl, 1,2,3,4-tetrahydronaphthalen-1-yl, 1,2,3,4-tetrahydronaphthalen-2-yl, or 6,7,8,9-tetrahydro-5H-benzo[7]annulen-5-yl.

In some embodiments, Cy¹ is 2-(trifluoromethoxy)phenyl; 2-methoxyphenyl; 2-(methoxymethyl)phenyl; 2-(trifluoromethyl)phenyl; 4-fluoro-2-(methoxymethyl)phenyl; 4-fluoro-2-methoxyphenyl; 4-fluoro-2-(methoxymethyl)phenyl; 4-fluoro-2-methylphenyl; 2-bromo-4-fluorophenyl; 4-fluoro-2-(methylsulfonyl)phenyl; 4-methyl-2-(trifluoromethyl)phenyl; 2-chloro-4-fluorophenyl; 2,4-difluorophenyl; 2-ethoxy-4-fluorophenyl; 4-fluoro-2-isopropoxyphenyl; 4-fluoro-2-(trifluoromethoxy)phenyl; 2-(difluoromethoxy)-4-fluorophenyl; 2-(difluoromethyl)-4-fluorophenyl; 2-cyclopropyl-4-fluorophenyl; 4-fluoro-2-(1-hydroxyethyl)phenyl; 4-cyclopropyl-2-methoxyphenyl; 2-ethyl-4-fluorophenyl; 4-fluoro-2-(trifluoromethyl)phenyl; 2-methoxy-4-fluorophenyl; 2-(1,1-difluoroethyl)-4-fluorophenyl; 4-fluoro-3-(methoxymethyl)phenyl; 3-methyl-2-(trifluoromethyl)phenyl; 4-fluoro-2,6-dimethoxyphenyl; 2,4-difluoro-6-methoxyphenyl; 2,6-dichloro-4-fluorophenyl; 2,3-dihydrobenzo[b][1,4]dioxin-6-yl; 3,3-dimethyl-2,3-dihydrobenzo[b][1,4]dioxin-6-yl; 2,2-difluorobenzo[d][1,3]dioxol-4-yl; 3-methoxypyridin-2-yl; 2-methoxypyridin-3-yl; 2-(trifluoromethyl)pyridin-3-yl; 6-(difluoromethoxy)pyridin-3-yl; 3-methoxypyridin-4-yl; 5-fluoro-3-(trifluoromethyl)pyridin-2-yl; 5-chloro-1-ethyl-1H-imidazol-2-yl; 1-ethyl-2-(trifluoromethyl)-1H-imidazol-5-yl; 1-ethyl-5-(trifluoromethyl)-1H-pyrazol-4-yl; 5-ethyl-1-methyl-1H-pyrazol-4-yl; 1-methyl-5-(trifluoromethyl)-1H-pyrazol-4-yl; 5-(difluoromethyl)-1-methyl-1H-pyrazol-4-yl; 1-ethyl-3-(trifluoromethyl)-1H-pyrazol-5-yl; 3-chloro-1-ethyl-1H-pyrazol-5-yl; 1-ethyl-4-methyl-1H-pyrazol-5-yl; quinolin-3-yl; quinolin-2-yl; quinoxalin-6-yl; 3-methylquinoxalin-6-yl; 3-methylquinoxalin-6-yl; 3-methylquinoxalin-6-yl; 3-cyclopropylquinoxalin-6-yl; 3-aminoquinoxalin-6-yl; 3-trifluoromethylquinoxalin-6-yl; 3-bifluoromethylquinoxalin-6-yl; 3-(1,1-bifluoroethyl)quinoxalin-6-yl; 2-deuterium-3-methylquinoxalin-6-yl; 2-deuterium-3-methoxyquinoxalin-6-yl; 3-methyl-5-methoxyquinoxalin-6-yl; 3-methyl-7-methoxyquinoxalin-6-yl; 3-methyl-5-trifluoromethylquinoxalin-6-yl; 3-methyl-7-trifluoromethylquinoxalin-6-yl; quinoxalin-6-yl-2,3-d2; 1-ethyl-1H-indol-2-yl; 1-methyl-1H-benzo[d]imidazol-2-yl; 1-ethyl-1H-benzo[d]imidazol-2-yl; 1-propyl-1H-benzo[d]imidazol-2-yl; 1-ethyl-5-(trifluoromethyl)-1H-benzo[d]imidazol-2-yl; 1-ethyl-6-(trifluoromethyl)-1H-benzo[d]imidazol-2-yl; 1-ethyl-7-(trifluoromethyl)-1H-benzo[d]imidazol-2-yl; 1-methyl-1H-benzo[d]imidazol-6-yl; 3-ethyl-3H-imidazo[4,5-b]pyridin-2-yl; 1-ethyl-1H-imidazo[4,5-b]pyridin-2-yl; 1-ethyl-4,5,6,7-tetrahydro-1H-benzo[d]imidazol-2-yl; 3-methoxyquinoxalin-6-yl; 3-(trifluoromethyl)pyridin-4-yl; 4-cyclopropylphenyl; 4-methoxyphenyl; 4-fluorophenyl; 4-cyclopropyl-phenyl; 4-(trifluoromethyl)phenyl; 4-methylphenyl; 4-(difluoromethyl)phenyl; 4-isopropoxyphenyl; 2-fluoro-4-(trifluoromethyl)phenyl; 4-cyclopropyl-2-fluorophenyl; 2,4-difluorophenyl; 4-cyclopropyl-2-fluorophenyl; 4-fluoro-2-(trifluoromethyl)phenyl; 3-methoxy-4-(trifluoromethyl)phenyl; 4-fluoro-3-methoxyphenyl; 2,6-difluorophenyl; 2,6-difluoro-4-methoxyphenyl; 2,5-difluoro-4-methoxyphenyl; naphthalen-2-yl; 3,4-dihydro-2H-benzo[b][1,4]oxazin-6-yl; chroman-4-yl; 1,2,3,4-tetrahydronaphthalen-1-yl; 2,3-dihydro-1H-inden-1-yl; 5-isopropoxypyridin-2-yl; 6-isopropoxypyridin-3-yl; 6-(trifluoromethyl)pyridin-3-yl; 3,5-difluoropyridin-2-yl; 3,5-difluoropyridin-4-yl; 1-ethyl-4-cyano-1H-pyrazol-3-yl; quinolin-2-yl; isoquinolin-3-yl; isoquinolin-6-yl; isoquinolin-7-yl; 1,8-naphthyridin-2-yl; quinoxalin-6-yl; quinoxalin-2-yl; [1,2,4]triazolo[1,5-a]pyridin-7-yl; benzo[d]thiazol-2-yl, benzo[d]thiazol-3-yl, benzo[d]thiazol-5-yl, benzo[d]thiazol-6-yl, benzo[d]thiazol-7-yl, 2-methylbenzo[d]thiazol-6-yl, 2-fluorobenzo[d]thiazol-6-yl, 2-bromobenzo[d]thiazol-6-yl, 2-chlorobenzo[d]thiazol-6-yl, 5-methylbenzo[d]thiazol-6-yl, 5-fluorobenzo[d]thiazol-6-yl, 5-bromobenzo[d]thiazol-6-yl, 5-chlorobenzo[d]thiazol-6-yl; 2,3-dihydro-[1,4]dioxino[2,3-b]pyridin-6-yl; 4-(trifluoromethoxy)phenyl; 4-fluorophenyl; 4-acetamidophenyl; 2-fluoro-4-(trifluoromethyl)phenyl; pyridin-2-yl; pyridin-3-yl; pyridin-4-yl; 5-fluoropyridin-2-yl; pyrazin-2-yl; pyrimidin-2-yl; quinolin-7-yl; isoquinolin-7-yl; quinoxalin-6-yl; quinolin-6-yl; quinolin-7-yl; or pyrimidin-4-yl.

In some embodiments, Cy¹ is 4-fluoro-2-(trifluoromethyl)phenyl, or 2-fluoro-4-(trifluoromethyl)phenyl.

### Aspect 4

In some embodiments, the compound of formula (I) is a compound of formula (VI) as described above.

In some embodiments, R¹ is methyl, methyl-d3, ethyl, isopropyl, 2-hydroxyethyl, 2-methoxyethyl, 2,2,2-trifluoroethyl, 2,2-difluoroethyl, or cyclopropylmethyl. In some embodiments, R¹ is hydrogen, methyl, ethyl or methyl-d3. In some embodiments, R¹ is methyl or methyl-d3. In some embodiments, R¹ is methyl.

In some embodiments, R⁵ is hydrogen, CN—CH₂—, —CH₂C(O)—OMe, —CH(CH₃)CN, oxiran-2-ylmethyl-, prop-2-yn-1-yl, but-2-yn-1-yl, prop-1-en-2-yl, —CH₂—OH, —CH(CH₃)CN, —CH₂—O—CH₃, —CH(CH₃)—O—CH₃, —CH₂—N(CH₃)₂, —CH═N—O—CH₃, —CH₂—S(═O)—CH₃, —CH₂—S(═O)₂—CH₃, or —CH₂—S(═O)(═NH)—CH₃. In some embodiments, R⁵ is CN—CH₂—, prop-2-yn-1-yl, but-2-yn-1-yl, or prop-1-en-2-yl, —CH₂—OH, —CH(CH₃)CN, —CH₂—O—CH₃, —CH(CH₃)—O—CH₃, —CH₂—N(CH₃)₂, —CH═NO—CH₃, —CH₂—S(═O)—CH₃, —CH₂—S(═O)₂—CH₃, or —CH₂—S(═O)(═NH)—CH₃. In some embodiments, R⁵ is CN—CH₂—, —CH₂—OH, —CH(CH₃)CN, —CH₂—O—CH₃, —CH(CH₃)—O—CH₃, —CH₂—N(CH₃)₂, —CH═N—O—CH₃, —CH₂—S(═O)—CH₃, —CH₂—S(═O)₂—CH₃, or —CH₂—S(═O)(═NH)—CH₃. In some embodiments, R⁵ is CN—CH₂—.

In some embodiments, R⁷ is methyl; and R⁹ is methyl. In some embodiments, R⁷ is ethyl; and R⁹ is ethyl. In some embodiments, R⁷ is methyl; and R⁹ is ethyl. In some embodiments, R⁷ is ethyl, and R⁹ is methyl.

In some embodiments, L¹ is —CH₂— or —CH(CH₃)—. In some embodiments, L¹ is —CH(CH₃)—.

In some embodiments, Cy¹ is 2-(trifluoromethoxy)phenyl; 2-methoxyphenyl; 2-(methoxymethyl)phenyl; 2-(trifluoromethyl)phenyl; 4-fluoro-2-(methoxymethyl)phenyl; 4-fluoro-2-methoxyphenyl; 4-fluoro-2-(methoxymethyl)phenyl; 4-fluoro-2-methylphenyl; 2-bromo-4-fluorophenyl; 4-fluoro-2-(methylsulfonyl)phenyl; 4-methyl-2-(trifluoromethyl)phenyl; 2-chloro-4-fluorophenyl; 2,4-difluorophenyl; 2-ethoxy-4-fluorophenyl; 4-fluoro-2-isopropoxyphenyl; 4-fluoro-2-(trifluoromethoxy)phenyl; 2-(difluoromethoxy)-4-fluorophenyl; 2-(difluoromethyl)-4-fluorophenyl; 2-cyclopropyl-4-fluorophenyl; 4-fluoro-2-(1-hydroxyethyl)phenyl; 4-cyclopropyl-2-methoxyphenyl; 2-ethyl-4-fluorophenyl; 4-fluoro-2-(trifluoromethyl)phenyl; 2-methoxy-4-fluorophenyl; 2-(1,1-difluoroethyl)-4-fluorophenyl; 4-fluoro-3-(methoxymethyl)phenyl; 3-methyl-2-(trifluoromethyl)phenyl; 4-fluoro-2,6-dimethoxyphenyl; 2,4-difluoro-6-methoxyphenyl; 2,6-dichloro-4-fluorophenyl; 2,3-dihydrobenzo[b][1,4]dioxin-6-yl; 3,3-dimethyl-2,3-dihydrobenzo[b][1,4]dioxin-6-yl; 2,2-difluorobenzo[d][1,3]dioxol-4-yl; 3-methoxypyridin-2-yl; 2-methoxypyridin-3-yl; 2-(trifluoromethyl)pyridin-3-yl; 6-(difluoromethoxy)pyridin-3-yl; 3-methoxypyridin-4-yl; 5-fluoro-3-(trifluoromethyl)pyridin-2-yl; 5-chloro-1-ethyl-1H-imidazol-2-yl; 1-ethyl-2-(trifluoromethyl)-1H-imidazol-5-yl; 1-ethyl-5-(trifluoromethyl)-1H-pyrazol-4-yl; 5-ethyl-1-methyl-1H-pyrazol-4-yl; 1-methyl-5-(trifluoromethyl)-1H-pyrazol-4-yl; 5-(difluoromethyl)-1-methyl-1H-pyrazol-4-yl; 1-ethyl-3-(trifluoromethyl)-1H-pyrazol-5-yl; 3-chloro-1-ethyl-1H-pyrazol-5-yl; 1-ethyl-4-methyl-1H-pyrazol-5-yl; quinolin-3-yl; quinolin-2-yl; quinoxalin-6-yl; 3-methylquinoxalin-6-yl; 3-methylquinoxalin-6-yl; 3-methylquinoxalin-6-yl; 3-cyclopropylquinoxalin-6-yl; 3-aminoquinoxalin-6-yl; 3-trifluoromethylquinoxalin-6-yl; 3-bifluoromethylquinoxalin-6-yl; 3-(1,1-bifluoroethyl)quinoxalin-6-yl; 2-deuterium-3-methylquinoxalin-6-yl; 2-deuterium-3-methoxyquinoxalin-6-yl; 3-methyl-5-methoxyquinoxalin-6-yl; 3-methyl-7-methoxyquinoxalin-6-yl; 3-methyl-5-trifluoromethylquinoxalin-6-yl; 3-methyl-7-trifluoromethylquinoxalin-6-yl; quinoxalin-6-yl-2,3-d2; 1-ethyl-1H-indol-2-yl; 1-methyl-1H-benzo[d]imidazol-2-yl; 1-ethyl-1H-benzo[d]imidazol-2-yl; 1-propyl-1H-benzo[d]imidazol-2-yl; 1-ethyl-5-(trifluoromethyl)-1H-benzo[d]imidazol-2-yl; 1-ethyl-6-(trifluoromethyl)-1H-benzo[d]imidazol-2-yl; 1-ethyl-7-(trifluoromethyl)-1H-benzo[d]imidazol-2-yl; 1-methyl-1H-benzo[d]imidazol-6-yl; 3-ethyl-3H-imidazo[4,5-b]pyridin-2-yl; 1-ethyl-1H-imidazo[4,5-b]pyridin-2-yl; 1-ethyl-4,5,6,7-tetrahydro-1H-benzo[d]imidazol-2-yl; 3-methoxyquinoxalin-6-yl; 3-(trifluoromethyl)pyridin-4-yl; 4-cyclopropylphenyl; 4-methoxyphenyl; 4-fluorophenyl; 4-cyclopropyl-phenyl; 4-(trifluoromethyl)phenyl; 4-methylphenyl; 4-(difluoromethyl)phenyl; 4-isopropoxyphenyl; 2-fluoro-4-(trifluoromethyl)phenyl; 4-cyclopropyl-2-fluorophenyl; 2,4-difluorophenyl; 4-cyclopropyl-2-fluorophenyl; 4-fluoro-2-(trifluoromethyl)phenyl; 3-methoxy-4-(trifluoromethyl)phenyl; 4-fluoro-3-methoxyphenyl; 2,6-difluorophenyl; 2,6-difluoro-4-methoxyphenyl; 2,5-difluoro-4-methoxyphenyl; naphthalen-2-yl; 3,4-dihydro-2H-benzo[b][1,4]oxazin-6-yl; chroman-4-yl; 1,2,3,4-tetrahydronaphthalen-1-yl; 2,3-dihydro-1H-inden-1-yl; 5-isopropoxypyridin-2-yl; 6-isopropoxypyridin-3-yl; 6-(trifluoromethyl)pyridin-3-yl; 3,5-difluoropyridin-2-yl; 3,5-difluoropyridin-4-yl; 1-ethyl-4-cyano-1H-pyrazol-3-yl; quinolin-2-yl; isoquinolin-3-yl; isoquinolin-6-yl; isoquinolin-7-yl; 1,8-naphthyridin-2-yl; quinoxalin-6-yl; quinoxalin-2-yl; [1,2,4]triazolo[1,5-a]pyridin-7-yl; benzo[d]thiazol-2-yl, benzo[d]thiazol-3-yl, benzo[d]thiazol-5-yl, benzo[d]thiazol-6-yl, benzo[d]thiazol-7-yl, 2-methylbenzo[d]thiazol-6-yl, 2-fluorobenzo[d]thiazol-6-yl, 2-bromobenzo[d]thiazol-6-yl, 2-chlorobenzo[d]thiazol-6-yl, 5-methylbenzo[d]thiazol-6-yl, 5-fluorobenzo[d]thiazol-6-yl, 5-bromobenzo[d]thiazol-6-yl, 5-chlorobenzo[d]thiazol-6-yl; 2,3-dihydro-[1,4]dioxino[2,3-b]pyridin-6-yl; 4-(trifluoromethoxy)phenyl; 4-fluorophenyl; 4-acetamidophenyl; 2-fluoro-4-(trifluoromethyl)phenyl; pyridin-2-yl; pyridin-3-yl; pyridin-4-yl; 5-fluoropyridin-2-yl; pyrazin-2-yl; pyrimidin-2-yl; quinolin-7-yl; isoquinolin-7-yl; quinoxalin-6-yl; quinolin-6-yl; quinolin-7-yl; or pyrimidin-4-yl.

In some embodiments, Cy¹ is 4-fluoro-2-(trifluoromethyl)phenyl, or 2-fluoro-4-(trifluoromethyl)phenyl.

### General Synthetic Schemes

Compounds disclosed herein, including salts thereof, can be prepared using known organic synthesis techniques and can be synthesized according to any of numerous possible synthetic routes.

The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from room temperature to the solvent's boiling temperature. A given reaction can be carried out in one solvent or mixture of solvents.

The selection of appropriate protecting group, can be readily determined by one skilled in the art.

Reactions can be monitored according to any suitable method known in the art, such as NMR, UV, HPLC, LC-MS and TLC. Compounds can be purified by a variety of methods, including HPLC and normal phase silica flash column chromatography.

Chiral analytic HPLC was used for the retention time analysis of different chiral examples, the conditions were divided into the methods as below according to the column, mobile phase, solvent ration used. Preparation of homochiral examples may be carried out by techniques known to one skilled in the art. The absolute stereochemistry was not assigned at the newly formed carbon-nitrogen bond.

The compounds disclosed herein can be prepared by following Scheme I, II.

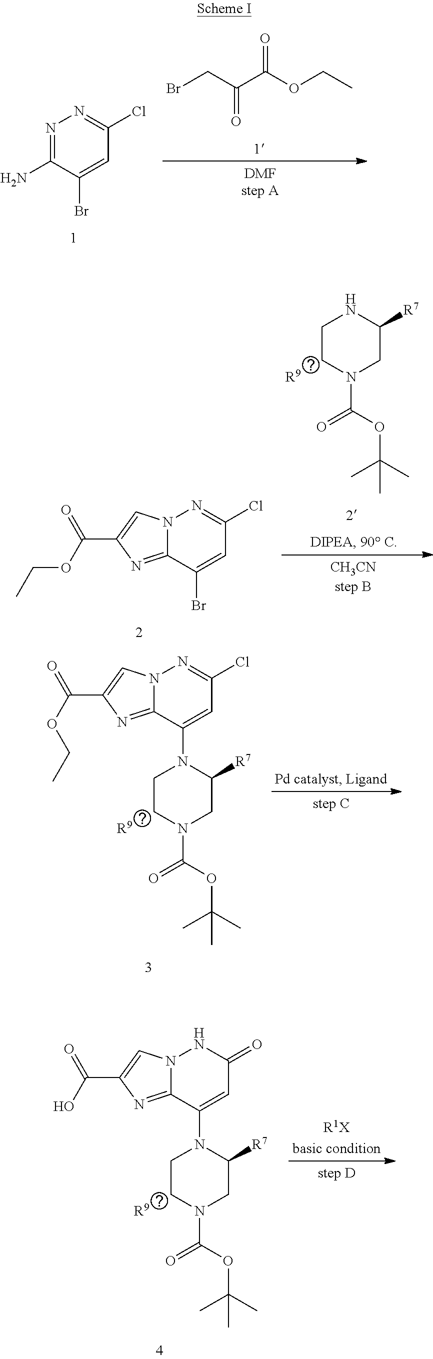

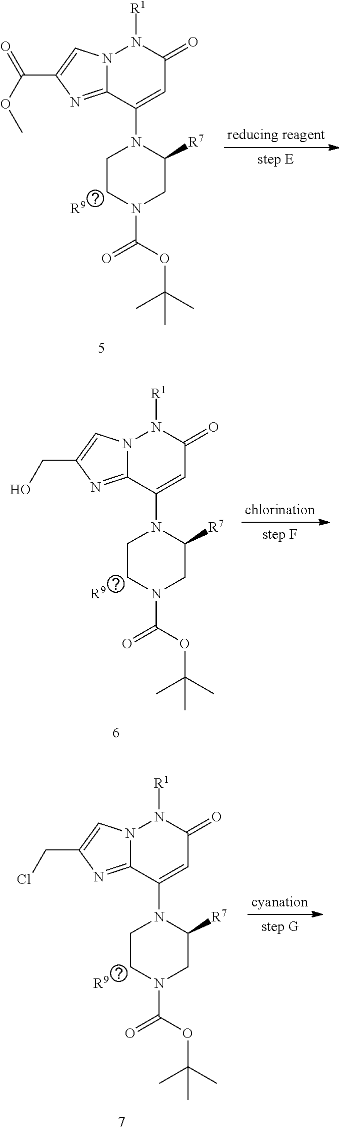

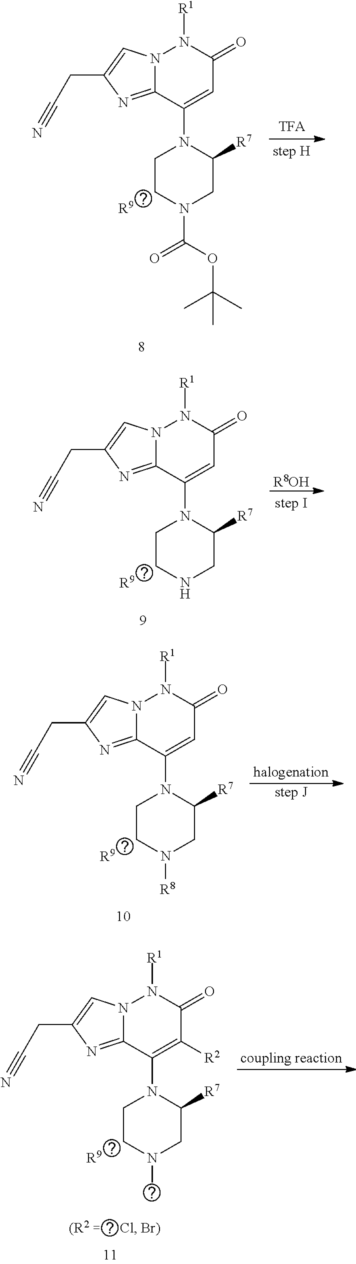

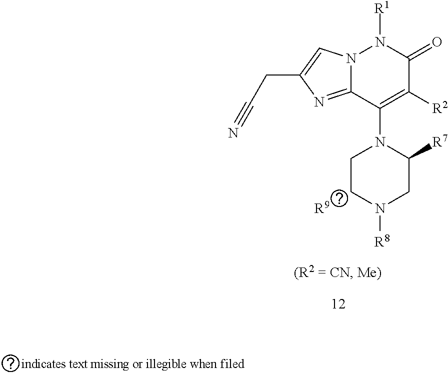

In scheme I, Compound 1 is reacted with ethyl 3-bromo-2-oxopropanoate under heating condition via cyclization reaction to give Compound 2. Compound 2 is reacted with the appropriate chiral secondary amine by nucleophilic aromatic substitution reaction to give Compound 3 which is used to give a Compound 4 by Pd-catalyzed C—O coupling reaction with appropriate Pd catalyst (such as Pd₂(dba)₃) and ligand (such as tBuXPhos) under basic condition (such as KOH). Compound 4 is reacted with appropriate R¹—X under basic condition (such as K₂CO₃, Cs₂CO₃) to give Compound 5. Reduction of Compound 5 gives Compound 6 as an alcohol using a reducing agent (such as NaBH₄). The hydroxyl group of Compound 6 is chlorinated by treatment with chlorination agent (such as SOCl₂) to give Compound 7. Compound 7 is converted into corresponding Compound 8 by treatment with cyanation agent (such as TMSCN) under basic condition (such as Cs₂CO₃ or nBu₄NF). Compound 8 is deprotected under acidic condition (such as TFA or 4M solution of HCl in 1,4-dioxane) to give Compound 9. Tertiary amines Compound 10 is prepared by N-alkylation of secondary amines Compound 9 via treatment with reductive alkylation with aldehydes or ketones or a phosphonium salt mediated alkylation of amines with corresponding alcohols (Florencio Zaragoza and Henrik Stephensen, J. Org. Chem. 2001, 66, 2518-2521). The halogenation of the Compound 10 with electrophilic halogenating reagents (such as NBS, NCS, or selectfluor) gives Compound 11 (wherein R² is F, Cl, or Br). The Compound 11 (wherein R² is Br) also can be used to produce a Compound 12 (wherein R² is CN, or Me) by Pd-catalyst coupling reaction with Zn(CN)₂ and 2,4,6-Trimethylboroxin.

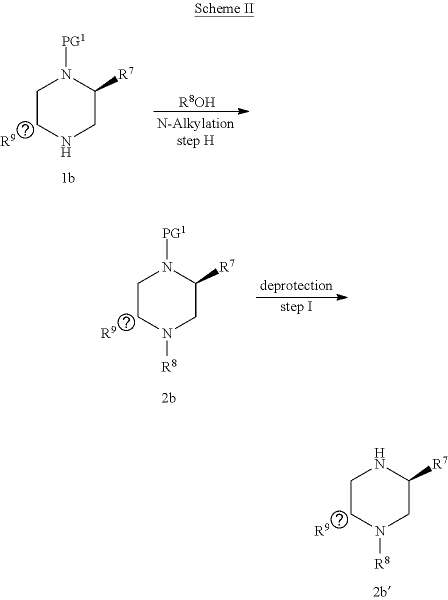

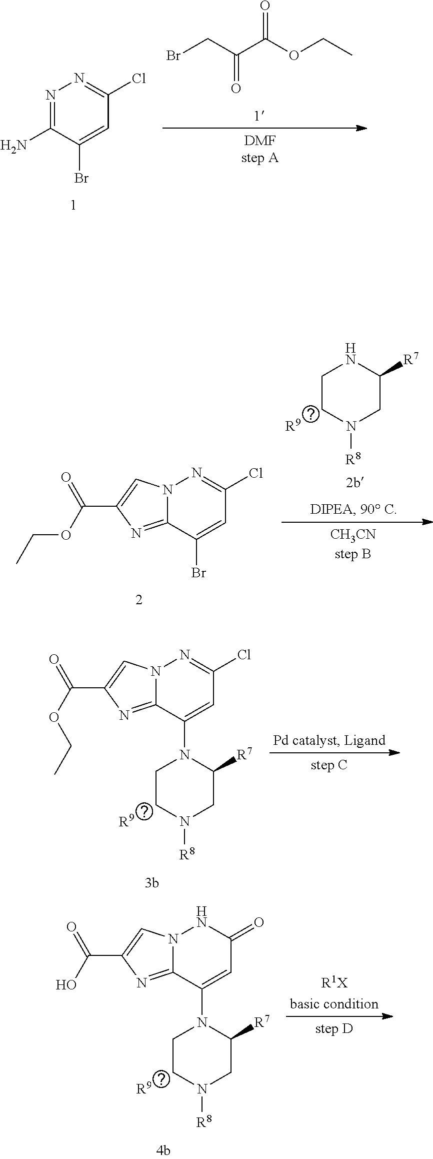

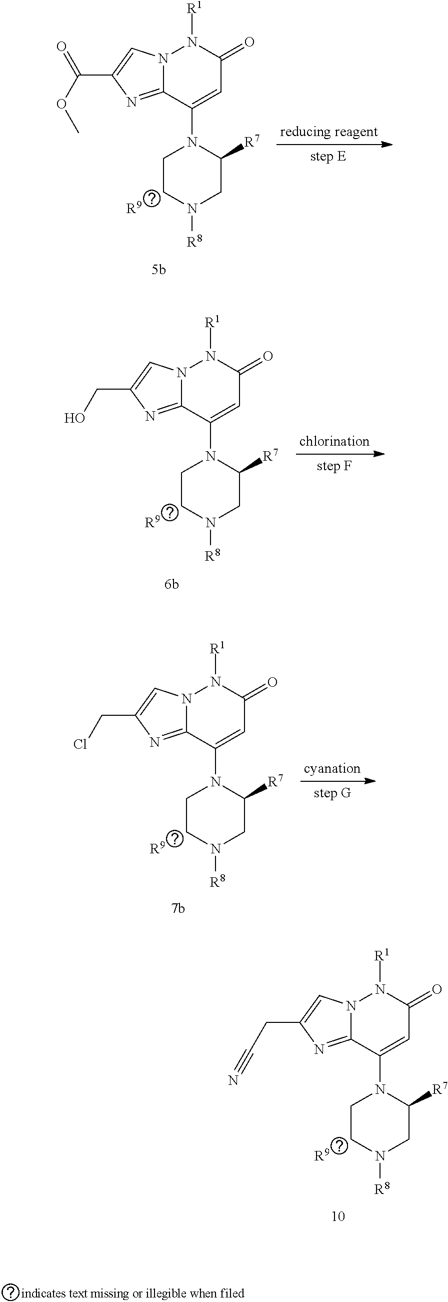

Scheme II is an alternative route for target compounds with similar reactions. In scheme II, Tertiary amines Compound 2b is prepared by N-alkylation of a secondary amines and commercially available Compound 1b by treatment with reductive alkylation with aldehydes or ketones, or a phosphonium salt mediated alkylation of amines with corresponding alcohols (Florencio Zaragoza and Henrik Stephensen, J. Org. Chem. 2001, 66, 2518-2521). Compound 2b is deprotected using acid condition (such as TFA or 4M solution of HCl in 1,4-dioxane) to give Compound 2b′. Compound 2 is reacted with Compound 2b′ by nucleophilic aromatic substitution reaction to give Compound 3b which is used to give a Compound 4b by Pd-catalyzed C—O coupling reaction with appropriate Pd catalyst (such as Pd₂(dba)₃) and ligand (such as tBuXPhos) under basic condition (such as KOH). Compound 4b is reacted with appropriate R1-X under basic condition (such as K₂CO₃, Cs₂CO₃) to give Compound 5b. Reduction of Compound 5b gives Compound 6b as an alcohol using a reducing agent (such as NaBH₄). The hydroxyl group of Compound 6b is chlorinated by treatment with chlorination agent (such as SOCl₂) to give Compound 7b. Compound 7b is converted into corresponding Compound 10 by treatment with cyanation agent (such as TMSCN) under basic condition (such as Cs₂CO₃ or nBu₄NF).

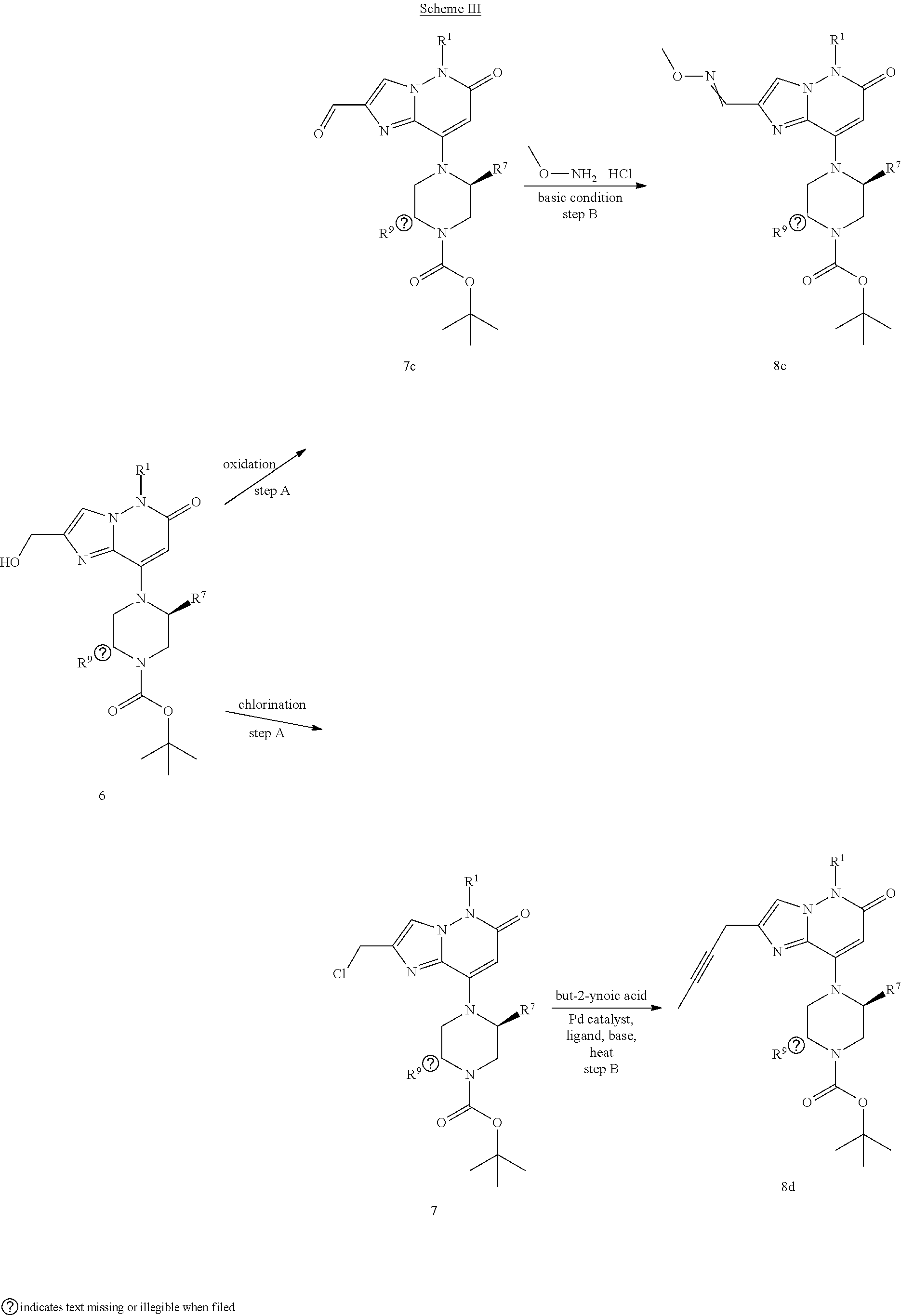

In scheme III, Compound 7c is prepared by oxidation reaction of Compound 6 using oxidant (such as MnO₂, Dess-Martin reagent). Compound 7c is reacted with O-methylhydroxylamine hydrochloride by condensation reaction to give Compound 8c using basic condition (such as CH₃COONa, K₂CO₃). Compound 8c is used to be converted into corresponding target compounds with similar reactions to scheme I. Compound 8d is prepared by alkynylation of Compound 7 using Pd-catalyzed C—C coupling reaction with appropriate Pd catalyst (such as Pd(OAc)₂) and ligand (such as X-Phos) under basic condition (such as Cs₂CO₃). Compound 8d is used to be converted into corresponding target compounds with similar reactions to scheme I.

### EXAMPLES

The examples below are intended to be purely exemplary and should not be considered to be limiting in any wav. Unless otherwise specified, the experimental methods in the Examples described below are conventional methods. Unless otherwise specified, the reagents and materials are all commercially available. All solvents and chemicals employed are of analytical grade or chemical purity. Solvents are all redistilled before use. Anhydrous solvents are all prepared according to standard methods or reference methods. Silica gel (100-200 meshes) for flash column chromatography and silica gel (GF254) for thin-layer flash column chromatography (TLC) are commercially available from Tsingdao Haiyang Chemical Co., Ltd. or Yantai Chemical Co., Ltd. of China; all are eluted with petroleum ether (60-90° C.)/ethyl acetate (v/v), and visualized by iodine or the solution of molybdphosphoric acid in ethanol unless otherwise specified. All extraction solvents, unless otherwise specified, are dried over anhydrous Na₂SO₄. ¹H NMR spectra are recorded on Bruck-400 nuclear magnetic resonance spectrometer with TNMS (tetramethylsilane) as the internal standard. LC/MS data are recorded by using Agilent1100 High Performance Liquid Flash column chromatography-Ion Trap Mass Spectrometer (LC-MSD Trap) equipped with a diode array detector (DAD) detected at 214 nm and 254 nm, and an ion trap (ESI source). All compound names except the reagents were generated by ChemDraw®.

### Synthesis

Preparative HPLC Conditions (Method A)

| | |
|---|---|
| Column | Phenomenex Gemini NX-C18, 150 × |
| | 21.2 mm, 5 μm |
| Column Temp. | R.T. |
| Detection Wavelength | DAD, UV λ = 214/254 nm |
| Run Time | 17.0 min |
| Flow Rate | 20.0 mL/min |
| Mobile Phase A | 0.1% FA-H₂O (v/v) |
| Mobile Phase B | 0.1% FA-CH₃CN (v/v) |

Preparative HPLC (Method B)

| | |
|---|---|
| Column | Waters XSelect CSH C18, 150 × 19 mm, 5 μm |
| Column Temp. | R.T. |
| Detection Wavelength | DAD, UV λ = 214/254 nm |
| Run Time | 17.0 min |
| Flow Rate | 17 mL/min |
| Mobile Phase A | 0.03% NH₃ H₂O—H₂O (v/v) |
| Mobile Phase B | CH³CN |

| | |
|---|---|
| Ac | Acetyl |
| AcOH | Acetic acid |
| Aq | Aqueous |
| Brine | Saturated aqueous sodium chloride solution |
| Bn | Benzyl |
| Boc | Tert-butyloxycarbonyl |
| Cbz | Benzyloxycarbonyl |
| CDI | N,N′-Carbonyldiimidazole |
| DMF | N,N-Dimethylformamide |
| Dppf | 1,1″-bis(diphenylphosphino)ferrocene |
| DBU | 1,8-diazabicyclo[5.4.0]undec-7-ene |
| DCE | 1,2-dichloroethane |
| DCM | Dichloromethane |
| DIPEA | N,N-diisopropylethylamine |
| DMF | N,N-dimethylformamide |
| DMSO | Dimethyl sulfoxide |
| EA | Ethyl acetate |
| eq | Equivalent |
| g | Grams |
| HATU | O-(7-Azabenzotriazol-1-yl)-N,N,N′,N′-tetramethyluronium |
| | hexafluorophosphate |
| HCl | Hydrochloric acid |
| HPLC | High-performance liquid flash column chromatography |
| HMDS | bis(trimethylsilyl)amine |
| IPA | Isopropyl alcohol |
| i-PrOH | Isopropyl alcohol |
| mg | Milligrams |
| mL | Milliliters |
| mmol | Millimole |
| MeCN | Acetonitrile |
| MeOH | Methanol |
| Min | Minutes |
| MS | Mass spectrum |
| m-CPBA | 3-Chloroperoxybenzoic acid |
| NMR | Nuclear magnetic resonance |
| NCS | N-Chlorosuccinimide |
| NBS | N-Bromosuccinimide |
| NFSI | N-fluorobisbenzenesulfonamide |
| OTf | Trifluoromethanesulfonate |
| Pd/C | Palladium on carbon |
| Pd₂(dba)₃ | Tris(dibenzylideneacetone)dipalladium |
| PE | Petroleum ether |
| RT | room temperature |
| SEM | 2-trimethylsilylethoxymethoxy(2-(chloromethoxy)eth- |
| | yl)trimethylsilane |
| TBAF | Tetrabutylammonium fluoride |
| TBSCl | tert-Butyldimethylsilyl chloride |
| TMSCN | Trimethylsilyl cyanide |
| TMSOTf | trimethylsilyl trifluoromethanesulfonate |
| tBuXPhos | Bis(1,1-dimethylethyl)[2′,4′,6′-tris(1- |
| | methylethyl)[1,1′-biphenyl]-2-yl]phosphine |
| tBuXPhos | Methanesulfonato(2-di-t-butylphosphino-2′,4′,6′-tri-i-propyl- |
| Pd G3 | 1,1′-biphenyl)(2′-amino-1,1′-biphenyl-2-yl)palladium(II) |
| TEA | Triethanolamine |
| TFA | Trifluoroacetic acid |
| THF | Tetrahydrofuran |
| THP | tetrahydropyran3,4-dihydro-2H-pyran |
| TLC | thin layer flash column chromatography |
| X-Phos | dicyclohexyl(2′,4′,6′-triisopropyl-[1,1′-biphenyl]-2- |
| | yl)phosphane |

### Intermediate 1: 2-(8-((2S,5R)-2,5-dimethylpiperazin-1-yl)-5-methyl-6-oxo-5,6-dihydroimidazo[1,2-b]pyridazin-2-yl)acetonitrile

### Step A: ethyl 8-bromo-6-chloroimidazo[1,2-b]pyridazine-2-carboxylate

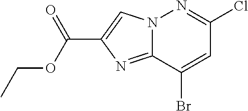 [SMILES: CCOC(=O)c1cn2nc(Cl)cc(Br)c2n1]

To a stirred solution of 4-bromo-6-chloropyridazin-3-amine (4.16 g, 20 mmol) in DMF (40 mL) was added ethyl 2-bromo-2-oxoacetate (7.8 g, 40 mmol). After the addition, the reaction mixture was stirred overnight under N₂. The reaction mixture was poured into ice-water (150 mL), stirred for 30 mins and filtered. The fliltered cake was collected, dried to give the titledt compound (4.5 g, 74%). MS: M/e 304/305 (M+1)⁺.

### Step B: ethyl 8-((2S,5R)-4-(tert-butoxycarbonyl)-2,5-dimethylpiperazin-1-yl)-6-chloroimidazo[1,2-b]pyridazine-2-carboxylate

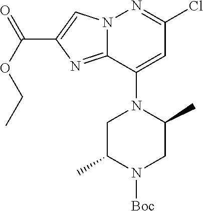 [SMILES: CCOC(=O)c1cn2nc(Cl)cc(N3C[C@@H](C)N(C(=O)OC(C)(C)C)C[C@@H]3C)c2n1]

A mixture of ethyl 8-bromo-6-chloroimidazo[1,2-b]pyridazine-2-carboxylate (3.04 g, 10 mmol), tert-butyl (2R,5S)-2,5-dimethylpiperazine-1-carboxylate (2.57 g, 12 mmol) and DIPEA (2.58 g, 20 mmol) in CH₃CN (40 mL) was stirred at 80° C. overnight. The reaction mixture was poured into H₂O (50 mL), extracted with EtOAc (30 mL×3). The combined organic layers were washed with brine, dried over Na₂SO₄, concentrated. The resulting residue was further purified by flash column chromatography to give the titled compound (3.87 g, 89%). MS: M/e 438 (M+1)⁺.

### Step C: 8-((2S,5R)-4-(tert-butoxycarbonyl)-2,5-dimethylpiperazin-1-yl)-6-oxo-5,6-dihydroimidazo[1,2-b]pyridazine-2-carboxylic acid

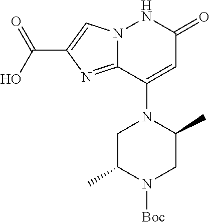 [SMILES: C[C@@H]1CN(c2cc(=O)[nH]n3cc(C(=O)O)nc23)[C@@H](C)CN1C(=O)OC(C)(C)C]

To a stirred solution of ethyl 8-((2S,5R)-4-(tert-butoxycarbonyl)-2,5-dimethylpiperazin-1-yl)-6-chloroimidazo[1,2-b]pyridazine-2-carboxylate (41.4 mg, 0.1 mmol) in 1,4-dioxane (40 mL) was added aq.KOH (1.48 g, 26.55 mmol, in 10 mL H₂O), then Pd₂(dba)₃ (0.4 g, 0.44 mmol) and t-BuXPhos (0.376 g, 0.885 mmol) were added under N2. After the addition, the reaction mixture was stirred at 90° C. for 3 hours. The reaction mixture was poured into H₂O (100 mL) and extracted with EtOAc (50 mL×2). The combined organic layers were discarded and the aqueous layer was acidified to pH=3-4 with aq.citric acid, and extracted with EtOAc (100 mL×3). The combined organic layers were washed with brine, dried over Na₂SO₄, concentrated to give the target compound (4.45 g, crude, 100%). MS: M/e 392 (M+1)+.

### Step D: methyl 8-((2S,5R)-4-(tert-butoxycarbonyl)-2,5-dimethylpiperazin-1-yl)-5-methyl-6-oxo-5,6-dihydroimidazo[1,2-b]pyridazine-2-carboxylate

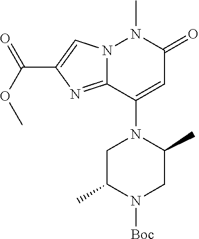 [SMILES: COC(=O)c1cn2c(n1)c(N1C[C@@H](C)N(C(=O)OC(C)(C)C)C[C@@H]1C)cc(=O)n2C]

A mixture of 8-((2S,5R)-4-(tert-butoxycarbonyl)-2,5-dimethylpiperazin-1-yl)-6-oxo-5,6-dihydroimidazo[1,2-b]pyridazine-2-carboxylic acid (8.85 mmol), Mel (3.77 g, 26.55 mmol) and Cs₂CO₃ (8.6 g, 26.55 mmol) in 1,4-dioxane (50 mL) was stirred at 90° C. for 2 days in a sealed tube. The reaction mixture was poured into H₂O (50 mL) and extracted with EtOAc (50 mL×3). The combined organic layers were washed with brine, dried over Na₂SO₄, concentrated. The resulting residue was purified by flash column chromatography to give the titled compound (2.2 g, 59%). ¹H NMR (400 MHz, CDCl₃) δ 7.96 (s, 1H), 5.88 (s, 1H), 4.91 (s, 1H), 4.53 (s, 1H), 4.35 (s, 1H), 3.95 (s, 3H), 3.86-3.73 (m, 4H), 3.66-3.41 (m, 2H), 1.49 (s, 9H), 1.31-1.19 (m, 6H) ppm. MS: M/e 420 (M+1)⁺.

### Step E: tert-butyl (2R,5S)-4-(2-(hydroxymethyl)-5-methyl-6-oxo-5,6-dihydroimidazo[1,2-b]pyridazin-8-yl)-2,5-dimethylpiperazine-1-carboxylate

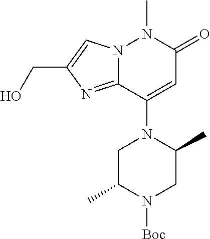 [SMILES: C[C@@H]1CN(c2cc(=O)n(C)n3cc(CO)nc23)[C@@H](C)CN1C(=O)OC(C)(C)C]

To a stirred solution of methyl 8-((2S,5R)-4-(tert-butoxycarbonyl)-2,5-dimethylpiperazin-1-yl)-5-methyl-6-oxo-5,6-dihydroimidazo[1,2-b]pyridazine-2-carboxylate (1.1 g, 2.63 mmol) in EtOH (20 mL) was added NaBH₄ (0.3 g, 7.88 mmol). After the addition, the reaction was stirred at 70° C. for 2 days. The reaction mixture was diluted with H₂O (20 mL), extracted with EtOAc (20 mL×4). The combined organic layers were washed with brine, dried over Na₂SO₄ and concentrated to give the titled compound (500 mg, 49%). MS: M/e 392 (M+1)⁺.

### Step F: tert-butyl (2R,5S)-4-(2-(chloromethyl)-5-methyl-6-oxo-5,6-dihydroimidazo[1,2-b]pyridazin-8-yl)-2,5-dimethylpiperazine-1-carboxylate

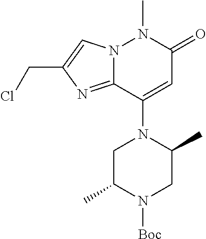 [SMILES: C[C@@H]1CN(c2cc(=O)n(C)n3cc(CCl)nc23)[C@@H](C)CN1C(=O)OC(C)(C)C]

To a stirred solution of tert-butyl (2R,5S)-4-(2-(hydroxymethyl)-5-methyl-6-oxo-5,6-dihydroimidazo[1,2-b]pyridazin-8-yl)-2,5-dimethylpiperazine-1-carboxylate (200 mg, 0.512 mmol) in CH₂Cl₂ (10 mL) was added SOCl₂ (122 mg, 1.02 mmol). After the addition, the reaction was stirred for an hour. The reaction mixture was washed with aq.NaHCO₃, brine, dried over Na₂SO₄ and concentrated to give the titled compound (170 mg, 81%). MS: M/e 410 (M+1)⁺.

### Step G: tert-butyl (2R,5S)-4-(2-(cyanomethyl)-5-methyl-6-oxo-5,6-dihydroimidazo[1,2-b]pyridazin-8-yl)-2,5-dimethylpiperazine-1-carboxylate

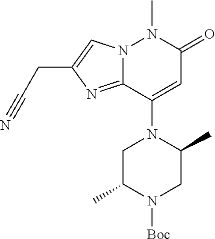 [SMILES: C[C@@H]1CN(c2cc(=O)n(C)n3cc(CC#N)nc23)[C@@H](C)CN1C(=O)OC(C)(C)C]

A mixture of tert-butyl (2R,5S)-4-(2-(chloromethyl)-5-methyl-6-oxo-5,6-dihydroimidazo[1,2-b]pyridazin-8-yl)-2,5-dimethylpiperazine-1-carboxylate (270 mg, 0.66 mmol), TMSCN (130 mg, 1.32 mmol) and Cs₂CO₃ (645 mg, 1.98 mmol) in CH₃CN (5 mL) was stirred at 70° C. overnight. The reaction mixture was diluted with EtOAc (15 mL), washed with H₂O, brine, dried over Na₂SO₄, concentrated and purified by flash column chromatography to give the titled compound (100 mg, 38%). MS: M/e 401 (M+1)⁺.

### Step H: 2-(8-((2S,5R)-2,5-dimethylpiperazin-1-yl)-5-methyl-6-oxo-5,6-dihydroimidazo[1,2-b]pyridazin-2-yl)acetonitrile

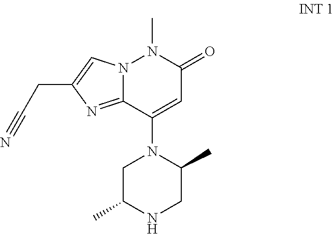 [SMILES: C[C@@H]1CN(c2cc(=O)n(C)n3cc(CC#N)nc23)[C@@H](C)CN1]

To a stirred solution of tert-butyl (2R,5S)-4-(2-(cyanomethyl)-5-methyl-6-oxo-5,6-dihydroimidazo[1,2-b]pyridazin-8-yl)-2,5-dimethylpiperazine-1-carboxylate (100 mg, 0.24 mmol) in CH₂Cl₂ (5 mL) was added TFA (1 mL). After the addition, the reaction was stirred for 3 hours. The reaction mixture was concentrated to give the residue, which was basified to pH=10˜12 with aq.Na₂CO₃, extracted with CH₂Cl₂ (15 mL×6). The combined organic layers were washed with brine, dried over Na₂SO₄, concentrated to give the target compound (70 mg, 94%). MS: M/e 301 (M+1)⁺

### Intermediate 2: (2R,5S)-1-benzyl-2,5-diethylpiperazine

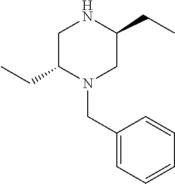 [SMILES: CC[C@H]1CN(Cc2ccccc2)[C@H](CC)CN1]

### Step A: methyl (R)-2-(benzylamino)butanoate

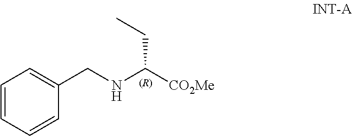

To a solution of methyl (R)-2-aminobutanoate (100.0 g, 0.85 mol) in CH₃CN (1000 mL) was added benzyl bromide (146.1 g, 0.85 mol) at 0° C. under N₂ atmosphere. The reaction was stirred at room temperature overnight and concentrated under reduced pressure. The resulting residue was dissolved in EA (1000 mL) and washed with water (1000 mL×3). The organic layers were concentrated under reduced pressure. The resulting residue was purified by flash column chromatography (PE:EA=10:1) to give the titled compound (106 g, 60%). MS: M/e 208 (M+1)⁺.

### Step B: methyl (R)-2-((S)—N-benzyl-2-((tert-butoxycarbonyl)amino)butanamido)butanoate

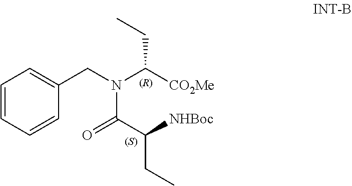

To a solution of methyl (R)-2-(benzylamino)butanoate (117.0 g, 0.56 mol), (S)-2-((tert-butoxycarbonyl)amino)butanoic acid (170.5 g, 0.84 mmol) and 4-Methylmorpholine (113.1 g, 1.12 mmol) in DCM (2000 mL) was added HATU (319.0 g, 0.84 mmol) at 0° C. The reaction was stirred at room temperature overnight, and quenched by water and washed with water (1500 mL×2). The organic layers were concentrated under reduced pressure. The resulting residue was purified by flash column chromatography (PE:EA=10:1) to give the titled compound product (178 g, 80%). MS: M/e 393 (M+1)⁺.

### Step C: methyl (R)-2-((S)-2-amino-N-benzylbutanamido)butanoate

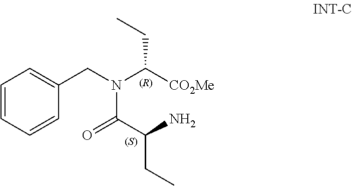

To a solution of methyl (R)-2-((S)—N-benzyl-2-((tert-butoxycarbonyl)amino)butanamido)butanoate (178 g, 0.45 mol) in 1,4-dioxane (100 mL) was added HCl (400 mL, 4 M in 1,4-dioxane) at room temperature. The resulting mixture was stirred at room temperature for another 2 hours, and concentrated under vacuum to give the crude product (200 g, crude). MS: M/e 293(M+1)⁺.

### Step D: (3S,6R)-1-benzyl-3,6-diethylpiperazine-2,5-dione

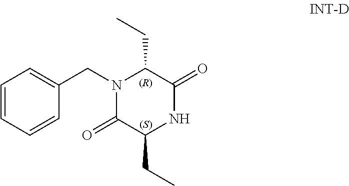

To a solution of methyl (R)-2-((S)-2-amino-N-benzylbutanamido)butanoate (200 g, crude) in EA (1000 mL) was added aq. NaHCO₃ (300 mL) at room temperature. The reaction mixture was stirred at room temperature for another 2 hours. The organic layers were concentrated under reduced pressure. The resulting residue was triturated with MTBE to give the titled compound (61 g, 52% for 2 steps, ee: 97%). MS: M/e 261 (M+1)⁺.

### Step E: (2R,5S)-1-benzyl-2,5-diethylpiperazine

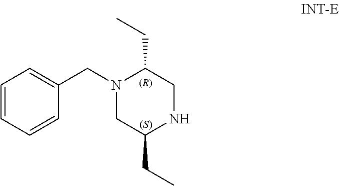

To a solution of LiAlH4 (26.5 g, 0.69 mol) in THF (1000 mL) was added slowly (3S,6R)-1-benzyl-3,6-diethylpiperazine-2,5-dione (61.0 g, 0.23 mol) in THF (500 mL) at 0° C. The resulting mixture was stirred at room temperature for 2 hours, then stirred at 80° C. overnight. The reaction was quenched by water (27 mL) slowly at 0° C. Then, 1N aqueous NaOH solution (54 mL) and water (81 mL) was added sequentially. The resulting mixture was stirred for 2 hours. The white precipitates that formed was removed by filtration. The filter cake was washed with EA (500 mL). The combined filtrates were evaporated. The resulting residue was dissolved in toluene. The solvent was removed under vacuum to dryness to afford the titled compound (51 g, 95%). MS: M/e 233 (M+1)⁺.

### Compound A1: 2-(8-((2S,5R)-2,5-dimethyl-4-(1-(quinoxalin-6-yl)ethyl)piperazin-1-yl)-5-methyl-6-oxo-5,6-dihydroimidazo[1,2-b]pyridazin-2-yl)acetonitrile

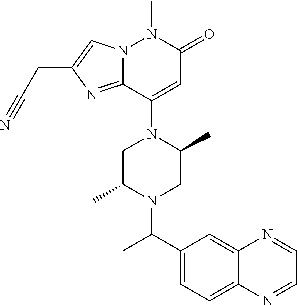 [SMILES: CC(c1ccc2nccnc2c1)N1C[C@H](C)N(c2cc(=O)n(C)n3cc(CC#N)nc23)C[C@H]1C]

A mixture of 2-(8-((2S,5R)-2,5-dimethylpiperazin-1-yl)-5-methyl-6-oxo-5,6-dihydroimidazo[1,2-b]pyridazin-2-yl)acetonitrile (80 mg, 0.27 mmol), 1-(quinoxalin-6-yl)ethan-1-ol (139 mg, 0.8 mmol), (cyanomethyl)trimethylphosphonium iodide (194 mg, 0.8 mmol) and DIPEA (348 mg, 2.7 mmol) in CH₃CN (4 mL) was stirred at 100° C. for 6 hours in a sealed tube. The reaction mixture was diluted with EtOAc (10 mL), washed with brine, dried over Na₂SO₄, concentrated. The resulting residue was purified by Pre-TLC (CH₂Cl₂:MeOH=10:1) to give the titled Compound A1 (47 mg). The another batch Compound A1(47 mg) was separated into Compound Ala (16 mg) and Compound Alb (17 mg) by chiral Prep-HPLC. The chiral separation conditions r shown below.

| | |
|---|---|
| Column | CHIRALPAK IH |
| Column Size | 2 cm × 25 cm, 5 um |
| Mobile Phase A | MtBE(20 mM NH3—MEOH) |
| Mobile Phase B | MeOH |
| Flow Rate | 20 mL/min |
| Wave Length | UV 220 nm |
| Temperature | 25° C. |
| Prep-HPLC Equipment | Prep-HPLC-Gilson |

Compound A1: ¹H NMR (400 MHz, DMSO-d6) δ 8.93 (s, 2H), 8.15-7.92 (m, 3H), 7.87 (s, 1H), 5.52 (s, 1H), 5.29-4.55 (m, 1H), 4.04 (s, 2H), 3.97-3.75 (m, 1H), 3.61 (s, 3H), 3.58-3.50 (m, 1H), 3.28 (s, 1H), 3.01-2.65 (m, 2.5H), 2.10 (d, J=12.0 Hz, 0.5H), 1.42-1.31 (m, 4.5H), 1.12 (s, 3H), 0.97 (d, J=5.8 Hz, 1.5H) ppm. MS: M/e 457 (M+1)⁺.

Compound A1a (the earlier peak): ¹H NMR (400 MHz, DMSO-d6) δ 8.96-8.91 (m, 2H), 8.17-7.94 (m, 3H), 7.87 (s, 1H), 5.52 (s, 1H), 5.21 (s, 1H), 4.04 (s, 2H), 3.98-3.89 (m, 1H), 3.61 (s, 3H), 3.32-3.23 (m, 1.5H), 3.02-2.74 (m, 3.5H), 1.41-1.29 (m, 6H), 1.00-0.94 (m, 3H) ppm. MS: M/e 457 (M+1)⁺.

Compound A1b (the later peak): ¹H NMR (400 MHz, DMSO-d6) δ 8.96-8.90 (m, 1H), 8.17-7.92 (m, 3H), 7.87 (s, 1H), 5.53 (s, 1H), 4.67 (s, 1H), 4.04 (s, 2H), 3.84-3.76 (m, 1H), 3.68-3.49 (m, 5H), 2.78-2.70 (m, 1H), 2.14-2.07 (m, 1H), 1.42-1.35 (m, 3H), 1.18-1.08 (m, 6H) ppm. MS: M/e 457 (M+1)⁺.

Compound A2: 2-(8-((2S,5R)-2,5-dimethyl-4-(1-(3-methylquinoxalin-6-yl)ethyl)piperazin-1-yl)-5-methyl-6-oxo-5,6-dihydroimidazo[1,2-b]pyridazin-2-yl)acetonitrile

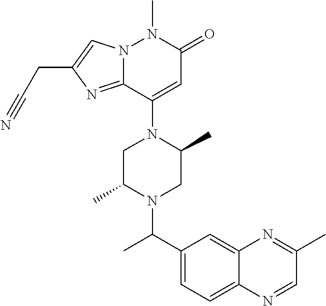 [SMILES: Cc1cnc2ccc(C(C)N3C[C@H](C)N(c4cc(=O)n(C)n5cc(CC#N)nc45)C[C@H]3C)cc2n1]

### Step A: 2-(8-((2S,5R)-2,5-dimethylpiperazin-1-yl)-5-methyl-6-oxo-5,6-dihydroimidazo[1,2-b]pyridazin-2-yl)acetonitrile

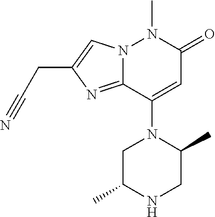 [SMILES: C[C@@H]1CN(c2cc(=O)n(C)n3cc(CC#N)nc23)[C@@H](C)CN1]

A solution of tert-butyl (2R,5S)-4-(2-(cyanomethyl)-5-methyl-6-oxo-5,6-dihydroimidazo[1,2-b]pyridazin-8-yl)-2,5-dimethylpiperazine-1-carboxylate (570 mg, 1.43 mmol) and TFA (2 ml) in DCM (10 ml) was stirred at rt for 1 h. The reaction was poured into aq. NaHCO₃ (sat., 10 ml) and then extracted with organic solvent (DCM:MeOH=10:1). The organic layers were dried over Na₂SO₄ and concentrated to dryness to give the titled compound (420 mg, 98%), which was used directly for the next step without further purification. MS: M/e 301 (M+1)⁺.

### Step B: 2-(8-((2S,5R)-2,5-dimethyl-4-(1-(3-methylquinoxalin-6-yl)ethyl)piperazin-1-yl)-5-methyl-6-oxo-5,6-dihydroimidazo[1,2-b]pyridazin-2-yl)acetonitrile

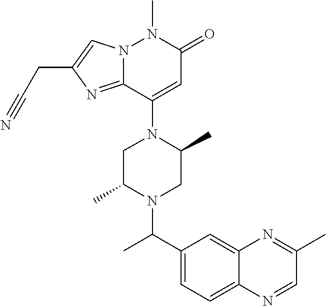 [SMILES: Cc1cnc2ccc(C(C)N3C[C@H](C)N(c4cc(=O)n(C)n5cc(CC#N)nc45)C[C@H]3C)cc2n1]

A solution of 2-(8-((2S,5R)-2,5-dimethylpiperazin-1-yl)-5-methyl-6-oxo-5,6-dihydroimidazo[1,2-b]pyridazin-2-yl)acetonitrile (210 mg, 0.70 mmol), 1-(3-methylquinoxalin-6-yl)ethan-1-ol (165 mg, 0.88 mmol), (cyanomethyl)trimethylphosphonium iodide (510 mg, 2.10 mmol) and DIPEA (903 mg, 7 mmol) in MeCN (4 ml) was stirred at 100° C. for 2 days. The reaction was diluted with EA (15 ml) and washed with brine (10 ml). The organic layer was concentrated to dryness. The resulting residue was purified by flash column chromatography to give the titled Compound A2 (290 mg), which was separated into Compound A2a (106 mg) and Compound A2b (87 mg) by chiral Prep-HPLC. The chiral separation conditions are shown below.

| | |
|---|---|
| Column | CHIRAL Cellulose-SB |
| Column Size | 2 cm × 25 cm, 5 um |
| Mobile Phase A | MtBE |
| Mobile Phase B | MeOH:DCM |
| Flow Rate | 20 mL/min |
| Wave Length | UV 220 nm |
| Temperature | 25° C. |
| Prep-HPLC Equipment | Prep-HPLC-Gilson |

Compound A2a (the earlier peak):¹H NMR (400 MHz, DMSO-d6) δ 8.81 (s, 1H), 8.02 (d, J=8.6 Hz, 1H), 7.92 (s, 1H), 7.88-7.81 (m, 2H), 5.53 (s, 1H), 4.67 (s, 1H), 4.03 (s, 2H), 3.76 (q, J=6.6 Hz, 1H), 3.61 (s, 3H), 3.54 (d, J=12.5 Hz, 2H), 3.31-3.27 (m, 1H), 2.75-2.70 (m, 1H), 2.70 (s, 3H), 2.11 (d, J=11.6 Hz, 1H), 1.37 (d, J=6.5 Hz, 3H), 1.12 (dd, J=6.2, 3.8 Hz, 6H) ppm. MS: M/e 471 (M+1)⁺.

Compound A2b (the later peak):¹H NMR (400 MHz, DMSO-d6) δ 8.82 (s, 1H), 8.05 (d, J=8.6 Hz, 1H), 7.95 (d, J=1.5 Hz, 1H), 7.89-7.84 (m, 2H), 5.52 (s, 1H), 5.20 (s, 1H), 4.03 (s, 2H), 3.89 (q, J=6.5 Hz, 1H), 3.61 (s, 3H), 3.31-3.26 (m, 2H), 2.95 (dd, J=11.6, 3.7 Hz, 1H), 2.87-2.76 (m, 2H), 2.70 (s, 3H), 1.33 (dd, J=12.1, 6.5 Hz, 6H), 0.96 (d, J=6.5 Hz, 3H) ppm. MS: M/e 471 (M+1)⁺.

Compound A3: 8-((2S,5R)-2,5-dimethyl-4-(1-(quinoxalin-6-yl)ethyl)piperazin-1-yl)-2-(hydroxymethyl)-5-methylimidazo[1,2-b]pyridazin-6(5H)-one

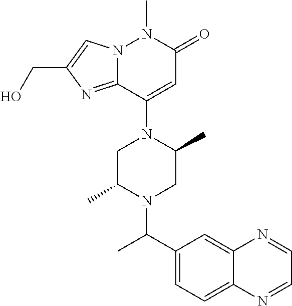 [SMILES: CC(c1ccc2nccnc2c1)N1C[C@H](C)N(c2cc(=O)n(C)n3cc(CO)nc23)C[C@H]1C]

### Step A: 6-(1-bromoethyl)quinoxaline

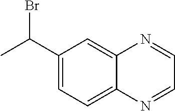

To a stirred solution of the 1-(quinoxalin-6-yl)ethan-1-ol (200 mg, 1.15 mmol) in aq.HBr (5 mL) was stirred at 60° C. for 4 hours. The reaction mixture was basified to pH=8-9 with aq.NaHCO₃, extracted with EtOAc (20 mL×3). The combined organic layers were washed with brine, dried over Na₂SO₄ and concentrated. The resulting residue was purified by flash column chromatography to give the titled compound (100 mg, 37%), which was solidified after standing for 2 hours. MS: M/e 237/239 (M+1)⁺.

### Step B: 8-((2S,5R)-2,5-dimethylpiperazin-1-yl)-2-(hydroxymethyl)-5-methylimidazo[1,2-b]pyridazin-6(5H)-one

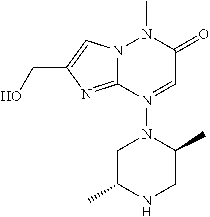

To a stirred solution of tert-butyl (2R,5S)-4-(2-(hydroxymethyl)-5-methyl-6-oxo-5,6-dihydroimidazo[1,2-b]pyridazin-8-yl)-2,5-dimethylpiperazine-1-carboxylate (39 mg, 0.1 mmol) in CH₂Cl₂ (5 mL) was added TFA (2 mL). Then the mixture was stirred for 2 hours. The reaction mixture was concentrated to give the titled compound, which was directly used to the next step. MS: M/e 292 (M+1)⁺.

### Step C: 8-((2S,5R)-2,5-dimethyl-4-(1-(quinoxalin-6-yl)ethyl)piperazin-1-yl)-2-(hydroxymethyl)-5-methylimidazo[1,2-b]pyridazin-6(5H)-one

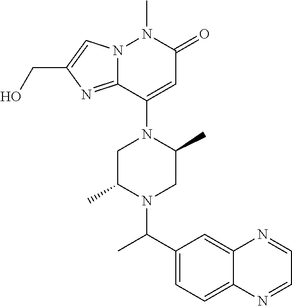 [SMILES: CC(c1ccc2nccnc2c1)N1C[C@H](C)N(c2cc(=O)n(C)n3cc(CO)nc23)C[C@H]1C]

A mixture of 8-((2S,5R)-2,5-dimethylpiperazin-1-yl)-2-(hydroxymethyl)-5-methylimidazo[1,2-b]pyridazin-6(5H)-one (crude, 0.1 mmol), DIPEA (39 mg, 0.3 mmol) and 6-(1-bromoethyl)quinoxaline (28 mg, 0.12 mmol) in CH₃CN (5 mL) was stirred at 70° C. overnight. The reaction mixture was poured into H₂O (15 mL) and extracted with EtOAc (10 mL×3). The combined organic layers were washed with brine, dried over Na₂SO₄, concentrated and purified by Pre-TLC (CH₂Cl₂:MeOH=10:1) to give the titled compound. ¹H NMR (400 MHz, DMSO-d6) δ 9.03-8.96 (m, 2H), 8.23-7.98 (m, 3H), 7.81 (s, 1H), 5.54 (d, J=9.6 Hz, 1H), 5.22 (s, 1H), 4.52 (s, 2H), 4.05-3.82 (m, 1H), 3.68 (s, 3H), 3.65-3.55 (m, 1H), 3.08-2.76 (m, 2.5H), 2.16 (d, J=12.8 Hz, 0.5H), 1.51-1.33 (m, 4.5H), 1.17 (s, 3H), 1.05-0.98 (m, 1.5H) ppm. MS: M/e 448 (M+1)⁺.

### Compound A4: 8-((2S,5R)-2,5-dimethyl-4-(1-(quinoxalin-6-yl)ethyl)piperazin-1-yl)-2-(methoxymethyl)-5-methylimidazo[1,2-b]pyridazin-6(5H)-one

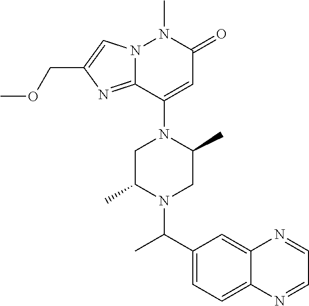 [SMILES: COCc1cn2c(n1)c(N1C[C@@H](C)N(C(C)c3ccc4nccnc4c3)C[C@@H]1C)cc(=O)n2C]

### Step A: tert-butyl (2R,5S)-4-(2-(methoxymethyl)-5-methyl-6-oxo-5,6-dihydroimidazo[1,2-b]pyridazin-8-yl)-2,5-dimethylpiperazine-1-carboxylate

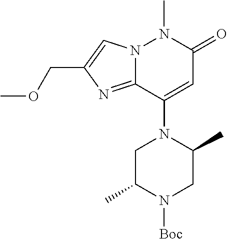

A mixture of tert-butyl (2R,5S)-4-(2-(chloromethyl)-5-methyl-6-oxo-5,6-dihydroimidazo[1,2-b]pyridazin-8-yl)-2,5-dimethylpiperazine-1-carboxylate (41 mg, 0.1 mmol) in MeOH (5 mL) was stirred at 50° C. overnight. The mixture was concentrated to give the titled compound, which was directly used to the next step. MS: M/e 406 (M+1)⁺.

### Step B: 8-((2S,5R)-2,5-dimethylpiperazin-1-yl)-2-(methoxymethyl)-5-methylimidazo[1,2-b]pyridazin-6(5H)-one

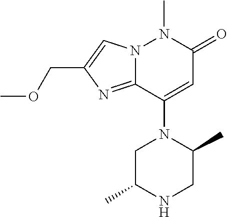

To a stirred solution of tert-butyl (2R,5S)-4-(2-(methoxymethyl)-5-methyl-6-oxo-5,6-dihydroimidazo[1,2-b]pyridazin-8-yl)-2,5-dimethylpiperazine-1-carboxylate (crude, 0.1 mmol) in CH₂Cl₂ (5 mL) was added TFA (2 mL). Then the mixture was stirred for an hour. The reaction mixture was concentrated to give the residue, which was washed with aq.K₂CO₃ and extracted with EtOAc (10 mL×3). The combined organic layers were washed with brine, dried over Na₂SO₄, concentrated to give the titled compound (30 mg, 100%). MS: M/e 306 (M+1)⁺.

### Step C: 8-((2S,5R)-2,5-dimethyl-4-(1-(quinoxalin-6-yl)ethyl)piperazin-1-yl)-2-(methoxymethyl)-5-methylimidazo[1,2-b]pyridazin-6(5H)-one

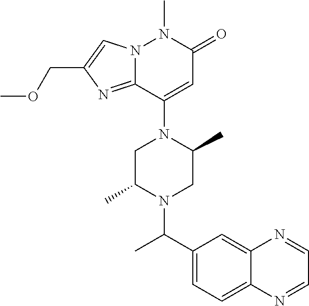 [SMILES: COCc1cn2c(n1)c(N1C[C@@H](C)N(C(C)c3ccc4nccnc4c3)C[C@@H]1C)cc(=O)n2C]

A mixture of 8-((2S,5R)-2,5-dimethylpiperazin-1-yl)-2-(methoxymethyl)-5-methylimidazo[1,2-b]pyridazin-6(5H)-one (30 mg, 0.1 mmol), 1-(quinoxalin-6-yl)ethan-1-ol (52.2 mg, 0.3 mmol), (cyanomethyl)trimethylphosphonium iodide (73 mg, 0.3 mmol) and DIPEA (90 mg, 0.7 mmol) in CH₃CN (3 mL) was stirred at 100° C. for 6 hours in a sealed tube. The reaction mixture was diluted with EtOAc (10 mL), washed with brine, dried over Na₂SO₄ and concentrated. The resulting residue was purified by Pre-TLC (CH₂Cl₂:MeOH=10:1) to give the titled compound (15 mg). ¹H NMR (400 MHz, DMSO-d6) δ 9.06-8.95 (m, 2H), 8.22-7.98 (m, 3H), 7.95 (s, 1H), 5.56 (d, J=6.8 Hz, 1H), 4.82-4.75 (m, 1H), 4.50-4.37 (m, 3H), 4.03-3.81 (m, 1H), 3.68 (s, 3H), 3.65-3.55 (m, 1H), 3.35 (s, 3H), 3.07-2.75 (m, 2H), 2.16 (d, J=12 Hz, 1H), 1.51-1.34 (m, 4.5H), 1.17 (s, 3H), 1.02 (d, J=5.6 Hz, 1.5H) ppm. MS: M/e 462 (M+1)⁺.

### Compound A5: 8-((2S,5R)-2,5-dimethyl-4-(1-(quinoxalin-6-yl)ethyl)piperazin-1-yl)-2-(1-methoxyethyl)-5-methylimidazo[1,2-b]pyridazin-6(5H)-one

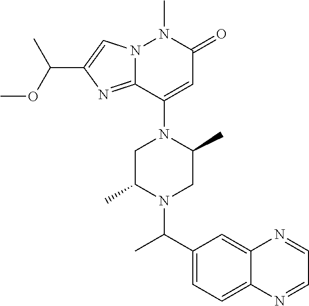 [SMILES: COC(C)c1cn2c(n1)c(N1C[C@@H](C)N(C(C)c3ccc4nccnc4c3)C[C@@H]1C)cc(=O)n2C]

### Step A: tert-butyl (2R,5S)-4-(2-formyl-5-methyl-6-oxo-5,6-dihydroimidazo[1,2-b]pyridazin-8-yl)-2,5-dimethylpiperazine-1-carboxylate

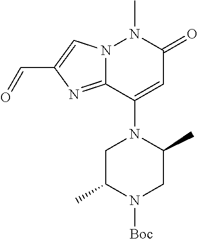 [SMILES: C[C@@H]1CN(c2cc(=O)n(C)n3cc(C=O)nc23)[C@@H](C)CN1C(=O)OC(C)(C)C]

To a stirred solution of tert-butyl (2R,5S)-4-(2-(hydroxymethyl)-5-methyl-6-oxo-5,6-dihydroimidazo[1,2-b]pyridazin-8-yl)-2,5-dimethylpiperazine-1-carboxylate in CH₂Cl₂ (10 mL) was added Dess-Martin periodinane (152 mg, 0.358 mmol) at room temperature. After the addition, the reaction mixture was stirred for 20 min. The reaction mixture was quenched with NaHCO₃ aq., extracted with CH₂Cl₂ (10 mL×2). The combined organic layers were washed with brine, dried over Na₂SO₄ and concentrated to give the titled compound which was directly used to the next step without further purification. MS: M/e 390 (M+1)⁺.

### Step B: tert-butyl (2R,5S)-4-(2-(1-hydroxyethyl)-5-methyl-6-oxo-5,6-dihydroimidazo[1,2-b]pyridazin-8-yl)-2,5-dimethylpiperazine-1-carboxylate

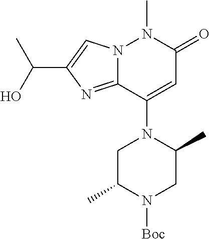 [SMILES: CC(O)c1cn2c(n1)c(N1C[C@@H](C)N(C(=O)OC(C)(C)C)C[C@@H]1C)cc(=O)n2C]

To a stirred solution of tert-butyl (2R,5S)-4-(2-formyl-5-methyl-6-oxo-5,6-dihydroimidazo[1,2-b]pyridazin-8-yl)-2,5-dimethylpiperazine-1-carboxylate (crude, 0.179 mmol) in dry THF (5 mL) was added MeMgBr (3.0 M, 0.12 mL) at 0° C. After the addition, the reaction was stirred for 2 hours. The mixture was quenched with aq.NH₄Cl, extracted with EtOAc (10 mL×3). The combined organic layers were washed with brine, dried over Na₂SO₄ and concentrated. The resulting residue was purified by flash column chromatography to give the titled compound (23 mg, 32%). MS: M/e 406 (M+1)⁺.

### Step C: tert-butyl (2R,5S)-4-(2-(1-chloroethyl)-5-methyl-6-oxo-5,6-dihydroimidazo[1,2-b]pyridazin-8-yl)-2,5-dimethylpiperazine-1-carboxylate

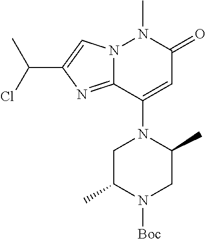 [SMILES: CC(Cl)c1cn2c(n1)c(N1C[C@@H](C)N(C(=O)OC(C)(C)C)C[C@@H]1C)cc(=O)n2C]

To a stirred solution of tert-butyl (2R,5S)-4-(2-(1-hydroxyethyl)-5-methyl-6-oxo-5,6-dihydroimidazo[1,2-b]pyridazin-8-yl)-2,5-dimethylpiperazine-1-carboxylate (23 mg, 0.056 mmol) in CH₂Cl₂ (5 mL) was added SOCl₂ (13.5 mg, 0.114 mmol), then the mixture was stirred for an hour. The reaction mixture was washed with aq.NaHCO₃, extracted with CH₂Cl₂ (5 mL×3). The combined organic layers were washed with brine, dried over Na₂SO₄, concentrated to give the titled compound which was directly used to the next step without further purification. MS: M/e 424 (M+1)⁺.

### Step D: tert-butyl (2R,5S)-4-(2-(1-methoxyethyl)-5-methyl-6-oxo-5,6-dihydroimidazo[1,2-b]pyridazin-8-yl)-2,5-dimethylpiperazine-1-carboxylate

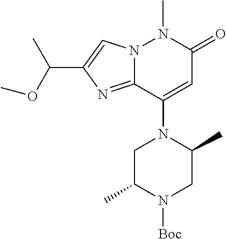

A mixture of tert-butyl (2R,5S)-4-(2-(1-chloroethyl)-5-methyl-6-oxo-5,6-dihydroimidazo[1,2-b]pyridazin-8-yl)-2,5-dimethylpiperazine-1-carboxylate (crude, 0.056 mmol) in MeOH (5 mL) was stirred at 60° C. overnight. The mixture was concentrated to give the titled compound (26 mg, crude) which was directly used to the next step without further purification. MS: M/e 420 (M+1)⁺.

### Step E: 8-((2S,5R)-2,5-dimethyl-4-(1-(quinoxalin-6-yl)ethyl)piperazin-1-yl)-2-(1-methoxyethyl)-5-methylimidazo[1,2-b]pyridazin-6(5H)-one

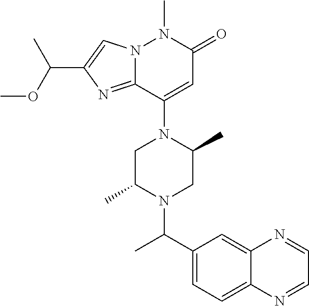 [SMILES: COC(C)c1cn2c(n1)c(N1C[C@@H](C)N(C(C)c3ccc4nccnc4c3)C[C@@H]1C)cc(=O)n2C]

To a stirred solution of tert-butyl (2R,5S)-4-(2-(1-methoxyethyl)-5-methyl-6-oxo-5,6-dihydroimidazo[1,2-b]pyridazin-8-yl)-2,5-dimethylpiperazine-1-carboxylate (26 mg, 0.056 mmol) in CH₂Cl₂ (5 mL) was added TFA (2 mL). Then the mixture was stirred for 2 hours. The mixture was concentrated to give the residue, which was washed with aq.NaHCO₃ and extracted with EtOAc (10 mL×2). The combined organic layers were washed with brine, dried over Na₂SO₄, concentrated to give the de-Boc intermediate, dissolved in CH₃CN (3 mL), 1-(quinoxalin-6-yl)ethan-1-ol (29.2 mg, 0.168 mmol), (cyanomethyl)trimethylphosphonium iodide (40.8 mg, 0.168 mmol) and DIPEA (90 mg, 0.7 mmol) were added. After the addition, the mixture was stirred at 100° C. overnight in a sealed tube. The reaction mixture was diluted with EtOAc (10 mL), washed with brine, dried over Na₂SO₄ and concentrated. The resulting residue was purified by Pre-TLC (CH₂Cl₂:MeOH=10:1) to give the titled compound (2 mg). ¹H NMR (400 MHz, DMSO-d₆) δ 8.93 (s, 2H), 8.15-7.92 (m, 3H), 7.83 (d, J=15.2 Hz, 1H), 5.48 (s, 1H), 4.42-4.31 (m, 1H), 3.98-3.76 (m, 1H), 3.62 (s, 3H), 3.60-3.48 (m, 1H), 3.26-3.15 (m, 3H), 3.01-2.64 (m, 3H), 2.19-1.94 (m, 2H), 1.51-1.30 (m, 8H), 1.13 (s, 3H), 1.01-0.93 (m, 1H) ppm. MS: M/e 476 (M+1)⁺.

### Compound A6: 2-(8-((2S,5R)-2,5-dimethyl-4-(1-(quinoxalin-6-yl)ethyl)piperazin-1-yl)-5-methyl-6-oxo-5,6-dihydroimidazo[1,2-b]pyridazin-2-yl)propanenitrile

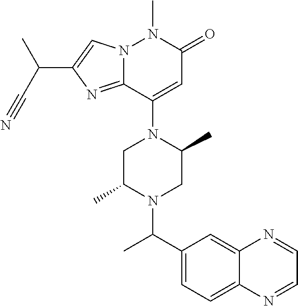 [SMILES: CC(C#N)c1cn2c(n1)c(N1C[C@@H](C)N(C(C)c3ccc4nccnc4c3)C[C@@H]1C)cc(=O)n2C]

### Step A: tert-butyl (2R,5S)-4-(2-(1-cyanoethyl)-5-methyl-6-oxo-5,6-dihydroimidazo[1,2-b]pyridazin-8-yl)-2,5-dimethylpiperazine-1-carboxylate

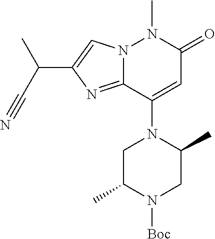 [SMILES: CC(C#N)c1cn2c(n1)c(N1C[C@@H](C)N(C(=O)OC(C)(C)C)C[C@@H]1C)cc(=O)n2C]

A mixture of tert-butyl (2R,5S)-4-(2-(1-chloroethyl)-5-methyl-6-oxo-5,6-dihydroimidazo[1,2-b]pyridazin-8-yl)-2,5-dimethylpiperazine-1-carboxylate (73 mg, 0.172 mmol), TMSCN (51 mg, 0.516 mmol) and Cs₂CO₃ (168 mg, 0.516 mmol) in CH₃CN (5 mL) was stirred at 70° C. overnight. The mixture was poured into H₂O (10 mL) and extracted with EtOAc (15 mL×2). The combined organic layers were washed with brine, dried over Na₂SO₄, and concentrated. The resulting residue was purified by Pre-TLC (EtOAc) to give the titled compound (15 mg, 21%). MS: M/e 415 (M+1)⁺.

### Step B: 2-(8-((2S,5R)-2,5-dimethyl-4-(1-(quinoxalin-6-yl)ethyl)piperazin-1-yl)-5-methyl-6-oxo-5,6-dihydroimidazo[1,2-b]pyridazin-2-yl)propanenitrile

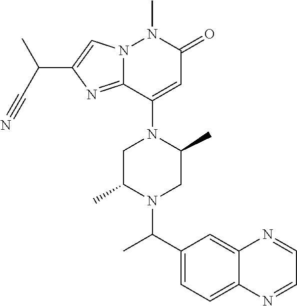 [SMILES: CC(C#N)c1cn2c(n1)c(N1C[C@@H](C)N(C(C)c3ccc4nccnc4c3)C[C@@H]1C)cc(=O)n2C]

To a stirred solution of tert-butyl (2R,5S)-4-(2-(1-cyanoethyl)-5-methyl-6-oxo-5,6-dihydroimidazo[1,2-b]pyridazin-8-yl)-2,5-dimethylpiperazine-1-carboxylate (15 mg, 0.036 mmol) in CH₂Cl₂ (5 mL) was added TFA (2 mL). Then the mixture was stirred for 2 hours. The mixture was concentrated to give the residue, which was washed with aq.NaHCO₃ and extracted with EtOAc (10 mL×2). The combined organic layers were washed with brine, dried over Na₂SO₄, concentrated to give the de-Boc intermediate, dissolved in CH₃CN (3 mL), 1-(quinoxalin-6-yl)ethan-1-ol (18.9 mg, 0.11 mmol), (cyanomethyl)trimethylphosphonium iodide (26.7 mg, 0.11 mmol) and DIPEA (47 mg, 0.36 mmol) were added. After the addition, the mixture was stirred at 100° C. overnight in a sealed tube. The reaction mixture was diluted with EtOAc (10 mL), washed with brine, dried over Na₂SO₄, and concentrated. The resulting residue was purified by Pre-TLC (CH₂Cl₂:MeOH=10:1) to give the titled compound (5 mg). ¹H NMR (400 MHz, DMSO-d₆) δ 8.97-8.90 (m, 2H), 8.15-7.92 (m, 3H), 5.69 (s, 1H), 4.27-3.93 (m, 1H), 3.88 (s, 3H), 3.83-3.75 (m, 1H), 3.64-3.49 (m, 1H), 2.99-2.77 (m, 2H), 2.78-2.64 (m, 3H), 2.14-1.94 (m, 2H), 1.70-1.43 (m, 2H), 1.40-1.32 (m, 5H), 1.13 (s, 3H), 1.02-0.95 (m, 1H) ppm. MS: M/e 471 (M+1)⁺.

### Compound A7: 8-((2S,5R)-2,5-dimethyl-4-(1-(quinoxalin-6-yl)ethyl)piperazin-1-yl)-2-((dimethylamino)methyl)-5-methylimidazo[1,2-b]pyridazin-6(5H)-one

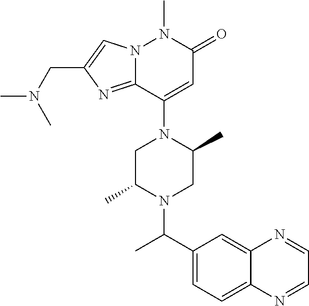 [SMILES: CC(c1ccc2nccnc2c1)N1C[C@H](C)N(c2cc(=O)n(C)n3cc(CN(C)C)nc23)C[C@H]1C]

### Step A: tert-butyl (2R,5S)-4-(2-((dimethylamino)methyl)-5-methyl-6-oxo-5,6-dihydroimidazo[1,2-b]pyridazin-8-yl)-2,5-dimethylpiperazine-1-carboxylate

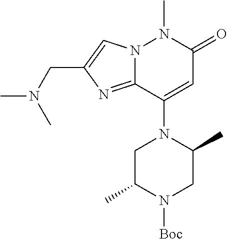

A mixture of tert-butyl (2R,5S)-4-(2-(chloromethyl)-5-methyl-6-oxo-5,6-dihydroimidazo[1,2-b]pyridazin-8-yl)-2,5-dimethylpiperazine-1-carboxylate (40.8 mg, 0.1 mmol) in a solution of dimethylamine in THF (2.0 M, 3 mL) was stirred for 3 hours. The reaction mixture was concentrated to give the titled compound, which was directly used to the next step without further purification. MS: M/e 419 (M+1)⁺.

### Step B: 8-((2S,5R)-2,5-dimethyl-4-(1-(quinoxalin-6-yl)ethyl)piperazin-1-yl)-2-((dimethylamino)methyl)-5-methylimidazo[1,2-b]pyridazin-6(5H)-one

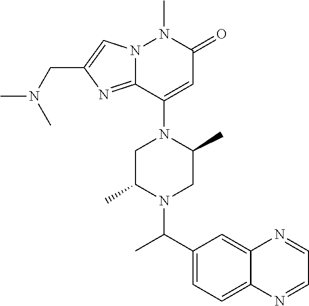 [SMILES: CC(c1ccc2nccnc2c1)N1C[C@H](C)N(c2cc(=O)n(C)n3cc(CN(C)C)nc23)C[C@H]1C]

To a stirred solution of tert-butyl (2R,5S)-4-(2-((dimethylamino)methyl)-5-methyl-6-oxo-5,6-dihydroimidazo[1,2-b]pyridazin-8-yl)-2,5-dimethylpiperazine-1-carboxylate (crude, 0.1 mmol) in CH₂Cl₂ (5 mL) was added TFA (1 mL). After stirred for 2 hours, TLC (CH₂Cl₂:MeOH=10:1) indicated the reaction was complete. The reaction mixture was concentrated to give the residue, which was dissolved in CH₃CN (5 mL), 6-(1-bromoethyl)quinoxaline (35.5 mg, 0.15 mmol) and DIPEA (51.6 mg, 0.4 mmol) were added. After the addition, the reaction mixture was stirred at 70° C. overnight. The reaction mixture was poured into H₂O (10 mL), extracted with EtOAc (10 mL×2), then the aqueous layer was concentrated to give the residue, which was purified by Prep-HPLC to give the titled compound (14 mg, 28%). ¹H NMR (400 MHz, CD₃OD) δ 9.00 (s, 2H), 8.53 (dd, J=16.0, 1.6 Hz, 1H), 8.27 (d, J=8.8 Hz, 1H), 8.20 (d, J=3.2 Hz, 1H), 8.12 (d, J=8.8 Hz, 1H), 5.87 (d, J=4.8 Hz, 1H), 5.22-5.14 (m, 1H), 5.07 (s, 1H), 4.80-4.63 (m, 3H), 3.88-3.72 (m, 5H), 3.69-3.60 (m, 1H), 3.25-3.15 (m, 4H), 3.06 (d, J=2.4 Hz, 3H), 2.10 (t, J=7.6 Hz, 3H), 1.47 (d, J=6.0 Hz, 3H), 1.42 (dd, J=6.8, 1.6 Hz, 3H) ppm. MS: M/e 475 (M+1)⁺.

### Compound A8: 8-((2S,5R)-2,5-diethyl-4-(1-(quinoxalin-6-yl)ethyl)piperazin-1-yl)-2-(methoxymethyl)-5-methylimidazo[1,2-b]pyridazin-6(5H)-one

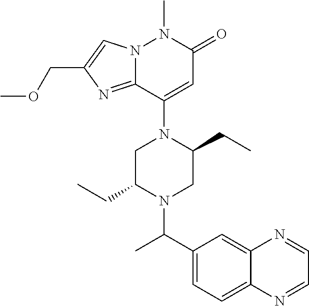 [SMILES: CC[C@H]1CN(C(C)c2ccc3nccnc3c2)[C@H](CC)CN1c1cc(=O)n(C)n2cc(COC)nc12]

### Step A: ethyl 8-((2S,5R)-4-benzyl-2,5-diethylpiperazin-1-yl)-6-chloroimidazo[1,2-b]pyridazine-2-carboxylate

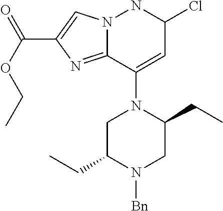 [SMILES: CCOC(=O)c1cn2c(n1)C(N1C[C@@H](CC)N(Cc3ccccc3)C[C@@H]1CC)=CC(Cl)N2]

A mixture of ethyl 8-bromo-6-chloroimidazo[1,2-b]pyridazine-2-carboxylate (2.5 g, 8.3 mmol), (2R,5S)-1-benzyl-2,5-diethylpiperazine (Intermediate 2)(2.0 g, 8.6 mmol) and DIPEA (3.2 g, 24.9 mmol) in MeCN (25 mL) was stirred at 90° C. for 16 hours. The mixture was diluted with EtOAc (25 mL), washed with brine (25 mL×3), dried and concentrated. The resulting residue was purified by flash column chromatography to give the titled compound (2.7 g, 71%). MS: M/e 456 (M+1)⁺.

### Step B: ethyl 8-((2S,5R)-4-benzyl-2,5-diethylpiperazin-1-yl)-6-oxo-5,6-dihydroimidazo[1,2-b]pyridazine-2-carboxylate

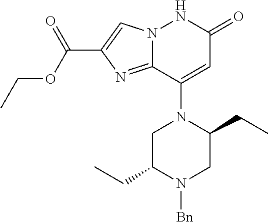 [SMILES: CCOC(=O)c1cn2[nH]c(=O)cc(N3C[C@@H](CC)N(Cc4ccccc4)C[C@@H]3CC)c2n1]

A mixture of ethyl 8-((2S,5R)-4-benzyl-2,5-diethylpiperazin-1-yl)-6-chloroimidazo[1,2-b]pyridazine-2-carboxylate (2.17 g, 4.79 mmol), Pd₂(dba)₃ (650 mg, 0.71 mmol), t-BuXPhos (600 mg, 1.41 mmol) and KOH (3M, 4.5 mmol) in 1,4-dioxane (25 mL) was stirred at 100° C. for 2 hours. The mixture was diluted with aqueous solution of NaHCO₃ (15 mL), extracted with EtOAc (20 mL×3). The combined organic layers was washed with brine (20 mL×2), dried and concentrated. The resulting residue was purified by flash column chromatography to give the titled compound (1.0 g, 48%). MS: M/e 438 (M+1)⁺.

### Step C: ethyl 8-((2S,5R)-4-benzyl-2,5-diethylpiperazin-1-yl)-5-methyl-6-oxo-5,6-dihydroimidazo[1,2-b]pyridazine-2-carboxylate

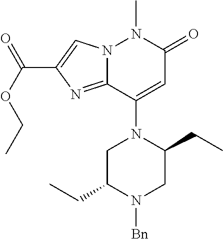 [SMILES: CCOC(=O)c1cn2c(n1)c(N1C[C@@H](CC)N(Cc3ccccc3)C[C@@H]1CC)cc(=O)n2C]

A mixture of ethyl 8-((2S,5R)-4-benzyl-2,5-diethylpiperazin-1-yl)-6-oxo-5,6-dihydroimidazo[1,2-b]pyridazine-2-carboxylate (1.0 g, 2.28 mmol), iodomethane (1.0 g, 7.04 mmol) and Cs₂CO₃ (2.3 g, 7.07 mmol) in Dioxane (10 mL) was stirred at 70° C. for 16 hours. The mixture was filtered and the filtrate was diluted with EtOAc (20 mL), washed with brine (10 mL×2), dried over Na₂SO₄, concentrated and purified by flash column chromatography to give the titled compound (540 mg, 52%). MS: M/e 452 (M+1)⁺.

### Step D: 8-((2S,5R)-4-benzyl-2,5-diethylpiperazin-1-yl)-2-(hydroxymethyl)-5-methylimidazo[1,2-b]pyridazin-6(5H)-one

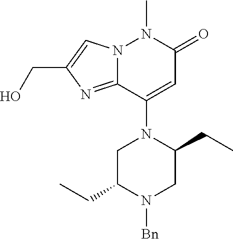 [SMILES: CC[C@@H]1CN(c2cc(=O)n(C)n3cc(CO)nc23)[C@@H](CC)CN1Cc1ccccc1]

To a stirred solution of ethyl 8-((2S,5R)-4-benzyl-2,5-diethylpiperazin-1-yl)-5-methyl-6-oxo-5,6-dihydroimidazo[1,2-b]pyridazine-2-carboxylate (540 mg, 1.2 mmol) in a mixed solvent THF/EtOH (1:1, 20 mL) was added NaBH₄ (273 mg, 7.2 mmol) in portions at room temperature. The resulted mixture was stirred at 60° C. for 3 days. The mixture was treated with saturated aqueous solution of NaHCO₃ (10 mL), extracted with EtOAc (20 mL×3). The combined organic layers was washed with brine (20 mL×3), dried over Na₂SO₄, concentrated. The resulted crude product was purified by flash column chromatography to give the titled compound (344 mg, 70%). MS: M/e 410 (M+1)⁺.

### Step E: tert-butyl (2R,5S)-2,5-diethyl-4-(2-(hydroxymethyl)-5-methyl-6-oxo-5,6-dihydroimidazo[1,2-b]pyridazin-8-yl)piperazine-1-carboxylate

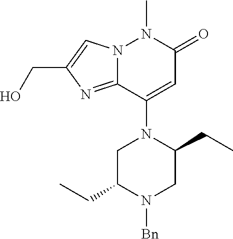 [SMILES: CC[C@@H]1CN(c2cc(=O)n(C)n3cc(CO)nc23)[C@@H](CC)CN1Cc1ccccc1]

A mixture of 8-((2S,5R)-4-benzyl-2,5-diethylpiperazin-1-yl)-2-(hydroxymethyl)-5-methylimidazo[1,2-b]pyridazin-6(5H)-one (340 mg, 0.83 mmol), Pd/C (100 mg) and di-tert-butyl dicarbonate (280 mg, 1.29 mmol) in MeOH (10 mL) was stirred at room temperature under H₂ for 16 hours. The mixture was filtered and the filtrate was concentrated. The resulted residue was purified by flash column chromatography to give the titled compound (280 mg, 80%). MS: M/e 420 (M+1)⁺.

### Step F: tert-butyl (2R,5S)-4-(2-(chloromethyl)-5-methyl-6-oxo-5,6-dihydroimidazo[1,2-b]pyridazin-8-yl)-2,5-diethylpiperazine-1-carboxylate

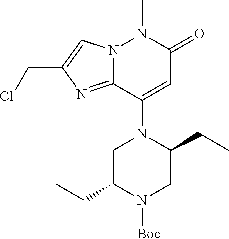 [SMILES: CC[C@@H]1CN(c2cc(=O)n(C)n3cc(CCl)nc23)[C@@H](CC)CN1C(=O)OC(C)(C)C]

To a stirred solution of tert-butyl (2R,5S)-2,5-diethyl-4-(2-(hydroxymethyl)-5-methyl-6-oxo-5,6-dihydroimidazo[1,2-b]pyridazin-8-yl)piperazine-1-carboxylate (280 mg, 0.67 mmol) in CH₂Cl₂ (5 mL) was added SOCl₂ (160 mg, 1.33 mmol). The resulted mixture was stirred at room temperature for 1 hour. The mixture was concentrated to dryness and treated with saturated aqueous solution of NaHCO₃ (5 mL), extracted with DCM (5 ml×3). The combined organic layers was washed with brine (10 mL×2), dried over Na₂SO₄, concentrated. The resulted crude product was purified by flash column chromatography to give the titled compound (270 mg, 92%). MS: M/e 438 (M+1)⁺.

### Step G: tert-butyl (2R,5S)-2,5-diethyl-4-(2-(methoxymethyl)-5-methyl-6-oxo-5,6-dihydroimidazo[1,2-b]pyridazin-8-yl)piperazine-1-carboxylate

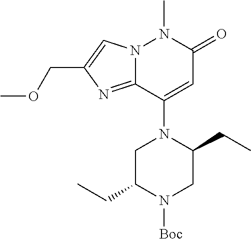 [SMILES: CC[C@@H]1CN(c2cc(=O)n(C)n3cc(COC)nc23)[C@@H](CC)CN1C(=O)OC(C)(C)C]

tert-butyl (2R,5S)-4-(2-(chloromethyl)-5-methyl-6-oxo-5,6-dihydroimidazo[1,2-b]pyridazin-8-yl)-2,5-diethylpiperazine-1-carboxylate (120 mg, 0.27 mmol) was added into a solution of CH₃ONa in MeOH (5.4 M, 4 mL) and the mixture was stirred at 70° C. for 3 hours. The mixture was diluted with EtOAc (20 mL), washed with brine (5 mL×3), dried over Na₂SO₄, concentrated and purified by flash column chromatography to give the titled compound (100 mg, 83%). MS: M/e 434 (M+1)⁺.

### Step H: 8-((2S,5R)-2,5-diethylpiperazin-1-yl)-2-(methoxymethyl)-5-methylimidazo[1,2-b]pyridazin-6(5H)-one

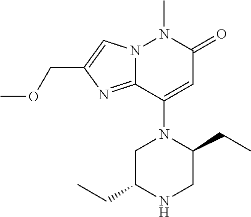 [SMILES: CC[C@@H]1CN(c2cc(=O)n(C)n3cc(COC)nc23)[C@@H](CC)CN1]

To a stirred solution of tert-butyl (2R,5S)-2,5-diethyl-4-(2-(methoxymethyl)-5-methyl-6-oxo-5,6-dihydroimidazo[1,2-b]pyridazin-8-yl)piperazine-1-carboxylate (100 mg, 0.23 mmol) in CH₂Cl₂ (5 mL) was added TFA (1 mL). The resulted mixture was stirred at room temperature for 3 hours. The mixture was concentrated and the resulted residue was treated with saturated aqueous solution of NaHCO₃ to pH>8. The mixture was extracted with DCM (5 mL×3). The combined organic layers was washed with brine (5 mL×2), dried over Na₂SO₄ and concentrated to give the titled compound (35 mg, 45%). MS: M/e 334 (M+1)⁺.

### Step I: 8-((2S,5R)-2,5-diethyl-4-(1-(quinoxalin-6-yl)ethyl)piperazin-1-yl)-2-(methoxymethyl)-5-methylimidazo[1,2-b]pyridazin-6(5H)-one

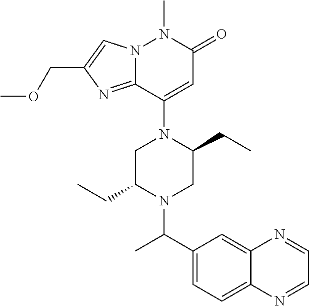 [SMILES: CC[C@H]1CN(C(C)c2ccc3nccnc3c2)[C@H](CC)CN1c1cc(=O)n(C)n2cc(COC)nc12]

A mixture of 8-((2S,5R)-2,5-diethylpiperazin-1-yl)-2-(methoxymethyl)-5-methylimidazo[1,2-b]pyridazin-6(5H)-one (35 mg, 0.1 mmol), 1-(quinoxalin-6-yl)ethan-1-ol (55 mg, 0.3 mmol), (cyanomethyl)trimethylphosphonium iodide (100 mg, 0.41 mmol) and DIPEA (150 mg, 1.16 mmol) in MeCN (1 mL) was stirred at 100° C. for 16 hours. The resulted mixture was diluted with EtOAc (5 mL), washed with brine (2 mL×3), dried and concentrated. The resulting residue was purified by flash column chromatography to give the titled Compound A8 which was separated into Compound A8a (6 mg) and Compound A8b (7 mg) by Prep-HPLC (Method B).

Compound A8a (the earlier peak): ¹H NMR (400 MHz, CD₃OD) δ 8.88 (dd, J=4.4, 1.6 Hz, 2H), 8.12 (d, J=8.4 Hz, 1H), 8.05 (s, 1H), 8.00 (dd, J=8.8, 1.6 Hz, 1H), 7.79 (s, 1H), 5.61 (s, 1H), 4.47 (s, 2H), 4.07 (q, J=6.4 Hz, 1H), 3.74 (s, 3H), 3.40-3.31 (m, 6H), 3.15-3.03 (m, 1H), 3.02-2.92 (m, 1H), 2.50-2.35 (m, 1H), 2.24-2.04 (m, 1H), 1.97-1.81 (m, 1H), 1.70-1.52 (m, 2H), 1.46 (d, J=6.4 Hz, 3H), 0.94 (t, J=7.2 Hz, 3H), 0.66 (t, J=7.2 Hz, 3H). MS: M/e 490 (M+1)⁺.

Compound A8a (the later peak): ¹H NMR (400 MHz, CD₃OD) δ 8.91-8.83 (m, 2H), 8.14-8.05 (m, 2H), 8.02 (d, J=8.8 Hz, 1H), 7.79 (s, 1H), 5.60 (s, 1H), 4.48 (s, 2H), 3.92 (q, J=6.4 Hz, 1H), 3.74 (s, 3H), 3.56 (d, J=12.8 Hz, 1H), 3.39 (s, 3H), 3.30-3.29 (m, 2H), 3.23 (d, J=10.8 Hz, 1H), 2.77 (d, J=12.8 Hz, 1H), 2.36 (d, J=12.0 Hz, 1H), 2.06-1.86 (m, 1H), 1.80-1.66 (m, 2H), 1.65-1.55 (m, 1H), 1.43 (d, J=6.8 Hz, 3H), 1.03 (t, J=7.2 Hz, 3H), 0.55 (t, J=7.2 Hz, 3H). MS: M/e 490 (M+1)⁺.

Compound A9: ((2S,5R)-5-ethyl-2-methyl-4-(1-(quinoxalin-6-yl)ethyl)piperazin-1-yl)-5-methyl-6-oxo-5,6-dihydroimidazo[1,2-b]pyridazine-2-carbaldehyde O-methyl oxime

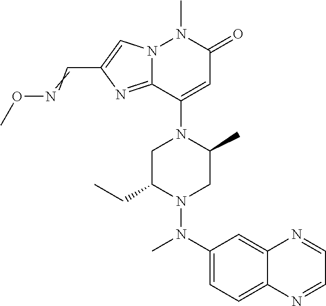 [SMILES: CC[C@@H]1CN(c2cc(=O)n(C)n3cc(C=NOC)nc23)[C@@H](C)CN1N(C)c1ccc2nccnc2c1]

### Step A: tert-butyl (2R,5S)-2-ethyl-4-(2-formyl-5-methyl-6-oxo-5,6-dihydroimidazo[1,2-b]pyridazin-8-yl)-5-methylpiperazine-1-carboxylate

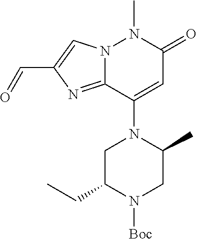 [SMILES: CC[C@@H]1CN(c2cc(=O)n(C)n3cc(C=O)nc23)[C@@H](C)CN1C(=O)OC(C)(C)C]

To a solution of tert-butyl (2R,5S)-2-ethyl-4-(2-(hydroxymethyl)-5-methyl-6-oxo-5,6-dihydroimidazo[1,2-b]pyridazin-8-yl)-5-methylpiperazine-1-carboxylate (200 mg, 0.5 mmol) in DCM (10 mL) was added MnO₂ (2.0 g, 23 mmol) at room temperature and the mixture was stirred at room temperature for 16 hours. The mixture was filtered and the filtrate was concentrated to dryness to give the titled compound (180 mg, 90%). MS: M/e 404 (M+1)⁺.

### Step B: tert-butyl (2R,5S)-2-ethyl-4-(2-((methoxyimino)methyl)-5-methyl-6-oxo-5,6-dihydroimidazo[1,2-b]pyridazin-8-yl)-5-methylpiperazine-1-carboxylate

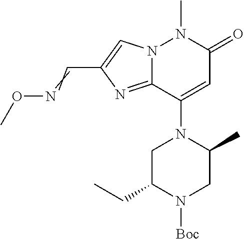 [SMILES: CC[C@@H]1CN(c2cc(=O)n(C)n3cc(C=NOC)nc23)[C@@H](C)CN1C(=O)OC(C)(C)C]

A mixture of tert-butyl (2R,5S)-2-ethyl-4-(2-formyl-5-methyl-6-oxo-5,6-dihydroimidazo[1,2-b]pyridazin-8-yl)-5-methylpiperazine-1-carboxylate (120 mg, 0.3 mmol), O-methylhydroxylamine hydrochloride (120 mg, 1.4 mmol) and CH₃COONa (150 mg, 1.8 mmol) in a mixed solvent H₂O/THF (1/2, 5 mL) was stirred at room temperature for 20 hours. The mixture was diluted with EtOAc (20 mL), washed with brine (10 mL×2), dried over Na₂SO₄ and concentrated. The resulting residue was purified by flash column chromatography to give the titled compound (100 mg, 77%). MS: M/e 433 (M+1)⁺.

### Step C: 8-((2S,5R)-5-ethyl-2-methylpiperazin-1-yl)-5-methyl-6-oxo-5,6-dihydroimidazo[1,2-b]pyridazine-2-carbaldehyde O-methyl oxime

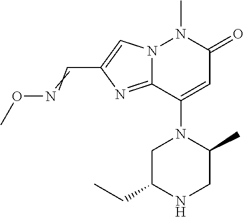 [SMILES: CC[C@@H]1CN(c2cc(=O)n(C)n3cc(C=NOC)nc23)[C@@H](C)CN1]

To a solution of tert-butyl (2R,5S)-2-ethyl-4-(2-(methoxyimino)methyl)-5-methyl-6-oxo-5,6-dihydroimidazo[1,2-b]pyridazin-8-yl)-5-methylpiperazine-1-carboxylate (80 mg, 0.185 mmol) in DCM was added TFA (2 mL) and the mixture was stirred at room temperature for 2 hours. The mixture was concentrated, treated with saturated aqueous solution of NaHCO₃ (2 mL), extracted with DCM (2 mL×3). The combined organic layers was washed with brine (2 mL×2), dried over Na₂SO₄, and concentrated to give the titled compound (55 mg, 89%). MS: M/e 333 (M+1)⁺.

### Step D: ((2S,5R)-5-ethyl-2-methyl-4-(1-(quinoxalin-6-yl)ethyl)piperazin-1-yl)-5-methyl-6-oxo-5,6-dihydroimidazo[1,2-b]pyridazine-2-carbaldehyde O-methyl oxime

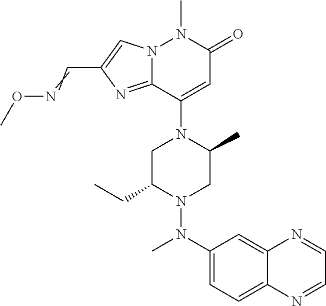 [SMILES: CC[C@@H]1CN(c2cc(=O)n(C)n3cc(C=NOC)nc23)[C@@H](C)CN1N(C)c1ccc2nccnc2c1]

A mixture of 8-((2S,5R)-5-ethyl-2-methylpiperazin-1-yl)-5-methyl-6-oxo-5,6-dihydroimidazo[1,2-b]pyridazine-2-carbaldehyde O-methyl oxime (30 mg, 0.09 mmol), 1-(quinoxalin-6-yl)ethan-1-ol (45 mg, 0.26 mmol), (cyanomethyl)trimethylphosphonium iodide (65 mg, 0.26 mmol) and DIPEA (75 mg, 0.58 mmol) in MeCN (1 mL) was stirred at 100° C. for 16 hours. The resulted mixture was diluted with EtOAc (10 mL), washed with brine (5 mL×3), dried and concentrated. The resulting residue was purified by flash column chromatography to give the titled Compound A9 which was separated into Compound A9a (3 mg), Compound A9b (7 mg), Compound A9c (3 mg) and Compound A9d (5 mg) by Prep-HPLC (Method B).

Compound A9a (the first peak)). ¹H NMR (400 MHz, CD₃OD) δ 8.88 (dd, J=5.2, 2.0 Hz, 2H), 8.12 (d, J=8.4 Hz, 1H), 8.06 (d, J=7.2 Hz, 2H), 8.04-7.99 (m, 2H), 5.65 (s, 1H), 4.12-4.04 (m, 1H), 3.89 (s, 3H), 3.75 (s, 3H), 3.38-3.32 (m, 2H), 3.06 (dd, J=11.6, 4.0 Hz, 1H), 2.97-2.89 (m, 1H), 2.88-2.66 (m, 1H), 2.52-2.39 (m, 1H), 1.69-1.58 (m, 2H), 1.50-1.39 (m, 6H), 0.71 (t, J=7.2 Hz, 3H). MS: M/e 489 (M+1)⁺.

Compound A9b (the second peak)). ¹H NMR (400 MHz, CD₃OD) δ 8.88 (dd, J=5.2, 1.6 Hz, 2H), 8.29 (s, 1H), 8.12 (d, J=8.8 Hz, 1H), 8.08-7.99 (m, 2H), 7.39 (s, 1H), 5.67 (s, 1H), 4.14-4.01 (m, 4H), 3.77 (s, 3H), 3.39-3.32 (m, 2H), 3.05 (dd, J=11.6, 3.6 Hz, 1H), 2.93 (d, J=12.0 Hz, 1H), 2.89-2.61 (m, 1H), 2.51-2.41 (m, 1H), 1.72-1.57 (m, 2H), 1.52-1.40 (m, 6H), 0.73 (t, J=7.2 Hz, 3H). MS: M/e 489 (M+1)⁺.

Compound A9c: (the third peak)). ¹H NMR (400 MHz, CD₃OD) δ 8.92-8.84 (m, 2H), 8.16-7.98 (m, 5H), 5.64 (s, 1H), 3.99-3.85 (m, 4H), 3.75 (s, 3H), 3.64-3.54 (m, 1H), 3.42-3.32 (m, 1H), 3.28-3.21 (m, 1H), 2.96-2.67 (m, 2H), 2.26 (d, J=10.8 Hz, 1H), 1.84-1.69 (m, 1H), 1.68-1.55 (m, 1H), 1.45 (d, J=6.4 Hz, 3H), 1.23 (d, J=6.8 Hz, 3H), 1.07 (t, J=7.2 Hz, 3H). MS: M/e 489 (M+1)⁺.

Compound A9d (the fourth peak): ¹H NMR (400 MHz, CD₃OD) δ 8.87 (dd, J=6.0, 1.6 Hz, 2H), 8.30 (s, 1H), 8.12-8.06 (m, 2H), 8.06-8.01 (m, 1H), 7.43 (s, 1H), 5.65 (s, 1H), 4.06 (s, 3H), 3.94 (q, J=6.4 Hz, 1H), 3.77 (s, 3H), 3.59 (d, J=11.2 Hz, 1H), 3.34-3.31 (m, 1H), 3.24 (d, J=10.4 Hz, 1H), 2.93-2.64 (m, 2H), 2.27 (d, J=12.0 Hz, 1H), 1.84-1.69 (m, 1H), 1.69-1.57 (m, 1H), 1.45 (d, J=6.4 Hz, 3H), 1.24 (d, J=6.4 Hz, 3H), 1.09 (t, J=7.2 Hz, 3H). MS: M/e 489 (M+1)⁺.

Compound A10: 8-((2S,5R)-2,5-dimethyl-4-(1-(quinoxalin-6-yl)ethyl)piperazin-1-yl)-5-methyl-2-((methylsulfinyl)methyl)imidazo[1,2-b]pyridazin-6(5H)-one

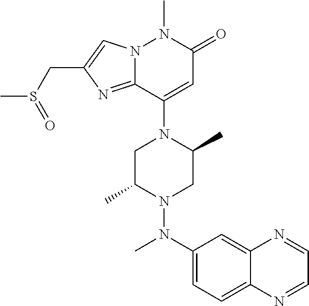 [SMILES: C[C@@H]1CN(c2cc(=O)n(C)n3cc(CS(C)=O)nc23)[C@@H](C)CN1N(C)c1ccc2nccnc2c1]

### Step A: tert-butyl (2R,5S)-2,5-dimethyl-4-(5-methyl-2-((methylthio)methyl)-6-oxo-5,6-dihydroimidazo[1,2-b]pyridazin-8-yl)piperazine-1-carboxylate

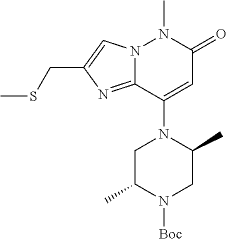 [SMILES: CSCc1cn2c(n1)c(N1C[C@@H](C)N(C(=O)OC(C)(C)C)C[C@@H]1C)cc(=O)n2C]

To a stirred solution of tert-butyl (2R,5S)-4-(2-(chloromethyl)-5-methyl-6-oxo-5,6-dihydroimidazo[1,2-b]pyridazin-8-yl)-2,5-dimethylpiperazine-1-carboxylate (40.9 mg, 0.1 mmol) in THF (5 mL) was added Et₃N (101 mg, 1 mmol), then sodium methyl mercaptide (20% in H₂O, 0.5 mL) was added. After the addition, the reaction mixture was stirred overnight. The reaction mixture was poured into H₂O (20 mL) and extracted with EtOAc (20 mL×2). The combined organic layers were washed with brine, dried over Na₂SO₄, concentrated to give the titled compound, which was directly used to the next step without further purification. MS: M/e 422 (M+1)⁺.

### Step B: tert-butyl (2R,5S)-2,5-dimethyl-4-(5-methyl-2-((methylsulfinyl)methyl)-6-oxo-5,6-dihydroimidazo[1,2-b]pyridazin-8-yl)piperazine-1-carboxylate

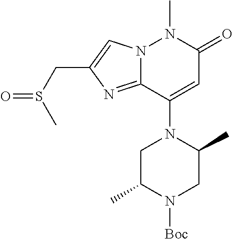 [SMILES: C[C@@H]1CN(c2cc(=O)n(C)n3cc(CS(C)=O)nc23)[C@@H](C)CN1C(=O)OC(C)(C)C]

To a stirred solution of tert-butyl (2R,5S)-2,5-dimethyl-4-(5-methyl-2-((methylthio)methyl)-6-oxo-5,6-dihydroimidazo[1,2-b]pyridazin-8-yl)piperazine-1-carboxylate (crude, 0.1 mmol) in CH₂Cl₂ (5 mL) was added m-CPBA (25.8 mg, 0.15 mmol). After stirred for 2 hours, the reaction mixture was washed with NaHCO₃ aq., brine, dried over Na₂SO₄ and concentrated. The resulting residue was purified by Pre-TLC (CH₂Cl₂:MeOH=10:1) to give the titled compound (26 mg, 60%). MS: M/e 438 (M+1)⁺.

### Step C: 8-((2S,5R)-2,5-dimethyl-4-(1-(quinoxalin-6-yl)ethyl)piperazin-1-yl)-5-methyl-2-((methylsulfinyl)methyl)imidazo[1,2-b]pyridazin-6(5H)-one

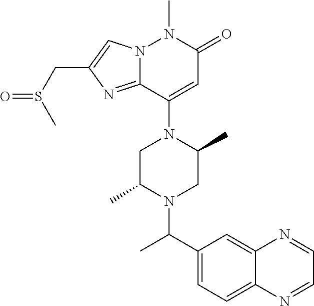 [SMILES: CC(c1ccc2nccnc2c1)N1C[C@H](C)N(c2cc(=O)n(C)n3cc(CS(C)=O)nc23)C[C@H]1C]

To a stirred solution of tert-butyl (2R,5S)-2,5-dimethyl-4-(5-methyl-2-((methylsulfinyl)methyl)-6-oxo-5,6-dihydroimidazo[1,2-b]pyridazin-8-yl)piperazine-1-carboxylate (26 mg, 0.06 mmol) in CH₂Cl₂ (5 mL) was added TFA (1 mL). After the addition, the reaction mixture was stirred for 4 hours. The reaction mixture was concentrated to give the residue, which was dissolved in EtOAc (20 mL) and washed with aq.Na₂CO₃, brine, dried over Na₂SO₄, concentrated to give the de-Boc intermediate, which was dissolved in CH₃CN (3 mL), and 1-(quinoxalin-6-yl)ethan-1-ol (31.3 mg, 0.18 mmol), (cyanomethyl)trimethylphosphonium iodide (43.7 mg, 0.18 mmol), DIPEA (77.4 mg, 0.6 mmol) were added. After the addition, the reaction mixture was stirred at 100° C. overnight in a sealed tube. The reaction mixture was poured into H₂O (10 mL), extracted with EtOAc (10 mL×2). The combined organic layers were washed with brine, dried over Na₂SO₄, and concentrated. The resulting residue was purified by Pre-TLC (CH₂Cl₂:MeOH=10:1) to give the titled compound (4 mg). H NMR (400 MHz, CD₃OD) δ 8.91-8.83 (m, 2H), 8.14-8.00 (m, 3H), 7.86-7.81 (m, 1H), 5.63 (s, 1H), 5.22-4.39 (m, 2H), 4.28-4.04 (m, 2H), 4.02-3.79 (m, 1H), 3.75 (s, 3H), 3.72-3.65 (m, 1H), 3.50-3.42 (m, 0.5H), 3.14-2.83 (m, 2H), 2.68-2.60 (m, 3H), 2.21 (d, J=12 Hz, 0.5H), 1.52-1.40 (m, 4.5H), 1.27-1.17 (m, 3H), 1.06 (d, J=6.4 Hz, 1.5H) ppm. MS: M/e 494 (M+1)⁺

### Compound A11: 8-((2S,5R)-2,5-dimethyl-4-(1-(quinoxalin-6-yl)ethyl)piperazin-1-yl)-5-methyl-2-((methylsulfonyl)methyl)imidazo[1,2-b]pyridazin-6(5H)-one

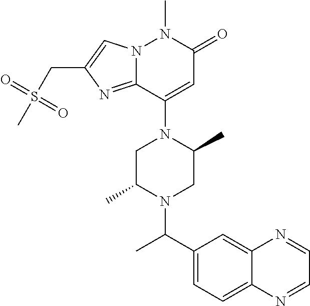 [SMILES: CC(c1ccc2nccnc2c1)N1C[C@H](C)N(c2cc(=O)n(C)n3cc(CS(C)(=O)=O)nc23)C[C@H]1C]

### Step A: tert-butyl (2R,5S)-2,5-dimethyl-4-(5-methyl-2-((methylsulfonyl)methyl)-6-oxo-5,6-dihydroimidazo[1,2-b]pyridazin-8-yl)piperazine-1-carboxylate

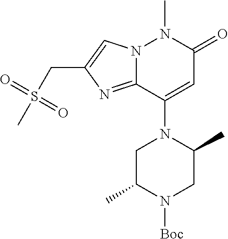 [SMILES: C[C@@H]1CN(c2cc(=O)n(C)n3cc(CS(C)(=O)=O)nc23)[C@@H](C)CN1C(=O)OC(C)(C)C]

To a stirred solution of tert-butyl (2R,5S)-2,5-dimethyl-4-(5-methyl-2-((methylthio)methyl)-6-oxo-5,6-dihydroimidazo[1,2-b]pyridazin-8-yl)piperazine-1-carboxylate (63 mg, 0.15 mmol) in CH₂Cl₂ (15 mL) was added m-CPBA (78 mg, 0.45 mmol). After the addition, the reaction was stirred for 2 days. The reaction mixture was washed with aq.Na₂CO₃, brine, dried over Na₂SO₄ and concentrated. The resulting residue was purified by Pre-TLC (CH₂Cl₂:MeOH=10:1) to give the titled compound (8 mg, 12%). MS: M/e 454 (M+1)⁺.

### Step B: 8-((2S,5R)-2,5-dimethyl-4-(1-(quinoxalin-6-yl)ethyl)piperazin-1-yl)-5-methyl-2-((methylsulfonyl)methyl)imidazo[1,2-b]pyridazin-6(5H)-one

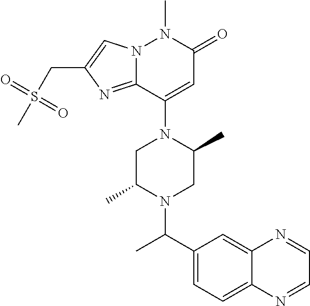 [SMILES: CC(c1ccc2nccnc2c1)N1C[C@H](C)N(c2cc(=O)n(C)n3cc(CS(C)(=O)=O)nc23)C[C@H]1C]

To a stirred solution of tert-butyl (2R,5S)-2,5-dimethyl-4-(5-methyl-2-((methylsulfonyl)methyl)-6-oxo-5,6-dihydroimidazo[1,2-b]pyridazin-8-yl)piperazine-1-carboxylate (8 mg, 0.018 mmol) in CH₂Cl₂ (5 mL) was added TFA (1 mL). After the addition, the reaction mixture was stirred for 4 hours. The reaction mixture was concentrated to give the residue, which was treated with aq.Na₂CO₃, and extracted with CH₂Cl₂:IPA (3:1, 10 mL×3). The combined organic layers were washed with brine, dried over Na₂SO₄, concentrated to give the de-Boc intermediate, which was dissolved in CH₃CN (3 mL), and 1-(quinoxalin-6-yl)ethan-1-ol (9.2 mg, 0.053 mmol), (cyanomethyl)trimethylphosphonium iodide (12.8 mg, 0.053 mmol), DIPEA (23.22 mg, 0.18 mmol) were added. After the addition, the reaction mixture was stirred at 100° C. overnight in a sealed tube. The reaction mixture was poured into H₂O (10 mL), extracted with EtOAc (10 mL×2). The combined organic layers were washed with brine, dried over Na₂SO₄, and concentrated. The resulting residue was purified by Pre-TLC (CH₂Cl₂:MeOH=10:1) to give the titled compound (4 mg). ¹H NMR (400 MHz, CD₃OD) δ 9.06-8.82 (m, 2H), 8.19-8.00 (m, 3H), 7.92 (s, 1H), 5.66 (s, 1H), 4.48 (s, 2H), 3.80-3.68 (m, 5H), 3.27-3.19 (m, 2H), 2.98 (s, 3H), 1.52-1.42 (m, 3H), 1.39-1.35 (m, 9H) ppm. MS: M/e 510 (M+1)⁺.

### Compound A12: 8-((2S,5R)-2,5-dimethyl-4-(1-(quinoxalin-6-yl)ethyl)piperazin-1-yl)-5-methyl-2-((S-methylsulfonimidoyl)methyl)imidazo[1,2-b]pyridazin-6(5H)-one

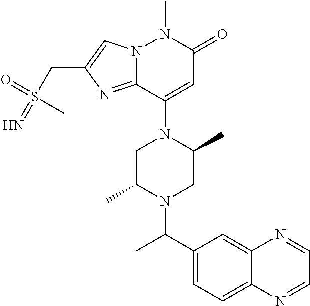 [SMILES: CC(c1ccc2nccnc2c1)N1C[C@H](C)N(c2cc(=O)n(C)n3cc(CS(C)(=N)=O)nc23)C[C@H]1C]

### Step A: N-(((8-((2S,5R)-2,5-dimethyl-4-(1-(quinoxalin-6-yl)ethyl)piperazin-1-yl)-5-methyl-6-oxo-5,6-dihydroimidazo[1,2-b]pyridazin-2-yl)methyl)(methyl)(oxo)-16-sulfaneylidene)-2,2,2-trifluoroacetamide

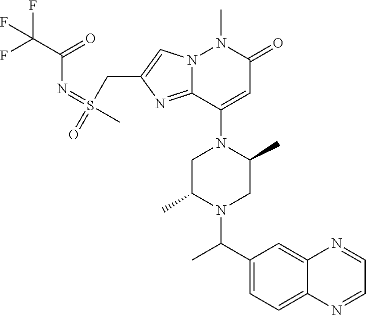 [SMILES: CC(c1ccc2nccnc2c1)N1C[C@H](C)N(c2cc(=O)n(C)n3cc(CS(C)(=O)=NC(=O)C(F)(F)F)nc23)C[C@H]1C]

To a stirred mixture of 8-((2S,5R)-2,5-dimethyl-4-(1-(quinoxalin-6-yl)ethyl)piperazin-1-yl)-5-methyl-2-((methylsulfinyl)methyl)imidazo[1,2-b]pyridazin-6(5H)-one (69 mg, 0.14 mmol), PhI (OAc)₂ (69 mg, 0.21 mmol), MgO (22.4 mg, 0.54 mmol) and Rh₂(OAc)₄ (6.2 mg, 0.014 mmol) in CH₂Cl₂ (10 mL) was added 2,2,2-trifluoroacetamide (31.6 mg, 0.28 mmol) under N₂. After the addition, the reaction mixture was stirred for 2 days. The reaction mixture was filtered and the filtrated was concentrated, purified by Pre-TLC (CH₂Cl₂:MeOH=10:1) to give the titled compound (14 mg, 17%). MS: M/e 605 (M+1)⁺.

### Step B: 8-((2S,5R)-2,5-dimethyl-4-(1-(quinoxalin-6-yl)ethyl)piperazin-1-yl)-5-methyl-2-((S-methylsulfonimidoyl)methyl)imidazo[1,2-b]pyridazin-6(5H)-one

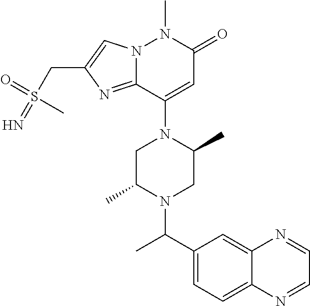 [SMILES: CC(c1ccc2nccnc2c1)N1C[C@H](C)N(c2cc(=O)n(C)n3cc(CS(C)(=N)=O)nc23)C[C@H]1C]

To a stirred solution of N-(((8-((2S,5R)-2,5-dimethyl-4-(1-(quinoxalin-6-yl)ethyl)piperazin-1-yl)-5-methyl-6-oxo-5,6-dihydroimidazo[1,2-b]pyridazin-2-yl)methyl)(methyl)(oxo)-16-sulfaneylidene)-2,2,2-trifluoroacetamide (14 mg, 0.023 mmol) in MeOH (5 mL) was added K₂CO₃ (16 mg, 0.12 mmol). After the addition, the reaction mixture was stirred for another 30 min. The reaction mixture was concentrated to give the residue, which was purified by Prep-HPLC to give the titled compound (0.8 mg). ¹H NMR (400 MHz, CD₃OD) δ 8.91-8.84 (m, 2H), 8.13-8.03 (m, 3H), 7.90-7.86 (m, 1H), 5.64 (s, 1H), 4.63-4.45 (m, 2H), 4.07-3.93 (m, 1H), 3.88-3.78 (m, 1H), 3.75 (s, 3H), 3.73-3.65 (m, 2H), 3.02 (d, J=2.8 Hz, 3H), 2.97-2.72 (m, 3H), 2.21 (d, J=10.4 Hz, 1H), 1.50-1.41 (m, 4.5H), 1.28-1.17 (m, 4.5H) ppm. MS: M/e 509 (M+1)⁺.

### Compound A13: 2-(8-((2S,5R)-5-ethyl-2-methyl-4-(1-(quinoxalin-6-yl)ethyl)piperazin-1-yl)-5-methyl-6-oxo-5,6-dihydroimidazo[1,2-b]pyridazin-2-yl)acetonitrile

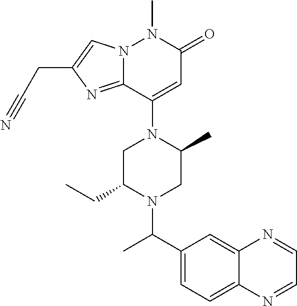 [SMILES: CC[C@@H]1CN(c2cc(=O)n(C)n3cc(CC#N)nc23)[C@@H](C)CN1C(C)c1ccc2nccnc2c1]

### Step A: methyl benzyl-L-alaninate

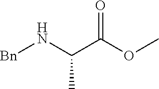

To a suspension of methyl L-alaninate hydrogen chloride (50 g, 0.36 mol) and K₂CO₃ (150 g, 1.08 mol) in MeCN (500 mL) was added BnBr (80 g, 0.47 mol) in drops at room temperature and the resulted mixture was stirred at room temperature for 16 hours. The mixture was filtered. The filter cake was washed with EtOAc (200 mL×3). The organics were combined and concentrated. The resulted residue was diluted with EtOAc (500 mL), washed with saturated aqueous solution of NaHCO₃ (200 mL), brine (200 mL×3), dried and concentrated. The resulted oil was purified by flash column chromatography to give the titled compound (28.0 g, 40%). MS: M/e 194 (M+1)⁺.

### Step B: methyl N-benzyl-N—((R)-2-((tert-butoxycarbonyl)amino)butanoyl)-L-alaninate

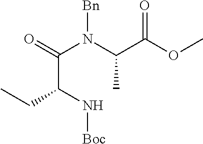 [SMILES: CC[C@@H](NC(=O)OC(C)(C)C)C(=O)N(Cc1ccccc1)[C@@H](C)C(=O)OC]

To a mixture of methyl benzyl-L-alaninate (27.0 g, 140 mmol), (R)-2-((tert-butoxycarbonyl)amino)butanoic acid (31.2 g, 154 mmol) and DIPEA (36.5 g, 280 mmol) in CH₂Cl₂ (300 mL) was added HATU (60.0 g, 158 mmol) in portions at 0° C. The resulted mixture was stirred at room temperature for 20 hours. The mixture was washed with saturated aqueous solution of NaHCO₃ (100 mL×2), brine (100 mL×2), dried over Na₂SO₄ and concentrated. The residue was purified by flash column chromatography to give the titled compound (45 g, 85%). MS: M/e 379 (M+1)⁺.

### Step C: methyl N—((R)-2-aminobutanoyl)-N-benzyl-L-alaninate

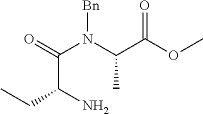 [SMILES: CC[C@@H](N)C(=O)N(Cc1ccccc1)[C@@H](C)C(=O)OC]

To a solution of methyl N-benzyl-N—((R)-2-((tert-butoxycarbonyl)amino)butanoyl)-L-alaninate (41.0 g, 108.5 mmol) in EtOAc (300 mL) was added HCl (100 mL, 4M in Dioxane) at room temperature and the mixture was stirred at room temperature for 3 days. The mixture was concentrated to dryness to give the titled compound (38.0 g, crude) which was used for the next step directly. MS: M/e 279 (M+1)⁺.

### Step D: (3R,6S)-1-benzyl-3-ethyl-6-methylpiperazine-2,5-dione

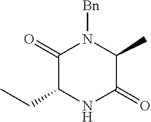 [SMILES: CC[C@H]1NC(=O)[C@H](C)N(Cc2ccccc2)C1=O]

To a mixture of methyl N—((R)-2-aminobutanoyl)-N-benzyl-L-alaninate hydrogen chloride (38.0 g, crude) in a mixed solvent EA/H₂O (200 mL/50 mL) was added NaHCO₃ (55 g, 650 mmol) in portions at room temperature. The resulted mixture was stirred at room temperature for 5 hours. Layers were separated. The aqueous layer was extracted with EtOAc (100 mL×3). The combined organic layers was washed with brine (200 mL×3), dried over Na₂SO₄ and concentrated. The resulting residue was purified by flash column chromatography to give the titled compound (24 g, 88% for 2 steps). MS: M/e 247 (M+1)⁺.

### Step E: (2S,5R)-1-benzyl-5-ethyl-2-methylpiperazine

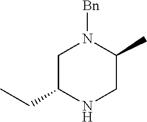 [SMILES: CC[C@@H]1CN(Cc2ccccc2)[C@@H](C)CN1]

A solution of borane in THF (1M, 480 mL) was slowly added to (3R,6S)-1-benzyl-3-ethyl-6-methylpiperazine-2,5-dione (23.6 g, 95.9 mmol) over 15 min. The resulted mixture was stirred at 70° C. for 72 hours. The mixture was cooled to 0° C., MeOH (100 mL) was added slowly and followed by HCl (5M, 40 mL) slowly. Large amount of bubble was found. After stirring for 30 min, the mixture was heated at 70° C. for 2 hours. The mixture was concentrated to dryness and purified by flash column chromatography to give the titled compound (21.5 g, crude) which was used for the next step directly. MS: M/e 219 (M+1)⁺.

### Step F: tert-butyl (2R,5S)-4-benzyl-2-ethyl-5-methylpiperazine-1-carboxylate

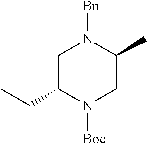 [SMILES: CC[C@@H]1CN(Cc2ccccc2)[C@@H](C)CN1C(=O)OC(C)(C)C]

To a solution of (2S,5R)-1-benzyl-5-ethyl-2-methylpiperazine (19.5 g, crude) in THF (150 mL) was added K₂CO₃ (24.5 g, 177 mmol) and H₂O (50 mL) at room temperature and followed by di-tert-butyl decarbonate (20.0 g, 91.7 mmol) in drops. The resulted mixture was stirred at room temperature for 16 hours. The mixture was diluted with of EtOAc (200 mL), washed with brine (100 mL×3), dried over Na₂SO₄, concentrated and purified by flash column chromatography to give the titled compound (23.8 g, 86% for 2 steps). MS: M/e 319 (M+1)⁺.

### Step G: tert-butyl (2R,5S)-2-ethyl-5-methylpiperazine-1-carboxylate

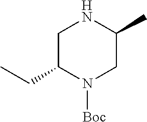 [SMILES: CC[C@@H]1CN[C@@H](C)CN1C(=O)OC(C)(C)C]

A mixture of tert-butyl (2R,5S)-4-benzyl-2-ethyl-5-methylpiperazine-1-carboxylate (23.8 g, 74.8 mmol), Pd/C (3.0 g), AcOH (2 mL) in MeOH (400 mL) was stirred at room temperature under H₂ for 16 hours. The mixture was filtered and the filtrate was concentrated. The resulted oil was treated with aqueous solution of NaOH (5M, 50 mL), extracted with CH₂Cl₂ (100 mL×3). The combined organic layers was washed with brine (100 mL×3), dried over Na₂SO₄, concentrated to dryness to give the titled compound (14.3 g, 84%). ¹H NMR (400 MHz, DMSO-d6) δ 3.42-3.23 (m, 1H), 3.04 (d, J=12.0 Hz, 1H), 2.64-2.46 (m, 3H), 2.15-2.05 (m, 1H), 1.99 (dd, J=12.8, 1.6 Hz, 1H), 1.41-1.24 (m, 1H), 1.16-1.01 (m, 1H), 0.97 (s, 9H), 0.60 (d, J=6.8 Hz, 3H), 0.36 (t, J=7.6 Hz, 3H). MS: M/e 229 (M+1)⁺.

### Step H: ethyl 8-((2S,5R)-4-(tert-butoxycarbonyl)-5-ethyl-2-methylpiperazin-1-yl)-6-chloroimidazo[1,2-b]pyridazine-2-carboxylate

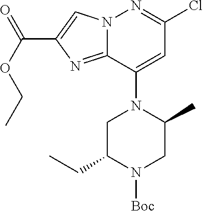 [SMILES: CCOC(=O)c1cn2nc(Cl)cc(N3C[C@@H](CC)N(C(=O)OC(C)(C)C)C[C@@H]3C)c2n1]

A mixture of ethyl 8-bromo-6-chloroimidazo[1,2-b]pyridazine-2-carboxylate (5.0 g, 16.6 mmol), tert-butyl (2R,5S)-2-ethyl-5-methylpiperazine-1-carboxylate (4.0 g, 17.5 mmol) and DIPEA (6.5 g, 50.5 mmol) in MeCN (40 mL) was stirred at 90° C. for 20 hours. The mixture was treated with saturated aqueous solution of NaHCO₃ (50 mL) and extracted with EtOAc (50 mL×3). The extracts were combined and washed with brine (50 mL×2), dried and concentrated. The resulted residue was purified by flash column chromatography to give the titled compound (6.61 g, 88%). MS: M/e 452 (M+1)⁺.

### Step I: 8-((2S,5R)-4-(tert-butoxycarbonyl)-5-ethyl-2-methylpiperazin-1-yl)-6-oxo-5,6-dihydroimidazo[1,2-b]pyridazine-2-carboxylic acid

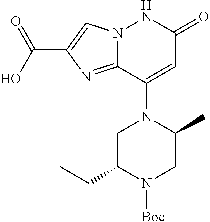 [SMILES: CC[C@@H]1CN(c2cc(=O)[nH]n3cc(C(=O)O)nc23)[C@@H](C)CN1C(=O)OC(C)(C)C]

A mixture of ethyl ethyl 8-((2S,5R)-4-(tert-butoxycarbonyl)-5-ethyl-2-methylpiperazin-1-yl)-6-chloroimidazo[1,2-b]pyridazine-2-carboxylate (5.0 g, 11.1 mmol), Pd2(dba)3 (2.0 g, 2.2 mmol), t-BuXPhos (1.9 g, 4.4 mmol) and KOH (3M, 9.0 mL) in Dioxane (90 mL) was stirred at 100° C. for 2 hours. Another KOH solution (30 mL, 3M) was added and the mixture was stirred at 50° C. for 1 hour. The mixture was extracted with DCM (50 mL×3). The aqueous layer was acidified by citric acid to pH<5 and extracted with DCM/MeOH (5/1, 50 mL×5). The combined organic layers was washed with brine (100 mL×2), dried, concentrated to give the titled compound (5.5 g, crude). MS: M/e 406 (M+1)⁺.

### Step J: methyl 8-((2S,5R)-4-(tert-butoxycarbonyl)-5-ethyl-2-methylpiperazin-1-yl)-5-methyl-6-oxo-5,6-dihydroimidazo[1,2-b]pyridazine-2-carboxylate

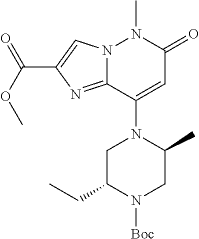 [SMILES: CC[C@@H]1CN(c2cc(=O)n(C)n3cc(C(=O)OC)nc23)[C@@H](C)CN1C(=O)OC(C)(C)C]

A mixture of 8-((2S,5R)-4-(tert-butoxycarbonyl)-5-ethyl-2-methylpiperazin-1-yl)-6-oxo-5,6-dihydroimidazo[1,2-b]pyridazine-2-carboxylic acid (5.5 g, crude), iodomethane (9.5 g, 66.9 mmol) and Cs₂CO₃ (15.0 g, 46.1 mmol) in Dioxane (60 mL) was stirred at 70° C. for 16 hours. The mixture was diluted with EtOAc (100 mL), and the suspension was filtered. The filtrate was concentrated and purified by flash column chromatography to give the titled compound (1.5 g, 27% for 2 steps). MS: M/e 434 (M+1)⁺.

### Step K: tert-butyl (2R,5S)-2-ethyl-4-(2-(hydroxymethyl)-5-methyl-6-oxo-5,6-dihydroimidazo[1,2-b]pyridazin-8-yl)-5-methylpiperazine-1-carboxylate

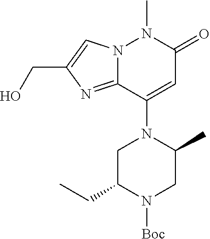 [SMILES: CC[C@@H]1CN(c2cc(=O)n(C)n3cc(CO)nc23)[C@@H](C)CN1C(=O)OC(C)(C)C]

To a stirred solution of methyl 8-((2S,5R)-4-(tert-butoxycarbonyl)-5-ethyl-2-methylpiperazin-1-yl)-5-methyl-6-oxo-5,6-dihydroimidazo[1,2-b]pyridazine-2-carboxylate (1.4 g, 3.23 mmol) in a mixed solvent THF/MeOH (1:3, 20 mL) was added NaBH₄ (1.0 g, 26.3 mmol) in portions at room temperature. The resulted mixture was stirred at 60° C. for 16 hours. Another 4.0 g of NaBH₄ was added within 47 hours (1.0 g of NaBH₄ at 60° C. for 5 hours, 1.0 g at 40° C. for 40 hours, and 2.0 g at 60° C. for 2 hours). All the start material consumed. The mixture was diluted with EtOAc (20 mL), and the suspension was filtered through a celite pad to remove the solid. The filtrate was concentrated and the residue was treated with of H₂O (20 mL), extracted with EtOAc (20 mL×3). The combined organic layers was washed with brine (20 mL×3), dried over Na₂SO₄, concentrated. The resulted crude product was purified by flash column chromatography to give the titled compound (710 mg, 54%). MS: M/e 406 (M+1)⁺.

### Step L: tert-butyl (2R,5S)-4-(2-(chloromethyl)-5-methyl-6-oxo-5,6-dihydroimidazo[1,2-b]pyridazin-8-yl)-2-ethyl-5-methylpiperazine-1-carboxylate

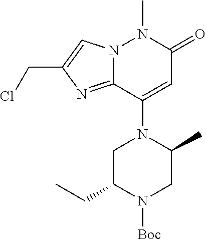 [SMILES: CC[C@@H]1CN(c2cc(=O)n(C)n3cc(CCl)nc23)[C@@H](C)CN1C(=O)OC(C)(C)C]

To a stirred solution of tert-butyl (2R,5S)-2-ethyl-4-(2-(hydroxymethyl)-5-methyl-6-oxo-5,6-dihydroimidazo[1,2-b]pyridazin-8-yl)-5-methylpiperazine-1-carboxylate (700 mg, 1.72 mmol) in CH₂Cl₂ (5 mL) was added SOCl₂ (415 mg, 3.5 mmol). The resulting mixture was stirred at room temperature for 2 hours. The mixture was quenched with saturated solution of NaHCO₃ (5 mL), extracted with DCM (5 ml×3). The combined organic layers was washed with brine (5 mL×2), dried over Na₂SO₄, and concentrated to dryness to give the titled compound (700 mg, 95%). MS: M/e 424 (M+1)⁺.

### Step M: tert-butyl (2R,5S)-4-(2-(cyanomethyl)-5-methyl-6-oxo-5,6-dihydroimidazo[1,2-b]pyridazin-8-yl)-2-ethyl-5-methylpiperazine-1-carboxylate

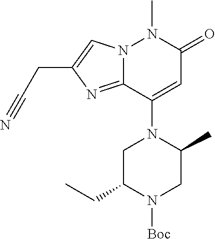 [SMILES: CC[C@@H]1CN(c2cc(=O)n(C)n3cc(CC#N)nc23)[C@@H](C)CN1C(=O)OC(C)(C)C]

A mixture of tert-butyl (2R,5S)-4-(2-(chloromethyl)-5-methyl-6-oxo-5,6-dihydroimidazo[1,2-b]pyridazin-8-yl)-2-ethyl-5-methylpiperazine-1-carboxylate (600 mg, 1.42 mmol), TMSCN (281 mg, 2.84 mmol) and Cs₂CO₃ (1.39 g, 4.26 mmol) in MeCN (10 mL) was stirred at 70° C. for 16 hours. The mixture was diluted with EtOAc (20 mL), filtered and the filtrate was washed with brine (10 mL×3), dried over Na₂SO₄, and concentrated to dryness. The resulting residue was purified by flash column chromatography to give the titled compound (320 mg, 54%). MS: M/e 415 (M+1)⁺.

### Step N: 2-(8-((2S,5R)-5-ethyl-2-methylpiperazin-1-yl)-5-methyl-6-oxo-5,6-dihydroimidazo[1,2-b]pyridazin-2-yl)acetonitrile

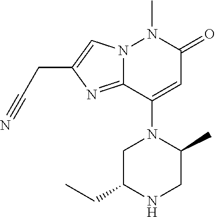 [SMILES: CC[C@@H]1CN(c2cc(=O)n(C)n3cc(CC#N)nc23)[C@@H](C)CN1]

To a stirred solution of tert-butyl (2R,5S)-4-(2-(cyanomethyl)-5-methyl-6-oxo-5,6-dihydroimidazo[1,2-b]pyridazin-8-yl)-2-ethyl-5-methylpiperazine-1-carboxylate (320 mg, 0.77 mmol) in CH₂Cl₂ (5 mL) was added TFA (1 mL). The resulted mixture was stirred at room temperature for 3 hours. The mixture was concentrated and the resulted residue was treated with saturated aqueous solution of NaHCO₃ to pH>8. The mixture was extracted with DCM (5 mL×3). The combined organic layers was washed with brine (5 mL×2), dried over Na₂SO₄ and concentrated to give the titled compound (210 mg, 86%). MS: M/e 315 (M+1)⁺.

### Step 0: 2-(8-((2S,5R)-5-ethyl-2-methyl-4-(1-(quinoxalin-6-yl)ethyl)piperazin-1-yl)-5-methyl-6-oxo-5,6-dihydroimidazo[1,2-b]pyridazin-2-yl acetonitrile

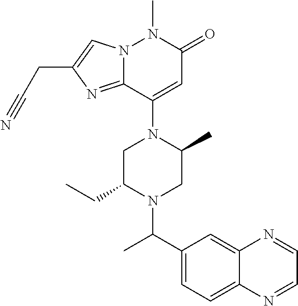 [SMILES: CC[C@@H]1CN(c2cc(=O)n(C)n3cc(CC#N)nc23)[C@@H](C)CN1C(C)c1ccc2nccnc2c1]

A mixture of 2-(8-((2S,5R)-5-ethyl-2-methylpiperazin-1-yl)-5-methyl-6-oxo-5,6-dihydroimidazo[1,2-b]pyridazin-2-yl)acetonitrile (100 mg, 0.32 mmol), 1-(quinoxalin-6-yl)ethan-1-ol (140 mg, 0.8 mmol), (cyanomethyl)trimethylphosphonium iodide (311 mg, 1.28 mmol) and DIPEA (250 mg, 1.94 mmol) in MeCN (1 mL) was stirred at 100° C. for 16 hours. The resulted mixture was diluted with EtOAc (5 mL), washed with brine (2 mL×3), dried and concentrated. The resulting residue was purified by flash column chromatography to give the titled Compound A13 which was separated into Compound A13a (5 mg) and Compound A13b (5 mg) by Prep-HPLC (Method B).

Compound A13a (the earlier peak): ¹H NMR (400 MHz, CDCl₃) δ 9.02-8.77 (m, 2H), 8.14-7.83 (m, 2H), 7.40-7.29 (m, 1H), 5.74-5.56 (m, 1H), 4.32-3.94 (m, 1H), 3.78-3.70 (m, 5H), 3.33-3.21 (m, 1H), 3.15-2.75 (m, 2H), 2.54-2.06 (m, 1H), 1.99-1.84 (m, 1H), 1.65-1.52 (m, 5H), 1.42 (d, J=5.2 Hz, 3H), 0.77-0.45 (m, 3H). MS: M/e 471 (M+1)⁺.

Compound A13b (the later peak): ¹H NMR (400 MHz, CDCl₃) δ 9.03-8.78 (m, 2H), 8.42-7.84 (m, 3H), 7.43-7.30 (m, 1H), 5.76-5.58 (m, 1H), 4.02-3.81 (m, 1H), 3.80-3.75 (m, 2H), 3.72 (s, 3H), 3.62-3.39 (m, 2H), 3.28-3.09 (m, 1H), 2.89-2.72 (m, 1H), 2.31-2.05 (m, 2H), 1.95-1.69 (m, 1H), 1.60-1.55 (m, 5H), 1.46-1.26 (m, 3H), 1.21-1.00 (m, 3H). MS: M/e 471 (M+1)⁺.

Compound A14: 2-(8-((2S,5R)-2,5-diethyl-4-(1-(quinoxalin-6-yl)ethyl)piperazin-1-yl)-5-methyl-6-oxo-5,6-dihydroimidazo[1,2-b]pyridazin-2-yl)acetonitrile

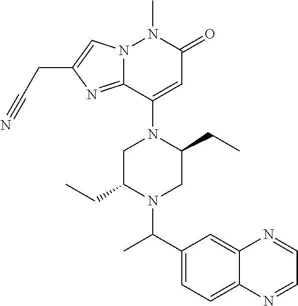 [SMILES: CC[C@H]1CN(C(C)c2ccc3nccnc3c2)[C@H](CC)CN1c1cc(=O)n(C)n2cc(CC#N)nc12]

### Step A: tert-butyl (2R,5S)-4-(2-(cyanomethyl)-5-methyl-6-oxo-5,6-dihydroimidazo[1,2-b]pyridazin-8-yl)-2,5-diethylpiperazine-1-carboxylate

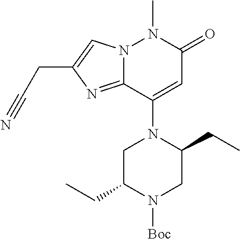 [SMILES: CC[C@@H]1CN(c2cc(=O)n(C)n3cc(CC#N)nc23)[C@@H](CC)CN1C(=O)OC(C)(C)C]

A mixture of tert-butyl (2R,5S)-4-(2-(chloromethyl)-5-methyl-6-oxo-5,6-dihydroimidazo[1,2-b]pyridazin-8-yl)-2,5-diethylpiperazine-1-carboxylate (150 mg, 0.35 mmol), TMSCN (69 mg, 0.69 mmol) and Cs₂CO₃ (300 mg, 0.90 mmol) in MeCN (4 mL) was stirred at 70° C. for 24 hours. The mixture was diluted with EtOAc (10 mL), washed with brine (5 mL×3), dried over Na₂SO₄, concentrated and purified by flash column chromatography to give the titled compound (92 mg, 71%). MS: M/e 429 (M+1)⁺.

### Step B: 2-(8-((2S,5R)-2,5-diethylpiperazin-1-yl)-5-methyl-6-oxo-5,6-dihydroimidazo[1,2-b]pyridazin-2-yl)acetonitrile

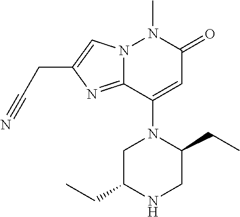 [SMILES: CC[C@@H]1CN(c2cc(=O)n(C)n3cc(CC#N)nc23)[C@@H](CC)CN1]

To a stirred solution of tert-butyl (2R,5S)-4-(2-(cyanomethyl)-5-methyl-6-oxo-5,6-dihydroimidazo[1,2-b]pyridazin-8-yl)-2,5-diethylpiperazine-1-carboxylate (92 mg, 0.21 mmol) in CH₂Cl₂ (5 mL) was added TFA (1 mL). The resulted mixture was stirred at room temperature for 3 hours. The mixture was concentrated and the resulted residue was treated with saturated aqueous solution of NaHCO₃ to pH>8. The mixture was extracted with DCM (5 mL×3). The combined organic layers was washed with brine (5 mL×2), dried over Na₂SO₄ and concentrated to give the titled compound (40 mg, 58%). MS: M/e 329 (M+1)⁺.

### Step C: 2-(8-((2S,5R)-2,5-diethyl-4-(1-(quinoxalin-6-yl)ethyl)piperazin-1-yl)-5-methyl-6-oxo-5,6-dihydroimidazo[1,2-b]pyridazin-2-yl)acetonitrile

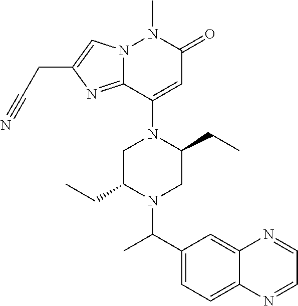 [SMILES: CC[C@H]1CN(C(C)c2ccc3nccnc3c2)[C@H](CC)CN1c1cc(=O)n(C)n2cc(CC#N)nc12]

A mixture of 2-(8-((2S,5R)-2,5-diethylpiperazin-1-yl)-5-methyl-6-oxo-5,6-dihydroimidazo[1,2-b]pyridazin-2-yl)acetonitrile (40 mg, 0.12 mmol), 1-(quinoxalin-6-yl)ethan-1-ol (55 mg, 0.3 mmol), (cyanomethyl)trimethylphosphonium iodide (100 mg, 0.41 mmol) and DIPEA (150 mg, 1.16 mmol) in MeCN (1 mL) was stirred at 100° C. for 16 hours. The resulted mixture was diluted with EtOAc (5 mL), washed with brine (2 mL×3), dried and concentrated. The resulting residue was purified by flash column chromatography to give the titled Compound A14 which was separated into Compound A14a (12 mg) and Compound A14b (19 mg) by Prep-HPLC (Method B).

Compound A14a (the earlier peak): ¹H NMR (400 MHz, CD₃OD) δ 8.96-8.81 (m, 2H), 8.12 (d, J=8.4 Hz, 1H), 8.05 (s, 1H), 8.03-7.97 (m, 1H), 7.78 (s, 1H), 5.62 (s, 1H), 4.07 (q, J=6.4 Hz, 1H), 3.92 (s, 2H), 3.73 (s, 3H), 3.38-3.31 (m, 3H), 3.08 (d, J=12.0 Hz, 1H), 2.96 (dd, J=12.0, 3.2 Hz, 1H), 2.43 (d, J=8.8 Hz, 1H), 2.24-2.05 (m, 1H), 2.00-1.79 (m, 1H), 1.70-1.51 (m, 2H), 1.46 (d, J=6.4 Hz, 3H), 0.97 (t, J=7.2 Hz, 3H), 0.68 (t, J=7.2 Hz, 3H) ppm. MS: M/e 485 (M+1)⁺.

Compound A14b (the later peak): ¹H NMR (400 MHz, CD₃OD) δ 8.87 (dd, J=6.8, 1.6 Hz, 2H), 8.12-8.06 (m, 2H), 8.02 (dd, J=8.8, 1.2 Hz, 1H), 7.79 (s, 1H), 5.60 (s, 1H), 3.98-3.85 (m, 3H), 3.73 (s, 3H), 3.56 (d, J=13.2 Hz, 1H), 3.30-3.29 (m, 2H), 3.23 (d, J=9.6 Hz, 1H), 2.83-2.70 (m, 1H), 2.37 (d, J=12.4 Hz, 1H), 2.08-1.90 (m, 1H), 1.80-1.53 (m, 3H), 1.43 (d, J=6.4 Hz, 3H), 1.07 (t, J=7.2 Hz, 3H), 0.56 (t, J=7.2 Hz, 3H) ppm. MS: M/e 485 (M+1)⁺.

Compound A15: 2-(8-((2S,5R)-2,5-diethyl-4-(1-(quinoxalin-6-yl)ethyl)piperazin-1-yl)-6-oxo-5,6-dihydroimidazo[1,2-b]pyridazin-2-yl)acetonitrile

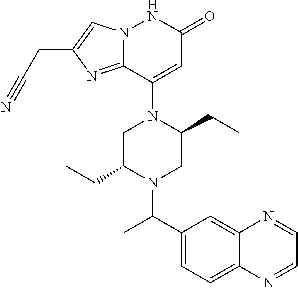 [SMILES: CC[C@H]1CN(C(C)c2ccc3nccnc3c2)[C@H](CC)CN1c1cc(=O)[nH]n2cc(CC#N)nc12]

### Step A: Ethyl8-((2S,5R)-4-benzyl-2,5-diethylpiperazin-1-yl)-6-chloroimidazo[1,2-b]pyridazine-2-carboxylate

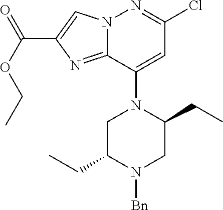 [SMILES: CCOC(=O)c1cn2nc(Cl)cc(N3C[C@@H](CC)N(Cc4ccccc4)C[C@@H]3CC)c2n1]

To a solution of methyl 8-bromo-6-chloroimidazo[1,2-b]pyridazine-2-carboxylate (310 mg, 1 mmol) and DIPEA (258 mg, 2 mmol) in CH₃CN (6 mL) was added (2R,5S)-1-benzyl-2,5-diethylpiperazine (300 mg, 1 mmol). The reaction was stirred at 70° C. for 16 hours. Then the mixture was cooled to room temperature, diluted with water, extracted with EA (60 mL×3), washed with brine, dried over Na₂SO₄, filtered, and concentrated. The resulting residue was purified by flash column chromatography to give the titled compound (220 mg, 48%). MS: M/e 456 (M+1)⁺.

### Step B: 8-((2S,5R)-4-benzyl-2,5-diethylpiperazin-1-yl)-6-(benzyloxy)imidazo[1,2-b]pyridazine-2-carboxylic acid

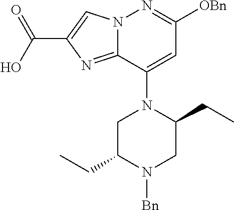 [SMILES: CC[C@@H]1CN(c2cc(OCc3ccccc3)nn3cc(C(=O)O)nc23)[C@@H](CC)CN1Cc1ccccc1]

To a solution of BnOH (432 mg, 4 mmol) in DMSO (300 mL) was added 60% NaH (320 mg, 8 mmol). The mixture was stirred at room temperature for 30 min. Then a solution of Ethyl8-((2S,5R)-4-benzyl-2,5-diethylpiperazin-1-yl)-6-chloroimidazo[1,2-b]pyridazine-2-carboxylate (910 mg, 2 mmol) in DMSO (2 mL) was added and heated at 80° C. overnight. The mixture was cooled to room temperature, diluted with water, washed with EA. The water layer was collected, treated with 2N HCl to pH=3, extracted with EA (60 mL×3), washed with brine, dried over Na₂SO₄, filtered, and concentrated to give the titled compound. (1.3 g, crude). MS: M/e 500 (M+1)⁺.

### Step C: ethyl 8-((2S,5R)-4-benzyl-2,5-diethylpiperazin-1-yl)-6-(benzyloxy)imidazo[1,2-b]pyridazine-2-carboxylate

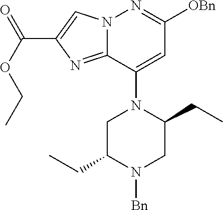 [SMILES: CCOC(=O)c1cn2nc(OCc3ccccc3)cc(N3C[C@@H](CC)N(Cc4ccccc4)C[C@@H]3CC)c2n1]

To a mixture of 8-((2S,5R)-4-benzyl-2,5-diethylpiperazin-1-yl)-6-(benzyloxy)imidazo[1,2-b]pyridazine-2-carboxylic acid (1.35 g, crude) in 1,4-dioxane (8 mL) was added Cs₂CO₃ (1.3 g 4 mmol) and iodoethane (620 mg, 4 mmol). The reaction was heated at 80° C. overnight. The mixture was cooled to room temperature, diluted with water, extracted with EA (60 mL×2), washed with brine, dried over Na₂SO₄, filtered, and concentrated. The residue was purified by flash column chromatography (EA:PE=1:3) to give the titled compound (0.57 g, crude). MS: M/e 528 (M+1)⁺.

### Step D: (8-((2S,5R)-4-benzyl-2,5-diethylpiperazin-1-yl)-6-(benzyloxy)imidazo[1,2-b]pyridazin-2-yl)methanol

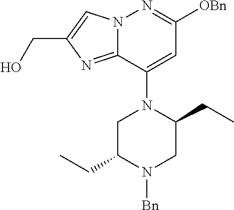 [SMILES: CC[C@@H]1CN(c2cc(OCc3ccccc3)nn3cc(CO)nc23)[C@@H](CC)CN1Cc1ccccc1]

To a mixture of ethyl 8-((2S,5R)-4-benzyl-2,5-diethylpiperazin-1-yl)-6-(benzyloxy)imidazo[1,2-b]pyridazine-2-carboxylate (570 mg, crude) in THF (10 mL) was added a solution of LiAlH₄ in THF (2.2 mL, 2.2 mmol) dropwise. The reaction was stirred at rt for 2 hours. The reaction was quenched with water, extracted with EA (60 mL×3), washed with brine, dried over Na₂SO₄, filtered, and concentrated. The resulting residue was purified by flash column chromatography (EA:PE=1:3) to give the titled compound (0.48 g, crude). MS: M/e 486 (M+1)⁺.

### Step E: 2-(8-((2S,5R)-4-benzyl-2,5-diethylpiperazin-1-yl)-6-(benzyloxy)imidazo[1,2-b]pyridazin-2-yl)acetonitrile

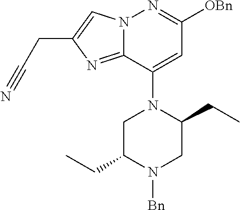 [SMILES: CC[C@@H]1CN(c2cc(OCc3ccccc3)nn3cc(CC#N)nc23)[C@@H](CC)CN1Cc1ccccc1]

To a mixture of (8-((2S,5R)-4-benzyl-2,5-diethylpiperazin-1-yl)-6-(benzyloxy)imidazo[1,2-b]pyridazin-2-yl)methanol (480 mg, crude) in DCM (6 mL) was added SOCl₂ (104 mg, 0.88 mmol). The reaction was stirred at room temperature for 0.5 h, quenched with water, extracted with DCM (60 mL×2), washed with brine, dried over Na₂SO₄, filtered, and concentrated. The residue was dissolved in CH₃CN (10 mL), Cs₂CO₃ (858 mg, 2.64 mmol) and TMSCN (350 mg, 3.52 mmol) was added. The reaction was heated at 70° C. overnight under N₂. The mixture was cooled to room temperature, diluted with water, extracted with EA (60 mL×2), washed with brine, dried over Na₂SO₄, filtered, and concentrated. The resulting residue was purified by flash column chromatography (EA:PE=1:3) to give the titled compound (360 mg, crude). MS: M/e 495 (M+1)⁺.

### Step F: 2-(8-((2S,5R)-2,5-diethylpiperazin-1-yl)-6-hydroxyimidazo[1,2-b]pyridazin-2-yl)acetonitrile

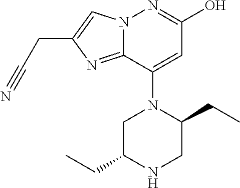 [SMILES: CC[C@@H]1CN(c2cc(O)nn3cc(CC#N)nc23)[C@@H](CC)CN1]

To a mixture of 2-(8-((2S,5R)-4-benzyl-2,5-diethylpiperazin-1-yl)-6-(benzyloxy)imidazo[1,2-b]pyridazin-2-yl)acetonitrile (100 mg, crude) in MeOH (5 mL) was added 10% Pd/C (10 mg) and a drop of TFA. The reaction was stirred at room temperature for 4 hours under H2 (balloon). The mixture was filtered through a pad of Celite, washed with MeOH. The filtrate was collected, treated with 7M NH3 in MeOH, and reconcentrated. The resulting residue was purified by prep-TLC (DCM:MeOH=10:1) to give the titled compound (35 mg, crude).

### Step G: 2-(8-((2S,5R)-2,5-diethyl-4-(1-(quinoxalin-6-yl)ethyl)piperazin-1-yl)-6-oxo-5,6-dihydroimidazo[1,2-b]pyridazin-2-yl)acetonitrile

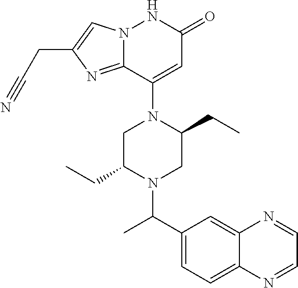 [SMILES: CC[C@H]1CN(C(C)c2ccc3nccnc3c2)[C@H](CC)CN1c1cc(=O)[nH]n2cc(CC#N)nc12]

To a solution of 2-(8-((2S,5R)-2,5-diethylpiperazin-1-yl)-6-hydroxyimidazo[1,2-b]pyridazin-2-yl)acetonitrile (35 mg, 0.1 mmol), 1-(quinoxalin-6-yl)ethan-1-ol (35 mg, 0.2 mmol) and (cyanomethyl)trimethylphosphonium iodide (48 mg, 0.2 mmol) in CH₃CN (3 mL) was added DIPEA (38 mg, 0.3 mmol). The mixture was sealed in a bottle and heated at 105° C. for 16 hours. Another portion of (cyanomethyl)trimethylphosphonium iodide (48 mg, 0.2 mmol) was added and heated at 105° C. for 18 hours. Then the mixture was cooled to room temperature, diluted with water, extracted with EA (60 mL), washed with brine, dried over Na₂SO₄, filtered, and concentrated. The resulting residue was purified by prep-TLC (DCM:MeOH=15:1) and prep-HPLC (Method A) to give the titled compound (1 mg). ¹HNMR (400 MHz, CD₃OD) δ 8.92-8.82 (m, 2H), 8.15-7.90 (m, 3H), 7.55 (s, 1H), 5.72 (s, 1H), 4.12-4.02 (m, 0.5H), 3.97-3.86 (m, 0.5H), 3.89 (s, 2H), 3.62-3.44 (m, 1H), 3.26-3.15 (m, 1H), 3.12-2.94 (m, 2H), 2.82-2.74 (m, 1H), 2.47-2.30 (m, 1H), 2.22-2.08 (m, 0.5H), 2.00-1.90 (m, 0.5H), 1.87-1.56 (m, 3H), 1.49-1.40 (m, 3H), 1.05 (t, J=7.6 Hz, 1.5H), 0.94 (t, J=7.6 Hz, 1.5H), 0.66 (t, J=7.6 Hz, 1.5H), 0.54 (t, J=7.6 Hz, 1.5H) ppm. MS: M/e 471 (M+1)⁺.

### Compound A16: 8-((2S,5R)-5-ethyl-4-(1-(2-fluoro-4-(trifluoromethyl)phenyl)ethyl)-2-methylpiperazin-1-yl)-2-(methoxymethyl)-5-methylimidazo[1,2-b]pyridazin-6(5H)-one

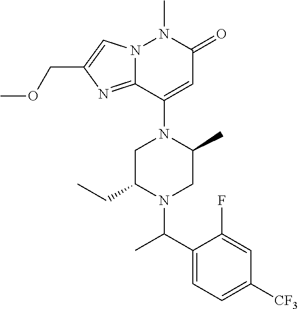 [SMILES: CC[C@@H]1CN(c2cc(=O)n(C)n3cc(COC)nc23)[C@@H](C)CN1C(C)c1ccc(C(F)(F)F)cc1F]

### Step A: tert-butyl (2R,5S)-2-ethyl-4-(2-(methoxymethyl)-5-methyl-6-oxo-5,6-dihydroimidazo[1,2-b]pyridazin-8-yl)-5-methylpiperazine-1-carboxylate

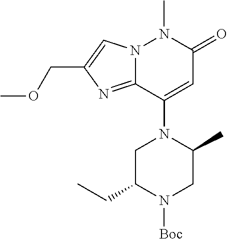

tert-butyl (2R,5S)-4-(2-(chloromethyl)-5-methyl-6-oxo-5,6-dihydroimidazo[1,2-b]pyridazin-8-yl)-2-ethyl-5-methylpiperazine-1-carboxylate (100 mg, 0.23 mmol) was added into a solution of CH₃ONa in MeOH (5.4 M, 1 mL) and the mixture was stirred at 70° C. for 1 hour. The mixture was treated with H₂O (5 mL), extracted with EtOAc (5 mL×3). The combined organic layers was washed with brine (10 mL×2), dried over Na₂SO₄ and concentrated. The resulting residue was purified by flash column chromatography to give the titled compound (75 mg, 78%). MS: M/e 420 (M+1)⁺.

### Step B: 8-((2S,5R)-5-ethyl-2-methylpiperazin-1-yl)-2-(methoxymethyl)-5-methylimidazo[1,2-b]pyridazin-6(5H)-one

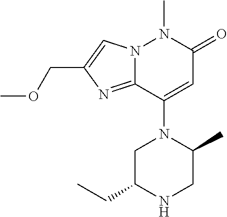 [SMILES: CC[C@@H]1CN(c2cc(=O)n(C)n3cc(COC)nc23)[C@@H](C)CN1]

To a stirred solution of tert-butyl (2R,5S)-2-ethyl-4-(2-(methoxymethyl)-5-methyl-6-oxo-5,6-dihydroimidazo[1,2-b]pyridazin-8-yl)-5-methylpiperazine-1-carboxylate (75 mg, 0.18 mmol) in CH₂Cl₂ (2 mL) was added TFA (1 mL). The resulted mixture was stirred at room temperature for 3 hours. The mixture was concentrated and the resulted residue was treated with saturated aqueous solution of NaHCO₃ to pH>8. The mixture was extracted with DCM (5 mL×3). The combined organic layers was washed with brine (5 mL×2), dried over Na₂SO₄ and concentrated to give the titled compound (52 mg, 90%). MS: M/e 320 (M+1)⁺.

### Step C: 8-((2S,5R)-5-ethyl-4-(1-(2-fluoro-4-(trifluoromethyl)phenyl)ethyl)-2-methylpiperazin-1-yl)-2-(methoxymethyl)-5-methylimidazo[1,2-b]pyridazin-6(5H)-one

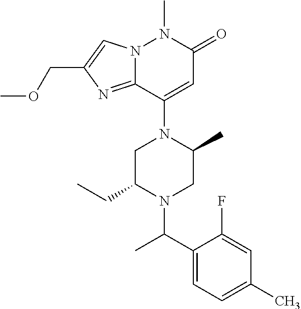

A mixture of 8-((2S,5R)-5-ethyl-2-methylpiperazin-1-yl)-2-(methoxymethyl)-5-methylimidazo[1,2-b]pyridazin-6(5H)-one (52 mg, 0.16 mmol), 1-(2-fluoro-4-(trifluoromethyl)phenyl)ethan-1-ol (85 mg, 0.40 mmol), (cyanomethyl)trimethylphosphonium iodide (155 mg, 0.64 mmol) and DIPEA (129 mg, 1.0 mmol) in MeCN (1 mL) was stirred at 100° C. for 16 hours. The resulted mixture was diluted with EtOAc (5 mL), washed with brine (2 mL×3), dried and concentrated. The resulting residue was purified by flash column chromatography to give the titled Compound A16 which was separated into Compound A16a (1 mg) and Compound A16b (2 mg) by Prep-HPLC (Method B).

Compound A16a (the earlier peak): ¹H NMR (400 MHz, CDCl₃) δ 7.74-7.58 (m, 1H), 7.47-7.39 (m, 1H), 7.38-7.28 (m, 2H), 5.70-5.54 (m, 1H), 4.51-4.41 (m, 2H), 4.31-4.15 (m, 1H), 3.72 (s, 3H), 3.43 (s, 3H), 3.29-3.10 (m, 1H), 3.04-2.92 (m, 1H), 2.81-2.66 (m, 1H), 2.47-2.32 (m, 1H), 2.06-1.99 (m, 1H), 1.91-1.81 (m, 1H), 1.62-1.53 (m, 4H), 1.37-1.30 (m, 4H), 0.82-0.55 (m, 3H). MS: M/e 510 (M+1)⁺.

Compound A16b (the later peak): ¹H NMR (400 MHz, CDCl₃) δ 7.86-7.61 (m, 1H), 7.47-7.37 (m, 1H), 7.37-7.27 (m, 2H), 5.75-5.57 (m, 1H), 4.52-4.42 (m, 2H), 4.10-3.94 (m, 1H), 3.72 (s, 3H), 3.49-3.40 (m, 4H), 3.16-3.00 (m, 1H), 2.86-2.69 (m, 1H), 2.19-2.11 (m, 1H), 2.04-1.97 (m, 1H), 1.89-1.67 (m, 1H), 1.62-1.51 (m, 5H), 1.33-1.26 (m, 3H), 1.21-0.98 (m, 3H). MS: M/e 510 (M+1)⁺.

### Compound A17: 2-(8-((2S,5R)-5-ethyl-4-(1-(2-fluoro-4-(trifluoromethyl)phenyl)ethyl)-2-methylpiperazin-1-yl)-5-methyl-6-oxo-5,6-dihydroimidazo[1,2-b]pyridazin-2-yl)acetonitrile

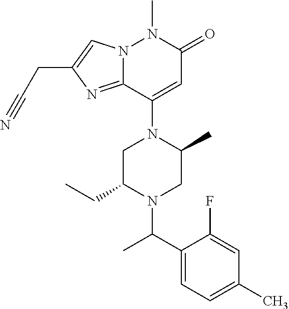 [SMILES: CC[C@@H]1CN(c2cc(=O)n(C)n3cc(CC#N)nc23)[C@@H](C)CN1C(C)c1ccc(C)cc1F]

A mixture of 2-(8-((2S,5R)-5-ethyl-2-methylpiperazin-1-yl)-5-methyl-6-oxo-5,6-dihydroimidazo[1,2-b]pyridazin-2-yl)acetonitrile (45 mg, 0.14 mmol), 1-(2-fluoro-4-(trifluoromethyl)phenyl)ethan-1-ol (65 mg, 0.31 mmol), (cyanomethyl)trimethylphosphonium iodide (100 mg, 0.41 mmol) and DIPEA (100 mg, 0.7 mmol) in MeCN (1 mL) was stirred at 100° C. for 16 hours. The resulted mixture was diluted with EtOAc (5 mL), washed with brine (2 mL×3), dried and concentrated. The resulting residue was purified by flash column chromatography to give the titled Compound A17 which was separated into Compound A17a (3 mg) and Compound A17b (4 mg) by Prep-HPLC (Method B).

Compound A17a (the earlier peak): ¹H NMR (400 MHz, CDCl₃) δ 7.73-7.61 (m, 1H), 7.47-7.40 (m, 1H), 7.40-7.28 (m, 2H), 5.80-5.58 (m, 1H), 4.30-4.16 (m, 1H), 3.80-3.68 (m, 5H), 3.28-3.12 (m, 1H), 3.04-2.88 (m, 1H), 2.83-2.67 (m, 1H), 2.48-2.28 (m, 1H), 2.15-1.95 (m, 1H), 1.94-1.78 (m, 1H), 1.63-1.48 (m, 4H), 1.39-1.30 (m, 4H), 0.85-0.53 (m, 3H). MS: M/e 505 (M+1)⁺.

Compound A17b (the later peak): ¹H NMR (400 MHz, CDCl₃) δ 7.83-7.62 (m, 1H), 7.48-7.26 (m, 3H), 5.77-5.58 (m, 1H), 4.15-3.93 (m, 1H), 3.81-3.68 (m, 5H), 3.51-3.35 (m, 1H), 3.27-3.02 (m, 1H), 2.89-2.68 (m, 1H), 2.25-1.95 (m, 2H), 1.92-1.66 (m, 1H), 1.60-1.52 (m, 4H), 1.36-1.22 (m, 4H), 1.22-0.96 (m, 3H). MS: M/e 505 (M+1)⁺.

### Compound A18: 2-(8-((2S,5R)-5-ethyl-4-(1-(4-fluoro-2-(trifluoromethyl)phenyl)ethyl)-2-methylpiperazin-1-yl)-5-methyl-6-oxo-5,6-dihydroimidazo[1,2-b]pyridazin-2-yl)acetonitrile

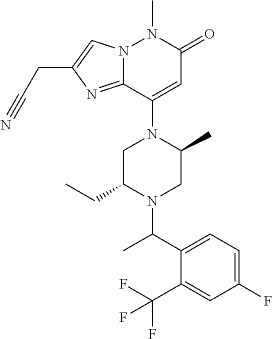 [SMILES: CC[C@@H]1CN(c2cc(=O)n(C)n3cc(CC#N)nc23)[C@@H](C)CN1C(C)c1ccc(F)cc1C(F)(F)F]

A mixture of 2-(8-((2S,5R)-5-ethyl-2-methylpiperazin-1-yl)-5-methyl-6-oxo-5,6-dihydroimidazo[1,2-b]pyridazin-2-yl)acetonitrile (44 mg, 0.14 mmol), 1-(4-fluoro-2-(trifluoromethyl)phenyl)ethan-1-ol (63 mg, 0.30 mmol), (cyanomethyl)trimethylphosphonium iodide (98 mg, 0.40 mmol) and DIPEA (100 mg, 0.7 mmol) in MeCN (1 mL) was stirred at 100° C. for 24 hours. The resulted mixture was diluted with EtOAc (5 mL), washed with brine (2 mL×3), dried and concentrated. The resulting residue was purified by flash column chromatography to give the crude product which was further purified by Prep-HPLC (Method A) to give the titled compound (6 mg, 9%). ¹H NMR (400 MHz, CD₃OD) δ 8.14-8.01 (m, 1H), 7.79 (s, 1H), 7.47-7.30 (m, 2H), 5.71-5.58 (m, 1H), 4.22-3.99 (m, 1H), 3.97-3.88 (m, 2H), 3.73 (s, 3H), 3.59-3.42 (m, 1H), 3.30-3.16 (m, 2H), 3.07-2.89 (m, 1H), 2.88-2.71 (m, 1H), 2.39-1.98 (m, 1H), 1.82-1.48 (m, 2H), 1.47-1.29 (m, 3H), 1.29-1.15 (m, 3H), 1.14-0.73 (m, 3H). MS: M/e 505 (M+1)⁺.

### Compound A19: 2-(8-((2S,5R)-4-(1-(3-(difluoromethyl)quinoxalin-6-yl)ethyl)-2,5-dimethylpiperazin-1-yl)-5-methyl-6-oxo-5,6-dihydroimidazo[1,2-b]pyridazin-2-yl)acetonitrile

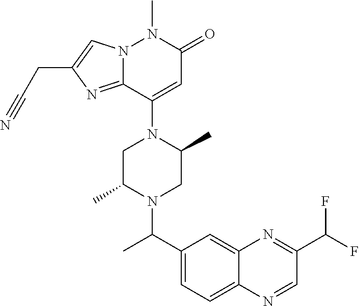 [SMILES: CC(c1ccc2ncc(C(F)F)nc2c1)N1C[C@H](C)N(c2cc(=O)n(C)n3cc(CC#N)nc23)C[C@H]1C]

A solution of 2-(8-((2S,5R)-2,5-dimethylpiperazin-1-yl)-5-methyl-6-oxo-5,6-dihydroimidazo[1,2-b]pyridazin-2-yl)acetonitrile (80 mg, 0.267 mmol), 1-(3-(difluoromethyl)quinoxalin-6-yl)ethan-1-ol (120 mg, 0.533 mmol), (cyanomethyl)trimethylphosphonium iodide (130 mg. 0.533 mmol) and DIPEA (103 mg, 0.801 mmol) in CH₃CN (2 ml). The mixture solution was degassed 3 times under N₂ atmosphere. Then the mixture solution was stirred at 105° C. for 24 hours. The reaction mixture was concentrated under reduced pressure. The crude product was purified by Prep-TLC (DCM:MeOH=15:1) to give the titled compound (55 mg, 41%). ¹H NMR (400 MHz, CD₃OD) δ 9.13 (d, J=4.3 Hz, 1H), 8.21-8.10 (m, 3H), 7.78 (s, 1H), 6.99 (td, J=54.5, 3.6 Hz, 1H), 5.62 (s, 1H), 4.68-4.52 (m, 1H), 4.00 (q, J=6.6 Hz, 0.5H), 3.93 (d, J=2.7 Hz, 2H), 3.85 (q, J=6.4 Hz, 0.5H), 3.73 (s, 3H), 3.72-3.64 (m, 1H), 3.46 (dd, J=12.9, 3.2 Hz, 0.5H), 3.30-3.23 (m, 0.5H), 3.09 (dd, J=11.7, 3.8 Hz, 0.5H), 2.97-2.83 (m, 2H), 2.20 (d, J=12.1 Hz, 0.5H), 1.47-1.39 (m, 4H), 1.23 (t, J=7.3 Hz, 3.5H), 1.07 (d, J=6.5 Hz, 1.5H) ppm. MS: M/e 507 (M+1)⁺.

### Compound A20: 2-(8-((2S,5R)-4-(1-(3-methoxyquinoxalin-6-yl)ethyl)-2,5-dimethylpiperazin-1-yl)-5-methyl-6-oxo-5,6-dihydroimidazo[1,2-b]pyridazin-2-yl)acetonitrile

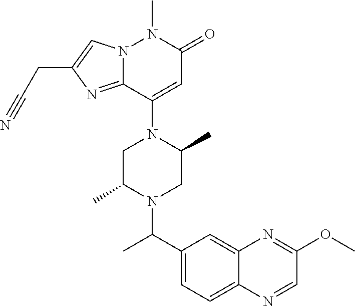 [SMILES: COc1cnc2ccc(C(C)N3C[C@H](C)N(c4cc(=O)n(C)n5cc(CC#N)nc45)C[C@H]3C)cc2n1]

### Step A: 7-bromo-2-methoxyquinoxaline

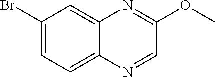

To a solution of 7-bromo-2-chloroquinoxaline (12 g, 49.3 mmol) in CH₃OH (150 mL) was added K₂CO₃ (13.6 g, 98.6 mmol). The reaction mixture was refluxed for 2 hours. After filtered, the organic layer was concentrated to give the titled compound (9.2 g, 79%). MS: M/e 239 (M+1)⁺

### Step B: 1-(3-methoxyquinoxalin-6-yl)ethan-1-one

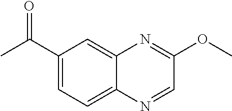

To a solution of 7-bromo-2-methoxyquinoxaline (4.0 g, 16.81 mmol), tributyl(1-ethoxyvinyl)stannane (12.14 g, 33.61 mmol) in toluene (70 mL) was added Pd(PPh₃)₂Cl₂ (2.36 g, 3.36 mmol). The reaction mixture was stirred at 90° C. under N₂ for 8 hours. The reaction mixture was quenched with saturated NaHCO₃ aq. (50 mL), extracted with EA (60 mL×2), combined organic layers, washed with brine (50 mL×2), dried and concentrated to dryness. The resulting oil was diluted with THF (50 mL). Then to the above solution was added HCl (6 mL, 4 M in 1,4-dioxane) in drops and the mixture was stirred at room temperature for 1 hour. The reaction mixture was diluted with EA (50 mL), treated with saturated NaHCO₃ aq. to pH-8, washed with brine (30 mL×3), dried over Na₂SO₄ and concentrated to dryness. The resulting residue was purified by flash column chromatography (PE: EA=8:1) to give the titled compound (1.2 g, 35%). MS: M/e 203 (M+1)⁺

### Step C: 1-(3-methoxyquinoxalin-6-yl)ethan-1-ol

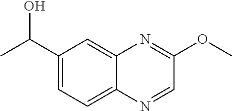

To a solution of 1-(3-methoxyquinoxalin-6-yl)ethan-1-one (800 mg, 3.94 mmol) in CH₃OH (15 mL) was added NaBH₄ (165 mg, 4.33 mmol). The reaction was stirred at room temperature for 1 hour. The reaction mixture was quenched with saturated NH4Cl, extracted with EA (35 mL×2), washed with brine, dried over Na₂SO₄, filtered, and concentrated to dryness. The resulting residue was purified by flash column chromatography (PE: EA=5:1) to give the titled compound (630 mg, 78%). MS: M/e 205 (M+1)⁺

### Step D: 2-(8-((2S,5R)-4-(1-(3-methoxyquinoxalin-6-yl)ethyl)-2,5-dimethylpiperazin-1-yl)-5-methyl-6-oxo-5,6-dihydroimidazo[1,2-b]pyridazin-2-yl)acetonitrile

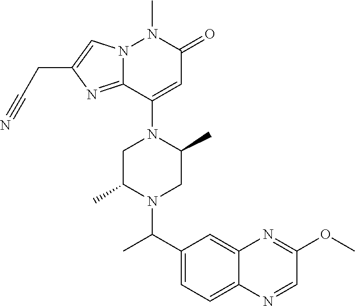 [SMILES: COc1cnc2ccc(C(C)N3C[C@H](C)N(c4cc(=O)n(C)n5cc(CC#N)nc45)C[C@H]3C)cc2n1]

A solution of 2-(8-((2S,5R)-2,5-dimethylpiperazin-1-yl)-5-methyl-6-oxo-5,6-dihydroimidazo[1,2-b]pyridazin-2-yl)acetonitrile (304 mg, 1.01 mmol), 1-(3-methoxyquinoxalin-6-yl)ethan-1-ol (310 mg, 1.52 mmol), (cyanomethyl)trimethylphosphonium iodide (491 mg. 2.02 mmol) and DIPEA (391 mg, 3.03 mmol) in CH₃CN (2 ml) was degassed 3 times under N₂ atmosphere. Then the mixture solution was stirred at 105° C. for 24 hours. The reaction mixture was concentrated under reduced pressure. The resulting crude product was purified by Prep-TLC (DCM:MeOH=15:1) to give the titled compound (255 mg, 52%). ¹H NMR (400 MHz, CD₃OD) δ 8.43 (d, J=5.0 Hz, 1H), 7.96 (dd, J=12.4, 8.5 Hz, 1H), 7.84 (d, J=6.2 Hz, 1H), 7.78 (s, 1H), 7.75 (d, J=8.5 Hz, 1H), 5.62 (s, 1H), 5.10-4.50 (m, 1H), 4.10 (d, J=5.5 Hz, 3H), 3.93 (d, J=2.4 Hz, 2H), 3.89-3.77 (m, 1H), 3.73 (s, 3H), 3.69-3.63 (m, 1H), 3.44-3.34 (m, 1H), 3.07 (dd, J=11.8, 3.9 Hz, 0.5H), 2.97-2.71 (m, 2H), 2.22 (d, J=12.2 Hz, 0.5H), 1.42 (dd, J=15.1, 6.5 Hz, 4.5H), 1.22 (dd, J=14.7, 6.5 Hz, 3H), 1.04 (d, J=6.5 Hz, 1.5H) ppm. MS: M/e 487 (M+1)⁺.

### Compound A21: 2-(8-((2S,5R)-4-(1-(3-chloroquinoxalin-6-yl)ethyl)-2,5-dimethylpiperazin-1-yl)-5-methyl-6-oxo-5,6-dihydroimidazo[1,2-b]pyridazin-2-yl)acetonitrile

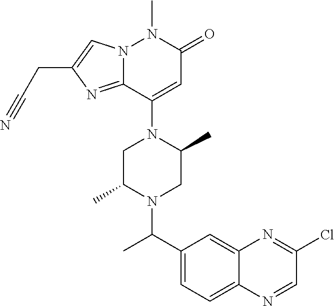 [SMILES: CC(c1ccc2ncc(Cl)nc2c1)N1C[C@H](C)N(c2cc(=O)n(C)n3cc(CC#N)nc23)C[C@H]1C]

### Step A: 2-(8-((2S,5R)-4-(1-(3-hydroxyquinoxalin-6-yl)ethyl)-2,5-dimethylpiperazin-1-yl)-5-methyl-6-oxo-5,6-dihydroimidazo[1,2-b]pyridazin-2-yl)acetonitrile

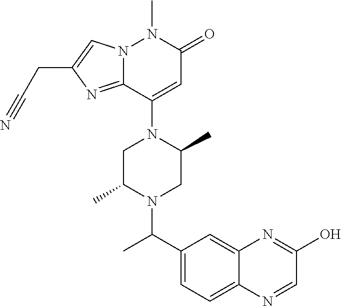 [SMILES: CC(c1ccc2ncc(O)nc2c1)N1C[C@H](C)N(c2cc(=O)n(C)n3cc(CC#N)nc23)C[C@H]1C]

To a solution of 2-(8-((2S,5R)-4-(1-(3-methoxyquinoxalin-6-yl)ethyl)-2,5-dimethylpiperazin-1-yl)-5-methyl-6-oxo-5,6-dihydroimidazo[1,2-b]pyridazin-2-yl)acetonitrile (130 mg, 0.267 mmol) in DCM (5 mL) was added BBr₃ (5.35 mL, 1M, 5.35 mmol). The reaction mixture was stirred at room temperature for 12 hours. The reaction mixture was quenched with saturated NaHCO₃ aq. (20 mL), extracted with DCM (30 mL×2), combined the organic layers, washed brine (50 mL×2), dried and concentrated to dryness. The resulting residue was purified by flash column chromatography (DCM:MeOH=20:1) to give the titled compound (80 mg, 63%). MS: M/e 473 (M+1)⁺.

### Step B: 2-(8-((2S,5R)-4-(1-(3-chloroquinoxalin-6-yl)ethyl)-2,5-dimethylpiperazin-1-yl)-5-methyl-6-oxo-5,6-dihydroimidazo[1,2-b]pyridazin-2-yl)acetonitrile

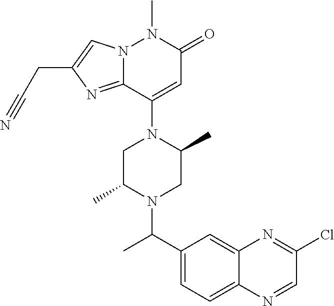 [SMILES: CC(c1ccc2ncc(Cl)nc2c1)N1C[C@H](C)N(c2cc(=O)n(C)n3cc(CC#N)nc23)C[C@H]1C]

A solution of 2-(8-((2S,5R)-4-(1-(3-hydroxyquinoxalin-6-yl)ethyl)-2,5-dimethylpiperazin-1-yl)-5-methyl-6-oxo-5,6-dihydroimidazo[1,2-b]pyridazin-2-yl)acetonitrile (80 mg, 0.169 mmol) in POCl₃ (5 mL) was stirred at 80° C. for 2 hours. The reaction mixture was poured into H₂O (30 mL), extracted with DCM (30 mL×2), combined the organic layers, washed brine (50 mL×2), dried over Na₂SO₄ and concentrated to dryness. The resulting residue was purified by Prep-TLC (DCM:MeOH=15:1) to give the title compound (9 mg, 11%). ¹H NMR (400 MHz, CD₃OD) δ 8.83 (d, J=4.3 Hz, 1H), 8.11 (dd, J=11.6, 8.7 Hz, 1H), 8.05-7.95 (m, 2H), 7.78 (s, 1H), 5.62 (s, 1H), 4.78-4.48 (m, 1H), 4.05-3.94 (m, 0.5H), 3.93 (d, J=2.3 Hz, 2H), 3.87-3.76 (m, 0.5H), 3.74 (s, 3H), 3.71-3.64 (m, 1.5H), 3.49-3.42 (m, 0.5H), 3.13-3.06 (m, 0.5H), 2.95-2.66 (m, 2H), 2.20 (d, J=12.4 Hz, 0.5H), 1.48-1.38 (m, 4H), 1.22 (dd, J=12.2, 6.5 Hz, 4H), 1.06 (d, J=6.5 Hz, 1H) ppm. MS: M/e 491(M+1)⁺.

### Compound A22: 2-(8-((2S,5R)-2,5-dimethyl-4-(1-(3-methylquinoxalin-6-yl)ethyl)piperazin-1-yl)-7-fluoro-5-methyl-6-oxo-5,6-dihydroimidazo[1,2-b]pyridazin-2-yl)acetonitrile

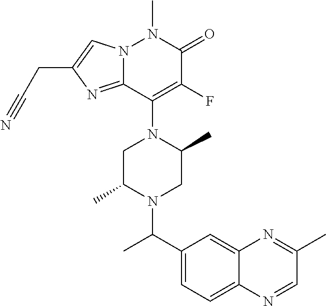 [SMILES: Cc1cnc2ccc(C(C)N3C[C@H](C)N(c4c(F)c(=O)n(C)n5cc(CC#N)nc45)C[C@H]3C)cc2n1]

To a mixture of 2-(8-((2S,5R)-2,5-dimethyl-4-(1-(3-methylquinoxalin-6-yl)ethyl)piperazin-1-yl)-5-methyl-6-oxo-5,6-dihydroimidazo[1,2-b]pyridazin-2-yl)acetonitrile (47 mg, 0.1 mmol) in CH₃CN (2 mL) was added Select F reagent (53 mg, 0.15 mmol). The mixture was stirred at room temperature for 16 hours. The reaction was diluted with water, extracted with DCM (60 mL×2), washed with brine, dried over Na₂SO₄, filtered, and concentrated to dryness. The residue was purified by Prep-TLC (DCM:MeOH=15:1) to give the titled compound (4 mg, 8.3%). ¹H NMR (400 MHz, CD₃OD) δ 8.81-8.76 (m, 1H), 8.07-7.90 (m, 3H), 7.78 (s, 1H), 4.82-4.68 (m, 0.5H), 4.56-4.44 (m, 0.5H), 4.15-3.85 (m, 4.5H), 3.83-3.71 (m, 3.5H), 3.63-3.51 (m, 1H), 3.25-3.16 (m, 0.5H), 2.96-2.79 (m, 1H), 2.76 (s, 3H), 2.24-2.12 (m, 0.5H), 1.49-1.40 (m, 4H), 1.27-1.18 (m, 4H), 1.12-1.04 (m, 1H) ppm. MS: M/e 489 (M+1)⁺.

### Compound A23: 2-(cyanomethyl)-8-((2S,5R)-2,5-dimethyl-4-(1-(3-methylquinoxalin-6-yl)ethyl)piperazin-1-yl)-5-methyl-6-oxo-5,6-dihydroimidazo[1,2-b]pyridazine-7-carbonitrile

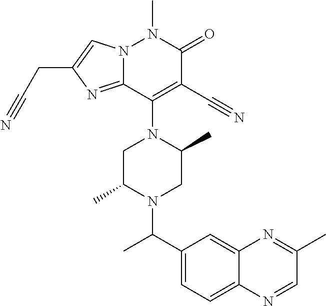 [SMILES: Cc1cnc2ccc(C(C)N3C[C@H](C)N(c4c(C#N)c(=O)n(C)n5cc(CC#N)nc45)C[C@H]3C)cc2n1]

### Step A: 2-(7-bromo-8-((2S,5R)-2,5-dimethyl-4-(1-(3-methylquinoxalin-6-yl)ethyl)piperazin-1-yl)-5-methyl-6-oxo-5,6-dihydroimidazo[1,2-b]pyridazin-2-yl)acetonitrile

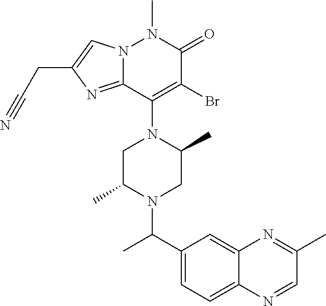 [SMILES: Cc1cnc2ccc(C(C)N3C[C@H](C)N(c4c(Br)c(=O)n(C)n5cc(CC#N)nc45)C[C@H]3C)cc2n1]

To a mixture of 2-(8-((2S,5R)-2,5-dimethyl-4-(1-(3-methylquinoxalin-6-yl)ethyl)piperazin-1-yl)-5-methyl-6-oxo-5,6-dihydroimidazo[1,2-b]pyridazin-2-yl)acetonitrile (200 mg, 0.425 mmol) in CH₃CN (10 mL) was added a solution of NBS (80 mg, 0.43 mmol) in CH₃CN (1 mL). The mixture was stirred at room temperature for 2 hours. The reaction was diluted with water, extracted with DCM (60 mL×2), washed with brine, dried over Na₂SO₄, filtered, and concentrated to dryness. The residue was purified by Prep-TLC (DCM:MeOH=15:1) to give the titled compound (130 mg, 55%). MS: M/e 549 (M+1)⁺.

### Step B: 2-(cyanomethyl)-8-((2S,5R)-2,5-dimethyl-4-(1-(3-methylquinoxalin-6-yl)ethyl)piperazin-1-yl)-5-methyl-6-oxo-5,6-dihydroimidazo[1,2-b]pyridazine-7-carbonitrile

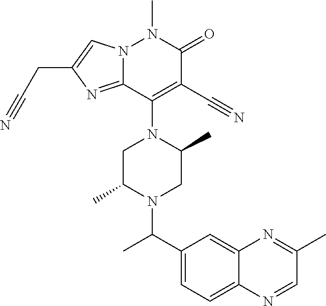 [SMILES: Cc1cnc2ccc(C(C)N3C[C@H](C)N(c4c(C#N)c(=O)n(C)n5cc(CC#N)nc45)C[C@H]3C)cc2n1]

To a mixture of 2-(7-bromo-8-((2S,5R)-2,5-dimethyl-4-(1-(3-methylquinoxalin-6-yl)ethyl)piperazin-1-yl)-5-methyl-6-oxo-5,6-dihydroimidazo[1,2-b]pyridazin-2-yl)acetonitrile (130 mg, 0.236 mmol) in DMF (5 mL) was added Zn(CN)₂ (83 mg, 0.71 mmol) and Pd(PPh₃)₄(82 mg, 0.071 mmol). The mixture was stirred at 100° C. for 16 hours under N₂. The reaction was cooled to room temperature, diluted with water, extracted with DCM (60 mL×2), washed with brine, dried over Na₂SO₄, filtered, and concentrated to dryness. The residue was purified by Prep-TLC (DCM:MeOH=15: 1) to give the titled compound (26 mg, 22%). ¹H NMR (400 MHz, CD₃OD) δ 8.81-8.76 (m, 1H), 8.09-7.90 (m, 3H), 7.86 (s, 1H), 4.22-4.08 (m, 0.5H), 4.01-3.86 (m, 3H), 3.83-3.69 (m, 4H), 3.25-3.16 (m, 1H), 3.06-2.89 (m, 2H), 2.83-2.75 (m, 4H), 2.26-2.19 (m, 0.5H), 1.66 (d, J=6.4 Hz, 1.5H), 1.50-1.38 (m, 4.5H), 1.17 (d, J=6.8 Hz, 1.5H), 1.01 (d, J=6.8 Hz, 1.5H) ppm. MS: M/e 496 (M+1)⁺.

### Compound A24: 2-(8-((2S,5R)-4-(1-(2,3-dihydrobenzo[b][1,4]dioxin-6-yl)ethyl)-2,5-dimethylpiperazin-1-yl)-5-methyl-6-oxo-5,6-dihydroimidazo[1,2-b]pyridazin-2-yl)acetonitrile

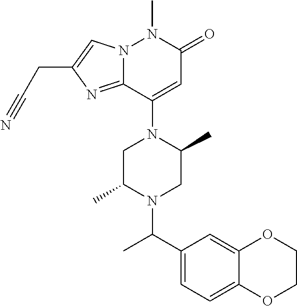 [SMILES: CC(c1ccc2c(c1)OCCO2)N1C[C@H](C)N(c2cc(=O)n(C)n3cc(CC#N)nc23)C[C@H]1C]

A solution of 2-(8-((2S,5R)-2,5-dimethylpiperazin-1-yl)-5-methyl-6-oxo-5,6-dihydroimidazo[1,2-b]pyridazin-2-yl)acetonitrile (300 mg, 1.00 mmol), 1-(2,3-dihydrobenzo[b][1,4]dioxin-6-yl)ethan-1-ol (270 mg, 1.50 mmol), (cyanomethyl)trimethylphosphonium iodide (729 mg, 3.00 mmol) and DIEA (1.29 g, 10.00 mmol) in MeCN (6 ml) was stirred at 100° C. overnight. The reaction was diluted with EA (20 ml) and washed with brine (10 ml). The organic layer was concentrated under reduced pressure. The resulting residue was purified by flash column chromatography with 0-5% MeOH in DCM and then Prep-HPLC to give the titled compound (3.42 mg, FA salt). ¹H NMR (400 MHz, DMSO-d6) δ 8.18 (s, 0.37H), 7.86 (s, 1H), 6.86-6.76 (m, 3H), 5.52 (d, J=3.9 Hz, 1H), 4.22 (d, J=5.8 Hz, 4H), 4.04 (s, 2H), 3.61 (s, 3H), 3.51-3.40 (m, 2H), 3.38-3.35 (m, 1H), 3.23 (d, J=13.0 Hz, 1H), 2.87-2.80 (m, 1H), 2.74 (d, J=11.5 Hz, 0.5H), 2.58 (d, J=8.6 Hz, 0.5H), 2.54-2.52 (m, 0.5H), 2.14 (d, J=11.9 Hz, 0.5H), 1.26 (d, J=6.5 Hz, 1.5H), 1.20 (dd, J=9.3, 6.6 Hz, 3H), 1.11 (d, J=6.3 Hz, 1.5H), 1.03 (d, J=6.2 Hz, 1.5H), 0.88 (d, J=6.5 Hz, 1.5H) ppm. MS: M/e 463 (M+1)⁺.

### Compound A25: 2-(8-((2S,5R)-4-(1-(3,3-dimethyl-2,3-dihydrobenzo[b][1,4]dioxin-6-yl)ethyl)-2,5-dimethylpiperazin-1-yl)-5-methyl-6-oxo-5,6-dihydroimidazo[1,2-b]pyridazin-2-yl)acetonitrile

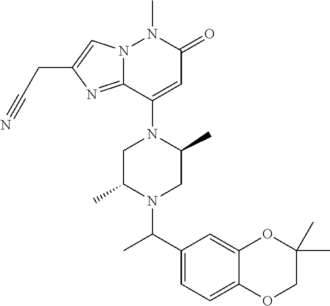 [SMILES: CC(c1ccc2c(c1)OC(C)(C)CO2)N1C[C@H](C)N(c2cc(=O)n(C)n3cc(CC#N)nc23)C[C@H]1C]

A solution of 2-(8-((2S,5R)-2,5-dimethylpiperazin-1-yl)-5-methyl-6-oxo-5,6-dihydroimidazo[1,2-b]pyridazin-2-yl)acetonitrile (150 mg, 0.50 mmol), 1-(3,3-dimethyl-2,3-dihydrobenzo[b][1,4]dioxin-6-yl)ethan-1-ol (125 mg, 0.60 mmol), (cyanomethyl)trimethylphosphonium iodide (365 mg, 1.50 mmol) and DIEA (645 mg, 5 mmol) in MeCN (4 ml) was stirred at 100° C. overnight. The reaction was diluted with EA (20 ml) and washed with brine (10 ml). The organic layer was concentrated under reduced pressure. The resulting residue was purified by flash column chromatography with 0-5% MeOH in DCM to give the titled Compound A25 which was separated into Compound A25a (22 mg) and Compound A25b (23 mg) by Prep-HPLC (Method B) and then chiral prep-HPLC. The chiral separation conditions are shown below.

| | |
|---|---|
| Column | CHIRALPAK IH |
| Column Size | 2 cm × 25 cm, 5 um |
| Mobile Phase A | MtBE |
| Mobile Phase B | EtOH |
| Flow Rate | 20 mL/min |
| Wave Length | UV 220 nm |
| Temperature | 25° C. |
| Prep-HPLC Equipment | Prep-HPLC-Gilson |

Compound A25: ¹H NMR (400 MHz, DMSO-d6) δ 8.18 (s, 0.24H), 7.86 (s, 1H), 6.87-6.76 (m, 3H), 5.52 (d, J=3.0 Hz, 1H), 4.04 (s, 2H), 3.90 (d, J=5.2 Hz, 2H), 3.61 (s, 3H), 3.50-3.37 (m, 4H), 3.23 (d, J=9.7 Hz, 0.5H), 2.87-2.79 (m, 1H), 2.73 (d, J=11.0 Hz, 0.5H), 2.58 (d, J=8.0 Hz, 0.5H), 2.13 (d, J=12.0 Hz, 0.5H), 1.27 (dd, J=7.4, 4.6 Hz, 7.5H), 1.20 (dd, J=10.5, 6.6 Hz, 3H), 1.10 (d, J=6.4 Hz, 1.5H), 1.03 (d, J=6.2 Hz, 1.5H), 0.88 (d, J=6.4 Hz, 1.5H) ppm. MS: M/e 491 (M+1)⁺.

Compound A25a (the earlier peak): ¹H NMR (400 MHz, DMSO-d6) δ 7.86 (s, 1H), 6.86-6.78 (m, 3H), 5.51 (s, 1H), 4.04 (s, 2H), 3.91 (s, 2H), 3.61 (s, 3H), 3.47 (q, J=6.4 Hz, 1H), 3.31 (m, 2H), 3.23 (dd, J=9.4 Hz, 3.6 Hz, 1H), 2.84 (dd, J=7.7 Hz, 3.9 Hz, 2H), 2.73 (d, J=10.5 Hz, 1H), 1.28 (s, 6H), 1.26 (d, J=6.6 Hz, 3H), 1.19 (d, J=6.3 Hz, 3H), 0.89 (d, J=6.4 Hz, 3H) ppm. MS: M/e 491 (M+1)⁺.

Compound A25b (the later peak):¹H NMR (400 MHz, DMSO-d6) δ 7.86 (s, 1H), 6.84-6.76 (m, 3H), 5.52 (s, 1H), 4.04 (s, 2H), 3.89 (s, 2H), 3.61 (s, 3H), 3.50-3.42 (m, 2H), 3.35 (m, 2H), 3.33-3.29 (m, 1H), 2.58 (dd, J=8.9 Hz, 3.8 Hz, 1H), 2.14 (d, J=11.7 Hz, 1H), 1.27 (d, J=3.0 Hz, 6H), 1.22 (d, J=6.3 Hz, 3H), 1.10 (d, J=6.4 Hz, 3H), 1.02 (d, J=6.1 Hz, 3H) ppm. MS: M/e 491 (M+1)⁺.

Compound A26: 2-(8-((2S,5R)-4-(1-(3,3-dimethyl-2,3-dihydrobenzo[b][1,4]dioxin-6-yl)ethyl)-2,5-dimethylpiperazin-1-yl)-7-fluoro-5-methyl-6-oxo-5,6-dihydroimidazo[1,2-b]pyridazin-2-yl)acetonitrile

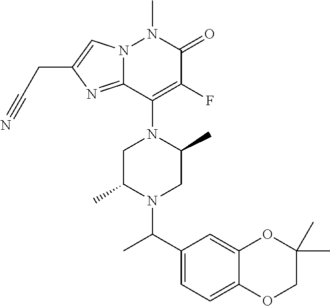 [SMILES: CC(c1ccc2c(c1)OC(C)(C)CO2)N1C[C@H](C)N(c2c(F)c(=O)n(C)n3cc(CC#N)nc23)C[C@H]1C]

### Step A: tert-butyl (2R,5S)-4-(2-(cyanomethyl)-7-fluoro-5-methyl-6-oxo-5,6-dihydroimidazo[1,2-b]pyridazin-8-yl)-2,5-dimethylpiperazine-1-carboxylate

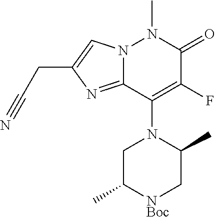 [SMILES: C[C@@H]1CN(c2c(F)c(=O)n(C)n3cc(CC#N)nc23)[C@@H](C)CN1C(=O)OC(C)(C)C]

A solution of tert-butyl (2R,5S)-4-(2-(cyanomethyl)-5-methyl-6-oxo-5,6-dihydroimidazo[1,2-b]pyridazin-8-yl)-2,5-dimethylpiperazine-1-carboxylate (500 mg, 1.25 mmol) and select F (664 mg, 1.88 mmol) in CH₃CN (10 ml) was stirred at rt for 4 h. The solution was diluted with EA (20 ml) and then washed with brine (10 ml). The organic layer was dried over Na₂SO₄ and concentrated under reduced pressure. The resulting residue was purified by flash column chromatography with 60-80% EA in PE to give the titled compound (270 mg, 51%). MS: M/e 419 (M+1)⁺.

### Step B: 2-(8-((2S,5R)-2,5-dimethylpiperazin-1-yl)-7-fluoro-5-methyl-6-oxo-5,6-dihydroimidazo[1,2-b]pyridazin-2-yl)acetonitrile

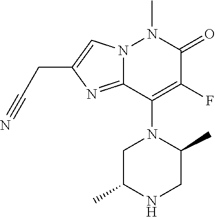 [SMILES: C[C@@H]1CN(c2c(F)c(=O)n(C)n3cc(CC#N)nc23)[C@@H](C)CN1]

A solution of tert-butyl (2R,5S)-4-(2-(cyanomethyl)-7-fluoro-5-methyl-6-oxo-5,6-dihydroimidazo[1,2-b]pyridazin-8-yl)-2,5-dimethylpiperazine-1-carboxylate (270 mg, 0.65 mmol) and TFA (2 ml) in DCM (10 ml) was stirred at rt for 30 min. The solution was diluted with DCM (10 ml) and then washed with aq. NaHCO₃ (10 ml×2). The organic layer was washed with brine (10 ml), dried over Na₂SO₄ and evaporated to dryness to give the titled compound (160 mg, 78%). MS: M/e 319 (M+1)⁺.

### Step C: 2-(8-((2S,5R)-4-(1-(3,3-dimethyl-2,3-dihydrobenzo[b][1,4]dioxin-6-yl)ethyl)-2,5-dimethylpiperazin-1-yl)-7-fluoro-5-methyl-6-oxo-5,6-dihydroimidazo[1,2-b]pyridazin-2-yl)acetonitrile

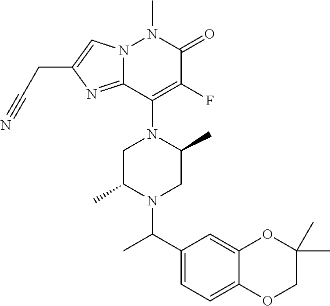 [SMILES: CC(c1ccc2c(c1)OC(C)(C)CO2)N1C[C@H](C)N(c2c(F)c(=O)n(C)n3cc(CC#N)nc23)C[C@H]1C]

A solution of 2-(8-((2S,5R)-2,5-dimethylpiperazin-1-yl)-7-fluoro-5-methyl-6-oxo-5,6-dihydroimidazo[1,2-b]pyridazin-2-yl)acetonitrile (160 mg, 0.50 mmol), 1-(3,3-dimethyl-2,3-dihydrobenzo[b][1,4]dioxin-6-yl)ethan-1-ol (209 mg, 1.00 mmol), (cyanomethyl)trimethylphosphonium iodide (367 mg, 1.50 mmol) and DIEA (649 mg, 5 mmol) in MeCN (4 ml) was stirred at 100° C. overnight. The reaction was diluted with EA (20 ml) and washed with brine (10 ml). The organic layer was concentrated under reduced pressure. The resulting residue was purified by flash column chromatography with 0-5% MeOH in DCM to give the titled compound Compound A26 which was separated into Compound A26a (24 mg) and Compound A26b (24 mg) by chiral prep-HPLC. The chiral separation conditions are shown below.

| | |
|---|---|
| Column | CHIRALPAK IH |
| Column Size | 2 cm × 25 cm, 5 um |
| Mobile Phase A | MtBE |
| Mobile Phase B | MeOH:DCM |
| Flow Rate | 20 mL/min |
| Wave Length | UV 220 nm |
| Temperature | 25° C. |
| Prep-HPLC Equipment | Prep-HPLC-Gilson |

Compound A26: ¹H NMR (400 MHz, DMSO-d6) δ 8.22 (s, 0.17H), 7.87 (s, 1H), 6.86-6.78 (m, 3H), 4.56 (s, 0.5H), 4.39 (s, 0.5H), 4.04 (s, 2H), 3.90 (d, J=5.8 Hz, 2H), 3.78 (d, J=11.8 Hz, 0.5H), 3.70 (s, 0.5H), 3.67 (s, 3H), 3.60 (dd, J=15.3, 9.3 Hz, 1H), 3.48 (q, J=6.5 Hz, 0.5H), 3.38 (s, 0.5H), 2.94 (d, J=8.2 Hz, 0.5H), 2.65 (d, J=12.3 Hz, 1H), 2.54 (s, 1H), 2.10 (d, J=11.1 Hz, 0.5H), 1.29-1.24 (m, 7.5H), 1.22 (t, J=7.2 Hz, 3H), 1.13 (d, J=6.5 Hz, 1.5H), 1.04 (d, J=6.3 Hz, 1.5H), 0.94 (d, J=6.3 Hz, 1.5H) ppm. MS: M/e 509 (M+1)⁺.

Compound A26a (the earlier peak):¹H NMR (400 MHz, DMSO-d6) δ 7.88 (s, 1H), 6.87-6.82 (m, 1H), 6.81-6.77 (m, 2H), 4.57 (s, 1H), 4.05 (s, 2H), 3.91 (s, 2H), 3.67 (s, 3H), 3.64-3.55 (m, 2H), 3.40-3.34 (m, 1H), 3.31 (s, 1H), 2.94 (dd, J=8.3 Hz, 3.6 Hz, 1H), 2.68 (s, 1H), 1.28 (s, 6H), 1.26 (d, J=6.6 Hz, 3H), 1.22 (d, J=6.5 Hz, 3H), 0.94 (d, J=6.4 Hz, 3H) ppm. MS: M/e 509 (M+1)⁺.

Compound A26b (the later peak):¹H NMR (400 MHz, DMSO-d6) δ 7.87 (s, 1H), 6.81 (d, J=4.5 Hz, 3H), 4.40 (s, 1H), 4.04 (s, 2H), 3.89 (s, 2H), 3.77 (d, J=6.7 Hz, 1H), 3.70 (s, 1H), 3.67 (s, 3H), 3.48 (q, J=6.7 Hz, 1H), 3.31 (s, 1H), 2.64 (dd, J=12 Hz, 4 Hz, 1H), 2.09 (dd, J=11.9 Hz, 3.8 Hz, 1H), 1.27 (d, J=3.8 Hz, 6H), 1.20 (d, J=6.5 Hz, 3H), 1.13 (d, J=6.4 Hz, 3H), 1.03 (d, J=6.3 Hz, 3H) ppm. MS: M/e 509 (M+1)⁺.

Compound A27: 2-(cyanomethyl)-8-((2S,5R)-4-(1-(3,3-dimethyl-2,3-dihydrobenzo[b][1,4]dioxin-6-yl)ethyl)-2,5-dimethylpiperazin-1-yl)-5-methyl-6-oxo-5,6-dihydroimidazo[1,2-b]pyridazine-7-carbonitrile

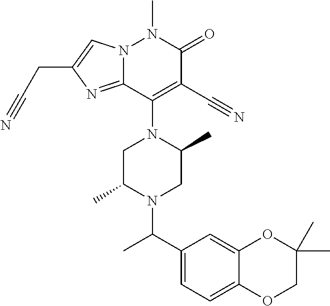 [SMILES: CC(c1ccc2c(c1)OC(C)(C)CO2)N1C[C@H](C)N(c2c(C#N)c(=O)n(C)n3cc(CC#N)nc23)C[C@H]1C]

### Step A: tert-butyl (2R,5S)-4-(7-bromo-2-(cyanomethyl)-5-methyl-6-oxo-5,6-dihydroimidazo[1,2-b]pyridazin-8-yl)-2,5-dimethylpiperazine-1-carboxylate

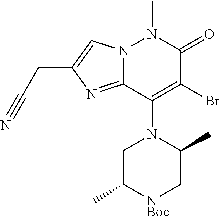 [SMILES: C[C@@H]1CN(c2c(Br)c(=O)n(C)n3cc(CC#N)nc23)[C@@H](C)CN1C(=O)OC(C)(C)C]

A solution of tert-butyl (2R,5S)-4-(2-(cyanomethyl)-5-methyl-6-oxo-5,6-dihydroimidazo[1,2-b]pyridazin-8-yl)-2,5-dimethylpiperazine-1-carboxylate (500 mg, 1.25 mmol) and NBS (245 mg, 1.38 mmol) in CH₃CN (10 ml) was stirred at rt for 0.5 h. The solution was diluted with EA (20 ml) and then washed with brine (10 ml). The organic layer was dried over Na₂SO₄ and concentrated under reduced pressure. The resulting residue was purified by flash column chromatography with 50-80% EA in PE to give the titled compound (598 mg, 100%). MS: M/e 479, 481 (M+1)⁺.

### Step B: tert-butyl (2R,5S)-4-(7-cyano-2-(cyanomethyl)-5-methyl-6-oxo-5,6-dihydroimidazo[1,2-b]pyridazin-8-yl)-2,5-dimethylpiperazine-1-carboxylate

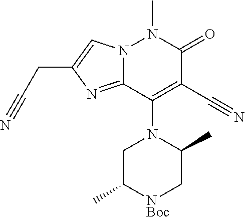 [SMILES: C[C@@H]1CN(c2c(C#N)c(=O)n(C)n3cc(CC#N)nc23)[C@@H](C)CN1C(=O)OC(C)(C)C]

A solution of tert-butyl (2R,5S)-4-(7-bromo-2-(cyanomethyl)-5-methyl-6-oxo-5,6-dihydroimidazo[1,2-b]pyridazin-8-yl)-2,5-dimethylpiperazine-1-carboxylate (598 mg, 1.25 mmol), Zn(CN)₂ (439 mg, 3.75 mmol) and Pd(PPh₃)₄(144 mg, 0.12 mmol) in DMF (10 ml) was stirred at 100° C. for 2 days under N₂. The mixture was poured into water (20 ml) and then extracted with EA (15 ml×2). The organic layer was washed with brine (10 ml), dried over Na₂SO₄ and concentrated under reduced pressure. The resulting residue was purified by flash column chromatography with 60-100% EA in PE to give the titled compound (531 mg, crude). MS: M/e 426 (M+1)⁺.

### Step C: 2-(cyanomethyl)-8-((2S,5R)-2,5-dimethylpiperazin-1-yl)-5-methyl-6-oxo-5,6-dihydroimidazo[1,2-b]pyridazine-7-carbonitrile

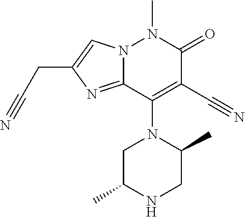 [SMILES: C[C@@H]1CN(c2c(C#N)c(=O)n(C)n3cc(CC#N)nc23)[C@@H](C)CN1]

A solution of tert-butyl (2R,5S)-4-(7-cyano-2-(cyanomethyl)-5-methyl-6-oxo-5,6-dihydroimidazo[1,2-b]pyridazin-8-yl)-2,5-dimethylpiperazine-1-carboxylate (531 mg, crude) and TFA (4 ml) in DCM (16 ml) was stirred at rt for 30 min. The solution was diluted with DCM (15 ml) and then washed with aq. NaHCO₃ (10 ml×2). The organic layer was washed with brine (10 ml), dried over Na₂SO₄ and evaporated to dryness to give the titled compound (406 mg, crude). MS: M/e 326 (M+1)⁺.

### Step D: 2-(cyanomethyl)-8-((2S,5R)-4-(1-(3,3-dimethyl-2,3-dihydrobenzo[b][1,4]dioxin-6-yl)ethyl)-2,5-dimethylpiperazin-1-yl)-5-methyl-6-oxo-5,6-dihydroimidazo[1,2-b]pyridazine-7-carbonitrile

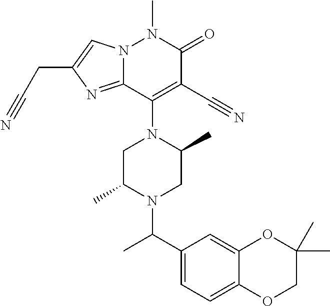 [SMILES: CC(c1ccc2c(c1)OC(C)(C)CO2)N1C[C@H](C)N(c2c(C#N)c(=O)n(C)n3cc(CC#N)nc23)C[C@H]1C]

A solution of 2-(cyanomethyl)-8-((2S,5R)-2,5-dimethylpiperazin-1-yl)-5-methyl-6-oxo-5,6-dihydroimidazo[1,2-b]pyridazine-7-carbonitrile (200 mg, 0.61 mmol), 1-(3,3-dimethyl-2,3-dihydrobenzo[b][1,4]dioxin-6-yl)ethan-1-ol (192 mg, 0.92 mmol), (cyanomethyl)trimethylphosphonium iodide (447 mg, 1.84 mmol) and DIEA (791 mg, 6.13 mmol) in MeCN (6 ml) was stirred at 100° C. overnight. The reaction was diluted with EA (20 ml) and washed with brine (10 ml). The organic layer was concentrated under reduced pressure. The resulting residue was purified by flash column chromatography with 0-5% MeOH in DCM to give the titled compound Compound A27 which was separated into Compound A27a (19 mg) and Compound A27b (24 mg) by chiral prep-HPLC. The chiral separation conditions are shown below.

| | |
|---|---|
| Column | CHIRALPAK IF |
| Column Size | 2 cm × 25 cm, 5 um |
| Mobile Phase A | MtBE |
| Mobile Phase B | MeOH:D CM |
| Flow Rate | 20 mL/min |
| Wave Length | UV 220 nm |
| Temperature | 25° C. |
| Prep-HPLC Equipment | Prep-HPLC-Gilson |

Compound A27:¹H NMR (400 MHz, DMSO-d6) δ 8.00 (s, 1H), 6.86-6.78 (m, 3H), 4.09 (s, 2H), 3.92 (s, 0.5H), 3.90 (d, J=3.2 Hz, 2H), 3.73 (d, J=11.5 Hz, 0.5H), 3.63 (s, 3H), 3.56-3.44 (m, 1H), 3.36 (q, J=6.5 Hz, 0.5H), 2.94 (d, J=9.2 Hz, 0.5H), 2.88-2.79 (m, 1H), 2.73 (d, J=9.8 Hz, 0.5H), 2.54 (s, 2H), 2.17 (d, J=11.8 Hz, 0.5H), 1.47 (d, J=6.6 Hz, 1.5H), 1.33-1.26 (m, 7.5H), 1.21 (t, J=6.2 Hz, 3H), 0.97 (d, J=6.2 Hz, 1.5H), 0.84 (d, J=6.3 Hz, 1.5H) ppm. MS: M/e 516 (M+1)⁺.

Compound A27a (the earlier peak):¹H NMR (400 MHz, DMSO-d6) δ 7.99 (s, 1H), 6.87-6.79 (m, 3H), 4.09 (s, 2H), 3.91 (s, 2H), 3.73 (d, J=11.8 Hz, 1H), 3.63 (s, 3H), 3.49 (q, J=8.1 Hz, 1H), 3.33-3.30 (m, 1H), 2.95 (d, J=8.5 Hz, 1H), 2.89-2.79 (m, 2H), 2.55-2.51 (m, 1H), 1.48 (d, J=6.3 Hz, 3H), 1.28 (d, J=2.4 Hz, 6H), 1.20 (d, J=6.3 Hz, 3H), 0.85 (d, J=6.4 Hz, 3H) ppm. MS: M/e 516 (M+1)⁺.

Compound A27b (the later peak):¹H NMR (400 MHz, DMSO-d6) δ 8.00 (s, 1H), 6.80 (d, J=8.0 Hz, 3H), 4.09 (s, 2H), 3.99-3.91 (m, 1H), 3.90 (s, 2H), 3.63 (s, 3H), 3.55-3.49 (m, 1H), 3.37 (q, J=6.1 Hz, 1H), 3.31 (s, 1H), 2.72 (d, J=8.3 Hz, 1H), 2.53 (m, 1H), 2.18 (d, J=12.5 Hz, 1H), 1.30 (d, J=6.3 Hz, 3H), 1.27 (d, J=3.6 Hz, 6H), 1.22 (d, J=6.4 Hz, 3H), 0.97 (d, J=6.5 Hz, 3H) ppm. MS: M/e 516 (M+1)⁺.

Compound A28:2-(8-((2S,5R)-4-(1-(8-fluoro-3,3-dimethyl-2,3-dihydrobenzo[b][1,4]dioxin-6-yl)ethyl)-2,5-dimethylpiperazin-1-yl)-5-methyl-6-oxo-5,6-dihydroimidazo[1,2-b]pyridazin-2-yl)acetonitrile

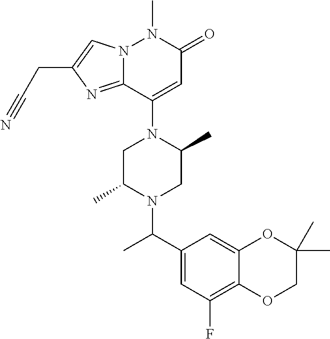 [SMILES: CC(c1cc(F)c2c(c1)OC(C)(C)CO2)N1C[C@H](C)N(c2cc(=O)n(C)n3cc(CC#N)nc23)C[C@H]1C]

### Step A: 8-fluoro-3,3-dimethyl-2,3-dihydrobenzo[b][1,4]dioxine-6-carbonitrile

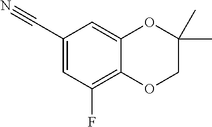

To a solution of 3,4-difluoro-5-hydroxybenzonitrile (1.7 g, 10.9 mmol) and Cs2CO3 (14.3 g, 43.9 mmol) in DMA (50 mL) was added 2,2-dimethyloxirane (6.3 g, 87.2 mol) at room temperature. The reaction mixture was stirred at 100° C. overnight. The reaction mixture was filtered. H₂O (50 mL) was added to the filtrates. The mixture was extracted with PE (100 mL×3). The combined organic layers were concentrated to give the crude product. The crude product was purified by flash column chromatography (PE:EA=5:1) to give the titled compound (1.3 g, 57%). MS: M/e 208 (M+1)⁺.

### Step B: 8-fluoro-3,3-dimethyl-2,3-dihydrobenzo[b][1,4]dioxine-6-carbaldehyde

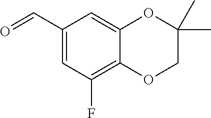

To a solution of 8-fluoro-3,3-dimethyl-2,3-dihydrobenzo[b][1,4]dioxine-6-carbonitrile (1.3 g, 6.3 mmol) in THF (20 mL) was added DIABL-H (9.4 mL, 9.4 mmol, 1M in n-Hexane) slowly at 0° C. The reaction mixture was stirred at 0° C. for 2 hours. The reaction was quenched by 2 N HCl (30 mL). The resulting mixture was stirred at room temperature for 1 hour. The reaction mixture was extracted with EtOAc (50 mL×3). The organic layers were concentrated and purified by flash column chromatography to give the titled compound (0.9 g, 70%). MS: M/e 211 (M+1)⁺.

### Step C: 1-(8-fluoro-3,3-dimethyl-2,3-dihydrobenzo[b][1,4]dioxin-6-yl)ethan-1-ol

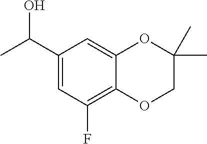 [SMILES: CC(O)c1cc(F)c2c(c1)OC(C)(C)CO2]

To a solution of 8-fluoro-3,3-dimethyl-2,3-dihydrobenzo[b][1,4]dioxine-6-carbaldehyde (0.9 g, 4.3 mmol) in THF (10 mL) was added MeMgBr (2.2 mL, 6.4 mmol, 3M in Et2O) slowly at 0° C. The resulting mixture was stirred at 0° C. for 2 hours. The reaction mixture was quenched with H₂O (10 mL) and extracted with EtOAc (20 mL×3). The combined organic layers were concentrated and purified by flash column chromatography to give the titled compound (0.55 g, 57% yield).

### StepD:2-(8-((2S,5R)-4-(1-(8-fluoro-3,3-dimethyl-2,3-dihydrobenzo[b][1,4]dioxin-6-yl)ethyl)-2,5-dimethylpiperazin-1-yl)-5-methyl-6-oxo-5,6-dihydroimidazo[1,2-b]pyridazin-2-yl)acetonitrile

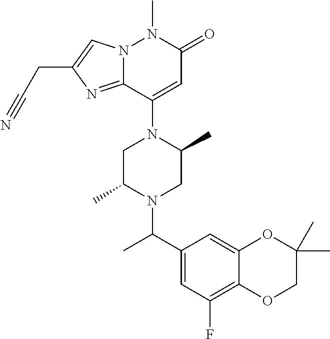 [SMILES: CC(c1cc(F)c2c(c1)OC(C)(C)CO2)N1C[C@H](C)N(c2cc(=O)n(C)n3cc(CC#N)nc23)C[C@H]1C]

A solution of 2-(8-((2S,5R)-2,5-dimethylpiperazin-1-yl)-5-methyl-6-oxo-5,6-dihydroimidazo[1,2-b]pyridazin-2-yl)acetonitrile (60 mg, 0.2 mmol), 1-(8-fluoro-3,3-dimethyl-2,3-dihydrobenzo[b][1,4]dioxin-6-yl)ethan-1-ol (90 mg, 0.4 mmol), (cyanomethyl)trimethylphosphonium iodide (95 mg, 0.4 mmol) and DIPEA (130 mg, 1.0 mmol) in CH₃CN (2 ml) was stirred at 100° C. overnight. After completed, the reaction solution was concentrated under reduced pressure. The resulting residue was purified by Prep-HPLC (Method B) to give the titled compound (27 mg, 26%). ¹H NMR (400 MHz, CD₃OD) δ 7.78 (s, 1H), 6.74-6.66 (m, 2H), 5.62 (s, 1H), 3.97-3.93 (m, 4H), 3.74 (s, 3H), 3.59-3.42 (m, 3H), 2.97-2.80 (m, 2H), 1.39-1.23 (m, 14H), 1.13-1.01 (m, 3H) ppm. MS: M/e 509 (M+1)⁺

### Compound A29: 2-(8-((2S,5R)-4-(1-(benzo[d][1,3]dioxol-5-yl)ethyl)-2,5-dimethylpiperazin-1-yl)-5-methyl-6-oxo-5,6-dihydroimidazo[1,2-b]pyridazin-2-yl)acetonitrile

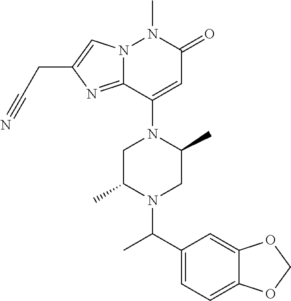 [SMILES: CC(c1ccc2c(c1)OCO2)N1C[C@H](C)N(c2cc(=O)n(C)n3cc(CC#N)nc23)C[C@H]1C]

A solution of 2-(8-((2S,5R)-2,5-dimethylpiperazin-1-yl)-5-methyl-6-oxo-5,6-dihydroimidazo[1,2-b]pyridazin-2-yl)acetonitrile (50 mg, 0.11 mmol), 1-(benzo[d][1,3]dioxol-5-yl)ethan-1-ol (21 mg, 0.13 mmol), (cyanomethyl)trimethylphosphonium iodide (122 mg, 0.5 mmol) and DIPEA (108 mg, 0.83 mmol) in MeCN (10 ml) was stirred at 100° C. overnight. After completed, the solution was concentrated under reduced pressure. The resulting residue was purified by Prep-HPLC (Method A) to give the titled compound (20 mg, 42%). ¹H NMR (400 MHz, CD₃OD) δ 7.78 (s, 1H), 6.93 (s, 1H), 6.82 (t, J=7.0 Hz, 1H), 6.78-6.71 (m, 1H), 5.92 (dd, J=4.4, 3.4 Hz, 2H), 5.61 (d, J=1.4 Hz, 1H), 5.12-4.89 (m, 1H), 4.88-4.38 (m, 2H), 3.94 (s, 2H), 3.73 (s, 3H), 3.60 (d, J=10.1 Hz, 1H), 3.51-3.38 (m, 1H), 2.99 (s, 1H), 2.81 (dd, J=29.9, 12.2 Hz, 1H), 1.40-1.21 (m, 6H), 1.16-0.99 (m, 3H). MS: M/e 449 (M+1)⁺

### Compound A30: 2-(8-((2S,5R)-4-(1-(2,2-dimethylbenzo[d][1,3]dioxol-5-yl)ethyl)-2,5-dimethylpiperazin-1-yl)-5-methyl-6-oxo-5,6-dihydroimidazo[1,2-b]pyridazin-2-yl)acetonitrile

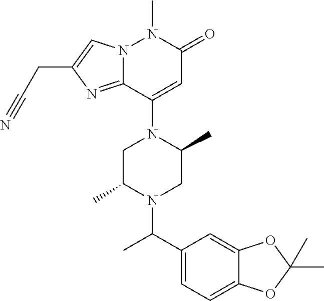 [SMILES: CC(c1ccc2c(c1)OC(C)(C)O2)N1C[C@H](C)N(c2cc(=O)n(C)n3cc(CC#N)nc23)C[C@H]1C]

A solution of 2-(8-((2S,5R)-2,5-dimethylpiperazin-1-yl)-5-methyl-6-oxo-5,6-dihydroimidazo[1,2-b]pyridazin-2-yl)acetonitrile (50 mg, 0.11 mmol), 1-(2,2-dimethylbenzo[d][1,3]dioxol-5-yl)ethan-1-ol (24 mg, 0.13 mmol), (cyanomethyl)trimethylphosphonium iodide (122 mg, 0.5 mmol) and DIPEA (108 mg, 0.83 mmol) in MeCN (10 ml) was stirred at 100° C. overnight. After completed, the solution was concentrated under reduced pressure. The resulting residue was purified by Prep-HPLC (Method A) to give the titled compound (15 mg, 30%).¹H NMR (400 MHz, CD₃OD) δ 7.78 (s, 1H), 6.81 (s, 1H), 6.75 (d, J=7.8 Hz, 1H), 6.68-6.59 (m, 1H), 5.61 (d, J=2.0 Hz, 1H), 4.80-4.48 (m, 1H), 3.94 (s, 2H), 3.73 (s, 3H), 3.64-3.37 (m, 3H), 3.02-2.92 (m, 1H), 2.89-2.64 (m, 2H), 1.66-1.61 (m, 6H), 1.41-1.30 (m, 3H), 1.29-1.21 (m, 3H), 1.14-0.97 (m, 3H). MS: M/e 477 (M+1)⁺

### Compound A31: 2-(8-((2S,5R)-4-(1-(2,2-difluorobenzo[d][1,3]dioxol-5-yl)ethyl)-2,5-dimethylpiperazin-1-yl)-5-methyl-6-oxo-5,6-dihydroimidazo[1,2-b]pyridazin-2-yl)acetonitrile

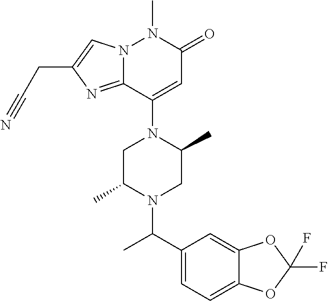 [SMILES: CC(c1ccc2c(c1)OC(F)(F)O2)N1C[C@H](C)N(c2cc(=O)n(C)n3cc(CC#N)nc23)C[C@H]1C]

A solution of 2-(8-((2S,5R)-2,5-dimethylpiperazin-1-yl)-5-methyl-6-oxo-5,6-dihydroimidazo[1,2-b]pyridazin-2-yl)acetonitrile (50 mg, 0.11 mmol), 1-(2,2-difluorobenzo[d][1,3]dioxol-5-yl)ethan-1-ol (25 mg, 0.13 mmol), (cyanomethyl)trimethylphosphonium iodide (122 mg, 0.5 mmol) and DIPEA (108 mg, 0.83 mmol) in MeCN (10 ml) was stirred at 100° C. overnight. After completed, the solution was concentrated under reduced pressure. The resulting residue was purified by Prep-HPLC (Method A) to give the titled compound (22 mg, 43%).¹H NMR (400 MHz, CD₃OD) δ 7.78 (s, 1H), 7.30 (s, 1H), 7.20-7.10 (m, 2H), 5.62 (d, J=1.8 Hz, 1H), 4.88-4.24 (m, 2H), 3.94 (s, 2H), 3.73 (s, 3H), 3.72-3.38 (m, 3H), 3.05-2.79 (m, 2H), 1.41-1.33 (m, 3H), 1.32-1.21 (m, 3H), 1.16-1.00 (m, 3H). MS: M/e 485 (M+1)⁺

### Compound A32: 2-(8-((2S,5R)-2,5-diethyl-4-(1-(4-fluoro-2-methoxyphenyl)ethyl)piperazin-1-yl)-5-methyl-6-oxo-5,6-dihydroimidazo[1,2-b]pyridazin-2-yl)acetonitrile

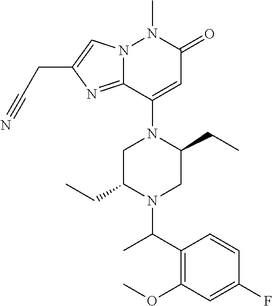 [SMILES: CC[C@H]1CN(C(C)c2ccc(F)cc2OC)[C@H](CC)CN1c1cc(=O)n(C)n2cc(CC#N)nc12]

To a mixture of 2-(8-((2S,5R)-2,5-diethylpiperazin-1-yl)-5-methyl-6-oxo-5,6-dihydroimidazo[1,2-b]pyridazin-2-yl)acetonitrile (65 mg, 0.2 mmol) in acetonitrile (3 mL) was added trimethyl(prop-2-yn-1-yl)phosphonium (97 mg, 0.4 mmol), 1-(4-fluoro-2-methoxyphenyl)ethan-1-ol (68 mg, 0.4 mmol) and DIEA (129 mg, 1 mmol). The reaction mixture was sealed and stirred under nitrogen protection at 105° C. overnight. The mixture was added H₂O and extracted by ethyl acetate. The organic phase was washed with brine, dried over Na₂SO₄, filtered, and concentrated. The crude product was purified by Prep-TLC (DCM:MeOH=13:1) to give the titled compound (10 mg). ¹H NMR (400 MHz, DMSO-d6) δ 7.92 (d, J=3.2 Hz, 1H), 7.65-7.47 (m, 1H), 6.94 (t, J=10.7 Hz, 1H), 6.84 (q, J=8.2 Hz, 1H), 5.57 (s, 1H), 4.23 (d, J=6.6 Hz, 0.5H), 4.09 (s, 2H), 4.05 (d, J=6.3 Hz, 0.5H), 3.86 (s, 3H), 3.67 (s, 3H), 3.20-3.05 (m, 1H), 2.88-2.78 (m, 1H), 2.40-2.27 (m, 1H), 2.05-1.70 (m, 2H), 1.68-1.35 (m, 3H), 1.30 (s, 1H), 1.24 (dd, J=21.1, 6.5 Hz, 3H), 1.2-0.81 (m, 4H), 0.69 (d, J=19.6 Hz, 3H) ppm. MS: M/e 481 (M+1)⁺.

### Compound A33: 2-(8-((2S,5R)-4-(1-(2-(difluoromethoxy)-4-fluorophenyl)ethyl)-2,5-diethylpiperazin-1-yl)-5-methyl-6-oxo-5,6-dihydroimidazo[1,2-b]pyridazin-2-yl)acetonitrile

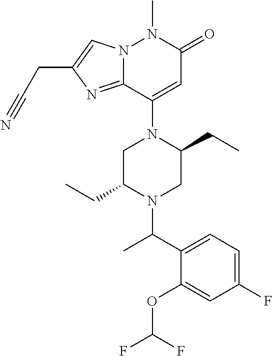 [SMILES: CC[C@H]1CN(C(C)c2ccc(F)cc2OC(F)F)[C@H](CC)CN1c1cc(=O)n(C)n2cc(CC#N)nc12]

### Step A: 1-(2-(difluoromethoxy)-4-fluorophenyl)ethan-1-one

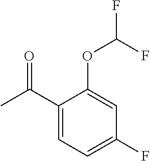

A mixture of 1-bromo-2-(difluoromethoxy)-4-fluorobenzene (2.4 g, 10 mmol), tributyl(1-ethoxyvinyl)stannane (4.3 g, 12 mmol) and Pd(PPh₃)₂Cl₂ (701 mg, 1 mmol) in toluene (20 mL) was stirred at 100° C. under N₂ overnight. To the resulting solution was added HCl (1N, 50 ml) in drops and the mixture was stirred at room temperature for 1 hour. The mixture was diluted with EtOAc, treated with saturated NaHCO₃ aq. to pH ˜8, washed with brine, dried and concentrated to dryness. The resulting residue was purified by flash column chromatography to give the titled compound (1.6 g, 78%). MS: M/e 205 (M+1)⁺.

### Step B: 1-(2-(difluoromethoxy)-4-fluorophenyl)ethan-1-ol

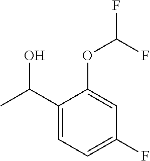

To a solution of 1-(2-(difluoromethoxy)-4-fluorophenyl)ethan-1-one (1.6 g, 7.8 mmol) in MeOH (10 mL) was added NaBH₄ (300 mg, 7.8 mmol) at room temperature and the mixture was stirred at room temperature for 30 minutes. The mixture was quenched with water and extracted with DCM. The combined organic layers were washed with brine, dried over Na₂SO₄ and concentrated to dryness. The resulting residue was purified by flash column chromatography to give the titled compound (1.2 g, 75%). MS: M/e 207 (M+1)⁺.

### Step C: 2-(8-((2S,5R)-4-(1-(2-(difluoromethoxy)-4-fluorophenyl)ethyl)-2,5-diethylpiperazin-1-yl)-5-methyl-6-oxo-5,6-dihydroimidazo[1,2-b]pyridazin-2-yl)acetonitrile

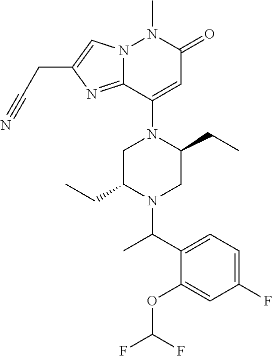 [SMILES: CC[C@H]1CN(C(C)c2ccc(F)cc2OC(F)F)[C@H](CC)CN1c1cc(=O)n(C)n2cc(CC#N)nc12]

To a solution of 1-(2-(difluoromethoxy)-4-fluorophenyl)ethan-1-ol (82 mg, 0.4 mmol), 2-(8-((2S,5R)-2,5-diethylpiperazin-1-yl)-5-methyl-6-oxo-5,6-dihydroimidazo[1,2-b]pyridazin-2-yl)acetonitrile (65 mg, 0.2 mmol) and (cyanomethyl)trimethylphosphonium iodide (97 mg, 0.4 mmol) in CH₃CN (3 mL) was added DIPEA (129 mg, 1 mmol). The mixture was sealed in a bottle and heated at 100° C. overnight. Then the mixture was cooled to room temperature, diluted with water, extracted with EtOAc, washed with brine, dried over Na₂SO₄, filtered, and concentrated to dryness. The resulting residue was purified by flash column chromatography (DCM:MeOH=13:1) to give the titled compound (21 mg, 20%). ¹HNMR (400 MHz, DMSO-d6) δ 7.86 (d, J=2.5 Hz, 1H), 7.71-7.57 (m, 1H), 7.52-7.16 (m, 1H), 7.17-7.06 (m, 2H), 5.52 (s, 1H), 4.16-3.93 (m, 3H), 3.60 (s, 3H), 3.33 (s, 3H), 3.16-3.00 (m, 1H), 2.90-2.75 (m, 1H), 2.29-2.14 (m, 1H), 2.02-1.80 (m, 1H), 1.71-1.37 (m, 3H), 1.22 (dd, J=18.6, 6.4 Hz, 3H), 0.99-0.82 (m, 3H), 0.70-0.52 (m, 3H) ppm. MS: M/e 517 (M+1)⁺.

### Compound A34: 2-(8-((2S,5R)-4-(1-(2-(difluoromethyl)-4-fluorophenyl)ethyl)-2,5-diethylpiperazin-1-yl)-5-methyl-6-oxo-5,6-dihydroimidazo[1,2-b]pyridazin-2-yl)acetonitrile

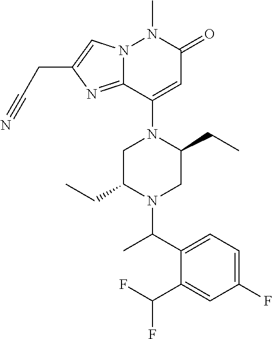 [SMILES: CC[C@H]1CN(C(C)c2ccc(F)cc2C(F)F)[C@H](CC)CN1c1cc(=O)n(C)n2cc(CC#N)nc12]

### Step A: 1-(2-(difluoromethyl)-4-fluorophenyl)ethan-1-one

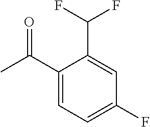

A mixture of 1-bromo-2-(difluoromethyl)-4-fluorobenzene (1.12 g, 5 mmol), tributyl(1-ethoxyvinyl)stannane (2.17 g, 6 mmol) and Pd(PPh₃)₂Cl₂ (350 mg, 0.5 mmol) in toluene (10 mL) was stirred at 100° C. under N2 overnight. To the resulting solution was added HCl (1N, 30 ml) in dropwise and the mixture was stirred at room temperature for 1 hour. The mixture was diluted with EtOAc, treated with saturated NaHCO₃ aq. to pH ˜ 8, washed with brine, dried and concentrated to dryness. The resulting residue was purified by flash column chromatography to give the titled compound (800 mg, 85%). ¹H NMR (400 MHz, CD₃OD) δ 8.10 (dd, J=8.2, 5.9 Hz, 1H), 7.52 (d, J=9.5 Hz, 1H), 7.49-7.19 (m, 2H), 2.66-2.53 (m, 3H). MS: M/e 189 (M+1)⁺.

### Step B: 1-(2-(difluoromethyl)-4-fluorophenyl)ethan-1-ol

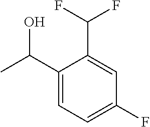

To a solution of 1-(2-(difluoromethyl)-4-fluorophenyl)ethan-1-ol (400 mg, 2.13 mmol) in MeOH (5 mL) was added NaBH₄ (65 mg, 1.7 mmol) at room temperature and the mixture was stirred at room temperature for 30 minutes. The mixture was quenched with water and extracted with DCM. The combined organic layers were washed with brine, dried over Na₂SO₄ and concentrated to dryness. The resulting residue was purified by flash column chromatography to give the titled compound (190 mg, 48%). MS: M/e 191 (M+1)⁺.

### Step C: 2-(8-((2S,5R)-4-(1-(2-(difluoromethyl)-4-fluorophenyl)ethyl)-2,5-diethylpiperazin-1-yl)-5-methyl-6-oxo-5,6-dihydroimidazo[1,2-b]pyridazin-2-yl)acetonitrile

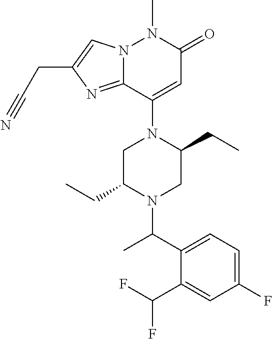 [SMILES: CC[C@H]1CN(C(C)c2ccc(F)cc2C(F)F)[C@H](CC)CN1c1cc(=O)n(C)n2cc(CC#N)nc12]

To a solution of 1-(2-(difluoromethyl)-4-fluorophenyl)ethan-1-ol (57 mg, 0.3 mmol), 2-(8-((2S,5R)-2,5-diethylpiperazin-1-yl)-5-methyl-6-oxo-5,6-dihydroimidazo[1,2-b]pyridazin-2-yl)acetonitrile (65 mg, 0.2 mmol) and (cyanomethyl)trimethylphosphonium iodide (97 mg, 0.4 mmol) in CH₃CN (2 mL) was added DIPEA (103 mg, 0.8 mmol). The mixture was sealed in a bottle and heated at 100° C. overnight. Then the mixture was cooled to room temperature, diluted with water, extracted with EtOAc, washed with brine, dried over Na₂SO₄, filtered, and concentrated to dryness. The resulting residue was purified by flash column chromatography (DCM:MeOH=13:1) to give the titled compound (17 mg, 17%). ¹H NMR (400 MHz, CD₃OD) δ 7.86-7.62 (m, 2H), 7.58-7.17 (m, 3H), 5.61 (d, J=9.5 Hz, 1H), 4.14-3.90 (m, 3H), 3.73 (s, 3H), 3.48 (d, J=13.5 Hz, 1H), 3.27 (s, 2H), 3.21-3.04 (m, 1H), 2.90-2.66 (m, 1H), 2.40-2.21 (m, 1H), 2.09 (s, 1H), 1.87-1.47 (m, 3H), 1.33 (dd, J=14.1, 6.5 Hz, 3H), 1.09-0.93 (m, 3H), 0.73-0.55 (m, 3H) ppm. MS: M/e 501 (M+1)⁺.

### Compound A35: 2-(8-((2S,5R)-2,5-diethyl-4-(1-(pyridin-4-yl)ethyl)piperazin-1-yl)-5-methyl-6-oxo-5,6-dihydroimidazo[1,2-b]pyridazin-2-yl)acetonitrile

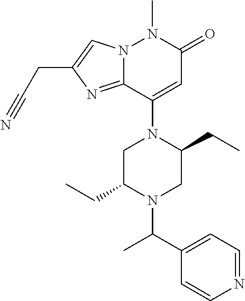 [SMILES: CC[C@H]1CN(C(C)c2ccncc2)[C@H](CC)CN1c1cc(=O)n(C)n2cc(CC#N)nc12]

### Step A: 1-(pyridin-4-yl)ethan-1-ol

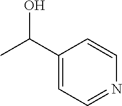

To a solution of 1-(pyridin-4-yl)ethan-1-one (1.2 g, 10 mmol) in EtOH (10 mL) was added NaBH₄ (190 mg, 5 mmol) at 0° C. and the mixture was stirred at room temperature for 30 minutes. The mixture was quenched with water and extracted with DCM. The combined organic layers were washed with brine, dried over Na₂SO₄ and concentrated to dryness. The resulting residue was purified by flash column chromatography to give the titled compound (900 mg, 75%). MS: M/e 124 (M+1)⁺.

### Step B: 2-(8-((2S,5R)-2,5-diethyl-4-(1-(pyridin-4-yl)ethyl)piperazin-1-yl)-5-methyl-6-oxo-5,6-dihydroimidazo[1,2-b]pyridazin-2-yl)acetonitrile

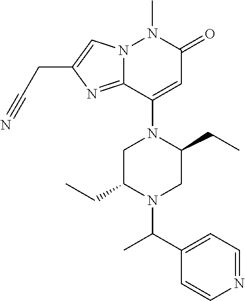 [SMILES: CC[C@H]1CN(C(C)c2ccncc2)[C@H](CC)CN1c1cc(=O)n(C)n2cc(CC#N)nc12]

To a solution of 1-(pyridin-4-yl)ethan-1-ol (37 mg, 0.3 mmol), 2-(8-((2S,5R)-2,5-diethylpiperazin-1-yl)-5-methyl-6-oxo-5,6-dihydroimidazo[1,2-b]pyridazin-2-yl)acetonitrile (65 mg, 0.2 mmol) and (cyanomethyl)trimethylphosphonium iodide (97 mg, 0.4 mmol) in CH₃CN (3 mL) was added DIPEA (129 mg, 1 mmol). The mixture was sealed in a bottle and heated at 100° C. overnight. Then the mixture was cooled to room temperature, diluted with water, extracted with EtOAc, washed with brine, dried over Na₂SO₄, filtered, and concentrated to dryness. The resulting residue was purified by flash column chromatography (DCM:MeOH=13:1) to give the titled compound (5 mg, 6%). ¹HNMR (400 MHz, CDCl₃) δ 8.58 (s, 2H), 7.35 (s, 3H), 5.60 (s, 1H), 3.78 (d, J=2.5 Hz, 2H), 3.75-3.55 (m, 4H), 3.46-3.20 (m, 1H), 3.10-2.90 (m, 1H), 2.88-2.61 (m, 1H), 2.40-2.22 (m, 1H), 2.16-1.83 (m, 2H), 1.82-1.53 (m, 3H), 1.46 (s, 1H), 1.35-1.27 (m, 3H), 0.94 (dd, J=20.8, 13.6 Hz, 3H), 0.67 (dt, J=34.0, 7.3 Hz, 3H) ppm. MS: M/e 434 (M+1)⁺.

### Compound A36: 2-(8-((2S,5R)-2,5-diethyl-4-(4-(trifluoromethoxy)benzyl)piperazin-1-yl)-5-methyl-6-oxo-5,6-dihydroimidazo[1,2-b]pyridazin-2-yl)acetonitrile

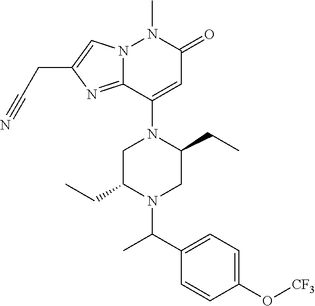 [SMILES: CC[C@H]1CN(C(C)c2ccc(OC(F)(F)F)cc2)[C@H](CC)CN1c1cc(=O)n(C)n2cc(CC#N)nc12]

To a solution of 2-(8-((2S,5R)-2,5-diethylpiperazin-1-yl)-5-methyl-6-oxo-5,6-dihydroimidazo[1,2-b]pyridazin-2-yl)acetonitrile (65 mg, 0.2 mmol) and 1-(bromomethyl)-4-(trifluoromethoxy)benzene (76 mg, 0.3 mmol) in CH₃CN (2 mL) was added DIPEA (77 mg, 0.6 mmol). The reaction mixture was sealed in a bottle and heated at 90° C. for 2 hours, and then cooled to room temperature, diluted with water, extracted with EtOAc. The combined organic layers were washed with brine, dried over Na₂SO₄, filtered, and concentrated to dryness. The resulting residue was purified by Prep-TLC (DCM:MeOH=15: 1) to give the titled compound (29 mg, 29%).¹H (400 MHz, DMSO-d6) δ 7.93 (s, 1H), 7.55 (d, J=8.5 Hz, 2H), 7.39 (d, J=8.0 Hz, 2H), 5.60 (s, 1H), 4.10 (s, 2H), 3.78 (d, J=14.0 Hz, 1H), 3.66 (d, J=11.3 Hz, 4H), 3.41 (d, J=10.7 Hz, 3H), 2.86-2.67 (m, 2H), 2.46 (d, J=11.3 Hz, 1H), 1.94 (dt, J=15.4, 7.6 Hz, 1H), 1.70 (dd, J=13.3, 6.8 Hz, 1H), 1.59 (d, J=7.4 Hz, 2H), 0.93 (t, J=7.2 Hz, 3H), 0.74 (t, J=7.3 Hz, 3H) ppm. MS: M/e 503 (M+1)⁺.

### Compound A37: 2-(8-((2S,5R)-4-(1-(6-cyclopropylpyridin-3-yl)ethyl)-2,5-diethylpiperazin-1-yl)-5-methyl-6-oxo-5,6-dihydroimidazo[1,2-b]pyridazin-2-yl)acetonitrile

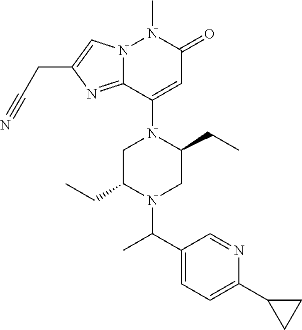 [SMILES: CC[C@H]1CN(C(C)c2ccc(C3CC3)nc2)[C@H](CC)CN1c1cc(=O)n(C)n2cc(CC#N)nc12]

### Step A: 1-(6-cyclopropylpyridin-3-yl)ethan-1-one

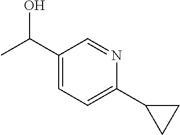

A mixture of 1-(6-bromopyridin-3-yl)ethan-1-one (1 g, 5 mmol), cyclopropylboronic acid (473 mg, 5.5 mmol), tricyclohexylphosphane (140 mg, 0.5 mmol), potassium phosphate (1.6 g, 7.5 mmol) and Pd(OAc)₂ (112 mg, 0.5 mmol) in toluene (15 mL) and water (1.5 ml) was stirred at 100° C. under N₂ overnight. The mixture was treated with water (50 ml), extracted with EtOAc (20 ml×3), washed with brine (50 mL), dried and concentrated to dryness. The resulting residue was purified by flash column chromatography (0-50% EtOAc in PE in 25 minutes) to give the titled compound (620 mg, 77%). MS: M/e 162 (M+1)⁺.

### Step B: 1-(6-cyclopropylpyridin-3-yl)ethan-1-ol

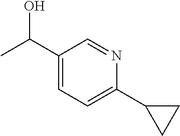

To a solution of 1-(6-cyclopropylpyridin-3-yl)ethan-1-one (620 mg, 3.8 mmol) in MeOH (10 mL) was added NaBH₄ (117 mg, 3.1 mmol) at 0° C. and the mixture was stirred at 0° C. for 30 minutes. The resulting mixture was treated with water (100 ml), extracted with DCM (20 mL×2). The combined organic layers were dried over Na₂SO₄ and concentrated to dryness. The resulting residue was purified by flash column chromatography (0-100% EtOAc in PE in 25 minutes) to give the titled compound (420 mg, 68%). MS: M/e 164 (M+1)⁺.

### Step C: 2-(8-((2S,5R)-4-(1-(6-cyclopropylpyridin-3-yl)ethyl)-2,5-diethylpiperazin-1-yl)-5-methyl-6-oxo-5,6-dihydroimidazo[1,2-b]pyridazin-2-yl)acetonitrile

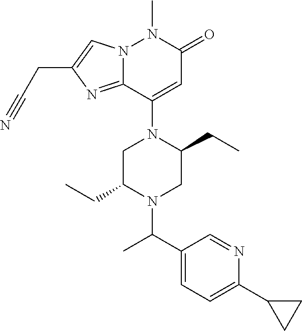 [SMILES: CC[C@H]1CN(C(C)c2ccc(C3CC3)nc2)[C@H](CC)CN1c1cc(=O)n(C)n2cc(CC#N)nc12]

A mixture of 2-(8-((2S,5R)-2,5-diethylpiperazin-1-yl)-5-methyl-6-oxo-5,6-dihydroimidazo[1,2-b]pyridazin-2-yl)acetonitrile (32.8 mg, 0.1 mmol), 1-(6-cyclopropylpyridin-3-yl)ethan-1-ol (32.6 mg, 0.2 mmol), (cyanomethyl)trimethylphosphonium iodide (72.9 mg, 0.3 mmol) and DIPEA (129 mg, 1 mmol) in CH₃CN (4 mL) was stirred overnight at 100° C. in a sealed tube. The reaction mixture was diluted with EtOAc (15 mL), washed with H₂O, brine, dried over Na₂SO₄, concentrated to dryness. The resulting residue was purified by Pre-TLC (CH₂Cl₂/MeOH=10/1) to give the titled Compound A37, which was further separated into Compound A37a (5 mg) and Compound A37b (5 mg) by Prep-HPLC (Method A).

Compound A37a (the earlier peak): ¹H NMR (500 MHz, CD₃OD) δ 8.30 (d, J=2.0 Hz, 1H), 7.78 (s, 1H), 7.70 (dd, J=8.0, 2.0 Hz, 1H), 7.21 (d, J=8.0 Hz, 1H), 5.60 (s, 1H), 3.92 (s, 2H), 3.82-3.76 (m, 1H), 3.73 (s, 3H), 3.30-3.25 (m, 3H), 3.00-2.86 (m, 2H), 2.41-2.34 (m, 1H), 2.13-2.02 (m, 2H), 1.86-1.78 (m, 1H), 1.57-1.48 (m, 2H), 1.35 (d, J=6.4 Hz, 3H), 1.05-0.92 (m, 7H), 0.72 (t, J=7.2 Hz, 3H) ppm. MS: M/e 474(M+1)⁺.

Compound A37b (the later peak): ¹H NMR (500 MHz, CD₃OD) δ 8.33 (d, J=2.0 Hz, 1H), 7.78 (s, 1H), 7.72 (dd, J=8.0, 2.0 Hz, 1H), 7.18 (d, J=8.0 Hz, 1H), 5.59 (s, 1H), 3.93 (s, 2H), 3.73 (s, 3H), 3.67-3.60 (m, 1H), 3.52-3.46 (m, 1H), 3.35-3.30 (m, 2H), 3.17-3.11 (m, 1H), 2.71-2.65 (m, 1H), 2.36-2.29 (m, 1H), 2.11-2.05 (m, 1H), 1.95-1.86 (m, 1H), 1.72-1.50 (m, 3H), 1.32 (d, J=6.4 Hz, 3H), 1.07-0.89 (m, 7H), 0.63 (t, J=7.2 Hz, 3H) ppm. MS: M/e 474(M+1)⁺.

Compound A38: 2-(8-((2S,5R)-2,5-diethyl-4-(1-(6-isopropylpyridin-3-yl)ethyl)piperazin-1-yl)-5-methyl-6-oxo-5,6-dihydroimidazo[1,2-b]pyridazin-2-yl)acetonitrile

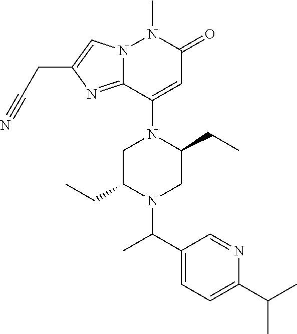 [SMILES: CC[C@H]1CN(C(C)c2ccc(C(C)C)nc2)[C@H](CC)CN1c1cc(=O)n(C)n2cc(CC#N)nc12]

### Step A: 1-(6-isopropylpyridin-3 yl)ethan-1-one

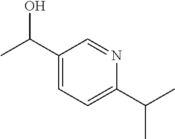

To a stirred solution of 1-(6-bromopyridin-3-yl)ethan-1-one (2 g, 10 mmol) in dry THF (30 mL) was added XantphosPdG3 (190 mg, 0.2 mmol), followed by isopropylzinc(II) bromide (0.5 M, 30 mL, 15 mmol) under N₂. After the addition, the reaction mixture was stirred at 30° C. overnight in a sealed tube. The reaction mixture was added dropwise to MeOH to quench the reaction. Most solvent was removed to give the residue, then treated with EtOAc (200 mL), and washed with brine, dried over Na₂SO₄, concentrated and purified by flash column chromatography to give the titled compound (1.17 g, 71.8%). MS: M/e 164 (M+1)⁺.

### Step B: 1-(6-isopropylpyridin-3-yl)ethan-1-ol

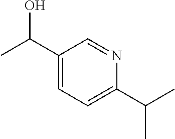

To a stirred solution of 1-(6-isopropylpyridin-3-yl)ethan-1-one (660 mg, 4 mmol) in MeOH (25 mL) was added NaBH₄ (153 mg, 4 mmol). After then, the reaction mixture was stirred for 30 min. The reaction mixture was quenched with H₂O, then most MeOH was removed to give the aqueous layer, then extracted with EtOAc (100 mL×3). The combined organic layers were washed with brine, dried over Na₂SO₄, concentrated to give the titled compound (580 mg, 87.8%). MS: M/e 166 (M+1)⁺.

### Step C: 2-(8-((2S,5R)-2,5-diethyl-4-(1-(6-isopropylpyridin-3-yl)ethyl)piperazin-1-yl)-5-methyl-6-oxo-5,6-dihydroimidazo[1,2-b]pyridazin-2-y)acetonitrile

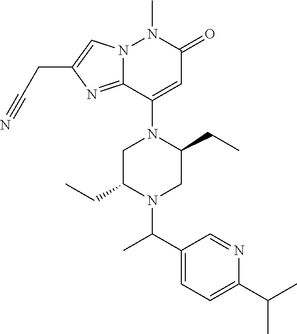 [SMILES: CC[C@H]1CN(C(C)c2ccc(C(C)C)nc2)[C@H](CC)CN1c1cc(=O)n(C)n2cc(CC#N)nc12]

A mixture of 1-(6-isopropylpyridin-3-yl)ethan-1-ol (201 mg, 1.22 mmol), 2-(8-((2S,5R)-2,5-diethylpiperazin-1-yl)-5-methyl-6-oxo-5,6-dihydroimidazo[1,2-b]pyridazin-2-yl) acetonitrile (200 mg, 0.61 mmol), (cyanomethyl)trimethylphosphonium iodide (590 mg, 2.44 mmol) and DIPEA (314 mg, 2.44 mmol) in MeCN (5 mL) was stirred at 100° C. overnight. The reaction mixture was diluted with water (50 mL), extracted with water (10 mL×2), dried over Na₂SO₄ and concentrated to dryness. The resulting residue was purified by flash column chromatography (0-10% MeOH in DCM in 30 minutes) to give the titled Compound A38 (200 mg), which was further separated into Compound A38a (46 mg) and Compound A38b (68 mg) by Prep-HPLC (Method A).

Compound A38a (the earlier peak): ¹H NMR (400 MHz, DMSO-d₆) δ 8.41 (s, 1H), 7.83 (s, 1H), 7.65 (d, J=8.1 Hz, 1H), 7.23 (d, J=8.0 Hz, 1H), 5.49 (s, 1H), 4.00 (s, 2H), 3.72 (q, J=6.1 Hz, 1H), 3.58 (s, 3H), 3.12 (d, J=12.1 Hz, 1H), 2.98 (dt, J=13.7, 6.8 Hz, 1H), 2.86 (d, J=11.9 Hz, 1H), 2.75 (d, J=8.7 Hz, 1H), 2.52 (s, 2H), 2.25 (s, 1H), 2.03-1.88 (m, 1H), 1.70-1.56 (m, 1H), 1.50-1.35 (m, 2H), 1.27 (d, J=6.3 Hz, 3H), 1.21 (d, J=6.9 Hz, 6H), 0.83 (t, J=7.1 Hz, 3H), 0.61 (t, J=7.0 Hz, 3H) ppm. MS: M/e 476 (M+1)⁺.

Compound A38b (the later peak): ¹H NMR (400 MHz, DMSO-d₆) δ 8.42 (s, 1H), 7.84 (s, 1H), 7.71-7.61 (m, 1H), 7.21 (d, J=8.0 Hz, 1H), 5.48 (s, 1H), 4.01 (s, 2H), 3.58 (s, 4H), 3.33 (d, J=11.5 Hz, 2H), 2.99 (dt, J=13.7, 8.2 Hz, 2H), 2.52 (s, 2H), 2.16 (d, J=11.9 Hz, 1H), 1.78 (d, J=6.9 Hz, 1H), 1.47 (dd, J=20.0, 7.8 Hz, 3H), 1.22 (dd, J=16.8, 6.3 Hz, 9H), 0.94 (t, J=7.0 Hz, 3H), 0.49 (s, 3H) ppm. MS: M/e 476 (M+1)⁺.

Compound A39: 5-(1-((2R,5S)-4-(2-(cyanomethyl)-5-methyl-6-oxo-5,6-dihydroimidazo[1,2-b]pyridazin-8-yl)-2,5-diethylpiperazin-1-yl)ethyl)picolinonitrile

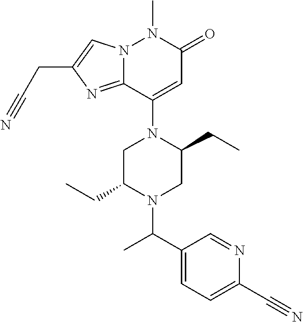 [SMILES: CC[C@H]1CN(C(C)c2ccc(C#N)nc2)[C@H](CC)CN1c1cc(=O)n(C)n2cc(CC#N)nc12]

### Step A: 5-acetylpicolinonitrile

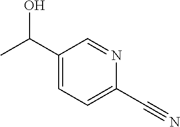

A mixture of 1-(6-bromopyridin-3-yl)ethan-1-one (2.0 g, 10 mmol) and CuCN (1.79 g, 20 mmol) in DMF (20 mL) was stirred at 110° C. for 16 hours. The mixture was diluted with EtOAc (50 mL), filtered through a celite pad and the filtrate was washed with brine (20 mL×3), dried over Na₂SO₄, filtered and concentrated to dryness. The resulted residue was purified by column chromatography to give the titled compound (700 mg, 48%). ¹H NMR (400 MHz, CDCl₃) δ 9.22 (s, 1H), 8.36 (d, J=8.0 Hz, 1H), 7.83 (d, J=8.0 Hz, 1H), 2.69 (s, 3H) ppm.

### Step B: 5-(1-hydroxyethyl)picolinonitrile

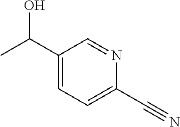

To a 0° C. solution of 5-acetylpicolinonitrile (350 mg, 2.4 mmol) in MeOH (5 mL) was added NaBH₄ (91 mg, 2.4 mmol) in portions and the mixture was stirred at RT for 1 hour. The mixture was treated with NaHCO₃ (20 mL), extracted with EtOAc (15 mL×3). The combined extracts was washed with brine (15 mL×2), dried over Na₂SO₄, filtered and concentrated to dryness to give the titled compound (340 mg, 95%). ¹H NMR (400 MHz, DMSO-d₆) δ 8.71 (s, 1H), 8.09-7.89 (m, 2H), 5.56 (d, J=4.4 Hz, 1H), 4.95-4.79 (m, 1H), 1.35 (d, J=6.4 Hz, 3H) ppm.

### Step C: 5-(1-((2R,5S)-4-(2-(cyanomethyl)-5-methyl-6-oxo-5,6-dihydroimidazo[1,2-b]pyridazin-8-yl)-2,5-diethylpiperazin-1-yl)ethyl)picolinonitrile

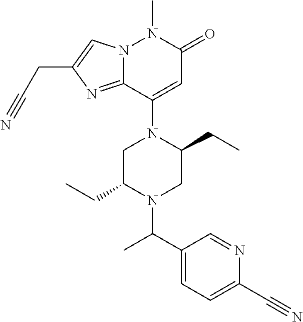 [SMILES: CC[C@H]1CN(C(C)c2ccc(C#N)nc2)[C@H](CC)CN1c1cc(=O)n(C)n2cc(CC#N)nc12]

To a solution of 2-(8-((2S,5R)-2,5-diethylpiperazin-1-yl)-5-methyl-6-oxo-5,6-dihydroimidazo[1,2-b]pyridazin-2-yl)acetonitrile (33 mg, 0.1 mmol), 5-(1-hydroxyethyl)picolinonitrile (30 mg, 0.2 mmol) and (cyanomethyl)trimethylphosphonium iodide (73 mg, 0.30 mmol) in CH₃CN (1 mL) was added DIPEA (77 mg, 0.60 mmol). The mixture was stirred at 105° C. in a sealed tube for 16 hours. Another (cyanomethyl)trimethylphosphonium iodide (73 mg, 0.30 mmol) and DIPEA (77 mg, 0.60 mmol) were added. And the resulted mixture was continue stirred at 105° C. for 20 hours. The mixture was cooled and diluted with EtOAc (10 mL), washed with brine (5 mL×3), dried over Na₂SO₄, filtered and concentrated under reduced pressure. The resulting residue was purified by column chromatography to give the titled compound (16 mg, 35%). ¹H NMR (400 MHz, CD₃OD) δ 8.83-8.66 (m, 1H), 8.14-7.98 (m, 1H), 7.90-7.82 (m, 1H), 7.79 (s, 1H), 5.70-5.53 (m, 1H), 4.03-3.77 (m, 3H), 3.73 (s, 3H), 3.60-3.36 (m, 1H), 3.25-3.08 (m, 1H), 3.06-2.86 (m, 1H), 2.85-2.18 (m, 2H), 2.15-1.48 (m, 5H), 1.43-1.33 (m, 3H), 1.08-0.88 (m, 3H), 0.82-0.57 (m, 3H) ppm. MS: M/e 459 (M+1)⁺.

### Compound A40: 2-(5-(1-((2R,5S)-4-(2-(cyanomethyl)-5-methyl-6-oxo-5,6-dihydroimidazo[1,2-b]pyridazin-8-yl)-2,5-diethylpiperazin-1-yl)ethyl)pyridin-2-yl)-2-methylpropanenitrile

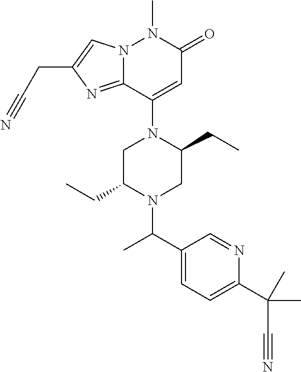 [SMILES: CC[C@H]1CN(C(C)c2ccc(C(C)(C)C#N)nc2)[C@H](CC)CN1c1cc(=O)n(C)n2cc(CC#N)nc12]

### Step A: 2-(5-bromopyridin-2-yl)-2-methylpropanenitrile

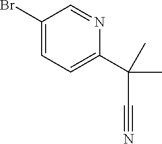

To a mixture of 2-(5-bromopyridin-2-yl)acetonitrile (2.0 g, 10 mmol) and t-BuOK (2.8 g, 25 mmol) in THF (30 mL) was added Mel (3.6 g, 25 mmol) in drops at 0° C. and the mixture was stirred at RT for 16 hours. The resulted suspension was diluted with EtOAc (50 mL), filtered. The filtrate was washed with brine (20 mL×3), dried over Na₂SO₄, filtered and concentrated under reduced pressure. The resulting residue was purified by column chromatography to give the titled compound (2.05 g, 91%). ¹H NMR (400 MHz, CDCl₃) δ 8.65 (s, 1H), 7.85 (d, J=8.4 Hz, 1H), 7.50 (d, J=8.4 Hz, 1H), 1.74 (s, 6H) ppm.

### Step B: 2-(5-acetylpyridin-2-yl)-2-methylpropanenitrile

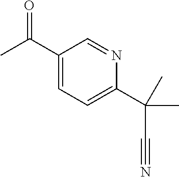

A mixture of 2-(5-bromopyridin-2-yl)acetonitrile (1.0 g, 4.4 mmol), tributyl(1-ethoxyvinyl)stannane (2.0 g, 5.5 mmol) and Pd(PPh₃)₂Cl₂ (160 mg, 0.23 mmol) in toluene (12 mL) was stirred at 100° C. under N₂ for 16 hrs. The mixture was cooled and diluted with EtOAc (30 mL). The suspension was filtered through a celite pad and the filtrate was washed with brine (20 mL×2), dried over Na₂SO₄, filtered and the filtrate was concentrated. The resulted residue was diluted with EtOAc (20 mL) and added a solution of HCl/Dioxane (4M, 3 mL) and stirred for 20 minutes. The resulted mixture was basified with NaHCO₃ aqueous solution and the mixture was extracted with EtOAc (20 mL×3). The extracts were combined and washed with brine (10 mL×2), dried over Na₂SO₄, filtered and concentrated to dryness. The resulted residue was purified by column chromatography to give the titled compound (710 mg, 86%). ¹H NMR (400 MHz, CDCl₃) δ 9.14 (s, 1H), 8.27 (d, J=8.4 Hz, 1H), 7.71 (d, J=8.4 Hz, 1H), 2.64 (s, 3H), 1.78 (s, 6H) ppm.

### Step C: 2-(5-(1-hydroxyethyl)pyridin-2-y)-2-methylpropanenitrile

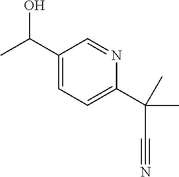

To a solution of 2-(5-acetylpyridin-2-yl)-2-methylpropanenitrile (700 mg, 3.7 mmol) in MeOH (10 mL) was added NaBH₄ (140 mg, 3.7 mmol) in portions at RT and the mixture was stirred at RT for 1 hour. The mixture was diluted with EtOAc (20 mL), treated with NaHCO₃ (20 mL). The aqueous layer was extracted with EtOAc (10 mL×2). The combined organics was washed with brine (20 mL×2), dried over Na₂SO₄, filtered and concentrated to dryness to give the titled compound (660 mg, 94%). ¹H NMR (400 MHz, DMSO-d₆) δ 8.54 (s, 1H), 7.81 (d, J=8.0 Hz, 1H), 7.53 (d, J=8.0 Hz, 1H), 5.33 (d, J=4.4 Hz, 1H), 4.87-4.68 (m, 1H), 1.67 (s, 6H), 1.34 (d, J=6.4 Hz, 3H) ppm.

### Step D: 2-(5-(1-((2R,5S)-4-(2-(cyanomethyl)-5-methyl-6-oxo-5,6-dihydroimidazo[1,2-b]pyridazin-8-yl)-2,5-diethylpiperazin-1-yl)ethyl)pyridin-2-yl)-2-methylpropanenitrile

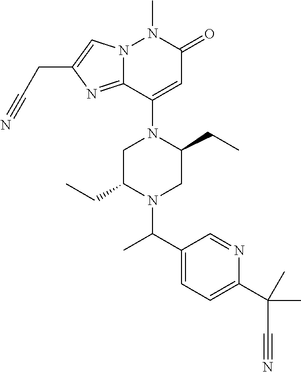 [SMILES: CC[C@H]1CN(C(C)c2ccc(C(C)(C)C#N)nc2)[C@H](CC)CN1c1cc(=O)n(C)n2cc(CC#N)nc12]

To a solution of 2-(8-((2S,5R)-2,5-diethylpiperazin-1-yl)-5-methyl-6-oxo-5,6-dihydroimidazo[1,2-b]pyridazin-2-yl)acetonitrile (33 mg, 0.1 mmol), 2-(5-(1-hydroxyethyl)pyridin-2-yl)-2-methylpropanenitrile (38 mg, 0.2 mmol) and (cyanomethyl)trimethylphosphonium iodide (150 mg, 0.6 mmol) in CH₃CN (0.5 mL) was added DIPEA (77 mg, 0.60 mmol). The mixture was stirred at 105° C. in a sealed tube for 16 hours. The mixture was cooled and diluted with EtOAc (10 mL), washed with brine (5 mL×3), dried over Na₂SO₄, filtered and concentrated under reduced pressure. The resulting residue was purified by column chromatography to give the titled compound (42 mg, 84%). ¹H NMR (400 MHz, CD₃OD) δ 8.64-8.51 (m, 1H), 7.96-7.85 (m, 1H), 7.78 (s, 1H), 7.66-7.56 (m, 1H), 5.67-5.54 (m, 1H), 4.00-3.91 (m, 2H), 3.90-3.62 (m, 4H), 3.58-3.32 (m, 2H), 3.29-3.12 (m, 1H), 3.04-2.85 (m, 1H), 2.79-2.34 (m, 1H), 2.32-1.99 (m, 1H), 1.94-1.52 (m, 10H), 1.42-1.32 (m, 3H), 1.10-0.88 (m, 3H), 0.81-0.51 (m, 3H) ppm. MS: M/e 501 (M+1)⁺.

### Compound A41: 2-(8-((2S,5R)-2,5-diethyl-4-(1-(6-(2-hydroxypropan-2-yl)pyridin-3-yl)ethyl)piperazin-1-yl)-5-methyl-6-oxo-5,6-dihydroimidazo[1,2-b]pyridazin-2-yl)acetonitrile

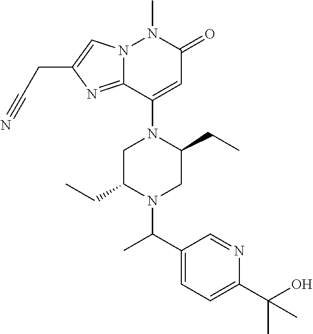 [SMILES: CC[C@H]1CN(C(C)c2ccc(C(C)(C)O)nc2)[C@H](CC)CN1c1cc(=O)n(C)n2cc(CC#N)nc12]

### Step A: 2-(5-bromopyridin-2-yl)propan-2-ol

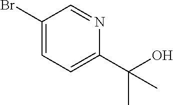

To a 0° C. solution of 1-(5-bromopyridin-2-yl)ethan-1-one (5.0 g, 25 mmol) in THF (100 mL) was added MeMgBr (3.0 M, 10 mL, 30 mmol) in drops under N₂. The mixture was stirred at room temperature for 2 hours. Aqueous solution of NH₄Cl (100 mL) was added and the mixture was extracted with EtOAc (50 mL×2), the organics were combined and washed with brine (50 mL×2), dried over Na₂SO₄, filtered and concentrated. The resulting residue was purified by column chromatography to give the titled compound (1.1 g, 20%). ¹HNMR (400 MHz, DMSO-d₆) δ 8.57 (s, 1H), 7.98 (d, J=8.4 Hz, 1H), 7.60 (d, J=8.4 Hz, 1H), 5.29 (s, 1H), 1.40 (s, 6H).

### Step B: 1-(6-(2-hydroxypropan-2-yl)pyridin-3-yl)ethan-1-one

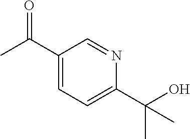

A mixture of 2-(5-bromopyridin-2-yl)propan-2-ol (540 mg, 2.5 mmol), tributyl(1-ethoxyvinyl)stannane (1.2 g, 3.3 mmol) and Pd(PPh₃)₂Cl₂ (90 mg, 0.13 mmol) in toluene (6 mL) was stirred at 100° C. under N₂ for 16 hrs. The mixture was cooled and diluted with EtOAc (20 mL). The suspension was filtered through a celite pad and the filtrate was washed with brine (20 mL×3), dried over Na₂SO₄, filtered and the filtrate was concentrated. The resulted residue was diluted with EtOAc (10 mL) and added a solution of HCl/Dioxane (4M, 2 mL) and stirred for 20 minutes. The resulted mixture was basified with NaHCO₃ aqueous solution and the mixture was extracted with EtOAc (10 mL×3). The extracts were combined and washed with brine (10 mL×2), dried over Na₂SO₄, filtered and concentrated to dryness. The resulted residue was purified by column chromatography to give the titled compound (265 mg, 59%). ¹H NMR (400 MHz, DMSO-d₆) δ 9.04 (s, 1H), 8.26 (d, J=8.4 Hz, 1H), 7.80 (d, J=8.4 Hz, 1H), 5.39 (s, 1H), 2.61 (s, 3H), 1.45 (s, 6H).

### Step C: 2-(5-(1-hydroxyethyl)pyridin-2-yl)propan-2-ol

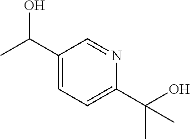

To a solution of 1-(6-(2-hydroxypropan-2-yl)pyridin-3-yl)ethan-1-one (260 mg, 1.45 mmol) in MeOH (4 mL) was added NaBH₄ (55 mg, 1.45 mmol) in portions at RT and the mixture was stirred at RT for 1 hour. The mixture was diluted with EtOAc (20 mL), treated with NaHCO₃ (20 mL). The aqueous layer was extracted with EtOAc (10 mL×2). The combined organics was washed with brine (20 mL×2), dried over Na₂SO₄, filtered and concentrated to dryness to give the titled compound (220 mg, 84%). ¹H NMR (400 MHz, DMSO-d₆) δ 8.41 (s, 1H), 7.68 (d, J=8.0 Hz, 1H), 7.57 (d, J=8.0 Hz, 1H), 5.22 (d, J=4.0 Hz, 1H), 5.14 (s, 1H), 4.80-4.62 (m, 1H), 1.40 (s, 6H), 1.33 (d, J=6.4 Hz, 3H).

### Step D: 2-(8-((2S,5R)-2,5-diethyl-4-(1-(6-(2-hydroxypropan-2-yl)pyridin-3-yl)ethyl)piperazin-1-yl)-5-methyl-6-oxo-5,6-dihydroimidazo[1,2-b]pyridazin-2-yl)acetonitrile

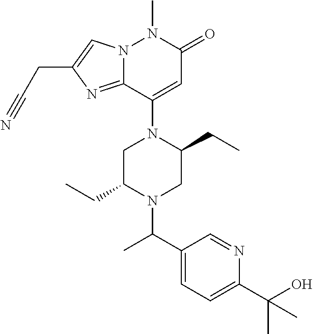 [SMILES: CC[C@H]1CN(C(C)c2ccc(C(C)(C)O)nc2)[C@H](CC)CN1c1cc(=O)n(C)n2cc(CC#N)nc12]

To a solution of 2-(8-((2S,5R)-2,5-diethylpiperazin-1-yl)-5-methyl-6-oxo-5,6-dihydroimidazo[1,2-b]pyridazin-2-yl)acetonitrile (33 mg, 0.1 mmol), 2-(5-(1-hydroxyethyl)pyridin-2-yl)propan-2-ol (36 mg, 0.2 mmol) and (cyanomethyl)trimethylphosphonium iodide (150 mg, 0.6 mmol) in CH₃CN (0.5 mL) was added DIPEA (129 mg, 1.0 mmol). The mixture was stirred at 105° C. in a sealed tube for 16 hours. The mixture was cooled and diluted with EtOAc (10 mL), washed with brine (5 mL×3), dried over Na₂SO₄, filtered and concentrated under reduced pressure. The resulting residue was purified by column chromatography to give the titled compound (32 mg, 76%). ¹H NMR (400 MHz, CD₃OD) δ 8.54-8.40 (m, 1H), 7.90-7.73 (m, 2H), 7.71-7.59 (m, 1H), 5.67-5.54 (m, 1H), 4.00-3.89 (m, 2H), 3.88-3.60 (m, 4H), 3.55-3.42 (m, 1H), 3.35-3.10 (m, 2H), 3.07-2.83 (m, 1H), 2.75-2.35 (m, 1H), 2.34-2.00 (m, 1H), 1.98-1.49 (m, 10H), 1.42-1.33 (m, 3H), 1.08-0.89 (m, 3H), 0.76-0.55 (m, 3H). MS: M/e 492 (M+1)⁺.

### Compound A42: 2-(8-((2S,5R)-4-(1-(6-cyclopropyl-2-fluoropyridin-3-yl)ethyl)-2,5-diethylpiperazin-1-yl)-5-methyl-6-oxo-5,6-dihydroimidazo[1,2-b]pyridazin-2-yl)acetonitrile

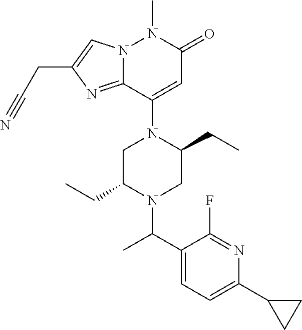 [SMILES: CC[C@H]1CN(C(C)c2ccc(C3CC3)nc2F)[C@H](CC)CN1c1cc(=O)n(C)n2cc(CC#N)nc12]

### Step A: 6-cyclopropyl-2-fluoronicotinaldehyde

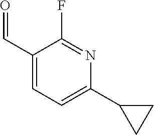 [SMILES: O=Cc1ccc(C2CC2)nc1F]

To a solution of 6-chloro-2-fluoronicotinaldehyde (0.32 g, 2 mmol) and cyclopropylboronic acid (258 mg, 3 mmol) in toluene/H₂O (10 mL/1 mL) were added dichlorobis(tricyclohexylphosphine)palladium(II) (147 mg, 0.2 mmol) and K₃PO₄ (842 mg, 4 mmol). The reaction mixture was stirred at 100° C. under N₂ overnight. The mixture was cooled to room temperature, diluted with water (40 mL), extracted with EA (60 mL×2), washed with brine, dried over Na₂SO₄, filtered, and concentrated to dryness. The residue was purified by flash column chromatography to give the titled compound (130 mg, 39%). MS: M/e 166 (M+1)⁺.

### Step B: 1-(6-cyclopropyl-2-fluoropyridin-3-yl)ethan-1-ol

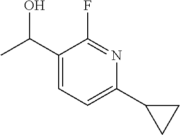

To a solution of 6-cyclopropyl-2-fluoronicotinaldehyde (1.3 g, 7.8 mmol) in THF (50 mL) was added a solution of CH₃MgBr in ether (3 mL, 9 mmol) slowly at 0° C. The resulting mixture was stirred at room temperature for 1 hour. The reaction mixture was quenched with saturated NH4Cl (30 mL) and H₂O (100 mL) and extracted with EtOAc (100 mL×2), washed with brine, dried over Na₂SO₄, filtered, and concentrated to dryness. The residue was purified by flash column chromatography to give the titled compound (900 mg, 63%). ¹H NMR (400 MHz, DMSO-d₆) δ 7.84-7.76 (m, 1H), 7.10 (d, J=7.2 Hz, 1H), 5.31 (d, J=4.0 Hz, 1H), 4.85-4.74 (m, 1H), 2.08-1.98 (m, 1H), 1.27 (d, J=6.4 Hz, 3H), 0.96-0.87 (m, 2H), 0.84-0.75 (m, 2H) ppm.

### Step C: 2-(8-((2S,5R)-4-(1-(6-cyclopropyl-2-fluoropyridin-3-yl)ethyl)-2,5-diethylpiperazin-1-yl)-5-methyl-6-oxo-5,6-dihydroimidazo[1,2-b]pyridazin-2-yl)acetonitrile

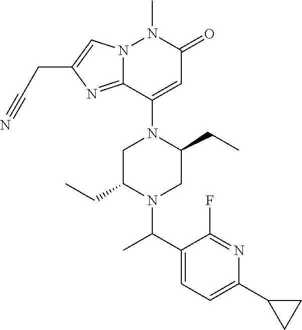 [SMILES: CC[C@H]1CN(C(C)c2ccc(C3CC3)nc2F)[C@H](CC)CN1c1cc(=O)n(C)n2cc(CC#N)nc12]

To a solution of 1-(6-cyclopropyl-2-fluoropyridin-3-yl)ethan-1-ol (181 mg, 1 mmol), 2-(8-((2S,5R)-2,5-diethylpiperazin-1-yl)-5-methyl-6-oxo-5,6-dihydroimidazo[1,2-b]pyridazin-2-yl)acetonitrile (164 mg, 0.5 mmol) and (cyanomethyl)trimethyl phosphonium iodide (364 mg, 1.5 mmol) in CH₃CN (4 mL) was added DIPEA (322 mg, 2.5 mmol). The mixture was sealed in a bottle and heated at 100° C. for 16 hours. The mixture was cooled to room temperature, diluted with water, extracted with EA (80 mL×2), washed with brine, dried over Na₂SO₄, filtered, and concentrated to dryness. The resulting residue was purified by flash column chromatography (DCM/MeOH=20/1) to give the titled Compound A42, which was further separated into CompoundA42a (43 mg) and CompoundA42b (63 mg) by Prep-HPLC(Method A).

CompoundA42a (the earlier peak): ¹H NMR (400 MHz, CD₃OD) δ 7.86 (t, J=7.6 Hz, 1H), 7.78 (s, 1H), 7.17 (d, J=7.2 Hz, 1H), 5.60 (s, 1H), 4.10-4.01 (m, 1H), 3.93 (s, 2H), 3.73 (s, 3H), 3.35-3.31 (m, 2H), 3.28-3.22 (m, 1H), 2.94-2.86 (m, 2H), 2.48-2.32 (m, 1H), 2.10-1.95 (m, 2H), 1.86-1.70 (m, 1H), 1.58-1.44 (m, 2H), 1.35 (d, J=5.6 Hz, 3H), 1.05-0.85 (m, 7H), 0.77 (t, J=7.2 Hz, 3H) ppm. MS: M/e 492 (M+1)⁺.

CompoundA42b (the later peak):¹H NMR (400 MHz, CD₃OD) δ 7.93 (t, J=8.0 Hz, 1H), 7.78 (s, 1H), 7.15 (d, J=8.0 Hz, 1H), 5.60 (s, 1H), 3.93 (s, 2H), 3.92-3.83 (m, 1H), 3.73 (s, 3H), 3.53-3.42 (m, 1H), 3.35-3.31 (m, 2H), 3.15-3.05 (m, 1H), 2.75-2.62 (m, 1H), 2.37-2.28 (m, 1H), 2.08-1.85 (m, 2H), 1.76-1.42 (m, 3H), 1.30 (d, J=6.8 Hz, 3H), 1.07-0.85 (m, 7H), 0.67 (t, J=7.6 Hz, 3H) ppm. MS: M/e 492 (M+1)⁺.

Compound A43: 2-(8-((2S,5R)-4-(1-(6-cyclopropylpyridin-3-yl)ethyl)-5-ethyl-2-methylpiperazin-1-yl)-5-methyl-6-oxo-5,6-dihydroimidazo[1,2-b]pyridazin-2-yl)acetonitrile

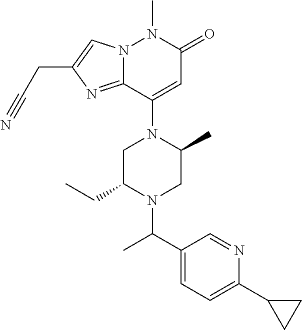 [SMILES: CC[C@@H]1CN(c2cc(=O)n(C)n3cc(CC#N)nc23)[C@@H](C)CN1C(C)c1ccc(C2CC2)nc1]

A mixture of 2-(8-((2S,5R)-5-ethyl-2-methylpiperazin-1-yl)-5-methyl-6-oxo-5,6-dihydroimidazo[1,2-b]pyridazin-2-yl)acetonitrile (150 mg, 0.48 mmol), 1-(6-cyclopropylpyridin-3-yl)ethan-1-ol (156 mg, 0.96 mmol), (cyanomethyl)trimethylphosphonium iodide (350 mg, 1.44 mmol) and DIPEA (310 mg, 2.4 mmol) in CH₃CN (5 mL) was stirred overnight at 100° C. in a sealed tube. The reaction mixture was diluted with EtOAc (20 mL), washed with H₂O, brine, dried over Na₂SO₄, concentrated to dryness. The resulting residue was purified by flash column chromatography to give the crude titled Compound A43, which was further separated into Compound A43a (25 mg) and Compound A43b (27 mg) by Prep-HPLC (Method B).

Compound A43a (the earlier peak): ¹H NMR (400 MHz, CD₃OD) δ 8.30 (s, 1H), 7.77 (s, 1H), 7.71 (d, J=7.2 Hz, 1H), 7.21 (d, J=8.0 Hz, 1H), 5.62 (s, 1H), 5.25-5.00 (m, 1H), 4.86-4.80 (m, 1H), 3.92 (s, 2H), 3.78 (q, J=6.4 Hz, 1H), 3.73 (s, 3H), 3.29-3.24 (m, 1H), 3.00-2.76 (m, 2H), 2.42-2.33 (m, 1H), 2.14-2.03 (m, 1H), 1.62-1.46 (m, 2H), 1.40-1.32 (m, 6H), 1.08-0.91 (m, 4H), 0.77 (t, J=7.2 Hz, 3H) ppm. MS: M/e 460 (M+1)⁺.

Compound A43b (the later peak): ¹H NMR (400 MHz, CD₃OD) δ 8.33 (s, 1H), 7.78 (s, 1H), 7.73 (d, J=8.0 Hz, 1H), 7.17 (d, J=8.0 Hz, 1H), 5.61 (s, 1H), 5.50-5.25 (m, 1H), 4.50-4.48 (m, 1H), 3.93 (s, 2H), 3.73 (s, 3H), 3.65 (q, J=6.4 Hz, 1H), 3.53-3.45 (m, 1H), 3.16-3.08 (m, 1H), 2.76-2.69 (m, 1H), 2.25-2.18 (m, 1H), 2.12-2.03 (m, 1H), 1.72-1.48 (m, 2H), 1.32 (d, J=6.4 Hz, 3H), 1.20 (d, J=6.4 Hz, 3H), 1.10-0.89 (m, 7H) ppm. MS: M/e 460(M+1)⁺.

Compound A44:-2-(8-((2S,5R)-2,5-diethyl-4-(1-(6-methoxypyridin-3-yl)ethyl)piperazin-1-yl)-5-methyl-6-oxo-5,6-dihydroimidazo[1,2-b]pyridazin-2-yl)acetonitrile

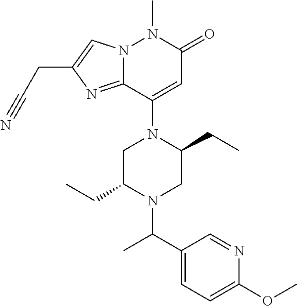 [SMILES: CC[C@H]1CN(C(C)c2ccc(OC)nc2)[C@H](CC)CN1c1cc(=O)n(C)n2cc(CC#N)nc12]

### Step A: 1-(6-methoxypyridin-3-yl)ethan-1-ol

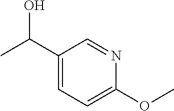

To a solution of 1-(6-methoxypyridin-3-yl)ethan-1-one (302 mg, 2 mmol) in MeOH (5 mL) was added NaBH₄ (61 mg, 1.6 mmol) at room temperature and the mixture was stirred at room temperature for 30 minutes. The resulting mixture was treated with water (50 ml), extracted with DCM (10 mL×2). The combined organic layers were dried over Na₂SO₄ and concentrated to dryness. The resulting residue (250 mg, crude) was used in the next step. MS: M/e 154 (M+1)⁺.

### Step B: 2-(8-((2S,5R)-2,5-diethyl-4-(1-(6-methoxypyridin-3-yl)ethyl)piperazin-1-yl)-5-methyl-6-oxo-5,6-dihydroimidazo[1,2-b]pyridazin-2-yl)acetonitrile

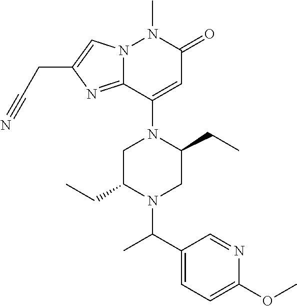 [SMILES: CC[C@H]1CN(C(C)c2ccc(OC)nc2)[C@H](CC)CN1c1cc(=O)n(C)n2cc(CC#N)nc12]

A mixture of 1-(6-methoxypyridin-3-yl)ethan-1-ol (186 mg, 1.22 mmol), 2-(8-((2S,5R)-2,5-diethylpiperazin-1-yl)-5-methyl-6-oxo-5,6-dihydroimidazo[1,2-b]pyridazin-2-yl) acetonitrile (200 mg, 0.61 mmol), (cyanomethyl)trimethylphosphonium iodide (590 mg, 2.44 mmol) and DIPEA (314 mg, 2.44 mmol) in MeCN (4 mL) was stirred at 100° C. overnight. The reaction mixture was diluted with water (50 mL), extracted with water (10 mL×2), dried over Na₂SO₄ and concentrated to dryness. The resulting residue was purified by flash column chromatography (0-10% MeOH in DCM in 30 minutes) to give the titled Compound A44 (200 mg, crude) which was further separated into Compound A44a (16 mg) and Compound A44b (19 mg) by Prep-HPLC (Method B).

Compound A44a (the earlier peak) ¹H NMR (400 MHz, DMSO-d₆) δ 8.07 (s, 1H), 7.83 (s, 1H), 7.68 (d, J=8.2 Hz, 1H), 6.80 (d, J=8.4 Hz, 1H), 5.49 (s, 1H), 4.00 (s, 2H), 3.82 (s, 3H), 3.69 (d, J=6.0 Hz, 1H), 3.58 (s, 3H), 3.32-3.25 (m, 2H), 3.10 (d, J=12.2 Hz, 1H), 2.85 (d, J=12.1 Hz, 1H), 2.74 (d, J=9.8 Hz, 1H), 2.26 (s, 1H), 1.93 (dd, J=14.7, 7.1 Hz, 1H), 1.64 (d, J=7.4 Hz, 1H), 1.41 (s, 2H), 1.25-1.23 (m, 3H), 0.83 (s, 3H), 0.63 (s, 3H) ppm. MS: M/e 464 (M+1)⁺.

Compound A44b (the later peak) ¹H NMR (400 MHz, DMSO-d₆) δ 8.07 (s, 1H), 7.84 (s, 1H), 7.68 (dd, J=8.5, 1.8 Hz, 1H), 6.78 (d, J=8.5 Hz, 1H), 5.48 (s, 1H), 4.01 (s, 2H), 3.81 (s, 3H), 3.63-3.52 (m, 4H), 3.30-3.26 (m, 2H), 3.01 (d, J=9.1 Hz, 1H), 2.53-2.49 (m, 2H), 2.21 (d, J=12.1 Hz, 1H), 1.76 (dd, J=14.0, 6.9 Hz, 1H), 1.46 (dd, J=19.7, 7.6 Hz, 3H), 1.23 (t, J=11.1 Hz, 3H), 0.93 (t, J=6.9 Hz, 3H), 0.53 (t, J=6.8 Hz, 3H) ppm. MS: M/e 464 (M+1)⁺.

Compound A45: 2-(8-((2S,5R)-2,5-diethyl-4-(1-(6-isopropoxypyridin-3-yl)ethyl)piperazin-1-yl)-5-methyl-6-oxo-5,6-dihydroimidazo[1,2-b]pyridazin-2-yl)acetonitrile

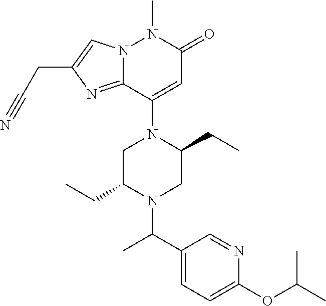 [SMILES: CC[C@H]1CN(C(C)c2ccc(OC(C)C)nc2)[C@H](CC)CN1c1cc(=O)n(C)n2cc(CC#N)nc12]

### Step A: 1-(6-isopropoxypyridin-3-yl)ethan-1-one

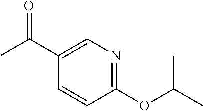

A mixture of 1-(6-fluoropyridin-3-yl)ethan-1-one (695 mg, 5 mmol), propan-2-ol (600 mg, 10 mmol) and potassium tert-butoxide (1.16 g, 10 mmol) in DMSO (10 mL) was stirred at 50° C. for 16 hours. The mixture was treated with water (50 ml), extracted with EtOAc (20 ml×2), washed with brine (20 mL), dried and concentrated to dryness. The resulting residue was purified by flash column chromatography (0-50% EtOAc in PE in 20 minutes) to give the titled compound (290 mg, 32%). MS: M/e 180 (M+1)⁺.

### Step B: 1-(6-isopropoxypyridin 3-yl)ethan-1-ol

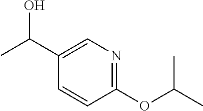

To a solution of 1-(6-isopropoxypyridin-3-yl)ethan-1-one (280 mg, 1.56 mmol) in MeOH (5 mL) was added NaBH₄ (47 mg, 1.25 mmol) at room temperature and the mixture was stirred at room temperature for 30 minutes. The resulting mixture was treated with water (20 ml), extracted with EtOAc (10 mL×2). The combined organic layers were dried over Na₂SO₄ and concentrated to dryness. The resulting residue (240 mg, crude) was used in the next step directly. MS: M/e 182 (M+1)⁺.

### Step C: 2-(8-((2S,5R)-2,5-diethyl-4-(1-(6-isopropoxypyridin-3-yl)ethyl)piperazin-1-yl)-5-methyl-6-oxo-5,6-dihydroimidazo[1,2-b]pyridazin-2-yl)acetonitrile

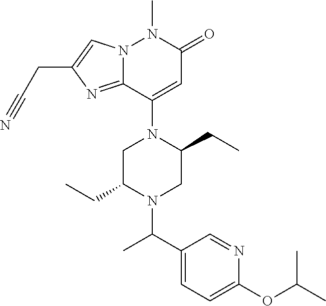 [SMILES: CC[C@H]1CN(C(C)c2ccc(OC(C)C)nc2)[C@H](CC)CN1c1cc(=O)n(C)n2cc(CC#N)nc12]

A mixture of 1-(6-isopropoxypyridin-3-yl)ethan-1-ol(221 mg, 1.22 mmol), 2-(8-((2S,5R)-2,5-diethylpiperazin-1-yl)-5-methyl-6-oxo-5,6-dihydroimidazo[1,2-b]pyridazin-2-yl) acetonitrile (200 mg, 0.61 mmol), (cyanomethyl)trimethylphosphonium iodide (590 mg, 2.44 mmol) and DIPEA (314 mg, 2.44 mmol) in MeCN (5 mL) was stirred at 100° C. overnight. The reaction mixture was diluted with water (40 mL), extracted with EtOAc (10 mL×3), dried over Na₂SO₄ and concentrated to dryness. The resulting residue was purified by flash column chromatography (0-10% MeOH in DCM in 30 minutes) to give the titled Compound A45 (250 mg, crude), which was further separated into Compound A45a (21 mg) and Compound A45b (19 mg) by Prep-HPLC(Method A).

Compound A45a (the earlier peak):¹H NMR (400 MHz, DMSO-d₆) δ 8.04 (d, J=2.0 Hz, 1H), 7.84 (s, 1H), 7.65 (dd, J=8.5, 2.3 Hz, 1H), 6.72 (d, J=8.5 Hz, 1H), 5.49 (s, 1H), 5.21 (dt, J=12.3, 6.2 Hz, 1H), 4.01 (s, 2H), 3.68 (q, J=6.2 Hz, 1H), 3.59 (s, 3H), 3.32-3.29 (m, 2H), 3.11 (d, J=10.8 Hz, 1H), 2.84 (d, J=11.8 Hz, 1H), 2.74 (dd, J=11.9, 3.4 Hz, 1H), 2.33-2.21 (m, 1H), 2.00-1.86 (m, 1H), 1.71-1.56 (m, 1H), 1.39 (dd, J=18.7, 10.7 Hz, 2H), 1.27 (d, J=6.1 Hz, 9H), 0.83 (t, J=7.1 Hz, 3H), 0.64 (t, J=7.1 Hz, 3H) ppm. MS: M/e 492 (M+1)⁺.

Compound A45b (the later peak): ¹H NMR (400 MHz, DMSO-d₆) δ 8.05 (d, J=1.8 Hz, 1H), 7.84 (s, 1H), 7.65 (dd, J=8.5, 2.1 Hz, 1H), 6.69 (d, J=8.5 Hz, 1H), 5.49 (s, 1H), 5.20 (dt, J=12.3, 6.1 Hz, 1H), 4.01 (s, 2H), 3.64-3.50 (m, 4H), 3.32-3.28 (m, 3H), 3.01 (d, J=9.3 Hz, 1H), 2.51-2.49 (m, 1H), 2.22 (d, J=11.9 Hz, 1H), 1.78 (dd, J=13.8, 7.2 Hz, 1H), 1.60-1.34 (m, 3H), 1.31-1.17 (m, 9H), 0.94 (t, J=6.9 Hz, 3H), 0.54 (d, J=6.5 Hz, 3H) ppm. MS: M/e 492 (M+1)⁺.

Compound A46: 2-(8-((2S,5R)-2,5-diethyl-4-(1-(6-(2-fluoropropan-2-yl)pyridin-3-yl)ethyl)piperazin-1-yl)-5-methyl-6-oxo-5,6-dihydroimidazo[1,2-b]pyridazin-2-yl)acetonitrile

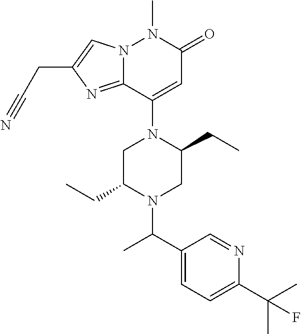 [SMILES: CC[C@H]1CN(C(C)c2ccc(C(C)(C)F)nc2)[C@H](CC)CN1c1cc(=O)n(C)n2cc(CC#N)nc12]

### Step A: methyl 6-acetylnicotinate

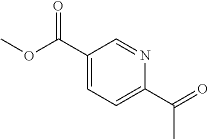

A mixture of methyl 6-chloronicotinate (3.43 g, 20 mmol), tributyl(1-ethoxyvinyl)stannane (10.8 g, 30 mmol) and Pd(PPh₃)Cl₂ (1.4 g, 2 mmol) in toluene (80 mL) was stirred overnight under N₂ at 100° C. The reaction mixture was allowed cool to R.T. and washed with aq.HCl (2.0 M, 20 mL) for 30 min. The organic layer was separated and the aqueous layer was extracted with EtOAc (20 mL×2). The combined organic layers were washed with brine, dried over Na₂SO₄, concentrated and purified by flash column chromatography to give the titled compound (1.2 g, 33.5%) as a white solid. MS: M/e 180 (M+1)⁺.

### Step B: methyl 6-(2-hydroxypropan-2-yl)nicotinate

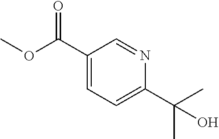

To a stirred solution of methyl 6-acetylnicotinate (358 mg, 2 mmol) in THF (20 mL) was added dropwise MeMgBr (3.0 M, 1.33 mL, 4 mmol) at 0° C. After the addition, the reaction mixture was stirred for 20 min. The reaction was quenched with aq.NH₄Cl, extracted with EtOAc (20 mL×3). The combined organic layers were washed with brine, dried over Na₂SO₄, concentrated and purified by flash column chromatography to give the titled compound (280 mg, 57%) as colorless oil. MS: M/e 196 (M+1)⁺.

### Step C: methyl 6-(2-fluoropropan-2-yl)nicotinate

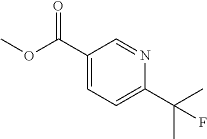

To a stirred solution of methyl 6-(2-hydroxypropan-2-yl)nicotinate (273 mg, 1.4 mmol) in CH₂Cl₂ (10 mL) was added dropwise a solution of DAST (451 mg, 2.8 mmol) in CH₂Cl₂ (3 mL) at 0° C. After the addition, the reaction was stirred for 3 hours. The reaction was quenched with aq.NaHCO₃, then extracted with CH₂Cl₂ (10 mL×2). The combined organic layers were washed with brine, dried over Na₂SO₄, concentrated and purified by flash column chromatography to give the titled compound (225 mg, 81.6%) as colorless oil. MS: M/e 198 (M+1)⁺.

### Step D: 6-(2-fluoropropan-2-yl)nicotinic acid

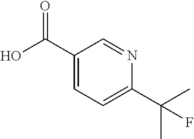

To a stirred solution of methyl 6-(2-fluoropropan-2-yl)nicotinate (225 mg, 1.14 mmol) in MeOH (5 mL) was added aq.NaOH (2.0 M, 2 mL). After then, the mixture was stirred overnight. The reaction mixture was acidified to pH=5-6 with aq.HCl, then concentrated to give the crude product, which was directly used to the next step. MS: M/e 184 (M+1)⁺.

### Step E: 6-(2-fluoropropan-2-yl)-N-methoxy-N-methylnicotinamide

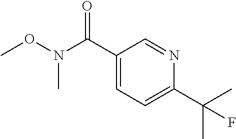 [SMILES: CON(C)C(=O)c1ccc(C(C)(C)F)nc1]

To a stirred solution of 6-(2-fluoropropan-2-yl)nicotinic acid (crude, 1.14 mmol), N,O-dimethylhydroxylamine hydrochloride (133 mg, 1.37 mmol), HATU (523 mg, 1.37 mmol) and DIPEA (294 mg, 2.28 mmol) was stirred overnight. The reaction mixture was washed with H₂O, extracted with CH₂Cl₂ (15 mL×2). The combined organic layers were washed with brine, dried over Na₂SO₄, concentrated and purified by flash column chromatography to give the titled compound (223 mg, 86.5%) as colorless oil. MS: M/e 227 (M+1)⁺.

### Step F: 1-(6-(2-fluoropropan-2-yl)pyridin-3-yl)ethan-1-one

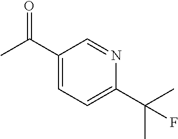

To a stirred solution of 6-(2-fluoropropan-2-yl)-N-methoxy-N-methylnicotinamide (223 mg, 0.986 mmol) in THF (10 mL) was added dropwise MeMgBr (3.0 M, 0.5 mL, 1.45 mmol) at 0° C. After then, the mixture was stirred for 30 min. The reaction was quenched with aq.NH₄Cl, extracted with EtOAc (15 mL×3). The combined organic layers were washed with brine, dried over Na₂SO₄, concentrated and purified by flash column chromatography to give the titled compound (120 mg, 67.2%) as colorless oil. MS: M/e 182 (M+1)⁺.

### Step G: 1-(6-(2-fluoropropan-2-yl)pyridin-3-yl)ethan-1-ol

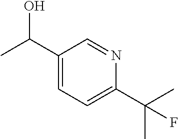

To a stirred solution of 1-(5-methoxypyridin-2-yl)ethan-1-one (120 mg, 0.663 mmol) in MeOH (5 mL) was added NaBH₄ (25.2 mg, 0.663 mmol). After then, the reaction mixture was stirred for 10 min. The reaction mixture was quenched with H₂O, then most MeOH was removed to give the aqueous layer, then extracted with EtOAc (10 mL×2). The combined organic layers were washed with brine, dried over Na₂SO₄, concentrated to give the titled compound (120 mg, 99%) as colorless oil. MS: M/e 184 (M+1)⁺.

### Step H: 2-(8-((2S,5R)-2,5-diethyl-4-(1-(6-(2-fluoropropan-2-yl)pyridin-3-yl)ethyl)piperazin-1-yl)-5-methyl-6-oxo-5,6-dihydroimidazo[1,2-b]pyridazin-2-yl)acetonitrile

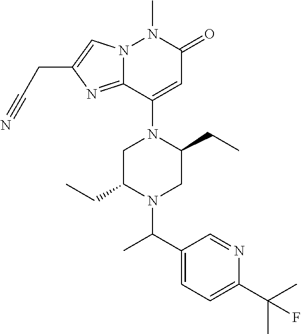 [SMILES: CC[C@H]1CN(C(C)c2ccc(C(C)(C)F)nc2)[C@H](CC)CN1c1cc(=O)n(C)n2cc(CC#N)nc12]

A mixture of 2-(8-((2S,5R)-2,5-diethylpiperazin-1-yl)-5-methyl-6-oxo-5,6-dihydroimidazo[1,2-b]pyridazin-2-yl)acetonitrile (100 mg, 0.3 mmol), 1-(4-(2-fluoropropan-2-yl)phenyl)ethan-1-ol (111.5 mg, 0.6 mmol), (cyanomethyl)trimethylphosphonium iodide (219 mg, 0.9 mmol) and DIPEA (192 mg, 1.5 mmol) in CH₃CN (8 mL) was stirred overnight at 100° C. in a sealed tube. The reaction mixture was diluted with EtOAc (20 mL), washed with H₂O, brine, dried over Na₂SO₄, concentrated to dryness. The resulting residue was purified by flash column chromatography to give titled Compound A46, which was further separated into Compound A46a (28 mg) and Compound A46b (32 mg) by Prep-HPLC (Method B).

Compound A46a (the earlier peak): ¹H NMR (400 MHz, CD₃OD) δ 8.48 (s, 1H), 7.88 (d, J=8.0 Hz, 1H), 7.78 (s, 1H), 7.60 (d, J=8.0 Hz, 1H), 5.61 (s, 1H), 3.93 (s, 2H), 3.87 (q, J=6.4 Hz, 1H), 3.73 (s, 3H), 3.33-3.31 (m, 2H), 3.30-3.26 (m, 1H), 3.05-2.86 (m, 2H), 2.44-2.33 (m, 1H), 2.16-2.01 (m, 1H), 1.90-1.76 (m, 1H), 1.72 (s, 3H), 1.66 (s, 3H), 1.60-1.49 (m, 2H), 1.38 (d, J=6.4 Hz, 3H), 0.94 (t, J=7.2 Hz, 3H), 0.73 (t, J=7.2 Hz, 3H) ppm. MS: M/e 494(M+1)⁺.

Compound A46b (the later peak): ¹H NMR (400 MHz, CD₃OD) δ 8.52 (s, 1H), 7.89 (d, J=8.0 Hz, 1H), 7.79 (s, 1H), 7.56 (d, J=8.0 Hz, 1H), 5.60 (s, 1H), 3.94 (s, 2H), 3.73 (s, 3H), 3.72-3.66 (m, 1H), 3.53-3.46 (m, 1H), 3.33-3.31 (m, 2H), 3.20-3.13 (m, 1H), 2.75-2.65 (m, 1H), 2.34-2.26 (m, 1H), 2.00-1.85 (m, 1H), 1.70 (d, J=2.4 Hz, 3H), 1.69-1.48 (m, 6H), 1.35 (d, J=6.4 Hz, 3H), 1.04 (t, J=7.2 Hz, 3H), 0.61 (t, J=7.2 Hz, 3H) ppm. MS: M/e 494(M+1)⁺.

Compound A47: 2-(8-((2S,5R)-4-(1-(2-cyclopropylpyrimidin-5-yl)ethyl)-2,5-diethylpiperazin-1-yl)-5-methyl-6-oxo-5,6-dihydroimidazo[1,2-b]pyridazin-2-yl)acetonitrile

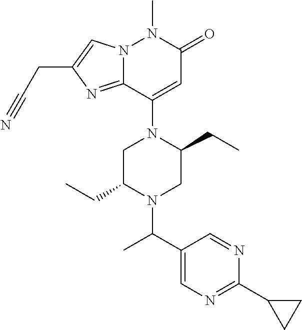 [SMILES: CC[C@H]1CN(C(C)c2cnc(C3CC3)nc2)[C@H](CC)CN1c1cc(=O)n(C)n2cc(CC#N)nc12]

### Step A: 2-cyclopropyl-N-methoxy-N-methylpyrimidine-5-carboxamide

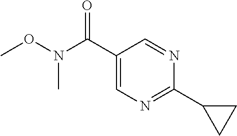

A mixture of 2-cyclopropylpyrimidine-5-carboxylic acid (328 mg, 2 mmol), N,O-dimethylhydroxylamine hydrochloride (234 mg, 2.4 mmol), HATU (917 mg, 2.4 mmol) and DIPEA (516 mg, 4 mmol) in CH₂Cl₂ (15 mL) was stirred overnight. The reaction mixture was washed with H₂O, brine, extracted with CH₂Cl₂ (15 mL×2). The combined organic layers were washed with brine, dried over Na₂SO₄, concentrated and purified by flash column chromatography to give the titled compound (427 mg, 100%) as lightyellow oil. MS: M/e 208 (M+1)⁺.

### Step B: 1-(2-cyclopropylpyrimidin-5-yl)ethan-1-one

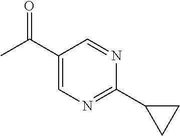

To a stirred solution of 2-cyclopropyl-N-methoxy-N-methylpyrimidine-5-carboxamide (427 mg, 2 mmol) in THF (10 mL) was added dropwise MeMgBr (3.0 M, 1 mL, 3 mmol) at 0° C. After the addition, the reaction mixture was stirred for 30 min. The reaction was quenched with aq.NH₄Cl, extracted with EtOAc (15 mL×3). The combined organic layers were washed with brine, dried over Na₂SO₄, concentrated and purified by flash column chromatography to give the titled compound (204 mg, 63%) as colorless oil. MS: M/e 166 (M+1)⁺.

### Step C: 1-(2-cyclopropylpyrimidin-5-yl)ethan-1-ol

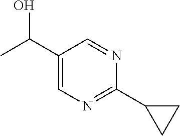

To a stirred solution of 1-(2-cyclopropylpyrimidin-5-yl)ethan-1-one (204 mg, 1.26 mmol) in MeOH (5 mL) was added NaBH₄ (48 mg, 1.26 mmol). After then, the reaction mixture was stirred for 10 min. The reaction mixture was quenched with H₂O, then most MeOH was removed to give the aqueous layer, then extracted with EtOAc (10 mL×2). The combined organic layers were washed with brine, dried over Na₂SO₄, concentrated to give the titled compound (200 mg, 96.7%) as colorless oil. MS: M/e 165 (M+1)⁺.

### Step D: 2-(8-((2S,5R)-4-(1-(2-cyclopropylpyrimidin-5-yl)ethyl)-2,5-diethylpiperazin-1-yl)-5-methyl-6-oxo-5,6-dihydroimidazo[1,2-b]pyridazin-2-yl)acetonitrile

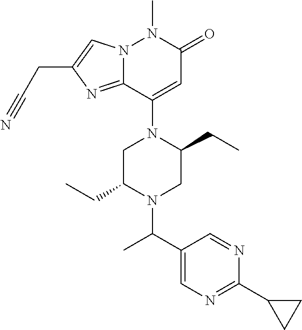 [SMILES: CC[C@H]1CN(C(C)c2cnc(C3CC3)nc2)[C@H](CC)CN1c1cc(=O)n(C)n2cc(CC#N)nc12]

A mixture of 2-(8-((2S,5R)-2,5-diethylpiperazin-1-yl)-5-methyl-6-oxo-5,6-dihydroimidazo[1,2-b]pyridazin-2-yl)acetonitrile (32.8 mg, 0.1 mmol), 1-(2-cyclopropylpyrimidin-5-yl)ethan-1-ol (32.8 mg, 0.2 mmol), (cyanomethyl)trimethylphosphonium iodide (72.9 mg, 0.3 mmol) and DIPEA (64.5 mg, 0.5 mmol) in CH₃CN (8 mL) was stirred overnight at 100° C. in a sealed tube. The reaction mixture was diluted with EtOAc (20 mL), washed with H₂O, brine, dried over Na₂SO₄, concentrated to dryness. The resulting residue was purified and separated into Compound A47a (5 mg) and Compound A47b (6 mg) by Prep-HPLC (Method B).

Compound A47a (the earlier peak): ¹H NMR (400 MHz, CD₃OD) δ 8.60 (s, 2H), 7.79 (s, 1H), 5.62 (s, 1H), 3.93 (s, 2H), 3.86 (q, J=6.4 Hz, 1H), 3.73 (s, 3H), 3.35-3.31 (m, 3H), 2.98-2.89 (m, 2H), 2.43-2.35 (m, 1H), 2.28-2.17 (m, 1H), 2.11-1.98 (m, 1H), 1.87-1.74 (m, 1H), 1.64-1.47 (m, 2H), 1.40 (d, J=6.4 Hz, 3H), 1.13-1.07 (m, 4H), 0.93 (t, J=7.2 Hz, 3H), 0.78 (t, J=7.2 Hz, 3H) ppm. MS: M/e 475(M+1)⁺.

Compound A47b (the later peak): ¹H NMR (400 MHz, CD₃OD) δ 8.62 (s, 2H), 7.79 (s, 1H), 5.60 (s, 1H), 3.94 (s, 2H), 3.74 (s, 3H), 3.69 (q, J=6.4 Hz, 1H), 3.55-3.46 (m, 1H), 3.34-3.31 (m, 2H), 3.19-3.11 (m, 1H), 2.78-2.67 (m, 1H), 2.35-2.17 (m, 2H), 1.96-1.82 (m, 1H), 1.76-1.47 (m, 3H), 1.36 (d, J=6.4 Hz, 3H), 1.13-1.06 (m, 4H), 1.03 (t, J=7.2 Hz, 3H), 0.65 (t, J=7.2 Hz, 3H) ppm. MS: M/e 475(M+1)⁺.

Compound A48: 2-(8-((2S,5R)-2,5-diethyl-4-(1-(5-methoxypyridin-2-yl)ethyl)piperazin-1-yl)-5-methyl-6-oxo-5,6-dihydroimidazo[1,2-b]pyridazin-2-yl)acetonitrile

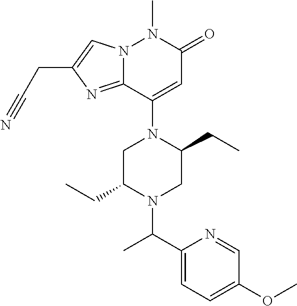 [SMILES: CC[C@H]1CN(C(C)c2ccc(OC)cn2)[C@H](CC)CN1c1cc(=O)n(C)n2cc(CC#N)nc12]

### Step A: 5-methoxypicolinic acid

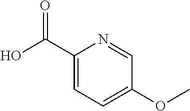

To a stirred solution of methyl 5-methoxypicolinate (500 mg, 3 mmol) in MeOH (8 mL) was added H₂O (2 mL), followed by NaOH (240 mg, 6 mmol). After then, the mixture was stirred for 3 hours. The reaction mixture was concentrated to give the aqueous layer, which was acidified to pH=3-4 with aq.HCl, then extracted with CH₂Cl₂ (10 mL×6). The combined organic layers were washed with brine, dried over Na₂SO₄, concentrated to give the titled compound (395 mg, 86%) as a white solid. MS: M/e 154 (M+1)⁺.

### Step B: N,5-dimethoxy-N-methylpicolinamide

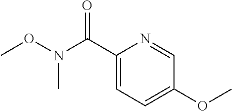

To a stirred solution of 5-methoxypicolinic acid (395 mg, 2.58 mmol), N,O-dimethylhydroxylamine hydrochloride (302 mg, 3.1 mmol), HATU (1.18 g, 3.1 mmol) and DIPEA (665 mg, 5.16 mmol) was stirred overnight. The reaction mixture was washed with H₂O, extracted with CH₂Cl₂ (15 mL×2). The combined organic layers were washed with brine, dried over Na₂SO₄, concentrated and purified by flash column chromatography to give the titled compound (crude, 100%). MS: M/e 197 (M+1)⁺.

### Step C: 1-(5-methoxypyridin-2-yl)ethan-1-one

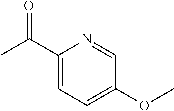

To a stirred solution of N,5-dimethoxy-N-methylpicolinamide (crude, 2.58 mmol) in THF (15 mL) was added dropwise MeMgBr (3.0 M, 1 mL, 3.1 mmol) at 0° C. After then, the mixture was stirred for 30 min. The reaction was quenched with aq.NH4Cl, extracted with EtOAc (15 mL×3). The combined organic layers were washed with brine, dried over Na₂SO₄, concentrated and purified by flash column chromatography to give the titled compound (248 mg, 63.6%). MS: M/e 152 (M+1)⁺.

### Step D: 1-(5-methoxypyridin-2-yl)ethan-1-ol

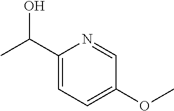

To a stirred solution of 1-(5-methoxypyridin-2-yl)ethan-1-one (248 mg, 1.64 mmol) in MeOH (5 mL) was added NaBH₄ (62.3 mg, 1.64 mmol). After then, the reaction mixture was stirred for 10 min. The reaction mixture was quenched with H₂O, then most MeOH was removed to give the aqueous layer, then extracted with EtOAc (10 mL×3). The combined organic layers were washed with brine, dried over Na₂SO₄, concentrated to give the titled compound (248 mg, 100%). MS: M/e 154 (M+1)⁺.

### Step E: 2-(8-((2S,5R)-2,5-diethyl-4-(1-(5-methoxypyridin-2-yl)ethyl)piperazin-1-yl)-5-methyl-6-oxo-5,6-dihydroimidazo[1,2-b]pyridazin-2-yl)acetonitrile

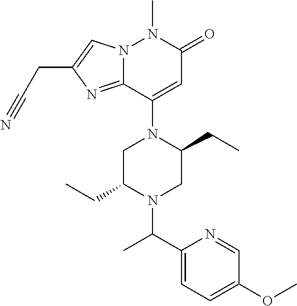 [SMILES: CC[C@H]1CN(C(C)c2ccc(OC)cn2)[C@H](CC)CN1c1cc(=O)n(C)n2cc(CC#N)nc12]

A mixture of 1-(5-methoxypyridin-2-yl)ethan-1-ol (30 mg, 0.2 mmol), 2-(8-((2S,5R)-2,5-diethylpiperazin-1-yl)-5-methyl-6-oxo-5,6-dihydroimidazo[1,2-b]pyridazin-2-yl)acetonitrile (33 mg, 0.1 mmol), (cyanomethyl)trimethylphosphonium iodide (97 mg, 0.4 mmol) and DIPEA (52 mg, 0.4 mmol) in CH₃CN (2 mL) was stirred at 100° C. overnight. The reaction mixture was quenched with water (20 mL), extracted with EtOAc (10 mL×2), dried over Na₂SO₄ and concentrated to dryness. The resulting residue was purified by Prep-HPLC(Method A) to give the titled compound (5 mg, 11%). ¹H NMR (400 MHz, CDCl₃) δ 8.22 (d, J=11.1 Hz, 1H), 7.46-7.31 (m, 2H), 7.23 (s, 1H), 5.76-5.47 (m, 1H), 3.88 (s, 3H), 3.83-3.55 (m, 6H), 3.49-3.20 (m, 1H), 3.12-2.64 (m, 2H), 2.42-2.21 (m, 1H), 1.95 (s, 3H), 1.82-1.42 (m, 4H), 1.33 (d, J=10.5 Hz, 2H), 1.07-0.84 (m, 3H), 0.75-0.56 (m, 3H) ppm. MS: M/e 464 (M+1)⁺.

Compound A49: 2-(8-((2S,5R)-2,5-diethyl-4-(1-(5-isopropoxypyridin-2-yl)ethyl)piperazin-1-yl)-5-methyl-6-oxo-5,6-dihydroimidazo[1,2-b]pyridazin-2-yl)acetonitrile

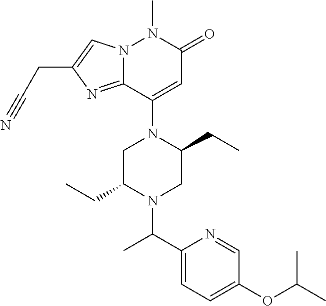 [SMILES: CC[C@H]1CN(C(C)c2ccc(OC(C)C)cn2)[C@H](CC)CN1c1cc(=O)n(C)n2cc(CC#N)nc12]

### Step A: 1-(5-isopropoxypyridin-2-yl)ethan-1-one

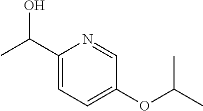

To a solution of 1-(5-hydroxypyridin-2-yl)ethan-1-one (274 mg, 2 mmol) and K₂CO₃ (552 mg, 4 mmol) in CH₃CN (10 mL) was added 2-iodopropane (752 mg, 4 mmol). The mixture was heated at 80° C. for 16 hours under N₂. The reaction mixture was cooled to room temperature, extracted with EA (60 mL×2), washed with brine, dried over Na₂SO₄, filtered, and concentrated. The residue was purified by flash column chromatography (EA/PE=½) to give the titled compound (300 mg, 83%). MS: M/e 180 (M+1)⁺.

### Step B: 1-(5-isopropoxypyridin-2-yl)ethan-1-ol

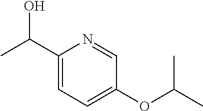

To a solution of the product of step A (300 mg, 1.67 mmol) in MeOH (20 mL) at ice-cooled bath was added NaBH₄ (64 mg, 1.67 mmol) in some portions. The mixture was stirred at room temperature for 2 hours. The reaction was quenched with cold water, extracted with EA (80 mL), washed with brine, dried over Na₂SO₄, filtered, and concentrated. The residue was purified by flash column chromatography (EtOAc/PE=1/1) to give the titiled compound (190 mg, 62%). MS: M/e 182 (M+1)⁺.

### Step C: 2-(8-((2S,5R)-2,5-diethyl-4-(1-(5-isopropoxypyridin-2-yl)ethyl)piperazin-1-yl)-5-methyl-6-oxo-5,6-dihydroimidazo[1,2-b]pyridazin-2-yl)acetonitrile

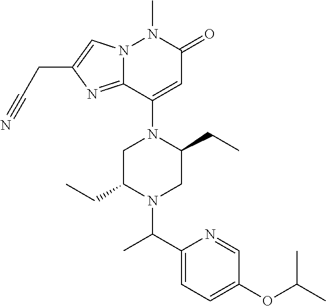 [SMILES: CC[C@H]1CN(C(C)c2ccc(OC(C)C)cn2)[C@H](CC)CN1c1cc(=O)n(C)n2cc(CC#N)nc12]

A mixture of 1-(5-isopropoxypyridin-2-yl)ethan-1-ol (36 mg, 0.2 mmol), 2-(8-((2S,5R)-2,5-diethylpiperazin-1-yl)-5-methyl-6-oxo-5,6-dihydroimidazo[1,2-b]pyridazin-2-yl)acetonitrile (33 mg, 0.1 mmol), (cyanomethyl)trimethylphosphonium iodide (97 mg, 0.4 mmol) and DIPEA (52 mg, 0.4 mmol) in MeCN (2 mL) was stirred at 100° C. overnight. The reaction mixture was quenched with water (20 mL), extracted with EtOAc (10 mL×2), dried over Na₂SO₄ and concentrated to dryness. The resulting residue was purified by Prep-HPLC(Method A) to give the titled compound (9 mg, 18%). ¹H NMR (400 MHz, CDCl₃) δ 8.18 (dd, J=10.9, 2.4 Hz, 1H), 7.45-7.30 (m, 2H), 7.19 (d, J=6.4 Hz, 1H), 5.59 (s, 1H), 4.57 (dd, J=10.1, 5.9 Hz, 1H), 3.91-3.67 (m, 6H), 3.49-3.19 (m, 1H), 3.06 (d, J=9.8 Hz, 0.5H), 2.90 (s, 1H), 2.69 (d, J=10.8 Hz, 0.5H), 2.41-2.25 (m, 1H), 2.05 (dd, J=14.3, 7.3 Hz, 1H), 1.93 (s, 1H), 1.80-1.69 (m, 1H), 1.66-1.49 (m, 4H), 1.42-1.27 (m, 9H), 1.04-0.86 (m, 3H), 0.75-0.57 (m, 3H) ppm. MS: M/e 492 (M+1)⁺.

Compound A50: 2-(8-((2S,5R)-4-(1-(6-cyclopropylpyridin-3-yl)ethyl)-5-ethyl-2-methylpiperazin-1-yl)-7-fluoro-5-methyl-6-oxo-5,6-dihydroimidazo[1,2-b]pyridazin-2-yl)acetonitrile

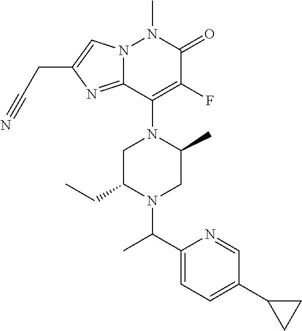 [SMILES: CC[C@@H]1CN(c2c(F)c(=O)n(C)n3cc(CC#N)nc23)[C@@H](C)CN1C(C)c1ccc(C2CC2)cn1]

To a solution of 2-(8-((2S,5R)-4-(1-(6-cyclopropylpyridin-3-yl)ethyl)-5-ethyl-2-methylpiperazin-1-yl)-5-methyl-6-oxo-5,6-dihydroimidazo[1,2-b]pyridazin-2-yl)acetonitrile (100 mg, 0.22 mmol) in CH₃CN (5 mL) was added Selectfluor fluorinating reagent (86.2 mg, 0.55 mmol). The mixture was stirred at room temperature overnight. The reaction was poured into water, extracted with EtOAc (20 mL), washed with brine, dried over Na₂SO₄ and concentrated to dryness. The resulting residue was purified and separated into Compound A50a (6 mg) and Compound A50b (7 mg) by Prep-HPLC (Method A).

Compound A50a (the earlier peak): ¹H NMR (400 MHz, CD₃OD) δ 8.31 (s, 1H), 7.77 (s, 1H), 7.72 (d, J=8.0 Hz, 1H), 7.21 (d, J=8.0 Hz, 1H), 4.80-4.68 (m, 1H), 3.92 (s, 2H), 3.90-3.81 (m, 2H), 3.78 (s, 3H), 3.71-3.64 (m, 1H), 3.11-3.04 (m, 1H), 2.77-2.67 (m, 1H), 2.39-2.30 (m, 1H), 2.14-2.05 (m, 1H), 1.75-1.51 (m, 2H), 1.43-1.33 (m, 6H), 1.07-0.91 (m, 4H), 0.67 (t, J=7.2 Hz, 3H) ppm. MS: M/e 478 (M+1)⁺.

Compound A50b (the later peak): ¹H NMR (400 MHz, CD₃OD) δ 8.35 (s, 1H), 7.78 (s, 1H), 7.74 (d, J=8.0 Hz, 1H), 7.17 (d, J=8.0 Hz, 1H), 4.62-4.49 (m, 1H), 4.17-4.07 (m, 1H), 3.93 (s, 2H), 3.90-3.84 (m, 1H), 3.78 (s, 3H), 3.76-3.69 (m, 1H), 3.12-3.05 (m, 1H), 2.87-2.80 (m, 1H), 2.20-2.13 (m, 1H), 2.12-2.03 (m, 1H), 1.64-1.37 (m, 2H), 1.32 (d, J=6.4 Hz, 3H), 1.23 (d, J=6.4 Hz, 3H), 1.05-0.89 (m, 7H) ppm. MS: M/e 478 (M+1)⁺.

Compound A51: 2-(8-((2S,5R)-2,5-diethyl-4-(1-(4-isopropoxyphenyl)ethyl)piperazin-1-yl)-5-methyl-6-oxo-5,6-dihydroimidazo[1,2-b]pyridazin-2-yl)acetonitrile

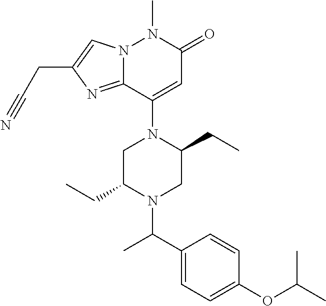 [SMILES: CC[C@H]1CN(C(C)c2ccc(OC(C)C)cc2)[C@H](CC)CN1c1cc(=O)n(C)n2cc(CC#N)nc12]

### Step A: 1-(4-isopropoxyphenyl)ethan-1-one

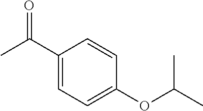

A mixture of 1-(4-hydroxyphenyl)ethan-1-one (1.38 g, 10 mmol), 2-iodopropane (2.55 g, 15 mmol) and potassium carbonate (2.76 g, 20 mmol) in DMF (20 mL) was stirred at 70° C. for 16 hours. The mixture was treated with water (100 ml), extracted with EtOAc (30 ml*3), washed with brine (20 mL), dried and concentrated to dryness. The resulting residue was purified by flash column chromatography (0-50% EtOAc in PE in 30 minutes) to give the titled compound (1.7 g, 95%). MS: M/e 179 (M+1)⁺.

### Step B: 1-(4-isopropoxyphenyl)ethan-1-ol

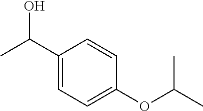

To a solution of 1-(4-isopropoxyphenyl)ethan-1-one (1.7 g, 10 mmol) in MeOH (15 mL) was added NaBH₄ (304 mg, 8 mmol) at room temperature and the mixture was stirred at room temperature for 30 minutes. The resulting mixture was treated with water (50 ml), extracted with EtOAc (20 mL×3). The combined organic layers were dried over Na₂SO₄ and concentrated to dryness. The resulting residue was purified by flash column chromatography (0-50% EtOAc in PE in 30 minutes) to give the titled compound (1.5 g, 88%). MS: M/e 181 (M+1)⁺.

### Step C: 2-(8-((2S,5R)-2,5-diethyl-4-(1-(4-isopropoxyphenyl)ethyl)piperazin-1-yl)-5-methyl-6-oxo-5,6-dihydroimidazo[1,2-b]pyridazin-2-yl)acetonitrile

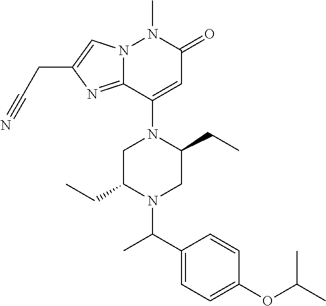 [SMILES: CC[C@H]1CN(C(C)c2ccc(OC(C)C)cc2)[C@H](CC)CN1c1cc(=O)n(C)n2cc(CC#N)nc12]

A mixture of 1-(4-isopropoxyphenyl)ethan-1-ol (30 mg, 0.2 mmol), 2-(8-((2S,5R)-2,5-diethylpiperazin-1-yl)-5-methyl-6-oxo-5,6-dihydroimidazo[1,2-b]pyridazin-2-yl)acetonitrile (33 mg, 0.1 mmol), (cyanomethyl)trimethylphosphonium iodide (97 mg, 0.4 mmol) and DIPEA (51 mg, 0.4 mmol) in MeCN (2 mL) was stirred at 100° C. overnight. The reaction mixture was diluted with EtOAc (20 mL), washed with water (10 mL×2), dried over Na₂SO₄ and concentrated to dryness. The resulting residue was purified by Prep-HPLC(Method A) to give the titled compound (8 mg, 16%). ¹H NMR (400 MHz, CDCl₃) δ 7.34 (s, 1H), 7.25-7.16 (m, 2H), 6.87-6.79 (m, 2H), 5.58 (s, 1H), 4.53 (dq, J=12.2, 6.1 Hz, 1H), 3.77 (d, J=2.9 Hz, 2H), 3.71 (s, 3H), 3.66-3.45 (m, 1H), 3.41 (d, J=12.9 Hz, 0.5H), 3.23-3.03 (m, 1H), 2.96-2.77 (m, 1H), 2.54 (d, J=12.5 Hz, 0.5H), 2.38 (d, J=12.1 Hz, 1H), 2.12-1.88 (m, 1H), 1.74 (d, J=6.7 Hz, 2H), 1.49 (d, J=9.3 Hz, 2H), 1.37-1.24 (m, 9H), 1.02-0.87 (m, 3H), 0.71-0.55 (m, 3H) ppm. MS: M/e 491 (M+1)⁺.

Compound A52: 2-(8-((2S,5R)-4-(1-(5-cyclopropylpyridin-2-yl)ethyl)-2,5-diethylpiperazin-1-yl)-5-methyl-6-oxo-5,6-dihydroimidazo[1,2-b]pyridazin-2-yl)acetonitrile

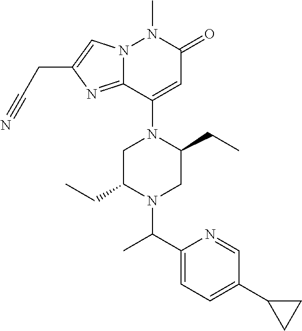 [SMILES: CC[C@H]1CN(C(C)c2ccc(C3CC3)cn2)[C@H](CC)CN1c1cc(=O)n(C)n2cc(CC#N)nc12]

### Step A: 1-(5-cyclopropylpyridin-2-yl)ethan-1-one

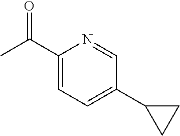

A mixture of 1-(5-bromopyridin-2-yl)ethan-1-one (1 g, 5 mmol), cyclopropylboronic acid (645 mg, 7.5 mmol), Dichlorobis(tricyclohexylphosphine)palladium(II) (369 mg, 0.5 mmol) and potassium phosphate (1.6 g, 7.5 mmol) in toluene (10 mL) and water (1 ml) was stirred at 100° C. under N₂ overnight. The mixture was treated with water (100 ml), extracted with EtOAc (20 ml*3), washed with brine (50 mL), dried and concentrated to dryness. The resulting residue was purified by flash column chromatography (0-50% EtOAc in PE in 30 minutes) to give the titled compound (300 mg, 37%). MS: M/e 162 (M+1)⁺.

### Step B: 1-(5-cyclopropylpyridin-2-yl)ethan-1-ol

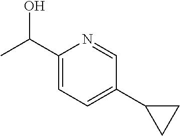

To a solution of 1-(5-cyclopropylpyridin-2-yl)ethan-1-one (300 mg, 1.86 mmol) in MeOH (5 mL) was added NaBH₄ (56 mg, 1.49 mmol) at room temperature and the mixture was stirred at room temperature for 30 minutes. The resulting mixture was treated with water (20 ml), extracted with EtOAc (10 mL×2). The combined organic layers were dried over Na₂SO₄ and concentrated to dryness. The resulting residue was purified by flash column chromatography (0-50% EtOAc in PE in 30 minutes) to give the titled compound (160 mg, 53%). MS: M/e 164 (M+1)⁺.

### Step C: 2-(8-((2S,5R)-4-(1-(5-cyclopropylpyridin-2-yl)ethyl)-2,5-diethylpiperazin-1-yl)-5-methyl-6-oxo-5,6-dihydroimidazo[1,2-b]pyridazin-2-yl)acetonitrile

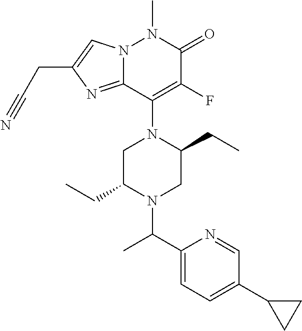 [SMILES: CC[C@H]1CN(C(C)c2ccc(C3CC3)cn2)[C@H](CC)CN1c1c(F)c(=O)n(C)n2cc(CC#N)nc12]

A mixture of 1-(5-cyclopropylpyridin-2-yl)ethan-1-ol (32 mg, 0.1 mmol), 2-(8-((2S,5R)-2,5-diethylpiperazin-1-yl)-5-methyl-6-oxo-5,6-dihydroimidazo [1,2-b]pyridazin-2-yl) acetonitrile (33 mg, 0.1 mmol), (cyanomethyl)trimethylphosphonium iodide (97 mg, 0.4 mmol) and DIPEA (52 mg, 0.4 mmol) in MeCN (2 mL) was stirred at 100° C. overnight. The reaction mixture was diluted with EtOAc (20 mL), washed with water (10 mL×2), dried over Na₂SO₄ and concentrated to dryness. The resulting residue was purified by Prep-HPLC(Method A) to give the titled compound (8 mg, 16%). ¹H NMR (400 MHz, CD₃OD) δ 8.26 (d, J=7.9 Hz, 1H), 7.79 (s, 1H), 7.56-7.42 (m, 2H), 5.60 (s, 1H), 4.03-3.79 (m, 3H), 3.79-3.48 (m, 4H), 3.31 (s, 2H), 3.19-2.97 (m, 1H), 2.95-2.68 (m, 1H), 2.39-2.19 (m, 1H), 2.15-1.89 (m, 2H), 1.87-1.46 (m, 3H), 1.40-1.27 (m, 3H), 1.10-0.90 (m, 5H), 0.79-0.57 (m, 5H) ppm. MS: M/e 474 (M+1)⁺.

Compound A53: 2-(8-((2S,5R)-4-(1-(benzo[d]thiazol-6-yl)ethyl)-2,5-diethylpiperazin-1-yl)-5-methyl-6-oxo-5,6-dihydroimidazo[1,2-b]pyridazin-2-yl)acetonitrile

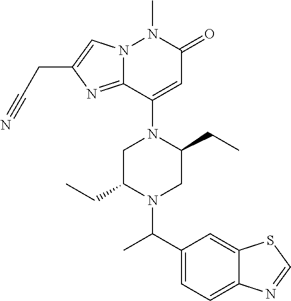 [SMILES: CC[C@H]1CN(C(C)c2ccc3ncsc3c2)[C@H](CC)CN1c1cc(=O)n(C)n2cc(CC#N)nc12]

### Step A: 1-(benzo[d]thiazol-6-yl)ethan-1-ol

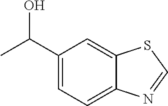

To a solution of 1-(benzo[d]thiazol-6-yl)ethan-1-one (500 mg, 2.8 mmol) in MeOH (3 mL) was added NaBH₄ (75 mg, 1.9 mmol) at room temperature and the resulting mixture was stirred at room temperature for 5 min. The reaction mixture was diluted with DCM and washed with water, dried over Na₂SO₄ and concentrated and purified by flash column chromatography to give the titled compound (350 mg, 69%). MS: M/e 180 (M+1)⁺.

### Step 2: 2-(8-((2S,5R)-4-(1-(benzo[d]thiazol-6-yl)ethyl)-2,5-diethylpiperazin-1-yl)-5-methyl-6-oxo-5,6-dihydroimidazo[1,2-b]pyridazin-2-yl)acetonitrile

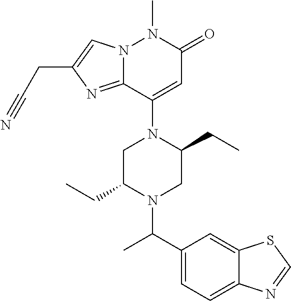 [SMILES: CC[C@H]1CN(C(C)c2ccc3ncsc3c2)[C@H](CC)CN1c1cc(=O)n(C)n2cc(CC#N)nc12]

A mixture of 2-(8-((2S,5R)-2,5-diethylpiperazin-1-yl)-5-methyl-6-oxo-5,6-dihydroimidazo[1,2-b]pyridazin-2-yl)acetonitrile (328 mg, 1 mmol), 1-(benzo[d]thiazol-6-yl)ethan-1-ol (268.5 mg, 1.5 mmol), (cyanomethyl)trimethylphosphonium iodide (729 mg, 3 mmol) and DIPEA (645 mg, 5 mmol) in CH₃CN (8 mL) was stirred overnight at 100° C. in a sealed tube. The reaction mixture was diluted with EtOAc (20 mL), washed with H₂O, brine, dried over Na₂SO₄, concentrated to dryness. The resulting residue was purified by Pre-TLC (CH₂Cl₂/MeOH=10:1) to give the titled Compound A53 (243 mg), which was further separated into Compound A53a (70 mg) and Compound A53b (70 mg) by Prep-HPLC (Method B).

Compound A53: ¹H NMR (400 MHz, CD₃OD) δ 9.25-9.18 (m, 1H), 8.14-7.99 (m, 2H), 7.81-7.75 (m, 1H), 7.69-7.60 (m, 1H), 5.63-5.57 (m, 1H), 3.97-3.89 (m, 2H), 3.85-3.77 (m, 0.5H), 3.73 (s, 3H), 3.57-3.50 (m, 0.5H), 3.24-2.91 (m, 3H), 2.74-2.06 (m, 2H), 2.06-1.49 (m, 4H), 1.48-1.34 (m, 3H), 1.12-0.90 (m, 4H), 0.74-0.51 (m, 3H) ppm. MS: M/e 490 (M+1)⁺.

Compound A53a (the earlier peak): ¹H NMR (400 MHz, CD₃OD) δ 9.19 (s, 1H), 8.06-8.01 (m, 2H), 7.76 (s, 1H), 7.60 (dd, J=8.4, 1.6 Hz, 1H), 5.58 (s, 1H), 3.95-3.88 (m, 3H), 3.71 (s, 3H), 3.29-3.25 (m, 3H), 3.06-3.01 (m, 1H), 2.93-2.86 (m, 1H), 2.45-2.37 (m, 1H), 2.18-2.07 (m, 1H), 1.89-1.79 (m, 1H), 1.58-1.47 (m, 2H), 1.39 (d, J=6.4 Hz, 3H), 0.94 (t, J=7.2 Hz, 3H), 0.65 (t, J=7.2 Hz, 3H) ppm. MS: M/e 490 (M+1)⁺.

Compound A53b (the later peak): ¹H NMR (400 MHz, CD₃OD) δ 9.22 (s, 1H), 8.12 (d, J=1.2 Hz, 1H), 8.04 (d, J=8.4 Hz, 1H), 7.80 (s, 1H), 7.66 (dd, J=8.4, 1.2 Hz, 1H), 5.60 (s, 1H), 3.95 (s, 2H), 3.84-3.77 (m, 1H), 3.75 (s, 3H), 3.57-3.50 (m, 1H), 3.36-3.32 (m, 2H), 3.25-3.16 (m, 1H), 2.71-2.66 (m, 1H), 2.42-2.33 (m, 1H), 2.07-1.96 (m, 1H), 1.76-1.54 (m, 3H), 1.40 (d, J=6.4 Hz, 3H), 1.07 (t, J=7.2 Hz, 3H), 0.58 (t, J=7.2 Hz, 3H) ppm. MS: M/e 490 (M+1)⁺.

Compound A54: 2-(8-((2S,5R)-4-(1-(benzo[d]thiazol-6-yl)ethyl)-5-ethyl-2-methylpiperazin-1-yl)-5-methyl-6-oxo-5,6-dihydroimidazo[1,2-b]pyridazin-2-yl)acetonitrile

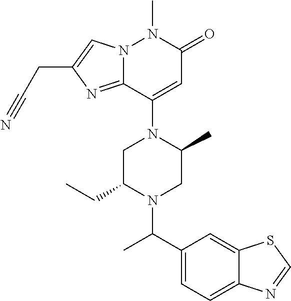 [SMILES: CC[C@@H]1CN(c2cc(=O)n(C)n3cc(CC#N)nc23)[C@@H](C)CN1C(C)c1ccc2ncsc2c1]

A mixture of the product of 2-(8-((2S,5R)-5-ethyl-2-methylpiperazin-1-yl)-5-methyl-6-oxo-5,6-dihydroimidazo[1,2-b]pyridazin-2-yl)acetonitrile (31.4 mg, 0.1 mmol), 1-(benzo[d]thiazol-6-yl)ethan-1-ol (35.8 mg, 0.2 mmol), (cyanomethyl)trimethylphosphonium iodide (72.9 mg, 0.3 mmol) and DIPEA (64.5 mg, 0.5 mmol) in CH₃CN (4 mL) was stirred at 100° C. overnight in a sealed tube. The reaction mixture was diluted with EtOAc (10 mL), washed with brine, dried over Na₂SO₄, concentrated to dryness. The resulting residue was purified by Pre-TLC (CH₂Cl₂:MeOH=10:1) to give the titled compound (12 mg). ¹H NMR (400 MHz, CD₃OD) δ 9.23-9.19 (m, 1H), 8.13-7.99 (m, 2H), 7.79-7.77 (m, 1H), 7.68-7.61 (m, 1H), 5.64-5.61 (m, 1H), 4.00-3.90 (m, 3H), 3.85-3.76 (m, 0.5H), 3.73 (s, 3H), 3.58-3.53 (m, 0.5H), 3.35-3.32 (m, 1H), 3.23-3.19 (m, 0.5H), 3.06-2.76 (m, 1.5H), 2.53-2.21 (m, 1H), 1.76-1.53 (m, 3H), 1.47-1.38 (m, 5H), 1.22 (d, J=6.4 Hz, 1.5H), 1.08 (t, J=7.2 Hz, 1.5H), 0.74 (t, J=7.2 Hz, 1.5H) ppm. MS: M/e 476 (M+1)⁺.

Compound A55: 2-(8-((2S,5R)-5-ethyl-2-methyl-4-(1-(2-methylbenzo[d]thiazol-6-yl)ethyl)piperazin-1-yl)-5-methyl-6-oxo-5,6-dihydroimidazo[1,2-b]pyridazin-2-yl)acetonitrile

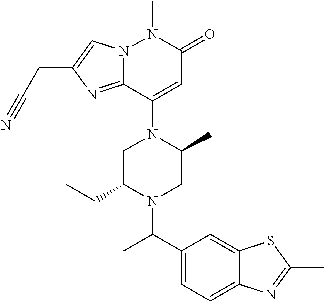 [SMILES: CC[C@@H]1CN(c2cc(=O)n(C)n3cc(CC#N)nc23)[C@@H](C)CN1C(C)c1ccc2nc(C)sc2c1]

### Step A: 1-(2-methylbenzo[d]thiazol-6-yl)ethan-1-one

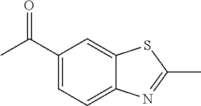

A solution of 6-bromo-2-methylbenzo[d]thiazole (1 g, 4.39 mmol), ethyl tributylstannanecarboxylate (1.98 g, 5.48 mmol) and Pd(PPh₃)₂Cl₂ (150 mg, 0.21 mmol) in toluene (10 ml) was stirred at 100° C. overnight. The solution was cooled to RT. HCl/1,4-dioxane (4M, 2 ml) was added and stirred at RT for 10 min. The solution was diluted with EtOAc (20 ml), washed with aq. Na₂CO₃ (10 ml×2), dried over Na₂SO₄ and concentrated to dryness. The resulting residue was purified by flash column chromatography (0-30% EA in PE) to give the titled compound (600 mg, 72%). MS: M/e 192 (M+1)⁺.

### Step B: 1-(2-methylbenzo[d]thiazol-6-yl)ethan-1-ol

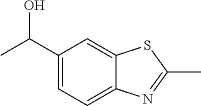

A solution of 1-(2-methylbenzo[d]thiazol-6-yl)ethan-1-one (600 mg, 3.14 mmol) and NaBH₄ (119 mg, 3.13 mmol) in MeOH (6 ml) was stirred at RT for 10 min. The solution was diluted with EtOAc (15 ml), washed with brine (10 ml×2), dried over Na₂SO₄ and concentrated to dryness to give the titled compound (600 mg, 99%), which was used directly for the next step without further purification. MS: M/e 194 (M+1)⁺.

### Step C: 2-(8-((2S,5R)-5-ethyl-2-methyl-4-(1-(2-methylbenzo[d]thiazol-6-yl)ethyl)piperazin-1-yl)-5-methyl-6-oxo-5,6-dihydroimidazo[1,2-b]pyridazin-2-yl)acetonitrile

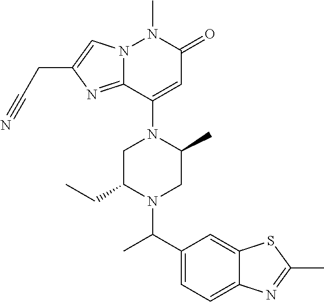 [SMILES: CC[C@@H]1CN(c2cc(=O)n(C)n3cc(CC#N)nc23)[C@@H](C)CN1C(C)c1ccc2nc(C)sc2c1]

A mixture of 2-(8-((2S,5R)-5-ethyl-2-methylpiperazin-1-yl)-5-methyl-6-oxo-5,6-dihydroimidazo[1,2-b]pyridazin-2-yl)acetonitrile (50 mg, 0.16 mmol), 1-(2-methylbenzo[d]thiazol-6-yl)ethan-1-ol (61 mg, 0.318 mmol), (cyanomethyl)trimethylphosphonium iodide (116 mg, 0.48 mmol) and DIPEA (103 mg, 0.8 mmol) in CH₃CN (8 mL) was stirred overnight at 100° C. in a sealed tube. The reaction mixture was diluted with EtOAc (20 mL), washed with H₂O, brine, dried over Na₂SO₄, concentrated to dryness. The resulting residue was purified by flash column chromatography to give the titled compound (23 mg). ¹H NMR (400 MHz, CD₃OD) δ 7.98-7.91 (m, 1H), 7.89-7.76 (m, 2H), 7.61-7.51 (m, 1H), 5.66-5.58 (m, 1H), 5.32-5.25 (m, 1H), 4.52-4.49 (m, 1H), 3.95-3.90 (m, 2H), 3.90-3.75 (m, 1H), 3.73 (s, 3H), 3.59-3.50 (m, 0.5H), 3.23-3.14 (m, 0.5H), 3.04-2.86 (m, 1H), 2.85-2.80 (m, 3H), 2.79-2.71 (m, 0.5H), 2.49-2.20 (m, 1H), 1.75-1.47 (m, 2.5H), 1.43-1.18 (m, 6H), 1.11-0.69 (m, 3H) ppm. MS: M/e 490(M+1)⁺.

Compound A56: 2-(8-((2S,5R)-2,5-diethyl-4-(1-(2-methylbenzo[d]thiazol-6-yl)ethyl)piperazin-1-yl)-5-methyl-6-oxo-5,6-dihydroimidazo[1,2-b]pyridazin-2-yl)acetonitrile

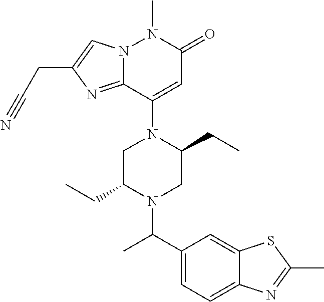 [SMILES: CC[C@H]1CN(C(C)c2ccc3nc(C)sc3c2)[C@H](CC)CN1c1cc(=O)n(C)n2cc(CC#N)nc12]

A mixture of 2-(8-((2S,5R)-2,5-diethylpiperazin-1-yl)-5-methyl-6-oxo-5,6-dihydroimidazo[1,2-b]pyridazin-2-yl)acetonitrile (100 mg, 0.3 mmol), 1-(2-methylbenzo[d]thiazol-6-yl)ethan-1-ol (116 mg, 0.6 mmol), (cyanomethyl)trimethylphosphonium iodide (219 mg, 0.9 mmol) and DIPEA (192 mg, 1.5 mmol) in CH₃CN (8 mL) was stirred overnight at 100° C. in a sealed tube. The reaction mixture was diluted with EtOAc (20 mL), washed with H₂O, brine, dried over Na₂SO₄, concentrated to dryness. The resulting residue was purified and separated into Compound A56a (20 mg) and Compound A56b (19 mg) by Prep-HPLC (Method B).

Compound A56a (the earlier peak): ¹H NMR (400 MHz, CD₃OD) δ 7.91 (s, 1H), 7.85 (d, J=8.4 Hz, 1H), 7.77 (s, 1H), 7.53 (d, J=8.4 Hz, 1H), 5.60 (s, 1H), 3.92 (s, 2H), 3.91-3.85 (m, 1H), 3.73 (s, 3H), 3.33-3.30 (m, 2H), 3.29-3.25 (m, 1H), 3.06-2.87 (m, 2H), 2.82 (s, 3H), 2.46-2.38 (m, 1H), 2.18-2.07 (m, 1H), 1.89-1.79 (m, 1H), 1.62-1.47 (m, 2H), 1.39 (d, J=6.4 Hz, 3H), 0.95 (t, J=7.2 Hz, 3H), 0.67 (t, J=7.2 Hz, 3H) ppm. MS: M/e 504(M+1)⁺.

Compound A56b (the later peak): ¹H NMR (400 MHz, CD₃OD) δ 7.94 (s, 1H), 7.82 (d, J=8.4 Hz, 1H), 7.77 (s, 1H), 7.54 (d, J=8.4 Hz, 1H), 5.57 (s, 1H), 3.92 (s, 2H), 3.76-3.68 (m, 4H), 3.53-3.46 (m, 1H), 3.32-3.30 (m, 2H), 3.21-3.13 (m, 1H), 2.81 (s, 3H), 2.69-2.60 (m, 1H), 2.38-2.32 (m, 1H), 2.05-1.93 (m, 1H), 1.72-1.51 (m, 3H), 1.35 (d, J=6.4 Hz, 3H), 1.04 (t, J=7.2 Hz, 3H), 0.56 (t, J=7.2 Hz, 3H) ppm. MS: M/e 504(M+1)⁺.

Compound A57: 2-(8-((2S,5R)-2,5-diethyl-4-(1-(thiazolo[5,4-b]pyridin-5-yl)ethyl)piperazin-1-yl)-5-methyl-6-oxo-5,6-dihydroimidazo[1,2-b]pyridazin-2-yl)acetonitrile

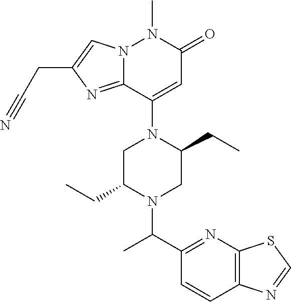 [SMILES: CC[C@H]1CN(C(C)c2ccc3ncsc3n2)[C@H](CC)CN1c1cc(=O)n(C)n2cc(CC#N)nc12]

### Step A: 1-(thiazolo[5,4-b]pyridin-5-yl)ethan-1-one

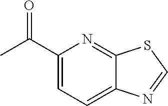

A mixture of 5-bromothiazolo[5,4-b]pyridine (1.3 g, 5.9 mmol), tributyl(1-ethoxyvinyl)stannane (3.2 g, 0.9 mmol) and Pd(PPh₃)₂Cl₂ (40 mg, 0.06 mmol) in DMF (3 mL) was stirred at 100° C. under N₂ for 16 hours. The solution was diluted with ethyl acetate and washed with water. The organic layer was dried and added with HCl (3 mL, 3 M in ethyl acetate) in drops and the mixture was stirred at room temperature for 2 hours. The reaction was concentrated and adjusted to pH=8-9 with saturated NaHCO₃ solution, extracted with ethyl acetate. The combined organic layers were washed with brine, dried over Na₂SO₄ and the resulting residue was purified by flash column chromatography to give the titled compound (300 mg, 28%). MS: M/e 179(M+1)⁺

### Step B: 1-(thiazolo[5,4-b]pyridin-5-yl)ethan-1-ol

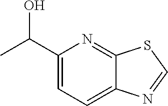

To a solution of 1-(thiazolo[5,4-b]pyridin-5-yl)ethan-1-one (300 mg, 1.65 mmol) in MeOH (15 mL) was added NaBH₄ (60 mg, 1.65 mmol) at room temperature and the resulting mixture was stirred at room temperature for 5 min. The reaction mixture was diluted with DCM and washed with water, dried over Na₂SO₄ and concentrated to give the titled compound (200 mg). MS: M/e 181 (M+1)⁺.

### Step C: 2-(8-((2S,5R)-2,5-diethyl-4-(1-(thiazolo[5,4-b]pyridin-5-yl)ethyl)piperazin-1-yl)-5-methyl-6-oxo-5,6-dihydroimidazo[1,2-b]pyridazin-2-yl)acetonitrile

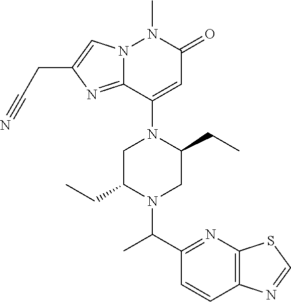 [SMILES: CC[C@H]1CN(C(C)c2ccc3ncsc3n2)[C@H](CC)CN1c1cc(=O)n(C)n2cc(CC#N)nc12]

To a solution of 2-(8-((2S,5R)-2,5-diethylpiperazin-1-yl)-5-methyl-6-oxo-5,6-dihydroimidazo[1,2-b]pyridazin-2-yl)acetonitrile (100 mg, 0.30 mmol) in CH₃CN (5 mL) and was added 1-(thiazolo[5,4-b]pyridin-5-yl)ethan-1-ol (164 mg, 0.91 mmol), (cyanomethyl)trimethylphosphonium iodide (222 mg, 0.91 mmol) and DIPEA (0.39 g, 3.0 mmol). The resulting mixture was stirred at 105° C. overnight. The reaction solvent was removed under reduce pressure. The resulting residue was purified by flash column chromatography to give the titled Compound A57(crude), which was further separated into the Compound A57a (36 mg) and Compound A57b (29 mg) by Prep-chiral HPLC. The chiral separation conditions are shown below.

| | |
|---|---|
| Column | CHIRALPAK IE |
| Column Size | 21.2 mm × 250 mm, 5 um |
| Mobile Phase A | Hexane 30% |
| Mobile Phase B | ETOH(0.5% 2M NH₃MEOH) 70% |
| Flow Rate | 18 mL/min |
| Wave Length | UV 200 nm and 300 nm |
| Temperature | RT |
| Prep-HPLC Equipment | Prep-HPLC-Gilson |

Compound A57a (the earlier peak): ¹H NMR (400 MHz, CD₃OD) δ 9.34 (s, 1H), 8.42 (d, J=8.5 Hz, 1H), 7.79 (d, J=8.7 Hz, 2H), 5.61 (s, 1H), 4.20-4.06 (m, 1H), 3.92 (s, 2H), 3.73 (s, 3H), 3.35-3.30 (m, 3H), 3.07-2.94 (m, 2H), 2.40 (d, J=8.6 Hz, 1H), 2.22-1.75 (m, 2H), 1.59 (dd, J=15.8, 8.1 Hz, 2H), 1.45 (d, J=6.5 Hz, 3H), 0.94 (t, J=7.3 Hz, 3H), 0.71 (t, J=7.2 Hz, 3H) ppm. MS: M/e 491 (M+1)⁺.

Compound A57b (the later peak):¹H NMR (400 MHz, CD₃OD) δ 9.34 (s, 1H), 8.41 (d, J=8.5 Hz, 1H), 7.85 (d, J=8.5 Hz, 1H), 7.79 (s, 1H), 5.60 (s, 1H), 3.93 (s, 3H), 3.73 (s, 3H), 3.55 (d, J=12.5 Hz, 1H), 3.35-3.30 (m, 2H), 3.19 (d, J=10.2 Hz, 1H), 2.80 (dd, J=12.2, 3.6 Hz, 1H), 2.27 (d, J=12.6 Hz, 1H), 1.98 (dt, J=15.0, 8.5 Hz, 1H), 1.71 (dt, J=24.6, 8.8 Hz, 2H), 1.56 (dd, J=13.4, 7.5 Hz, 1H), 1.42 (d, J=6.6 Hz, 3H), 1.05 (t, J=7.3 Hz, 3H), 0.62 (t, J=7.4 Hz, 3H) ppm. MS: M/e 491 (M+1)⁺.

Compound A58: 2-(8-((2S,5R)-2,5-diethyl-4-(1-(2-methylthiazolo[5,4-b]pyridin-5-yl)ethyl)piperazin-1-yl)-5-methyl-6-oxo-5,6-dihydroimidazo[1,2-b]pyridazin-2-yl)acetonitrile

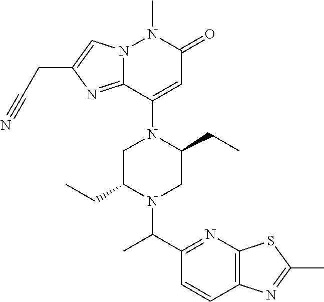 [SMILES: CC[C@H]1CN(C(C)c2ccc3nc(C)sc3n2)[C@H](CC)CN1c1cc(=O)n(C)n2cc(CC#N)nc12]

### Step A: 1-(2-methylthiazolo[5,4-b]pyridin-5-yl)ethan-1-one

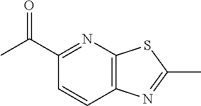

A mixture of 5-chloro-2-methylthiazolo[5,4-b]pyridine (250 mg, 1.35 mmol), tributyl(1-ethoxyvinyl)stannane (738 mg, 2.04 mmol) and Pd(PPh₃)₂Cl₂ (95 mg, 0.14 mmol) in DMF (3 mL) was stirred at 100° C. under N₂ for 16 hours. The solution was diluted with ethyl acetate and washed with water. The organic layer was purified by flash column chromatography. The product was diluted with ethyl acetate and added with HCl (3 mL, 3 M in dioxane) in drops and the mixture was stirred at room temperature for 2 hours. The reaction was concentrated and adjusted to pH=8-9 with saturated NaHCO₃ solution, extracted with ethyl acetate. The combined organic layers were washed with brine, dried over Na₂SO₄. The resulting residue was purified by flash column chromatography to give the titled compound (200 mg, 77%). MS: M/e 193(M+1)⁺

### Step B: 1-(2-methylthiazolo[5,4-b]pyridin-5-yl)ethan-1-ol

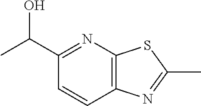

To a solution of 1-(2-methylthiazolo[5,4-b]pyridin-5-yl)ethan-1-one (200 mg, 1.04 mmol) in MeOH (3 mL) was added NaBH₄ (39 mg, 1.04 mmol) at room temperature and the resulting mixture was stirred at room temperature for 5 min. The reaction mixture was diluted with DCM and washed with water, dried over Na₂SO₄. The resulting residue was purified by flash column chromatography to give the titled compound (180 mg, 90%). MS: M/e 195 (M+1)⁺.

### Step C: 2-(8-((2S,5R)-2,5-diethyl-4-(1-(2-methylthiazolo[5,4-b]pyridin-5-yl)ethyl)piperazin-1-yl)-5-methyl-6-oxo-5,6-dihydroimidazo[1,2-b]pyridazin-2-yl)acetonitrile

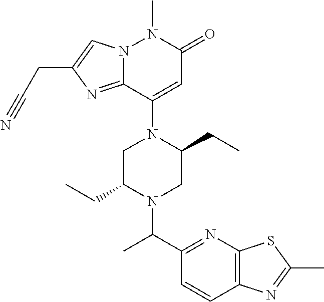 [SMILES: CC[C@H]1CN(C(C)c2ccc3nc(C)sc3n2)[C@H](CC)CN1c1cc(=O)n(C)n2cc(CC#N)nc12]

To a solution of 2-(8-((2S,5R)-2,5-diethylpiperazin-1-yl)-5-methyl-6-oxo-5,6-dihydroimidazo[1,2-b]pyridazin-2-yl)acetonitrile (50 mg, 0.15 mmol), 1-(2-methylthiazolo[5,4-b]pyridin-5-yl)ethan-1-ol (60 mg, 0.30 mmol) and (cyanomethyl)trimethylphosphonium iodide (109 mg, 0.45 mmol) in CH₃CN (1 mL) was added DIPEA (116 mg, 0.90 mmol). The mixture was stirred at 100° C. in a sealed tube for 16 hours. The mixture was cooled and diluted with EtOAc (10 mL), washed with brine (5 mL×3), dried over Na₂SO₄, filtered and concentrated under reduced pressure. The resulting residue was purified by flash column chromatography to give the titled Compound A58, which was further separated into Compound A58a (17 mg) and Compound A58b (17 mg) by Prep-HPLC(Method A).

Compound A58a (the earlier peak):¹H NMR (400 MHz, CD₃OD) δ 8.22 (d, J=8.4 Hz, 1H), 7.79 (s, 1H), 7.76 (d, J=8.4 Hz, 1H), 5.60 (s, 1H), 3.93 (s, 2H), 3.89 (dd, J=12.8, 6.4 Hz, 1H), 3.73 (s, 3H), 3.54 (d, J=11.6 Hz, 1H), 3.35-3.30 (m, 2H), 3.18 (d, J=10.4 Hz, 1H), 2.86 (s, 3H), 2.82-2.74 (m, 1H), 2.28 (d, J=12.4 Hz, 1H), 2.06-1.89 (m, 1H), 1.78-1.49 (m, 3H), 1.40 (d, J=6.8 Hz, 3H), 1.04 (t, J=7.2 Hz, 3H), 0.62 (t, J=7.2 Hz, 3H) ppm. MS: M/e 505 (M+1)⁺.

Compound A58b (the later peak): ¹H NMR (400 MHz, CD₃OD) δ 8.23 (d, J=8.4 Hz, 1H), 7.78 (s, 1H), 7.71 (d, J=8.4 Hz, 1H), 5.61 (s, 1H), 4.14-4.01 (m, 1H), 3.92 (s, 2H), 3.73 (s, 3H), 3.39-3.32 (m, 1H), 3.31-3.29 (m, 2H), 3.08-2.92 (m, 2H), 2.86 (s, 3H), 2.49-2.32 (m, 1H), 2.23-1.97 (m, 1H), 1.93-1.76 (m, 1H), 1.68-1.47 (m, 2H), 1.43 (d, J=6.4 Hz, 3H), 0.94 (t, J=7.2 Hz, 3H), 0.71 (t, J=7.2 Hz, 3H) ppm. MS: M/e 505 (M+1)⁺.

Compound A59: 2-(8-((2S,5R)-2,5-diethyl-4-(1-(5-fluorobenzo[d]thiazol-6-yl)ethyl)piperazin-1-yl)-5-methyl-6-oxo-5,6-dihydroimidazo[1,2-b]pyridazin-2-yl)acetonitrile

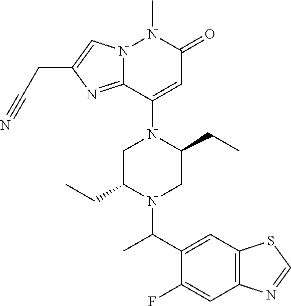 [SMILES: CC[C@H]1CN(C(C)c2cc3scnc3cc2F)[C@H](CC)CN1c1cc(=O)n(C)n2cc(CC#N)nc12]

### Step A: 6-bromo-5-fluorobenzo[d]thiazol-2-amine

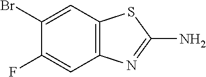 [SMILES: Nc1nc2cc(F)c(Br)cc2s1]

A mixture of 4-bromo-3-fluoroaniline (1 g, 5.3 mmol) and KSCN (760 mg, 7.9 mmol) in AcOH (3 mL) was added Br₂(0.85 g, 5.3 mmol) at 0° C., then warmed at room temperature for 2 hours. The solution was concentrated and diluted with water. The solution was concentrated and adjusted to pH=8-9 with saturated NaHCO₃ solution, extracted with ethyl acetate. The combined organic layers were purified by flash column chromatography to give the titled compound (800 mg, 61%). MS: M/e 247(M+1)⁺

### Step B: 6-bromo-5-fluorobenzo[d]thiazole

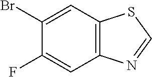 [SMILES: Fc1cc2ncsc2cc1Br]

A mixture of 6-bromo-5-fluorobenzo[d]thiazol-2-amine (800 mg, 3.2 mmol), tBuONO (474 mg, 14.8 mmol) in THF (10 mL) was stirred at 70° C. for 2 hours. The solution was concentrated and purified by flash column chromatography to give the titled compound (600 mg, 80%). MS: M/e 232(M+1)⁺

### Step C: 1-(5-fluorobenzo[d]thiazol-6-yl)ethan-1-one

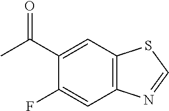

A mixture of 6-bromo-5-fluorobenzo[d]thiazole (300 mg, 1.25 mmol), tributyl(1-ethoxyvinyl)stannane (0.7 g, 1.9 mmol) and Pd(PPh₃)₂Cl₂ (90 mg, 0.12 mmol) in DMF (5 mL) was stirred at 100° C. under N₂ for 16 hours. The solution was diluted with ethyl acetate and washed with water. The organic layer was dried and added with HCl (3 mL, 3 M in ethyl acetate) in drops and the mixture was stirred at room temperature for 3 hours. The reaction was concentrated and adjusted to pH=8-9 with saturated NaHCO₃ solution, extracted with ethyl acetate. The combined organic layers were washed with brine, dried over Na₂SO₄ and the resulting residue was purified by flash column chromatography to give the titled compound (200 mg, 79%). MS: M/e 196(M+1)⁺

### Step D: 1-(5-fluorobenzo[d]thiazol-6-yl)ethan-1-ol

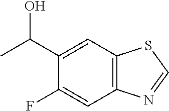

To a solution of 1-(5-fluorobenzo[d]thiazol-6-yl)ethan-1-one (200 mg, 1.02 mmol) in MeOH (3 mL) was added NaBH₄ (38 mg, 1.02 mmol) at room temperature and the resulting mixture was stirred at room temperature for 5 min. The reaction mixture was diluted with DCM and washed with water, dried over Na₂SO₄ and concentrated to give the titled compound (190 mg). MS: M/e 198 (M+1)⁺.

### Step E: 2-(8-((2S,5R)-2,5-diethyl-4-(1-(5-fluorobenzo[d]thiazol-6-yl)ethyl)piperazin-1-yl)-5-methyl-6-oxo-5,6-dihydroimidazo[1,2-b]pyridazin-2-yl)acetonitrile

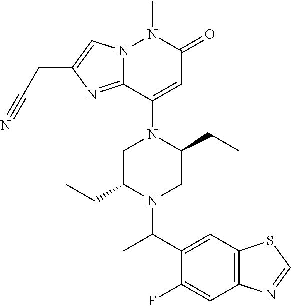 [SMILES: CC[C@H]1CN(C(C)c2cc3scnc3cc2F)[C@H](CC)CN1c1cc(=O)n(C)n2cc(CC#N)nc12]

To a solution of 2-(8-((2S,5R)-2,5-diethylpiperazin-1-yl)-5-methyl-6-oxo-5,6-dihydroimidazo[1,2-b]pyridazin-2-yl)acetonitrile (50 mg, 0.15 mmol) in CH₃CN (3 mL) and was added 1-(5-fluorobenzo[d]thiazol-6-yl)ethan-1-ol (82 mg, 0.45 mmol), (cyanomethyl)trimethylphosphonium iodide (111 mg, 0.45 mmol) and DIPEA (196 mg, 1.5 mmol). The resulting mixture was stirred at 105° C. overnight. The reaction solvent was removed under reduce pressure. The resulting residue was purified by flash column chromatography to give the titled Compound A59 (crude), which was further separated into Compound A59a (12 mg) and Compound A59b (9 mg) by Prep-HPLC(Method A).

Compound A59a (the earlier peak):¹H NMR (400 MHz, CD₃OD) δ 9.28 (s, 1H), 8.24 (d, J=6.6 Hz, 1H), 7.78 (t, J=5.4 Hz, 2H), 5.60 (s, 1H), 4.33 (q, J=6.5 Hz, 1H), 3.92 (s, 2H), 3.73 (s, 3H), 3.34-3.30 (m, 3H), 2.98 (d, J=2.9 Hz, 2H), 2.48 (s, 1H), 1.95 (ddt, J=20.6, 13.4, 6.7 Hz, 2H), 1.62-1.50 (m, 2H), 1.45 (d, J=6.5 Hz, 3H), 0.93 (t, J=7.3 Hz, 3H), 0.75 (t, J=7.3 Hz, 3H) ppm. MS: M/e 508 (M+1)⁺.

Compound A59b (the later peak): ¹H NMR (400 MHz, CD₃OD) δ 9.27 (s, 1H), 8.34 (d, J=6.6 Hz, 1H), 7.86-7.68 (m, 2H), 5.60 (s, 1H), 4.16 (q, J=6.7 Hz, 1H), 3.93 (s, 2H), 3.73 (s, 3H), 3.54 (d, J=12.1 Hz, 1H), 3.35-3.29 (m, 2H), 3.19 (d, J=9.9 Hz, 1H), 2.74 (d, J=8.8 Hz, 1H), 2.40 (d, J=12.3 Hz, 1H), 2.14-1.55 (m, 4H), 1.40 (d, J=6.5 Hz, 3H), 1.06 (t, J=7.2 Hz, 3H), 0.59 (t, J=7.3 Hz, 3H) ppm. MS: M/e 508 (M+1)⁺.

Compound A60: 2-(8-((2S,5R)-5-ethyl-4-(1-(5-fluoro-2-methylbenzo[d]thiazol-6-yl)ethyl)-2-methylpiperazin-1-yl)-5-methyl-6-oxo-5,6-dihydroimidazo[1,2-b]pyridazin-2-yl)acetonitrile

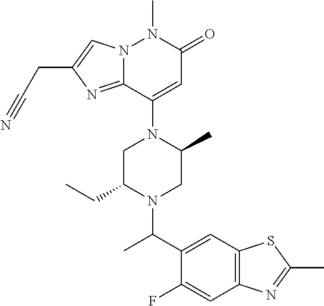 [SMILES: CC[C@@H]1CN(c2cc(=O)n(C)n3cc(CC#N)nc23)[C@@H](C)CN1C(C)c1cc2sc(C)nc2cc1F]

### Step A: N-(4-bromo-2,5-difluorophenyl)acetamide

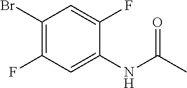

A mixture of 4-bromo-2,5-difluoroaniline (3 g, 14.5 mmol), Ac₂O (2.2 g, 21.7 mmol) in ethyl acetate (30 mL) was stirred at 50° C. under N₂ for 16 hours. The solution was diluted with ethyl acetate and washed with water. The organic layer was dried and purified by flash column chromatography to give the titled compound (3.3 g, 91%). MS: M/e 250(M+1)⁺

### Step B: N-(4-bromo-2,5-difluorophenyl)ethanethioamide

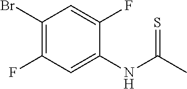

To a solution of N-(4-bromo-2,5-difluorophenyl)acetamide (3.3 g, 13.2 mmol) in toluene (30 mL) was added lawesson reagent (8 g, 19.8 mmol) and the resulting mixture was stirred at 110° C. for 16 hours. The reaction mixture was concentrated and purified by flash column chromatography to give the titled compound (3.6 g). MS: M/e 266 (M+1)⁺.

### Step C: 6-bromo-5-fluoro-2-methylbenzo[d]thiazole

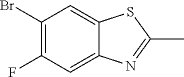

A mixture of N-(4-bromo-2,5-difluorophenyl)ethanethioamide (3 g, 11.2 mmol), NaOCH₃ (913 mg, 16.9 mmol) in NMP (3 mL) was stirred at 115° C. for 16 hours. The solution was diluted with ethyl acetate and washed with water. The organic layer was purified by flash column chromatography to give the titled compound (1.4 g, 50%). MS: M/e 246(M+1)⁺

### Step D: 1-(5-fluoro-2-methylbenzo[d]thiazol-6-yl)ethan-1-one

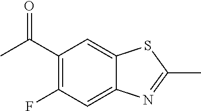

A mixture of 6-bromo-5-fluoro-2-methylbenzo[d]thiazole (1.4 g, 5.7 mmol), tributyl(1-ethoxyvinyl)stannane (3.1 g, 8.5 mmol) and Pd(PPh₃)₂Cl₂ (399 mg, 0.57 mmol) in toluene (20 mL) was stirred at 100° C. under N₂ for 16 hours. The solution was diluted with ethyl acetate and washed with water. The organic layer was dried and added with HCl (3 mL, 3 M in dioxane) in drops and the mixture was stirred at room temperature for 4 hours. The reaction was concentrated and adjusted to pH=8-9 with saturated NaHCO₃ solution, extracted with ethyl acetate. The combined organic layers were washed with brine, dried over Na₂SO₄ and the resulting residue was purified by flash column chromatography to give the titled compound (1 g, 84%). MS: M/e 210(M+1)⁺

### Step E: 1-(5-fluoro-2-methylbenzo[d]thiazol-6-yl)ethan-1-ol

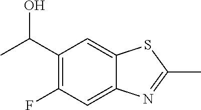

To a solution of 1-(5-fluoro-2-methylbenzo[d]thiazol-6-yl)ethan-1-one (1 g, 4.7 mmol) in MeOH (15 mL) was added NaBH₄ (180 mg, 4.7 mmol) at room temperature and the resulting mixture was stirred at room temperature for 5 min. The reaction mixture was diluted with DCM and washed with water, dried over Na₂SO₄, concentrated and purified by flash column chromatography to give the titled compound (900 mg, 90%). MS: M/e 212 (M+1)⁺.

### Step F: 2-(8-((2S,5R)-5-ethyl-4-(1-(5-fluoro-2-methylbenzo[d]thiazol-6-yl)ethyl)-2-methylpiperazin-1-yl)-5-methyl-6-oxo-5,6-dihydroimidazo[1,2-b]pyridazin-2-yl)acetonitrile

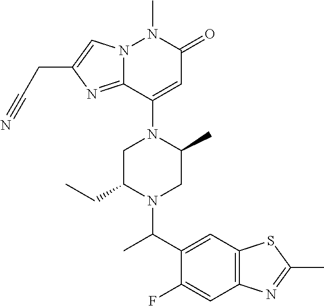 [SMILES: CC[C@@H]1CN(c2cc(=O)n(C)n3cc(CC#N)nc23)[C@@H](C)CN1C(C)c1cc2sc(C)nc2cc1F]

To a solution of 2-(8-((2S,5R)-5-ethyl-2-methylpiperazin-1-yl)-5-methyl-6-oxo-5,6-dihydroimidazo[1,2-b]pyridazin-2-yl)acetonitrile (50 mg, 0.16 mmol) in CH₃CN (3 mL) and was added 1-(5-fluoro-2-methylbenzo[d]thiazol-6-yl)ethan-1-ol (80 mg, 0.38 mmol), (cyanomethyl)trimethylphosphonium iodide (116 mg, 0.48 mmol) and DIPEA (207 mg, 1.5 mmol). The resulting mixture was stirred at 105° C. overnight. The reaction solvent was removed under reduce pressure. The resulting residue was purified by flash column chromatography to give the titled Compound A60 (crude), which was further separated into Compound A60a (7 mg) and Compound A60b (12 mg) by Prep-HPLC (Method A).

Compound A60a (the earlier peak):¹H NMR (400 MHz, CD₃OD) δ 8.10 (d, J=6.5 Hz, 1H), 7.78 (s, 1H), 7.60 (d, J=10.8 Hz, 1H), 5.62 (s, 1H), 5.13 (s, 1H), 4.84-4.57 (m, 1H), 4.32 (d, J=6.5 Hz, 1H), 3.92 (s, 2H), 3.73 (s, 3H), 3.05 (dd, J=11.9, 3.9 Hz, 1H), 2.83 (s, 3H), 2.78 (s, 1H), 2.47 (s, 2H), 1.68-1.52 (m, 2H), 1.40 (dd, J=20.8, 6.4 Hz, 6H), 0.80 (t, J=7.3 Hz, 3H) ppm. MS: M/e 508 (M+1)⁺.

Compound A60b (the later peak):¹H NMR (400 MHz, CD₃OD) δ 8.19 (d, J=6.6 Hz, 1H), 7.79 (s, 1H), 7.56 (d, J=10.9 Hz, 1H), 5.63 (s, 1H), 5.55-5.17 (m, 1H), 4.55 (s, 1H), 4.20-4.09 (m, 1H), 3.94 (s, 2H), 3.74 (s, 3H), 3.54 (d, J=12.2 Hz, 1H), 3.17 (d, J=9.9 Hz, 1H), 2.82 (s, 4H), 2.27 (d, J=11.7 Hz, 1H), 1.76-1.49 (m, 2H), 1.39 (d, J=6.5 Hz, 3H), 1.25 (d, J=6.4 Hz, 3H), 1.08 (t, J=7.2 Hz, 3H) ppm. MS: M/e 508 (M+1)⁺.

Compound A61: 2-(8-((2S,5R)-2,5-diethyl-4-(1-(5-fluoro-2-methylbenzo[d]thiazol-6-yl)ethyl)piperazin-1-yl)-5-methyl-6-oxo-5,6-dihydroimidazo[1,2-b]pyridazin-2-yl)acetonitrile

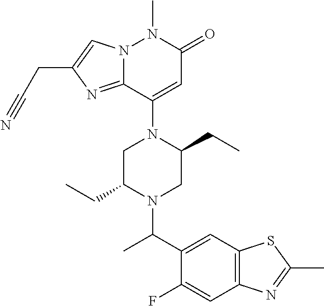 [SMILES: CC[C@H]1CN(C(C)c2cc3sc(C)nc3cc2F)[C@H](CC)CN1c1cc(=O)n(C)n2cc(CC#N)nc12]

To a solution of 2-(8-((2S,5R)-2,5-diethylpiperazin-1-yl)-5-methyl-6-oxo-5,6-dihydroimidazo[1,2-b]pyridazin-2-yl)acetonitrile (50 mg, 0.15 mmol) in CH₃CN (3 mL) and was added 1-(5-fluoro-2-methylbenzo[d]thiazol-6-yl)ethan-1-ol (80 mg, 0.38 mmol), (cyanomethyl)trimethylphosphonium iodide (111 mg, 0.45 mmol) and DIPEA (196 mg, 1.5 mmol). The resulting mixture was stirred at 105° C. overnight. The reaction solvent was removed under reduce pressure. The resulting residue was purified by flash column chromatography to give the titled Compound A61(crude), which was further separated into Compound A61a (8 mg) and Compound A61b (11 mg) by Prep-HPLC (Method A).

Compound A61a (the earlier peak): ¹H NMR (400 MHz, CD₃OD) δ 8.08 (d, J=6.5 Hz, 1H), 7.78 (s, 1H), 7.59 (d, J=10.8 Hz, 1H), 5.60 (s, 1H), 4.29 (q, J=6.3 Hz, 1H), 3.92 (s, 2H), 3.73 (s, 3H), 3.33-3.18 (m, 3H), 2.97 (s, 2H), 2.83 (s, 3H), 2.47 (s, 1H), 4.20-4.09 (m, 2H), 1.65-1.50 (m, 2H), 1.42 (d, J=6.5 Hz, 3H), 0.94 (t, J=7.2 Hz, 3H), 0.75 (t, J=7.2 Hz, 3H) ppm. MS: M/e 522 (M+1)⁺.

Compound A61b (the later peak): ¹H NMR (400 MHz, CD₃OD) δ 8.18 (d, J=6.6 Hz, 1H), 7.79 (s, 1H), 7.57 (d, J=10.9 Hz, 1H), 5.60 (s, 1H), 4.12 (d, J=6.2 Hz, 1H), 3.93 (s, 2H), 3.73 (s, 3H), 3.52 (d, J=12.2 Hz, 1H), 3.34-3.17 (m, 3H), 2.82 (s, 3H), 2.73 (d, J=10.5 Hz, 1H), 2.39 (d, J=12.3 Hz, 1H), 2.05 (s, 1H), 1.76-1.50 (m, 3H), 1.38 (d, J=6.5 Hz, 3H), 1.05 (t, J=7.1 Hz, 3H), 0.60 (t, J=7.0 Hz, 3H) ppm. MS: M/e 522 (M+1)⁺.

Compound A62: 2-(8-((2S,5R)-2,5-diethyl-4-(1-(7-fluorobenzo[d]thiazol-6-yl)ethyl)piperazin-1-yl)-5-methyl-6-oxo-5,6-dihydroimidazo[1,2-b]pyridazin-2-yl)acetonitrile

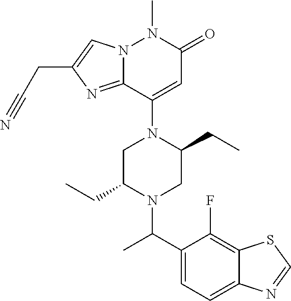 [SMILES: CC[C@H]1CN(C(C)c2ccc3ncsc3c2F)[C@H](CC)CN1c1cc(=O)n(C)n2cc(CC#N)nc12]

### Step A: N-((4-bromo-2,3-difluorophenyl)carbamothioyl)benzamide

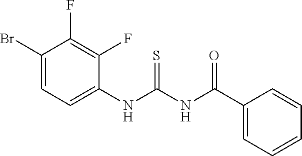 [SMILES: O=C(NC(=S)Nc1ccc(Br)c(F)c1F)c1ccccc1]

A solution of 4-bromo-2,3-difluoroaniline (2 g, 9.62 mmol) and benzoyl isothiocyanate (1.65 g, 10.12 mmol) in acetone (30 ml) was stirred at rt overnight. The reaction was filtered. The filter cake was dried to give the titled compound (2.53 g, 71%). MS: M/e 371,373 (M+1)⁺.

### Step B: 1-(4-bromo-2,3-difluorophenyl)thiourea

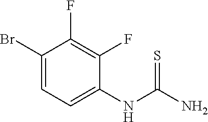 [SMILES: NC(=S)Nc1ccc(Br)c(F)c1F]

A solution of N-((4-bromo-2,3-difluorophenyl)carbamothioyl)benzamide (2.53 g, 6.84 mmol) and NaOH (816 mg, 20.4 mmol) in MeOH (25 ml) and water (5 ml) was stirred at 70° C. for 30 min. The reaction was concentrated under reduced pressure. The residue was filtered. The filter cake was dried to give the titled compound (0.83 g, 46%). MS: M/e 267,269 (M+1)⁺.

### Step C: 6-bromo-7-fluorobenzo[d]thiazol-2-amine

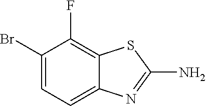 [SMILES: Nc1nc2ccc(Br)c(F)c2s1]

A solution of 1-(4-bromo-2,3-difluorophenyl)thiourea (830 mg, 3.12 mmol) and NaH (60%, 188 mg, 4.70 mmol) in NMP (10 ml) was stirred at 100° C. for 1 h. The reaction was quenched with water (20 ml) and extracted with EA (10 ml×2). The organic layer was dried over Na₂SO₄ and concentrated to dryness. The resulting residue was purified by flash column chromatography with 10-40% EA in PE to give the titled compound (760 mg, crude), which was used directly for the next step without further purification. MS: M/e 247,249 (M+1)⁺.

### Step D: 6-bromo-7-fluorobenzo[d]thiazole

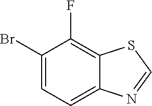 [SMILES: Fc1c(Br)ccc2ncsc12]

A solution of 6-bromo-7-fluorobenzo[d]thiazol-2-amine (760 mg, crude, 3.09 mmol) and tert-butyl nitrite (800 mg, 7.77 mmol) in THF (10 ml) was stirred at 70° C. for 2 h. The solution was quenched with water (10 ml) and extracted with EA (10 ml×2). The organic layer was dried over Na₂SO₄ and concentrated to dryness. The resulting residue was purified by flash column chromatography with 0-15% EtOAc in PE to give the titled compound (390 mg). MS: M/e 232,234 (M+1)⁺.

### Step E: 1-(7-fluorobenzo[d]thiazol-6-yl)ethan-1-one

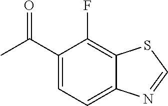

A solution of 6-bromo-7-fluorobenzo[d]thiazole (390 mg, 1.68 mmol), ethyl tributylstannanecarboxylate (910 mg, 2.52 mmol) and Pd(PPh₃)₂Cl₂ (59 mg, 0.084 mmol) in toluene (10 ml) was stirred at 100° C. overnight. The solution was cooled to RT. HCl/1,4-dioxane (4M, 2 ml) was added and stirred at RT for 10 min. The solution was diluted with EtOAc (15 ml), washed with aq. Na₂CO₃ (10 ml×2), dried over Na₂SO₄ and concentrated to dryness. The resulting residue was purified by flash column chromatography with 0-25% EtOAc in PE to give the titled compound (264 mg, 81%). MS: M/e 196 (M+1)⁺.

### Step F: 1-(7-fluorobenzo[d]thiazol-6-yl)ethan-1-ol

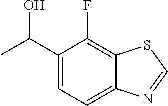

A solution of 1-(7-fluorobenzo[d]thiazol-6-yl)ethan-1-one (264 mg, 1.35 mmol) and NaBH₄ (51.4 mg, 1.35 mmol) in MeOH (4 ml) was stirred at RT for 15 min. The solution was diluted with EtOAc (10 ml), washed with brine (10 ml×2), dried over Na₂SO₄ and concentrated to dryness to give the titled compound (264 mg, 99%), which was used directly for the next step without further purification. MS: M/e 198 (M+1)⁺.

### Step G: 2-(8-((2S,5R)-2,5-diethyl-4-(1-(7-fluorobenzo[d]thiazol-6-yl)ethyl)piperazin-1-yl)-5-methyl-6-oxo-5,6-dihydroimidazo[1,2-b]pyridazin-2-yl)acetonitrile

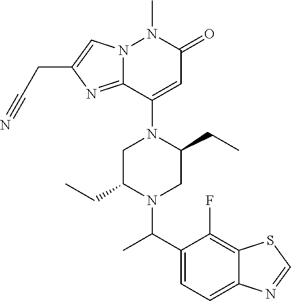 [SMILES: CC[C@H]1CN(C(C)c2ccc3ncsc3c2F)[C@H](CC)CN1c1cc(=O)n(C)n2cc(CC#N)nc12]

A solution of 2-(8-((2S,5R)-2,5-diethylpiperazin-1-yl)-5-methyl-6-oxo-5,6-dihydroimidazo[1,2-b]pyridazin-2-yl)acetonitrile (50 mg, 0.15 mmol), 1-(7-fluorobenzo[d]thiazol-6-yl)ethan-1-ol (60 mg, 0.30 mmol), (cyanomethyl)trimethylphosphonium iodide (111 mg, 0.46 mmol) and DIPEA (197.2 mg, 1.53 mmol) in CH₃CN (2 ml) was stirred at 100° C. overnight. The reaction was diluted with EtOAc (10 ml) and washed with brine (10 ml). The organic layer was concentrated under reduced pressure. The resulting residue was purified by flash column chromatography to give the titled Compound A62, which was further separated into Compound A62a (16.77 mg) and Compound A62b (21.17 mg) by Prep-HPLC(Method A).

Compound A62a (the earlier peak): 1H NMR (400 MHz, CD₃OD) δ 9.26 (s, 1H), 7.93 (d, J=8.0 Hz, 1H), 7.82-7.72 (m, 2H), 5.60 (s, 1H), 4.36 (q, J=6.5 Hz, 1H), 3.92 (s, 2H), 3.73 (s, 3H), 3.29-3.23 (m, 3H), 3.01 (q, J=12.0 Hz, 2H), 2.39 (s, 1H), 2.16-2.03 (m, 1H), 1.92-1.76 (m, 1H), 1.62-1.50 (m, 2H), 1.45 (d, J=5.7 Hz, 3H), 0.96 (t, J=8.0 Hz, 3H), 0.71 (t, J=8.0 Hz, 3H) ppm. MS: M/e 508 (M+1)⁺.

Compound A62b (the later peak): 1H NMR (400 MHz, CD₃OD) δ 9.25 (s, 1H), 7.91 (d, J=8.3 Hz, 1H), 7.84 (t, J=8.3 Hz, 1H), 7.78 (s, 1H), 5.60 (s, 1H), 4.21 (q, J=6.5 Hz, 1H), 3.93 (s, 2H), 3.73 (s, 3H), 3.52 (d, J=12.7 Hz, 1H), 3.32 (s, 2H), 3.21 (d, J=9.9 Hz, 1H), 2.75 (d, J=11.4 Hz, 1H), 2.35 (d, J=12.7 Hz, 1H), 1.97 (s, 1H), 1.78-1.51 (m, 3H), 1.41 (d, J=6.2 Hz, 3H), 1.06 (t, J=8.0 Hz, 3H), 0.59 (t, J=8.0 Hz, 3H) ppm. MS: M/e 508 (M+1)⁺.

Compound A63: 2-(8-((2S,5R)-5-ethyl-4-(1-(7-fluoro-2-methylbenzo[d]thiazol-6-yl)ethyl)-2-methylpiperazin-1-yl)-5-methyl-6-oxo-5,6-dihydroimidazo[1,2-b]pyridazin-2-yl)acetonitrile

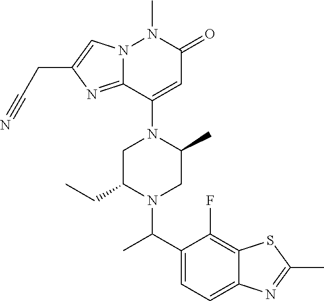 [SMILES: CC[C@@H]1CN(c2cc(=O)n(C)n3cc(CC#N)nc23)[C@@H](C)CN1C(C)c1ccc2nc(C)sc2c1F]

### Step A: N-(4-bromo-2,3-difluorophenyl)acetamide

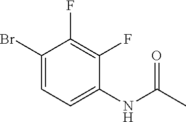

A solution of 4-bromo-2,3-difluoroaniline (2.7 g, 12.98 mmol) and acetic anhydride (2.7 ml) in EA (30 ml) was stirred at 50° C. for 1.5 h. The solution was washed with aq. Na₂CO₃ (20 ml×2), dried over Na₂SO₄ and concentrated to dryness to give the titled compound (3.2 g, 99%). MS: M/e 250,252 (M+1)⁺.

### Step B: N-(4-bromo-2,3-difluorophenyl)ethanethioamide

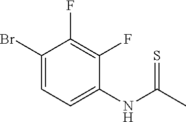

A solution of N-(4-bromo-2,3-difluorophenyl)acetamide (3 g, 12.05 mmol) and Lawesson's Reagent (2.43 g, 6.01 mmol) in toluene (30 ml) was stirred at 110° C. for 1.5 h. The solution was diluted with EtOAc (30 ml), washed with brine (10 ml×2), dried over Na₂SO₄ and concentrated to dryness. The resulting residue was purified by flash column chromatography with 0-15% EA in PE to give the titled compound (3 g, 94%). MS: M/e 266,268 (M+1)⁺.

### Step C: 6-bromo-7-fluoro-2-methylbenzo[d]thiazole

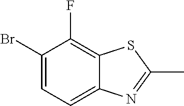

A solution of N-(4-bromo-2,3-difluorophenyl)ethanethioamide (3 g, 11.32 mmol) and Cs2CO3 (7.4 g, 22.70 mmol) in toluene (30 ml) was stirred at 110° C. for 1.5 h. The solution was diluted with EtOAc (30 ml), washed with brine (10 ml×2), dried over Na₂SO₄ and concentrated to dryness. The resulting residue was purified by flash column chromatography with 0-10% EA in PE to give the titled compound (2.1 g, 76%). MS: M/e 246,248 (M+1)⁺.

### Step D: 1-(7-fluoro-2-methylbenzo[d]thiazol-6-yl)ethan-1-one

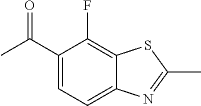

A solution of 6-bromo-7-fluoro-2-methylbenzo[d]thiazole (2.1 g, 8.57 mmol), ethyl tributylstannanecarboxylate (4.64 g, 12.85 mmol) and Pd(PPh₃)₂Cl₂ (300 mg, 0.43 mmol) in toluene (20 ml) was stirred at 100° C. overnight. The solution was cooled to RT. HCl/1,4-dioxane (4M, 2 ml) was added and stirred at RT for 10 min. The solution was diluted with EtOAc (20 ml), washed with aq. Na₂CO₃ (10 ml×2), dried over Na₂SO₄ and concentrated to dryness. The resulting residue was purified by flash column chromatography with 0-20% EA in PE to give the titled compound (1.7 g, 94%). MS: M/e 210 (M+1)⁺.

### Step E: 1-(7-fluoro-2-methylbenzo[d]thiazol-6-yl)ethan-1-ol

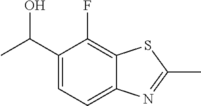

A solution of 1-(7-fluoro-2-methylbenzo[d]thiazol-6-yl)ethan-1-one (1.7 g, 8.13 mmol) and NaBH₄ (309 mg, 8.13 mmol) in MeOH (15 ml) was stirred at RT for 10 min. The solution was diluted with EtOAc (30 ml), washed with brine (10 ml×2), dried over Na₂SO₄ and concentrated to dryness to give the titled compound (1.7 g, 99%), which was used directly for the next step without further purification. MS: M/e 212 (M+1)⁺.

### Step F: 2-(8-((2S,5R)-5-ethyl-4-(1-(7-fluoro-2-methylbenzo[d]thiazol-6-yl)ethyl)-2-methylpiperazin-1-yl)-5-methyl-6-oxo-5,6-dihydroimidazo[1,2-b]pyridazin-2-yl)acetonitrile

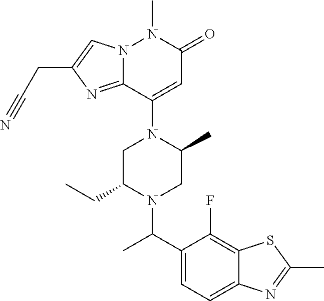 [SMILES: CC[C@@H]1CN(c2cc(=O)n(C)n3cc(CC#N)nc23)[C@@H](C)CN1C(C)c1ccc2nc(C)sc2c1F]

A solution of 2-(8-((2S,5R)-5-ethyl-2-methylpiperazin-1-yl)-5-methyl-6-oxo-5,6-dihydroimidazo[1,2-b]pyridazin-2-yl)acetonitrile (50 mg, 0.16 mmol), 1-(7-fluoro-2-methylbenzo[d]thiazol-6-yl)ethan-1-ol (67.2 mg, 0.32 mmol), (cyanomethyl)trimethylphosphonium iodide (116 mg, 0.48 mmol) and DIPEA (205 mg, 1.59 mmol) in CH₃CN (2 ml) was stirred at 100° C. overnight. The reaction was diluted with EtOAc (10 ml) and washed with brine (10 ml). The organic layer was concentrated under reduced pressure. The resulting residue was purified by flash column chromatography to give the titled Compound A63, which was further separated into Compound A63a (15 mg) and Compound A63b (19 mg) by Prep-HPLC(Method A).

Compound A63a (the earlier peak): ¹H NMR (400 MHz, CD₃OD) δ 7.80-7.73 (m, 2H), 7.70 (t, J=7.0 Hz, 1H), 5.62 (s, 1H), 5.40-5.25 (m, 1H), 4.51-4.48 (m, 1H), 4.33 (q, J=6.6 Hz, 1H), 3.91 (s, 2H), 3.73 (s, 3H), 3.27 (m, 1H), 3.04 (dd, J=11.99, 4.00 Hz, 1H), 2.90-2.79 (m, 4H), 2.40 (s, 1H), 1.61-1.51 (m, 2H), 1.43 (d, J=6.5 Hz, 3H), 1.39 (d, J=6.4 Hz, 3H), 0.77 (t, J=7.3 Hz, 3H) ppm. MS: M/e 508 (M+1)⁺.

Compound A63b (the later peak): ¹H NMR (400 MHz, CD₃OD) δ 7.82-7.75 (m, 2H), 7.72 (d, J=8.5 Hz, 1H), 5.62 (s, 1H), 5.15-5.00 (m, 1H), 4.82-4.75 (m, 1H), 4.18 (q, J=6.5 Hz, 1H), 3.93 (s, 2H), 3.73 (s, 3H), 3.52 (d, J=12.4 Hz, 1H), 3.18 (d, J=10.4 Hz, 1H), 2.90-2.78 (m, 4H), 2.22 (d, J=12.1 Hz, 1H), 1.80-1.65 (m, 1H), 1.64-1.52 (m, 1H), 1.40 (d, J=6.6 Hz, 3H), 1.23 (d, J=6.6 Hz, 3H), 1.09 (t, J=7.3 Hz, 3H) ppm. MS: M/e 508 (M+1)⁺.

Compound A64: 2-(8-((2S,5R)-2,5-diethyl-4-(1-(7-fluoro-2-methylbenzo[d]thiazol-6-yl)ethyl)piperazin-1-yl)-5-methyl-6-oxo-5,6-dihydroimidazo[1,2-b]pyridazin-2-yl)acetonitrile

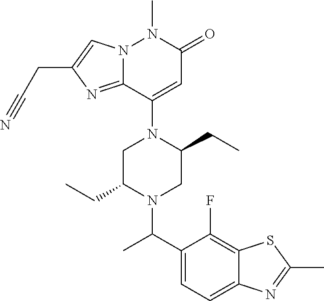 [SMILES: CC[C@H]1CN(C(C)c2ccc3nc(C)sc3c2F)[C@H](CC)CN1c1cc(=O)n(C)n2cc(CC#N)nc12]

A solution of 2-(8-((2S,5R)-2,5-diethylpiperazin-1-yl)-5-methyl-6-oxo-5,6-dihydroimidazo[1,2-b]pyridazin-2-yl)acetonitrile (100 mg, 0.30 mmol), 1-(7-fluoro-2-methylbenzo[d]thiazol-6-yl)ethan-1-ol (128.6 mg, 0.61 mmol), (cyanomethyl)trimethylphosphonium iodide (222.3 mg, 0.91 mmol) and DIPEA (393.3 mg, 3.04 mmol) in CH₃CN (3 ml) was stirred at 100° C. overnight. The reaction was diluted with EtOAc (10 ml) and washed with brine (10 ml). The organic layer was concentrated under reduced pressure. The resulting residue was purified by flash column chromatography to give the titled Compound A64, which was further separated into Compound A64a (28 mg) and Compound A64b (35 mg) by Prep-HPLC(Method A).

Compound A64a (the earlier peak): ¹H NMR (400 MHz, CD₃OD) δ 7.82-7.73 (m, 2H), 7.68 (t, J=7.3 Hz, 1H), 5.60 (s, 1H), 4.31 (q, J=6.4 Hz, 1H), 3.92 (s, 2H), 3.73 (s, 3H), 3.31 (s, 2H), 3.26 (s, 1H), 2.97 (q, J=12.7 Hz, 2H), 2.85 (s, 3H), 2.39 (s, 1H), 2.16-2.00 (m, 1H), 1.91-1.75 (m, 1H), 1.64-1.49 (m, 2H), 1.43 (d, J=6.4 Hz, 3H), 0.96 (t, J=7.2 Hz, 3H), 0.71 (t, J=7.1 Hz, 3H) ppm. MS: M/e 522 (M+1)⁺.

Compound A64b (the later peak): ¹H NMR (400 MHz, CD₃OD) δ 7.87-7.67 (m, 3H), 5.59 (s, 1H), 4.17 (q, J=6.4 Hz, 1H), 3.93 (s, 2H), 3.73 (s, 3H), 3.50 (d, J=13.1 Hz, 1H), 3.31 (s, 2H), 3.18 (d, J=9.8 Hz, 1H), 2.85 (s, 3H), 2.73 (d, J=10.1 Hz, 1H), 2.34 (d, J=12.2 Hz, 1H), 1.97 (s, 1H), 1.76-1.50 (m, 3H), 1.39 (d, J=6.5 Hz, 3H), 1.05 (t, J=7.1 Hz, 3H), 0.60 (t, J=7.1 Hz, 3H) ppm. MS: M/e 522 (M+1)⁺.

Compound A65: 2-(8-((2S,5R)-4-(1-(benzo[d]thiazol-5-yl)ethyl)-2,5-diethylpiperazin-1-yl)-5-methyl-6-oxo-5,6-dihydroimidazo[1,2-b]pyridazin-2-yl)acetonitrile

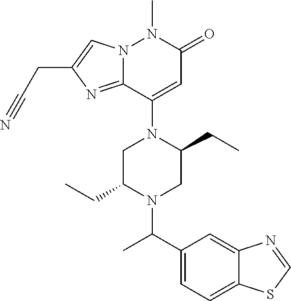 [SMILES: CC[C@H]1CN(C(C)c2ccc3scnc3c2)[C@H](CC)CN1c1cc(=O)n(C)n2cc(CC#N)nc12]

### Step A: 1-(benzo[d]thiazol-5-yl)ethan-1-one

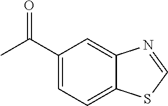

A mixture of 5-bromobenzo[d]thiazole (2 g mg, 9.3 mmol), tributyl(1-ethoxyvinyl)stannane (6.8 g, 18.6 mmol) and Pd(PPh₃)₂Cl₂ (660 mg, 0.9 mmol) in toluene (40 mL) was stirred at 100° C. under N₂ for 16 hours. The reaction mixture was quenched by HCl (10 mL, 4 M in 1,4-dioxane) in drops and the mixture was stirred at room temperature for 0.5 hour. The reaction mixture was concentrated. The resulting residue was purified by flash column chromatography to give the titled compound (600 mg, 36%). MS: M/e 178 (M+1)⁺.

### Step B: 1-(benzo[d]thiazol-5-yl)ethan-1-ol

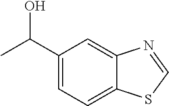

To a solution of 1-(benzo[d]thiazol-5-yl)ethan-1-one (600 mg, 3.4 mmol) in MeOH (30 mL) was added NaBH₄ (130 mg, 3.4 mmol) at room temperature and the resulting mixture was stirred at room temperature for 5 min. The reaction mixture was diluted with DCM and washed with water, dried over Na₂SO₄ and concentrated to give the titled compound (700 mg, crude). MS: M/e 180 (M+1)⁺.

### Step C: 2-(8-((2S,5R)-4-(1-(benzo[d]thiazol-5-yl)ethyl)-2,5-diethylpiperazin-1-yl)-5-methyl-6-oxo-5,6-dihydroimidazo[1,2-b]pyridazin-2-yl)acetonitrile

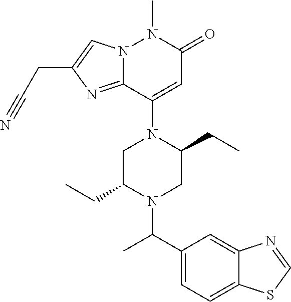 [SMILES: CC[C@H]1CN(C(C)c2ccc3scnc3c2)[C@H](CC)CN1c1cc(=O)n(C)n2cc(CC#N)nc12]

A mixture of 2-(8-((2S,5R)-2,5-diethylpiperazin-1-yl)-5-methyl-6-oxo-5,6-dihydroimidazo[1,2-b]pyridazin-2-yl)acetonitrile (328 mg, 1 mmol), 1-(benzo[d]thiazol-5-yl)ethan-1-ol (358 mg, 2 mmol), (cyanomethyl)trimethylphosphonium iodide (729 mg, 3 mmol) and DIPEA (645 mg, 5 mmol) in CH₃CN (10 mL) was stirred overnight at 100° C. in a sealed tube. The reaction mixture was diluted with EtOAc (15 mL), washed with H₂O, brine, dried over Na₂SO₄, concentrated to dryness. The resulting residue was purified by flash column chromatography to give the titled Compound A65 (320 mg), which was further separated into Compound A65a (70 mg) and Compound A65b (90 mg) by Prep-HPLC (Method B).

Compound A65: ¹H NMR (500 MHz, CD₃OD) δ 9.28-9.21 (m, 1H), 8.13-8.00 (m, 2H), 7.80-7.76 (m, 1H), 7.62-7.57 (m, 1H), 5.62-5.57 (m, 1H), 4.00-3.95 (m, 0.5H), 3.94-3.89 (m, 2H), 3.84-3.79 (m, 0.5H), 3.73 (s, 3H), 3.57-3.51 (m, 0.5H), 3.30-3.28 (m, 2.5H), 3.25-3.18 (m, 0.5H), 3.09-2.92 (m, 1H), 2.74-2.69 (m, 0.5H), 2.48-2.37 (m, 1H), 2.20-1.95 (m, 1H), 1.91-1.50 (m, 3H), 1.47-1.38 (m, 3H), 1.09-0.95 (m, 3H), 0.71-0.54 (m, 3H) ppm. MS: M/e 490 (M+1)⁺.

Compound A65a (the earlier peak): ¹H NMR (500 MHz, CD₃OD) δ 9.25 (s, 1H), 8.08-8.02 (m, 2H), 7.77 (s, 1H), 7.57 (d, J=80 Hz, 1H), 5.60 (s, 1H), 3.97-3.86 (m, 3H), 3.73 (s, 3H), 3.31-3.25 (m, 3H), 3.08-2.89 (m, 2H), 2.47-2.40 (m, 1H), 2.20-2.09 (m, 1H), 1.90-1.79 (m, 1H), 1.64-1.48 (m, 2H), 1.42 (d, J=6.0 Hz, 3H), 0.96 (t, J=6.8 Hz, 3H), 0.67 (t, J=6.8 Hz, 3H) ppm. MS: M/e 490 (M+1)⁺.

Compound A65b (the later peak): ¹H NMR (500 MHz, CD₃OD) δ 9.23 (s, 1H), 8.10 (s, 1H), 8.02 (d, J=8.4 Hz, 1H), 7.78 (s, 1H), 7.59 (d, J=8.4 Hz, 1H), 5.58 (s, 1H), 3.93 (s, 2H), 3.82-3.76 (m, 1H), 3.73 (s, 3H), 3.55-3.46 (m, 1H), 3.31-3.29 (m, 2H), 3.23-3.16 (m, 1H), 2.71-2.63 (m, 1H), 2.41-2.34 (m, 1H), 2.04-1.94 (m, 1H), 1.74-1.55 (m, 3H), 1.38 (d, J=6.0 Hz, 3H), 1.05 (t, J=6.8 Hz, 3H), 0.56 (t, J=6.8 Hz, 3H) ppm. MS: M/e 490 (M+1)⁺.

Compound A66: 2-(8-((2S,5R)-2,5-diethyl-4-(1-(4-fluorobenzo[d]thiazol-5-yl)ethyl)piperazin-1-yl)-5-methyl-6-oxo-5,6-dihydroimidazo[1,2-b]pyridazin-2-yl)acetonitrile

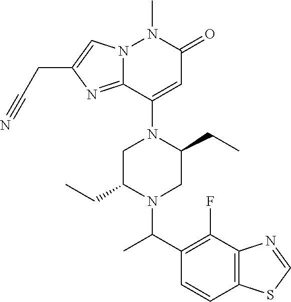 [SMILES: CC[C@H]1CN(C(C)c2ccc3scnc3c2F)[C@H](CC)CN1c1cc(=O)n(C)n2cc(CC#N)nc12]

### Step A: N-((3-bromo-2-fluorophenyl)carbamothioyl)benzamide

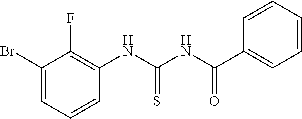 [SMILES: O=C(NC(=S)Nc1cccc(Br)c1F)c1ccccc1]

A solution of 3-bromo-2-fluoroaniline (5 g, 26.32 mmol) and benzoyl isothiocyanate (4.5 g, 27.61 mmol) in MeCN (50 ml) was stirred at rt for 30 min. The reaction was filtered. The filter cake was dried to give the titled compound (7.5 g, 81%). MS: M/e 353,355 (M+1)⁺.

### Step B: 1-(3-bromo-2-fluorophenyl)thiourea

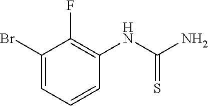 [SMILES: NC(=S)Nc1cccc(Br)c1F]

A solution of N-((3-bromo-2-fluorophenyl)carbamothioyl)benzamide (7.5 g, 21.31 mmol) and NaOH (2.56 g, 64 mmol) in MeOH (80 ml) and H2O (20 ml) was stirred at 70° C. for 15 min. The reaction was concentrated to dryness under reduced pressure. The resulting solid was slurried in DCM:MeOH (10:1, 50 ml) and then filtered. The filtrate was evaporated to give the product (4 g, 75%). MS: M/e 249,251 (M+1)⁺.

### Step C: 5-bromo-4-fluorobenzo[d]thiazol-2-amine

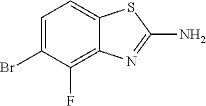 [SMILES: Nc1nc2c(F)c(Br)ccc2s1]

To a solution of 1-(3-bromo-2-fluorophenyl)thiourea (248 mg, 1 mol) in CHCl₃ (2 mL) was added Br₂ (192 mg, 1.2 mmol). The reaction mixture was stirred at 60° C. overnight. The mixture was slowly added H₂O (20 ml) and extracted with EA (20 mL×3). The organic phase was dried with Na₂SO₄ and concentrated by using a rotary evaporator to give a residue. The resulting residue was purified by flash column chromatography to give the titled compound (200 mg, 81.6%). MS: M/e 247 (M+1)⁺.

### Step D: 5-bromo-4-fluorobenzo[d]thiazole

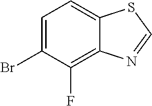 [SMILES: Fc1c(Br)ccc2scnc12]

To a solution of 5-bromo-4-fluorobenzo[d]thiazol-2-amine (110 mg, 0.45 mol) in THF (10 mL) was added Isopentyl nitrite (131 mg, 1.12 mmol). The reaction mixture was stirred at 70° C. overnight. The mixture was slowly added H₂O (20 ml) and extracted with EA (20 mL×3). The organic phase was dried with Na₂SO₄ and concentrated by using a rotary evaporator to give a residue. The resulting residue was purified by flash column chromatography to give the titled compound (60 mg, 58.3%). MS: M/e 232 (M+1)⁺.

### Step E: 1-(4-fluorobenzo[d]thiazol-5-yl)ethan-1-one

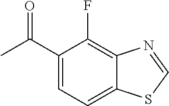

To a solution of 5-bromo-4-fluorobenzo[d]thiazole (150 mg, 0.65 mol) in toluene (10 mL) was added Pd(PPh₃)₂Cl₂ (70 mg, 0.1 mol) and tributyl(1-ethoxyvinyl)stannane (365 mg, 1 mmol). The reaction mixture was protected by N₂ atmosphere and stirred at 90° C. for 16 h. The mixture was added 4N Hcl in dionxane (0.5 ml) and stirred at rt for 15 mins. The mixture was added saturated NaHCO₃ aqueous solution (10 ml) and extracted with EA (20 mL×3). The organic phase was dried with Na₂SO₄ and concentrated by using a rotary evaporator to give a residue. The resulting residue was purified by flash column chromatography to give the titled compound (120 mg, 95.2%). MS: M/e 196 (M+1)⁺.

### Step F: 1-(4-fluorobenzo[d]thiazol-5-yl)ethan-1-ol

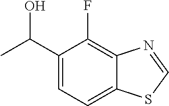

To a solution of 1-(4-fluorobenzo[d]thiazol-5-yl)ethan-1-one (120 mg, 0.65 mol) in MeOH was added NaBH₄ (23 mg, 0.6 mmol). The reaction mixture was stirred at rt for 15 mins. The mixture was added H₂O (20 ml) and extracted with DCM (20 mL×3). The organic phase was dried with Na₂SO₄ and concentrated by using a rotary evaporator to give a residue. The resulting residue was purified by flash column chromatography to give the titled compound (100 mg, 83.3%). MS: M/e 198 (M+1)⁺.

### Step G: 2-(8-((2S,5R)-2,5-diethyl-4-(1-(4-fluorobenzo[d]thiazol-5-yl)ethyl)piperazin-1-yl)-5-methyl-6-oxo-5,6-dihydroimidazo[1,2-b]pyridazin-2-yl)acetonitrile

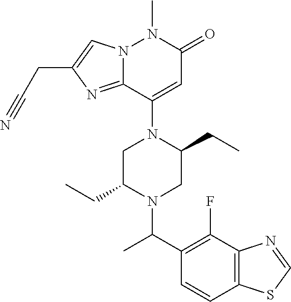 [SMILES: CC[C@H]1CN(C(C)c2ccc3scnc3c2F)[C@H](CC)CN1c1cc(=O)n(C)n2cc(CC#N)nc12]

To a solution of 2-(8-((2S,5R)-2,5-diethylpiperazin-1-yl)-5-methyl-6-oxo-5,6-dihydroimidazo[1,2-b]pyridazin-2-yl)acetonitrile (30 mg, 0.1 mmol), 1-(4-fluorobenzo[d]thiazol-5-yl)ethan-1-ol (50 mg, 0.25 mmol) and (cyanomethyl)trimethylphosphonium iodide (120 mg, 0.5 mmol) in CH₃CN (10 mL) was added DIPEA (258 mg, 2 mmol). The reaction mixture was sealed in a bottle and heated at 105° C. for 16 hours, and then cooled to room temperature, diluted with water, extracted with EtOAc (20 mL×3). The combined organic layers were washed with brine, dried over Na₂SO₄, filtered, and concentrated to dryness. The resulting residue was purified and separated into Compound A66a (4 mg) and Compound A66b (5 mg) by Prep-HPLC (Method A).

Compound A66a (the earlier peak): ¹H NMR (500 MHz, CD₃OD) δ 9.27 (s, 1H), 7.90 (d, J=8.3 Hz, 1H), 7.78 (s, 1H), 7.70 (m, 1H), 5.61 (s, 1H), 4.41 (d, J=6.4 Hz, 1H), 3.92 (s, 2H), 3.73 (s, 3H), 3.33 (s, 2H), 3.22 (m, 1H), 3.00 (dd, J=25.7, 10.2 Hz, 2H), 2.41 (d, J=7.8 Hz, 1H), 2.10 (m, 1H), 1.84 (m, 1H), 1.58 (d, J=6.9 Hz, 2H), 1.45 (d, J=6.3 Hz, 3H), 0.97 (t, J=6.8 Hz, 3H), 0.72 (t, J=6.9 Hz, 3H) ppm. MS: M/e 487 (M+1)⁺.

Compound A66b (the later peak): ¹H NMR (400 MHz, CD₃OD) δ 9.26 (s, 1H), 7.87 (d, J=8.5 Hz, 1H), 7.79 (m, 2H), 5.60 (s, 1H), 4.26 (d, J=6.4 Hz, 1H), 3.93 (s, 2H), 3.73 (s, 3H), 3.51 (d, J=12.1 Hz, 1H), 3.32 (s, 1H), 3.27 (m, 1H), 3.20 (d, J=9.3 Hz, 1H), 2.76 (d, J=10.2 Hz, 1H), 2.36 (d, J=12.2 Hz, 1H), 1.97 (s, 1H), 1.66 (m, 3H), 1.41 (d, J=6.5 Hz, 3H), 1.06 (t, J=7.0 Hz, 3H), 0.60 (t, J=7.3 Hz, 3H) ppm. MS: M/e 487 (M+1)⁺.

Compound A67: 8-((2S,5R)-4-(1-(benzo[d]thiazol-6-yl)ethyl)-2,5-diethylpiperazin-1-yl)-2-(but-2-yn-1-yl)-5-methylimidazo[1,2-b]pyridazin-6(5H)-one

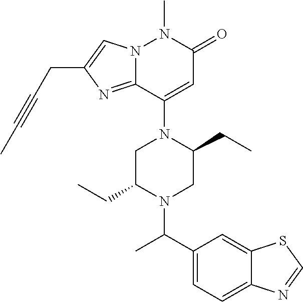 [SMILES: CC#CCc1cn2c(n1)c(N1C[C@@H](CC)N(C(C)c3ccc4ncsc4c3)C[C@@H]1CC)cc(=O)n2C]

### Step A: tert-butyl (2R,5S)-4-(2-(but-2-yn-1-yl)-5-methyl-6-oxo-5,6-dihydroimidazo[1,2-b]pyridazin-8-yl)-2,5-diethylpiperazine-1-carboxylate

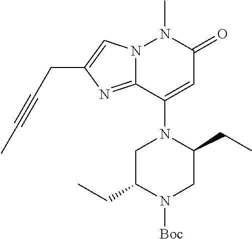 [SMILES: CC#CCc1cn2c(n1)c(N1C[C@@H](CC)N(C(=O)OC(C)(C)C)C[C@@H]1CC)cc(=O)n2C]

A mixture of tert-butyl (2R,5S)-4-(2-(chloromethyl)-5-methyl-6-oxo-5,6-dihydroimidazo[1,2-b]pyridazin-8-yl)-2,5-diethylpiperazine-1-carboxylate (407 mg, 0.93 mmol), but-2-ynoic acid (93 mg, 1.11 mmol), X-Phos (14.3 mmol), Pd(OAc)₂ (2.24 mg, 0.01 mmol) and Cs₂CO₃ (364 mg, 1.11 mmol) in THF (10 mL) was stirred at 80° C. overnight under N₂. The reaction mixture was treated with H₂O (20 mL), then extracted with EtOAc (15 mL×3). The combined organic layers were washed with brine, dried over Na₂SO₄, concentrated and purified by flash column chromatography to give the titled compound (233 mg, 56.8%). MS: M/e 442 (M+1)⁺.

### Step B: 2-(but-2-yn-1-yl)-8-((2S,5R)-2,5-diethylpiperazin-1-yl)-5-methylimidazo[1,2-b]pyridazin-6(5H)-one

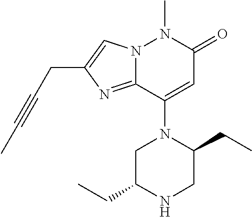 [SMILES: CC#CCc1cn2c(n1)c(N1C[C@@H](CC)NC[C@@H]1CC)cc(=O)n2C]

To a stirred solution of tert-butyl (2R,5S)-4-(2-(but-2-yn-1-yl)-5-methyl-6-oxo-5,6-dihydroimidazo[1,2-b]pyridazin-8-yl)-2,5-diethylpiperazine-1-carboxylate (233 mg, 0.53 mmol) in CH₂Cl₂ (5 mL) was added TFA (2 mL). After the addition, the reaction was stirred overnight. The reaction mixture was concentrated to give the residue, which was treated with H₂O (10 mL), then basified to pH=10˜11 and extracted with CH₂Cl₂ (10 mL×3). The combined organic layers were dried over Na₂SO₄, concentrated to give the titled compound (170 mg, 94.4%). MS: M/e 342 (M+1)⁺.

### Step C: 8-((2S,5R)-4-(1-(benzo[d]thiazol-6-yl)ethyl)-2,5-diethylpiperazin-1-yl)-2-(but-2-yn-1-yl)-5-methylimidazo[1,2-b]pyridazin-6(5H)-one

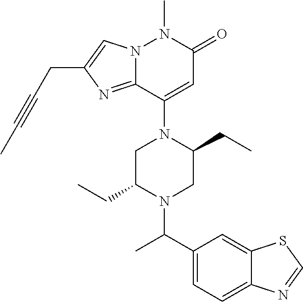 [SMILES: CC#CCc1cn2c(n1)c(N1C[C@@H](CC)N(C(C)c3ccc4ncsc4c3)C[C@@H]1CC)cc(=O)n2C]

A mixture of 2-(but-2-yn-1-yl)-8-((2S,5R)-2,5-diethylpiperazin-1-yl)-5-methylimidazo[1,2-b]pyridazin-6(5H)-one (34.1 mg, 0.1 mmol), 1-(benzo[d]thiazol-6-yl)ethan-1-ol (35.8 mg, 0.2 mmol), (cyanomethyl)trimethylphosphonium iodide (72.9 mg, 0.3 mmol) and DIPEA (129 mg, 1 mmol) in CH₃CN (4 mL) was stirred overnight at 100° C. in a sealed tube. The reaction mixture was diluted with EtOAc (15 mL), washed with H₂O, brine, dried over Na₂SO₄, concentrated to dryness. The resulting residue was purified by Pre-TLC (CH₂Cl₂/MeOH=10:1) to give the titled Compound A67, which was further separated into Compound A67a (4 mg) and Compound A67b (5 mg) by Prep-HPLC (Method B).

Compound A67a (the earlier peak): ¹H NMR (500 MHz, CD₃OD) δ 9.23 (s, 1H), 8.13-8.04 (m, 2H), 7.67-7.61 (m, 2H), 5.60 (s, 1H), 3.96 (dd, J=12.4, 6.4 Hz, 1H), 3.76 (s, 3H), 3.57-3.52 (m, 2H), 3.33-3.29 (m, 3H), 3.08-2.94 (m, 2H), 2.49-2.42 (m, 1H), 2.20-2.09 (m, 1H), 1.93-1.83 (m, 1H), 1.82-1.77 (m, 3H), 1.65-1.50 (m, 2H), 1.44 (d, J=6.4 Hz, 3H), 0.97 (t, J=7.2 Hz, 3H), 0.68 (t, J=7.2 Hz, 3H) ppm. MS: M/e 503(M+1)⁺.

Compound A67b (the later peak): ¹H NMR (500 MHz, CD₃OD) δ 9.22 (s, 1H), 8.12 (s, 1H), 8.04 (d, J=8.4 Hz, 1H), 7.69-7.63 (m, 2H), 5.59 (s, 1H), 3.81 (dd, J=12.8, 6.4 Hz, 1H), 3.76 (s, 3H), 3.58-3.50 (m, 3H), 3.33-3.31 (m, 2H), 3.24-3.18 (m, 1H), 2.75-2.69 (m, 1H), 2.41-2.34 (m, 1H), 2.05-1.94 (m, 1H), 1.81 (t, J=2.4 Hz, 3H), 1.74-1.57 (m, 3H), 1.40 (d, J=6.4 Hz, 3H), 1.05 (t, J=7.2 Hz, 3H), 0.58 (t, J=7.2 Hz, 3H) ppm. MS: M/e 503(M+1)⁺.

Compound A68: 2-(but-2-yn-1-yl)-8-((2S,5R)-2,5-diethyl-4-(1-(2-methylbenzo[d]thiazol-6-yl)ethyl)piperazin-1-yl)-5-methylimidazo[1,2-b]pyridazin-6(5H)-one

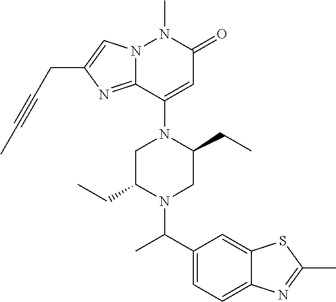 [SMILES: CC#CCc1cn2c(n1)c(N1C[C@@H](CC)N(C(C)c3ccc4nc(C)sc4c3)C[C@@H]1CC)cc(=O)n2C]

A mixture of 2-(but-2-yn-1-yl)-8-((2S,5R)-2,5-diethylpiperazin-1-yl)-5-methylimidazo[1,2-b]pyridazin-6(5H)-one (34.1 mg, 0.1 mmol), 1-(2-methylbenzo[d]thiazol-6-yl)ethan-1-ol (38.6 mg, 0.2 mmol), (cyanomethyl)trimethylphosphonium iodide (72.9 mg, 0.3 mmol) and DIPEA (64.5 mg, 0.5 mmol) in CH₃CN (4 mL) was stirred overnight at 100° C. in a sealed tube. The reaction mixture was diluted with EtOAc (10 mL), washed with H₂O, brine, dried over Na₂SO₄, concentrated to dryness. The resulting titled Compound A68 (crude) was purified and separated into Compound A68a (1.5 mg) and Compound A68b (2 mg) by Prep-HPLC (Method B).

Compound A68a (the earlier peak): ¹H NMR (400 MHz, CD₃OD) δ 7.91 (s, 1H), 7.85 (d, J=8.4 Hz, 1H), 7.62 (s, 1H), 7.53 (d, J=8.4 Hz, 1H), 5.57 (s, 1H), 3.88 (q, J=6.4 Hz, 1H), 3.73 (s, 3H), 3.55-3.48 (m, 2H), 3.35-3.31 (m, 2H), 3.29-3.24 (m, 1H), 3.05-2.88 (m, 2H), 2.82 (s, 3H), 2.46-2.38 (m, 1H), 2.15-2.04 (m, 1H), 1.90-1.80 (m, 1H), 1.78 (t, J=2.4 Hz, 3H), 1.59-1.48 (m, 2H), 1.39 (d, J=6.4 Hz, 3H), 0.94 (t, J=7.2 Hz, 3H), 0.65 (t, J=7.2 Hz, 3H) ppm. MS: M/e 517(M+1)⁺.

Compound A68b (the later peak): ¹H NMR (400 MHz, CD₃OD) δ 7.95 (s, 1H), 7.82 (d, J=8.4 Hz, 1H), 7.62 (s, 1H), 7.55 (d, J=8.4 Hz, 1H), 5.56 (s, 1H), 3.78-3.69 (m, 4H), 3.56-3.45 (m, 3H), 3.33-3.31 (m, 2H), 3.22-3.11 (m, 1H), 2.82 (s, 3H), 2.72-2.62 (m, 1H), 2.41-2.30 (m, 1H), 2.04-1.89 (m, 1H), 1.78 (s, 3H), 1.72-1.52 (m, 3H), 1.36 (d, J=6.4 Hz, 3H), 1.02 (t, J=7.2 Hz, 3H), 0.56 (t, J=7.2 Hz, 3H) ppm. MS: M/e 517(M+1)⁺.

Compound A69: 2-(but-2-yn-1-yl)-8-((2S,5R)-5-ethyl-2-methyl-4-(1-(2-methylbenzo[d]thiazol-6-yl)ethyl)piperazin-1-yl)-5-methylimidazo[1,2-b]pyridazin-6(5H)-one

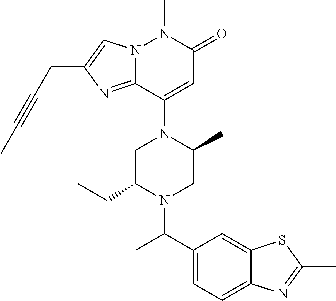 [SMILES: CC#CCc1cn2c(n1)c(N1C[C@@H](CC)N(C(C)c3ccc4nc(C)sc4c3)C[C@@H]1C)cc(=O)n2C]

### Step A: tert-butyl (2R,5S)-4-(2-(but-2-yn-1-yl)-5-methyl-6-oxo-5,6-dihydroimidazo[1,2-b]pyridazin-8-yl)-2-ethyl-5-methylpiperazine-1-carboxylate

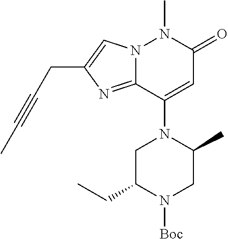 [SMILES: CC#CCc1cn2c(n1)c(N1C[C@@H](CC)N(C(=O)OC(C)(C)C)C[C@@H]1C)cc(=O)n2C]

A mixture of tert-butyl (2R,5S)-4-(2-(chloromethyl)-5-methyl-6-oxo-5,6-dihydroimidazo[1,2-b]pyridazin-8-yl)-2-ethyl-5-methylpiperazine-1-carboxylate (3 g, 7.08 mmol), but-2-ynoic acid (0.89 g, 10.62 mmol), X-Phos (101 mg, 0.212 mmol), Pd(OAc)₂ (15.7 mg, 0.07 mmol) and Cs₂CO₃ (3.46 g, 10.62 mmol) in THF (60 mL) was stirred at 80° C. overnight under N₂. The reaction mixture was treated with H₂O (20 mL), then extracted with EtOAc (100 mL). The organic layer was washed with brine, dried over Na₂SO₄, concentrated and purified by flash column chromatography to give the titled compound (1.9 g, 62.8%). MS: M/e 428 (M+1)⁺.

### Step B: 2-(but-2-yn-1-yl)-8-((2S,5R)-5-ethyl-2-methylpiperazin-1-yl)-5-methylimidazo[1,2-b]pyridazin-6(5H)-one

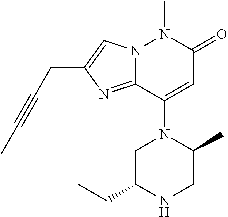 [SMILES: CC#CCc1cn2c(n1)c(N1C[C@@H](CC)NC[C@@H]1C)cc(=O)n2C]

To a stirred solution of tert-butyl (2R,5S)-4-(2-(but-2-yn-1-yl)-5-methyl-6-oxo-5,6-dihydroimidazo[1,2-b]pyridazin-8-yl)-2-ethyl-5-methylpiperazine-1-carboxylate (1.9 g, 4.44 mmol) in CH₂Cl₂ (30 mL) was added TMSOTf (1.97 g, 8.88 mmol). After the addition, the reaction was stirred for 20 min. The reaction mixture was quenched with aq.NaHCO₃, then extracted with CH₂Cl₂/IPA (3/1, 40 mL×3). The combined organic layers were dried over Na₂SO₄, concentrated to give the titled compound (1.5 g, 100%). MS: M/e 328 (M+1)⁺.

### Step C: 2-(but-2-yn-1-yl)-8-((2S,5R)-5-ethyl-2-methyl-4-(1-(2-methylbenzo[d]thiazol-6-yl)ethyl)piperazin-1-yl)-5-methylimidazo[1,2-b]pyridazin-6(5H)-one

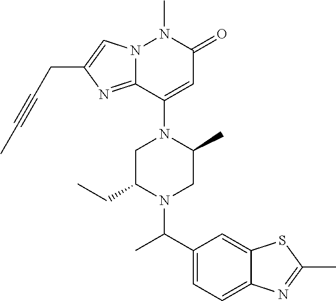 [SMILES: CC#CCc1cn2c(n1)c(N1C[C@@H](CC)N(C(C)c3ccc4nc(C)sc4c3)C[C@@H]1C)cc(=O)n2C]

A mixture of 2-(but-2-yn-1-yl)-8-((2S,5R)-5-ethyl-2-methylpiperazin-1-yl)-5-methylimidazo[1,2-b]pyridazin-6(5H)-one (50 mg, 0.153 mmol), 1-(2-methylbenzo[d]thiazol-6-yl)ethan-1-ol (59 mg, 0.31 mmol), (cyanomethyl)trimethylphosphonium iodide (111 mg, 0.46 mmol) and DIPEA (97 mg, 0.76 mmol) in CH₃CN (4 mL) was stirred overnight at 100° C. in a sealed tube. The reaction mixture was diluted with EtOAc (15 mL), washed with H₂O, brine, dried over Na₂SO₄, concentrated to dryness. The resulting residue was purified by flash column chromatography to give the titled compound (25 mg). ¹H NMR (400 MHz, CD₃OD) δ 7.98-7.90 (m, 1H), 7.89-7.79 (m, 1H), 7.64-7.51 (m, 2H), 5.59 (s, 1H), 3.93-3.76 (m, 1H), 3.73 (s, 3H), 3.58-3.46 (m, 2.5H), 3.30-3.24 (m, 1.5H), 3.21-2.95 (m, 1H), 2.90-2.72 (m, 4H), 2.47-2.19 (m, 1H), 1.82-1.75 (m, 3H), 1.74-1.46 (m, 3H), 1.43-1.16 (m, 6H), 1.10-0.67 (m, 3H) ppm. MS: M/e 503(M+1)⁺.

Compound A70: 2-(8-((2S,5R)-4-(1-(6-(2,2-difluorocyclopropyl)pyridin-3-yl)ethyl)-2,5-diethylpiperazin-1-yl)-5-methyl-6-oxo-5,6-dihydroimidazo[1,2-b]pyridazin-2-yl)acetonitrile

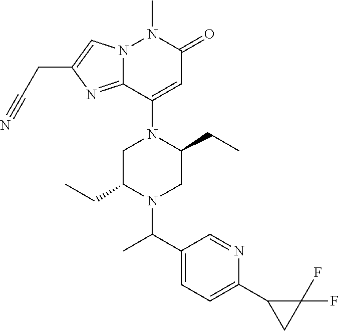 [SMILES: CC[C@H]1CN(C(C)c2ccc(C3CC3(F)F)nc2)[C@H](CC)CN1c1cc(=O)n(C)n2cc(CC#N)nc12]

### Step A: 5-bromo-2-vinylpyridine

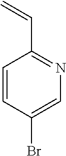

To a solution of 2,5-dibromopyridine (4.74 g, 20 mol) in Dioxane (40 mL) was added 4,4,5,5-tetramethyl-2-vinyl-1,3,2-dioxaborolane (3.4 g, 22 mmol), Pd(PPh₃)₄(2.3 g, 2 mmol), Na₂CO₃ (4.24 g, 40 mmol) and H₂O (10 ml). The reaction mixture was protected by N₂ atmosphere and stirred at 100° C. overnight. The mixture was added H₂O (20 ml) and extracted with EA (20 mL×3). The organic phase was dried with Na₂SO₄ and concentrated by using a rotary evaporator to give a residue. The resulting residue was purified by flash column chromatography to give the titled compound (1.3 g, 36%). MS: M/e 184 (M+1)⁺.

### Step B: 5-bromo-2-(2,2-difluorocyclopropyl)pyridine

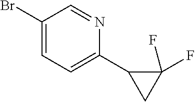 [SMILES: FC1(F)CC1c1ccc(Br)cn1]

To a solution of 5-bromo-2-vinylpyridine (366 mg, 2 mol) in THF (5 mL) was added TMSCF₃ (852 mg, 6 mmol) and NaI (300 mg, 2 mmol). The reaction mixture was stirred at 70° C. overnight. The mixture was added H₂O (20 ml) and extracted with EA (20 mL×3). The organic phase was dried with Na₂SO₄ and concentrated by using a rotary evaporator to give a residue. The resulting residue was purified by flash column chromatography to give the titled compound (140 mg, 30%). MS: M/e 234 (M+1)⁺.

### Step C: 1-(6-(2,2-difluorocyclopropyl)pyridin-3-yl)ethan-1-one

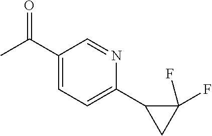

To a solution of 5-bromo-2-(2,2-difluorocyclopropyl)pyridine (140 mg, 0.6 mol) in toluene (10 mL) was added Pd(PPh₃)₂Cl₂ (70 mg, 0.1 mol) and tributyl(1-ethoxyvinyl)stannane (365 mg, 1 mmol). The reaction mixture was protected by N₂ atmosphere and stirred at 90° C. for 16 hrs. The mixture was added 4N HCl in dionxane (0.5 ml) and stirred at RT for 15 mins. The mixture was added saturated NaHCO₃ aqueous solution (10 ml) and extracted with EtOAc (20 mL×3). The organic phase was dried with Na₂SO₄ and concentrated by using a rotary evaporator to give a residue. The resulting residue was purified by flash column chromatography to give the titled compound (60 mg, 51%). MS: M/e 197 (M+1)⁺.

### Step D: 1-(6-(2,2-difluorocyclopropyl)pyridin-3-yl)ethan-1-ol

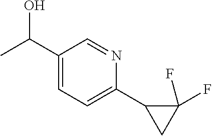

To a solution of 1-(6-(2,2-difluorocyclopropyl)pyridin-3-yl)ethan-1-one (60 mg, 0.3 mol) in MeOH was added NaBH₄ (23 mg, 0.6 mmol). The reaction mixture was stirred at RT for 15 mins. The mixture was added H₂O (20 ml) and extracted with DCM (20 mL×3). The organic phase was dried with Na₂SO₄ and concentrated by using a rotary evaporator to give a residue. The resulting residue was purified by flash column chromatography to give the titled compound (30 mg, 50%). MS: M/e 199 (M+1)⁺.

### Step E: 2-(8-((2S,5R)-4-(1-(6-(2,2-difluorocyclopropyl)pyridin-3-yl)ethyl)-2,5-diethylpiperazin-1-yl)-5-methyl-6-oxo-5,6-dihydroimidazo [1,2-b]pyridazin-2-yl)acetonitrile

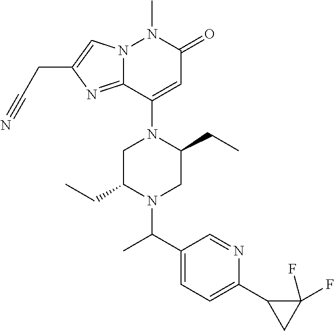 [SMILES: CC[C@H]1CN(C(C)c2ccc(C3CC3(F)F)nc2)[C@H](CC)CN1c1cc(=O)n(C)n2cc(CC#N)nc12]

To a solution of 2-(8-((2S,5R)-2,5-diethylpiperazin-1-yl)-5-methyl-6-oxo-5,6-dihydroimidazo[1,2-b]pyridazin-2-yl)acetonitrile (30 mg, 0.1 mmol), 1-(6-(2,2-difluorocyclopropyl)pyridin-3-yl)ethan-1-ol (30 mg, 0.15 mmol) and (cyanomethyl)trimethylphosphonium iodide (120 mg, 0.5 mmol) in CH₃CN (10 mL) was added DIPEA (258 mg, 2 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue contained the titled Compound A70 was purified and separated into Compound A70a (4 mg) and Compound A70b (4 mg) by Prep-HPLC(Method A).

Compound A70a (the earlier peak): ¹H NMR (500 MHz, CD₃OD) δ 8.46 (s, 1H), 7.83 (d, J=7.5 Hz, 1H), 7.78 (s, 1H), 7.38 (d, J=8.3 Hz, 1H), 5.61 (s, 1H), 3.93 (s, 2H), 3.85 (d, J=5.9 Hz, 1H), 3.73 (s, 3H), 3.22 (m, 3H), 3.03 (dd, J=24.7, 12.2 Hz, 2H), 2.91 (d, J=10.6 Hz, 1H), 2.37 (s, 1H), 2.12 (s, 2H), 1.88 (d, J=44.9 Hz, 2H), 1.54 (s, 2H), 1.38 (d, J=5.9 Hz, 3H), 0.94 (s, 3H), 0.73 (s, 3H) ppm. MS: M/e 510 (M+1)⁺.

Compound A70b (the later peak): ¹H NMR (400 MHz, CD₃OD) δ 8.49 (s, 1H), 7.84 (d, J=8.0 Hz, 1H), 7.79 (s, 1H), 7.36 (d, J=7.7 Hz, 1H), 5.60 (s, 1H), 3.94 (s, 2H), 3.71 (m, 4H), 3.49 (d, J=13.0 Hz, 1H), 3.33 (s, 2H), 3.16 (d, J=10.7 Hz, 1H), 3.02 (d, J=9.2 Hz, 1H), 2.70 (d, J=11.9 Hz, 1H), 2.30 (d, J=12.1 Hz, 1H), 2.12 (s, 1H), 1.92 (s, 2H), 1.63 (m, 3H), 1.35 (d, J=5.8 Hz, 3H), 1.04 (d, J=6.8 Hz, 3H), 0.61 (d, J=6.6 Hz, 3H) ppm. MS: M/e 510 (M+1)⁺.

Compound A71: 2-(8-((2S,5R)-4-(1-(benzo[d][1,3]dioxol-5-yl)ethyl)-2,5-diethylpiperazin-1-yl)-5-methyl-6-oxo-5,6-dihydroimidazo[1,2-b]pyridazin-2-yl)acetonitrile

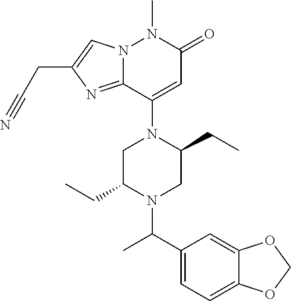 [SMILES: CC[C@H]1CN(C(C)c2ccc3c(c2)OCO3)[C@H](CC)CN1c1cc(=O)n(C)n2cc(CC#N)nc12]

To a solution of 2-(8-((2S,5R)-2,5-diethylpiperazin-1-yl)-5-methyl-6-oxo-5,6-dihydroimidazo[1,2-b]pyridazin-2-yl)acetonitrile (100 mg, 0.30 mmol) in CH₃CN (3 mL) and was added 1-(benzo[d][1,3]dioxol-5-yl)ethan-1-ol (51 mg, 0.30 mmol), (cyanomethyl)trimethylphosphonium iodide (223 mg, 0.91 mmol) and DIPEA (197 mg, 1.52 mmol). The resulting mixture was stirred at 105° C. overnight. The reaction solvent was removed under reduce pressure to afford crude product. The resulting residue was purified by Prep-HPLC(Method A) to give the titled compound (34 mg, 23%).¹H NMR (400 MHz, CD₃OD) δ 7.78 (s, 1H), 6.90 (d, J=14.4 Hz, 1H), 6.86-6.71 (m, 2H), 5.92 (d, J=8.5 Hz, 2H), 5.60 (s, 1H), 3.93 (s, 2H), 3.73 (s, 3H), 3.70-3.52 (m, 1H), 3.51-3.32 (m, 1H), 3.25 (s, 2H), 3.14-2.95 (m, 1H), 2.89-2.59 (m, 1H), 2.52-2.39 (m, 1H), 2.13-1.91 (m, 1H), 1.84-1.46 (m, 3H), 1.29 (dd, J=13.5, 6.2 Hz, 3H), 0.98 (dt, J=26.8, 7.0 Hz, 3H), 0.69 (dt, J=22.5, 7.0 Hz, 3H) ppm. MS: M/e 477(M+1)⁺.

Compound A72: 2-(8-((2S,5R)-2,5-diethyl-4-(1-(4-methoxyphenyl)ethyl)piperazin-1-yl)-5-methyl-6-oxo-5,6-dihydroimidazo[1,2-b]pyridazin-2-yl)acetonitrile

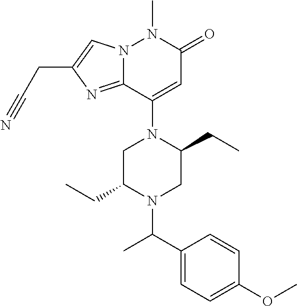 [SMILES: CC[C@H]1CN(C(C)c2ccc(OC)cc2)[C@H](CC)CN1c1cc(=O)n(C)n2cc(CC#N)nc12]

To a solution of 2-(8-((2S,5R)-2,5-diethylpiperazin-1-yl)-5-methyl-6-oxo-5,6-dihydroimidazo[1,2-b]pyridazin-2-yl)acetonitrile (100 mg, 0.30 mmol) in CH₃CN (3 mL) and was added 1-(4-methoxyphenyl)ethan-1-ol (46 mg, 0.30 mmol), (cyanomethyl)trimethylphosphonium iodide (223 mg, 0.91 mmol) and DIPEA (197 mg, 1.52 mmol). The resulting mixture was stirred at 105° C. overnight. The reaction solvent was removed under reduce pressure. The resulting residue was purified by Prep-HPLC(Method A) to give the titled compound (38 mg, 27%). ¹H NMR (400 MHz, CD₃OD) δ 7.78 (s, 1H), 7.27 (t, J=9.2 Hz, 2H), 6.88 (t, J=9.2 Hz, 2H), 5.59 (s, 1H), 3.93 (s, 2H), 3.78 (d, J=4.8 Hz, 3H), 3.73 (s, 3H), 3.69-3.53 (m, 1H), 3.49-3.33 (m, 1H), 3.24 (s, 2H), 3.15-2.95 (m, 1H), 2.88-2.58 (m, 1H), 2.49-2.36 (m, 1H), 2.11-1.90 (m, 1H), 1.83-1.46 (m, 3H), 1.31 (dd, J=16.2, 6.0 Hz, 3H), 0.98 (dt, J=14.7, 7.0 Hz, 3H), 0.75-0.59 (m, 3H). MS: M/e 463(M+1)⁺.

Compound A73: 2-(8-((2S,5R)-4-(1-(6-cyclopropylpyridin-3-yl)ethyl)-2,5-diethylpiperazin-1-yl)-7-fluoro-5-methyl-6-oxo-5,6-dihydroimidazo[1,2-b]pyridazin-2-yl)acetonitrile

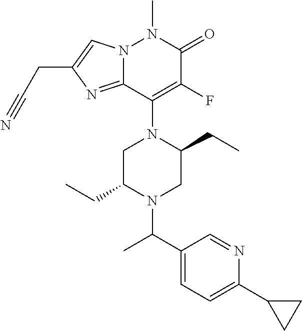 [SMILES: CC[C@H]1CN(C(C)c2ccc(C3CC3)nc2)[C@H](CC)CN1c1c(F)c(=O)n(C)n2cc(CC#N)nc12]

To a solution of 2-(8-((2S,5R)-4-(1-(6-cyclopropylpyridin-3-yl)ethyl)-2,5-diethylpiperazin-1-yl)-5-methyl-6-oxo-5,6-dihydroimidazo[1,2-b]pyridazin-2-yl)acetonitrile (142 mg, 0.3 mmol) in CH₃CN (10 mL) was added Selectfluor fluorinating reagent (117 mg, 0.33 mmol). The mixture was stirred at room temperature overnight. The reaction was poured into water, extracted with EtOAc (20 mL×2). The combined organic layers were washed with brine, dried over Na₂SO₄ and concentrated to dryness. The resulting residue was purified by flash column chromatography to give the titled Compound A73 (crude), which was further separated into Compound A73a (6 mg) and Compound A73b (7 mg) by Prep-HPLC(Method A).

Compound A73a (the earlier peak): ¹H NMR (400 MHz, CD₃OD) δ 8.30 (s, 1H), 7.78 (s, 1H), 7.72 (d, J=8.0 Hz, 1H), 7.20 (d, J=8.0 Hz, 1H), 4.68-4.59 (m, 1H), 3.94 (s, 2H), 3.89-3.80 (m, 2H), 3.78 (s, 3H), 3.68-3.59 (m, 1H), 3.13-3.03 (m, 1H), 2.98-2.88 (m, 1H), 2.37-2.28 (m, 1H), 2.15-1.87 (m, 3H), 1.70-1.46 (m, 2H), 1.35 (d, J=6.4 Hz, 3H), 0.94-0.89 (m, 4H), 0.87 (t, J=7.2 Hz, 3H), 0.59 (t, J=7.2 Hz, 3H) ppm. MS: M/e 492 (M+1)⁺

Compound A73b (the later peak): ¹H NMR (400 MHz, CD₃OD) δ 8.34 (s, 1H), 7.77 (s, 1H), 7.73 (d, J=8.0 Hz, 1H), 7.18 (d, J=8.0 Hz, 1H), 4.46-4.34 (m, 1H), 4.20-4.08 (m, 1H), 3.93 (s, 2H), 3.88-3.81 (m, 1H), 3.78 (s, 3H), 3.70-3.60 (m, 1H), 3.14-3.05 (m, 1H), 2.92-2.83 (m, 1H), 2.29-2.20 (m, 1H), 2.14-1.86 (m, 2H), 1.82-1.49 (m, 3H), 1.31 (d, J=6.4 Hz, 3H), 1.08-0.83 (m, 7H), 0.62 (t, J=7.2 Hz, 3H) ppm. MS: M/e 492 (M+1)⁺.

Compound A74: 2-(cyanomethyl)-8-((2S,5R)-4-(1-(6-cyclopropylpyridin-3-yl)ethyl)-5-ethyl-2-methylpiperazin-1-yl)-5-methyl-6-oxo-5,6-dihydroimidazo [1,2-b]pyridazine-7-carbonitrile

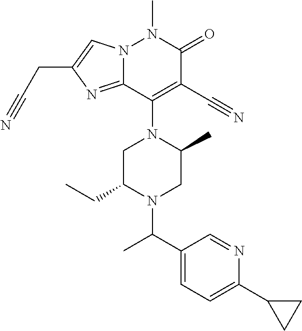 [SMILES: CC[C@@H]1CN(c2c(C#N)c(=O)n(C)n3cc(CC#N)nc23)[C@@H](C)CN1C(C)c1ccc(C2CC2)nc1]

### Step A: 2-(7-bromo-8-((2S,5R)-4-(1-(6-cyclopropylpyridin-3-yl)ethyl)-5-ethyl-2-methylpiperazin-1-yl)-5-methyl-6-oxo-5,6-dihydroimidazo[1,2-b]pyridazin-2-yl)acetonitrile

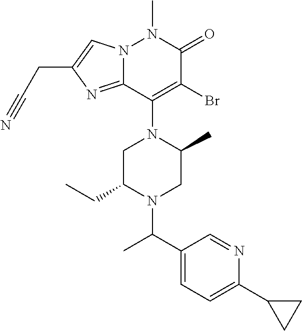 [SMILES: CC[C@@H]1CN(c2c(Br)c(=O)n(C)n3cc(CC#N)nc23)[C@@H](C)CN1C(C)c1ccc(C2CC2)nc1]

To a stirred solution of 2-(8-((2S,5R)-4-(1-(6-cyclopropylpyridin-3-yl)ethyl)-5-ethyl-2-methylpiperazin-1-yl)-5-methyl-6-oxo-5,6-dihydroimidazo[1,2-b]pyridazin-2-yl)acetonitrile (220 mg, 0.479 mmol) in CH₃CN (8 mL) was added NBS (94 mg, 0.53 mmol). After then, the mixture was stirred for 30 min. The reaction mixture was poured into H₂O (15 mL), then extracted with EtOAc (15 mL×3). The combined organic layers were washed with brine, dried over Na₂SO₄, concentrated and purified by flash column chromatography to give the titled compound (196 mg, 76%). MS: M/e 538/540 (M+1)⁺.

### Step B: 2-(cyanomethyl)-8-((2S,5R)-4-(1-(6-cyclopropylpyridin-3-yl)ethyl)-5-ethyl-2-methylpiperazin-1-yl)-5-methyl-6-oxo-5,6-dihydroimidazo[1,2-b]pyridazine-7-carbonitrile

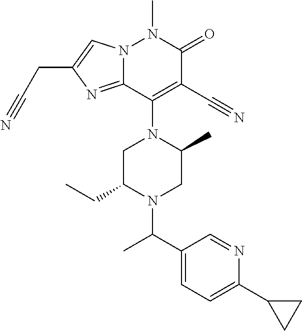 [SMILES: CC[C@@H]1CN(c2c(C#N)c(=O)n(C)n3cc(CC#N)nc23)[C@@H](C)CN1C(C)c1ccc(C2CC2)nc1]

To a stirred solution of 2-(7-bromo-8-((2S,5R)-4-(1-(6-cyclopropylpyridin-3-yl)ethyl)-5-ethyl-2-methylpiperazin-1-yl)-5-methyl-6-oxo-5,6-dihydroimidazo[1,2-b]pyridazin-2-yl)acetonitrile (196 mg, 0.364 mmol) in DMF (6 mL) was added Zn(CN)₂ (128 mg, 1.09 mmol), followed by Pd(PPh₃)₄(125 mg, 0.109 mmol). After the addition, the reaction mixture was stirred at 100° C. overnight under N₂. The reaction mixture was poured into H₂O (15 mL), then extracted with EtOAc (15 mL×3). The combined organic layers were washed with brine, dried over Na₂SO₄, concentrated to dryness. The resulting residue was purified by flash column chromatography to give the titled Compound A74 (crude), which was further separated into Compound A74a (30 mg) and Compound A74b (42 mg) by Prep-HPLC (Method A).

Compound A74a (the earlier peak): ¹H NMR (400 MHz, CD₃OD) δ 8.31 (s, 1H), 7.87 (s, 1H), 7.74 (d, J=8.0 Hz, 1H), 7.22 (d, J=8.0 Hz, 1H), 3.96 (s, 2H), 3.84-3.76 (m, 2H), 3.74 (s, 3H), 3.33-3.30 (m, 2H), 3.15-3.03 (m, 1H), 2.94-2.85 (m, 1H), 2.50-2.42 (m, 1H), 2.14-2.05 (m, 1H), 1.54 (d, J=6.0 Hz, 3H), 1.52-1.42 (m, 2H), 1.36 (d, J=6.4 Hz, 3H), 1.04-0.94 (m, 4H), 0.65 (t, J=7.2 Hz, 3H) ppm. MS: M/e 485 (M+1)⁺.

Compound A74b (the later peak): ¹H NMR (399 MHz, DMSO-d₆) δ 8.35 (s, 1H), 8.00 (s, 1H), 7.64 (d, J=8.0 Hz, 1H), 7.25 (d, J=8.0 Hz, 1H), 4.08 (s, 2H), 3.94-3.84 (m, 1H), 3.52-3.37 (m, 2H), 3.68-3.64 (m, 1H), 3.63 (s, 3H), 3.50-3.33 (m, 2H), 3.16-3.05 (m, 1H), 2.82-2.71 (m, 1H), 2.25-2.14 (m, 1H), 2.11-2.00 (m, 1H), 1.51-1.39 (m, 2H), 1.31-1.21 (m, 6H), 0.97-0.80 (m, 7H) ppm. MS: M/e 485 (M+1)⁺.

Compound A75: 2-(cyanomethyl)-8-((2S,5R)-4-(1-(6-cyclopropylpyridin-3-yl)ethyl)-2,5-diethylpiperazin-1-yl)-5-methyl-6-oxo-5,6-dihydroimidazo[1,2-b]pyridazine-7-carbonitrile

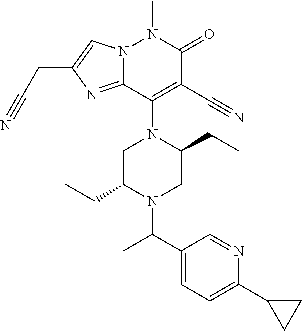 [SMILES: CC[C@H]1CN(C(C)c2ccc(C3CC3)nc2)[C@H](CC)CN1c1c(C#N)c(=O)n(C)n2cc(CC#N)nc12]

### Step A: 2-(7-bromo-8-((2S,5R)-4-(1-(6-cyclopropylpyridin-3-yl)ethyl)-2,5-diethylpiperazin-1-yl)-5-methyl-6-oxo-5,6-dihydroimidazo[1,2-b]pyridazin-2-yl)acetonitrile

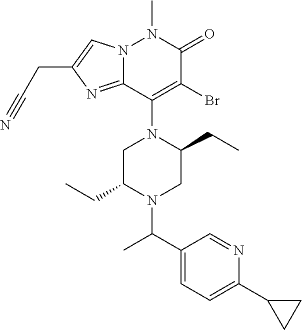 [SMILES: CC[C@H]1CN(C(C)c2ccc(C3CC3)nc2)[C@H](CC)CN1c1c(Br)c(=O)n(C)n2cc(CC#N)nc12]

To a stirred solution of 2-(8-((2S,5R)-4-(1-(6-cyclopropylpyridin-3-yl)ethyl)-2,5-diethylpiperazin-1-yl)-5-methyl-6-oxo-5,6-dihydroimidazo[1,2-b]pyridazin-2-yl)acetonitrile (200 mg, 0.42 mmol) in CH₃CN (8 mL) was added NBS (83 mg, 0.47 mmol). After then, the mixture was stirred for 30 min. The reaction mixture was poured into H₂O (10 mL), then extracted with EtOAc (15 mL×3). The combined organic layers were washed with brine, dried over Na₂SO₄, concentrated and purified by flash column chromatography to give the titled compound (194 mg, 76%). MS: M/e 552/554 (M+1)⁺.

### Step B: 2-(cyanomethyl)-8-((2S,5R)-4-(1-(6-cyclopropylpyridin-3-yl)ethyl)-2,5-diethylpiperazin-1-yl)-5-methyl-6-oxo-5,6-dihydroimidazo[1,2-b]pyridazine-7-carbonitrile

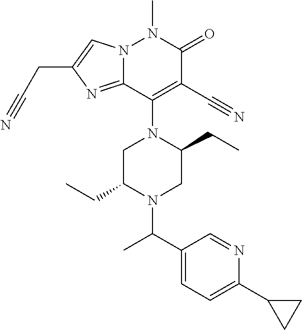 [SMILES: CC[C@H]1CN(C(C)c2ccc(C3CC3)nc2)[C@H](CC)CN1c1c(C#N)c(=O)n(C)n2cc(CC#N)nc12]

To a stirred solution of 2-(7-bromo-8-((2S,5R)-4-(1-(6-cyclopropylpyridin-3-yl)ethyl)-2,5-diethylpiperazin-1-yl)-5-methyl-6-oxo-5,6-dihydroimidazo[1,2-b]pyridazin-2-yl)acetonitrile (194 mg, 0.35 mmol) in DMF (6 mL) was added Zn(CN)₂ (123.3 mg, 1.05 mmol), followed by Pd(PPh₃)₄(121 mg, 0.105 mmol). After the addition, the reaction mixture was stirred at 100° C. overnight under N₂. The reaction mixture was poured into H₂O (15 mL), then extracted with EtOAc (15 mL×3). The combined organic layers were washed with brine, dried over Na₂SO₄, concentrated to dryness. The resulting residue was purified and separated into Compound A75a (7 mg) and Compound A75b (9 mg) by Prep-HPLC (Method A).

Compound A75a (the earlier peak): ¹H NMR (400 MHz, CD₃OD) δ 8.31 (s, 1H), 7.88 (s, 1H), 7.73 (d, J=8.0 Hz, 1H), 7.22 (d, J=8.0 Hz, 1H), 3.98 (s, 2H), 3.84-3.76 (m, 2H), 3.75 (s, 3H), 3.37-3.32 (m, 2H), 3.24-2.95 (m, 2H), 2.49-2.21 (m, 2H), 2.14-1.75 (m, 2H), 1.64-1.40 (m, 2H), 1.35 (d, J=6.0 Hz, 3H), 1.09-0.84 (m, 7H), 0.59 (t, J=7.2 Hz, 3H) ppm. MS: M/e 499 (M+1)⁺.

Compound A75b (the later peak): ¹H NMR (400 MHz, CD₃OD) δ 8.34 (s, 1H), 7.89 (s, 1H), 7.73 (d, J=8.0 Hz, 1H), 7.19 (d, J=8.0 Hz, 1H), 3.97 (s, 3H), 3.75 (s, 3H), 3.69-3.60 (m, 1H), 3.36-3.31 (m, 2H), 3.26-3.16 (m, 1H), 3.10-2.75 (m, 1H), 2.41-2.02 (m, 3H), 1.71-1.43 (m, 3H), 1.32 (d, J=6.0 Hz, 3H), 1.09-0.83 (m, 7H), 0.69 (t, J=7.2 Hz, 3H) ppm. MS: M/e 499 (M+1)⁺.

Compound A76: 2-(8-((2S,5R)-4-(1-(6-cyclobutylpyridin-3-yl)ethyl)-2,5-diethylpiperazin-1-yl)-5-methyl-6-oxo-5,6-dihydroimidazo[1,2-b]pyridazin-2-yl)acetonitrile

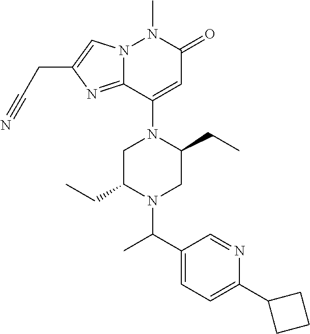 [SMILES: CC[C@H]1CN(C(C)c2ccc(C3CCC3)nc2)[C@H](CC)CN1c1cc(=O)n(C)n2cc(CC#N)nc12]

### Step A: 1-(5-bromopyridin-2-yl)cyclobutan-1-ol

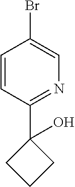 [SMILES: OC1(c2ccc(Br)cn2)CCC1]

To a solution of 5-bromo-2-iodopyridine (3.4 g, 12 mol) in THF (20 mL), cooled down to −40° C. and under N₂ atmosphere was added Isopropylmagnesium chloride (2M, 7.8 ml, 15.6 mol) by dropwise. The reaction mixture was stirred at −40° C. for 1 h. A solution of cyclobutanone (0.7 g, 10 mmol) in THF (5 ml) was slowly added to the mixture and the reaction was slowly warmed up to RT. The mixture was added H₂O (20 ml) and extracted with EA (20 mL×3), then concentrated by using a rotary evaporator to give a residue. The resulting residue was purified by flash column chromatography to give the titled compound (1 g, 44%). MS: M/e 228 (M+1)⁺.

### Step B: 1-(6-(1-hydroxycyclobutyl)pyridin-3-yl)ethan-1-one

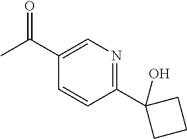

To a solution of 1-(5-bromopyridin-2-yl)cyclobutan-1-ol (400 mg, 1.76 mol) in toluene (10 mL) was added Pd(PPh₃)₂Cl₂ (140 mg, 0.2 mol) and tributyl(1-ethoxyvinyl)stannane (1.1 g, 3 mmol). The reaction mixture was protected by N₂ atmosphere and stirred at 90° C. for 16 hrs. The mixture was added 4N HCl in dionxane (0.5 ml) and stirred at RT for 15 mins. The mixture was added saturated NaHCO₃ aqueous solution (10 ml) and extracted with EA (20 mL×3). The organic phase was dried with Na₂SO₄ and concentrated by using a rotary evaporator to give a residue. The resulting residue was purified by flash column chromatography to give the titled compound (270 mg, 80%). MS: M/e 192 (M+1)⁺.

### Step C: 0-(1-(5-acetylpyridin-2-yl)cyclobutyl)S-methyl carbonodithioate

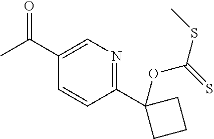 [SMILES: CSC(=S)OC1(c2ccc(C(C)=O)cn2)CCC1]

To a solution of 1-(6-(1-hydroxycyclobutyl)pyridin-3-yl)ethan-1-one (270 mg, 1.4 mol) and CS₂ (127 mg, 1.68 mmol) in THF (10 mL) was added NaH (60% in oil, 62 mg, 1.5 mol) at 0° C. After the addition, the reaction mixture was stirred at RT for 1 h and cooled down to 0° C. CH₃I (317 mg, 2.1 mmol) was added and the reaction mixture was continue stirred at RT for 1 h. The mixture was added H₂O (10 ml) and extracted with EA (20 mL×3). The organic phase was dried with Na₂SO₄ and concentrated by using a rotary evaporator to give a residue. The resulting residue was purified by flash column chromatography to give the titled compound (280 mg, 71.2%). MS: M/e 282 (M+1)⁺.

### Step D: 1-(6-cyclobutylpyridin-3-yl)ethan-1-one

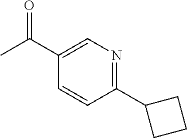

To a solution of O-(1-(5-acetylpyridin-2-yl)cyclobutyl)S-methyl carbonodithioate (70 mg, 0.25 mol) in toluene (10 mL) was added tributylstannane (144 mg, 0.5 mol) and AIBN (8.2 mg, 0.05 mmol). The reaction mixture was protected by N₂ atmoshpere and stirred at 100° C. for 1 h. The mixture was added H₂O (10 ml) and extracted with EA (20 mL×3). The organic phase was dried with Na₂SO₄ and concentrated by using a rotary evaporator to give a residue. The resulting residue was purified by flash column chromatography to give the titled compound (30 mg, 69.7%). MS: M/e 175 (M+1)⁺.

### Step E: 1-(6-cyclobutylpyridin-3-yl)ethan-1-ol

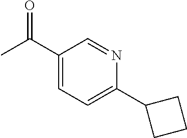

To a solution of 1-(6-cyclobutylpyridin-3-yl)ethan-1-one (30 mg, 0.17 mol) in MeOH was added NaBH₄ (19 mg, 0.5 mmol). The reaction mixture was stirred at RT for 15 mins. The mixture was added H₂O (20 ml) and extracted with DCM (20 mL×3). The organic phase was dried with Na₂SO₄ and concentrated by using a rotary evaporator to give a residue. The resulting residue was purified by flash column chromatography to give the titled compound (30 mg, 100%). MS: M/e 177 (M+1)⁺.

### Step F: 2-(8-((2S,5R)-4-(1-(6-cyclobutylpyridin-3-yl)ethyl)-2,5-diethylpiperazin-1-yl)-5-methyl-6-oxo-5,6-dihydroimidazo[1,2-b]pyridazin-2-yl)acetonitrile

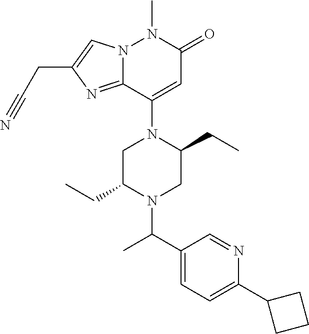 [SMILES: CC[C@H]1CN(C(C)c2ccc(C3CCC3)nc2)[C@H](CC)CN1c1cc(=O)n(C)n2cc(CC#N)nc12]

To a solution of 2-(8-((2S,5R)-2,5-diethylpiperazin-1-yl)-5-methyl-6-oxo-5,6-dihydroimidazo[1,2-b]pyridazin-2-yl)acetonitrile (30 mg, 0.1 mmol), 1-(6-cyclobutylpyridin-3-yl)ethan-1-ol (30 mg, 0.18 mmol) and (cyanomethyl)trimethylphosphonium iodide (120 mg, 0.5 mmol) in CH₃CN (10 mL) was added DIPEA (258 mg, 2 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified and separated into Compound A76a (1.5 mg) and Compound A76b (1.8 mg) by Prep-HPLC(method A).

Compound A76a (the earlier peak): ¹H NMR (400 MHz, CD₃OD) δ 8.41 (s, 1H), 7.80 (d, J=11.8 Hz, 2H), 7.35 (d, J=7.9 Hz, 1H), 5.61 (s, 1H), 3.93 (s, 2H), 3.83 (d, J=6.0 Hz, 1H), 3.71 (m, 4H), 3.27 (m, 2H), 3.00 (d, J=12.1 Hz, 1H), 2.90 (d, J=12.0 Hz, 1H), 2.33 (m, 5H), 2.11 (m, 3H), 1.89 (m, 2H), 1.54 (m, 2H), 1.37 (d, J=6.0 Hz, 3H), 0.94 (d, J=6.8 Hz, 3H), 0.73 (t, J=6.8 Hz, 3H) ppm. MS: M/e 488 (M+1)⁺.

Compound A76b (the later peak): ¹H NMR (400 MHz, CD₃OD) δ 8.45 (s, 1H), 7.81 (d, J=13.3 Hz, 2H), 7.32 (d, J=7.9 Hz, 1H), 5.60 (s, 1H), 3.94 (s, 2H), 3.70 (m, 5H), 3.50 (d, J=12.7 Hz, 1H), 3.34 (s, 1H), 3.16 (d, J=9.9 Hz, 1H), 2.69 (d, J=11.4 Hz, 1H), 2.33 (m, 5H), 2.07 (m, 2H), 1.92 (s, 2H), 1.62 (d, J=46.9 Hz, 3H), 1.30 (s, 3H), 1.03 (s, 3H), 0.62 (s, 3H) ppm. MS: M/e 488 (M+1)⁺.

Compound A77: 2-(8-((2S,5R)-2,5-diethyl-4-(1-(4-(trifluoromethyl)phenyl)propyl)piperazin-1-yl)-5-methyl-6-oxo-5,6-dihydroimidazo[1,2-b]pyridazin-2-yl)acetonitrile

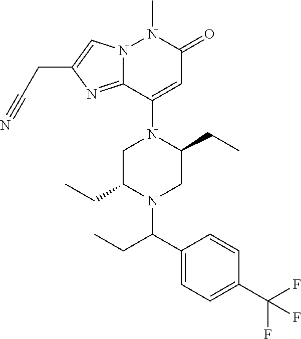 [SMILES: CCC(c1ccc(C(F)(F)F)cc1)N1C[C@H](CC)N(c2cc(=O)n(C)n3cc(CC#N)nc23)C[C@H]1CC]

A mixture of 1-(4-(trifluoromethyl)phenyl)propan-1-ol (122 mg, 0.6 mmol), 2-(8-((2S,5R)-2,5-diethylpiperazin-1-yl)-5-methyl-6-oxo-5,6-dihydroimidazo[1,2-b]pyridazin-2-yl)acetonitrile (100 mg, 0.3 mmol), (cyanomethyl) trimethylphosphonium iodide (290 mg, 1.2 mmol) and DIPEA (155 mg, 1.2 mmol) in CH₃CN (3 mL) was stirred at 100° C. overnight in a sealed tube. The reaction mixture was diluted with EtOAc (20 mL), washed with water (10 mL×2), dried over Na₂SO₄ and concentrated to dryness. The resulting residue was purified and separated into Compound A77a (14 mg) and Compound A77b (16 mg) by Prep-HPLC (Method A).

Compound A77a (the earlier peak): ¹H NMR (400 MHz, DMSO-d₆) δ 7.84 (s, 1H), 7.71 (d, J=7.9 Hz, 2H), 7.54 (d, J=7.7 Hz, 2H), 5.48 (s, 1H), 4.00 (s, 2H), 3.59 (s, 4H), 3.32-3.27 (m, 2H), 3.11 (d, J=12.5 Hz, 1H), 2.82 (s, 2H), 2.25 (d, J=7.4 Hz, 1H), 1.91 (s, 2H), 1.74-1.50 (m, 2H), 1.40 (s, 2H), 0.86 (s, 3H), 0.61 (s, 6H) ppm. MS: M/e 515 (M+1)⁺.

Compound A77b (the later peak): ¹H NMR (400 MHz, DMSO-d₆) δ 7.84 (s, 1H), 7.69 (d, J=7.4 Hz, 2H), 7.55 (d, J=7.3 Hz, 2H), 5.48 (s, 1H), 4.01 (s, 2H), 3.59 (s, 3H), 3.50-3.46 (m, 2H), 3.34 (s, 1H), 3.04 (s, 1H), 2.51-2.49 (m, 2H), 2.12 (d, J=11.8 Hz, 1H), 1.83 (s, 2H), 1.65-1.33 (m, 4H), 0.95 (s, 3H), 0.66-0.40 (m, 6H) ppm. MS: M/e 515 (M+1)⁺.

Compound A78:-2-(8-((2S,5R)-4-(1-(5-(difluoromethyl)pyridin-2-yl)ethyl)-2,5-diethylpiperazin-1-yl)-5-methyl-6-oxo-5,6-dihydroimidazo[1,2-b]pyridazin-2-yl)acetonitrile

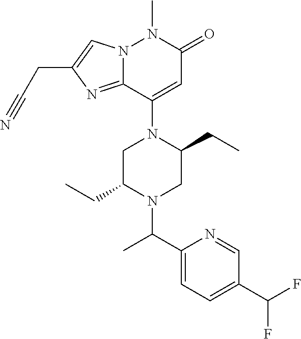 [SMILES: CC[C@H]1CN(C(C)c2ccc(C(F)F)cn2)[C@H](CC)CN1c1cc(=O)n(C)n2cc(CC#N)nc12]

To a solution of 2-(8-((2S,5R)-2,5-diethylpiperazin-1-yl)-5-methyl-6-oxo-5,6-dihydroimidazo[1,2-b]pyridazin-2-yl)acetonitrile (100 mg, 0.30 mmol) in CH₃CN (3 mL) and was added 1-(5-(difluoromethyl)pyridin-2-yl)ethan-1-ol (53 mg, 0.30 mmol), (cyanomethyl)trimethylphosphonium iodide (223 mg, 0.91 mmol) and DIPEA (197 mg, 1.52 mmol). The resulting mixture was stirred at 105° C. overnight. The reaction solvent was removed under reduce pressure to afford crude product. The resulting residue was purified and separated into Compound A78a (18 mg) and Compound A78b (17 mg) by Prep-HPLC(Method B).

Compound A78a (the earlier peak): ¹H NMR (400 MHz, CD₃OD) δ 8.63 (s, 1H), 8.03 (d, J=7.9 Hz, 1H), 7.79 (s, 1H), 7.71 (d, J=8.3 Hz, 1H), 6.73 (t, J=55.5 Hz, 1H), 5.62 (s, 1H), 3.94 (s, 1H), 3.93 (s, 2H), 3.74 (s, 3H), 3.31-3.29 (m, 3H), 3.01 (d, J=12.7 Hz, 1H), 2.93 (d, J=12.1 Hz, 1H), 2.37 (s, 1H), 2.15-2.04 (m, 1H), 1.89-1.80 (m, 1H), 1.62-1.51 (m, 2H), 1.40 (d, J=6.3 Hz, 3H), 1.01-0.88 (m, 3H), 0.81-0.67 (m, 3H) ppm. MS: M/e 484(M+1)⁺.

Compound A78b (the later peak): ¹H NMR (400 MHz, CD₃OD) δ 8.67 (s, 1H), 8.05 (d, J=8.0 Hz, 1H), 7.79 (s, 1H), 7.68 (d, J=7.9 Hz, 1H), 6.73 (t, J=54.8 Hz, 1H), 5.61 (s, 1H), 3.94 (s, 2H), 3.78 (d, J=5.8 Hz, 1H), 3.74 (s, 3H), 3.52 (d, J=13.3 Hz, 1H), 3.30-3.18 (m, 3H), 2.74 (d, J=11.9 Hz, 1H), 2.28 (d, J=12.4 Hz, 1H), 1.96-1.85 (m, 1H), 1.76-1.64 (m, 2H), 1.61-1.53 (m, 1H), 1.37 (d, J=6.1 Hz, 3H), 1.09-1.00 (m, 3H), 0.68-0.57 (m, 3H) ppm. MS: M/e 484(M+1)⁺.

Compound A79: 2-(8-((2S,5R)-2,5-diethyl-4-(1-(5-(trifluoromethyl)pyridin-2-yl)ethyl)piperazin-1-yl)-5-methyl-6-oxo-5,6-dihydroimidazo[1,2-b]pyridazin-2-yl)acetonitrile

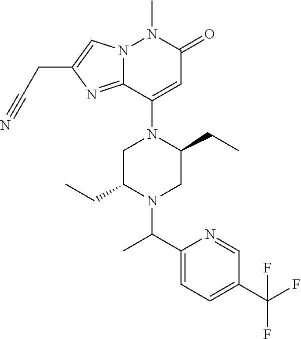 [SMILES: CC[C@H]1CN(C(C)c2ccc(C(F)(F)F)cn2)[C@H](CC)CN1c1cc(=O)n(C)n2cc(CC#N)nc12]

To a solution of 2-(8-((2S,5R)-2,5-diethylpiperazin-1-yl)-5-methyl-6-oxo-5,6-dihydroimidazo[1,2-b]pyridazin-2-yl)acetonitrile (100 mg, 0.30 mmol) in CH₃CN (3 mL) and was added 1-(5-(trifluoromethyl)pyridin-2-yl)ethan-1-ol (53 mg, 0.30 mmol), (cyanomethyl)trimethylphosphonium iodide (223 mg, 0.91 mmol) and DIPEA (197 mg, 1.52 mmol). The resulting mixture was stirred at 105° C. overnight. The reaction solvent was removed under reduce pressure to afford crude product. The resulting residue was purified and separated into Compound A79a (20 mg) and Compound A79b (20 mg) by Prep-HPLC(Method B).

Compound A79a (the earlier peak): ¹H NMR (400 MHz, CD₃OD) δ 8.72 (s, 1H), 8.09 (d, J=8.8 Hz, 1H), 7.82 (d, J=8.1 Hz, 1H), 7.79 (s, 1H), 5.62 (s, 1H), 4.02-3.96 (m, 1H), 3.93 (s, 2H), 3.74 (s, 3H), 3.35-3.30 (m, 2H), 3.15-2.91 (m, 2H), 2.39-2.34 (m, 1H), 2.13-2.05 (m, 1H), 1.89-1.82 (m, 1H), 1.62-1.54 (m, 2H), 1.41 (d, J=6.3 Hz, 3H), 1.32-1.28 (m, 1H), 0.98-0.91 (m, 3H), 0.79-0.71 (m, 3H) ppm. MS: M/e 502(M+1)⁺.

Compound A79b (the later peak): ¹H NMR (400 MHz, CD₃OD) δ 8.75 (s, 1H), 8.10 (d, J=7.5 Hz, 1H), 7.81 (s, 1H), 7.79 (s, 1H), 5.61 (s, 1H), 3.94 (s, 2H), 3.82 (d, J=6.1 Hz, 1H), 3.74 (s, 3H), 3.52 (d, J=13.5 Hz, 1H), 3.30-3.18 (m, 2H), 2.75 (d, J=12.8 Hz, 1H), 2.26 (d, J=12.4 Hz, 1H), 1.95-1.86 (m, 1H), 1.76-1.64 (m, 2H), 1.60-1.53 (m, 1H), 1.38 (d, J=6.2 Hz, 3H), 1.31-1.28 (m, 1H), 1.04 (t, J=6.9 Hz, 3H), 0.63 (t, J=6.9 Hz, 3H) ppm. MS: M/e 502(M+1)⁺.

Compound A80: 2-(8-((2S,5R)-5-ethyl-2-methyl-4-(1-(thiazolo[5,4-b]pyridin-5-yl)ethyl)piperazin-1-yl)-5-methyl-6-oxo-5,6-dihydroimidazo[1,2-b]pyridazin-2-yl)acetonitrile

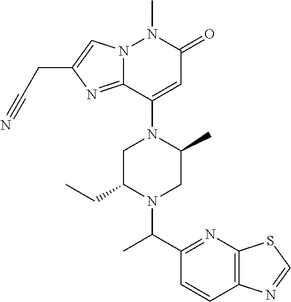 [SMILES: CC[C@@H]1CN(c2cc(=O)n(C)n3cc(CC#N)nc23)[C@@H](C)CN1C(C)c1ccc2ncsc2n1]

To a solution of 2-(8-((2S,5R)-5-ethyl-2-methylpiperazin-1-yl)-5-methyl-6-oxo-5,6-dihydroimidazo[1,2-b]pyridazin-2-yl)acetonitrile (50 mg, 0.15 mmol) in CH₃CN (3 mL) and was added 1-(thiazolo[5,4-b]pyridin-5-yl)ethan-1-ol (57 mg, 0.32 mmol), (cyanomethyl)trimethylphosphonium iodide (116 mg, 0.47 mmol) and DIPEA (205 mg, 1.5 mmol). The resulting mixture was stirred at 105° C. overnight. The reaction solvent was removed under reduce pressure. The resulting residue was purified by flash column chromatography to give the titled Compound A80(crude), which was further separated into Compound A80a (15 mg) and Compound A80b (8 mg) by Prep-HPLC (Method B).

Compound A80a (the earlier peak):¹H NMR (400 MHz, CD₃OD) δ 9.35 (s, 1H), 8.43 (d, J=8.5 Hz, 1H), 7.80 (d, J=9.1 Hz, 2H), 5.64 (s, 1H), 5.10 (s, 1H), 4.78 (s, 1H), 4.18-4.07 (m, 1H), 3.91 (s, 2H), 3.74 (s, 3H), 3.36 (d, J=14.1 Hz, 1H), 3.07 (d, J=12.0 Hz, 1H), 2.87 (d, J=11.9 Hz, 1H), 2.42 (s, 1H), 1.69-1.55 (m, 2H), 1.45 (d, J=6.6 Hz, 3H), 1.40 (d, J=6.4 Hz, 3H), 0.77 (t, J=7.3 Hz, 3H) ppm. MS: M/e 477 (M+1)⁺.

Compound A80b (the later peak):¹H NMR (400 MHz, CD₃OD) δ 9.34 (s, 1H), 8.41 (d, J=8.5 Hz, 1H), 7.88 (d, J=8.6 Hz, 1H), 7.79 (s, 1H), 5.63 (s, 1H), 5.41 (s, 1H), 4.53 (s, 1H), 3.94 (s, 3H), 3.74 (s, 3H), 3.58 (d, J=13.5 Hz, 1H), 3.19 (d, J=10.6 Hz, 1H), 2.87 (dd, J=12.0, 3.9 Hz, 1H), 2.19 (d, J=12.4 Hz, 1H), 1.81-1.58 (m, 2H), 1.43 (d, J=6.6 Hz, 3H), 1.25 (d, J=6.5 Hz, 3H), 1.08 (t, J=7.3 Hz, 3H) ppm. MS: M/e 477 (M+1)⁺.

Compound A81: 2-(8-((2S,5R)-2,5-diethyl-4-(1-(thiazolo[5,4-b]pyridin-5-yl)ethyl)piperazin-1-yl)-7-fluoro-5-methyl-6-oxo-5,6-dihydroimidazo[1,2-b]pyridazin-2-yl)acetonitrile

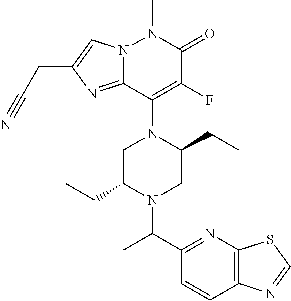 [SMILES: CC[C@H]1CN(C(C)c2ccc3ncsc3n2)[C@H](CC)CN1c1c(F)c(=O)n(C)n2cc(CC#N)nc12]

To a solution of 2-(8-((2S,5R)-2,5-diethyl-4-(1-(thiazolo[5,4-b]pyridin-5-yl)ethyl)piperazin-1-yl)-5-methyl-6-oxo-5,6-dihydroimidazo[1,2-b]pyridazin-2-yl)acetonitrile (100 mg, 0.20 mmol) in CH₃CN (4 mL) was added 1-chloromethyl-4-fluoro-1,4-diazoniabicyclo[2.2.2]octane bis(tetrafluoroborate) (108 mg, 0.31 mmol). The resulting mixture was stirred at room temperature for 5 mins. The reaction solvent was collected by filtration and removed under reduce pressure. The resulting residue was purified by flash column chromatography to give the titled Compound A81(crude), which was further separated into Compound A81a (17 mg) and Compound A81b (14 mg) by Prep-HPLC (Method A).

Compound A81a (the earlier peak):1H NMR (400 MHz, CD₃OD) δ 9.34 (s, 1H), 8.42 (d, J=8.4 Hz, 1H), 7.86 (d, J=8.6 Hz, 1H), 7.78 (s, 1H), 4.42 (s, 1H), 4.15 (d, J=12.5 Hz, 1H), 3.91 (d, J=15.0 Hz, 4H), 3.78 (s, 3H), 3.14 (d, J=10.0 Hz, 1H), 3.00 (d, J=12.0 Hz, 1H), 2.19 (d, J=11.6 Hz, 1H), 2.02-1.50 (m, 4H), 1.40 (d, J=6.3 Hz, 3H), 0.90 (t, J=7.0 Hz, 3H), 0.62 (t, J=7.1 Hz, 3H) ppm. MS: M/e 509 (M+1)⁺.

Compound A81b (the later peak):¹H NMR (400 MHz, CD₃OD) δ 9.34 (s, 1H), 8.43 (d, J=8.6 Hz, 1H), 7.82 (d, J=7.8 Hz, 1H), 7.78 (s, 1H), 4.65 (s, 1H), 4.13 (d, J=5.9 Hz, 1H), 3.94 (s, 2H), 3.84 (d, J=13.0 Hz, 1H), 3.78 (s, 3H), 3.71 (d, J=12.7 Hz, 1H), 3.17 (d, J=11.6 Hz, 1H), 2.96 (d, J=11.9 Hz, 1H), 2.32 (s, 1H), 2.20-1.59 (m, 4H), 1.44 (d, J=6.4 Hz, 3H), 0.88 (t, J=7.2 Hz, 3H), 0.58 (t, J=7.0 Hz, 3H) ppm. MS: M/e 509(M+1)⁺.

Compound A82: 2-(8-((2S,5R)-2,5-diethyl-4-(1-(2-methylthiazolo[5,4-b]pyridin-5-yl)ethyl)piperazin-1-yl)-7-fluoro-5-methyl-6-oxo-5,6-dihydroimidazo[1,2-b]pyridazin-2-yl)acetonitrile

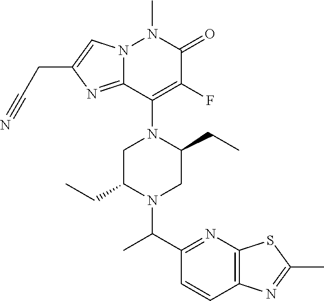 [SMILES: CC[C@H]1CN(C(C)c2ccc3nc(C)sc3n2)[C@H](CC)CN1c1c(F)c(=O)n(C)n2cc(CC#N)nc12]

To a solution of 2-(8-((2S,5R)-2,5-diethyl-4-(1-(2-methylthiazolo[5,4-b]pyridin-5-yl)ethyl)piperazin-1-yl)-5-methyl-6-oxo-5,6-dihydroimidazo[1,2-b]pyridazin-2-yl)acetonitrile (80 mg, 0.16 mmol) in MeCN (2 mL) was added 1-ChloroMethyl-4-fluoro-1,4-diazoniabicyclo[2.2.2]octane bis(tetrafluoroborate) (80 mg, 0.22 mmol) at RT and the mixture was stirred at RT for 30 minutes. The mixture was diluted with EA (20 mL), washed with brine (10 mL×3), dried over Na₂SO₄, filtered and concentrated to dryness. The resulted residue was purified by column chromatography to give the titled Compound A82, which was further separated into CompoundA82a (14 mg) and CompoundA82b (14 mg) by Prep-Chiral-HPLC The chiral separation conditions are shown below.

| Column | IE |
|---|---|
| Column Size | 20.0 mm × 250 mm, 5 um |
| Mobile Phase A | Hex |
| Mobile Phase B | EtOH (0.2% 2M NH₃ MeOH) |
| Flow Rate | 18 mL/min |
| Gradient | Mobile Phase A:Mobile Phase B = 40:60(v/v) |
| Wave Length | UV 220 nm and 300 nm |
| Temperature | RT |
| Prep-HPLC Equipment | Prep-HPLC-GilsonB |

Compound A82a (the earlier peak): ¹H NMR (400 MHz, CD₃OD) δ 8.24 (d, J=8.4 Hz, 1H), 7.78 (s, 1H), 7.74 (d, J=8.4 Hz, 1H), 4.64 (s, 1H), 4.07 (q, J=6.4 Hz, 1H), 3.94 (s, 2H), 3.89-3.75 (m, 4H), 3.70 (d, J=13.2 Hz, 1H), 3.15 (dd, J=11.6, 3.6 Hz, 1H), 2.95 (d, J=11.6 Hz, 1H), 2.86 (s, 3H), 2.32 (d, J=8.4 Hz, 1H), 2.20-2.05 (m, 1H), 2.03-1.89 (m, 1H), 1.73-1.57 (m, 2H), 1.42 (d, J=6.4 Hz, 3H), 0.88 (t, J=7.6 Hz, 3H), 0.58 (t, J=7.6 Hz, 3H). MS: M/e 523 (M+1)⁺.

Compound A82b (the later peak): ¹H NMR (400 MHz, CD₃OD) δ 8.22 (d, J=8.8 Hz, 1H), 7.83-7.74 (m, 2H), 4.41 (s, 1H), 4.14 (d, J=12.8 Hz, 1H), 3.96-3.84 (m, 4H), 3.78 (s, 3H), 3.12 (d, J=9.6 Hz, 1H), 2.98 (dd, J=12.0, 3.6 Hz, 1H), 2.86 (s, 3H), 2.18 (d, J=11.2 Hz, 1H), 2.05-1.91 (m, 1H), 1.86-1.72 (m, 2H), 1.64-1.49 (m, 1H), 1.38 (d, J=6.8 Hz, 3H), 0.90 (t, J=7.6 Hz, 3H), 0.62 (t, J=7.6 Hz, 3H). MS: M/e 523 (M+1)⁺.

Compound A83: 2-(7-bromo-8-((2S,5R)-2,5-diethyl-4-(1-(2-methylthiazolo[5,4-b]pyridin-5-yl)ethyl)piperazin-1-yl)-5-methyl-6-oxo-5,6-dihydroimidazo[1,2-b]pyridazin-2-yl)acetonitrile

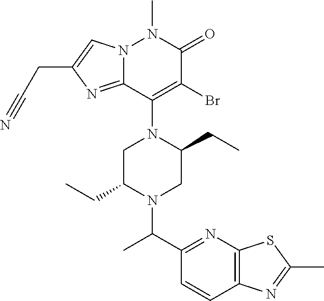 [SMILES: CC[C@H]1CN(C(C)c2ccc3nc(C)sc3n2)[C@H](CC)CN1c1c(Br)c(=O)n(C)n2cc(CC#N)nc12]

To a solution of 2-(8-((2S,5R)-2,5-diethyl-4-(1-(2-methylthiazolo[5,4-b]pyridin-5-yl)ethyl)piperazin-1-yl)-5-methyl-6-oxo-5,6-dihydroimidazo[1,2-b]pyridazin-2-yl)acetonitrile (140 mg, 0.28 mmol) in CH₃CN (2 mL) was added NBS (55 mg, 0.31 mmol) at room temperature and the mixture was stirred at room temperature for 30 minutes. The mixture was diluted with EtOAc (20 mL), washed with brine (10 mL×3), dried over Na₂SO₄, filtered and concentrated to dryness. The resulted residue was purified by column chromatography to give the titled compound (112 mg, 79%). ¹H NMR (400 MHz, CD₃OD) δ 8.29-8.17 (m, 1H), 7.82 (s, 1H), 7.81-7.70 (m, 1H), 4.72-4.22 (m, 1H), 4.20-4.02 (m, 1H), 4.00-3.93 (m, 2H), 3.87-3.81 (m, 3H), 3.81-3.64 (m, 1H), 3.52-3.34 (m, 1H), 3.27-3.06 (m, 1H), 2.93-2.87 (m, 1H), 2.86-2.67 (m, 3H), 2.41-2.16 (m, 1H), 2.16-1.95 (m, 1H), 1.89-1.48 (m, 3H), 1.47-1.34 (m, 3H), 0.91-0.73 (m, 3H), 0.69-0.41 (m, 3H) ppm. MS: M/e 583, 585 (M+1)⁺.

CompoundA84: 2-(8-((2S,5R)-5-ethyl-2-methyl-4-(1-(2-methylthiazolo[5,4-b]pyridin-5-yl)ethyl)piperazin-1-yl)-5-methyl-6-oxo-5,6-dihydroimidazo[1,2-b]pyridazin-2-yl)acetonitrile

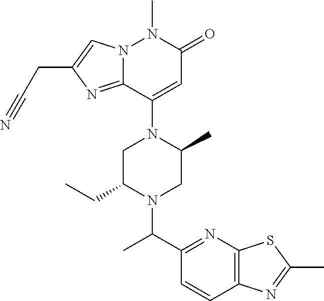 [SMILES: CC[C@@H]1CN(c2cc(=O)n(C)n3cc(CC#N)nc23)[C@@H](C)CN1C(C)c1ccc2nc(C)sc2n1]

To a solution of 2-(8-((2S,5R)-5-ethyl-2-methylpiperazin-1-yl)-5-methyl-6-oxo-5,6-dihydroimidazo[1,2-b]pyridazin-2-yl)acetonitrile (189 mg, 0.6 mmol), 1-(2-methylthiazolo[5,4-b]pyridin-5-yl)ethan-1-ol (175 mg, 0.9 mmol) and (cyanomethyl)trimethylphosphonium iodide (440 mg, 1.8 mmol) in CH₃CN (2 mL) was added DIPEA (400 mg, 3.1 mmol). The mixture was stirred at 100° C. in a sealed tube for 16 hours. The mixture was cooled and diluted with EtOAc (20 mL), washed with brine (10 mL×3), dried over Na₂SO₄, filtered and concentrated under reduced pressure. The resulting residue was purified by flash column chromatography to give the titled Compound A84 (280 mg). Then Compound A84 (50 mg) was further separated into Compound A84a (11 mg) and Compound A84b (11 mg) by Prep-HPLC (Method B).

Compound A84a (the earlier peak): ¹H NMR (400 MHz, CD₃OD) δ 8.23 (d, J=8.4 Hz, 1H), 7.78 (s, 1H), 7.71 (d, J=8.4 Hz, 1H), 5.63 (s, 1H), 5.38-4.93 (m, 1H), 4.85-4.59 (m, 1H), 4.07 (q, J=6.4 Hz, 1H), 3.91 (s, 2H), 3.73 (s, 3H), 3.38-3.32 (m, 1H), 3.05 (dd, J=12.0, 4.0 Hz, 1H), 2.92-2.79 (m, 4H), 2.49-2.38 (m, 1H), 1.67-1.50 (m, 2H), 1.43 (d, J=6.4 Hz, 3H), 1.39 (d, J=6.4 Hz, 3H), 0.76 (t, J=7.2 Hz, 3H) ppm. MS: M/e 491 (M+1)⁺.

Compound A84b (the later peak): ¹H NMR (400 MHz, CD₃OD) δ 8.21 (d, J=8.8 Hz, 1H), 7.83-7.75 (m, 2H), 5.62 (s, 1H), 5.55-5.45 (m, 1H), 4.72-4.25 (m, 1H), 4.02-3.83 (m, 3H), 3.74 (s, 3H), 3.61-3.52 (m, 1H), 3.21-3.13 (m, 1H), 2.92-2.79 (m, 4H), 2.26-2.12 (m, 1H), 1.80-1.64 (m, 1H), 1.61-1.49 (m, 1H), 1.41 (d, J=6.4 Hz, 3H), 1.24 (d, J=6.4 Hz, 3H), 1.07 (t, J=7.2 Hz, 3H) ppm. MS: M/e 491 (M+1)⁺.

Compound A85: 2-(8-((2S,5R)-5-ethyl-2-methyl-4-(1-(2-methylthiazolo[5,4-b]pyridin-5-yl)ethyl)piperazin-1-yl)-7-fluoro-5-methyl-6-oxo-5,6-dihydroimidazo[12-b]pyridazin-2-yl)acetonitrile

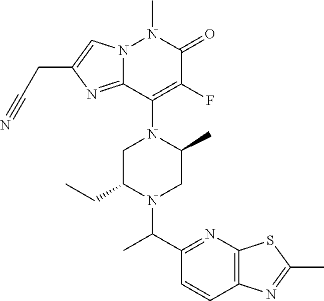 [SMILES: CC[C@@H]1CN(c2c(F)c(=O)n(C)n3cc(CC#N)nc23)[C@@H](C)CN1C(C)c1ccc2nc(C)sc2n1]

To a solution of 2-(8-((2S,5R)-5-ethyl-2-methyl-4-(1-(2-methylthiazolo[5,4-b]pyridin-5-yl)ethyl)piperazin-1-yl)-5-methyl-6-oxo-5,6-dihydroimidazo[1,2-b]pyridazin-2-yl)acetonitrile (80 mg, 0.16 mmol) in CH₃CN (2 mL) was added 1-ChloroMethyl-4-fluoro-1,4-diazoniabicyclo[2.2.2]octane bis(tetrafluoroborate) (75 mg, 0.21 mmol) at RT and the mixture was stirred at RT for 30 minutes. The mixture was diluted with EtOAc (20 mL), washed with brine (10 mL×3), dried over Na₂SO₄, filtered and concentrated to dryness. The resulted residue was purified by column chromatography to give the titled compound (112 mg, 79%). ¹H NMR (400 MHz, CD₃OD) δ 8.30-8.17 (m, 1H), 7.84-7.67 (m, 2H), 4.78-4.57 (m, 1H), 4.28-4.02 (m, 1H), 4.02-3.89 (m, 3H), 3.84-3.70 (m, 4H), 3.22-3.04 (m, 1H), 3.04-2.72 (m, 4H), 2.48-2.10 (m, 1H), 1.83-1.55 (m, 2H), 1.49-1.37 (m, 4H), 1.29-1.23 (m, 2H), 0.97-0.64 (m, 3H) ppm. MS: M/e 509 (M+1)⁺.

Compound A86: 2-(8-((2S,5R)-5-ethyl-2-methyl-4-(1-(thieno[2,3-b]pyridin-6-yl)ethyl)piperazin-1-yl)-5-methyl-6-oxo-5,6-dihydroimidazo[1,2-b]pyridazin-2-yl)acetonitrile

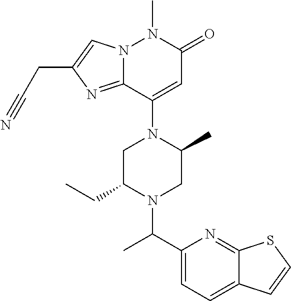 [SMILES: CC[C@@H]1CN(c2cc(=O)n(C)n3cc(CC#N)nc23)[C@@H](C)CN1C(C)c1ccc2ccsc2n1]

### Step A: thieno[2,3-b]pyridine 7-oxide

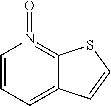

A solution of thieno[2,3-b]pyridine (260 mg, 1.74 mmol) and mCPBA (604 mg, 3.49 mmol) in DCM (10 ml) was stirred at RT overnight. The reaction was washed with brine (10 ml×2), dried over Na₂SO₄ and concentrated to dryness. The resulting residue was purified by flash column chromatography with 0-5% MeOH in DCM to give the titled compound (200 mg, 69%). MS: M/e 152 (M+1)⁺.

### Step B: thieno[2,3-b]pyridine-6-carbonitrile

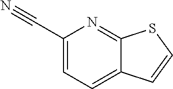 [SMILES: N#Cc1ccc2ccsc2n1]

A solution of thieno[2,3-b]pyridine 7-oxide (220 mg, 1.33 mmol), TMSCN (396 mg, 4 mmol), dimethylcarbamic chloride (432 mg, 4 mmol) and TEA (404 mg, 4 mmol) in MeCN (4 ml) was stirred at 70° C. for 3 hrs. The solution was diluted with EtOAc (15 ml), washed with brine (10 ml×2), dried over Na₂SO₄ and concentrated to dryness. The resulting residue was purified by flash column chromatography with 0-20% EA in PE to give the titled compound (175 mg, 75%). MS: M/e 161 (M+1)⁺.

### Step C: 1-(thieno[2,3-b]pyridin-6-yl)ethan-1-one

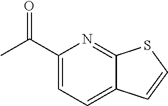

A solution of thieno[2,3-b]pyridine-6-carbonitrile (200 mg, 1.15 mmol), MeMgBr (1M, 1.73 ml, 1.73 mmol) and CuBr (16.5 mg, 0.115 mmol) in THF (4 ml) under N₂ was stirred at 60° C. overnight. The solution was diluted with EtOAc (10 ml), washed with brine (10 ml×2), dried over Na₂SO₄ and concentrated to dryness. The resulting residue was purified by flash column chromatography with 0-15% EA in PE to give the titled compound (100 mg, 46%). MS: M/e 178 (M+1)⁺.

### Step D: 1-(thieno[2,3-b]pyridin-6-yl)ethan-1-ol

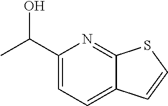

A solution of 1-(thieno[2,3-b]pyridin-6-yl)ethan-1-one (100 mg, 0.52 mmol) and NaBH₄ (20 mg, 0.53 mmol) in MeOH (2 ml) was stirred at 0° C. for 15 min. The solution was diluted with EtOAc (10 ml), washed with brine (10 ml×2), dried over Na₂SO₄ and concentrated to dryness to give the titled compound (100 mg, 99%), which was used directly for the next step without further purification. MS: M/e 180 (M+1)⁺.

### Step E: 2-(8-((2S,5R)-5-ethyl-2-methyl-4-(1-(thieno[2,3-b]pyridin-6-yl)ethyl)piperazin-1-yl)-5-methyl-6-oxo-5,6-dihydroimidazo[1,2-b]pyridazin-2-yl)acetonitrile

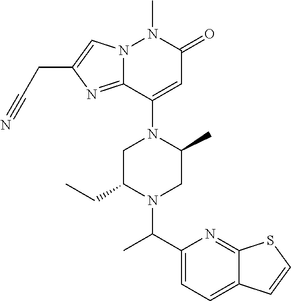 [SMILES: CC[C@@H]1CN(c2cc(=O)n(C)n3cc(CC#N)nc23)[C@@H](C)CN1C(C)c1ccc2ccsc2n1]

A solution of 2-(8-((2S,5R)-5-ethyl-2-methylpiperazin-1-yl)-5-methyl-6-oxo-5,6-dihydroimidazo[1,2-b]pyridazin-2-yl)acetonitrile (82 mg, 0.26 mmol), 1-(thieno[2,3-b]pyridin-6-yl)ethan-1-ol (50 mg, 0.26 mmol), (cyanomethyl)trimethylphosphonium iodide (190 mg, 0.78 mmol) and DIPEA (334 mg, 2.59 mmol) in CH₃CN (2 ml) was stirred at 100° C. overnight. The reaction was diluted with EtOAc (10 ml) and washed with brine (10 ml). The organic layer was concentrated under reduced pressure. The resulting residue was purified by flash column chromatography and then further purified to give the titled compound (56 mg) by Prep-HPLC(Method A). ¹H NMR (400 MHz, DMSO-d₆) δ 8.25 (dd, J=8.3, 3.3 Hz, 1H), 8.21 (s, 0.2H), 7.85 (d, J=4.9 Hz, 1H), 7.80 (dd, J=5.9, 4.3 Hz, 1H), 7.57 (dd, J=25.3, 8.3 Hz, 1H), 7.44-7.38 (m, 1H), 5.51 (d, J=3.7 Hz, 1H), 4.83-4.67 (m, 1H), 4.35-4.24 (m, 1H), 4.01 (d, J=8.2 Hz, 2H), 3.97 (q, J=6.6 Hz, 0.5H), 3.82 (q, J=6.6 Hz, 0.5H), 3.59 (s, 3H), 3.41 (d, J=12.6 Hz, 0.5H), 3.19 (d, J=10.3 Hz, 0.5H), 3.06 (d, J=9.7 Hz, 0.5H), 2.96-2.89 (m, 0.5H), 2.77-2.64 (m, 1H), 2.34 (d, J=8.4 Hz, 0.5H), 2.09 (d, J=11.8 Hz, 0.5H), 1.64-1.40 (m, 2H), 1.34 (dd, J=14.7, 6.6 Hz, 3H), 1.23 (d, J=6.4 Hz, 1.5H), 1.11 (d, J=6.4 Hz, 1.5H), 0.99 (t, J=7.3 Hz, 1.5H), 0.66 (t, J=7.3 Hz, 1.5H) ppm. MS: M/e 476 (M+1)⁺.

Compound A87: 2-(8-((2S,5R)-5-ethyl-2-methyl-4-(1-(2-methylthieno[2,3-b]pyridin-6-yl)ethyl)piperazin-1-yl)-5-methyl-6-oxo-5,6-dihydroimidazo[1,2-b]pyridazin-2-yl)acetonitrile

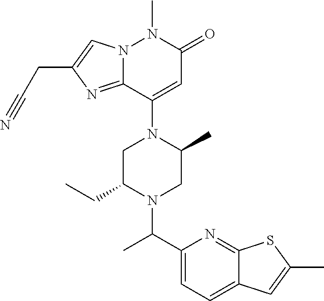 [SMILES: CC[C@@H]1CN(c2cc(=O)n(C)n3cc(CC#N)nc23)[C@@H](C)CN1C(C)c1ccc2cc(C)sc2n1]

### Step A: 2-methylthieno[2,3-b]pyridine

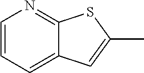

To a solution of thieno[2,3-b]pyridine (800 mg, 5.93 mmol) in THF (15 ml) at −60° C. under N₂, was added n-BuLi (1.6 M, 4.1 ml, 6.56 mmol) dropwise and stirred for 20 min. Mel (1 g, 7.04 mmol) was added to the above solution at −60° C. The reaction was warmed to RT naturally and stirred at RT for 2 hrs. The solution was quenched with brine (20 ml) and then extracted with EA (10 ml×2). The organic layer was dried and concentrated. The resulting residue was purified by flash column chromatography with 0-5% EA in PE to give the titled compound (260 mg, 29%). MS: M/e 150 (M+1)⁺.

### Step B: 2-methylthieno[2,3-b]pyridine 7-oxide

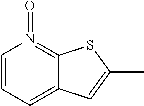

A solution of 2-methylthieno[2,3-b]pyridine (260 mg, 1.74 mmol) and mCPBA (604 mg, 3.49 mmol) in DCM (10 ml) was stirred at RT overnight. The reaction was washed with brine (10 ml×2), dried over Na₂SO₄ and concentrated to dryness. The resulting residue was purified by flash column chromatography with 0-5% MeOH in DCM to give the titled compound (200 mg, 69%). MS: M/e 166 (M+1)⁺.

### Step C: 2-methylthieno[2,3-b]pyridine-6-carbonitrile

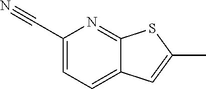

A solution of 2-methylthieno[2,3-b]pyridine 7-oxide (220 mg, 1.33 mmol), TMSCN (396 mg, 4 mmol), dimethylcarbamic chloride (432 mg, 4 mmol) and TEA (404 mg, 4 mmol) in MeCN (4 ml) was stirred at 70° C. for 3 hrs. The solution was diluted with EtOAc (15 ml), washed with brine (10 ml×2), dried over Na₂SO₄ and concentrated to dryness. The resulting residue was purified by flash column chromatography with 0-20% EA in PE to give the titled compound (175 mg, 75%). MS: M/e 175 (M+1)⁺.

### Step D: 1-(2-methylthieno[2,3-b]pyridin-6-yl)ethan-1-one

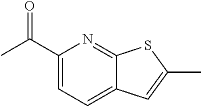

A solution of 2-methylthieno[2,3-b]pyridine-6-carbonitrile (200 mg, 1.15 mmol), MeMgBr (1M, 1.73 ml, 1.73 mmol) and CuBr (16.5 mg, 0.115 mmol) in THF (4 ml) under N₂ was stirred at 60° C. overnight. The solution was diluted with EtOAc (10 ml), washed with brine (10 ml×2), dried over Na₂SO₄ and concentrated to dryness. The resulting residue was purified by flash column chromatography with 0-15% EA in PE to give the titled compound (100 mg, 46%). MS: M/e 192 (M+1)⁺.

### Step E: 1-(2-methylthieno[2,3-b]pyridin-6-yl)ethan-1-ol

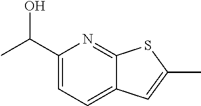

A solution of 1-(2-methylthieno[2,3-b]pyridin-6-yl)ethan-1-one (100 mg, 0.52 mmol) and NaBH₄ (20 mg, 0.53 mmol) in MeOH (2 ml) was stirred at 0° C. for 15 min. The solution was diluted with EtOAc (10 ml), washed with brine (10 ml×2), dried over Na₂SO₄ and concentrated to dryness to give the titled compound (100 mg, 99%), which was used directly for the next step without further purification. MS: M/e 194 (M+1)⁺.

### Step F: 2-(8-((2S,5R)-5-ethyl-2-methyl-4-(1-(2-methylthieno[2,3-b]pyridin-6-yl)ethyl)piperazin-1-yl)-5-methyl-6-oxo-5,6-dihydroimidazo[1,2-b]pyridazin-2-yl)acetonitrile

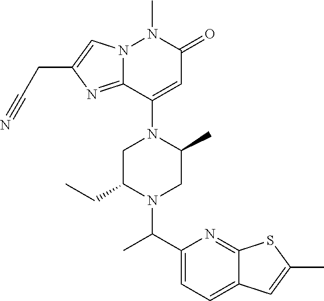 [SMILES: CC[C@@H]1CN(c2cc(=O)n(C)n3cc(CC#N)nc23)[C@@H](C)CN1C(C)c1ccc2cc(C)sc2n1]

A solution of 2-(8-((2S,5R)-5-ethyl-2-methylpiperazin-1-yl)-5-methyl-6-oxo-5,6-dihydroimidazo[1,2-b]pyridazin-2-yl)acetonitrile (82 mg, 0.26 mmol), 1-(2-methylthieno[2,3-b]pyridin-6-yl)ethan-1-ol (50 mg, 0.26 mmol), (cyanomethyl)trimethylphosphonium iodide (190 mg, 0.78 mmol) and DIPEA (334 mg, 2.59 mmol) in CH₃CN (2 ml) was stirred at 100° C. overnight. The reaction was diluted with EtOAc (10 ml) and washed with brine (10 ml). The organic layer was concentrated under reduced pressure. The resulting residue was purified by flash column chromatography and further purified by Prep-HPLC(Method A) to give the titled compound (49 mg). ¹H NMR (400 MHz, DMSO-d₆) δ 8.30 (s, 0.1H), 8.07 (dd, J=8.2, 3.4 Hz, 1H), 7.85 (d, J=4.9 Hz, 1H), 7.49 (dd, J=26.5, 8.3 Hz, 1H), 7.08 (dd, J=4.5, 1.2 Hz, 1H), 5.51 (d, J=2.6 Hz, 1H), 4.78-4.72 (m, 1H), 4.40-4.24 (m, 1H), 4.01 (d, J=7.9 Hz, 2H), 3.93 (q, J=6.7 Hz, 0.5H), 3.77 (q, J=6.6 Hz, 0.5H), 3.59 (s, 3H), 3.40 (d, J=12.3 Hz, 0.5H), 3.17 (d, J=10.6 Hz, 0.5H), 3.04 (d, J=10.8 Hz, 0.5H), 2.94-2.87 (m, 0.5H), 2.73-2.65 (m, 1H), 2.56 (s, 3H), 2.33 (d, J=9.2 Hz, 0.5H), 2.09 (d, J=9.3 Hz, 0.5H), 1.62-1.39 (m, 2H), 1.32 (dd, J=15.6, 6.6 Hz, 3H), 1.23 (d, J=6.4 Hz, 1.5H), 1.11 (d, J=6.4 Hz, 1.5H), 0.98 (t, J=7.3 Hz, 1.5H), 0.65 (t, J=7.3 Hz, 1.5H) ppm. MS: M/e 490 (M+1)⁺.

Compound A88: 2-(8-((2S,5R)-2,5-diethyl-4-(1-(4-(1-fluorocyclopropyl)phenyl)ethyl)piperazin-1-yl)-5-methyl-6-oxo-5,6-dihydroimidazo[1,2-b]pyridazin-2-yl)acetonitrile

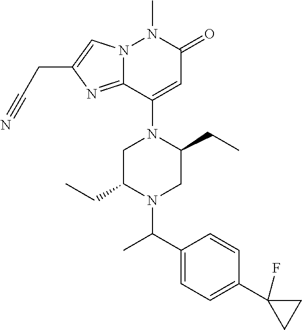 [SMILES: CC[C@H]1CN(C(C)c2ccc(C3(F)CC3)cc2)[C@H](CC)CN1c1cc(=O)n(C)n2cc(CC#N)nc12]

### Step A: 1-bromo-4-(2-bromo-1-fluoroethyl)benzene

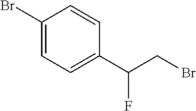 [SMILES: FC(CBr)c1ccc(Br)cc1]

To a solution of 1-bromo-4-vinylbenzene (1.8 g, 10 mol) in DCM (10 mL) at 0° C. was added Et₃N.3HF (4.8 g, 30 mol) and NBS (2.1 g, 12 mmol). The reaction mixture was slowly warmed up to RT and stirred for 16 hrs. The mixture was added H₂O (20 ml) and extracted with EA (20 mL×3). The organic phase was dried with Na₂SO₄ and concentrated by using a rotary evaporator to give a residue. The resulting residue was purified by flash column chromatography to give the titled compound (1.13 g, 40%). ¹H NMR (400 MHz, CDCl₃) δ 7.54 (t, J=9.9 Hz, 2H), 7.26 (dd, J=16.1, 8.2 Hz, 2H), 5.65-5.07 (m, 1H), 4.07-3.54 (m, 2H) ppm.

### Step B: 1-bromo-4-(1-fluorovinyl)benzene

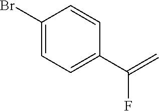

To a solution oft-BuOK (0.9 g, 8 mol) in heptane (20 mL) at 0° C. was solowly added 1-bromo-4-(2-bromo-1-fluoroethyl)benzene (1.13 g, 4 mol). The reaction mixture was warmed up to RT and stirred for 2.5 hrs. The mixture was added H₂O (20 ml) and extracted with EA (20 mL×3). The organic phase was dried with Na₂SO₄ and concentrated by using a rotary evaporator to give a residue. The resulting residue was purified by flash column chromatography to give the titled compound (800 mg, 100%). ¹H NMR (400 MHz, CDCl₃) δ 7.51 (d, J=5.0 Hz, 2H), 7.43 (s, 2H), 5.11-4.87 (m, 2H) ppm.

### Step C: 1-bromo-4-(1-fluorocyclopropyl)benzene

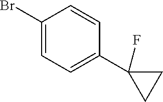 [SMILES: FC1(c2ccc(Br)cc2)CC1]

To a solution of Et₂Zn (1 M, 3 ml, 3 mol) in dry DCM (4 mL) at 0° C. was slowly added TFA (342 mg, 3 mol) by dropwise. After the addition, the reaction mixture was stirred at 0° C. for 20 mins. CH₂12 (804 mg, 3 mmol) in dry DCM (3 ml) was slowly added to the mixture by dropwise and the reaction mixture was continue stirred at 0° C. for 20 mins. 1-bromo-4-(1-fluorovinyl)benzene (300 mg, 1.5 mmol) in dry DCM (3 ml) was slowly added to the mixture by dropwise. After the addition, the reaction was slowly warmed up to RT and stirred overnight. The mixture was added H₂O (10 ml) and extracted with EA (20 mL×3). The organic phase was dried with Na₂SO₄ and concentrated by using a rotary evaporator to give a residue. The resulting residue was purified by flash column chromatography to give the titled compound (100 mg, 31%). ¹H NMR (400 MHz, CDCl₃) δ 7.48 (d, J=8.1 Hz, 2H), 7.13 (d, J=8.3 Hz, 2H), 1.50 (m, 2H), 1.05 (m, 2H) ppm.

### Step D: 1-(4-(1-fluorocyclopropyl)phenyl)ethan-1-one

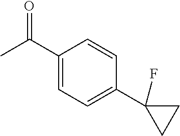

To a solution of 1-bromo-4-(1-fluorocyclopropyl)benzene (100 mg, 0.467 mol) in toluene (10 mL) was added Pd(PPh₃)₂Cl₂ (35 mg, 0.05 mol) and tributyl(1-ethoxyvinyl)stannane (255 g, 0.7 mmol). The reaction mixture was protected by N₂ atmosphere and stirred at 90° C. for 16 hrs. The mixture was added 4N HCl in dionxane (0.5 ml) and stirred at RT for 15 mins. The mixture was added saturated NaHCO₃ aqueous solution (10 ml) and extracted with EA (20 mL×3). The organic phase was dried with Na₂SO₄ and concentrated by using a rotary evaporator to give a residue. The resulting residue was purified by flash column chromatography to give the titled compound (40 mg, 48%). MS: M/e 179 (M+1)⁺.

### Step E: 1-(4-(1-fluorocyclopropyl)phenyl)ethan-1-ol

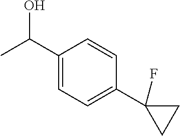

To a solution of 1-(4-(1-fluorocyclopropyl)phenyl)ethan-1-one (40 mg, 0.22 mol) in MeOH was added NaBH₄ (19 mg, 0.5 mmol). The reaction mixture was stirred at RT for 15 mins. The mixture was added H₂O (20 ml) and extracted with DCM (20 mL×3). The organic phase was dried with Na₂SO₄ and concentrated by using a rotary evaporator to give a residue. The resulting residue was purified by flash column chromatography to give the titled compound (25 mg, 63%). MS: M/e 181 (M+1)⁺.

### Step F: 2-(8-((2S,5R)-2,5-diethyl-4-(1-(4-(1-fluorocyclopropyl)phenyl)ethyl)piperazin-1-yl)-5-methyl-6-oxo-5,6-dihydroimidazo[1,2-b]pyridazin-2-yl)acetonitrile

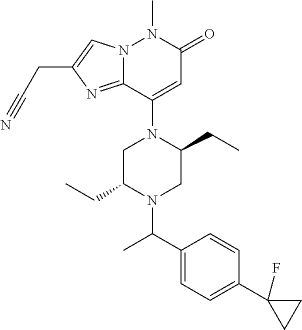 [SMILES: CC[C@H]1CN(C(C)c2ccc(C3(F)CC3)cc2)[C@H](CC)CN1c1cc(=O)n(C)n2cc(CC#N)nc12]

To a solution of 2-(8-((2S,5R)-2,5-diethylpiperazin-1-yl)-5-methyl-6-oxo-5,6-dihydroimidazo[1,2-b]pyridazin-2-yl)acetonitrile (30 mg, 0.1 mmol), 1-(4-(1-fluorocyclopropyl)phenyl)ethan-1-ol (25 mg, 0.14 mmol) and (cyanomethyl)trimethylphosphonium iodide (120 mg, 0.5 mmol) in CH₃CN (10 mL) was added DIPEA (258 mg, 2 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by Prep-HPLC(Method B) to give the titled compound (2 mg, 4%).¹H NMR (400 MHz, CD₃OD) δ 7.79 (d, J=2.1 Hz, 1H), 7.38 (dd, J=11.9, 8.1 Hz, 2H), 7.25 (m, 2H), 5.60 (s, 1H), 3.93 (d, J=3.6 Hz, 2H), 3.74 (d, J=9.0 Hz, 4H), 3.6-3.47 (m, 1H), 3.25 (s, 1H), 3.13-2.99 (m, 1H), 2.89-2.62 (m, 1H), 2.39 (m, 1H), 2.10 (m, 1H), 1.85 (m, 1H), 1.56 (m, 3H), 1.43 (dt, J=19.0, 5.4 Hz, 2H), 1.32 (dd, J=13.9, 6.5 Hz, 3H), 1.02 (m, 5H), 0.7-0.6 (m, 3H) ppm. MS: M/e 491 (M+1)⁺.

Compound A89: 2-(8-((2S,5R)-4-(1-(6-cyclopropylpyridin-3-yl)propyl)-2,5-diethylpiperazin-1-yl)-5-methyl-6-oxo-5,6-dihydroimidazo[1,2-b]pyridazin-2-yl)acetonitrile

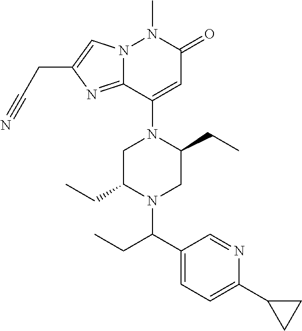 [SMILES: CCC(c1ccc(C2CC2)nc1)N1C[C@H](CC)N(c2cc(=O)n(C)n3cc(CC#N)nc23)C[C@H]1CC]

### Step A: 6-cyclopropylnicotinaldehyde

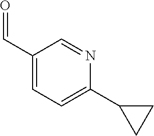 [SMILES: O=Cc1ccc(C2CC2)nc1]

To a solution of 6-bromonicotinaldehyde (1.86 g, 10 mmol) and cyclopropylboronic acid (1.72 g, 20 mmol) in toluene/H₂O (15 mL/1.5 mL) were added dichlorobis(tricyclohexylphosphine)palladium(II) (74 mg, 0.1 mmol) and K₃PO₄ (3.18 g, 15 mmol). The reaction mixture was stirred at 100° C. under N₂ overnight. The mixture was cooled to room temperature, diluted with water (40 mL), extracted with EA (80 mL×2), washed with brine, dried over Na₂SO₄, filtered, and concentrated to dryness. The residue was purified by flash column chromatography to give the titled compound (1.2 g, 81%). MS: M/e 148 (M+1)⁺.

### Step B: 1-(6-cyclopropylpyridin-3-yl)propan-1-ol

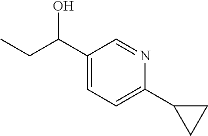

To a solution of 6-cyclopropylnicotinaldehyde (200 mg, 1.36 mmol) in THF (5 mL) was added a solution of EtMgBr in THF (0.5 mL, 3 mol/L). The reaction mixture was stirred at room temperature for 2 hrs. The mixture was diluted with saturated NH₄Cl (20 mL), extracted with EA (70 mL), washed with brine, dried over Na₂SO₄, filtered, and concentrated to dryness. The residue was purified by flash column chromatography to give the titled compound (200 mg, 83%). MS: M/e 178 (M+1)⁺.

### Step C: 2-(8-((2S,5R)-4-(1-(6-cyclopropylpyridin-3-yl)propyl)-2,5-diethylpiperazin-1-yl)-5-methyl-6-oxo-5,6-dihydroimidazo[1,2-b]pyridazin-2-yl)acetonitrile

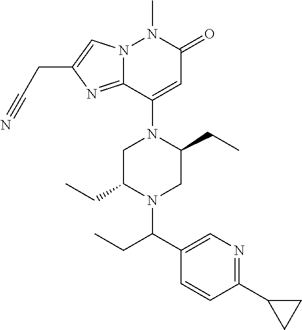 [SMILES: CCC(c1ccc(C2CC2)nc1)N1C[C@H](CC)N(c2cc(=O)n(C)n3cc(CC#N)nc23)C[C@H]1CC]

To a solution of 1-(6-cyclopropylpyridin-3-yl)propan-1-ol (35 mg, 0.2 mmol), 2-(8-((2S,5R)-2,5-diethylpiperazin-1-yl)-5-methyl-6-oxo-5,6-dihydroimidazo[1,2-b]pyridazin-2-yl)acetonitrile (33 mg, 0.1 mmol) and (cyanomethyl) trimethyl phosphonium iodide (73 mg, 0.3 mmol) in CH₃CN (1 mL) was added DIPEA (129 mg, 1 mmol). The mixture was sealed in a bottle and heated at 100° C. for 16 hours. The mixture was cooled to room temperature, diluted with water, extracted with EA (80 mL×2), washed with brine, dried over Na₂SO₄, filtered, and concentrated to dryness. The resulting residue was purified by combi flash (DCM/MeOH=20/1) and Prep-HPLC(Method A) to give the titled Compound A89a (1.5 mg) and Compound A89b (1.5 mg, 6.1%).

Compound A89a (the earlier peak): ¹H NMR (400 MHz, CD₃OD) δ 8.24 (d, J=1.6 Hz, 1H), 7.78 (s, 1H), 7.67 (dd, J=2.0 Hz, 8.0 Hz, 1H), 7.23 (d, J=8.4 Hz, 1H), 5.58 (s, 1H), 3.93 (s, 2H), 3.73 (s, 3H), 3.59-3.50 (m, 1H), 3.35-3.20 (m, 3H), 2.98-2.80 (m, 2H), 2.42-2.32 (m, 1H), 2.16-1.94 (m, 3H), 1.86-1.59 (m, 2H), 1.53-1.42 (m, 2H), 1.09-1.01 (m, 2H), 0.99-0.91 (m, 5H), 0.71 (t, J=7.2 Hz, 6H) ppm. MS: M/e 488 (M+1)⁺.

Compound A89a (the later peak): ¹H NMR (400 MHz, CD₃OD) δ 8.28 (d, J=1.6 Hz, 1H), 7.79 (s, 1H), 7.68 (dd, J=2.0 Hz, 8.4 Hz, 1H), 7.20 (d, J=8.4 Hz, 1H), 5.59 (s, 1H), 3.93 (s, 2H), 3.73 (s, 3H), 3.51-3.38 (m, 2H), 3.35-3.31 (m, 2H), 3.19-3.10 (m, 1H), 2.76-2.62 (m, 1H), 2.34-2.26 (m, 1H), 2.14-2.03 (m, 1H), 2.00-1.83 (m, 2H), 1.75-1.40 (m, 4H), 1.10-0.85 (m, 7H), 0.67 (t, J=7.6 Hz, 3H), 0.61 (t, J=7.2 Hz, 3H) ppm. MS: M/e 488 (M+1)⁺.

Compound A90: 2-(8-((2S,5R)-4-(1-(6-(1,1-difluoroethyl)pyridin-3-yl)ethyl)-2,5-diethylpiperazin-1-yl)-5-methyl-6-oxo-5,6-dihydroimidazo [1,2-b]pyridazin-2-yl)acetonitrile

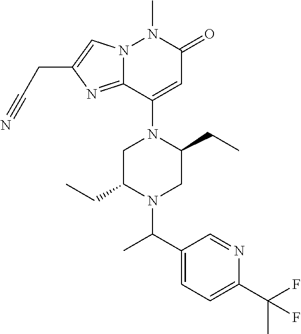 [SMILES: CC[C@H]1CN(C(C)c2ccc(C(C)(F)F)nc2)[C@H](CC)CN1c1cc(=O)n(C)n2cc(CC#N)nc12]

### Step A: 5-bromo-2-(1,1-difluoroethyl)pyridine

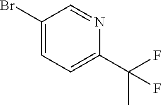

1-(5-bromopyridin-2-yl)ethan-1-one was dissolved in BAST (2.21 g, 10 mmol), then MeOH (1 drop) was added. After the addition, the reaction mixture was stirred overnight at 70° C. The reaction was quenched with aq.NaHCO₃, then extracted with EtOAc (20 mL×3). The combined organic layers were washed with brine, dried over Na₂SO₄, concentrated and purified by flash column chromatography to give the titled compound (0.4 g, 36%). MS: M/e 222/224 (M+1)⁺.

### Step B: 1-(6-(1,1-difluoroethyl)pyridin-3-yl)ethan-1-one

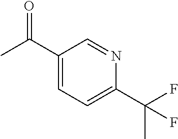

A mixture of 5-bromo-2-(1,1-difluoroethyl)pyridine (400 mg, 1.8 mmol), tributyl(1-ethoxyvinyl)stannane (813 mg, 2.7 mmol) and Pd(PPh₃)₂Cl₂ (126 mg, 0.18 mmol) in toluene (10 mL) was stirred at 100° C. overnight under N₂. The reaction mixture was treated with aq.HCl (2.0 M, 5 mL) and stirred for an hour, then extracted with EtOAc (15 mL×3). The combined organic layers were washed with brine, dried over Na₂SO₄, concentrated and purified by flash column chromatography to give the titled compound (304 mg, 91%). MS: M/e 186 (M+1)⁺.

### Step C: 1-(6-(1,1-difluoroethyl)pyridin-3-yl)ethan-1-ol

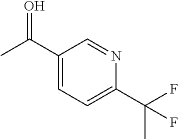

To a stirred solution of 1-(6-(1,1-difluoroethyl)pyridin-3-yl)ethan-1-one (304 mg, 1.64 mmol) in MeOH (10 mL) was added NaBH₄ (62 mg, 1.64 mmol). After then, the mixture was stirred for 10 min. The reaction mixture was poured into H₂O (10 mL) and most MeOH was removed to give the aqueous layer, then extracted with EtOAc (10 mL×2). The combined organic layers were washed with brine, dried over Na₂SO₄, concentrated to give the titled compound (258 mg, 84%). MS: M/e 188 (M+1)⁺.

### Step D: 2-(8-((2S,5R)-4-(1-(6-(1,1-difluoroethyl)pyridin-3-yl)ethyl)-2,5-diethylpiperazin-1-yl)-5-methyl-6-oxo-5,6-dihydroimidazo[1,2-b]pyridazin-2-yl)acetonitrile

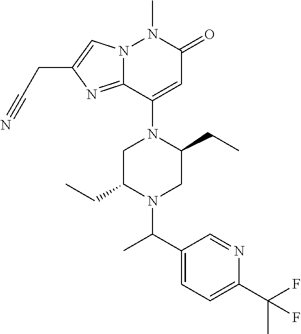 [SMILES: CC[C@H]1CN(C(C)c2ccc(C(C)(F)F)nc2)[C@H](CC)CN1c1cc(=O)n(C)n2cc(CC#N)nc12]

A mixture of 2-(8-((2S,5R)-2,5-diethylpiperazin-1-yl)-5-methyl-6-oxo-5,6-dihydroimidazo[1,2-b]pyridazin-2-yl)acetonitrile (32.8 mg, 0.1 mmol), 1-(6-(1,1-difluoroethyl)pyridin-3-yl)ethan-1-ol (37 mg, 0.2 mmol), (cyanomethyl)trimethylphosphonium iodide (72.9 mg, 0.3 mmol) and DIPEA (64.5 mg, 0.5 mmol) in CH₃CN (3 mL) was stirred overnight at 100° C. in a sealed tube. The reaction mixture was diluted with EtOAc (20 mL), washed with H₂O, brine, dried over Na₂SO₄, concentrated to dryness. The resulting residue was purified and separated into Compound A90a (5 mg) and Compound A90b (6 mg) by Prep-HPLC (Method A).

Compound A90a (the earlier peak): ¹H NMR (400 MHz, CD₃OD) δ 8.60 (s, 1H), 7.99 (dd, J=8.0, 1.6 Hz, 1H), 7.78 (s, 1H), 7.70 (d, J=8.0 Hz, 1H), 5.61 (s, 1H), 3.98-3.89 (m, 3H), 3.73 (s, 3H), 3.35-3.31 (m, 2H), 3.30-3.27 (m, 1H), 3.04-2.88 (m, 2H), 2.42-2.33 (m, 1H), 2.16-2.05 (m, 1H), 1.99 (t, J=18.4 Hz, 3H), 1.90-1.78 (m, 1H), 1.63-1.50 (m, 2H), 1.39 (d, J=6.4 Hz, 3H), 0.94 (t, J=7.2 Hz, 3H), 0.74 (t, J=7.2 Hz, 3H) ppm. MS: M/e 498 (M+1)⁺.

Compound A90b (the later peak): ¹H NMR (400 MHz, CD₃OD) δ 8.63 (s, 1H), 8.00 (dd, J=8.0, 2.0 Hz, 1H), 7.79 (s, 1H), 7.67 (d, J=8.0 Hz, 1H), 5.60 (s, 1H), 3.94 (s, 2H), 3.81-3.75 (m, 1H), 3.73 (s, 3H), 3.55-3.48 (m, 1H), 3.34-3.31 (m, 2H), 3.22-3.14 (m, 1H), 2.76-2.69 (m, 1H), 2.32-2.25 (m, 1H), 1.97 (t, J=18.4 Hz, 3H), 1.95-1.85 (m, 1H), 1.75-1.51 (m, 3H), 1.36 (d, J=6.4 Hz, 3H), 1.04 (t, J=7.2 Hz, 3H), 0.62 (t, J=7.2 Hz, 3H) ppm. MS: M/e 498 (M+1)⁺.

Compound A91: 2-(8-((2S,5R)-2,5-diethyl-4-(1-(4-(trifluoromethyl)phenyl)ethyl)piperazin-1-yl)-5-methyl-6-oxo-5,6-dihydroimidazo[1,2-b]pyridazin-2-yl)acetonitrile

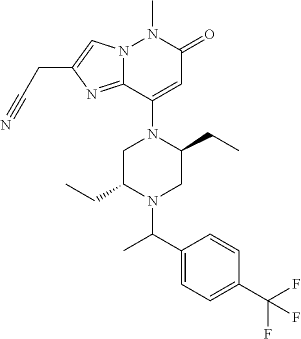 [SMILES: CC[C@H]1CN(C(C)c2ccc(C(F)(F)F)cc2)[C@H](CC)CN1c1cc(=O)n(C)n2cc(CC#N)nc12]

### Step A: 1-(4-(trifluoromethyl)phenyl)ethan-1-ol

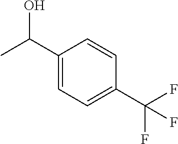

To a solution of 4-(trifluoromethyl)benzaldehyde (870 mg, 5 mmol) in THF (5 mL) was added methylmagnesium bromide (3 M in ether, 2 ml, 6 mmol) at 0° C. and the mixture was stirred at room temperature for 30 minutes. The resulting mixture was treated with saturated NH₄Cl (30 ml), extracted with EtOAc (10 mL×2). The combined organic layers were dried over Na₂SO₄ and concentrated to dryness. The resulting residue was purified by silica gel column chromatography EtOAc:PE=0-80% in 25 minutes to give the titled compound (500 mg, 52%). MS: M/e 191 (M+1)⁺.

### Step B: 2-(8-((2S,5R)-2,5-diethyl-4-(1-(4-(trifluoromethyl)phenyl)ethyl)piperazin-1-yl)-5-methyl-6-oxo-5,6-dihydroimidazo[1,2-b]pyridazin-2-yl)acetonitrile

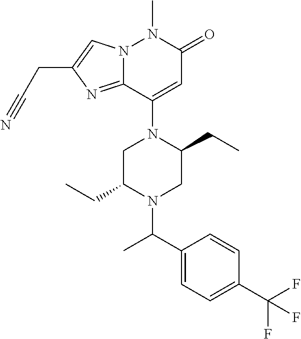 [SMILES: CC[C@H]1CN(C(C)c2ccc(C(F)(F)F)cc2)[C@H](CC)CN1c1cc(=O)n(C)n2cc(CC#N)nc12]

A mixture of 1-(4-(trifluoromethyl)phenyl)ethan-1-ol (114 mg, 0.6 mmol), 2-(8-((2S,5R)-2,5-diethylpiperazin-1-yl)-5-methyl-6-oxo-5,6-dihydroimidazo[1,2-b]pyridazin-2-yl)acetonitrile (100 mg, 0.3 mmol), (cyanomethyl) trimethylphosphonium iodide (290 mg, 2.44 mmol) and DIPEA (155 mg, 1.2 mmol) in MeCN (3 mL) was stirred at 100° C. overnight. The reaction mixture was diluted with water (20 mL), extracted with EtOAc (10 mL×2), dried over Na₂SO₄ and concentrated to dryness. The resulting residue was purified and separated into Compound A91a (18 mg) and Compound A91b (27 mg) by Prep-HPLC(Method A).

Compound A91a (the earlier peak): ¹H NMR (400 MHz, CDCl₃) δ 7.59 (d, J=8.1 Hz, 2H), 7.46 (d, J=8.0 Hz, 2H), 7.34 (s, 1H), 5.59 (s, 1H), 3.82-3.74 (m, 3H), 3.71 (s, 3H), 3.21 (d, J=10.6 Hz, 1H), 3.00-2.92 (m, 1H), 2.84 (d, J=9.8 Hz, 1H), 2.35 (d, J=9.9 Hz, 1H), 2.19-2.02 (m, 1H), 1.83-1.68 (m, 1H), 1.64-1.40 (m, 4H), 1.32 (d, J=6.5 Hz, 3H), 0.93 (t, J=7.2 Hz, 3H), 0.69 (t, J=7.2 Hz, 3H) ppm. MS: M/e 501 (M+1)⁺.

Compound A91a (the later peak):¹H NMR (400 MHz, CDCl₃) δ 7.59 (d, J=8.1 Hz, 2H), 7.46 (d, J=8.0 Hz, 2H), 7.34 (s, 1H), 5.59 (s, 1H), 4.88-4.05 (m, 1H), 3.78 (s, 2H), 3.71 (s, 3H), 3.62 (q, J=6.5 Hz, 1H), 3.43 (d, J=11.5 Hz, 1H), 3.08 (d, J=10.1 Hz, 1H), 2.63 (d, J=9.2 Hz, 1H), 2.27 (d, J=12.1 Hz, 1H), 1.89 (s, 1H), 1.66 (td, J=13.7, 7.1 Hz, 2H), 1.49 (dd, J=13.5, 7.2 Hz, 2H), 1.29 (d, J=6.5 Hz, 3H), 0.99 (t, J=7.2 Hz, 3H), 0.60 (t, J=7.4 Hz, 3H) ppm. MS: M/e 501 (M+1)⁺.uangbin

Compound A92: 2-(8-((2S,5R)-2,5-diethyl-4-(1-(2-fluoro-4-(trifluoromethyl)phenyl)ethyl)piperazin-1-yl)-5-methyl-6-oxo-5,6-dihydroimidazo[1,2-b]pyridazin-2-yl)acetonitrile

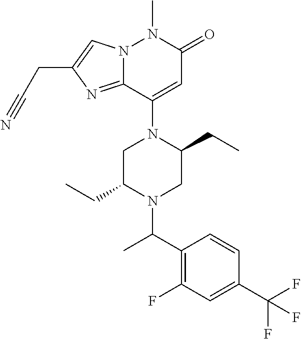 [SMILES: CC[C@H]1CN(C(C)c2ccc(C(F)(F)F)cc2F)[C@H](CC)CN1c1cc(=O)n(C)n2cc(CC#N)nc12]

### Step A: 1-(2-fluoro-4-(trifluoromethyl)phenyl)ethan-1-ol

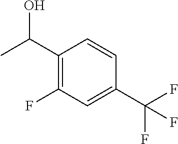

To a solution of 1-(2-fluoro-4-(trifluoromethyl)phenyl)ethan-1-one (1 g, 5 mmol) in MeOH (10 mL) was added NaBH₄ (153 mg, 4 mmol) at room temperature and the mixture was stirred at room temperature for 30 minutes. The resulting mixture was treated with water (50 ml), extracted with EtOAc (15 mL×2). The combined organic layers were dried over Na₂SO₄ and concentrated to dryness. The resulting residue was purified by flash column chromatography (EtOAc:PE=0-100% in 20 minutes) to give the titled compound (900 mg, 86%). MS: M/e 209 (M+1)⁺.

### Step B: 2-(8-((2S,5R)-2,5-diethyl-4-(1-(2-fluoro-4-(trifluoromethyl)phenyl)ethyl)piperazin-1-yl)-5-methyl-6-oxo-5,6-dihydroimidazo[1,2-b]pyridazin-2-yl)acetonitrile

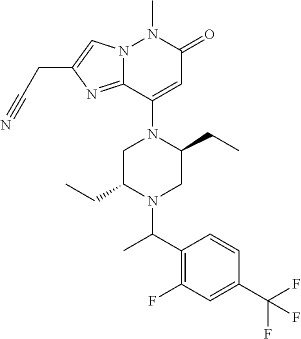 [SMILES: CC[C@H]1CN(C(C)c2ccc(C(F)(F)F)cc2F)[C@H](CC)CN1c1cc(=O)n(C)n2cc(CC#N)nc12]

A mixture of 1-(2-fluoro-4-(trifluoromethyl)phenyl)ethan-1-ol (125 mg, 0.6 mmol), 2-(8-((2S,5R)-2,5-diethylpiperazin-1-yl)-5-methyl-6-oxo-5,6-dihydroimidazo[1,2-b]pyridazin-2-yl)acetonitrile (100 mg, 0.3 mmol), (cyanomethyl) trimethylphosphonium iodide (290 mg, 2.44 mmol) and DIPEA (155 mg, 1.2 mmol) in MeCN (3 mL) was stirred at 100° C. overnight. The reaction mixture was diluted with water (20 mL), extracted with EtOAc (10 mL×2), dried over Na₂SO₄ and concentrated to dryness. The resulting residue was purified and separated into Compound A92a (28 mg) and Compound A92b (42 mg) by Prep-HPLC (Method A).

Compound A92a (the earlier peak): ¹H NMR (400 MHz, CDCl₃) δ 7.65 (t, J=7.4 Hz, 1H), 7.42 (d, J=8.0 Hz, 1H), 7.37-7.28 (m, 2H), 5.60 (s, 1H), 4.57 (s, 1H), 4.21 (q, J=6.5 Hz, 1H), 3.77 (s, 2H), 3.72 (s, 3H), 3.22 (d, J=12.5 Hz, 1H), 2.90 (q, J=11.7 Hz, 2H), 2.36 (d, J=8.9 Hz, 1H), 2.06 (dd, J=14.2, 6.6 Hz, 1H), 1.83-1.68 (m, 1H), 1.66-1.42 (m, 3H), 1.34 (d, J=6.5 Hz, 3H), 0.91 (t, J=7.1 Hz, 3H), 0.74 (t, J=7.2 Hz, 3H) ppm. MS: M/e 519 (M+1)⁺.

Compound A92b (the later peak): ¹H NMR (400 MHz, CDCl₃) δ 7.74 (t, J=7.3 Hz, 1H), 7.40 (d, J=8.1 Hz, 1H), 7.35 (s, 1H), 7.30 (s, 1H), 5.60 (s, 1H), 4.05 (d, J=6.3 Hz, 1H), 3.78 (s, 2H), 3.72 (s, 3H), 3.43 (d, J=12.4 Hz, 1H), 3.07 (d, J=10.0 Hz, 1H), 2.69 (d, J=9.7 Hz, 1H), 2.27 (d, J=12.2 Hz, 1H), 1.79-1.65 (m, 4H), 1.56-1.44 (m, 2H), 1.30 (d, J=6.2 Hz, 3H), 1.00 (s, 3H), 0.63 (s, 3H) ppm. MS: M/e 519 (M+1)⁺.

Compound A93: 2-(8-((2S,5R)-4-(1-(6-cyclopropylpyridin-3-yl)propyl)-2-ethyl-5-methylpiperazin-1-yl)-5-methyl-6-oxo-5,6-dihydroimidazo[1,2-b]pyridazin-2-yl)acetonitrile

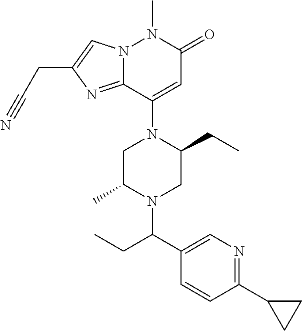 [SMILES: CCC(c1ccc(C2CC2)nc1)N1C[C@H](CC)N(c2cc(=O)n(C)n3cc(CC#N)nc23)C[C@H]1C]

### Step A: 6-cyclopropylnicotinaldehyde

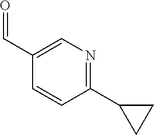 [SMILES: O=Cc1ccc(C2CC2)nc1]

To a solution of 6-bromonicotinaldehyde (1.86 g, 10 mmol) and cyclopropylboronic acid (1.72 g, 20 mmol) in toluene/H₂O (15 mL/1.5 mL) were added dichlorobis(tricyclohexylphosphine)palladium(II) (74 mg, 0.1 mmol) and K₃PO₄ (3.18 g, 15 mmol). The reaction mixture was stirred at 90° C. under N₂ overnight. The mixture was cooled to room temperature, diluted with water (40 mL), extracted with EA (60 mL×2), washed with brine, dried over Na₂SO₄, filtered, and concentrated to dryness. The residue was purified by flash column chromatography to give the titled compound (1.2 g, 81%). MS: M/e 148 (M+1)⁺.

### Step B: 1-(6-cyclopropylpyridin-3-yl)propan-1-ol

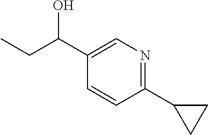

To a solution of 6-cyclopropylnicotinaldehyde (200 mg, 1.36 mmol) in THF (5 mL) was added a solution of CH₃CH₂MgBr in ether (0.5 mL, 3 mol/L, 1.5 mmol) slowly at 0° C. The resulting mixture was stirred at room temperature for 2 hour. The reaction mixture was quenched with saturated NH₄Cl (3 mL) and H₂O (10 mL) and extracted with EA (50 mL×2), washed with brine, dried over Na₂SO₄, filtered, and concentrated to dryness. The residue was purified by flash column chromatography to give the titled compound (200 mg, 83%). MS: M/e 178 (M+1)⁺.

### Step C: 2-(8-((2S,5R)-4-(1-(6-cyclopropylpyridin-3-yl)propyl)-2-ethyl-5-methylpiperazin-1-yl)-5-methyl-6-oxo-5,6-dihydroimidazo[1,2-b]pyridazin-2-yl)acetonitrile

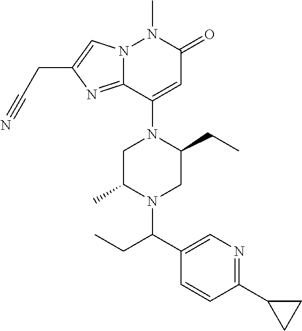 [SMILES: CCC(c1ccc(C2CC2)nc1)N1C[C@H](CC)N(c2cc(=O)n(C)n3cc(CC#N)nc23)C[C@H]1C]

To a solution of 1-(6-cyclopropylpyridin-3-yl)propan-1-ol (35 mg, 0.2 mmol), 2-(8-((2S,5R)-2-ethyl-5-methylpiperazin-1-yl)-5-methyl-6-oxo-5,6-dihydroimidazo[1,2-b]pyridazin-2-yl)acetonitrile (33 mg, 0.1 mmol) and (cyanomethyl) trimethyl phosphonium iodide (73 mg, 0.6 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄, filtered, and concentrated to dryness. The resulting residue was purified and separated into Compound A93a (2 mg) and Compound A93b (1 mg) by Prep-HPLC(Method A).

Compound A93a (the earlier peak): ¹H NMR (400 MHz, CD₃OD) δ 8.27 (d, J=1.6 Hz, 1H), 7.78 (m, 1H), 7.68 (dd, J=2.0 Hz, 7.6 Hz, 1H), 7.20 (d, J=8.4 Hz, 1H), 5.59 (s, 1H), 3.94 (s, 2H), 3.73 (s, 3H), 3.66-3.52 (m, 2H), 3.39-3.31 (m, 3H), 2.76-2.64 (m, 1H), 2.32-2.22 (m, 1H), 2.15-1.80 (m, 3H), 1.75-1.50 (m, 2H), 1.11 (d, J=6.4 Hz, 3H), 1.08-0.85 (m, 4H), 0.73-0.58 (m, 6H) ppm. MS: M/e 474 (M+1)⁺.

Compound A93b (the later peak): ¹H NMR (400 MHz, CD₃OD) δ 8.27 (d, J=1.6 Hz, 1H), 7.78 (m, 1H), 7.67 (dd, J=2.0 Hz, 7.6 Hz, 1H), 7.22 (d, J=8.0 Hz, 1H), 5.59 (s, 1H), 3.94 (s, 2H), 3.73 (s, 3H), 3.52-3.35 (m, 2H), 3.34-3.31 (m, 3H), 3.05-2.75 (m, 3H), 2.15-1.79 (m, 4H), 1.70-1.50 (m, 1H), 1.10-0.86 (m, 9H), 0.69 (t, J=7.6 Hz, 3H) ppm. MS: M/e 474 (M+1)⁺.

Compound A94: 2-(8-((2S,5R)-2,5-diethyl-4-(1-(2-methylbenzo[d]thiazol-6-yl)ethyl)piperazin-1-yl)-5-(methyl-d₃)-6-oxo-5,6-dihydroimidazo[1,2-b]pyridazin-2-yl)acetonitrile

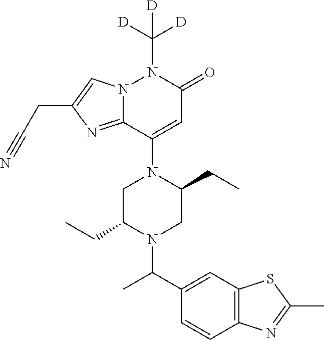 [SMILES: [2H]C([2H])([2H])n1c(=O)cc(N2C[C@@H](CC)N(C(C)c3ccc4nc(C)sc4c3)C[C@@H]2CC)c2nc(CC#N)cn21]

### Step A: methyl-d₃ 8-((2S,5R)-4-(tert-butoxycarbonyl)-2,5-diethylpiperazin-1-yl)-5-(methyl-d₃)-6-oxo-5,6-dihydroimidazo[1,2-b]pyridazine-2-carboxylate

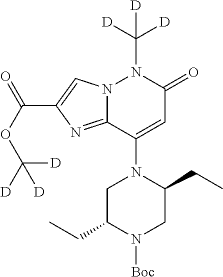 [SMILES: [2H]C([2H])([2H])OC(=O)c1cn2c(n1)c(N1C[C@@H](CC)N(C(=O)OC(C)(C)C)C[C@@H]1CC)cc(=O)n2C([2H])([2H])[2H]]

A mixture of 8-((2S,5R)-4-(tert-butoxycarbonyl)-2,5-diethylpiperazin-1-yl)-6-oxo-5,6-dihydroimidazo[1,2-b]pyridazine-2-carboxylic acid (2.09 g, 5 mmol), CD₃I (3.63 g, 25 mmol) and Cs₂CO₃ (4.87 g, 15 mmol) in dioxane (20 mL) was stirred at 80° C. overnight in a sealed tube. The reaction mixture was filtered. The filtrate was concentrated and purified by flash column chromatography to give the titled compound (1.05 g, 46%). MS: M/e 454 (M+1)⁺.

### Step B: tert-butyl (2R,5S)-2,5-diethyl-4-(2-(hydroxymethyl)-5-(methyl-d₃)-6-oxo-5,6-dihydroimidazo[1,2-b]pyridazin-8-yl)piperazine-1-carboxylate

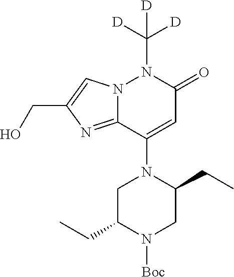 [SMILES: [2H]C([2H])([2H])n1c(=O)cc(N2C[C@@H](CC)N(C(=O)OC(C)(C)C)C[C@@H]2CC)c2nc(CO)cn21]

To a stirred solution of methyl-d₃ 8-((2S,5R)-4-(tert-butoxycarbonyl)-2,5-diethylpiperazin-1-yl)-5-(methyl-d₃)-6-oxo-5,6-dihydroimidazo[1,2-b]pyridazine-2-carboxylate (1.05 g, 2.31 mmol) in EtOH/THF (5 mL/10 mL) was added NaBH₄ (438 mg, 11.55 mmol). After the addition, the reaction was stirred at 60° C. overnight. The reaction mixture was diluted with saturated citric acid solution (5 mL) and H₂O (30 mL), extracted with DCM (60 mL×2). The combined organic layers were washed with brine (40 mL), dried over Na₂SO₄, concentrated. The residue was purified by flash column chromatography to give the titled compound (0.9 g, 92%). MS: M/e 423 (M+1)⁺.

### Step C: tert-butyl (2R,5S)-4-(2-(chloromethyl)-5-(methyl-d₃)-6-oxo-5,6-dihydroimidazo[1,2-b]pyridazin-8-yl)-2,5-diethylpiperazine-1-carboxylate

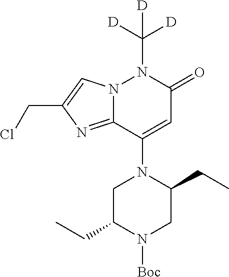 [SMILES: [2H]C([2H])([2H])n1c(=O)cc(N2C[C@@H](CC)N(C(=O)OC(C)(C)C)C[C@@H]2CC)c2nc(CCl)cn21]

To a stirred solution of tert-butyl (2R,5S)-2,5-diethyl-4-(2-(hydroxymethyl)-5-(methyl-d₃)-6-oxo-5,6-dihydroimidazo[1,2-b]pyridazin-8-yl)piperazine-1-carboxylate (0.91 g, 2.15 mmol) in CH₂Cl₂ (10 mL) was added SOCl₂ (282 mg, 2.37 mmol). After the addition, the reaction was stirred for 20 min. The reaction mixture was washed with aq.NaHCO₃, brine, dried over Na₂SO₄, filtered and concentrated to give the titled compound (1 g, crude). MS: M/e 441 (M+1)⁺.

### Step D: tert-butyl (2R,5S)-4-(2-(cyanomethyl)-5-(methyl-d₃)-6-oxo-5,6-dihydroimidazo[1,2-b]pyridazin-8-yl)-2,5-diethylpiperazine-1-carboxylate

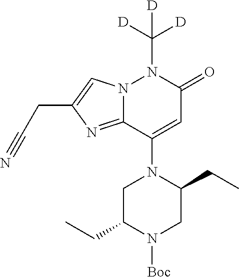 [SMILES: [2H]C([2H])([2H])n1c(=O)cc(N2C[C@@H](CC)N(C(=O)OC(C)(C)C)C[C@@H]2CC)c2nc(CC#N)cn21]

To a stirred solutiong of tert-butyl (2R,5S)-4-(2-(chloromethyl)-5-(methyl-d₃)-6-oxo-5,6-dihydroimidazo[1,2-b]pyridazin-8-yl)-2,5-diethylpiperazine-1-carboxylate (1 g, 2.15 mmol) in CH₂Cl₂ (10 mL) was added TMSCN (0.43 g, 4.3 mmol), followed by TBAF (1.0 M, 4.3 mL, 4.3 mmol). After the addition, the reaction mixture was stirred overnight. The reaction mixture was concentrated and purified by flash column chromatography to give the titled compound (820 mg, 88% for two steps). MS: M/e 432 (M+1)⁺.

### Step E: 2-(8-((2S,5R)-2,5-diethylpiperazin-1-yl)-5-(methyl-d₃)-6-oxo-5,6-dihydroimidazo[1,2-b]pyridazin-2-yl)acetonitrile

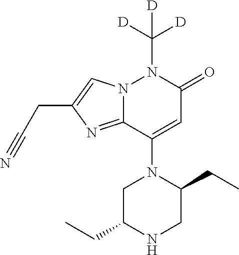 [SMILES: [2H]C([2H])([2H])n1c(=O)cc(N2C[C@@H](CC)NC[C@@H]2CC)c2nc(CC#N)cn21]

To a stirred solution of tert-butyl (2R,5S)-4-(2-(cyanomethyl)-5-(methyl-d₃)-6-oxo-5,6-dihydroimidazo[1,2-b]pyridazin-8-yl)-2,5-diethylpiperazine-1-carboxylate (0.82 g, 1.9 mmol) in CH₂Cl₂ (10 mL) was added TMSOTf (0.84 g, 3.8 mmol). After the addition, the reaction was stirred for 3 hours. The reaction mixture was quenched with aq.NaHCO₃, then extracted with CH₂Cl₂/IPA (3/1, 40 mL×3). The combined organic layers were dried over Na₂SO₄, concentrated to give the titled compound (0.43 g, 68%). MS: M/e 332 (M+1)⁺

### Step F: 2-(8-((2S,5R)-2,5-diethyl-4-(1-(2-methylbenzo[d]thiazol-6-yl)ethyl)piperazin-1-yl)-5-(methyl-d₃)-6-oxo-5,6-dihydroimidazo[1,2-b]pyridazin-2-yl)acetonitrile

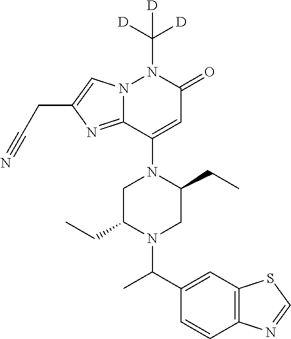 [SMILES: [2H]C([2H])([2H])n1c(=O)cc(N2C[C@@H](CC)N(C(C)c3ccc4ncsc4c3)C[C@@H]2CC)c2nc(CC#N)cn21]

To a solution of 1-(2-methylbenzo[d]thiazol-6-yl)ethan-1-ol (40 mg, 0.2 mmol), 2-(8-((2S,5R)-2,5-diethylpiperazin-1-yl)-5-(methyl-d₃)-6-oxo-5,6-dihydroimidazo[1,2-b]pyridazin-2-yl)acetonitrile (33 mg, 0.1 mmol) and (cyanomethyl) trimethyl phosphonium iodide (72 mg, 0.3 mmol) in CH₃CN (1 mL) was added DIPEA (64 mg, 0.5 mmol). The mixture was sealed in a bottle and heated at 100° C. for 16 hours. The mixture was cooled to room temperature, diluted with water, extracted with EA (80 mL×2), washed with brine, dried over Na₂SO₄, filtered, and concentrated to dryness. The resulting residue was purified by Prep-TLC (EtOAc) and further separated into Compound A94a (4 mg) and Compound A94b (6 mg) by Prep-HPLC (Method A).

Compound A94a (the earlier peak): ¹H NMR (400 MHz, CD₃OD) δ 7.92 (s, 1H), 7.86 (d, J=8.4 Hz, 1H), 7.77 (s, 1H), 7.53 (dd, J=1.6 Hz, 8.0 Hz, 1H), 5.60 (s, 1H), 3.91 (s, 2H), 3.90-3.81 (m, 1H), 3.35-3.31 (m, 2H), 3.28-3.21 (m, 1H), 3.07-3.00 (m, 1H), 2.95-2.87 (m, 1H), 2.82 (s, 3H), 2.48-2.37 (m, 1H), 2.20-2.05 (m, 1H), 1.92-1.77 (m, 1H), 1.65-1.44 (m, 2H), 1.40 (d, J=6.0 Hz, 3H), 0.96 (t, J=7.2 Hz, 3H), 0.67 (t, J=7.2 Hz, 3H) ppm. MS: M/e 507 (M+1)⁺.

Compound A94b (the later peak): ¹H NMR (400 MHz, CD₃OD) δ 7.95 (s, 1H), 7.83 (d, J=8.0 Hz, 1H), 7.77 (s, 1H), 7.55 (d, J=8.4 Hz, 1H), 5.58 (s, 1H), 3.92 (s, 2H), 3.79-3.70 (m, 1H), 3.56-3.44 (m, 1H), 3.35-3.31 (m, 2H), 3.22-3.11 (m, 1H), 2.82 (s, 3H), 2.71-2.61 (m, 1H), 2.40-2.32 (m, 1H), 2.06-1.90 (m, 1H), 1.75-1.50 (m, 3H), 1.36 (d, J=6.8 Hz, 3H), 1.04 (t, J=7.2 Hz, 3H), 0.57 (t, J=7.2 Hz, 3H) ppm. MS: M/e 507(M+1)⁺.

Compound A95: 2-(8-((2S,5R)-2,5-diethyl-4-(1-(2-methylbenzo[d]thiazol-6-yl)ethyl-2,2,2-d₃)piperazin-1-yl)-5-(methyl-d₃)-6-oxo-5,6-dihydroimidazo[1,2-b]pyridazin-2-yl)acetonitrile

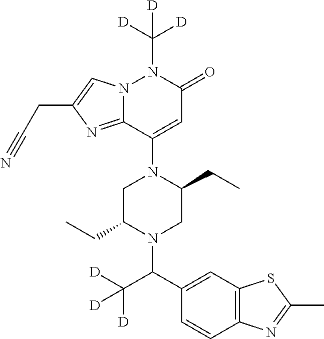 [SMILES: [2H]C([2H])([2H])C(c1ccc2nc(C)sc2c1)N1C[C@H](CC)N(c2cc(=O)n(C([2H])([2H])[2H])n3cc(CC#N)nc23)C[C@H]1CC]

### Step A: 1-(2-methylbenzo[d]thiazol-6-yl)ethan-2,2,2-d₃-1-ol

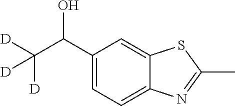 [SMILES: [2H]C([2H])([2H])C(O)c1ccc2nc(C)sc2c1]

To a solution of 2-methylbenzo[d]thiazole-6-carbaldehyde (180 mg, 1 mmol) in THF (5 mL) was added a solution of CD₃MgI in THF (1.2 mL, 1 mol/L). The reaction mixture was stirred at room temperature for 2 hrs. The mixture was diluted with saturated NH₄Cl (20 mL), extracted with EA (60 mL×2), washed with brine, dried over Na₂SO₄, filtered, and concentrated to dryness. The residue was purified by flash column chromatography to give the titled compound (180 mg, 91%). MS: M/e 197 (M+1)⁺.

### Step B: 2-(8-((2S,5R)-2,5-diethyl-4-(1-(2-methylbenzo[d]thiazol-6-yl)ethyl-2,2,2-d₃)piperazin-1-yl)-5-(methyl-d₃)-6-oxo-5,6-dihydroimidazo[1,2-b]pyridazin-2-yl)acetonitrile

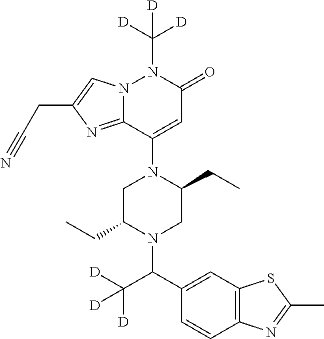 [SMILES: [2H]C([2H])([2H])C(c1ccc2nc(C)sc2c1)N1C[C@H](CC)N(c2cc(=O)n(C([2H])([2H])[2H])n3cc(CC#N)nc23)C[C@H]1CC]

To a solution of 1-(2-methylbenzo[d]thiazol-6-yl)ethan-2,2,2-d₃-1-ol (40 mg, 0.2 mmol), 2-(8-((2S,5R)-2,5-diethylpiperazin-1-yl)-5-(methyl-d₃)-6-oxo-5,6-dihydroimidazo[1,2-b]pyridazin-2-yl)acetonitrile (33 mg, 0.1 mmol) and (cyanomethyl) trimethyl phosphonium iodide (72 mg, 0.3 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (80 mL×2), washed with brine, dried over Na₂SO₄, filtered, and concentrated to dryness. The resulting residue was purified and by Prep-TLC (EA) and further separated into Compound A95a (3 mg) and Compound A95b (3 mg) by Prep-HPLC (Method A).

Compound A95a (the earlier peak): ¹H NMR (400 MHz, CD₃OD) δ 7.86 (d, J=0.8 Hz, 1H), 7.85 (d, J=8.8 Hz, 1H), 7.77 (s, 1H), 7.53 (dd, J=1.2 Hz, 8.0 Hz, 1H), 5.60 (s, 1H), 3.91 (s, 2H), 3.91-3.81 (m, 1H), 3.35-3.31 (m, 2H), 3.28-3.21 (m, 1H), 3.06-2.98 (m, 1H), 2.94-2.87 (m, 1H), 2.82 (s, 3H), 2.46-2.37 (m, 1H), 2.16-2.05 (m, 1H), 1.90-1.77 (m, 1H), 1.60-1.44 (m, 2H), 0.95 (t, J=7.2 Hz, 3H), 0.67 (t, J=7.2 Hz, 3H) ppm. MS: M/e 510 (M+1)⁺.

Compound A95b (the later peak): ¹H NMR (400 MHz, CD₃OD) δ 7.95 (s, 1H), 7.83 (d, J=8.4 Hz, 1H), 7.78 (s, 1H), 7.55 (dd, J=0.8 Hz, 8.0 Hz, 1H), 5.58 (s, 1H), 3.93 (s, 2H), 3.87-3.71 (m, 1H), 3.55-3.44 (m, 1H), 3.35-3.31 (m, 2H), 3.22-3.11 (m, 1H), 2.82 (s, 3H), 2.73-2.62 (m, 1H), 2.40-2.30 (m, 1H), 2.05-1.90 (m, 1H), 1.75-1.50 (m, 3H), 1.04 (t, J=7.2 Hz, 3H), 0.57 (t, J=7.2 Hz, 3H) ppm. MS: M/e 510(M+1)⁺.

Compound A96: 2-(8-((2S,5R)-2,5-diethyl-4-(1-(2-(methyl-d₃)benzo[d]thiazol-6-yl)ethyl)piperazin-1-yl)-5-methyl-6-oxo-5,6-dihydroimidazo [1,2-b]pyridazin-2-yl)acetonitrile

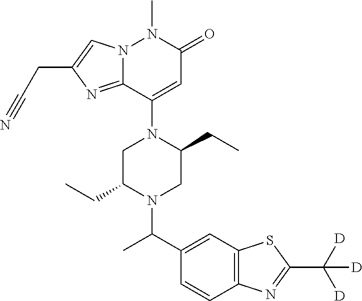 [SMILES: [2H]C([2H])([2H])c1nc2ccc(C(C)N3C[C@H](CC)N(c4cc(=O)n(C)n5cc(CC#N)nc45)C[C@H]3CC)cc2s1]

### Step A: 1-(2-(methyl-d₃)benzo[d]thiazol-6-yl)ethan-1-ol

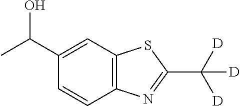 [SMILES: [2H]C([2H])([2H])c1nc2ccc(C(C)O)cc2s1]

A solution of 1-(2-methylbenzo[d]thiazol-6-yl)ethan-1-ol (4386 mg, 2 mmol) in D₂O (2 ml) and DMSO-d₆ (2 ml) was added tBuOK (450 mg, 4 mol). The reaction was stirred at room temperature for 16 hours. The reaction was diluted with water (30 ml), extracted with EA (60 mL×2), washed with brine (30 ml), dried over Na₂SO₄ and concentrated. The residue was purified by flash column chromatography with 30-60% EA in PE to give the titled compound (330 mg, 76%). MS: M/e 197 (M+1)⁺.

### Step B: 2-(8-((2S,5R)-2,5-diethyl-4-(1-(2-(methyl-d₃)benzo[d]thiazol-6-yl)ethyl)piperazin-1-yl)-5-methyl-6-oxo-5,6-dihydroimidazo[1,2-b]pyridazin-2-yl)acetonitrile

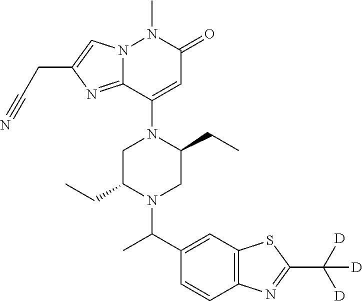 [SMILES: [2H]C([2H])([2H])c1nc2ccc(C(C)N3C[C@H](CC)N(c4cc(=O)n(C)n5cc(CC#N)nc45)C[C@H]3CC)cc2s1]

To a solution of 1-(2-(methyl-d₃)benzo[d]thiazol-6-yl)ethan-1-ol (40 mg, 0.2 mmol), 2-(8-((2S,5R)-2,5-diethylpiperazin-1-yl)-5-methyl-6-oxo-5,6-dihydroimidazo[1,2-b]pyridazin-2-yl)acetonitrile (31 mg, 0.1 mmol) and (cyanomethyl) trimethyl phosphonium iodide (72 mg, 0.3 mmol) in CH₃CN (1 mL) was added DIPEA (64 mg, 0.5 mmol). The mixture was sealed in a bottle and heated at 100° C. for 16 hours. The mixture was cooled to room temperature, diluted with water, extracted with EA (80 mL), washed with brine, dried over Na₂SO₄, filtered, and concentrated to dryness. The resulting residue was purified by Prep-TLC (EA) and further separated into Compound A96a (6 mg) and Compound A96b (9 mg) by Prep-HPLC (Method A).

Compound A96a (the earlier peak): ¹H NMR (400 MHz, CD₃OD) δ 7.93 (s, 1H), 7.86 (d, J=8.0 Hz, 1H), 7.78 (s, 1H), 7.54 (d, J=8.4 Hz, 1H), 5.61 (s, 1H), 3.92 (s, 2H), 3.90-3.85 (m, 1H), 3.73 (s, 3H), 3.35-3.31 (m, 2H), 3.29-3.24 (m, 1H), 3.08-3.00 (m, 1H), 2.96-2.87 (m, 1H), 2.48-2.38 (m, 1H), 2.20-2.05 (m, 1H), 1.92-1.77 (m, 1H), 1.63-1.45 (m, 2H), 1.40 (d, J=6.8 Hz, 3H), 0.96 (t, J=7.2 Hz, 3H), 0.68 (t, J=7.2 Hz, 3H) ppm. MS: M/e 507 (M+1)⁺.

Compound A96b (the later peak): ¹H NMR (400 MHz, CD₃OD) δ 7.96 (s, 1H), 7.83 (d, J=8.8 Hz, 1H), 7.79 (s, 1H), 7.56 (dd, J=1.2 Hz, 8.0 Hz, 1H), 5.59 (s, 1H), 3.93 (s, 2H), 3.79-3.70 (m, 4H), 3.56-3.45 (m, 1H), 3.35-3.31 (m, 2H), 3.24-3.14 (m, 1H), 2.72-2.64 (m, 1H), 2.40-2.32 (m, 1H), 2.06-1.90 (m, 1H), 1.75-1.50 (m, 3H), 1.37 (d, J=6.8 Hz, 3H), 1.05 (t, J=7.2 Hz, 3H), 0.58 (t, J=7.2 Hz, 3H) ppm. MS: M/e 507(M+1)⁺.

Compound A97: 2-(8-((2S,5R)-2,5-diethyl-4-(1-(2-(methyl-d₃)benzo[d]thiazol-6-yl)ethyl)piperazin-1-yl)-5-(methyl-d₃)-6-oxo-5,6-dihydroimidazo[1,2-b]pyridazin-2-yl)acetonitrile

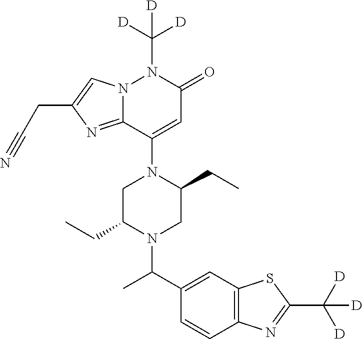 [SMILES: [2H]C([2H])([2H])c1nc2ccc(C(C)N3C[C@H](CC)N(c4cc(=O)n(C([2H])([2H])[2H])n5cc(CC#N)nc45)C[C@H]3CC)cc2s1]

To a solution of 1-(2-(methyl-d₃)benzo[d]thiazol-6-yl)ethan-1-ol (40 mg, 0.2 mmol), 2-(8-((2S,5R)-2,5-diethylpiperazin-1-yl)-5-(methyl-d₃)-6-oxo-5,6-dihydroimidazo[1,2-b]pyridazin-2-yl)acetonitrile (33 mg, 0.1 mmol) and (cyanomethyl) trimethyl phosphonium iodide (72 mg, 0.3 mmol) in CH₃CN (1 mL) was added DIPEA (64 mg, 0.5 mmol). The mixture was sealed in a bottle and heated at 100° C. for 16 hours. The mixture was cooled to room temperature, diluted with water, extracted with EA (80 mL), washed with brine, dried over Na₂SO₄, filtered, and concentrated to dryness. The resulting residue was purified by Prep-TLC (EA) and further separated into Compound A97a (4 mg) and Compound A97b (6 mg) by Prep-HPLC (Method A).

Compound A97a (the earlier peak): ¹H NMR (400 MHz, CD₃OD) δ 7.92 (s, 1H), 7.85 (d, J=8.4 Hz, 1H), 7.77 (s, 1H), 7.53 (d, J=8.8 Hz, 1H), 5.60 (s, 1H), 3.91 (s, 2H), 3.90-3.85 (m, 1H), 3.35-3.31 (m, 2H), 3.29-3.24 (m, 1H), 3.08-3.00 (m, 1H), 2.96-2.87 (m, 1H), 2.48-2.38 (m, 1H), 2.20-2.05 (m, 1H), 1.92-1.77 (m, 1H), 1.63-1.45 (m, 2H), 1.39 (d, J=6.4 Hz, 3H), 0.95 (t, J=7.2 Hz, 3H), 0.67 (t, J=7.2 Hz, 3H) ppm. MS: M/e 510 (M+1)⁺.

Compound A97b (the later peak): ¹H NMR (400 MHz, CD₃OD) δ 7.96 (s, 1H), 7.83 (d, J=8.8 Hz, 1H), 7.78 (s, 1H), 7.56 (dd, J=1.2 Hz, 8.4 Hz, 1H), 5.59 (s, 1H), 3.93 (s, 2H), 3.81-3.70 (m, 1H), 3.56-3.45 (m, 1H), 3.35-3.31 (m, 2H), 3.24-3.14 (m, 1H), 2.72-2.64 (m, 1H), 2.40-2.32 (m, 1H), 2.06-1.90 (m, 1H), 1.75-1.50 (m, 3H), 1.37 (d, J=6.8 Hz, 3H), 1.05 (t, J=7.2 Hz, 3H), 0.58 (t, J=7.2 Hz, 3H) ppm. MS: M/e 510(M+1)⁺.

Compound A98: 2-(8-((2S,5R)-2-ethyl-5-methyl-4-(1-(2-methylthiazolo[5,4-b]pyridin-5-yl)propyl)piperazin-1-yl)-5-methyl-6-oxo-5,6-dihydroimidazo[1,2-b]pyridazin-2-yl)acetonitrile

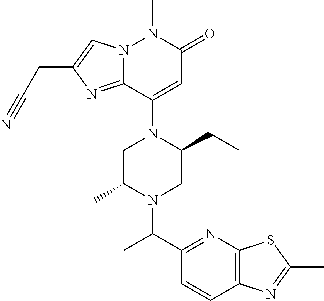 [SMILES: CC[C@H]1CN(C(C)c2ccc3nc(C)sc3n2)[C@H](C)CN1c1cc(=O)n(C)n2cc(CC#N)nc12]

### Step A: ethyl 8-((2S,5R)-4-(tert-butoxycarbonyl)-2-ethyl-5-methylpiperazin-1-yl)-6-chloroimidazo[1,2-b]pyridazine-2-carboxylate

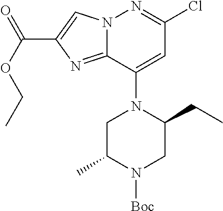 [SMILES: CCOC(=O)c1cn2nc(Cl)cc(N3C[C@@H](C)N(C(=O)OC(C)(C)C)C[C@@H]3CC)c2n1]

A mixture of the ethyl 8-bromo-6-chloroimidazo[1,2-b]pyridazine-2-carboxylate (6.4 g, 20.9 mmol), tert-butyl (2R,5S)-5-ethyl-2-methylpiperazine-1-carboxylate (5 g, 21.9 mmol) and DIPEA (5.4 g, 41.8 mmol) in CH₃CN (25 mL) was stirred at 100° C. overnight in a sealed tube. The reaction mixture was poured into H₂O (200 mL), extracted with EtOAc (150 mL×2). The combined organic layers were washed with brine, dried over Na₂SO₄, and concentrated. The resulting residue was purified by flash column chromatography to give the titled compound (9.4 g, 100%). MS: M/e 452 (M+1)⁺.

### Step B: 8-((2S,5R)-4-(tert-butoxycarbonyl)-2-ethyl-5-methylpiperazin-1-yl)-6-oxo-5,6-dihydroimidazo[1,2-b]pyridazine-2-carboxylic acid

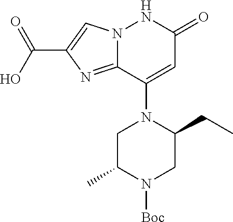 [SMILES: CC[C@H]1CN(C(=O)OC(C)(C)C)[C@H](C)CN1c1cc(=O)[nH]n2cc(C(=O)O)nc12]

To a stirred solution of the product of ethyl 8-((2S,5R)-4-(tert-butoxycarbonyl)-2-ethyl-5-methylpiperazin-1-yl)-6-chloroimidazo[1,2-b]pyridazine-2-carboxylate (9.4 g, 23.2 mmol) in dioxane (200 mL) was added aq.KOH (7.8 g, 0.139 mol, in 50 mL H₂O), then Pd₂(dba)₃ (2.12 g, 2.32 mmol) and t-BuXPhOS (1.97 g, 4.64 mmol) were added under N₂. After the addition, the reaction mixture was stirred at 100° C. for 3 hours. The reaction mixture was poured into H₂O (100 mL) and extracted with EtOAc (100 mL×2). The combined organic layers were discarded and the aqueous layer was acidified to pH=3-4 with aq.HCl acid, and extracted with EtOAc (150 mL×4). The combined organic layers were washed with brine, dried over Na₂SO₄, concentrated to give the target compound (8.8 g, 94%). MS: M/e 406 (M+1)⁺.

### Step C: methyl 8-((2S,5R)-4-(tert-butoxycarbonyl)-2-ethyl-5-methylpiperazin-1-yl)-5-methyl-6-oxo-5,6-dihydroimidazo[1,2-b]pyridazine-2-carboxylate

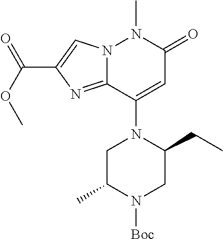 [SMILES: CC[C@H]1CN(C(=O)OC(C)(C)C)[C@H](C)CN1c1cc(=O)n(C)n2cc(C(=O)OC)nc12]

A mixture of 8-((2S,5R)-4-(tert-butoxycarbonyl)-2-ethyl-5-methylpiperazin-1-yl)-6-oxo-5,6-dihydroimidazo[1,2-b]pyridazine-2-carboxylic acid (8 g, 19.75 mmol), Mel (16.8 g, 118.5 mmol) and Cs₂CO₃ (19.3 g, 59.25 mmol) in dioxane (200 mL) was stirred at 80° C. overnight in a sealed tube. The reaction mixture was filtered and the filtrate was concentrated and purified by flash column chromatography to give the titled compound (3.9 g, 46%). MS: M/e 434 (M+1)⁺.

### Step D: tert-butyl (2R,5S)-5-ethyl-4-(2-(hydroxymethyl)-5-methyl-6-oxo-5,6-dihydroimidazo[1,2-b]pyridazin-8-yl)-2-methylpiperazine-1-carboxylate

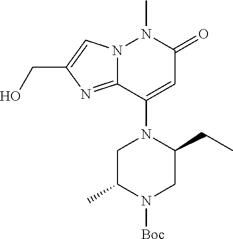 [SMILES: CC[C@H]1CN(C(=O)OC(C)(C)C)[C@H](C)CN1c1cc(=O)n(C)n2cc(CO)nc12]

To a stirred solution of methyl 8-((2S,5R)-4-(tert-butoxycarbonyl)-2-ethyl-5-methylpiperazin-1-yl)-5-methyl-6-oxo-5,6-dihydroimidazo[1,2-b]pyridazine-2-carboxylate (3.9 g, 8.96 mmol) in EtOH/THF (20 mL/40 mL) was added NaBH₄ (2.04 g, 53.8 mmol). After the addition, the reaction was stirred at 70° C. overnight. The reaction mixture was diluted with H₂O (80 mL), extracted with EtOAc (15 mL×4). The combined organic layers were washed with brine, dried over Na₂SO₄, concentrated to give the titled compound (3.8 g, 100%). MS: M/e 406 (M+1)⁺.

### Step E: tert-butyl (2R,5S)-4-(2-(chloromethyl)-5-methyl-6-oxo-5,6-dihydroimidazo[1,2-b]pyridazin-8-yl)-5-ethyl-2-methylpiperazine-1-carboxylate

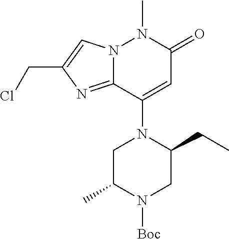 [SMILES: CC[C@H]1CN(C(=O)OC(C)(C)C)[C@H](C)CN1c1cc(=O)n(C)n2cc(CCl)nc12]

To a stirred solution of tert-butyl (2R,5S)-5-ethyl-4-(2-(hydroxymethyl)-5-methyl-6-oxo-5,6-dihydroimidazo[1,2-b]pyridazin-8-yl)-2-methylpiperazine-1-carboxylate (3.8 g, 9.38 mmol) in CH₂Cl₂ (50 mL) was added SOCl₂ (1.26 g, 10.6 mmol). After the addition, the reaction was stirred for 20 min. The reaction mixture was washed with aq.NaHCO₃, brine, dried over Na₂SO₄, concentrated to give the titled compound (3.8 g, 96%). MS: M/e 424 (M+1)⁺.

### Step F: tert-butyl (2R,5S)-4-(2-(cyanomethyl)-5-methyl-6-oxo-5,6-dihydroimidazo[1,2-b]pyridazin-8-yl)-5-ethyl-2-methylpiperazine-1-carboxylate

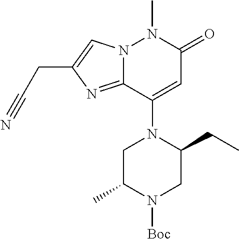 [SMILES: CC[C@H]1CN(C(=O)OC(C)(C)C)[C@H](C)CN1c1cc(=O)n(C)n2cc(CC#N)nc12]

To a stirred solutiong of tert-butyl (2R,5S)-4-(2-(chloromethyl)-5-methyl-6-oxo-5,6-dihydroimidazo[1,2-b]pyridazin-8-yl)-5-ethyl-2-methylpiperazine-1-carboxylate (3.8 g, 9.38 mmol) in CH₂Cl₂ (10 mL) was added TMSCN (1.86 g, 18.76 mmol), followed by TBAF (1.0 M, 18.76 mL, 18.76 mmol). After the addition, the reaction mixture was stirred overnight. The reaction mixture was concentrated and purified by flash column chromatography to give the titled compound (2.7 g, 69%). MS: M/e 415 (M+1)*.

### Step G: 2-(8-((2S,5R)-2-ethyl-5-methylpiperazin-1-yl)-5-methyl-6-oxo-5,6-dihydroimidazo[1,2-b]pyridazin-2-yl)acetonitrile

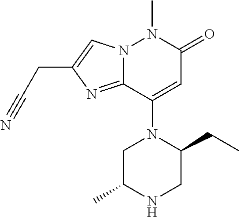 [SMILES: CC[C@H]1CN[C@H](C)CN1c1cc(=O)n(C)n2cc(CC#N)nc12]

To a stirred solution of tert-butyl (2R,5S)-4-(2-(cyanomethyl)-5-methyl-6-oxo-5,6-dihydroimidazo[1,2-b]pyridazin-8-yl)-5-ethyl-2-methylpiperazine-1-carboxylate (2.68 g, 6.47 mmol) in CH₂Cl₂ (30 mL) was added TMSOTf (2.88 g, 12.9 mmol). After the addition, the reaction was stirred for 3 hours. The reaction mixture was quenched with aq.NaHCO₃, then extracted with CH₂Cl₂/IPA (3/1, 40 mL×6). The combined organic layers were dried over Na₂SO₄, concentrated to give the titled compound (2.2 g, 100%). MS: M/e 315 (M+1)⁺

### Step H: 2-(8-((2S,5R)-2-ethyl-5-methyl-4-(1-(2-methylthiazolo[5,4-b]pyridin-5-yl)propyl)piperazin-1-yl)-5-methyl-6-oxo-5,6-dihydroimidazo[1,2-b]pyridazin-2-yl)acetonitrile

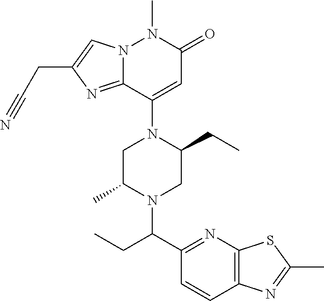 [SMILES: CCC(c1ccc2nc(C)sc2n1)N1C[C@H](CC)N(c2cc(=O)n(C)n3cc(CC#N)nc23)C[C@H]1C]

A mixture of 2-(8-((2S,5R)-2-ethyl-5-methylpiperazin-1-yl)-5-methyl-6-oxo-5,6-dihydroimidazo[1,2-b]pyridazin-2-yl)acetonitrile (31.4 mg, 0.1 mmol), 1-(2-methylthiazolo[5,4-b]pyridin-5-yl)propan-1-ol (41.6 mg, 0.2 mmol), (cyanomethyl)trimethylphosphonium iodide (72.9 mg, 0.3 mmol) and DIPEA (64.5 mg, 0.5 mmol) in CH₃CN (4 mL) was stirred at 100° C. overnight in a sealed tube. The reaction mixture was diluted with EtOAc (10 mL), washed with brine, dried over Na₂SO₄, concentrated to dryness. The resulting residue was purified and separated into Compound A98a (4 mg) and Compound A98b (6 mg) by Prep-HPLC (Method A).

Compound A98a (the earlier peak): ¹H NMR (400 MHz, CD₃OD) δ 8.23 (d, J=8.4 Hz, 1H), 7.78 (s, 1H), 7.69 (d, J=8.4 Hz, 1H), 5.60 (s, 1H), 3.93 (s, 2H), 3.73 (s, 3H), 3.71-3.59 (m, 3H), 3.34-3.31 (m, 2H), 2.92-2.83 (m, 4H), 2.31-2.23 (m, 1H), 2.16-1.64 (m, 4H), 1.14 (d, J=6.4 Hz, 3H), 0.68 (t, J=7.2 Hz, 3H), 0.62 (t, J=7.2 Hz, 3H) ppm. MS: M/e 505 (M+1)⁺.

Compound A98b (the later peak): ¹H NMR (400 MHz, CD₃OD) δ 8.23 (d, J=8.4 Hz, 1H), 7.78 (s, 1H), 7.66 (d, J=8.4 Hz, 1H), 5.58 (s, 1H), 3.93 (s, 2H), 3.83-3.76 (m, 1H), 3.73 (s, 3H), 3.47-3.39 (m, 1H), 3.35-3.31 (m, 2H), 3.09-2.97 (m, 2H), 2.90-2.81 (m, 4H), 2.10-1.76 (m, 4H), 1.00 (d, J=6.4 Hz, 3H), 0.94 (t, J=7.6 Hz, 3H), 0.71 (t, J=7.2 Hz, 3H) ppm. MS: M/e 505 (M+1)⁺.

Compound A99: 2-(8-((2S,5R)-2,5-diethyl-4-(1-(2-methylthiazolo[5,4-b]pyridin-5-yl)propyl)piperazin-1-yl)-5-methyl-6-oxo-5,6-dihydroimidazo[1,2-b]pyridazin-2-yl)acetonitrile

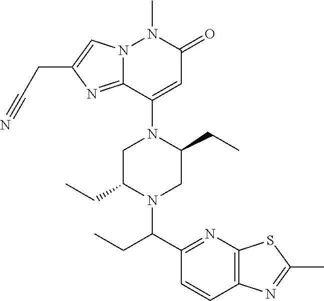 [SMILES: CCC(c1ccc2nc(C)sc2n1)N1C[C@H](CC)N(c2cc(=O)n(C)n3cc(CC#N)nc23)C[C@H]1CC]

### Step A: 2-methylthiazolo[5,4-b]pyridine-5-carbonitrile

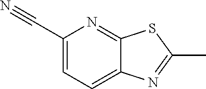

To a stirred solution of 5-chloro-2-methylthiazolo[5,4-b]pyridine (368 mg, 2 mmol) in DMF (8 mL) was added ZnCN₂ (762 mg, 6 mmol), followed by Pd(PPh₃)₄(231 mg, 0.2 mmol). After the addition, the reaction mixture was stirred overnight at 100° C. under N₂. The reaction mixture was poured into H₂O (15 mL) and extracted with EtOAc (15 mL×3). The combined organic layers were washed with brine, dried over Na₂SO₄, concentrated and purified by flash column chromatography to give the titled compound (257 mg, 73.4%). MS: M/e 176 (M+1)⁺.

### Step B: 1-(2-methylthiazolo[5,4-b]pyridin-5-yl)propan-1-one

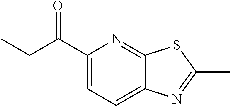

To a stirred solution of 2-methylthiazolo[5,4-b]pyridine-5-carbonitrile (387 mg, 2.2 mmol) in THF (10 mL) was added dropwise EtMgBr (3.0 M, 1.47 mL, 4.42 mmol) at 0° C. After the addition, the reaction was quenched with aq.NH₄Cl, extracted with EtOAc (40 mL×3). The combined organic layers were washed with brine, dried over Na₂SO₄, concentrated and purified by flash column chromatography to give the titled compound (90 mg, 19.8%). MS: M/e 207 (M+1)⁺.

### Step C: 1-(2-methylthiazolo[5,4-b]pyridin-5-yl)propan-1-ol

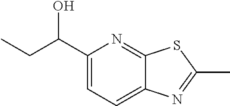

To a stirred solution of 1-(2-methylthiazolo[5,4-b]pyridin-5-yl)propan-1-one (90 mg, 0.437 mmol) in MeOH (5 mL) was added NaBH₄ (16.6 mg, 0.437 mmol). After the addition, the mixture was stirred for 10 min. The reaction was quenched with H₂O (10 mL), then most MeOH was removed to give the aqueous layer and extracted with EtOAc (10 mL×3). The combined organic layers were washed with brine, dried over Na₂SO₄, concentrated to give the titled compound (92 mg, 100%). MS: M/e 209 (M+1)⁺.

### Step D: 2-(8-((2S,5R)-2,5-diethyl-4-(1-(2-methylthiazolo[5,4-b]pyridin-5-yl)propyl)piperazin-1-yl)-5-methyl-6-oxo-5,6-dihydroimidazo[1,2-b]pyridazin-2-yl)acetonitrile

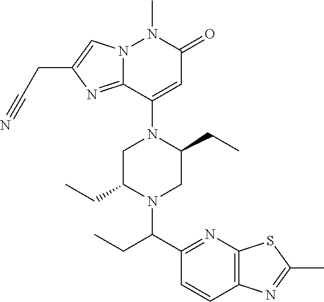 [SMILES: CCC(c1ccc2nc(C)sc2n1)N1C[C@H](CC)N(c2cc(=O)n(C)n3cc(CC#N)nc23)C[C@H]1CC]

A mixture of 2-(8-((2S,5R)-2,5-diethylpiperazin-1-yl)-5-methyl-6-oxo-5,6-dihydroimidazo[1,2-b]pyridazin-2-yl)acetonitrile (32.8 mg, 0.1 mmol), 1-(2-methylthiazolo[5,4-b]pyridin-5-yl)propan-1-ol (41.6 mg, 0.2 mmol), (cyanomethyl)trimethylphosphonium iodide (72.9 mg, 0.3 mmol) and DIPEA (64.5 mg, 0.5 mmol) in CH₃CN (4 mL) was stirred at 100° C. overnight in a sealed tube. The reaction mixture was diluted with EtOAc (10 mL), washed with brine, dried over Na₂SO₄, concentrated to dryness. The resulting residue was purified and separated into Compound A99a (4 mg) and Compound A99b (5 mg) by Prep-HPLC (Method A).

Compound A99a (the earlier peak): ¹H NMR (400 MHz, CD₃OD) δ 8.22 (d, J=8.4 Hz, 1H), 7.78 (s, 1H), 7.71 (d, J=8.4 Hz, 1H), 5.58 (s, 1H), 3.93 (s, 2H), 3.73 (s, 3H), 3.72-3.67 (m, 1H), 3.55-3.47 (m, 1H), 3.35-3.31 (m, 2H), 3.22-3.15 (m, 1H), 2.86 (s, 3H), 2.84-2.76 (m, 1H), 2.29-2.22 (m, 1H), 2.07-1.44 (m, 6H), 1.03 (t, J=7.2 Hz, 3H), 0.67 (t, J=7.2 Hz, 3H), 0.59 (t, J=7.2 Hz, 3H) ppm. MS: M/e 519 (M+1)⁺.

Compound A99b (the later peak): ¹H NMR (400 MHz, CD₃OD) δ 8.23 (d, J=8.4 Hz, 1H), 7.78 (s, 1H), 7.62 (d, J=8.4 Hz, 1H), 5.58 (s, 1H), 3.92 (s, 2H), 3.88-3.82 (m, 1H), 3.73 (s, 3H), 3.35-3.31 (m, 2H), 3.29-3.26 (m, 1H), 3.08-2.93 (m, 2H), 2.87 (s, 3H), 2.44-2.37 (m, 1H), 2.12-1.76 (m, 4H), 1.64-1.42 (m, 2H), 0.95 (t, J=7.2 Hz, 3H), 0.77-0.66 (m, 6H) ppm. MS: M/e 519 (M+1)⁺.

Compound A100: 2-(8-((2S,5R)-2,5-diethyl-4-(1-(2-ethylbenzo[d]thiazol-6-yl)ethyl)piperazin-1-yl)-5-methyl-6-oxo-5,6-dihydroimidazo[1,2-b]pyridazin-2-yl)acetonitrile

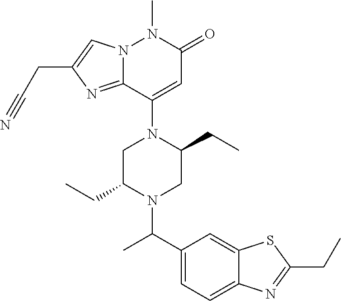 [SMILES: CCc1nc2ccc(C(C)N3C[C@H](CC)N(c4cc(=O)n(C)n5cc(CC#N)nc45)C[C@H]3CC)cc2s1]

To a solution of 2-(8-((2S,5R)-2,5-diethylpiperazin-1-yl)-5-methyl-6-oxo-5,6-dihydroimidazo[1,2-b]pyridazin-2-yl)acetonitrile (30 mg, 0.09 mmol) in CH₃CN (3 mL) and was added 1-(2-ethylbenzo[d]thiazol-6-yl)ethan-1-ol (19 mg, 0.09 mmol), (cyanomethyl)trimethylphosphonium iodide (67 mg, 0.27 mmol) and DIPEA (59 mg, 0.46 mmol). The resulting mixture was stirred at 105° C. overnight. The reaction solvent was removed under reduce pressure to afford crude product. The resulting residue was purified and separated into Compound A100a (9 mg) and Compound A100b (9 mg, 40%) by Prep-HPLC(Method A).

Compound A100a (the earlier peak): ¹H NMR (400 MHz, CD₃OD) δ 7.93 (s, 1H), 7.87 (d, J=8.3 Hz, 1H), 7.77 (s, 1H), 7.53 (d, J=8.5 Hz, 1H), 5.60 (s, 1H), 3.91 (s, 2H), 3.89 (d, J=6.4 Hz, 1H), 3.73 (s, 3H), 3.29-3.25 (m, 3H), 3.15 (d, J=7.6 Hz, 2H), 3.04 (d, J=12.1 Hz, 1H), 2.91 (d, J=12.3 Hz, 1H), 2.42 (d, J=8.9 Hz, 1H), 2.18-2.09 (m, 1H), 1.90-1.80 (m, 1H), 1.65-1.50 (m, 2H), 1.46 (t, J=7.6 Hz, 3H), 1.39 (d, J=6.4 Hz, 3H), 0.96 (t, J=7.3 Hz, 3H), 0.67 (t, J=7.3 Hz, 3H) ppm. MS: M/e 518(M+1)⁺.

Compound A100b (the later peak): ¹H NMR (400 MHz, CD₃OD) δ 7.97 (s, 1H), 7.84 (d, J=8.5 Hz, 1H), 7.78 (s, 1H), 7.56 (d, J=8.0 Hz, 1H), 5.58 (s, 1H), 3.93 (s, 2H), 3.76 (s, 1H), 3.73 (s, 3H), 3.54-3.47 (m, 1H), 3.27-3.24 (m, 1H), 3.21-3.10 (m, 4H), 2.70-2.65 (m, 1H), 2.37 (d, J=12.2 Hz, 1H), 2.04-1.96 (m, 1H), 1.72-1.58 (m, 3H), 1.45 (t, J=7.5 Hz, 3H), 1.36 (d, J=6.5 Hz, 3H), 1.05 (d, J=7.5 Hz, 3H), 0.57 (s, 3H) ppm. MS: M/e 518(M+1)⁺.

Compound A101: 2-(8-((2S,5R)-5-ethyl-4-(1-(2-ethylbenzo[d]thiazol-6-yl)ethyl)-2-methylpiperazin-1-yl)-5-methyl-6-oxo-5,6-dihydroimidazo[1,2-b]pyridazin-2-yl)acetonitrile

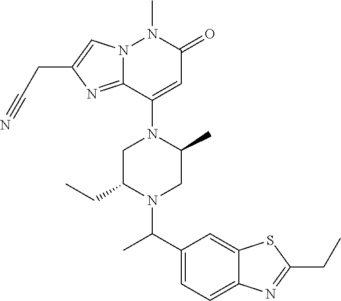 [SMILES: CCc1nc2ccc(C(C)N3C[C@H](C)N(c4cc(=O)n(C)n5cc(CC#N)nc45)C[C@H]3CC)cc2s1]

To a solution of 2-(8-((2S,5R)-5-ethyl-2-methylpiperazin-1-yl)-5-methyl-6-oxo-5,6-dihydroimidazo[1,2-b]pyridazin-2-yl)acetonitrile (30 mg, 0.10 mmol) in CH₃CN (3 mL) and was added 1-(2-ethylbenzo[d]thiazol-6-yl)ethan-1-ol (20 mg, 0.10 mmol), (cyanomethyl)trimethylphosphonium iodide (70 mg, 0.29 mmol) and DIPEA (62 mg, 0.48 mmol). The resulting mixture was stirred at 105° C. overnight. The reaction solvent was removed under reduce pressure to afford crude product. The resulting residue was purified and separated into Compound A101a (6 mg) and Compound A101b (9 mg) by Prep-HPLC(Method A).

Compound A101a (the earlier peak): ¹H NMR (400 MHz, CD₃OD) δ 7.94 (s, 1H), 7.87 (d, J=8.3 Hz, 1H), 7.77 (s, 1H), 7.54 (d, J=8.2 Hz, 1H), 5.62 (s, 1H), 4.89-4.65 (m, 3H), 3.91 (s, 2H), 3.90-3.87 (m, 1H), 3.73 (s, 3H), 3.19-3.12 (m, 2H), 3.02-2.96 (m, 1H), 2.90-2.82 (m, 1H), 2.48-2.41 (m, 1H), 1.60-1.52 (m, 2H), 1.46 (t, J=7.6 Hz, 3H), 1.40 (t, J=5.8 Hz, 6H), 0.73 (t, J=7.3 Hz, 3H) ppm. MS: M/e 504(M+1)⁺.

Compound A101b (the later peak): ¹H NMR (400 MHz, CD₃OD) δ 7.97 (s, 1H), 7.84 (d, J=8.4 Hz, 1H), 7.78 (s, 1H), 7.57 (d, J=8.3 Hz, 1H), 5.61 (s, 1H), 5.40-5.25 (m, 1H), 4.52-4.47 (m, 1H), 3.93 (s, 2H), 3.76 (d, J=5.9 Hz, 1H), 3.73 (s, 3H), 3.54 (d, J=11.3 Hz, 1H), 3.19-3.09 (m, 3H), 2.76 (d, J=8.3 Hz, 1H), 2.25 (d, J=11.5 Hz, 1H), 1.75-1.66 (m, 1H), 1.62-1.54 (m, 1H), 1.45 (t, J=7.6 Hz, 3H), 1.38 (d, J=6.5 Hz, 3H), 1.21 (d, J=6.5 Hz, 3H), 1.07 (t, J=7.4 Hz, 3H) ppm. MS: M/e 504(M+1)⁺.

Compound A102: 2-(8-((2S,5R)-2,5-diethyl-4-(3-methoxy-1-(2-methylbenzo[d]thiazol-6-yl)propyl)piperazin-1-yl)-5-methyl-6-oxo-5,6-dihydroimidazo[1,2-b]pyridazin-2-yl)acetonitrile

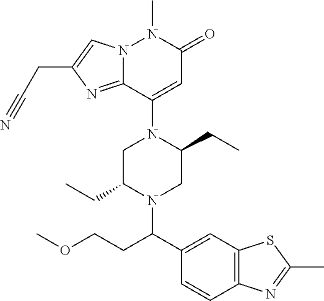 [SMILES: CC[C@H]1CN(C(CCOC)c2ccc3nc(C)sc3c2)[C@H](CC)CN1c1cc(=O)n(C)n2cc(CC#N)nc12]

### Step A: (E)-3-methoxy-1-(2-methylbenzo[d]thiazol-6-yl)prop-2-en-1-one

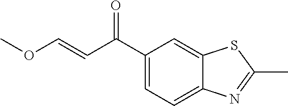

To a solution of 1-(2-methylbenzo[d]thiazol-6-yl)ethan-1-one (380 mg, 2 mol) in THF (20 mL) was added ethyl acetate (177 mg, 2.4 mol) and NaOMe (6 M, 0.34 ml, 2 mmol). The reaction mixture was stirred at 50° C. for 1 h. The mixture was cooled down to 0° C., A solution of CH₃I (459 mg, 3 mmol) in DMF (15 ml) was slowly added to the mixture and the reaction was stirred at rt for 1 h. The mixture was added H₂O (20 ml) and extracted with EA (20 mL×3), then concentrated by using a rotary evaporator to give a residue. The resulting residue was purified by flash column chromatography to give the titled compound (180 mg, 38.6%). MS: M/e 234 (M+1)⁺.

### Step B: 3-methoxy-1-(2-methylbenzo[d]thiazol-6-yl)propan-1-ol

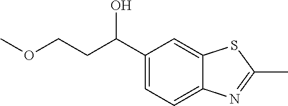

To a solution of (E)-3-methoxy-1-(2-methylbenzo[d]thiazol-6-yl)prop-2-en-1-one (260 mg, 1.1 mol) in MeOH was added NaBH₄ (128 mg, 3.4 mmol). The reaction mixture was stirred at RT for 15 mins. The mixture was added H₂O (20 ml) and extracted with DCM (20 mL×3). The organic phase was dried with Na₂SO₄ and concentrated by using a rotary evaporator to give a residue. The resulting residue was purified by flash column chromatography to give the titled compound (160 mg, 100%). MS: M/e 238 (M+1)⁺.

### Step C: 2-(8-((2S,5R)-2,5-diethyl-4-(3-methoxy-1-(2-methylbenzo[d]thiazol-6-yl)propyl)piperazin-1-yl)-5-methyl-6-oxo-5,6-dihydroimidazo[1,2-b]pyridazin-2-yl)acetonitrile

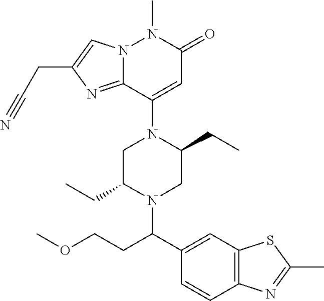 [SMILES: CC[C@H]1CN(C(CCOC)c2ccc3nc(C)sc3c2)[C@H](CC)CN1c1cc(=O)n(C)n2cc(CC#N)nc12]

To a solution of 2-(8-((2S,5R)-2,5-diethylpiperazin-1-yl)-5-methyl-6-oxo-5,6-dihydroimidazo[1,2-b]pyridazin-2-yl)acetonitrile (30 mg, 0.1 mmol), 3-methoxy-1-(2-methylbenzo[d]thiazol-6-yl)propan-1-ol (30 mg, 0.13 mmol) and (cyanomethyl)trimethylphosphonium iodide (120 mg, 0.5 mmol) in CH₃CN (3 mL) was added DIPEA (258 mg, 2 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified and separated into Compound A102a (1 mg) and Compound A102b (1 mg) by Prep-HPLC(Method B).

Compound A102a (the earlier peak): ¹H NMR (400 MHz, CD₃OD) δ 7.91-7.89 (m, 2H), 7.78 (s, 1H), 7.51-7.49 (m, 1H), 5.58 (s, 1H), 3.92 (s, 2H), 3.73 (s, 3H), 3.25 (m, 2H), 3.21-3.19 (m, 4H), 3.12 (m, 2H), 2.97 (m, 2H), 2.84 (s, 3H), 2.46 (m, 1H), 2.36 (m, 1H), 2.02 (m, 2H), 1.83 (m, 2H), 1.56 (m, 2H), 0.97 (t, J=7.0 Hz, 3H), 0.70-0.68 (m, 3H) ppm. MS: M/e 548 (M+1)⁺.

Compound A102b (the later peak): ¹H NMR (400 MHz, CD₃OD) δ 7.94 (s, 1H), 7.86 (d, J=8.0 Hz, 1H), 7.78 (s, 1H), 7.53 (d, J=7.5 Hz, 1H), 5.58 (s, 1H), 3.93 (s, 2H), 3.74-3.72 (m, 4H), 3.50 (d, J=14.3 Hz, 1H), 3.27 (m, 1H), 3.16 (m, 5H), 2.96 (d, J=7.2 Hz, 1H), 2.83 (s, 3H), 2.66 (m, 1H), 2.33 (m, 2H), 2.03 (m, 2H), 1.78 (m, 2H), 1.60 (m, 2H), 1.06-1.04 (m, 3H), 0.55-0.53 (m, 3H) ppm. MS: M/e 548 (M+1)⁺.

Compound A103: 2-(8-((2S,5R)-2-ethyl-4-(3-methoxy-1-(2-methylbenzo[d]thiazol-6-yl)propyl)-5-methylpiperazin-1-yl)-5-methyl-6-oxo-5,6-dihydroimidazo[1,2-b]pyridazin-2-yl)acetonitrile

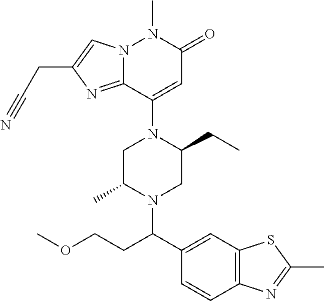 [SMILES: CC[C@H]1CN(C(CCOC)c2ccc3nc(C)sc3c2)[C@H](C)CN1c1cc(=O)n(C)n2cc(CC#N)nc12]

To a solution of 2-(8-((2S,5R)-2-ethyl-5-methylpiperazin-1-yl)-5-methyl-6-oxo-5,6-dihydroimidazo[1,2-b]pyridazin-2-yl)acetonitrile (30 mg, 0.1 mmol), 3-methoxy-1-(2-methylbenzo[d]thiazol-6-yl)propan-1-ol (50 mg, 0.2 mmol) and (cyanomethyl)trimethylphosphonium iodide (120 mg, 0.5 mmol) in CH₃CN (3 mL) was added DIPEA (258 mg, 2 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by Prep-HPLC(method B) to give the titled compound (2.8 mg). ¹H NMR (400 MHz, CD₃OD) δ 7.91 (d, J=3.8 Hz, 1H), 7.86 (t, J=9.1 Hz, 1H), 7.77 (s, 1H), 7.51 (d, J=8.4 Hz, 1H), 5.58 (s, 1H), 3.93 (d, J=3.0 Hz, 2H), 3.82 (m, 0.5H), 3.74 (d, J=10.4 Hz, 3H), 3.67 (m, 1H), 3.56 (m, 0.5H), 3.38 (m, 0.5H), 3.20 (m, 1H), 3.18 (s, 3H), 3.12 (m, 1H), 3.01 (m, 2H), 2.88 (s, 0.5H), 2.83 (s, 3H), 2.70 (d, J=9.1 Hz, 0.5H), 2.32 (m, 1.5H), 2.02 (m, 1H), 1.83 (m, 2H), 1.63 (s, 1H), 1.13 (d, J=6.4 Hz, 1.5H), 0.96 (d, J=7.0 Hz, 3H), 0.56 (t, J=7.1 Hz, 1.5H) ppm. MS: M/e 534 (M+1)⁺.

Compound A104: 2-(8-((2S,5R)-2,5-diethyl-4-(1-(2-methylthieno[2,3-b]pyridin-6-yl)ethyl)piperazin-1-yl)-5-methyl-6-oxo-5,6-dihydroimidazo[1,2-b]pyridazin-2-yl)acetonitrile

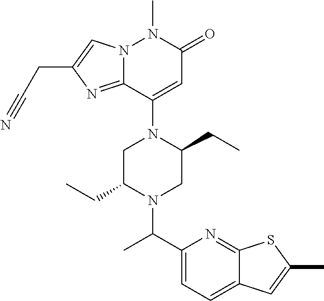

A solution of 2-(8-((2S,5R)-2,5-diethylpiperazin-1-yl)-5-methyl-6-oxo-5,6-dihydroimidazo[1,2-b]pyridazin-2-yl)acetonitrile (85 mg, 0.26 mol), 1-(2-methylthieno[2,3-b]pyridin-6-yl)ethan-1-ol (50 mg, 0.26 mmol), (cyanomethyl)trimethylphosphonium iodide (189 mg, 0.78 mol) and DIPEA (334 mg, 2.59 mol) in CH₃CN (2 ml) was stirred at 100° C. overnight. The reaction was diluted with EtOAc (10 ml), washed with brine (10 ml×2), dried and evaporated. The resulting residue was purified by flash column chromatography with 0-5% MeOH in DCM and then further purified by Prep-HPLC (Method A) to give the titled compound (53 mg). ¹H NMR (400 MHz, DMSO-d₆) δ 8.05 (d, J=8.2 Hz, 1H), 7.83 (d, J=3.8 Hz, 1H), 7.46 (dd, J=18.4, 8.3 Hz, 1H), 7.07 (s, 1H), 5.48 (s, 1H), 4.00 (d, J=3.6 Hz, 2H), 3.91 (t, J=6.6 Hz, 0.5H), 3.74 (t, J=6.5 Hz, 0.5H), 3.57 (s, 3H), 3.42-3.31 (m, 1.5H), 3.14 (d, J=11.2 Hz, 0.5H), 3.03 (d, J=9.2 Hz, 0.5H), 2.90-2.78 (m, 1H), 2.66-2.57 (m, 0.5H), 2.55 (s, 3H), 2.48 (s, 1H), 2.28 (d, J=9.8 Hz, 0.5H), 2.16 (d, J=11.2 Hz, 0.5H), 1.99-1.77 (m, 1H), 1.68-1.37 (m, 3H), 1.30 (dd, J=22.1, 6.6 Hz, 3H), 0.87 (dt, J=50.1, 7.2 Hz, 3H), 0.56 (dt, J=33.8, 7.1 Hz, 3H) ppm. MS: M/e 504 (M+1)⁺.

Compound A105: 2-(8-((2S,5R)-2,5-diethyl-4-(1-(2-fluorothieno[2,3-b]pyridin-6-yl)ethyl)piperazin-1-yl)-5-methyl-6-oxo-5,6-dihydroimidazo[1,2-b]pyridazin-2-yl)acetonitrile

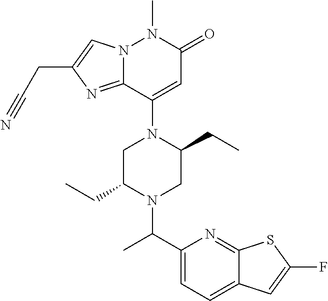 [SMILES: CC[C@H]1CN(C(C)c2ccc3cc(F)sc3n2)[C@H](CC)CN1c1cc(=O)n(C)n2cc(CC#N)nc12]

### Step A: 2-fluorothieno[2,3-b]pyridine

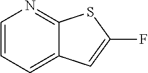

To a solution of thieno[2,3-b]pyridine (1.2 g, 8.89 mmol) in THF (15 ml) at −60° C. under N₂, was added n-BuLi (1.6 M, 6.94 ml, 11.11 mmol) dropwise and stirred for 60 min. NFSI (5.6 g, 17.78 mmol) was added to the above solution at −60° C. The reaction was warmed to RT naturally and stirred at RT overnight. The solution was quenched with brine (30 ml) and then extracted with EA (10 ml×2). The organic layer was dried and concentrated. The resulting residue was purified by flash column chromatography with 0-5% EA in PE to give the titled compound (410 mg, 30%). MS: M/e 154 (M+1)⁺.

### Step B: 2-fluorothieno[2,3-b]pyridine 7-oxide

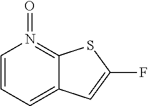 [SMILES: [O-][n+]1cccc2cc(F)sc21]

A solution of 2-fluorothieno[2,3-b]pyridine (520 mg, 3.40 mmol) and mCPBA (1.18 g, 6.82 mmol) in DCM (10 ml) was stirred at RT overnight. The reaction was washed with brine (10 ml×2), dried over Na₂SO₄ and concentrated to dryness. The resulting residue was purified by flash column chromatography with 0-10% MeOH in DCM to give the titled compound (500 mg, 87%). MS: M/e 170 (M+1)⁺.

### Step C: 2-fluorothieno[2,3-b]pyridine-6-carbonitrile

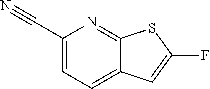 [SMILES: N#Cc1ccc2cc(F)sc2n1]

A solution of 2-fluorothieno[2,3-b]pyridine 7-oxide (500 mg, 2.96 mmol), TMSCN (879 mg, 8.88 mmol), dimethylcarbamic chloride (959 mg, 8.88 mmol) and TEA (896 mg, 8.88 mmol) in MeCN (10 ml) was stirred at 70° C. for 6.5 hrs. The solution was diluted with EtOAc (15 ml), washed with brine (10 ml×2), dried over Na₂SO₄ and concentrated to dryness. The resulting residue was purified by flash column chromatography with 5-20% EA in PE to give the titled compound (410 mg, 78%). MS: M/e 179 (M+1)⁺.

### Step D: 1-(2-fluorothieno[2,3-b]pyridin-6-yl)ethan-1-one

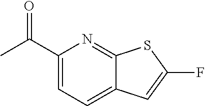

A solution of 2-fluorothieno[2,3-b]pyridine-6-carbonitrile (410 mg, 2.30 mmol), MeMgBr (1M, 3.46 ml, 3.46 mmol) and CuBr (33 mg, 0.23 mmol) in THF (4 ml) under N₂ was stirred at 60° C. overnight. The solution was diluted with EtOAc (10 ml), washed with brine (10 ml×2), dried over Na₂SO₄ and concentrated to dryness. The resulting residue was purified by flash column chromatography with 0-10% EA in PE to give the titled compound (170 mg, 38%). MS: M/e 196 (M+1)⁺.

### Step E: 1-(2-fluorothieno[2,3-b]pyridin-6-yl)ethan-1-ol

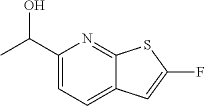

A solution of 1-(2-fluorothieno[2,3-b]pyridin-6-yl)ethan-1-one (170 mg, 0.87 mmol) and NaBH₄ (33.1 mg, 0.87 mmol) in MeOH (2 ml) was stirred at 0° C. for 40 min. The solution was diluted with EtOAc (10 ml), washed with brine (10 ml×2), dried over Na₂SO₄ and concentrated to dryness to give the titled compound (170 mg, 99%), which was used directly for the next step without further purification. MS: M/e 198 (M+1)⁺.

### Step F: 2-(8-((2S,5R)-2,5-diethyl-4-(1-(2-fluorothieno[2,3-b]pyridin-6-yl)ethyl)piperazin-1-yl)-5-methyl-6-oxo-5,6-dihydroimidazo[1,2-b]pyridazin-2-yl)acetonitrile

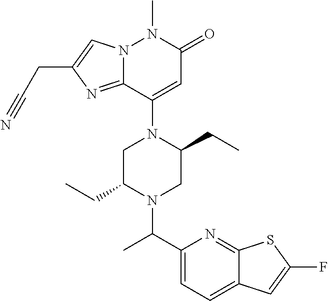 [SMILES: CC[C@H]1CN(C(C)c2ccc3cc(F)sc3n2)[C@H](CC)CN1c1cc(=O)n(C)n2cc(CC#N)nc12]

A solution of 2-(8-((2S,5R)-2,5-diethylpiperazin-1-yl)-5-methyl-6-oxo-5,6-dihydroimidazo[1,2-b]pyridazin-2-yl)acetonitrile (83.2 mg, 0.25 mmol), 1-(2-fluorothieno[2,3-b]pyridin-6-yl)ethan-1-ol (50 mg, 0.25 mmol), (cyanomethyl)trimethylphosphonium iodide (185 mg, 0.76 mmol) and DIPEA (327 mg, 2.53 mmol) in CH₃CN (2 ml) was stirred at 100° C. overnight. The reaction was diluted with EtOAc (10 ml) and washed with brine (10 ml). The organic layer was concentrated under reduced pressure. The resulting residue was purified by flash column chromatography and further separated into Compound A105a (20 mg) and Compound A105b (20 mg) by Prep-HPLC (Method A).

Compound A105a (the earlier peak):¹H NMR (400 MHz, DMSO-d₆) δ 8.11 (d, J=8.3 Hz, 1H), 7.83 (s, 1H), 7.59 (d, J=8.3 Hz, 1H), 7.07 (d, J=4.1 Hz, 1H), 5.47 (s, 1H), 4.00 (s, 2H), 3.75 (q, J=6.5 Hz, 1H), 3.58 (s, 3H), 3.44-3.29 (m, 2H), 3.02 (d, J=9.7 Hz, 1H), 2.60 (d, J=9.3 Hz, 1H), 2.48 (s, 1H), 2.16 (d, J=11.6 Hz, 1H), 1.91-1.76 (m, 1H), 1.61-1.33 (m, 3H), 1.27 (d, J=6.6 Hz, 3H), 0.93 (t, J=7.2 Hz, 3H), 0.53 (t, J=7.2 Hz, 3H) ppm. MS: M/e 508 (M+1)⁺.

Compound A105b (the later peak):¹H NMR (400 MHz MHz, DMSO-d₆) δ 8.11 (d, J=8.2 Hz, 1H), 7.82 (s, 1H), 7.55 (d, J=8.2 Hz, 1H), 7.08 (d, J=4.1 Hz, 1H), 5.48 (s, 1H), 3.99 (s, 2H), 3.93 (q, J=6.6 Hz, 1H), 3.57 (s, 3H), 3.30-3.26 (m, 1H), 3.15 (d, J=12.0 Hz, 1H), 2.84 (s, 2H), 2.49 (s, 1H), 2.30 (d, J=7.4 Hz, 1H), 1.99-1.85 (m, 1H), 1.69-1.56 (m, 1H), 1.54-1.38 (m, 2H), 1.33 (d, J=6.6 Hz, 3H), 0.81 (t, J=7.3 Hz, 3H), 0.62 (t, J=7.2 Hz, 3H) ppm. MS: M/e 508 (M+1)⁺.

Compound A106: 2-(8-((2S,5R)-5-ethyl-4-(1-(2-fluorothieno[2,3-b]pyridin-6-yl)ethyl)-2-methylpiperazin-1-yl)-5-methyl-6-oxo-5,6-dihydroimidazo[1,2-b]pyridazin-2-yl)acetonitrile

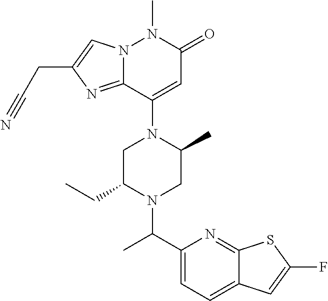 [SMILES: CC[C@@H]1CN(c2cc(=O)n(C)n3cc(CC#N)nc23)[C@@H](C)CN1C(C)c1ccc2cc(F)sc2n1]

A solution of 2-(8-((2S,5R)-5-ethyl-2-methylpiperazin-1-yl)-5-methyl-6-oxo-5,6-dihydroimidazo[1,2-b]pyridazin-2-yl)acetonitrile (80 mg, 0.25 mmol), 1-(2-fluorothieno[2,3-b]pyridin-6-yl)ethan-1-ol (50 mg, 0.25 mmol), (cyanomethyl)trimethylphosphonium iodide (185 mg, 0.76 mmol) and DIPEA (327 mg, 2.53 mmol) in CH₃CN (2 ml) was stirred at 100° C. overnight. The reaction was diluted with EtOAc (10 ml) and washed with brine (10 ml). The organic layer was concentrated under reduced pressure. The resulting residue was purified to give the titled compound (68 mg) by Prep-HPLC (Method A). ¹H NMR (400 MHz, DMSO-d₆) δ 8.11 (dd, J=8.3, 3.2 Hz, 1H), 7.83 (d, J=4.6 Hz, 1H), 7.59 (dd, J=23.4, 8.3 Hz, 1H), 7.07 (t, J=4.0 Hz, 1H), 5.50 (d, J=2.8 Hz, 1H), 5.40-4.10 (m, 2H), 4.00 (d, J=7.4 Hz, 2H), 3.93 (q, J=6.6 Hz, 0.5H), 3.77 (q, J=6.6 Hz, 0.5H), 3.58 (s, 3H), 3.39 (d, J=10.5 Hz, 0.5H), 3.18 (d, J=9.9 Hz, 0.5H), 3.02 (d, J=9.5 Hz, 0.5H), 2.90 (dd, J=11.8, 4.1 Hz, 0.5H), 2.73-2.62 (m, 1H), 2.32 (s, 0.5H), 2.09 (d, J=9.9 Hz, 0.5H), 1.62-1.36 (m, 2H), 1.31 (dd, J=14.7, 6.6 Hz, 3H), 1.21 (d, J=6.4 Hz, 1.5H), 1.10 (d, J=6.4 Hz, 1.5H), 0.97 (t, J=7.3 Hz, 1.5H), 0.67 (t, J=7.3 Hz, 1.5H) ppm. MS: M/e 494 (M+1)⁺.

Compound A107: 2-(8-((2S,5R)-2,5-diethyl-4-(1-(2-ethylthiazolo[5,4-b]pyridin-5-yl)ethyl)piperazin-1-yl)-5-methyl-6-oxo-5,6-dihydroimidazo[1,2-b]pyridazin-2-yl)acetonitrile

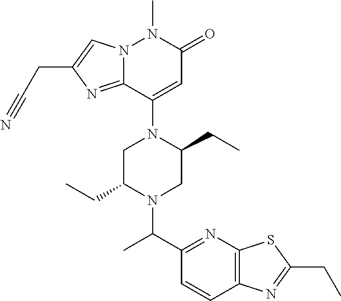 [SMILES: CCc1nc2ccc(C(C)N3C[C@H](CC)N(c4cc(=O)n(C)n5cc(CC#N)nc45)C[C@H]3CC)nc2s1]

### Step A: N-(2,6-dichloropyridin-3-yl)propionamide

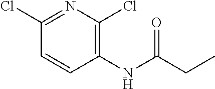

To a solution of 2,6-dichloropyridin-3-amine (1 g, 6.1 mmol) in DMF (15 mL) were added HATU (3.5 g, 9.2 mmol), DIPEA (2.4 g, 18.5 mol) and propionic acid (0.9 g, 12.2 mol). The mixture was stirred at 50° C. for overnight. The reaction was diluted with EA and washed with water. The organic layer was separated, dried by Na₂SO₄, filtered, and concentrated to dryness. The resulting residue was purified by flash column chromatography to give the titled compound (1 g, 74%). MS: M/e 219 (M+1)⁺.

### Step B: 5-chloro-2-ethylthiazolo[5,4-b]pyridine

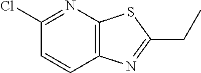

To a solution of N-(2,6-dichloropyridin-3-yl)propionamide (1 g, 4.5 mmol) in toluene (15 mL) was added lawesson reagent (0.93 g, 2.3 mmol) and the resulting mixture was stirred at 100° C. for 1 hours. The reaction mixture was concentrated and purified by flash column chromatography to give the titled compound (0.5 g, 45%). MS: M/e 199 (M+1)⁺.

### Step C: 1-(2-ethylthiazolo[5,4-b]pyridin-5-yl)ethan-1-one

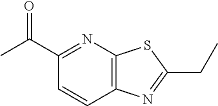

A mixture of 5-chloro-2-ethylthiazolo[5,4-b]pyridine (0.5 g, 5.7 mmol), tributyl(1-ethoxyvinyl)stannane (3.1 g, 8.5 mmol) and Pd(PPh₃)₂Cl₂ (399 mg, 0.57 mmol) in toluene (20 mL) was stirred at 100° C. under N₂ for 16 hours. The solution was diluted with ethyl acetate and washed with water. The combined organic layers were washed with brine, dried over Na₂SO₄ and the resulting residue was purified by flash column chromatography to give the titled compound (0.2 g, 38%). MS: M/e 207(M+1)⁺

### Step D: 1-(2-ethylthiazolo[5,4-b]pyridin-5-yl)ethan-1-ol

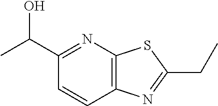

To a solution of 1-(2-ethylthiazolo[5,4-b]pyridin-5-yl)ethan-1-one (0.2 g, 0.98 mmol) in MeOH (5 mL) was added NaBH₄ (37 mg, 0.98 mmol) at room temperature and the resulting mixture was stirred at room temperature for 5 min. The reaction mixture was diluted with DCM and washed with water, dried over Na₂SO₄, concentrated and purified by flash column chromatography to give the titled compound (170 mg, 85%). MS: M/e 209 (M+1)⁺.

### Step E: 2-(8-((2S,5R)-2,5-diethyl-4-(1-(2-ethylthiazolo[5,4-b]pyridin-5-yl)ethyl)piperazin-1-yl)-5-methyl-6-oxo-5,6-dihydroimidazo[1,2-b]pyridazin-2-yl)acetonitrile

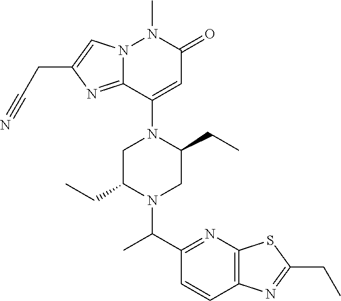 [SMILES: CCc1nc2ccc(C(C)N3C[C@H](CC)N(c4cc(=O)n(C)n5cc(CC#N)nc45)C[C@H]3CC)nc2s1]

To a solution of 2-(8-((2S,5R)-2,5-diethylpiperazin-1-yl)-5-methyl-6-oxo-5,6-dihydroimidazo[1,2-b]pyridazin-2-yl)acetonitrile (50 mg, 0.15 mmol) in CH₃CN (3 mL) and was added 1-(2-ethylthiazolo[5,4-b]pyridin-5-yl)ethan-1-ol (64 mg, 0.3 mmol), (cyanomethyl)trimethylphosphonium iodide (111 mg, 0.45 mmol) and DIPEA (196 mg, 1.5 mmol). The resulting mixture was stirred at 105° C. overnight. The reaction solvent was removed under reduce pressure. The resulting residue was purified by flash column chromatography to give the titled compound (crude), which was further separated into Compound A107a (17 mg) and Compound A107b (16 mg) by Prep-HPLC (Method A).

Compound A107a (the earlier peak): ¹H NMR (400 MHz, CD₃OD) δ 8.24 (d, J=8.5 Hz, 1H), 7.83-7.71 (m, 2H), 5.60 (s, 1H), −3.93 (s, 2H), 3.91-3.83 (m, 1H), 3.73 (s, 3H), 3.55 (d, J=13.4 Hz, 1H), 3.33-3.30 (m, 2H), 3.18 (q, J=7.6 Hz, 3H), 2.80 (s, 1H), 2.31 (s, 1H), 2.08-1.52 (m, 4H), 1.47 (t, J=7.6 Hz, 3H), 1.41 (d, J=6.4 Hz, 3H), 1.05 (t, J=7.2 Hz, 3H), 0.63 (t, J=7.2 Hz, 3H) ppm. MS: M/e 519 (M+1)⁺.

Compound A107b (the later peak): ¹H NMR (400 MHz, CD₃OD) δ 8.24 (d, J=8.5 Hz, 1H), 7.78 (s, 1H), 7.71 (d, J=8.5 Hz, 1H), 5.60 (s, 1H), 4.07 (q, J=6.5 Hz, 1H), 3.92 (s, 2H), 3.73 (s, 3H), 3.34 (d, J=11.1 Hz, 1H), 3.33-3.30 (m, 2H), 3.18 (q, J=7.6 Hz, 2H), 3.07-2.93 (m, 2H), 2.40 (d, J=8.8 Hz, 1H), 2.15-1.52 (m, 4H), 1.50-1.38 (m, 6H), 0.94 (t, J=7.4 Hz, 3H), 0.71 (t, J=7.4 Hz, 3H) ppm. MS: M/e 519 (M+1)⁺.

Compound A108: 2-(8-((2R,5R)-5-ethyl-2-(methoxymethyl)-4-(1-(2-methylthiazolo[5,4-b]pyridin-5-yl)ethyl)piperazin-1-yl)-5-methyl-6-oxo-5,6-dihydroimidazo[1,2-b]pyridazin-2-yl)acetonitrile

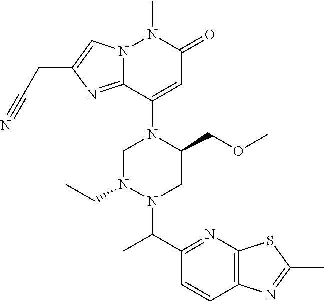

### Step A: ethyl 8-((2R,5R)-4-(tert-butoxycarbonyl)-5-ethyl-2-(hydroxymethyl)piperazin-1-yl)-6-chloroimidazo[1,2-b]pyridazine-2-carboxylate

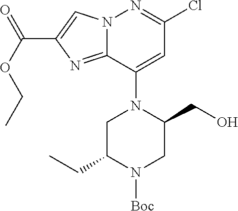 [SMILES: CCOC(=O)c1cn2nc(Cl)cc(N3C[C@@H](CC)N(C(=O)OC(C)(C)C)C[C@@H]3CO)c2n1]

To a mixture of the ethyl 8-bromo-6-chloroimidazo[1,2-b]pyridazine-2-carboxylate (6.1 g, 20 mmol) and tert-butyl (2R,5R)-2-ethyl-5-(hydroxymethyl)piperazine-1-carboxylate (4.88 g, 20 mmol) in CH₃CN (60 mL) was added DIPEA (4.75 g, 40 mmol). The reaction was stirred at 100° C. overnight in a sealed tube. The reaction mixture was poured into H₂O (200 mL), extracted with EtOAc (100 mL×2). The combined organic layers were washed with brine, dried over Na₂SO₄, filtered and concentrated. The resulting residue was purified by flash column chromatography to give the titled compound (5 g, 53%). MS: M/e 468 (M+1)*.

### Step B: ethyl 8-((2R,5R)-4-(tert-butoxycarbonyl)-5-ethyl-2-(methoxymethyl)piperazin-1-yl)-6-chloroimidazo[1,2-b]pyridazine-2-carboxylate

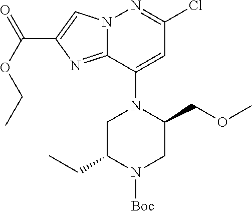 [SMILES: CCOC(=O)c1cn2nc(Cl)cc(N3C[C@@H](CC)N(C(=O)OC(C)(C)C)C[C@@H]3COC)c2n1]

To a solution of ethyl 8-((2R,5R)-4-(tert-butoxycarbonyl)-5-ethyl-2-(hydroxymethyl)piperazin-1-yl)-6-chloroimidazo[1,2-b]pyridazine-2-carboxylate (2.34 g, 5 mmol) in THF (40 mL) was added 60% NaH in oil (600 mg, 15 mmol) at 0° C. in some portions. After 30 min, CH₃I (3.55 g, 25 mmol) was added and the reaction mixture was stirred at RT for 3 hours. The reaction was warmed to 50° C. overnight. The mixture was cooled to room temperature, diluted with citric acid solution/water (10 mL/40 mL), extracted with EtOAc (80 mL×3), washed with brine, dried over Na₂SO₄, filtered, and concentrated to give the titled compound (2.4 g, crude). MS: M/e 482 (M+1)⁺.

### Step C: 8-((2R,5R)-4-(tert-butoxycarbonyl)-5-ethyl-2-(methoxymethyl)piperazin-1-yl)-6-hydroxyimidazo[1,2-b]pyridazine-2-carboxylic acid

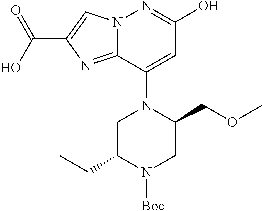 [SMILES: CC[C@@H]1CN(c2cc(O)nn3cc(C(=O)O)nc23)[C@@H](COC)CN1C(=O)OC(C)(C)C]

To a stirred solution of ethyl 8-((2R,5R)-4-(tert-butoxycarbonyl)-5-ethyl-2-(methoxymethyl)piperazin-1-yl)-6-chloroimidazo[1,2-b]pyridazine-2-carboxylate (2.4 g, 5 mmol) in dioxane (20 mL) was added aq.KOH (1.68 g, 30 mmol, in 10 mL H₂O). Then Pd₂(dba)₃ (458 mg, 0.5 mmol) and t-BuXPhos (424 mg, 1 mmol) were added under N₂. After the addition, the reaction mixture was stirred at 100° C. for 3 hours. The reaction mixture was poured into H₂O (50 mL) and extracted with dcm (60 mL×2). The aqueous layer was collected, acidified to pH=3-4 with saturated citric acid. A precipitate was formed, filtered and dried to give the titled compound (1.85 g, 85% for two steps). MS: M/e 436 (M+1)⁺.

### Step D: tert-butyl (2R,5R)-2-ethyl-4-(6-hydroxy-2-(hydroxymethyl)imidazo[1,2-b]pyridazin-8-yl)-5-(methoxymethyl)piperazine-1-carboxylate

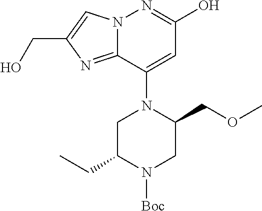 [SMILES: CC[C@@H]1CN(c2cc(O)nn3cc(CO)nc23)[C@@H](COC)CN1C(=O)OC(C)(C)C]

To a mixture of 8-((2R,5R)-4-(tert-butoxycarbonyl)-5-ethyl-2-(methoxymethyl)piperazin-1-yl)-6-hydroxyimidazo[1,2-b]pyridazine-2-carboxylic acid (1.85 g, 4.25 mmol) in THF (20 mL) was added CDI (1.03 g, 6.37 mmol). The reaction mixture was stirred at room temperature for 2 hours.

To another bottle was charged with NaBH₄ (484 mg, 12.75 mmol) in THF/H₂O (20 mL/16 mL). Then the first mixture was added drop wise to the NaBH₄ solution at 0° C. After addition, the reaction was stirred at room temperature for one hour. The reaction was quenched with saturated citric acid solution, extracted with EA (60 mL×3), washed with brine, dried over Na₂SO₄, filtered, and concentrated to give the titled compound (1.76 g, crude, 98%). MS: M/e 422 (M+1)⁺.

### Step E: tert-butyl (2R,5R)-2-ethyl-4-(2-(hydroxymethyl)-5-methyl-6-oxo-5,6-dihydroimidazo[1,2-b]pyridazin-8-yl)-5-(methoxymethyl)piperazine-1-carboxylate

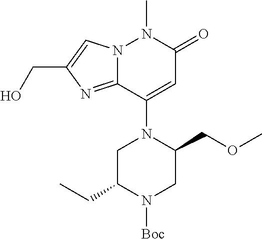 [SMILES: CC[C@@H]1CN(c2cc(=O)n(C)n3cc(CO)nc23)[C@@H](COC)CN1C(=O)OC(C)(C)C]

To a solution of tert-butyl (2R,5R)-2-ethyl-4-(6-hydroxy-2-(hydroxymethyl)imidazo[1,2-b]pyridazin-8-yl)-5-(methoxymethyl)piperazine-1-carboxylate (1.76 g, 4.18 mmol) in CH₃CN (18 mL) was added HMDS (805 mg, 5 mmol). The reaction was heated at 80° C. for 30 min and chloro(chloromethyl)dimethylsilane (715 mg, 5 mmol) was added. The resulted mixture was heated at 80° C. for 16 hours. The mixture was concentrated to dryness under reduced pressure. The residue was dissolved in DMSO (25 mL) and a solution of KF (1.7 g, 29.26 mmol) was added. The reaction was heated at 100° C. for one hour. Most of DMSO was concentrated off. The residue was diluted with water, extracted with EA (60 mL×2), washed with brine, dried over Na₂SO₄, filtered, and concentrated to dryness. The resulting residue was purified by flash column chromatography to give the titled compound (1.16 g, 63% for two steps). MS: M/e 436 (M+1)⁺.

### Step F: tert-butyl (2R,5R)-4-(2-(chloromethyl)-5-methyl-6-oxo-5,6-dihydroimidazo[1,2-b]pyridazin-8-yl)-2-ethyl-5-(methoxymethyl)piperazine-1-carboxylate

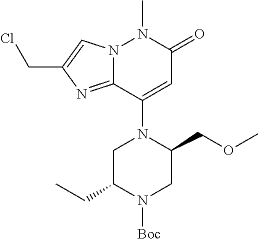 [SMILES: CC[C@@H]1CN(c2cc(=O)n(C)n3cc(CCl)nc23)[C@@H](COC)CN1C(=O)OC(C)(C)C]

To a stirred solution of tert-butyl (2R,5R)-2-ethyl-4-(2-(hydroxymethyl)-5-methyl-6-oxo-5,6-dihydroimidazo[1,2-b]pyridazin-8-yl)-5-(methoxymethyl)piperazine-1-carboxylate (1.16 g, 2.66 mmol) in CH₂Cl₂ (10 mL) was added SOCl₂ (350 mg, 2.93 mmol). After the addition, the reaction was stirred for 20 min. The reaction mixture was washed with aq.NaHCO₃, extracted with DCM (60 mL×2), washed with brine, dried over Na₂SO₄, concentrated to give the titled compound (1.2 g, crude). MS: M/e 454 (M+1)⁺.

### Step G: tert-butyl (2R,5R)-4-(2-(cyanomethyl)-5-methyl-6-oxo-5,6-dihydroimidazo[1,2-b]pyridazin-8-yl)-2-ethyl-5-(methoxymethyl)piperazine-1-carboxylate

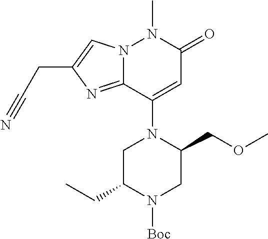 [SMILES: CC[C@@H]1CN(c2cc(=O)n(C)n3cc(CC#N)nc23)[C@@H](COC)CN1C(=O)OC(C)(C)C]

To a stirred solutiong of tert-butyl (2R,5R)-4-(2-(chloromethyl)-5-methyl-6-oxo-5,6-dihydroimidazo[1,2-b]pyridazin-8-yl)-2-ethyl-5-(methoxymethyl)piperazine-1-carboxylate (1.2 g, 2.66 mmol) in CH₂Cl₂ (10 mL) was added TMSCN (0.53 g, 5.32 mmol), followed by HMDS (1.0 M, 5.3 mL, 5.3 mmol). After the addition, the reaction mixture was stirred overnight. The reaction mixture was concentrated and purified by flash column chromatography to give the titled compound (700 mg, 59% for two steps). MS: M/e 445 (M+1)⁺.

### Step H: 2-(8-((2R,5R)-5-ethyl-2-(methoxymethyl)piperazin-1-yl)-5-methyl-6-oxo-5,6-dihydroimidazo[1,2-b]pyridazin-2-yl)acetonitrile

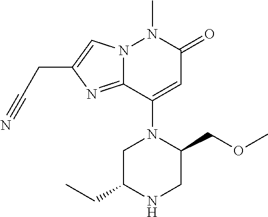 [SMILES: CC[C@@H]1CN(c2cc(=O)n(C)n3cc(CC#N)nc23)[C@@H](COC)CN1]

To a stirred solution of tert-butyl (2R,5R)-4-(2-(cyanomethyl)-5-methyl-6-oxo-5,6-dihydroimidazo[1,2-b]pyridazin-8-yl)-2-ethyl-5-(methoxymethyl)piperazine-1-carboxylate (0.7 g, 1.58 mmol) in CH₂Cl₂ (10 mL) was added TMSOTf (0.7 g, 3.15 mmol). After the addition, the reaction was stirred for 3 hours. The reaction mixture was quenched with aq.NaHCO₃, then extracted with CH₂Cl₂/IPA (3/1, 40 mL×6). The combined organic layers were dried over Na₂SO₄, concentrated to give the titled compound (0.5 g, 92%). MS: M/e 345 (M+1)⁺

### Step I: 2-(8-((2R,5R)-5-ethyl-2-(methoxymethyl)-4-(1-(2-methylthiazolo[5,4-b]pyridin-5-yl)ethyl)piperazin-1-yl)-5-methyl-6-oxo-5,6-dihydroimidazo[1,2-b]pyridazin-2-yl)acetonitrile

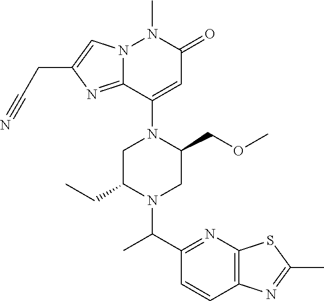 [SMILES: CC[C@@H]1CN(c2cc(=O)n(C)n3cc(CC#N)nc23)[C@@H](COC)CN1C(C)c1ccc2nc(C)sc2n1]

A mixture of 2-(8-((2R,5R)-5-ethyl-2-(methoxymethyl)piperazin-1-yl)-5-methyl-6-oxo-5,6-dihydroimidazo[1,2-b]pyridazin-2-yl)acetonitrile (35 mg, 0.1 mmol), 1-(2-methylthiazolo[5,4-b]pyridin-5-yl)ethan-1-ol (40 mg, 0.2 mmol), (cyanomethyl)trimethylphosphonium iodide (74 mg, 0.3 mmol) and DIPEA (129 mg, 1 mmol) in CH₃CN (2 mL) was stirred at 100° C. overnight in a sealed tube. The reaction mixture was diluted with EtOAc (10 mL), washed with brine, dried over Na₂SO₄, concentrated. The resulting residue was purified by Prep-TLC (EA) and further separated into Compound A108a (9 mg) and Compound A108b (9 mg) by Prep-HPLC (Method A).

Compound A108a (the earlier peak): ¹H NMR (400 MHz, CD₃OD) δ 8.20 (d, J=8.0 Hz, 1H), 7.77 (s, 1H), 7.71 (d, J=8.0 Hz, 1H), 5.68 (s, 1H), 3.92 (s, 2H), 3.89-3.82 (m, 1H), 3.72 (s, 3H), 3.68-3.56 (m, 2H), 3.53-3.44 (m, 1H), 3.35-3.31 (m, 2H), 3.23-3.17 (m, 1H), 3.13 (s, 3H), 2.84 (s, 3H), 2.83-2.77 (m, 1H), 2.40-2.30 (m, 1H), 1.80-1.65 (m, 1H), 1.60-1.48 (m, 1H), 1.38 (d, J=6.8 Hz, 3H), 1.01 (t, J=6.8 Hz, 3H) ppm. MS: M/e 521 (M+1)⁺.

Compound A108b (the later peak):¹H NMR (400 MHz, CD₃OD) δ 8.21 (d, J=8.0 Hz, 1H), 7.77 (s, 1H), 7.68 (d, J=8.4 Hz, 1H), 5.67 (s, 1H), 4.08-3.99 (m, 1H), 3.91 (s, 2H), 3.89-3.82 (m, 1H), 3.79-3.73 (m, 1H), 3.72 (s, 3H), 3.35-3.31 (m, 5H), 3.29-3.21 (m, 1H), 3.15-3.08 (m, 1H), 3.03-2.94 (m, 1H), 2.84 (s, 3H), 2.44-2.32 (m, 1H), 1.65-1.55 (m, 2H), 1.40 (d, J=6.8 Hz, 3H), 0.68 (t, J=6.8 Hz, 3H) ppm. MS: M/e 521 (M+1)⁺.

Compound A109: 2-(8-((2S,5R)-4-(1-(2-cyclopropylthiazolo[5,4-b]pyridin-5-yl)ethyl)-2,5-diethylpiperazin-1-yl)-5-methyl-6-oxo-5,6-dihydroimidazo[1,2-b]pyridazin-2-yl)acetonitrile

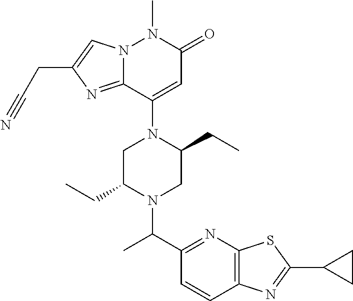 [SMILES: CC[C@H]1CN(C(C)c2ccc3nc(C4CC4)sc3n2)[C@H](CC)CN1c1cc(=O)n(C)n2cc(CC#N)nc12]

### Step A: N-(2,6-dichloropyridin-3-yl)cyclopropanecarboxamide

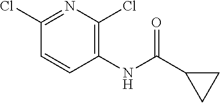 [SMILES: O=C(Nc1ccc(Cl)nc1Cl)C1CC1]

To a solution of 2,6-dichloropyridin-3-amine (1 g, 6.1 mmol) in DMF (15 mL) were added HATU (3.5 g, 9.2 mmol), DIPEA (2.4 g, 18.5 mol) and cyclopropanecarboxylic acid (1.05 g, 12.2 mol). The mixture was stirred at 50° C. for overnight. The reaction was diluted with EA and washed with water. The organic layer was separated, dried by Na₂SO₄, filtered, and concentrated to dryness. The resulting residue was purified by flash column chromatography to give the titled compound (1.2 g, crude). MS: M/e 231 (M+1)⁺.

### Step B: 5-chloro-2-cyclopropylthiazolo[5,4-b]pyridine

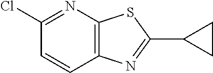 [SMILES: Clc1ccc2nc(C3CC3)sc2n1]

To a solution of N-(2,6-dichloropyridin-3-yl)cyclopropanecarboxamide (1.2 g, 5.1 mmol) in toluene (15 mL) was added lawesson reagent (1.05 g, 2.5 mmol) and the resulting mixture was stirred at 100° C. for 1 hours. The reaction mixture was concentrated and purified by flash column chromatography to give the titled compound (0.7 g). MS: M/e 211 (M+1)⁺.

### Step C: 1-(2-cyclopropylthiazolo[54-b]pyridin-5-yl)ethan-1-one

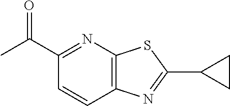

A mixture of 5-chloro-2-cyclopropylthiazolo[5,4-b]pyridine (0.7 g, 3.3 mmol), tributyl(1-ethoxyvinyl)stannane (1.8 g, 5 mmol) and Pd(PPh₃)₂Cl₂ (231 mg, 0.57 mmol) in toluene (20 mL) was stirred at 100° C. under N₂ for 16 hours. The solution was diluted with ethyl acetate and washed with water. The combined organic layers were washed with brine, dried over Na₂SO₄ and the resulting residue was purified by flash column chromatography to give the titled compound (0.17 g). MS: M/e 218(M+1)⁺

### Step D: 1-(2-cyclopropylthiazolo[5,4-b]pyridin-5-yl)ethan-1-ol

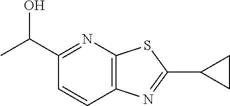

To a solution of 1-(2-cyclopropylthiazolo[5,4-b]pyridin-5-yl)ethan-1-one (0.2 g, 0.91 mmol) in MeOH (5 mL) was added NaBH₄ (34 mg, 0.91 mmol) at room temperature and the resulting mixture was stirred at room temperature for 5 min. The reaction mixture was diluted with DCM and washed with water, dried over Na₂SO₄, concentrated to give the titled compound (160 mg, 85%). MS: M/e 220 (M+1)⁺.

### Step E: 2-(8-((2S,5R)-4-(1-(2-cyclopropylthiazolo[5,4-b]pyridin-5-yl)ethyl)-2,5-diethylpiperazin-1-yl)-5-methyl-6-oxo-5,6-dihydroimidazo[1,2-b]pyridazin-2-yl)acetonitrile

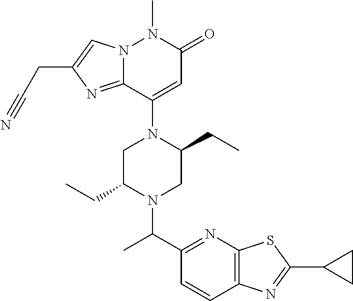 [SMILES: CC[C@H]1CN(C(C)c2ccc3nc(C4CC4)sc3n2)[C@H](CC)CN1c1cc(=O)n(C)n2cc(CC#N)nc12]

To a solution of 2-(8-((2S,5R)-2,5-diethylpiperazin-1-yl)-5-methyl-6-oxo-5,6-dihydroimidazo[1,2-b]pyridazin-2-yl)acetonitrile (50 mg, 0.15 mmol) in CH₃CN (3 mL) and was added 1-(2-cyclopropylthiazolo[5,4-b]pyridin-5-yl)ethan-1-ol (67 mg, 0.3 mmol), (cyanomethyl)trimethylphosphonium iodide (111 mg, 0.45 mmol) and DIPEA (196 mg, 1.5 mmol). The resulting mixture was stirred at 105° C. overnight. The reaction solvent was removed under reduce pressure. The resulting residue was purified by flash column chromatography to give the titled compound (crude), which was further separated into Compound A109a (13 mg) and Compound A109b (19 mg) by Prep-HPLC (Method A).

Compound A109a (the earlier peak): ¹H NMR (400 MHz, CD₃OD) δ 8.14 (d, J=8.5 Hz, 1H), 7.78 (s, 1H), 7.72 (d, J=8.5 Hz, 1H), 5.59 (s, 1H), 3.93 (s, 2H), 3.87 (q, J=6.5 Hz, 1H), 3.73 (s, 3H), 3.53 (d, J=12.1 Hz, 1H), 3.37-3.30 (m, 2H), 3.17 (d, J=9.8 Hz, 1H), 2.78 (dd, J=12.2, 3.6 Hz, 1H), 2.52-2.41 (m, 1H), 2.27 (d, J=12.1 Hz, 1H), 2.07-1.47 (m, 4H), 1.39 (d, J=6.7 Hz, 3H), 1.34-1.15 (m, 4H), 1.04 (t, J=7.3 Hz, 3H), 0.62 (t, J=7.4 Hz, 3H) ppm. MS: M/e 531 (M+1)⁺.

Compound A109b (the later peak): ¹H NMR (400 MHz, CD₃OD) δ 8.15 (d, J=8.4 Hz, 1H), 7.78 (s, 1H), 7.66 (d, J=8.5 Hz, 1H), 5.60 (s, 1H), 4.04 (q, J=6.7 Hz, 1H), 3.92 (s, 2H), 3.73 (s, 3H), 3.39-3.32 (m, 2H), 3.05-2.91 (m, 2H), 2.53-2.45 (m, 1H), 2.39 (d, J=9.1 Hz, 1H), 2.16-1.49 (m, 5H), 1.42 (d, J=6.6 Hz, 3H), 1.34-1.18 (m, 4H), 0.94 (t, J=7.4 Hz, 3H), 0.71 (t, J=7.3 Hz, 3H) ppm. MS: M/e 531 (M+1)⁺.

Compound A110: 2-(8-((2S,5R)-4-(1-(2-(cyclopropylmethyl)thiazolo[5,4-b]pyridin-5-yl)ethyl)-2,5-diethylpiperazin-1-yl)-5-methyl-6-oxo-5,6-dihydroimidazo[1,2-b]pyridazin-2-yl)acetonitrile

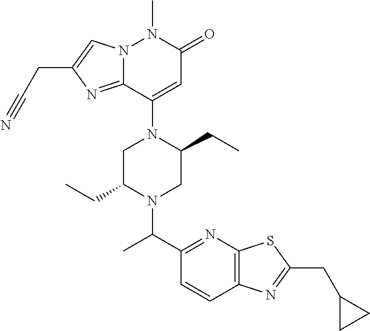 [SMILES: CC[C@H]1CN(C(C)c2ccc3nc(CC4CC4)sc3n2)[C@H](CC)CN1c1cc(=O)n(C)n2cc(CC#N)nc12]

### Step A: 2-cyclopropyl-N-(2,6-dichloropyridin-3-yl)acetamide

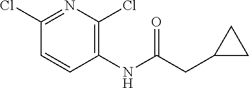 [SMILES: O=C(CC1CC1)Nc1ccc(Cl)nc1Cl]

To a solution of 2,6-dichloropyridin-3-amine (1 g, 6.1 mmol) in DMF (15 mL) were added HATU (3.5 g, 9.2 mmol), DIPEA (2.4 g, 18.5 mol) and 2-cyclopropylacetic acid (1.2 g, 12.2 mol). The mixture was stirred at 50° C. for overnight. The reaction was diluted with EA and washed with water. The organic layer was separated, dried by Na₂SO₄, filtered, and concentrated to dryness. The resulting residue was purified by flash column chromatography to give the titled compound (1 g, 66%). MS: M/e 245 (M+1)⁺.

### Step B: 5-chloro-2-(cyclopropylmethyl)thiazolo[54-b]pyridine

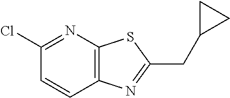 [SMILES: Clc1ccc2nc(CC3CC3)sc2n1]

To a solution of 2-cyclopropyl-N-(2,6-dichloropyridin-3-yl)acetamide (1 g, 4.1 mmol) in toluene (15 mL) was added lawesson reagent (0.85 g, 2.1 mmol) and the resulting mixture was stirred at 100° C. for 1 hours. The reaction mixture was concentrated and purified by flash column chromatography to give the titled compound (0.3 g). MS: M/e 225 (M+1)⁺.

### Step C: 1-(2-(cyclopropylmethyl)thiazolo[5,4-b]pyridin-5-yl)ethan-1-one

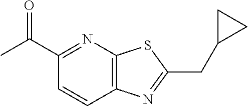 [SMILES: CC(=O)c1ccc2nc(CC3CC3)sc2n1]

A mixture of 5-chloro-2-(cyclopropylmethyl)thiazolo[5,4-b]pyridine (0.3 g, 1.3 mmol), tributyl(1-ethoxyvinyl)stannane (0.72 g, 2 mmol) and Pd(PPh₃)₂Cl₂ (91 mg, 0.57 mmol) in toluene (10 mL) was stirred at 100° C. under N₂ for 16 hours. The solution was diluted with ethyl acetate and washed with water. The combined organic layers were washed with brine, dried over Na₂SO₄ and the resulting residue was purified by flash column chromatography to give the titled compound (0.16 g, 51%). MS: M/e 232(M+1)⁺

### Step D: 1-(2-(cyclopropylmethyl)thiazolo[5,4-b]pyridin-5-yl)ethan-1-ol

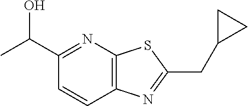 [SMILES: CC(O)c1ccc2nc(CC3CC3)sc2n1]

To a solution of 1-(2-(cyclopropylmethyl)thiazolo[5,4-b]pyridin-5-yl)ethan-1-one (0.16 g, 0.68 mmol) in MeOH (5 mL) was added NaBH₄ (26 mg, 0.68 mmol) at room temperature and the resulting mixture was stirred at room temperature for 5 min. The reaction mixture was diluted with DCM and washed with water, dried over Na₂SO₄, concentrated to give the titled compound (140 mg, 87%). MS: M/e 234 (M+1)⁺.

### Step E: 2-(8-((2S,5R)-4-(1-(2-(cyclopropylmethyl)thiazolo[5,4-b]pyridin-5-yl)ethyl)-2,5-diethylpiperazin-1-yl)-5-methyl-6-oxo-5,6-dihydroimidazo[1,2-b]pyridazin-2-yl)acetonitrile

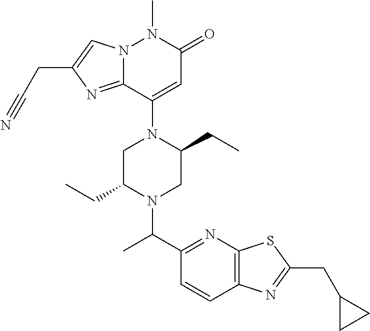 [SMILES: CC[C@H]1CN(C(C)c2ccc3nc(CC4CC4)sc3n2)[C@H](CC)CN1c1cc(=O)n(C)n2cc(CC#N)nc12]

To a solution of 2-(8-((2S,5R)-2,5-diethylpiperazin-1-yl)-5-methyl-6-oxo-5,6-dihydroimidazo[1,2-b]pyridazin-2-yl)acetonitrile (50 mg, 0.15 mmol) in CH₃CN (3 mL) and was added 1-(2-(cyclopropylmethyl)thiazolo[5,4-b]pyridin-5-yl)ethan-1-ol (71 mg, 0.3 mmol), (cyanomethyl)trimethylphosphonium iodide (111 mg, 0.45 mmol) and DIPEA (196 mg, 1.5 mmol). The resulting mixture was stirred at 105° C. overnight. The reaction solvent was removed under reduce pressure. The resulting residue was purified by flash column chromatography to give the titled compound (crude), which was further purified to give the titled Compound A110a (11 mg) and Compound A110b (11 mg) by Prep-HPLC (Method A).

Compound A110a (the earlier peak): ¹H NMR (400 MHz, CD₃OD) δ 8.22 (d, J=8.2 Hz, 1H), 7.80-7.73 (m, 2H), 5.58 (s, 1H), 3.91 (s, 2H), 3.87 (d, J=6.3 Hz, 1H), 3.71 (s, 3H), 3.52 (d, J=13.0 Hz, 1H), 3.37-3.30 (m, 2H), 3.16 (d, J=10.8 Hz, 1H), 3.02 (d, J=7.0 Hz, 2H), 2.78 (d, J=12.2 Hz, 1H), 2.26 (d, J=12.2 Hz, 1H), 2.07-1.47 (m, 4H), 1.38 (d, J=6.4 Hz, 3H), 1.30-1.15 (m, 1H), 1.02 (t, J=7.0 Hz, 3H), 0.75-0.53 (m, 5H), 0.39 (d, J=4.6 Hz, 2H) ppm. MS: M/e 545 (M+1)⁺.

Compound A110b (the later peak): ¹H NMR (400 MHz, CD₃OD) δ 8.23 (d, J=8.4 Hz, 1H), 7.76 (s, 1H), 7.69 (d, J=8.5 Hz, 1H), 5.59 (s, 1H), 4.13-4.01 (m, 1H), 3.90 (s, 2H), 3.71 (s, 3H), 3.36-3.30 (m, 3H), 3.08-2.89 (m, 4H), 2.48-2.31 (m, 1H), 2.14-1.53 (m, 4H), 1.42 (d, J=6.5 Hz, 3H), 1.35-1.06 (m, 1H), 0.93 (t, J=7.3 Hz, 3H), 0.78-0.60 (m, 5H), 0.39 (d, J=4.7 Hz, 2H) ppm. MS: M/e 545 (M+1)⁺.

Compound A111: 2-(cyanomethyl)-8-((2S,5R)-2,5-diethyl-4-(1-(2-methylbenzo[d]thiazol-6-yl)ethyl)piperazin-1-yl)-5-methyl-6-oxo-5,6-dihydroimidazo[1,2-b]pyridazine-7-carbonitrile

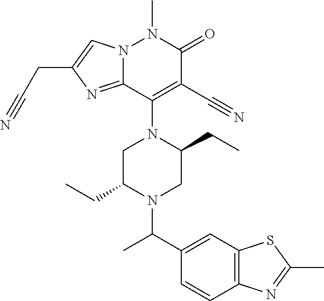 [SMILES: CC[C@H]1CN(C(C)c2ccc3nc(C)sc3c2)[C@H](CC)CN1c1c(C#N)c(=O)n(C)n2cc(CC#N)nc12]

### Step A: tert-butyl (2R,5S)-4-(7-bromo-2-(cyanomethyl)-5-methyl-6-oxo-5,6-dihydroimidazo[1,2-b]pyridazin-8-yl)-2,5-diethylpiperazine-1-carboxylate

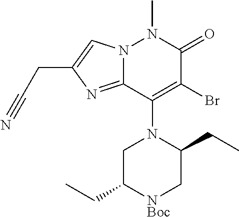 [SMILES: CC[C@@H]1CN(c2c(Br)c(=O)n(C)n3cc(CC#N)nc23)[C@@H](CC)CN1C(=O)OC(C)(C)C]

To a solution of tert-butyl (2R,5S)-4-(2-(cyanomethyl)-5-methyl-6-oxo-5,6-dihydroimidazo[1,2-b]pyridazin-8-yl)-2,5-diethylpiperazine-1-carboxylate (500 mg, 1.16 mmol) in CH₃CN (3 mL) and was added NBS (248 mg, 1.4 mmol). The resulting mixture was stirred for 5 mins at room temperature. The reaction solvent was removed under reduce pressure. The resulting residue was purified by flash column chromatography to give the titled compound (510 mg, 84%). MS: M/e 507 (M+1)⁺.

### Step B: tert-butyl (2R,5S)-4-(7-cyano-2-(cyanomethyl)-5-methyl-6-oxo-5,6-dihydroimidazo[1,2-b]pyridazin-8-yl)-2,5-diethylpiperazine-1-carboxylate

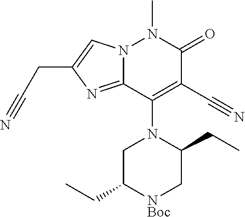 [SMILES: CC[C@@H]1CN(c2c(C#N)c(=O)n(C)n3cc(CC#N)nc23)[C@@H](CC)CN1C(=O)OC(C)(C)C]

To a solution of tert-butyl (2R,5S)-4-(7-bromo-2-(cyanomethyl)-5-methyl-6-oxo-5,6-dihydroimidazo[1,2-b]pyridazin-8-yl)-2,5-diethylpiperazine-1-carboxylate (300 mg, 0.59 mmol), Zn(CN)₂ (350 mg, 2.9 mmol) and Pd(PPh₃)₄(124 mg, 0.18 mmol) in DMF (3 mL). The resulting mixture was stirred at 100° C. overnight. The reaction mixture was diluted with DCM and washed with water, dried over Na₂SO₄ and concentrated. The resulting residue was purified by flash column chromatography to give the titled compound (280 mg). MS: M/e 454 (M+1)⁺

### Step C: 2-(cyanomethyl)-8-((2S,5R)-2,5-diethylpiperazin-1-yl)-5-methyl-6-oxo-5,6-dihydroimidazo[1,2-b]pyridazine-7-carbonitrile

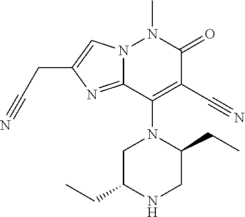 [SMILES: CC[C@@H]1CN(c2c(C#N)c(=O)n(C)n3cc(CC#N)nc23)[C@@H](CC)CN1]

To a solution of tert-butyl (2R,5S)-4-(7-cyano-2-(cyanomethyl)-5-methyl-6-oxo-5,6-dihydroimidazo[1,2-b]pyridazin-8-yl)-2,5-diethylpiperazine-1-carboxylate (0.28 g, 1.8 mmol) in DCM (10 mL) was added TFA (3 ml). The reaction mixture was stirred at RT for 3 hours, concentrated to dryness. The resulting residue was dissolved into water. The aqueous layer was adjusted pH to 9-10 with saturated Na₂CO₃ aq. and extracted with DCM/IPA (1/4). The resulting residue was purified by flash column chromatography to give the titled compound to give the titled compound (120 mg, 61%). MS: M/e 354 (M+1)⁺.

### Step D: 2-(cyanomethyl)-8-((2S,5R)-2,5-diethyl-4-(1-(2-methylbenzo[d]thiazol-6-yl)ethyl)piperazin-1-yl)-5-methyl-6-oxo-5,6-dihydroimidazo[1,2-b]pyridazine-7-carbonitrile

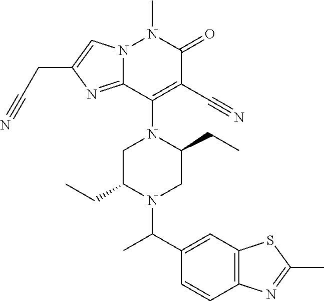 [SMILES: CC[C@H]1CN(C(C)c2ccc3nc(C)sc3c2)[C@H](CC)CN1c1c(C#N)c(=O)n(C)n2cc(CC#N)nc12]

To a solution of 2-(cyanomethyl)-8-((2S,5R)-2,5-diethylpiperazin-1-yl)-5-methyl-6-oxo-5,6-dihydroimidazo[1,2-b]pyridazine-7-carbonitrile (40 mg, 0.11 mmol) in CH₃CN (3 mL) and was added 1-(2-methylbenzo[d]thiazol-6-yl)ethan-1-ol (65 mg, 0.33 mmol), (cyanomethyl)trimethylphosphonium iodide (80 mg, 0.45 mmol) and DIPEA (150 mg, 1.1 mmol). The resulting mixture was stirred at 105° C. overnight. The reaction solvent was removed under reduce pressure. The resulting residue was purified by flash column chromatography to give the titled compound (crude), which was further purified to give the titled compound (10 mg) by Prep-HPLC (Method A). ¹H NMR (400 MHz, CD₃OD) δ 7.99-7.91 (m, 1H), 7.89-7.76 (m, 2H), 7.59-7.52 (m, 1H), 5.97-5.05 (m, 1H), 4.78-4.26 (m, 1H), 4.15-3.86 (m, 3H), 3.84-3.68 (m, 4H), 3.27-2.90 (m, 2H), 2.82 (s, 3H), 2.55-2.25 (m, 1H), 2.24-1.51 (m, 4H), 1.43-1.31 (m, 3H), 0.98-0.84 (m, 3H), 0.75-0.47 (m, 3H) ppm. MS: M/e 529 (M+1)⁺.

Compound A112: 2-(cyanomethyl)-8-((2S,5R)-2,5-diethyl-4-(1-(2-methylthiazolo[5,4-b]pyridin-5-yl)ethyl)piperazin-1-yl)-5-methyl-6-oxo-5,6-dihydroimidazo[1,2-b]pyridazine-7-carbonitrile

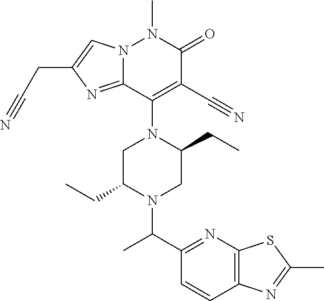 [SMILES: CC[C@H]1CN(C(C)c2ccc3nc(C)sc3n2)[C@H](CC)CN1c1c(C#N)c(=O)n(C)n2cc(CC#N)nc12]

A mixture of 2-(7-bromo-8-((2S,5R)-2,5-diethyl-4-(1-(2-methylthiazolo[5,4-b]pyridin-5-yl)ethyl)piperazin-1-yl)-5-methyl-6-oxo-5,6-dihydroimidazo[1,2-b]pyridazin-2-yl)acetonitrile (45 mg, 0.077 mmol), Zn(CN)₂(85 mg, 0.726 mmol) and Pd(PPh₃)₄(45 mg, 0.039 mmol) in DMF (1 mL) was stirred at 100° C. under N₂ for 16 hours. The mixture was diluted with EA (10 mL), washed with brine (5 mL×3), dried over Na₂SO₄, filtered and concentrated to dryness. The resulting residue was purified by column chromatography and further separated into Compound A112a (11 mg) and Compound A112b (10 mg) by Prep-HPLC(Method A).

Compound A112a (the earlier peak): ¹H NMR (400 MHz, CD₃OD) δ 8.23 (d, J=8.4 Hz, 1H), 7.89 (s, 1H), 7.78 (d, J=8.4 Hz, 1H), 6.05-5.41 (m, 1H), 4.50-3.99 (m, 2H), 3.96 (s, 2H), 3.93-3.83 (m, 1H), 3.75 (s, 3H), 3.27-2.99 (m, 2H), 2.86 (s, 3H), 2.38-2.22 (m, 1H), 2.17-1.47 (m, 4H), 1.40 (d, J=6.8 Hz, 3H), 1.02-0.80 (m, 3H), 0.78-0.62 (m, 3H) ppm. MS: M/e 530 (M+1)⁺.

Compound A112b (the later peak): ¹H NMR (400 MHz, CD₃OD) δ 8.25 (d, J=8.4 Hz, 1H), 7.89 (s, 1H), 7.75 (d, J=8.4 Hz, 1H), 6.36-4.50 (m, 1H), 4.16-3.92 (m, 3H), 3.89-3.78 (m, 1H), 3.75 (s, 3H), 3.24-2.95 (m, 2H), 2.86 (s, 3H), 2.54-2.20 (m, 2H), 2.18-1.34 (m, 7H), 0.90 (t, J=7.2 Hz, 3H), 0.57 (t, J=7.2 Hz, 3H) ppm. MS: M/e 530 (M+1)⁺.

Compound A113: 2-(cyanomethyl)-8-((2S,5R)-5-ethyl-2-methyl-4-(1-(2-methylthiazolo[5,4-b]pyridin-5-yl)ethyl)piperazin-1-yl)-5-methyl-6-oxo-5,6-dihydroimidazo[1,2-b]pyridazine-7-carbonitrile

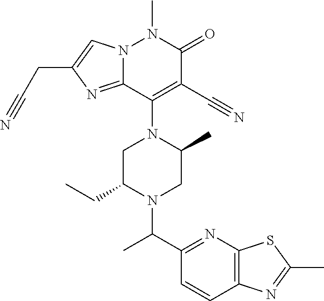 [SMILES: CC[C@@H]1CN(c2c(C#N)c(=O)n(C)n3cc(CC#N)nc23)[C@@H](C)CN1C(C)c1ccc2nc(C)sc2n1]

### Step A: 2-(7-bromo-8-((2S,5R)-5-ethyl-2-methyl-4-(1-(2-methylthiazolo[5,4-b]pyridin-5-yl)ethyl)piperazin-1-yl)-5-methyl-6-oxo-56-dihydroimidazo[1,2-b]pyridazin-2-yl)acetonitrile

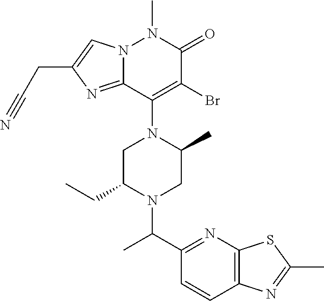 [SMILES: CC[C@@H]1CN(c2c(Br)c(=O)n(C)n3cc(CC#N)nc23)[C@@H](C)CN1C(C)c1ccc2nc(C)sc2n1]

To a solution of 2-(8-((2S,5R)-5-ethyl-2-methyl-4-(1-(2-methylthiazolo[5,4-b]pyridin-5-yl)ethyl)piperazin-1-yl)-5-methyl-6-oxo-5,6-dihydroimidazo[1,2-b]pyridazin-2-yl)acetonitrile (150 mg, 0.31 mmol) in MeCN (3 mL) was added NBS (60 mg, 0.34 mmol) at rt and the mixture was stirred at RT for 16 hours. The mixture was diluted with EA (10 mL), washed with brine (5 mL×3), dried over Na₂SO₄, filtered and concentrated under reduced pressure. The resulting residue was purified by flash column chromatography to give the titled compound (55 mg, 31%). MS: M/e 569, 571 (M+1)⁺.

### Step B: 2-(cyanomethyl)-8-((2S,5R)-5-ethyl-2-methyl-4-(1-(2-methylthiazolo[5,4-b]pyridin-5-yl)ethyl)piperazin-1-yl)-5-methyl-6-oxo-56-dihydroimidazo[1,2-b]pyridazine-7-carbonitrile

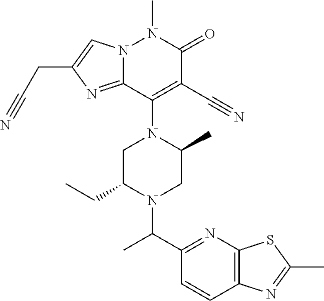 [SMILES: CC[C@@H]1CN(c2c(C#N)c(=O)n(C)n3cc(CC#N)nc23)[C@@H](C)CN1C(C)c1ccc2nc(C)sc2n1]

A mixture of 2-(7-bromo-8-((2S,5R)-5-ethyl-2-methyl-4-(1-(2-methylthiazolo[5,4-b]pyridin-5-yl)ethyl)piperazin-1-yl)-5-methyl-6-oxo-5,6-dihydroimidazo[1,2-b]pyridazin-2-yl)acetonitrile (45 mg, 0.079 mmol), Zn(CN)₂(37 mg, 0.317 mmol) and Pd(PPh₃)₄(18 mg, 0.016 mmol) in DMF (1 mL) was stirred at 100° C. under N₂ for 16 hours. The mixture was diluted with EA (10 mL), washed with brine (5 mL×3), dried over Na₂SO₄, filtered and concentrated to dryness. The resulting residue was purified and separated into Compound A113a (4 mg) and Compound A113b (4 mg) by Prep-HPLC (Method B).

Compound A113a (the earlier peak): ¹H NMR (400 MHz, CD₃OD) δ 8.25 (d, J=8.4 Hz, 1H), 7.87 (s, 1H), 7.75 (d, J=8.4 Hz, 1H), 6.22-5.03 (m, 2H), 4.87-4.20 (m, 1H), 4.07 (q, J=6.4 Hz, 1H), 3.99-3.91 (m, 1H), 3.90-3.78 (m, 1H), 3.74 (s, 3H), 3.17 (d, J=11.2 Hz, 1H), 2.99-2.90 (m, 1H), 2.86 (s, 3H), 2.48 (d, J=8.8 Hz, 1H), 1.64-1.48 (m, 5H), 1.44 (d, J=6.4 Hz, 3H), 0.64 (t, J=7.2 Hz, 3H) ppm. MS: M/e 516 (M+1)⁺.

Compound A113b (the later peak): ¹H NMR (400 MHz, CD₃OD) δ 8.23 (d, J=8.4 Hz, 1H), 7.88 (s, 1H), 7.80 (d, J=8.4 Hz, 1H), 5.47-5.01 (m, 2H), 4.15-4.00 m, 1H), 3.99-3.86 (m, 2H), 3.75 (s, 3H), 3.29-3.21 (m, 2H), 3.08-2.94 (m, 1H), 2.86 (s, 3H), 2.22 (d, J=12.8 Hz, 1H), 1.63-1.53 (m, 2H), 1.47-1.36 (m, 6H), 0.95 (t, J=7.2 Hz, 3H) ppm. MS: M/e 516 (M+1)⁺.

Compound A114: 2-(cyanomethyl)-8-((2S,5R)-2,5-diethyl-4-(1-(thiazolo[5,4-b]pyridin-5-yl)ethyl)piperazin-1-yl)-5-methyl-6-oxo-5,6-dihydroimidazo [1,2-b]pyridazine-7-carbonitrile

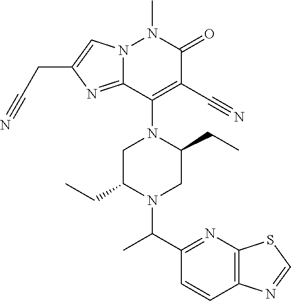 [SMILES: CC[C@H]1CN(C(C)c2ccc3ncsc3n2)[C@H](CC)CN1c1c(C#N)c(=O)n(C)n2cc(CC#N)nc12]

### Step A: 2-(7-bromo-8-((2S,5R)-2,5-diethyl-4-(1-(thiazolo[5,4-b]pyridin-5-yl)ethyl)piperazin-1-yl)-5-methyl-6-oxo-5,6-dihydroimidazo[1,2-b]pyridazin-2-yl)acetonitrile

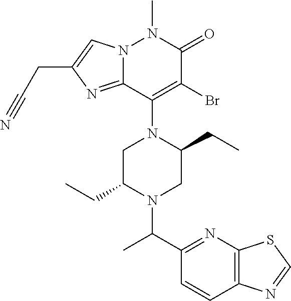 [SMILES: CC[C@H]1CN(C(C)c2ccc3ncsc3n2)[C@H](CC)CN1c1c(Br)c(=O)n(C)n2cc(CC#N)nc12]

To a solution of 2-(8-((2S,5R)-2,5-diethyl-4-(1-(thiazolo[5,4-b]pyridin-5-yl)ethyl)piperazin-1-yl)-5-methyl-6-oxo-5,6-dihydroimidazo[1,2-b]pyridazin-2-yl)acetonitrile (80 mg, 0.16 mmol) in CH₃CN (3 mL) and was added NBS (35 mg, 0.19 mmol). The resulting mixture was stirred for 5 mins at room temperature. The reaction solvent was removed under reduce pressure. The resulting residue was purified by flash column chromatography to give the titled compound (700 mg, 76%). MS: M/e 490 (M+1)⁺.

### Step B: 2-(cyanomethyl)-8-((2S,5R)-2,5-diethyl-4-(1-(thiazolo[5,4-b]pyridin-5-yl)ethyl)piperazin-1-yl)-5-methyl-6-oxo-5,6-dihydroimidazo[1,2-b]pyridazine-7-carbonitrile

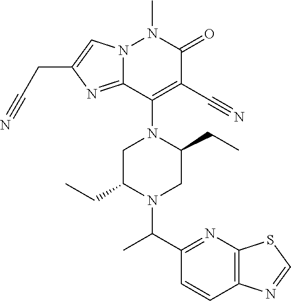 [SMILES: CC[C@H]1CN(C(C)c2ccc3ncsc3n2)[C@H](CC)CN1c1c(C#N)c(=O)n(C)n2cc(CC#N)nc12]

To a solution of 2-(7-bromo-8-((2S,5R)-2,5-diethyl-4-(1-(thiazolo[5,4-b]pyridin-5-yl)ethyl)piperazin-1-yl)-5-methyl-6-oxo-5,6-dihydroimidazo[1,2-b]pyridazin-2-yl)acetonitrile (70 mg, 0.12 mmol), Zn(CN)₂ (144 mg, 1.2 mmol) and Pd(PPh₃)₄(43 mg, 0.036 mmol) in DMF (3 mL). The resulting mixture was stirred at 100° C. overnight. The reaction mixture was diluted with DCM and washed with water, dried over Na₂SO₄ and concentrated. The resulting residue was purified by flash column chromatography to give the titled compound (crude), which was further purified to give the titled compound (7 mg) by Prep-HPLC(Method B). ¹H NMR (400 MHz, CD₃OD) δ 9.35 (s, 1H), 8.44 (t, J=8.0 Hz, 1H), 7.93-7.75 (m, 2H), 5.99-5.12 (m, 1H), 4.80-4.26 (m, 1H), 4.16-3.81 (m, 4H), 3.75 (s, 3H), 3.31-2.75 (m, 2H), 2.53-2.10 (m, 2H), 2.05-1.51 (m, 3H), 1.49-1.28 (m, 3H), 1.02-0.49 (m, 6H) ppm. MS: M/e 516 (M+1)⁺.

Compound A115: 2-(but-2-yn-1-yl)-8-((2S,5R)-2,5-diethyl-4-(1-(2-methylthiazolo[5,4-b]pyridin-5-yl)ethyl)piperazin-1-yl)-5-methylimidazo[1,2-b]pyridazin-6(5H)-one

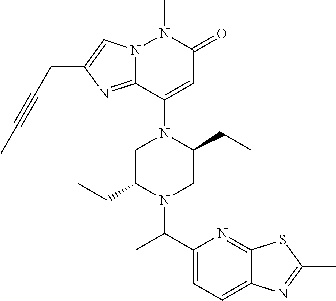 [SMILES: CC#CCc1cn2c(n1)c(N1C[C@@H](CC)N(C(C)c3ccc4nc(C)sc4n3)C[C@@H]1CC)cc(=O)n2C]

To a solution of 2-(but-2-yn-1-yl)-8-((2S,5R)-2,5-diethylpiperazin-1-yl)-5-methylimidazo[1,2-b]pyridazin-6(5H)-one (30 mg, 0.09 mmol) in CH₃CN (3 mL) and was added 1-(2-methylthiazolo[5,4-b]pyridin-5-yl)ethan-1-ol (17 mg, 0.09 mmol), (cyanomethyl)trimethylphosphonium iodide (64 mg, 0.26 mmol) and DIPEA (57 mg, 0.44 mmol). The resulting mixture was stirred at 105° C. overnight. The reaction solvent was removed under reduce pressure to afford crude product. The resulting residue was purified and separated into Compound A115a (9 mg) and Compound A115b (9 mg, 40%) by Prep-HPLC(Method A).

Compound A115a (the earlier peak): ¹H NMR (400 MHz, CD₃OD) δ 8.22 (d, J=8.5 Hz, 1H), 7.76 (d, J=8.5 Hz, 1H), 7.63 (s, 1H), 5.58 (s, 1H), 3.88 (q, J=6.8 Hz, 1H), 3.74 (s, 3H), 3.56-3.49 (m, 3H), 3.36-3.32 (m, 2H), 3.16 (d, J=11.2 Hz, 1H), 2.86 (s, 3H), 2.80 (d, J=8.8 Hz, 1H), 2.26 (d, J=12.0 Hz, 1H), 1.99-1.89 (m, 1H), 1.79 (t, J=2.6 Hz, 3H), 1.74-1.62 (m, 2H), 1.58-1.49 (m, 1H), 1.39 (d, J=6.7 Hz, 3H), 1.02 (t, J=7.3 Hz, 3H), 0.61 (t, J=7.3 Hz, 3H) ppm. MS: M/e 518(M+1)⁺.

Compound A115b (the later peak): ¹H NMR (400 MHz, CD₃OD) δ 8.23 (d, J=8.5 Hz, 1H), 7.71 (d, J=8.5 Hz, 1H), 7.63 (s, 1H), 5.58 (s, 1H), 4.06 (q, J=6.5 Hz, 1H), 3.74 (s, 3H), 3.52 (d, J=2.3 Hz, 2H), 3.41-3.32 (m, 3H), 2.99 (d, J=3.2 Hz, 2H), 2.86 (s, 3H), 2.39 (d, J=8.7 Hz, 1H), 2.11-2.00 (m, 1H), 1.89-1.81 (m, 1H), 1.78 (t, J=2.5 Hz, 3H), 1.63-1.51 (m, 2H), 1.43 (d, J=6.6 Hz, 3H), 0.93 (t, J=7.4 Hz, 3H), 0.69 (t, J=7.4 Hz, 3H) ppm. MS: M/e 518(M+1)⁺.

Compound A116: 2-(but-2-yn-1-yl)-8-((2S,5R)-2,5-diethyl-4-(1-(7-fluoro-2-methylbenzo[d]thiazol-6-yl)ethyl)piperazin-1-yl)-5-methylimidazo[1,2-b]pyridazin-6(5H)-one

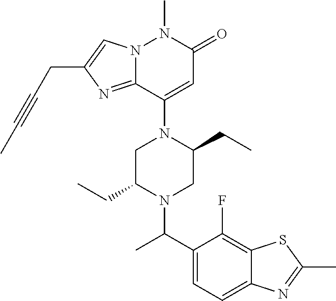 [SMILES: CC#CCc1cn2c(n1)c(N1C[C@@H](CC)N(C(C)c3ccc4nc(C)sc4c3F)C[C@@H]1CC)cc(=O)n2C]

To a solution of 2-(but-2-yn-1-yl)-8-((2S,5R)-2,5-diethylpiperazin-1-yl)-5-methylimidazo[1,2-b]pyridazin-6(5H)-one (100 mg, 0.20 mmol) (100 mg, 0.29 mmol) in CH₃CN (5 mL) was added 1-(5-fluorobenzo[d]thiazol-6-yl)ethan-1-ol (122 mg, 0.58 mmol), (cyanomethyl)trimethylphosphonium iodide (213 mg, 0.88 mmol) and DIPEA (378 mg, 2.9 mmol). The resulting mixture was stirred at 105° C. overnight. The reaction solvent was removed under reduce pressure. The resulting residue was purified by flash column chromatography to give the titled compound (crude), which was further separated into Compound A116a (19 mg) and Compound A116b (11 mg) by Prep-HPLC(Method B).

Compound A116a (the earlier peak):¹H NMR (400 MHz, CD₃OD) δ 7.75 (d, J=8.5 Hz, 1H), 7.70-7.64 (m, 1H), 7.62 (s, 1H), 5.57 (s, 1H), 4.84-4.45 (m, 1H), 4.30 (q, J=6.4 Hz, 1H), 3.73 (s, 3H), 3.51 (d, J=2.2 Hz, 2H), 3.25 (d, J=3.3 Hz, 2H), 3.03-2.91 (m, 2H), 2.85 (s, 3H), 2.38 (s, 1H), 2.14-1.80 (m, 2H), 1.78 (t, J=2.5 Hz, 3H), 1.62-1.48 (m, 2H), 1.42 (d, J=6.5 Hz, 3H), 0.94 (t, J=7.3 Hz, 3H), 0.69 (t, J=7.4 Hz, 3H) ppm. MS: M/e 535 (M+1)⁺.

Compound A116b (the later peak):¹H NMR (400 MHz, CD₃OD) δ 7.81-7.67 (m, 2H), 7.62 (s, 1H), 5.57 (s, 1H), 4.82-4.30 (m, 1H), 4.16 (q, J=6.4 Hz, 1H), 3.73 (s, 3H), 3.51 (t, J=11.2 Hz, 3H), 3.27-3.08 (m, 2H), 2.84 (s, 3H), 2.74 (d, J=9.7 Hz, 1H), 2.32 (d, J=12.3 Hz, 1H), 1.95 (s, 1H), 1.78 (t, J=2.5 Hz, 3H), 1.74-1.51 (m, 3H), 1.38 (d, J=6.6 Hz, 3H), 1.02 (t, J=7.3 Hz, 3H), 0.58 (t, J=7.2 Hz, 3H) ppm. MS: M/e 535(M+1)⁺.

Compound A117: 2-(but-2-yn-1-yl)-8-((2S,5R)-2,5-diethyl-4-(1-(7-fluorobenzo[d]thiazol-6-yl)ethyl)piperazin-1-yl)-5-methylimidazo[1,2-b]pyridazin-6(5H)-one

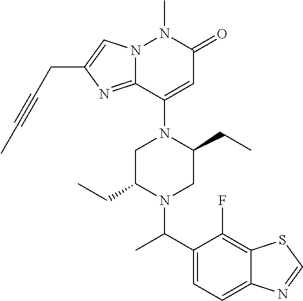 [SMILES: CC#CCc1cn2c(n1)c(N1C[C@@H](CC)N(C(C)c3ccc4ncsc4c3F)C[C@@H]1CC)cc(=O)n2C]

A solution of 2-(but-2-yn-1-yl)-8-((2S,5R)-2,5-diethylpiperazin-1-yl)-5-methylimidazo[1,2-b]pyridazin-6(5H)-one (100 mg, 0.29 mmol), 1-(7-fluorobenzo[d]thiazol-6-yl)ethan-1-ol (116 mg, 0.59 mmol), (cyanomethyl)trimethylphosphonium iodide (214 mg, 0.88 mmol) and DIPEA (378 mg, 2.93 mmol) in CH₃CN (3 ml) was stirred at 100° C. overnight. The reaction was diluted with EtOAc (10 ml) and washed with brine (10 ml). The organic layer was concentrated under reduced pressure. The resulting residue was purified by flash column chromatography to give the titled Compound A117, which was further separated into Compound A117a (14 mg) and Compound A117b (16 mg) by Prep-HPLC(Method A).

Compound A117a (the earlier peak): ¹H NMR (400 MHz, DMSO-d₆) δ 9.41 (s, 1H), 7.95 (d, J=8.4 Hz, 1H), 7.68 (d, J=7.4 Hz, 1H), 7.64 (s, 1H), 5.42 (s, 1H), 4.21 (q, J=6.5 Hz, 1H), 3.56 (s, 3H), 3.45 (s, 2H), 3.28 (s, 1H), 3.08 (d, J=10.7 Hz, 1H), 2.91-2.75 (m, 2H), 2.47 (s, 1H), 2.27-2.20 (m, 1H), 1.99-1.85 (m, 1H), 1.71 (s, 3H), 1.67-1.57 (m, 1H), 1.47-1.38 (m, 2H), 1.34 (d, J=6.4 Hz, 3H), 0.82 (t, J=6.9 Hz, 3H), 0.59 (t, J=6.9 Hz, 3H) ppm. MS: M/e 521 (M+1)⁺.

Compound A117b (the later peak): ¹H NMR (400 MHz, DMSO-d₆) δ 9.40 (s, 1H), 7.92 (d, J=8.5 Hz, 1H), 7.72 (t, J=7.6 Hz, 1H), 7.65 (s, 1H), 5.41 (s, 1H), 4.06 (q, J=6.5 Hz, 1H), 3.56 (s, 3H), 3.49-3.43 (m, 2H), 3.35-3.28 (m, 2H), 3.04 (d, J=9.8 Hz, 1H), 2.65-2.48 (m, 1H), 2.47 (s, 1H), 2.17 (d, J=11.9 Hz, 1H), 1.89-1.75 (m, 1H), 1.72 (t, J=2.6 Hz, 3H), 1.60-1.37 (m, 3H), 1.29 (d, J=6.4 Hz, 3H), 0.93 (t, J=6.9 Hz, 3H), 0.46 (t, J=6.9 Hz, 3H) ppm. MS: M/e 521 (M+1)⁺.

Compound A118: 2-(8-((2S,5R)-2-ethyl-5-methyl-4-(1-(2-methylbenzo[d]thiazol-6-yl)ethyl)piperazin-1-yl)-5-methyl-6-oxo-5,6-dihydroimidazo[1,2-b]pyridazin-2-yl)acetonitrile

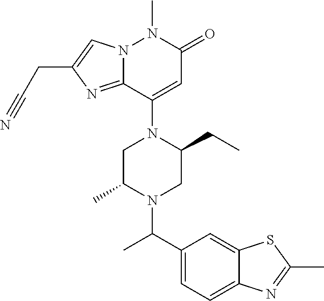 [SMILES: CC[C@H]1CN(C(C)c2ccc3nc(C)sc3c2)[C@H](C)CN1c1cc(=O)n(C)n2cc(CC#N)nc12]

A mixture of 2-(8-((2S,5R)-2-ethyl-5-methylpiperazin-1-yl)-5-methyl-6-oxo-5,6-dihydroimidazo[1,2-b]pyridazin-2-yl)acetonitrile (31.4 mg, 0.1 mmol), 1-(2-methylbenzo[d]thiazol-6-yl)ethan-1-ol (38.6 mg, 0.2 mmol), (cyanomethyl)trimethylphosphonium iodide (72.9 mg, 0.3 mmol) and DIPEA (64.5 mg, 0.5 mmol) in CH₃CN (4 mL) was stirred at 100° C. overnight in a sealed tube. The reaction mixture was diluted with EtOAc (10 mL), washed with brine, dried over Na₂SO₄, concentrated and purified by Prep-HPLC (Method A) to give the titled compound (6 mg). ¹H NMR (400 MHz, CD₃OD) δ 7.96-7.92 (m, 1H), 7.87-7.81 (m, 1H), 7.79-7.76 (m, 1H), 7.59-7.52 (m, 1H), 5.62-5.56 (m, 1H), 3.98-3.91 (m, 2H), 3.83-3.76 (m, 0.5H), 3.75-3.71 (m, 3H), 3.68-3.58 (m, 1.5H), 3.49-3.38 (m, 0.5H), 3.35-3.31 (m, 2H), 3.07-2.86 (m, 1.5H), 2.85-2.80 (m, 3H), 2.74-2.67 (m, 0.5H), 2.38-2.30 (m, 0.5H), 2.19-1.60 (m, 2H), 1.43-1.34 (m, 3H), 1.18-1.11 (m, 1.5H), 1.01-0.91 (m, 3H), 0.63-0.53 (m, 1.5H) ppm. MS: M/e 490 (M+1)⁺.

Compound A119: 2-(8-((2S,5R)-2-ethyl-4-(1-(7-fluoro-2-methylbenzo[d]thiazol-6-yl)ethyl)-5-methylpiperazin-1-yl)-5-methyl-6-oxo-5,6-dihydroimidazo[1,2-b]pyridazin-2-yl)acetonitrile

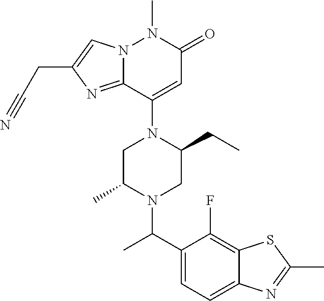 [SMILES: CC[C@H]1CN(C(C)c2ccc3nc(C)sc3c2F)[C@H](C)CN1c1cc(=O)n(C)n2cc(CC#N)nc12]

A mixture of 2-(8-((2S,5R)-2-ethyl-5-methylpiperazin-1-yl)-5-methyl-6-oxo-5,6-dihydroimidazo[1,2-b]pyridazin-2-yl)acetonitrile (31.4 mg, 0.1 mmol), 1-(7-fluoro-2-methylbenzo[d]thiazol-6-yl)ethan-1-ol (41.4 mg, 0.2 mmol), (cyanomethyl)trimethylphosphonium iodide (72.9 mg, 0.3 mmol) and DIPEA (64.5 mg, 0.5 mmol) in CH₃CN (4 mL) was stirred at 100° C. overnight in a sealed tube. The reaction mixture was diluted with EtOAc (10 mL), washed with brine, dried over Na₂SO₄, concentrated to dryness. The resulting residue was purified and separated into Compound A119a (5 mg) and Compound A119b (5 mg) by Prep-HPLC (Method A).

Compound A119a (the earlier peak): ¹H NMR (400 MHz, CD₃OD) δ 7.83-7.69 (m, 3H), 5.60 (s, 1H), 5.3-4.4 (m, 2H), 4.07 (q, J=6.4 Hz, 1H), 3.93 (s, 2H), 3.73 (s, 3H), 3.69-3.57 (m, 2H), 2.84 (s, 3H), 2.80-2.71 (m, 1H), 2.37-2.28 (m, 1H), 2.02-1.88 (m, 1H), 1.79-1.62 (m, 1H), 1.41 (d, J=6.5 Hz, 3H), 1.15 (d, J=6.5 Hz, 3H), 0.61 (t, J=7.4 Hz, 3H) ppm. MS: M/e 508 (M+1)⁺.

Compound A119b (the later peak): ¹H NMR (400 MHz, CD₃OD) δ 7.85-7.64 (m, 3H), 5.59 (s, 1H), 5.4-4.4 (m, 2H), 4.21 (q, J=6.4 Hz, 1H), 3.93 (s, 2H), 3.73 (s, 3H), 3.45-3.37 (m, 1H), 3.06-2.94 (m, 2H), 2.92-2.86 (m, 1H), 2.85 (s, 3H), 2.16-2.01 (m, 1H), 1.93-1.82 (m, 1H), 1.40 (d, J=6.4 Hz, 3H), 1.00 (d, J=6.4 Hz, 3H), 0.94 (t, J=7.6 Hz, 3H) ppm. MS: M/e 508 (M+1)⁺.

Compound A120: 2-(8-((2S,5R)-2-ethyl-5-methyl-4-(1-(2-methylbenzo[d]thiazol-6-yl)propyl)piperazin-1-yl)-5-methyl-6-oxo-5,6-dihydroimidazo[, 2-b]pyridazin-2-yl)acetonitrile

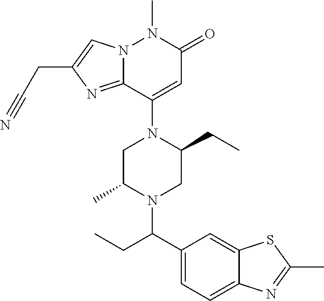 [SMILES: CCC(c1ccc2nc(C)sc2c1)N1C[C@H](CC)N(c2cc(=O)n(C)n3cc(CC#N)nc23)C[C@H]1C]

### Step A: 2-methylbenzo[d]thiazole-6-carbonitrile

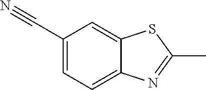

To a stirred solution of 6-bromo-2-methylbenzo[d]thiazole (2.28 g, 10 mmol) in DMF (40 mL) was added ZnCN₂ (3.51 g, 30 mmol), followed by Pd(PPh₃)₄(1.15 g, 1 mmol). After the addition, the reaction mixture was stirred overnight at 100° C. under N₂. The reaction mixture was poured into H₂O (50 mL) and extracted with EtOAc (40 mL×3). The combined organic layers were washed with brine, dried over Na₂SO₄, concentrated and purified by flash column chromatography to give the titled compound (1.3 g, 75%). MS: M/e 175 (M+1)⁺.

### Step B: 2-methylbenzo[d]thiazole-6-carbaldehyde

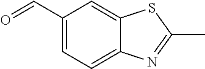

To a stirred solution of 2-methylbenzo[d]thiazole-6-carbonitrile (348 mg, 2 mmol) in THF (8 mL) was added dropwise DIBAL-H (1.0 M, 4 mL, 4 mmol) at 0° C. After stirred for 30 min, the reaction was quenched with aq.NH₄Cl, extracted with EtOAc (30 mL×3). The combined organic layers were washed with brine, dried over Na₂SO₄, concentrated and purified by flash column chromatography to give the titled compound (215 mg, 61%). MS: M/e 178 (M+1)⁺.

### Step C: 1-(2-methylbenzo[d]thiazol-6-yl)propan-1-ol

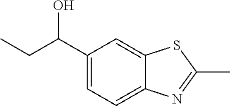

To a stirred solution of 2-methylbenzo[d]thiazole-6-carbaldehyde (215 mg, 1.21 mmol) in THF (8 mL) was added dropwise EtMgBr (3.0 M, 0.5 mL, 1.45 mL) at 0° C. After the addition, the mixture was stirred for 20 min. The reaction was quenched with NH4Cl, extracted with EtOAc (10 mL×3). The combined organic layers were washed with brine, dried over Na₂SO₄, concentrated and purified by flash column chromatography to give the titled compound (182 mg, 73%). MS: M/e 208 (M+1)⁺.

### Step D: 2-(8-((2S,5R)-2-ethyl-5-methyl-4-(1-(2-methylbenzo[d]thiazol-6-yl)propyl)piperazin-1-yl)-5-methyl-6-oxo-5,6-dihydroimidazo[1,2-b]pyridazin-2-yl)acetonitrile

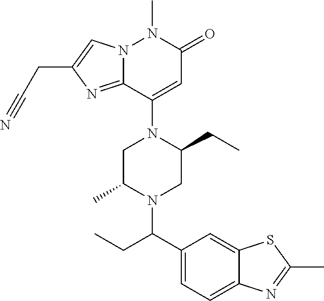 [SMILES: CCC(c1ccc2nc(C)sc2c1)N1C[C@H](CC)N(c2cc(=O)n(C)n3cc(CC#N)nc23)C[C@H]1C]

A mixture of 2-(8-((2S,5R)-2-ethyl-5-methylpiperazin-1-yl)-5-methyl-6-oxo-5,6-dihydroimidazo[1,2-b]pyridazin-2-yl)acetonitrile (31.4 mg, 0.1 mmol), 1-(2-methylbenzo[d]thiazol-6-yl)propan-1-ol (41.4 mg, 0.2 mmol), (cyanomethyl)trimethylphosphonium iodide (72.9 mg, 0.3 mmol) and DIPEA (64.5 mg, 0.5 mmol) in CH₃CN (4 mL) was stirred at 100° C. overnight in a sealed tube. The reaction mixture was diluted with EtOAc (10 mL), washed with brine, dried over Na₂SO₄, concentrated to dryness. The resulting residue was purified and separated into Compound A120a (4 mg) and Compound A120b (3.5 mg) by Prep-HPLC (Method A).

Compound A120a (the earlier peak): ¹H NMR (400 MHz, CD₃OD) δ 7.89 (s, 1H), 7.83 (d, J=8.4 Hz, 1H), 7.77 (s, 1H), 7.50 (d, J=8.4 Hz, 1H), 5.58 (s, 1H), 5.25-5.03 (m, 2H), 3.92 (s, 2H), 3.72 (s, 3H), 3.62-3.55 (m, 2H), 3.46-3.40 (m, 1H), 2.82 (s, 3H), 2.74-2.66 (m, 1H), 2.35-2.27 (m, 1H), 2.11-1.90 (m, 2H), 1.71-1.58 (m, 2H), 1.12 (d, J=6.4 Hz, 3H), 0.65 (t, J=7.2 Hz, 3H), 0.56 (t, J=7.2 Hz, 3H) ppm. MS: M/e 504 (M+1)⁺.

Compound A120b (the later peak): ¹H NMR (400 MHz, CD₃OD) δ 7.89 (s, 1H), 7.86 (d, J=8.4 Hz, 1H), 7.77 (s, 1H), 7.51 (dd, J=8.4, 1.2 Hz, 1H), 5.58 (s, 1H), 5.30-4.90 (m, 1H), 4.65-4.10 (m, 1H), 3.93 (s, 2H), 3.72 (s, 3H), 3.61-3.54 (m, 1H), 3.43-3.36 (m, 1H), 3.07-2.85 (m, 3H), 2.83 (s, 3H), 2.15-1.95 (m, 2H), 1.94-1.60 (m, 2H), 1.00-0.92 (m, 6H), 0.67 (t, J=7.2 Hz, 3H) ppm. MS: M/e 504 (M+1)⁺.

Compound A121: 2-(8-((2S,5R)-2-ethyl-5-methyl-4-(1-(2-methylthiazolo[5,4-b]pyridin-5-yl)ethyl)piperazin-1-yl)-5-methyl-6-oxo-5,6-dihydroimidazo [1,2-b]pyridazin-2-yl)acetonitrile

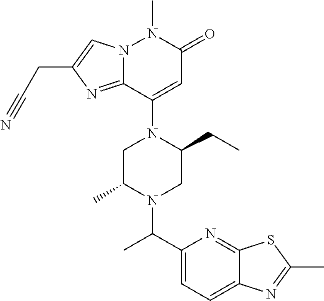 [SMILES: CC[C@H]1CN(C(C)c2ccc3nc(C)sc3n2)[C@H](C)CN1c1cc(=O)n(C)n2cc(CC#N)nc12]

A mixture of 2-(8-((2S,5R)-2-ethyl-5-methylpiperazin-1-yl)-5-methyl-6-oxo-5,6-dihydroimidazo[1,2-b]pyridazin-2-yl)acetonitrile (62.8 mg, 0.2 mmol), 1-(2-methylthiazolo[5,4-b]pyridin-5-yl)ethan-1-ol (77.6 mg, 0.4 mmol), (cyanomethyl)trimethylphosphonium iodide (146 mg, 0.6 mmol) and DIPEA (129 mg, 1 mmol) in CH₃CN (5 mL) was stirred at 100° C. overnight in a sealed tube. The reaction mixture was diluted with EtOAc (15 mL), washed with brine, dried over Na₂SO₄, concentrated to dryness. The resulting residue was purified by flash column chromatography to give the titled compound, which was further separated into Compound A121a (21 mg) and Compound A121b (22 mg) by Prep-HPLC (Method A).

Compound A121a (the earlier peak): ¹H NMR (400 MHz, CD₃OD) δ 8.22 (d, J=8.4 Hz, 1H), 7.78 (s, 1H), 7.73 (d, J=8.4 Hz, 1H), 5.61 (s, 1H), 5.30-4.21 (m, 2H), 3.93 (s, 2H), 3.84 (q, J=6.4 Hz, 1H), 3.73 (s, 3H), 3.69-3.60 (m, 2H), 2.90-2.81 (m, 4H), 2.31-2.24 (m, 1H), 2.02-1.89 (m, 1H), 1.79-1.66 (m, 1H), 1.43 (d, J=6.4 Hz, 3H), 1.17 (d, J=6.4 Hz, 3H), 0.64 (t, J=7.2 Hz, 3H) ppm. MS: M/e 491 (M+1)⁺.

Compound A121b (the later peak): ¹H NMR (400 MHz, CD₃OD) δ 8.20 (d, J=8.4 Hz, 1H), 7.84-7.67 (m, 2H), 5.57 (s, 1H), 5.35-4.20 (m, 2H), 4.00-3.87 (m, 3H), 3.70 (s, 3H), 3.49-3.39 (m, 1H), 3.05-2.95 (m, 2H), 2.92-2.78 (m, 4H), 2.15-2.01 (m, 1H), 1.92-1.78 (m, 1H), 1.38 (d, J=6.4 Hz, 3H), 1.01 (d, J=6.4 Hz, 3H), 0.90 (t, J=7.2 Hz, 3H) ppm. MS: M/e 491 (M+1)⁺.

Compound A122: 2-(8-((2S,5R)-5-ethyl-2-methyl-4-(1-(2-methylthiazolo[5,4-b]pyridin-5-yl)propyl)piperazin-1-yl)-5-methyl-6-oxo-5,6-dihydroimidazo[1,2-b]pyridazin-2-yl)acetonitrile

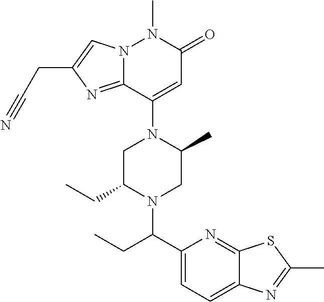 [SMILES: CCC(c1ccc2nc(C)sc2n1)N1C[C@H](C)N(c2cc(=O)n(C)n3cc(CC#N)nc23)C[C@H]1CC]

A mixture of 2-(8-((2S,5R)-5-ethyl-2-methylpiperazin-1-yl)-5-methyl-6-oxo-5,6-dihydroimidazo[1,2-b]pyridazin-2-yl)acetonitrile (31.4 mg, 0.1 mmol), 1-(2-methylthiazolo[5,4-b]pyridin-5-yl)propan-1-ol (41.6 mg, 0.2 mmol), (cyanomethyl)trimethylphosphonium iodide (72.9 mg, 0.3 mmol) and DIPEA (64.5 mg, 0.5 mmol) in CH₃CN (5 mL) was stirred at 100° C. overnight in a sealed tube. The reaction mixture was diluted with EtOAc (10 mL), washed with brine, dried over Na₂SO₄, concentrated to dryness. The resulting residue was purified by Pre-TLC (EtOAc) to give the titled compound, which was further separated into Compound A122a (4 mg) and Compound A122b (5 mg) by Prep-HPLC (Method B).

Compound A122a (the earlier peak): ¹H NMR (400 MHz, CD₃OD) δ 8.22 (d, J=8.4 Hz, 1H), 7.77 (s, 1H), 7.61 (d, J=8.4 Hz, 1H), 5.60 (s, 1H), 5.35-5.10 (m, 1H), 4.70-4.50 (m, 1H), 3.91 (s, 2H), 3.86 (dd, J=8.4, 5.4 Hz, 1H), 3.73 (s, 3H), 3.34-3.31 (m, 1H), 3.16-3.07 (m, 1H), 2.86 (s, 3H), 2.10-1.82 (m, 2H), 1.62-1.44 (m, 2H), 1.33 (d, J=6.4 Hz, 3H), 0.79-0.71 (m, 6H) ppm. MS: M/e 505 (M+1)⁺.

Compound A122b (the later peak): ¹H NMR (400 MHz, CD₃OD) δ 8.21 (d, J=8.4 Hz, 1H), 7.78 (s, 1H), 7.72 (d, J=8.4 Hz, 1H), 5.60 (s, 1H), 5.46-5.00 (m, 1H), 4.68-4.35 (m, 1H), 3.92 (s, 2H), 3.72 (s, 3H), 3.71-3.67 (m, 1H), 3.56-3.48 (m, 1H), 3.22-3.12 (m, 1H), 2.92-2.86 (m, 1H), 2.85 (s, 3H), 2.20-2.13 (m, 1H), 2.10-1.96 (m, 1H), 1.88-1.63 (m, 2H), 1.53-1.41 (m, 1H), 1.21 (d, J=6.4 Hz, 3H), 1.05 (t, J=7.2 Hz, 3H), 0.68 (t, J=7.2 Hz, 3H) ppm. MS: M/e 505 (M+1)⁺.

Compound A123: 8-((2S,5R)-2,5-diethyl-4-(1-(2-methylbenzo[d]thiazol-6-yl)ethyl)piperazin-1-yl)-5-methyl-6-oxo-5,6-dihydroimidazo[1,2-b]pyridazine-2-carbaldehyde O-methyl oxime

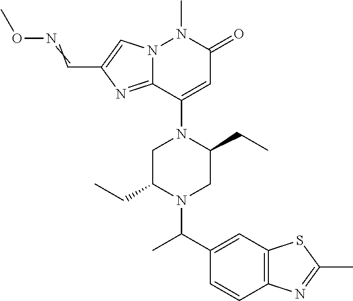

### Step A: tert-butyl (2R,5S)-2,5-diethyl-4-(2-formyl-5-methyl-6-oxo-5,6-dihydroimidazo[1,2-b]pyridazin-8-yl)piperazine-1-carboxylate

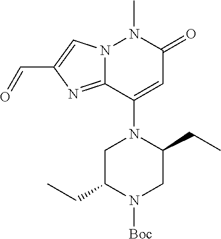 [SMILES: CC[C@@H]1CN(c2cc(=O)n(C)n3cc(C=O)nc23)[C@@H](CC)CN1C(=O)OC(C)(C)C]

To a solution of tert-butyl (2R,5S)-2,5-diethyl-4-(2-(hydroxymethyl)-5-methyl-6-oxo-5,6-dihydroimidazo[1,2-b]pyridazin-8-yl)piperazine-1-carboxylate (1.2 g, 2.86 mmol) in CH₂Cl₂ (20 mL) was added Dess-Martin reagent (1.5 g, 3.54 mmol) at rt and the mixture was stirred for 30 minutes. The mixture was diluted with CH₂Cl₂ (20 mL), washed with NaHCO₃ (aqueous solution, 20 mL×2), dried over Na₂SO₄, filtered and concentrated under reduced pressure. The resulting residue was purified by column chromatography to give the titled compound (1.1 g, 92%). MS: M/e 418 (M+1)⁺.

### Step B: tert-butyl (2R,5S)-2,5-diethyl-4-(2-((methoxyimino)methyl)-5-methyl-6-oxo-5,6-dihydroimidazo[1,2-b]pyridazin-8-yl)piperazine-1-carboxylate

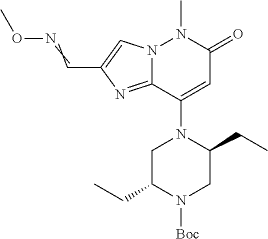 [SMILES: CC[C@@H]1CN(c2cc(=O)n(C)n3cc(C=NOC)nc23)[C@@H](CC)CN1C(=O)OC(C)(C)C]

A mixture of tert-butyl (2R,5S)-2,5-diethyl-4-(2-formyl-5-methyl-6-oxo-5,6-dihydroimidazo[1,2-b]pyridazin-8-yl)piperazine-1-carboxylate (600 mg, 1.44 mmol), O-methylhydroxylamine hydrochloride (360 mg, 4.31 mmol) and K₂CO₃ (600 mg, 4.31 mmol) in MeOH (10 mL) was stirred at rt for 3 hours. The mixture was diluted with EA (30 mL), washed with brine (10 mL×3), dried over Na₂SO₄, filtered and concentrated to give the titled compound (350 mg, 54%). MS: M/e 447 (M+1)⁺.

### Step C: 8-((2S,5R)-2,5-diethylpiperazin-1-yl)-5-methyl-6-oxo-5,6-dihydroimidazo[1,2-b]pyridazine-2-carbaldehyde O-methyl oxime

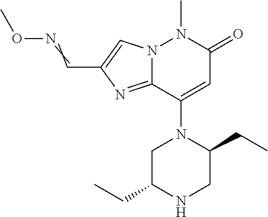 [SMILES: CC[C@@H]1CN(c2cc(=O)n(C)n3cc(C=NOC)nc23)[C@@H](CC)CN1]

To a solution of tert-butyl (2R,5S)-2,5-diethyl-4-(2-((methoxyimino)methyl)-5-methyl-6-oxo-5,6-dihydroimidazo[1,2-b]pyridazin-8-yl)piperazine-1-carboxylate (350 mg, 0.78 mmol) in MeOH (5 mL) was added HCl/dioxane (4 M, 2 mL) at rt and the mixture was stirred for 1 hour. The mixture was concentrated to dryness to give the titled compound (320 mg, crude). MS: M/e 347 (M+1)⁺.

### Step D: 8-((2S,5R)-2,5-diethyl-4-(1-(2-methylbenzo[d]thiazol-6-yl)ethyl)piperazin-1-yl)-5-methyl-6-oxo-5,6-dihydroimidazo[1,2-b]pyridazine-2-carbaldehyde O-methyl oxime

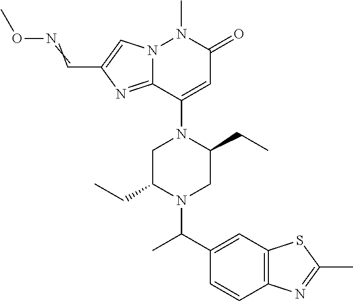

To a solution of 8-((2S,5R)-2,5-diethylpiperazin-1-yl)-5-methyl-6-oxo-5,6-dihydroimidazo[1,2-b]pyridazine-2-carbaldehyde O-methyl oxime (70 mg, 0.2 mmol), 1-(2-methylbenzo[d]thiazol-6-yl)ethan-1-ol (77 mg, 0.4 mmol) and (cyanomethyl)trimethylphosphonium iodide (150 mg, 0.6 mmol) in CH₃CN (1 mL) was added DIPEA (129 mg, 1.0 mmol). The mixture was stirred at 100° C. in a sealed tube for 16 hours. The mixture was cooled and diluted with EtOAc (10 mL), washed with brine (5 mL×3), dried over Na₂SO₄, filtered and concentrated under reduced pressure. The resulting residue was purified by flash column chromatography and further separated into Compound A123a (3 mg), Compound A123bc (10 mg) and Compound A123d (6 mg) by Prep-HPLC (Method A).

Compound A123a (the first peak): ¹H NMR (400 MHz, CD₃OD) δ 8.05 (s, 1H), 8.00 (s, 1H), 7.90 (s, 1H), 7.83 (d, J=8.4 Hz, 1H), 7.51 (d, J=7.2 Hz, 1H), 5.59 (s, 1H), 4.75-4.19 (m, 1H), 3.95-3.79 (m, 4H), 3.72 (s, 3H), 3.27-3.16 (m, 2H), 3.07-2.98 (m, 1H), 2.95-2.86 (m, 1H), 2.81 (s, 3H), 2.48-2.34 (m, 1H), 2.17-2.01 (m, 1H), 1.94-1.79 (m, 1H), 1.64-1.43 (m, 2H), 1.37 (d, J=6.4 Hz, 3H), 0.93 (t, J=7.2 Hz, 3H), 0.63 (t, J=7.2 Hz, 3H) ppm. MS: M/e 522 (M+1)⁺.

Compound A123bc (a mixture of second and third peak): ¹H NMR (400 MHz, CD₃OD) δ 8.33-7.34 (m, 5H), 5.67-5.51 (m, 1H), 4.81-4.14 (m, 1H), 4.10-3.80 (m, 4H), 3.77-3.71 (m, 3H), 3.57-3.42 (m, 1H), 3.23-2.85 (m, 2H), 2.84-2.76 (m, 3H), 2.76-2.59 (m, 1H), 2.49-2.28 (m, 1H), 2.16-1.49 (m, 4H), 1.41-1.31 (m, 3H), 1.06-0.87 (m, 3H), 0.73-0.50 (m, 3H) ppm. MS: M/e 522 (M+1)⁺.

Compound A123d (the fourth peak):¹H NMR (400 MHz, CD₃OD) δ 8.27 (s, 1H), 7.94 (s, 1H), 7.81 (d, J=8.4 Hz, 1H), 7.54 (d, J=8.4 Hz, 1H), 7.40 (s, 1H), 5.59 (s, 1H), 4.75-4.14 (m, 1H), 4.03 (s, 3H), 3.79-3.68 (m, 4H), 3.55-3.47 (m, 1H), 3.26-3.09 (m, 2H), 2.80 (s, 3H), 2.71-2.61 (m, 1H), 2.35 (d, J=12.4 Hz, 1H), 2.05-1.89 (m, 1H), 1.75-1.50 (m, 3H), 1.35 (d, J=6.4 Hz, 3H), 1.02 (t, J=7.2 Hz, 3H), 0.55 (t, J=7.2 Hz, 3H) ppm. MS: M/e 522 (M+1)⁺.

Compound A124: 8-((2S,5R)-2,5-diethyl-4-(1-(2-methylthiazolo[5,4-b]pyridin-5-yl)ethyl)piperazin-1-yl)-5-methyl-6-oxo-5,6-dihydroimidazo[1,2-b]pyridazine-2-carbaldehyde O-methyl oxime

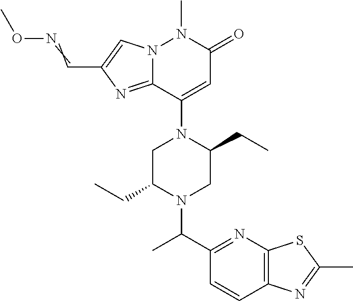

To a solution of 8-((2S,5R)-2,5-diethylpiperazin-1-yl)-5-methyl-6-oxo-5,6-dihydroimidazo[1,2-b]pyridazine-2-carbaldehyde O-methyl oxime (70 mg, 0.2 mmol), 1-(2-methylthiazolo[5,4-b]pyridin-5-yl)ethan-1-ol (77 mg, 0.4 mmol) and (cyanomethyl)trimethylphosphonium iodide (150 mg, 0.6 mmol) in CH₃CN (1 mL) was added DIPEA (129 mg, 1.0 mmol). The mixture was stirred at 100° C. in a sealed tube for 16 hours. The mixture was cooled and diluted with EtOAc (10 mL), washed with brine (5 mL×3), dried over Na₂SO₄, filtered and concentrated under reduced pressure. The resulting residue was purified by flash column chromatography and further separated into Compound A124a (3 mg), Compound A124bc (14 mg) and Compound A124d (7 mg) by Prep-HPLC (Method A).

Compound A124a (the first peak): ¹H NMR (400 MHz, CD₃OD) δ 8.20 (d, J=8.4 Hz, 1H), 8.06 (s, 1H), 8.02 (s, 1H), 7.74 (d, J=8.4 Hz, 1H), 5.59 (s, 1H), 4.72-4.12 (m, 1H), 3.99-3.80 (m, 4H), 3.73 (s, 3H), 3.58-3.48 (m, 1H), 3.27-3.12 (m, 2H), 2.91-2.73 (m, 4H), 2.26 (d, J=12.0 Hz, 1H), 2.00-1.83 (m, 1H), 1.81-1.47 (m, 3H), 1.38 (d, J=6.8 Hz, 3H), 1.00 (t, J=7.2 Hz, 3H), 0.60 (t, J=7.2 Hz, 3H) ppm. MS: M/e 523 (M+1)⁺.

Compound A124bc (a mixture of second and third peak): ¹H NMR (400 MHz, CD₃OD) δ 8.30-7.37 (m, 4H), 5.64-5.55 (m, 1H), 4.81-4.17 (m, 1H), 4.10-3.82 (m, 4H), 3.79-3.68 (m, 3H), 3.58-3.33 (m, 1H), 3.21-3.08 (m, 1H), 3.05-2.89 (m, 1H), 2.81-2.73 (m, 1H), 2.45-2.20 (m, 1H), 2.09-1.47 (m, 4H), 1.45-1.35 (m, 3H), 1.06-0.87 (m, 3H), 0.73-0.55 (m, 3H) ppm. MS: M/e 523 (M+1)⁺.

Compound A124d (the fourth peak): ¹H NMR (400 MHz, CD₃OD) δ 8.27 (s, 1H), 8.21 (d, J=8.4 Hz, 1H), 7.69 (d, J=8.4 Hz, 1H), 7.39 (s, 1H), 5.61 (s, 1H), 4.80-4.28 (m, 1H), 4.09-3.97 (m, 4H), 3.75 (s, 3H), 3.38-3.29 (m, 2H), 3.04-2.91 (m, 2H), 2.84 (s, 3H), 2.43-2.33 (m, 1H), 2.15-1.98 (m, 1H), 1.91-1.76 (m, 1H), 1.65-1.47 (m, 2H), 1.41 (d, J=6.4 Hz, 3H), 0.92 (t, J=7.2 Hz, 3H), 0.69 (t, J=7.2 Hz, 3H) ppm. MS: M/e 523 (M+1)⁺.

### Assay

### Biochemical DGK IC50 Assays

Enzymatic reactions of DGKζ, DGKα and DGKδ were performed using ADP-Glo assay with lipid micelle substrate. Full length DGKζ (in-house protein M1-V929 with SEQ ID No: 2) was expressed in baculovirus expression system. Full length DGKα (D21-10BG, SignalChem) and full length DGKδ (D23-10G, SignalChem) were purchased. Lipid micelle was prepared by dissolving DAG (Sigma, 317505-10MG) and PS (Sigma, P7769-100MG) with chloroform which was firtherly removed by rotary evaporation. The obtained residue was resuspended in buffer containing 25 mM HEPES pH 7.0, 0.5 mM EDTA and 160 mM Octyl β-D glucopyranoside by vigorous mixing and ultrasonic (IID, Scientz).

| | Final concentration in reaction | | |
|---|---|---|---|
| Enzyme | Enzyme (nM) | ATP (μM) | DAG (μM) |
| DGKα | 7.5 | 140 | 80 |
| DGKδ | 10 | 140 | 80 |
| DGKζ | 0.5 | 140 | 80 |

The inhibition activities testing for the compound disclosed herein were carried out at room temperature in assay buffer containing 50 mM HEPES, 10 mM MgCl₂, 0.01% BSA, 0.1 mM Na₃VO₄, 0.005% Tween-20 and 0.01 mM CaCl₂. Compounds in DMSO were dispensed into wells of a black 384 well plate (Corning 4514) using D300e digital dispenser (Tecan). The ranges of compounds final concentration were 1.55˜10000 nM or 23.3˜150000 nM. 3 μL 2× enzyme solution was added to wells. After incubation for 1 hour, 3 μL 2× substates solution containing 160 μM DAG and 280 μM ATP was added to the wells to initiate reaction. After 1 hour reaction, 5 μL ADP-Glo reagent (Promga V9101) was added and incubated for 40 minutes. 10 μL Kinase Detection reagent was added and incubated for 30 minutes. Luminescence was measured on a microplate reader (PHERAstar FSX, BMG labtech). The IC₅₀s are calculated based on inhibition of enzyme activity in the presence of increasing concentrations of compounds. Selected compounds had no inhibitory activity on DGKδ. IC₅₀s of the compounds disclosed herein for DGKζ and DGKα are shown in Table 1.

### Baculovirus Expression of Human DGKζ

Human His-TEV-DGK-zeta-pFastBac1 and human baculovirus samples was generated using the Bac-to-Bac baculovirus expression system (Invitrogen) according to the manufacturer's protocol. The DNA used for expression of DGK-zeta, have SEQ ID Nos: 1. Baculovirus amplification was achieved using infected SF9 cells at 1:2000 virus/cells ratios, and grown for 96 hours at 27° C. post-transfected.

The expression scaled up for each protein was carried out in the flask 3 L from CORNING. 4 L of 3×10⁶ cells/mL Sf9 cells (Expression System, Invitrogen) grown in SF900™ II SFM insect medium (Expression System) was infected with virus stock at 1:200 virus/cells ratio, and grown for 48 hours at 27° C. post-transfection. The infected cell culture was harvested by centrifugation at 6000 rpm for 15 min 4° C. in a SORVALL LYNX6000 centrifuge. The cell pellets were stored at −80° C.

### Purification of Human DGK-Zeta

Full length human DGKζ produced as described above, was purified from Sf9 baculovirus-infected insect cell paste. The cells were lysed using sonication method, and the lysates were clarified by centrifugation. The clarified lysates were purified to ˜90% homogeneity, using two successive column chromatography steps on an AKTA Purifier system. The two steps column chromatography included nickel affinity resin capture (i.e. Ni-NTA Agarose, Qiagen), followed by size exclusion chromatography (i.e. Hiload16/60 Superdex200 prep grade, GE Healthcare. The protein was delivered and stored at −80° C. The formulation buffers were identical for the protein: 25 mM Tris, 150 mmol/L NaCl, 2 mM DTT, pH8.0.

SEQ ID No: 1 is Nucleotide sequence encoding
His-TEV-hDGKζ-(M1-V929):
ATGCATCACCATCACCATCACCATCACGAGAACCTGTACTTCCAGGGA
TCCATGGAACCCCGTGATGGTAGCCCCGAAGCTCGTAGCTCCGATTCC
GAGTCCGCCAGCGCTTCCTCCTCCGGTAGCGAACGTGACGCTGGTCCC
GAGCCCGACAAAGCTCCCCGTCGTCTGAATAAGCGCCGTTTTCCCGGT
CTCCGTCTGTTCGGCCACCGCAAGGCCATCACTAAGTCCGGTCTCCAG
CATCTGGCTCCTCCTCCTCCTACCCCCGGTGCTCCTTGCTCCGAATCC
GAGCGCCAGATTCGCTCCACTGTGGATTGGTCCGAAAGCGCCACCTAT
GGTGAGCATATCTGGTTCGAGACCAACGTCTCCGGCGACTTCTGTTAT
GTCGGTGAGCAATACTGTGTGGCTCGTATGCTGCAGAAGTCCGTGTCC
CGCCGTAAATGCGCCGCTTGCAAAATCGTGGTCCATACCCCTTGCATC
GAGCAACTGGAGAAAATCAACTTCCGCTGCAAGCCCAGCTTTCGTGAG
TCCGGTTCCCGCAACGTGCGCGAACCTACTTTCGTGCGCCACCACTGG
GTGCATCGTCGTCGCCAAGACGGCAAATGCCGCCACTGCGGCAAAGGT
TTTCAGCAGAAATTCACCTTCCACAGCAAGGAGATCGTCGCCATCAGC
TGCAGCTGGTGCAAACAAGCTTACCATTCCAAAGTGAGCTGCTTCATG
CTCCAGCAGATCGAAGAGCCTTGCTCTCTGGGTGTGCATGCTGCTGTC
GTGATTCCCCCTACTTGGATTCTGCGTGCTCGCCGTCCCCAGAACACT
CTGAAGGCCTCCAAAAAGAAGAAGCGCGCCAGCTTCAAGCGTAAGAGC
TCCAAAAAGGGTCCCGAAGAGGGCCGTTGGCGTCCCTTCATCATCCGC
CCTACTCCTTCCCCTCTGATGAAGCCTCTGCTGGTCTTCGTCAACCCT
AAGAGCGGCGGCAACCAAGGTGCTAAAATCATCCAGTCCTTCCTCTGG
TATCTGAACCCTCGTCAAGTGTTCGACCTCAGCCAAGGCGGTCCTAAG
GAGGCTCTGGAGATGTACCGCAAGGTCCACAATCTGCGCATCCTCGCT
TGTGGTGGCGATGGCACCGTGGGCTGGATTCTGTCCACTCTGGACCAA
CTGCGTCTGAAACCTCCCCCCCCCGTGGCTATTCTGCCTCTCGGTACC
GGCAACGATCTGGCTCGTACTCTGAATTGGGGTGGTGGCTACACCGAT
GAGCCCGTGTCCAAGATTCTGTCCCACGTCGAAGAAGGCAATGTCGTC
CAACTGGACCGTTGGGACCTCCACGCCGAACCCAACCCCGAGGCTGGC
CCCGAGGACCGTGACGAGGGCGCTACTGACCGTCTGCCCCTCGACGTC
TTCAATAATTACTTCTCTCTGGGCTTTGACGCTCACGTGACTCTGGAA
TTTCATGAAAGCCGCGAGGCCAACCCCGAGAAGTTCAATTCCCGTTTC
CGCAACAAGATGTTCTACGCTGGCACCGCCTTCAGCGACTTCCTCATG
GGCTCCAGCAAGGACCTCGCTAAGCATATCCGCGTGGTGTGCGATGGC
ATGGATCTGACCCCTAAGATCCAAGATCTGAAGCCCCAATGTGTCGTG
TTTCTGAACATCCCCCGCTACTGCGCTGGTACTATGCCTTGGGGCCAT
CCCGGTGAACACCATGACTTCGAACCTCAGCGTCATGATGACGGCTAT
CTGGAGGTGATCGGTTTCACCATGACCTCCCTCGCTGCTCTGCAAGTG
GGTGGCCACGGCGAACGTCTGACTCAATGCCGCGAGGTGGTGCTGACC
ACCAGCAAAGCCATCCCCGTCCAAGTGGATGGTGAGCCTTGCAAGCTG
GCCGCCTCCCGTATCCGTATCGCTCTCCGCAATCAAGCTACCATGGTC
CAGAAGGCCAAACGCCGCAGCGCTGCTCCTCTCCACAGCGACCAACAA
CCCGTCCCCGAACAGCTGCGCATCCAAGTGTCCCGTGTCAGCATGCAT
GACTACGAGGCTCTGCACTACGACAAGGAACAGCTGAAGGAAGCCAGC
GTGCCTCTGGGTACTGTGGTCGTGCCCGGTGACAGCGATCTGGAGCTC
TGCCGTGCCCACATCGAGCGTCTGCAGCAAGAGCCCGACGGTGCTGGT
GCCAAGAGCCCTACTTGCCAAAAACTCTCCCCCAAGTGGTGTTTCCTC
GACGCTACCACCGCCAGCCGCTTCTACCGCATTGATCGCGCCCAAGAG
CATCTGAACTATGTCACCGAGATCGCTCAAGACGAGATCTACATCCTC
GACCCCGAACTCCTCGGTGCTAGCGCCCGTCCCGACCTCCCCACTCCT
ACCTCCCCTCTGCCCACTTCCCCTTGTTCCCCCACCCCTCGTAGCCTC
CAAGGTGATGCTGCCCCTCCTCAAGGTGAGGAGCTCATTGAGGCCGCT
AAGCGTAACGATTTCTGCAAGCTCCAAGAGCTGCATCGTGCTGGTGGC
GACCTCATGCACCGCGATGAGCAGAGCCGCACTCTGCTGCACCACGCT
GTGTCCACTGGTAGCAAGGACGTGGTGCGCTATCTGCTGGACCACGCT
CCTCCCGAGATCCTCGACGCTGTGGAAGAAAACGGCGAGACTTGCCTC
CACCAAGCTGCTGCTCTGGGTCAACGTACCATCTGCCACTACATCGTC
GAAGCTGGTGCTTCTCTGATGAAGACCGACCAGCAAGGTGATACTCCC
CGTCAGCGCGCCGAGAAAGCCCAAGACACCGAACTGGCTGCCTATCTG
GAGAACCGTCAGCACTACCAGATGATTCAGCGTGAAGACCAAGAGACC
GCCGTGTAA
SEQ ID No: 2 is Amino acid sequence of
His-TEV-hDGKζ-(M1-V929):
MEPRDGSPEARSSDSESASASSSGSERDAGPEPDKAPRRLNKRRFPGL
RLFGHRKAITKSGLQHLAPPPPTPGAPCSESERQIRSTVDWSESATYG
EHIWFETNVSGDFCYVGEQYCVARMLQKSVSRRKCAACKIVVHTPCIE
QLEKINFRCKPSFRESGSRNVREPTFVRHHWVHRRRQDGKCRHCGKGF
QQKFTFHSKEIVAISCSWCKQAYHSKVSCFMLQQIEEPCSLGVHAAVV
IPPTWILRARRPQNTLKASKKKKRASFKRKSSKKGPEEGRWRPFIIRP
TPSPLMKPLLVFVNPKSGGNQGAKIIQSFLWYLNPRQVFDLSQGGPKE
ALEMYRKVHNLRILACGGDGTVGWILSTLDQLRLKPPPPVAILPLGTG
NDLARTLNWGGGYTDEPVSKILSHVEEGNVVQLDRWDLHAEPNPEAGP
EDRDEGATDRLPLDVFNNYFSLGFDAHVTLEFHESREANPEKFNSRFR
NKMFYAGTAFSDFLMGSSKDLAKHIRVVCDGMDLTPKIQDLKPQCVVF
LNIPRYCAGTMPWGHPGEHHDFEPQRHDDGYLEVIGFTMTSLAALQVG
GHGERLTQCREVVLTTSKAIPVQVDGEPCKLAASRIRIALRNQATMVQ
KAKRRSAAPLHSDQQPVPEQLRIQVSRVSMHDYEALHYDKEQLKEASV
PLGTVVVPGDSDLELCRAHIERLQQEPDGAGAKSPTCQKLSPKWCFLD
ATTASRFYRIDRAQEHLNYVTEIAQDEIYILDPELLGASARPDLPTPT
SPLPTSPCSPTPRSLQGDAAPPQGEELIEAAKRNDFCKLQELHRAGGD
LMHRDEQSRTLLHHAVSTGSKDVVRYLLDHAPPEILDAVEENGETCLH
QAAALGQRTICHYIVEAGASLMKTDQQGDTPRQRAEKAQDTELAAYLE
NRQHYQMIQREDQETAV*

TABLE 1

| Compound No. | Compound structure | IC₅₀s for DGKζ | IC₅₀s for DGKα |
|---|---|---|---|
| A1 | 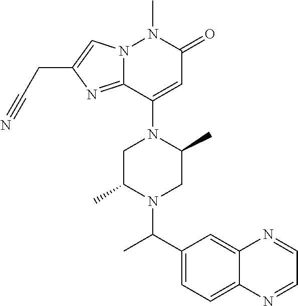 [SMILES: CC(c1ccc2nccnc2c1)N1C[C@H](C)N(c2cc(=O)n(C)n3cc(CC#N)nc23)C[C@H]1C] | 78 | 2,000 |
| A1a | 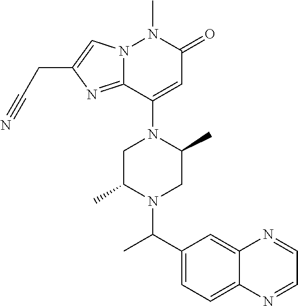 [SMILES: CC(c1ccc2nccnc2c1)N1C[C@H](C)N(c2cc(=O)n(C)n3cc(CC#N)nc23)C[C@H]1C] | 35 | 1,270 |
| A1b | 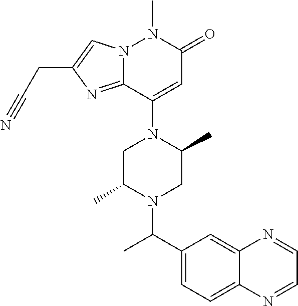 [SMILES: CC(c1ccc2nccnc2c1)N1C[C@H](C)N(c2cc(=O)n(C)n3cc(CC#N)nc23)C[C@H]1C] | >10,000 | 2,750 |
| A2a | 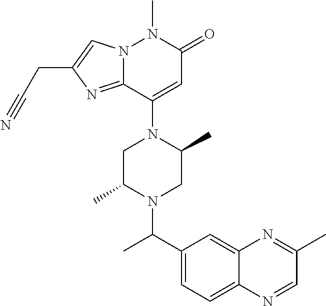 [SMILES: Cc1cnc2ccc(C(C)N3C[C@H](C)N(c4cc(=O)n(C)n5cc(CC#N)nc45)C[C@H]3C)cc2n1] | 7,400 | 26,800 |
| A2b | 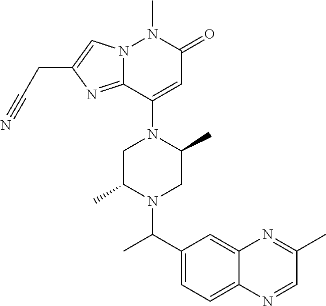 [SMILES: Cc1cnc2ccc(C(C)N3C[C@H](C)N(c4cc(=O)n(C)n5cc(CC#N)nc45)C[C@H]3C)cc2n1] | 11 | 11,600 |
| A3 | 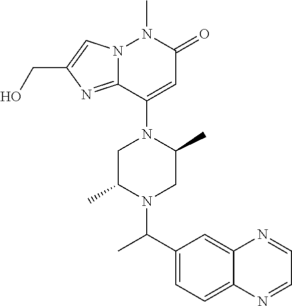 [SMILES: CC(c1ccc2nccnc2c1)N1C[C@H](C)N(c2cc(=O)n(C)n3cc(CO)nc23)C[C@H]1C] | 9,000 | 2,300 |
| A4 | 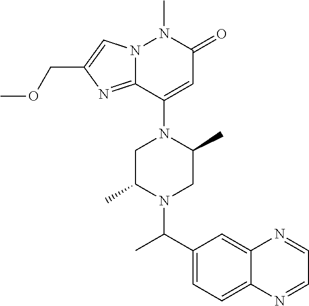 [SMILES: COCc1cn2c(n1)c(N1C[C@@H](C)N(C(C)c3ccc4nccnc4c3)C[C@@H]1C)cc(=O)n2C] | 480 | 4,000 |
| A5 | 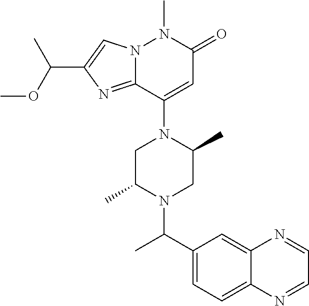 [SMILES: COC(C)c1cn2c(n1)c(N1C[C@@H](C)N(C(C)c3ccc4nccnc4c3)C[C@@H]1C)cc(=O)n2C] | 11,000 | 22,000 |
| A6 | 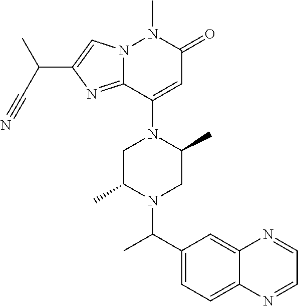 [SMILES: CC(C#N)c1cn2c(n1)c(N1C[C@@H](C)N(C(C)c3ccc4nccnc4c3)C[C@@H]1C)cc(=O)n2C] | >10,000 | >10,000 |
| A7 | 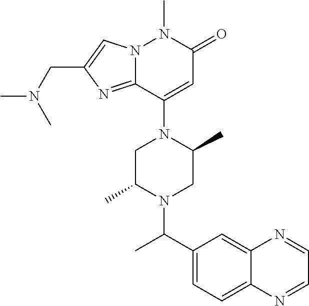 [SMILES: CC(c1ccc2nccnc2c1)N1C[C@H](C)N(c2cc(=O)n(C)n3cc(CN(C)C)nc23)C[C@H]1C] | 5,470 | >10,000 |
| A8a | 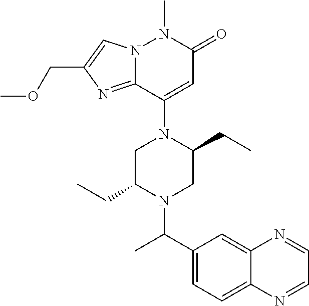 [SMILES: CC[C@H]1CN(C(C)c2ccc3nccnc3c2)[C@H](CC)CN1c1cc(=O)n(C)n2cc(COC)nc12] | 25 | 1,310 |
| A8b | 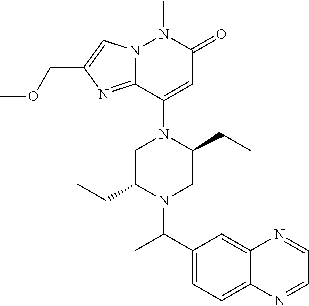 [SMILES: CC[C@H]1CN(C(C)c2ccc3nccnc3c2)[C@H](CC)CN1c1cc(=O)n(C)n2cc(COC)nc12] | 1,330 | 5,080 |
| A9a | 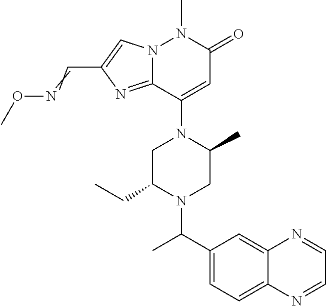 [SMILES: CC[C@@H]1CN(c2cc(=O)n(C)n3cc(C=NOC)nc23)[C@@H](C)CN1C(C)c1ccc2nccnc2c1] | 3,150 | 739 |
| A9b | 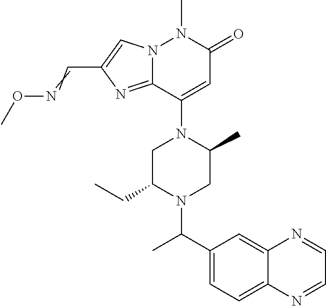 [SMILES: CC[C@@H]1CN(c2cc(=O)n(C)n3cc(C=NOC)nc23)[C@@H](C)CN1C(C)c1ccc2nccnc2c1] | 313 | 74 |
| A9c | 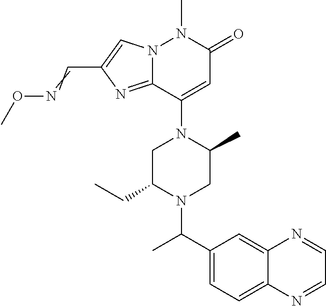 [SMILES: CC[C@@H]1CN(c2cc(=O)n(C)n3cc(C=NOC)nc23)[C@@H](C)CN1C(C)c1ccc2nccnc2c1] | >10,000 | 2,170 |
| A9d | 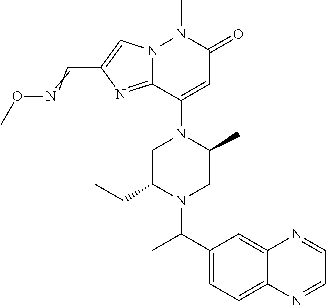 [SMILES: CC[C@@H]1CN(c2cc(=O)n(C)n3cc(C=NOC)nc23)[C@@H](C)CN1C(C)c1ccc2nccnc2c1] | >10,000 | 211 |
| A10 | 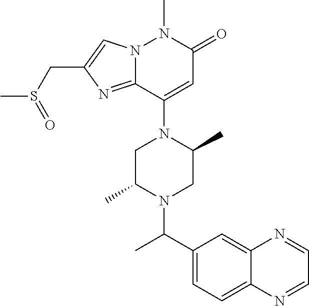 [SMILES: CC(c1ccc2nccnc2c1)N1C[C@H](C)N(c2cc(=O)n(C)n3cc(CS(C)=O)nc23)C[C@H]1C] | >10,000 | >10,000 |
| A11 | 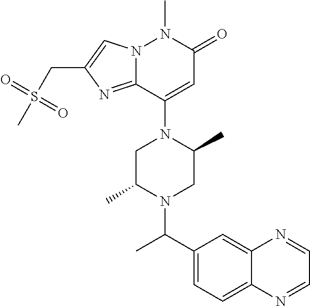 [SMILES: CC(c1ccc2nccnc2c1)N1C[C@H](C)N(c2cc(=O)n(C)n3cc(CS(C)(=O)=O)nc23)C[C@H]1C] | >10,000 | >10,000 |
| A12 | 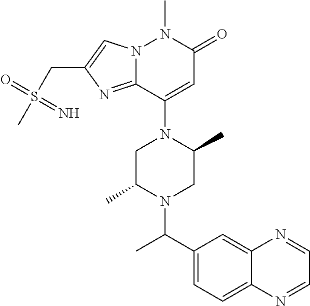 [SMILES: CC(c1ccc2nccnc2c1)N1C[C@H](C)N(c2cc(=O)n(C)n3cc(CS(C)(=N)=O)nc23)C[C@H]1C] | >10,000 | >10,000 |
| A13a | 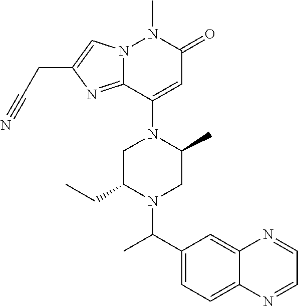 [SMILES: CC[C@@H]1CN(c2cc(=O)n(C)n3cc(CC#N)nc23)[C@@H](C)CN1C(C)c1ccc2nccnc2c1] | 16 | 966 |
| A13b | 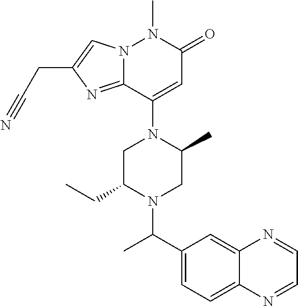 [SMILES: CC[C@@H]1CN(c2cc(=O)n(C)n3cc(CC#N)nc23)[C@@H](C)CN1C(C)c1ccc2nccnc2c1] | 3,610 | 4,180 |
| A14a | 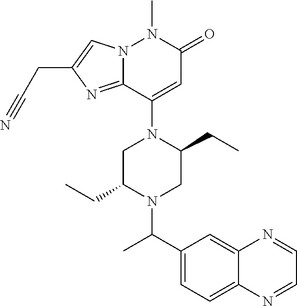 [SMILES: CC[C@H]1CN(C(C)c2ccc3nccnc3c2)[C@H](CC)CN1c1cc(=O)n(C)n2cc(CC#N)nc12] | 6 | 217 |
| A14b | 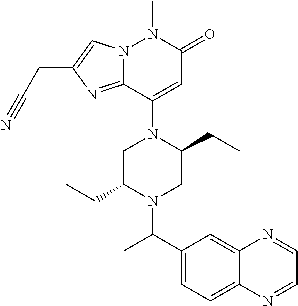 [SMILES: CC[C@H]1CN(C(C)c2ccc3nccnc3c2)[C@H](CC)CN1c1cc(=O)n(C)n2cc(CC#N)nc12] | 358 | 1,420 |
| A15 | 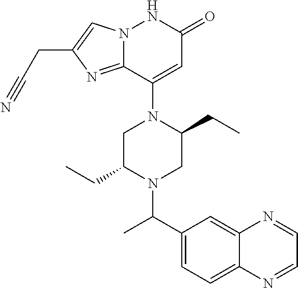 [SMILES: CC[C@H]1CN(C(C)c2ccc3nccnc3c2)[C@H](CC)CN1c1cc(=O)[nH]n2cc(CC#N)nc12] | 3,870 | 4,380 |
| A16a | 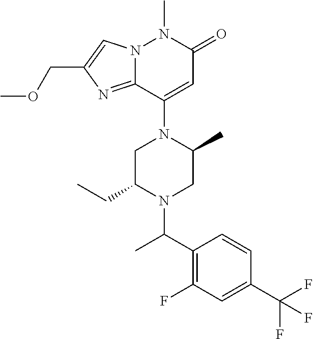 [SMILES: CC[C@@H]1CN(c2cc(=O)n(C)n3cc(COC)nc23)[C@@H](C)CN1C(C)c1ccc(C(F)(F)F)cc1F] | 1,230 | 4,650 |
| A16b | 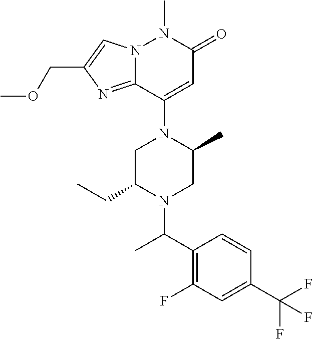 [SMILES: CC[C@@H]1CN(c2cc(=O)n(C)n3cc(COC)nc23)[C@@H](C)CN1C(C)c1ccc(C(F)(F)F)cc1F] | >10,000 | 8,460 |
| A17a | 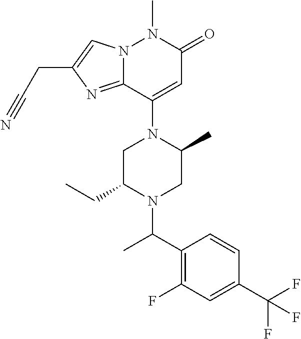 [SMILES: CC[C@@H]1CN(c2cc(=O)n(C)n3cc(CC#N)nc23)[C@@H](C)CN1C(C)c1ccc(C(F)(F)F)cc1F] | 108 | 171 |
| A17b | 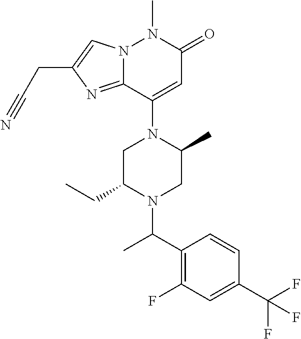 [SMILES: CC[C@@H]1CN(c2cc(=O)n(C)n3cc(CC#N)nc23)[C@@H](C)CN1C(C)c1ccc(C(F)(F)F)cc1F] | 4,720 | 823 |
| A18 | 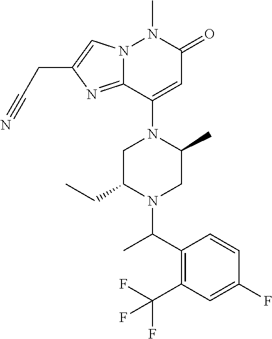 [SMILES: CC[C@@H]1CN(c2cc(=O)n(C)n3cc(CC#N)nc23)[C@@H](C)CN1C(C)c1ccc(F)cc1C(F)(F)F] | 98 | 17,700 |
| A19 | 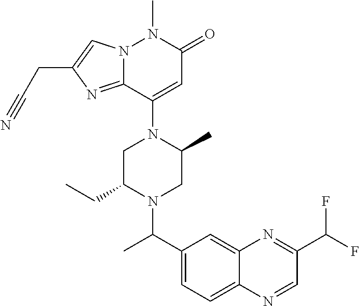 [SMILES: CC[C@@H]1CN(c2cc(=O)n(C)n3cc(CC#N)nc23)[C@@H](C)CN1C(C)c1ccc2ncc(C(F)F)nc2c1] | 19 | 2,380 |
| A20 | 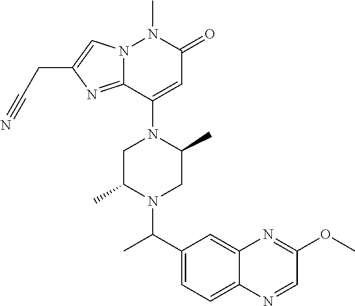 [SMILES: COc1cnc2ccc(C(C)N3C[C@H](C)N(c4cc(=O)n(C)n5cc(CC#N)nc45)C[C@H]3C)cc2n1] | 18 | 1,020 |
| A21 | 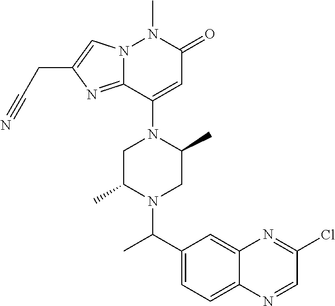 [SMILES: CC(c1ccc2ncc(Cl)nc2c1)N1C[C@H](C)N(c2cc(=O)n(C)n3cc(CC#N)nc23)C[C@H]1C] | 27 | 2,710 |
| A22 | 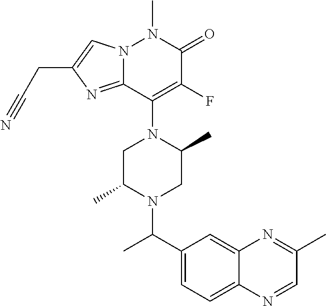 [SMILES: Cc1cnc2ccc(C(C)N3C[C@H](C)N(c4c(F)c(=O)n(C)n5cc(CC#N)nc45)C[C@H]3C)cc2n1] | 95 | 10,800 |
| A23 | 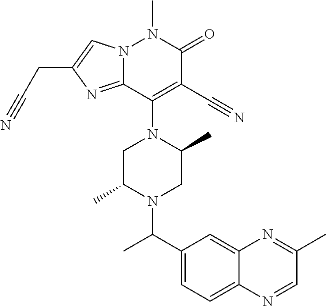 [SMILES: Cc1cnc2ccc(C(C)N3C[C@H](C)N(c4c(C#N)c(=O)n(C)n5cc(CC#N)nc45)C[C@H]3C)cc2n1] | 39 | 2,280 |
| A24 | 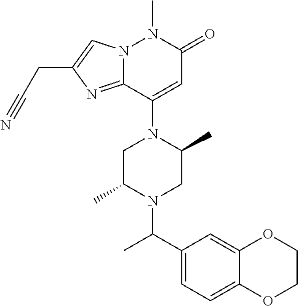 [SMILES: CC(c1ccc2c(c1)OCCO2)N1C[C@H](C)N(c2cc(=O)n(C)n3cc(CC#N)nc23)C[C@H]1C] | 230 | 2,870 |
| A25 | 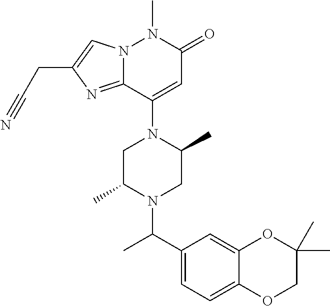 [SMILES: CC(c1ccc2c(c1)OC(C)(C)CO2)N1C[C@H](C)N(c2cc(=O)n(C)n3cc(CC#N)nc23)C[C@H]1C] | 9 | 1,660 |
| A25a | 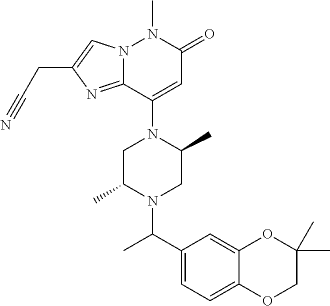 [SMILES: CC(c1ccc2c(c1)OC(C)(C)CO2)N1C[C@H](C)N(c2cc(=O)n(C)n3cc(CC#N)nc23)C[C@H]1C] | 5 | 2,500 |
| A25b | 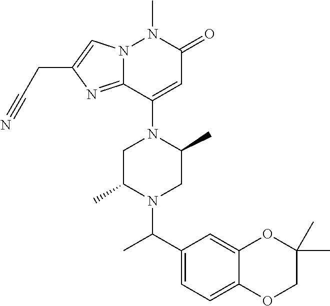 [SMILES: CC(c1ccc2c(c1)OC(C)(C)CO2)N1C[C@H](C)N(c2cc(=O)n(C)n3cc(CC#N)nc23)C[C@H]1C] | 3,970 | 1,030 |
| A26 | 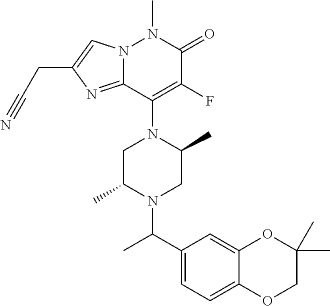 [SMILES: CC(c1ccc2c(c1)OC(C)(C)CO2)N1C[C@H](C)N(c2c(F)c(=O)n(C)n3cc(CC#N)nc23)C[C@H]1C] | 16 | 3,090 |
| A26a | 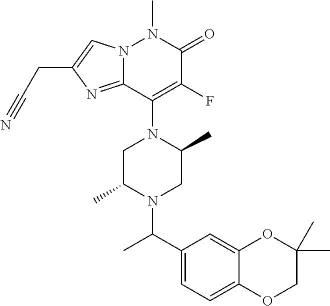 [SMILES: CC(c1ccc2c(c1)OC(C)(C)CO2)N1C[C@H](C)N(c2c(F)c(=O)n(C)n3cc(CC#N)nc23)C[C@H]1C] | 19 | 2,890 |
| A26b | 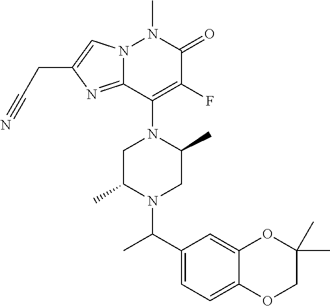 [SMILES: CC(c1ccc2c(c1)OC(C)(C)CO2)N1C[C@H](C)N(c2c(F)c(=O)n(C)n3cc(CC#N)nc23)C[C@H]1C] | 2,140 | 2,110 |
| A27 | 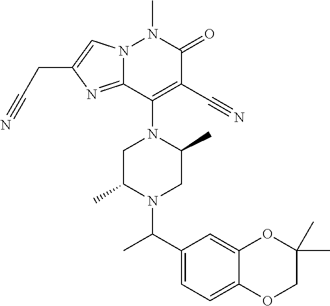 [SMILES: CC(c1ccc2c(c1)OC(C)(C)CO2)N1C[C@H](C)N(c2c(C#N)c(=O)n(C)n3cc(CC#N)nc23)C[C@H]1C] | 10 | 858 |
| A27a | 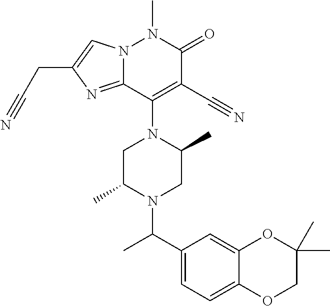 [SMILES: CC(c1ccc2c(c1)OC(C)(C)CO2)N1C[C@H](C)N(c2c(C#N)c(=O)n(C)n3cc(CC#N)nc23)C[C@H]1C] | 3 | 2,250 |
| A27b | 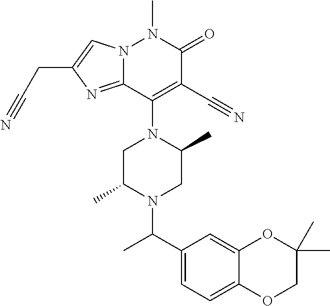 [SMILES: CC(c1ccc2c(c1)OC(C)(C)CO2)N1C[C@H](C)N(c2c(C#N)c(=O)n(C)n3cc(CC#N)nc23)C[C@H]1C] | 579 | 486 |
| A28 | 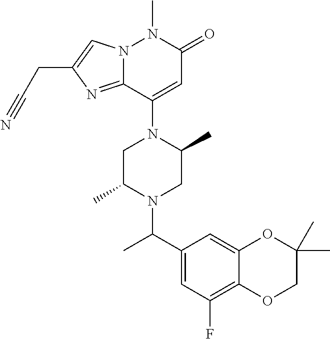 [SMILES: CC(c1cc(F)c2c(c1)OC(C)(C)CO2)N1C[C@H](C)N(c2cc(=O)n(C)n3cc(CC#N)nc23)C[C@H]1C] | 69 | 18,100 |
| A29 | 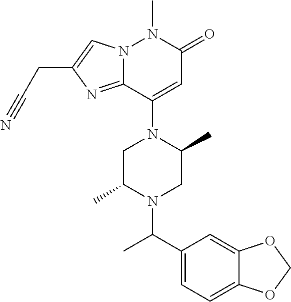 [SMILES: CC(c1ccc2c(c1)OCO2)N1C[C@H](C)N(c2cc(=O)n(C)n3cc(CC#N)nc23)C[C@H]1C] | 474 | 296 |
| A30 | 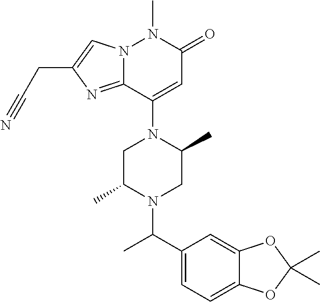 [SMILES: CC(c1ccc2c(c1)OC(C)(C)O2)N1C[C@H](C)N(c2cc(=O)n(C)n3cc(CC#N)nc23)C[C@H]1C] | 100 | 3,950 |
| A31 | 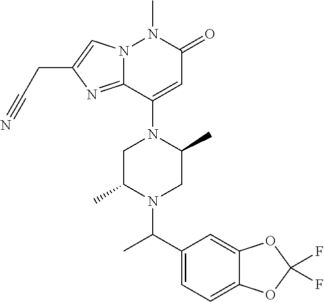 [SMILES: CC(c1ccc2c(c1)OC(F)(F)O2)N1C[C@H](C)N(c2cc(=O)n(C)n3cc(CC#N)nc23)C[C@H]1C] | 1,130 | 1,970 |
| A32 | 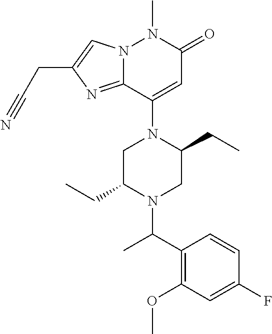 [SMILES: CC[C@H]1CN(C(C)c2ccc(F)cc2OC)[C@H](CC)CN1c1cc(=O)n(C)n2cc(CC#N)nc12] | 110 | 6,420 |
| A33 | 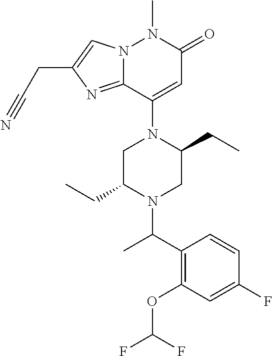 [SMILES: CC[C@H]1CN(C(C)c2ccc(F)cc2OC(F)F)[C@H](CC)CN1c1cc(=O)n(C)n2cc(CC#N)nc12] | 85 | 8,800 |
| A34 | 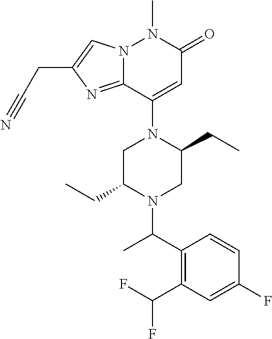 [SMILES: CC[C@H]1CN(C(C)c2ccc(F)cc2C(F)F)[C@H](CC)CN1c1cc(=O)n(C)n2cc(CC#N)nc12] | 14 | 3,510 |
| A35 | 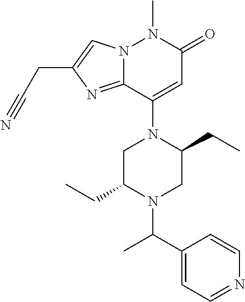 [SMILES: CC[C@H]1CN(C(C)c2ccncc2)[C@H](CC)CN1c1cc(=O)n(C)n2cc(CC#N)nc12] | 3,120 | 42 |
| A36 | 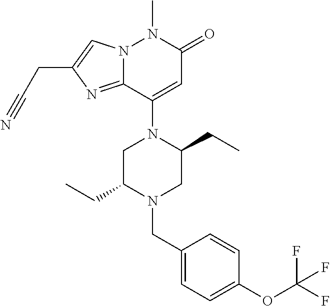 [SMILES: CC[C@@H]1CN(c2cc(=O)n(C)n3cc(CC#N)nc23)[C@@H](CC)CN1Cc1ccc(OC(F)(F)F)cc1] | 241 | 14 |
| A37a | 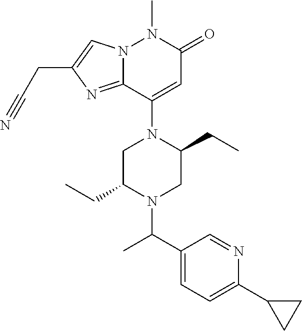 [SMILES: CC[C@H]1CN(C(C)c2ccc(C3CC3)nc2)[C@H](CC)CN1c1cc(=O)n(C)n2cc(CC#N)nc12] | 20 | 44 |
| A37b | 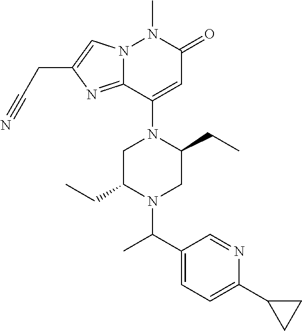 [SMILES: CC[C@H]1CN(C(C)c2ccc(C3CC3)nc2)[C@H](CC)CN1c1cc(=O)n(C)n2cc(CC#N)nc12] | 538 | 260 |
| A38a | 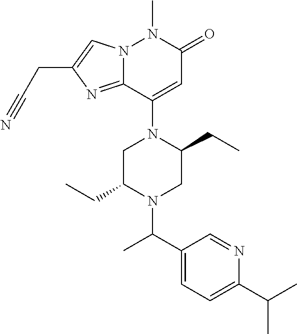 [SMILES: CC[C@H]1CN(C(C)c2ccc(C(C)C)nc2)[C@H](CC)CN1c1cc(=O)n(C)n2cc(CC#N)nc12] | 21 | 43 |
| A38b | 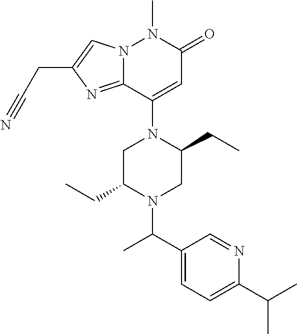 [SMILES: CC[C@H]1CN(C(C)c2ccc(C(C)C)nc2)[C@H](CC)CN1c1cc(=O)n(C)n2cc(CC#N)nc12] | 475 | 423 |
| A39 | 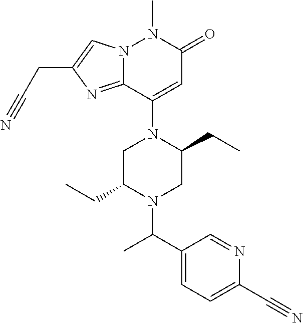 [SMILES: CC[C@H]1CN(C(C)c2ccc(C#N)nc2)[C@H](CC)CN1c1cc(=O)n(C)n2cc(CC#N)nc12] | 1,140 | 276 |
| A40 | 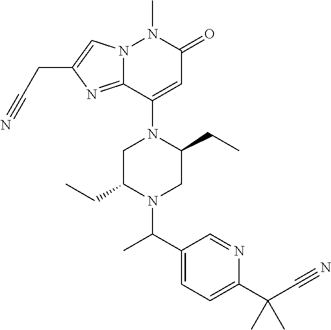 [SMILES: CC[C@H]1CN(C(C)c2ccc(C(C)(C)C#N)nc2)[C@H](CC)CN1c1cc(=O)n(C)n2cc(CC#N)nc12] | 124 | 297 |
| A41 | 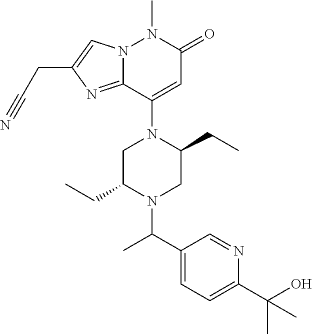 [SMILES: CC[C@H]1CN(C(C)c2ccc(C(C)(C)O)nc2)[C@H](CC)CN1c1cc(=O)n(C)n2cc(CC#N)nc12] | 844 | 1,270 |
| A42a | 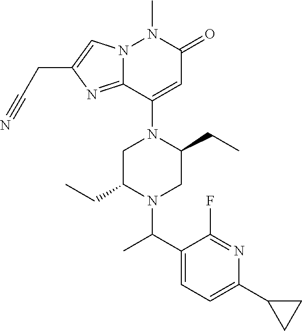 [SMILES: CC[C@H]1CN(C(C)c2ccc(C3CC3)nc2F)[C@H](CC)CN1c1cc(=O)n(C)n2cc(CC#N)nc12] | 57 | 339 |
| A42b | 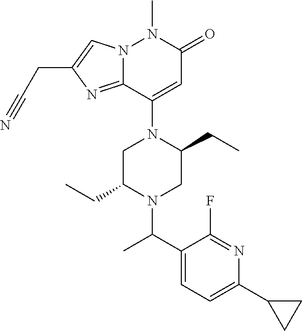 [SMILES: CC[C@H]1CN(C(C)c2ccc(C3CC3)nc2F)[C@H](CC)CN1c1cc(=O)n(C)n2cc(CC#N)nc12] | 1,250 | 1,060 |
| A43a | 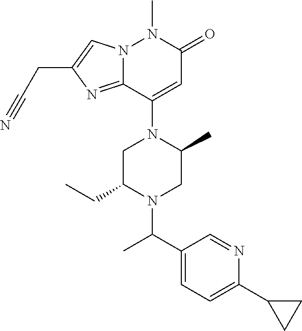 [SMILES: CC[C@@H]1CN(c2cc(=O)n(C)n3cc(CC#N)nc23)[C@@H](C)CN1C(C)c1ccc(C2CC2)nc1] | 202 | 285 |
| A43b | 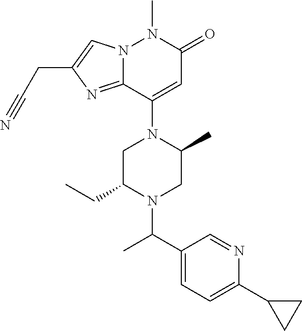 [SMILES: CC[C@@H]1CN(c2cc(=O)n(C)n3cc(CC#N)nc23)[C@@H](C)CN1C(C)c1ccc(C2CC2)nc1] | >10,000 | 1,490 |
| A44a | 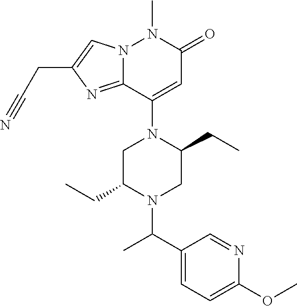 [SMILES: CC[C@H]1CN(C(C)c2ccc(OC)nc2)[C@H](CC)CN1c1cc(=O)n(C)n2cc(CC#N)nc12] | 52 | 96 |
| A44b | 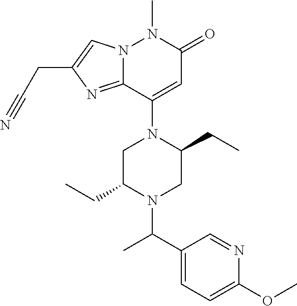 [SMILES: CC[C@H]1CN(C(C)c2ccc(OC)nc2)[C@H](CC)CN1c1cc(=O)n(C)n2cc(CC#N)nc12] | 1,280 | 508 |
| A45a | 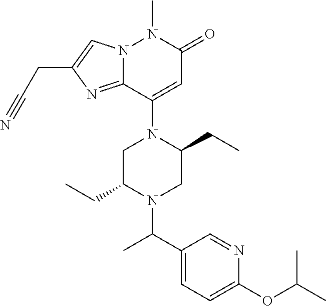 [SMILES: CC[C@H]1CN(C(C)c2ccc(OC(C)C)nc2)[C@H](CC)CN1c1cc(=O)n(C)n2cc(CC#N)nc12] | 8.0 | 941 |
| A45b | 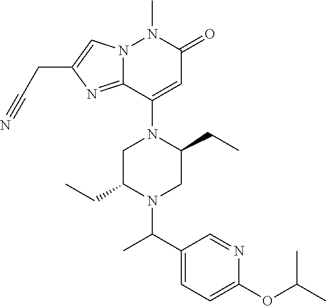 [SMILES: CC[C@H]1CN(C(C)c2ccc(OC(C)C)nc2)[C@H](CC)CN1c1cc(=O)n(C)n2cc(CC#N)nc12] | 1,060 | 835 |
| A46a | 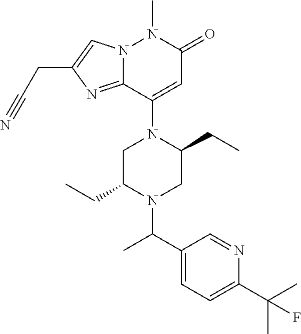 [SMILES: CC[C@H]1CN(C(C)c2ccc(C(C)(C)F)nc2)[C@H](CC)CN1c1cc(=O)n(C)n2cc(CC#N)nc12] | 6.4 | 122 |
| A46b | 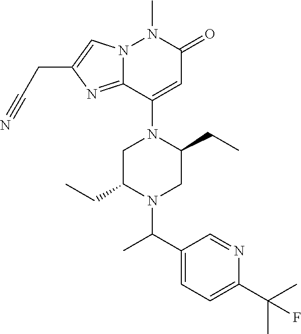 [SMILES: CC[C@H]1CN(C(C)c2ccc(C(C)(C)F)nc2)[C@H](CC)CN1c1cc(=O)n(C)n2cc(CC#N)nc12] | 877 | 671 |
| A47a | 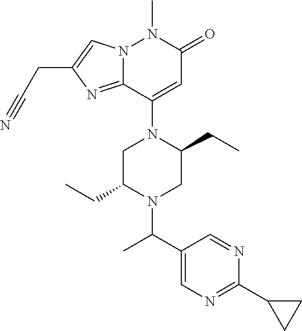 [SMILES: CC[C@H]1CN(C(C)c2cnc(C3CC3)nc2)[C@H](CC)CN1c1cc(=O)n(C)n2cc(CC#N)nc12] | 90 | 2,090 |
| A47b | 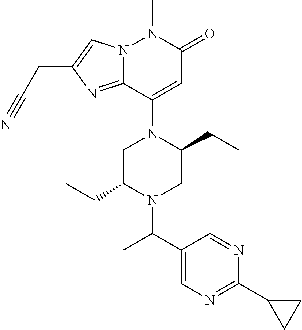 [SMILES: CC[C@H]1CN(C(C)c2cnc(C3CC3)nc2)[C@H](CC)CN1c1cc(=O)n(C)n2cc(CC#N)nc12] | 1,350 | 1,610 |
| A48 | 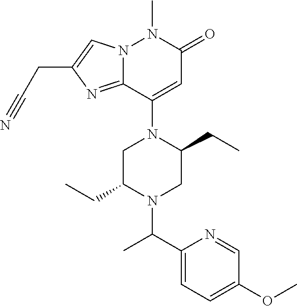 [SMILES: CC[C@H]1CN(C(C)c2ccc(OC)cn2)[C@H](CC)CN1c1cc(=O)n(C)n2cc(CC#N)nc12] | 445 | 30 |
| A49 | 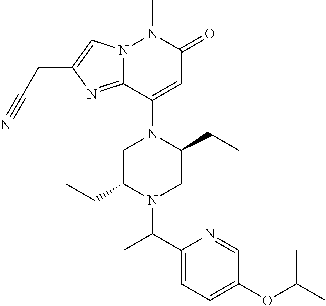 [SMILES: CC[C@H]1CN(C(C)c2ccc(OC(C)C)cn2)[C@H](CC)CN1c1cc(=O)n(C)n2cc(CC#N)nc12] | 128 | 37 |
| A50a | 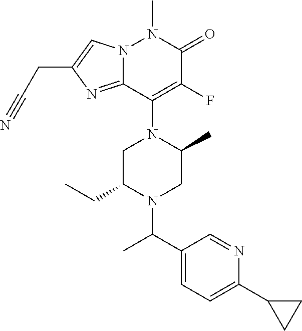 [SMILES: CC[C@@H]1CN(c2c(F)c(=O)n(C)n3cc(CC#N)nc23)[C@@H](C)CN1C(C)c1ccc(C2CC2)nc1] | 708 | 623 |
| A50b | 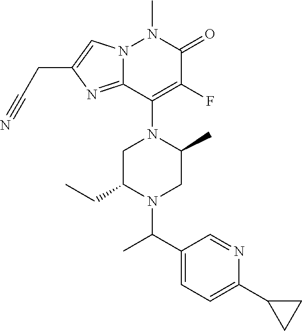 [SMILES: CC[C@@H]1CN(c2c(F)c(=O)n(C)n3cc(CC#N)nc23)[C@@H](C)CN1C(C)c1ccc(C2CC2)nc1] | 2,370 | 1,080 |
| A51 | 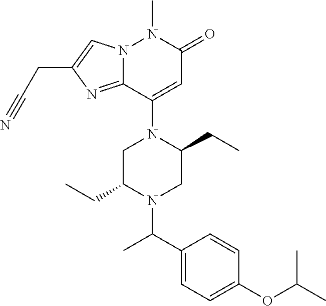 [SMILES: CC[C@H]1CN(C(C)c2ccc(OC(C)C)cc2)[C@H](CC)CN1c1cc(=O)n(C)n2cc(CC#N)nc12] | 284 | 90 |
| A52 | 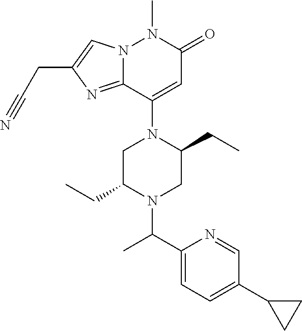 [SMILES: CC[C@H]1CN(C(C)c2ccc(C3CC3)cn2)[C@H](CC)CN1c1cc(=O)n(C)n2cc(CC#N)nc12] | 75 | 18 |
| A53 | 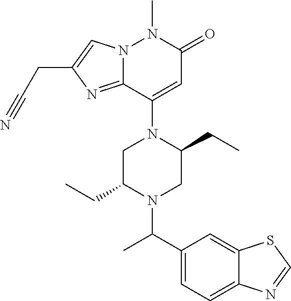 [SMILES: CC[C@H]1CN(C(C)c2ccc3ncsc3c2)[C@H](CC)CN1c1cc(=O)n(C)n2cc(CC#N)nc12] | 6.5 | 24 |
| A53a | 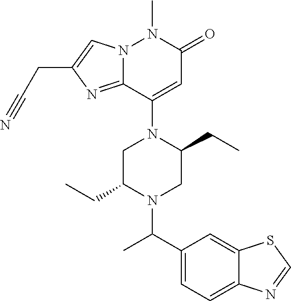 [SMILES: CC[C@H]1CN(C(C)c2ccc3ncsc3c2)[C@H](CC)CN1c1cc(=O)n(C)n2cc(CC#N)nc12] | 4 | 11 |
| A53b | 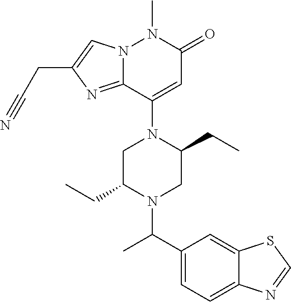 [SMILES: CC[C@H]1CN(C(C)c2ccc3ncsc3c2)[C@H](CC)CN1c1cc(=O)n(C)n2cc(CC#N)nc12] | 215 | 235 |
| A54 | 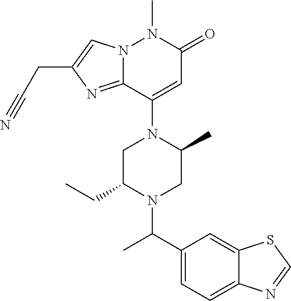 [SMILES: CC[C@@H]1CN(c2cc(=O)n(C)n3cc(CC#N)nc23)[C@@H](C)CN1C(C)c1ccc2ncsc2c1] | 21 | 86 |
| A55 | 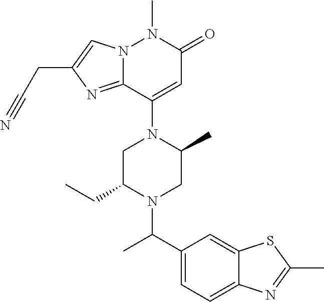 [SMILES: CC[C@@H]1CN(c2cc(=O)n(C)n3cc(CC#N)nc23)[C@@H](C)CN1C(C)c1ccc2nc(C)sc2c1] | 108 | 789 |
| A56a | 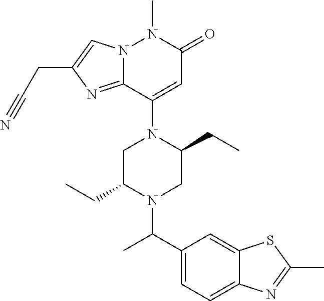 [SMILES: CC[C@H]1CN(C(C)c2ccc3nc(C)sc3c2)[C@H](CC)CN1c1cc(=O)n(C)n2cc(CC#N)nc12] | 11 | 87 |
| A56b | 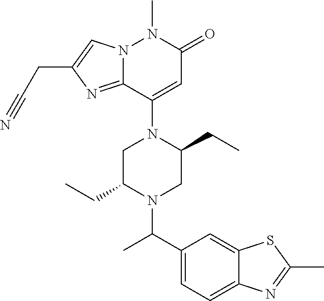 [SMILES: CC[C@H]1CN(C(C)c2ccc3nc(C)sc3c2)[C@H](CC)CN1c1cc(=O)n(C)n2cc(CC#N)nc12] | 638 | 659 |
| A57a | 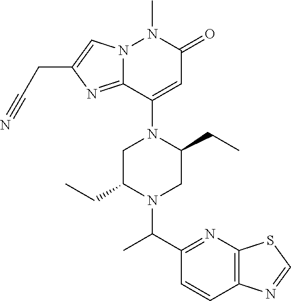 [SMILES: CC[C@H]1CN(C(C)c2ccc3ncsc3n2)[C@H](CC)CN1c1cc(=O)n(C)n2cc(CC#N)nc12] | 6.8 | 7.9 |
| A57b | 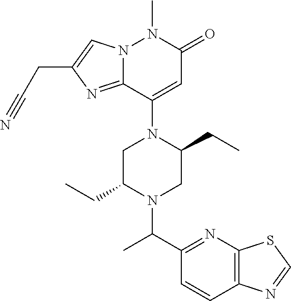 [SMILES: CC[C@H]1CN(C(C)c2ccc3ncsc3n2)[C@H](CC)CN1c1cc(=O)n(C)n2cc(CC#N)nc12] | 357 | 172 |
| A58a | 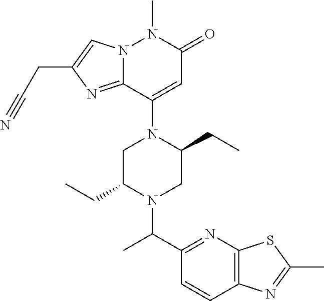 [SMILES: CC[C@H]1CN(C(C)c2ccc3nc(C)sc3n2)[C@H](CC)CN1c1cc(=O)n(C)n2cc(CC#N)nc12] | 2,160 | 1,220 |
| A58b | 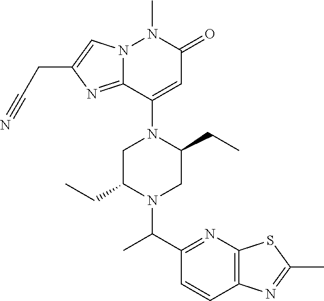 [SMILES: CC[C@H]1CN(C(C)c2ccc3nc(C)sc3n2)[C@H](CC)CN1c1cc(=O)n(C)n2cc(CC#N)nc12] | 7.3 | 13.3 |
| A59a | 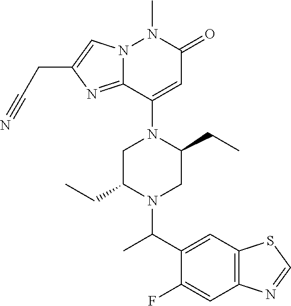 [SMILES: CC[C@H]1CN(C(C)c2cc3scnc3cc2F)[C@H](CC)CN1c1cc(=O)n(C)n2cc(CC#N)nc12] | 26 | 54 |
| A59b | 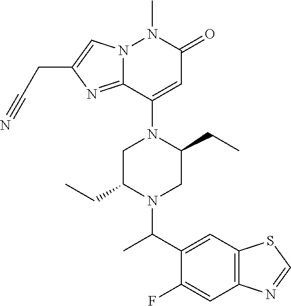 [SMILES: CC[C@H]1CN(C(C)c2cc3scnc3cc2F)[C@H](CC)CN1c1cc(=O)n(C)n2cc(CC#N)nc12] | 2,610 | 1,580 |
| A60a | 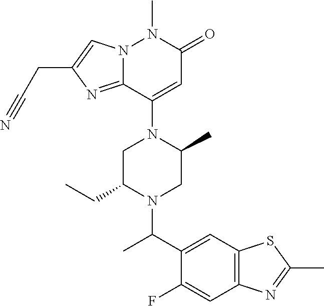 [SMILES: CC[C@@H]1CN(c2cc(=O)n(C)n3cc(CC#N)nc23)[C@@H](C)CN1C(C)c1cc2sc(C)nc2cc1F] | 189 | 826 |
| A60b | 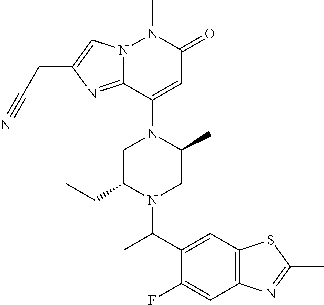 [SMILES: CC[C@@H]1CN(c2cc(=O)n(C)n3cc(CC#N)nc23)[C@@H](C)CN1C(C)c1cc2sc(C)nc2cc1F] | 2,930 | 4,410 |
| A61a | 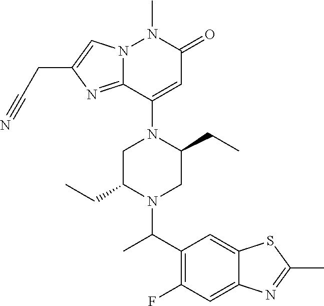 [SMILES: CC[C@H]1CN(C(C)c2cc3sc(C)nc3cc2F)[C@H](CC)CN1c1cc(=O)n(C)n2cc(CC#N)nc12] | 126 | 400 |
| A61b | 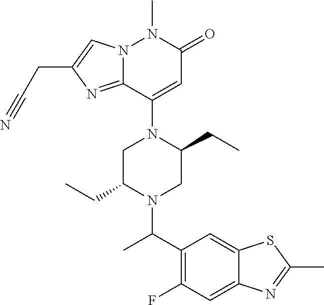 [SMILES: CC[C@H]1CN(C(C)c2cc3sc(C)nc3cc2F)[C@H](CC)CN1c1cc(=O)n(C)n2cc(CC#N)nc12] | 2,000 | 5,520 |
| A62a | 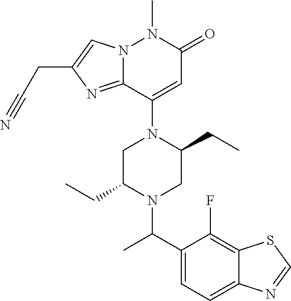 [SMILES: CC[C@H]1CN(C(C)c2ccc3ncsc3c2F)[C@H](CC)CN1c1cc(=O)n(C)n2cc(CC#N)nc12] | 8.5 | 105 |
| A62b | 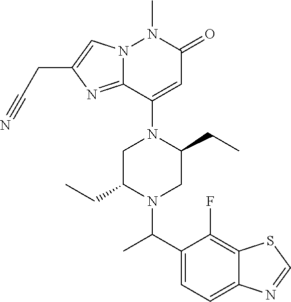 [SMILES: CC[C@H]1CN(C(C)c2ccc3ncsc3c2F)[C@H](CC)CN1c1cc(=O)n(C)n2cc(CC#N)nc12] | 1,210 | 2,390 |
| A63a | 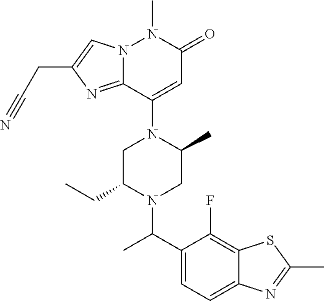 [SMILES: CC[C@@H]1CN(c2cc(=O)n(C)n3cc(CC#N)nc23)[C@@H](C)CN1C(C)c1ccc2nc(C)sc2c1F] | 6.0 | 683 |
| A63b | 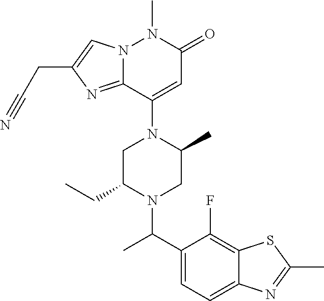 [SMILES: CC[C@@H]1CN(c2cc(=O)n(C)n3cc(CC#N)nc23)[C@@H](C)CN1C(C)c1ccc2nc(C)sc2c1F] | 1,790 | 5,780 |
| A64a | 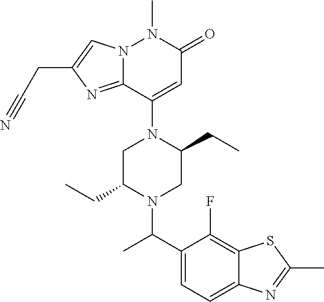 [SMILES: CC[C@H]1CN(C(C)c2ccc3nc(C)sc3c2F)[C@H](CC)CN1c1cc(=O)n(C)n2cc(CC#N)nc12] | 5.7 | 231 |
| A64b | 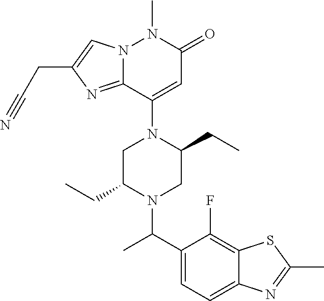 [SMILES: CC[C@H]1CN(C(C)c2ccc3nc(C)sc3c2F)[C@H](CC)CN1c1cc(=O)n(C)n2cc(CC#N)nc12] | 1,420 | 8,150 |
| A65 | 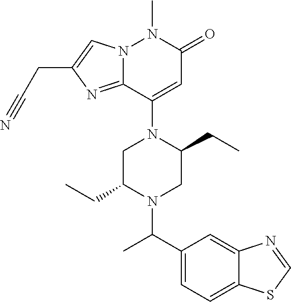 [SMILES: CC[C@H]1CN(C(C)c2ccc3scnc3c2)[C@H](CC)CN1c1cc(=O)n(C)n2cc(CC#N)nc12] | 18 | 36 |
| A65a | 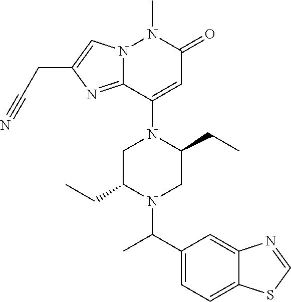 [SMILES: CC[C@H]1CN(C(C)c2ccc3scnc3c2)[C@H](CC)CN1c1cc(=O)n(C)n2cc(CC#N)nc12] | 7 | 47 |
| A65b | 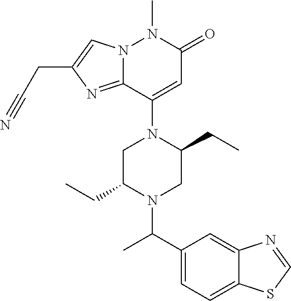 [SMILES: CC[C@H]1CN(C(C)c2ccc3scnc3c2)[C@H](CC)CN1c1cc(=O)n(C)n2cc(CC#N)nc12] | 158 | 157 |
| A66a | 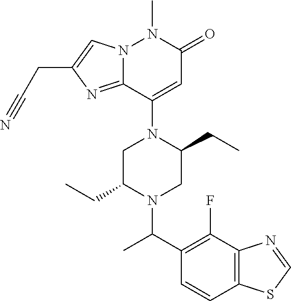 [SMILES: CC[C@H]1CN(C(C)c2ccc3scnc3c2F)[C@H](CC)CN1c1cc(=O)n(C)n2cc(CC#N)nc12] | 22 | 105 |
| A66b | 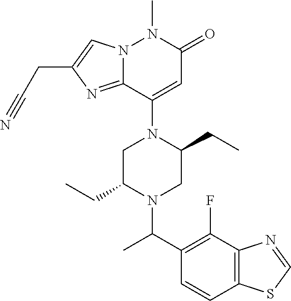 [SMILES: CC[C@H]1CN(C(C)c2ccc3scnc3c2F)[C@H](CC)CN1c1cc(=O)n(C)n2cc(CC#N)nc12] | 1,440 | 1,640 |
| A67a | 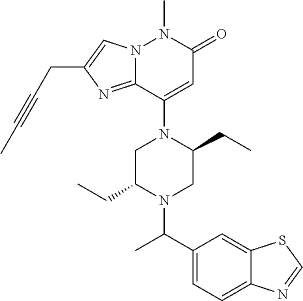 [SMILES: CC#CCc1cn2c(n1)c(N1C[C@@H](CC)N(C(C)c3ccc4ncsc4c3)C[C@@H]1CC)cc(=O)n2C] | 70 | 4 |
| A67b | 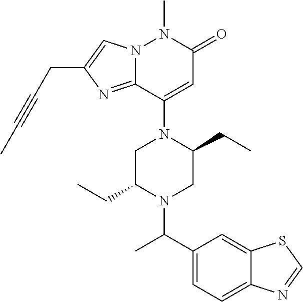 [SMILES: CC#CCc1cn2c(n1)c(N1C[C@@H](CC)N(C(C)c3ccc4ncsc4c3)C[C@@H]1CC)cc(=O)n2C] | 2,360 | 64 |
| A68a | 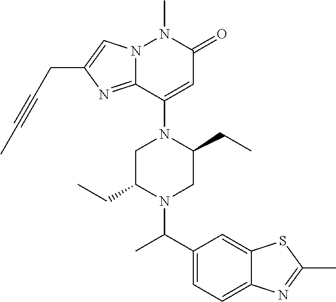 [SMILES: CC#CCc1cn2c(n1)c(N1C[C@@H](CC)N(C(C)c3ccc4nc(C)sc4c3)C[C@@H]1CC)cc(=O)n2C] | 104 | 26 |
| A68b | 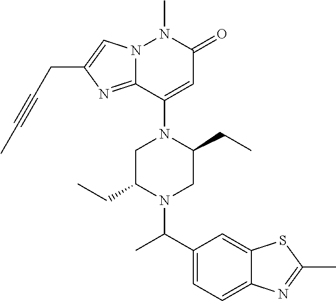 [SMILES: CC#CCc1cn2c(n1)c(N1C[C@@H](CC)N(C(C)c3ccc4nc(C)sc4c3)C[C@@H]1CC)cc(=O)n2C] | >10,000 | 527 |
| A69 | 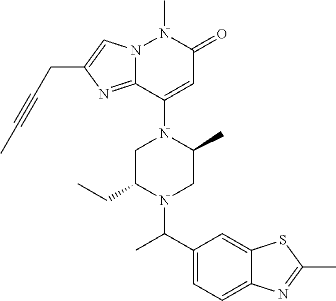 [SMILES: CC#CCc1cn2c(n1)c(N1C[C@@H](CC)N(C(C)c3ccc4nc(C)sc4c3)C[C@@H]1C)cc(=O)n2C] | 1,440 | 263 |
| A70a | 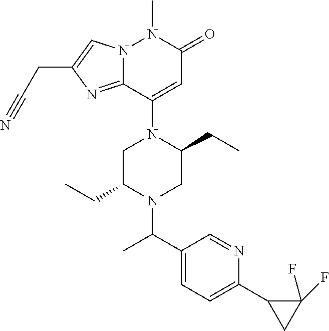 [SMILES: CC[C@H]1CN(C(C)c2ccc(C3CC3(F)F)nc2)[C@H](CC)CN1c1cc(=O)n(C)n2cc(CC#N)nc12] | 244 | 102 |
| A70b | 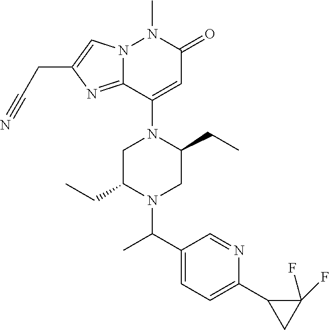 [SMILES: CC[C@H]1CN(C(C)c2ccc(C3CC3(F)F)nc2)[C@H](CC)CN1c1cc(=O)n(C)n2cc(CC#N)nc12] | 886 | 466 |
| A71 | 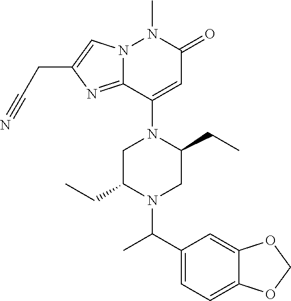 [SMILES: CC[C@H]1CN(C(C)c2ccc3c(c2)OCO3)[C@H](CC)CN1c1cc(=O)n(C)n2cc(CC#N)nc12] | 61 | 66 |
| A72 | 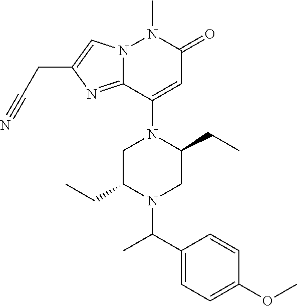 [SMILES: CC[C@H]1CN(C(C)c2ccc(OC)cc2)[C@H](CC)CN1c1cc(=O)n(C)n2cc(CC#N)nc12] | 76 | 23 |
| A73a | 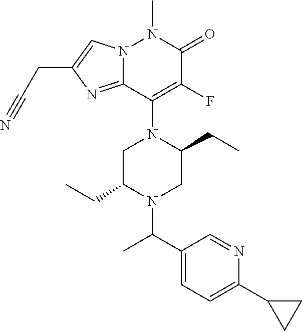 [SMILES: CC[C@H]1CN(C(C)c2ccc(C3CC3)nc2)[C@H](CC)CN1c1c(F)c(=O)n(C)n2cc(CC#N)nc12] | 77 | 177 |
| A73b | 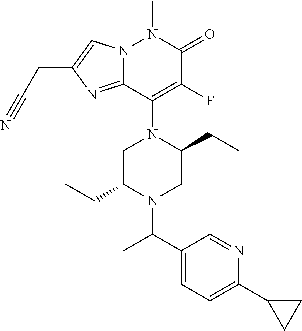 [SMILES: CC[C@H]1CN(C(C)c2ccc(C3CC3)nc2)[C@H](CC)CN1c1c(F)c(=O)n(C)n2cc(CC#N)nc12] | 302 | 832 |
| A74a | 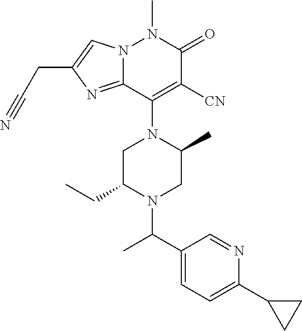 [SMILES: CC[C@@H]1CN(c2c(C#N)c(=O)n(C)n3cc(CC#N)nc23)[C@@H](C)CN1C(C)c1ccc(C2CC2)nc1] | 166 | 682 |
| A74b | 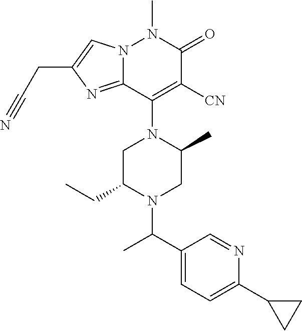 [SMILES: CC[C@@H]1CN(c2c(C#N)c(=O)n(C)n3cc(CC#N)nc23)[C@@H](C)CN1C(C)c1ccc(C2CC2)nc1] | 1,550 | 1,430 |
| A75a | 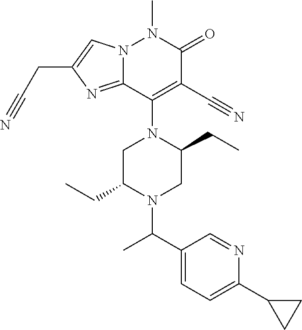 [SMILES: CC[C@H]1CN(C(C)c2ccc(C3CC3)nc2)[C@H](CC)CN1c1c(C#N)c(=O)n(C)n2cc(CC#N)nc12] | 276 | 420 |
| A75b | 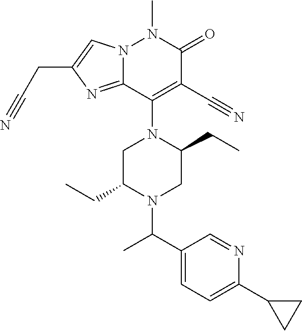 [SMILES: CC[C@H]1CN(C(C)c2ccc(C3CC3)nc2)[C@H](CC)CN1c1c(C#N)c(=O)n(C)n2cc(CC#N)nc12] | 1,050 | 1,300 |
| A76a | 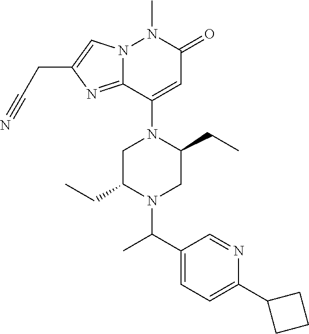 [SMILES: CC[C@H]1CN(C(C)c2ccc(C3CCC3)nc2)[C@H](CC)CN1c1cc(=O)n(C)n2cc(CC#N)nc12] | 53 | 111 |
| A76b | 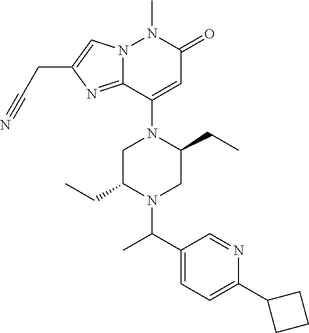 [SMILES: CC[C@H]1CN(C(C)c2ccc(C3CCC3)nc2)[C@H](CC)CN1c1cc(=O)n(C)n2cc(CC#N)nc12] | 780 | 842 |
| A77a | 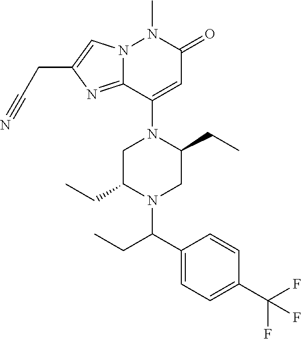 [SMILES: CCC(c1ccc(C(F)(F)F)cc1)N1C[C@H](CC)N(c2cc(=O)n(C)n3cc(CC#N)nc23)C[C@H]1CC] | 255 | 102 |
| A77b | 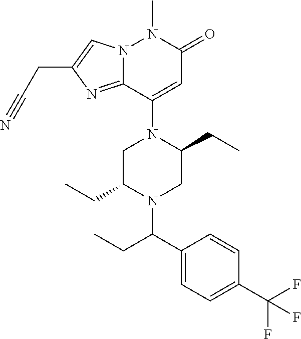 [SMILES: CCC(c1ccc(C(F)(F)F)cc1)N1C[C@H](CC)N(c2cc(=O)n(C)n3cc(CC#N)nc23)C[C@H]1CC] | 1,940 | 454 |
| A78a | 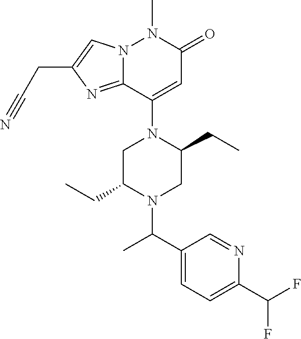 [SMILES: CC[C@H]1CN(C(C)c2ccc(C(F)F)nc2)[C@H](CC)CN1c1cc(=O)n(C)n2cc(CC#N)nc12] | 130 | 208 |
| A78b | 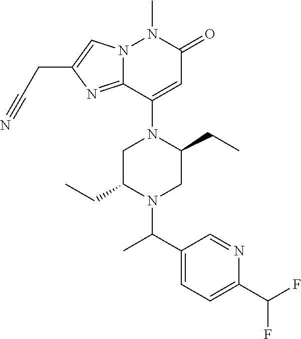 [SMILES: CC[C@H]1CN(C(C)c2ccc(C(F)F)nc2)[C@H](CC)CN1c1cc(=O)n(C)n2cc(CC#N)nc12] | 2,910 | 560 |
| A79a | 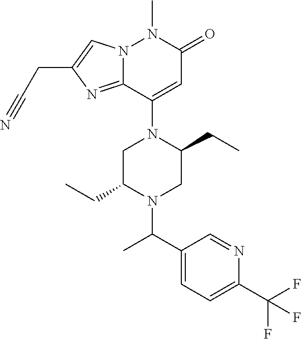 [SMILES: CC[C@H]1CN(C(C)c2ccc(C(F)(F)F)nc2)[C@H](CC)CN1c1cc(=O)n(C)n2cc(CC#N)nc12] | 112 | 355 |
| A79b | 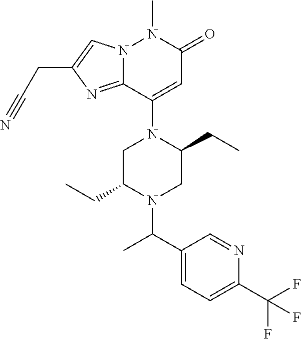 [SMILES: CC[C@H]1CN(C(C)c2ccc(C(F)(F)F)nc2)[C@H](CC)CN1c1cc(=O)n(C)n2cc(CC#N)nc12] | 1,600 | 521 |
| A80a | 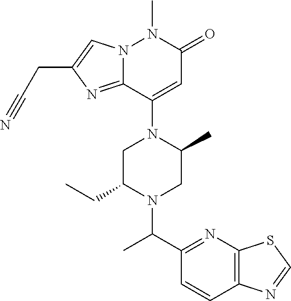 [SMILES: CC[C@@H]1CN(c2cc(=O)n(C)n3cc(CC#N)nc23)[C@@H](C)CN1C(C)c1ccc2ncsc2n1] | 44 | 31 |
| A80b | 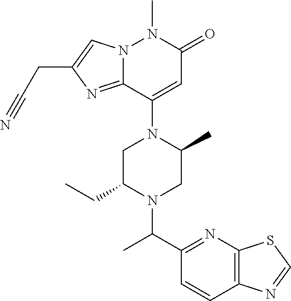 [SMILES: CC[C@@H]1CN(c2cc(=O)n(C)n3cc(CC#N)nc23)[C@@H](C)CN1C(C)c1ccc2ncsc2n1] | 8,160 | 1,730 |
| A81a | 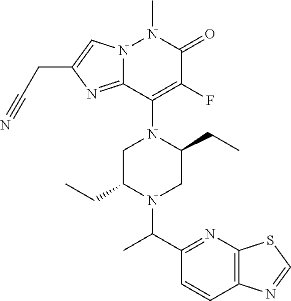 [SMILES: CC[C@H]1CN(C(C)c2ccc3ncsc3n2)[C@H](CC)CN1c1c(F)c(=O)n(C)n2cc(CC#N)nc12] | 1,260 | 56 |
| A81b | 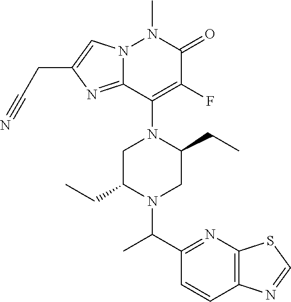 [SMILES: CC[C@H]1CN(C(C)c2ccc3ncsc3n2)[C@H](CC)CN1c1c(F)c(=O)n(C)n2cc(CC#N)nc12] | 32 | 18 |
| A82a | 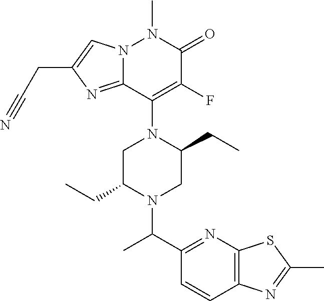 [SMILES: CC[C@H]1CN(C(C)c2ccc3nc(C)sc3n2)[C@H](CC)CN1c1c(F)c(=O)n(C)n2cc(CC#N)nc12] | 15 | 26 |
| A82b | 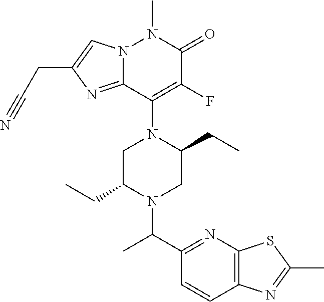 [SMILES: CC[C@H]1CN(C(C)c2ccc3nc(C)sc3n2)[C@H](CC)CN1c1c(F)c(=O)n(C)n2cc(CC#N)nc12] | 1,050 | 233 |
| A83 | 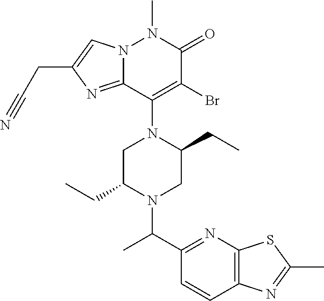 [SMILES: CC[C@H]1CN(C(C)c2ccc3nc(C)sc3n2)[C@H](CC)CN1c1c(Br)c(=O)n(C)n2cc(CC#N)nc12] | >10,000 | 42,600 |
| A84a | 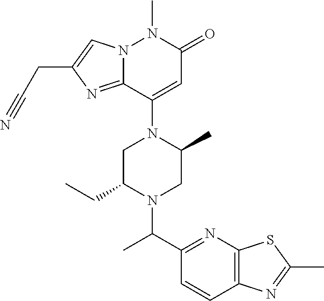 [SMILES: CC[C@@H]1CN(c2cc(=O)n(C)n3cc(CC#N)nc23)[C@@H](C)CN1C(C)c1ccc2nc(C)sc2n1] | 52 | 169 |
| A84b | 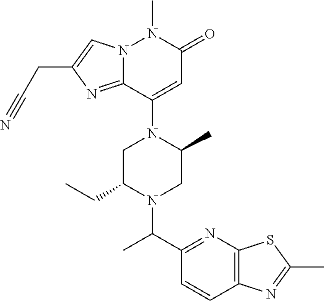 [SMILES: CC[C@@H]1CN(c2cc(=O)n(C)n3cc(CC#N)nc23)[C@@H](C)CN1C(C)c1ccc2nc(C)sc2n1] | 5,090 | 3,310 |
| A85 | 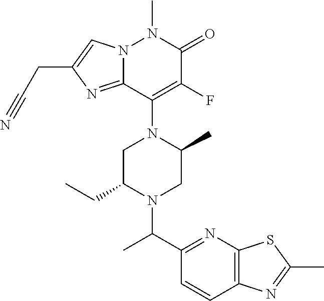 [SMILES: CC[C@@H]1CN(c2c(F)c(=O)n(C)n3cc(CC#N)nc23)[C@@H](C)CN1C(C)c1ccc2nc(C)sc2n1] | 119 | 444 |
| A86 | 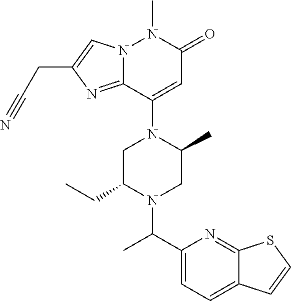 [SMILES: CC[C@@H]1CN(c2cc(=O)n(C)n3cc(CC#N)nc23)[C@@H](C)CN1C(C)c1ccc2ccsc2n1] | 36 | 18 |
| A87 | 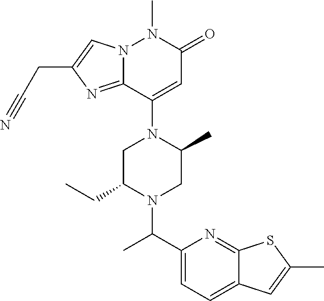 [SMILES: CC[C@@H]1CN(c2cc(=O)n(C)n3cc(CC#N)nc23)[C@@H](C)CN1C(C)c1ccc2cc(C)sc2n1] | 11 | 112 |
| A88 | 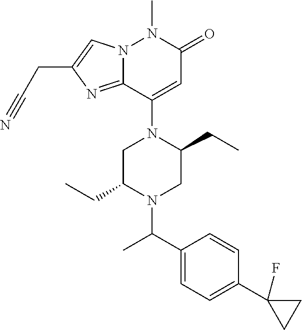 [SMILES: CC[C@H]1CN(C(C)c2ccc(C3(F)CC3)cc2)[C@H](CC)CN1c1cc(=O)n(C)n2cc(CC#N)nc12] | 39 | 72 |
| A89a | 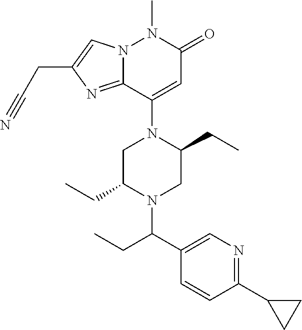 [SMILES: CCC(c1ccc(C2CC2)nc1)N1C[C@H](CC)N(c2cc(=O)n(C)n3cc(CC#N)nc23)C[C@H]1CC] | 73 | 171 |
| A89b | 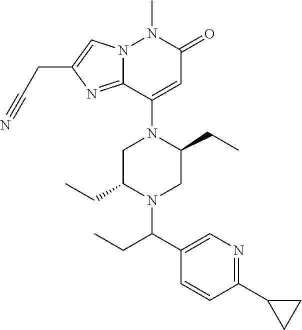 [SMILES: CCC(c1ccc(C2CC2)nc1)N1C[C@H](CC)N(c2cc(=O)n(C)n3cc(CC#N)nc23)C[C@H]1CC] | 1,010 | 1,520 |
| A90a | 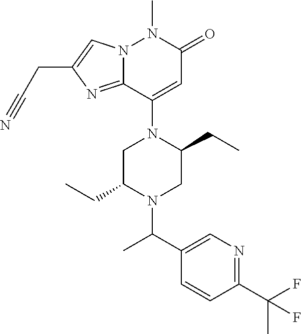 [SMILES: CC[C@H]1CN(C(C)c2ccc(C(C)(F)F)nc2)[C@H](CC)CN1c1cc(=O)n(C)n2cc(CC#N)nc12] | 25 | 121 |
| A90b | 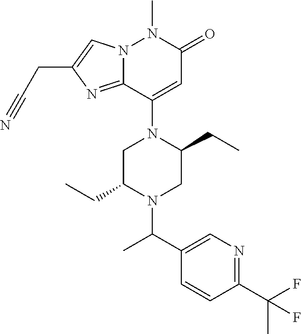 [SMILES: CC[C@H]1CN(C(C)c2ccc(C(C)(F)F)nc2)[C@H](CC)CN1c1cc(=O)n(C)n2cc(CC#N)nc12] | 802 | 386 |
| A91a | 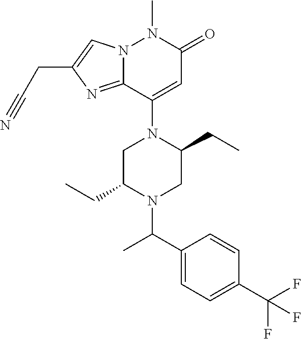 [SMILES: CC[C@H]1CN(C(C)c2ccc(C(F)(F)F)cc2)[C@H](CC)CN1c1cc(=O)n(C)n2cc(CC#N)nc12] | 73 | 155 |
| A91b | 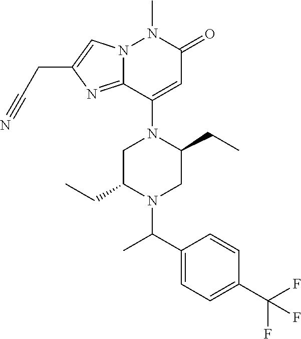 [SMILES: CC[C@H]1CN(C(C)c2ccc(C(F)(F)F)cc2)[C@H](CC)CN1c1cc(=O)n(C)n2cc(CC#N)nc12] | 1,430 | 319 |
| A92a | 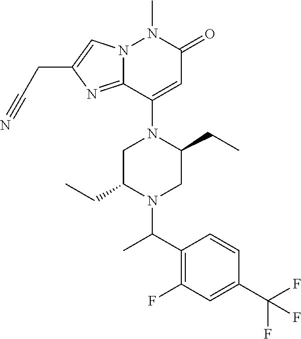 [SMILES: CC[C@H]1CN(C(C)c2ccc(C(F)(F)F)cc2F)[C@H](CC)CN1c1cc(=O)n(C)n2cc(CC#N)nc12] | 52 | 87 |
| A92b | 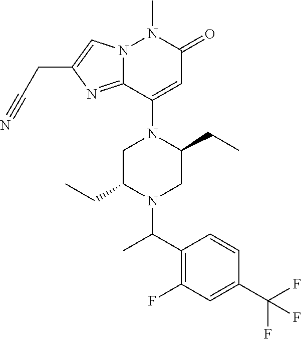 [SMILES: CC[C@H]1CN(C(C)c2ccc(C(F)(F)F)cc2F)[C@H](CC)CN1c1cc(=O)n(C)n2cc(CC#N)nc12] | 1,610 | 803 |
| A93a | 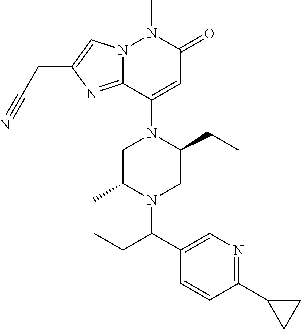 [SMILES: CCC(c1ccc(C2CC2)nc1)N1C[C@H](CC)N(c2cc(=O)n(C)n3cc(CC#N)nc23)C[C@H]1C] | 1,030 | 1,520 |
| A93b | 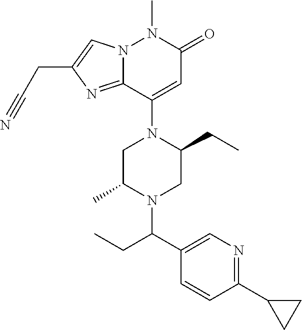 [SMILES: CCC(c1ccc(C2CC2)nc1)N1C[C@H](CC)N(c2cc(=O)n(C)n3cc(CC#N)nc23)C[C@H]1C] | 120 | 363 |
| A94a | 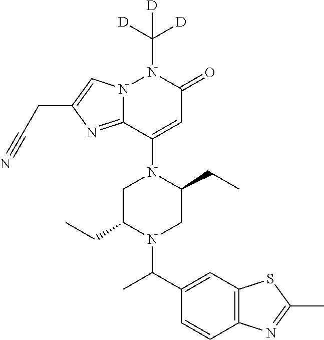 [SMILES: [2H]C([2H])([2H])n1c(=O)cc(N2C[C@@H](CC)N(C(C)c3ccc4nc(C)sc4c3)C[C@@H]2CC)c2nc(CC#N)cn21] | 24 | 108 |
| A94b | 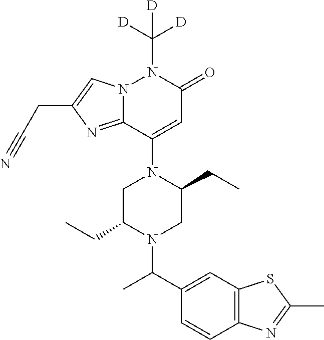 [SMILES: [2H]C([2H])([2H])n1c(=O)cc(N2C[C@@H](CC)N(C(C)c3ccc4nc(C)sc4c3)C[C@@H]2CC)c2nc(CC#N)cn21] | 1,150 | 3,790 |
| A95a | 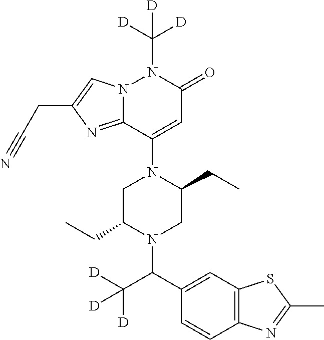 [SMILES: [2H]C([2H])([2H])C(c1ccc2nc(C)sc2c1)N1C[C@H](CC)N(c2cc(=O)n(C([2H])([2H])[2H])n3cc(CC#N)nc23)C[C@H]1CC] | 27 | 101 |
| A95b | 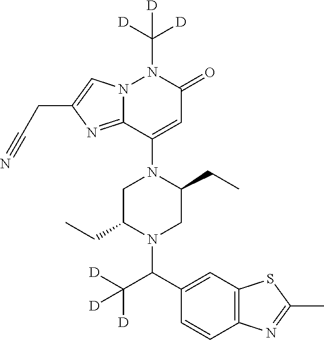 [SMILES: [2H]C([2H])([2H])C(c1ccc2nc(C)sc2c1)N1C[C@H](CC)N(c2cc(=O)n(C([2H])([2H])[2H])n3cc(CC#N)nc23)C[C@H]1CC] | 1,020 | 1,530 |
| A96a | 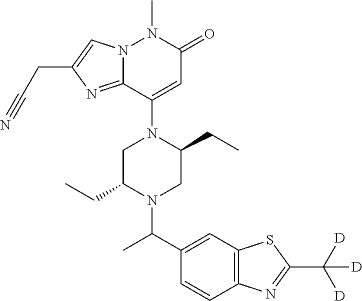 [SMILES: [2H]C([2H])([2H])c1nc2ccc(C(C)N3C[C@H](CC)N(c4cc(=O)n(C)n5cc(CC#N)nc45)C[C@H]3CC)cc2s1] | 33 | 54 |
| A96b | 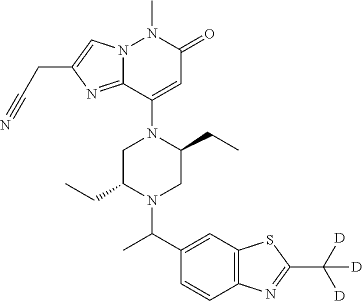 [SMILES: [2H]C([2H])([2H])c1nc2ccc(C(C)N3C[C@H](CC)N(c4cc(=O)n(C)n5cc(CC#N)nc45)C[C@H]3CC)cc2s1] | 861 | 2,080 |
| A97a | 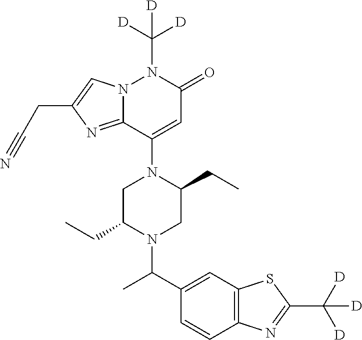 [SMILES: [2H]C([2H])([2H])c1nc2ccc(C(C)N3C[C@H](CC)N(c4cc(=O)n(C([2H])([2H])[2H])n5cc(CC#N)nc45)C[C@H]3CC)cc2s1] | 24 | 79 |
| A97b | 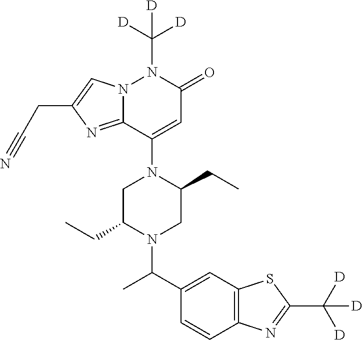 [SMILES: [2H]C([2H])([2H])c1nc2ccc(C(C)N3C[C@H](CC)N(c4cc(=O)n(C([2H])([2H])[2H])n5cc(CC#N)nc45)C[C@H]3CC)cc2s1] | 1,150 | 1,160 |
| A98a | 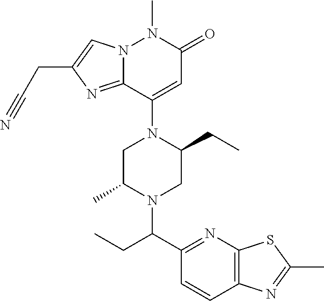 [SMILES: CCC(c1ccc2nc(C)sc2n1)N1C[C@H](CC)N(c2cc(=O)n(C)n3cc(CC#N)nc23)C[C@H]1C] | 2,940 | 712 |
| A98b | 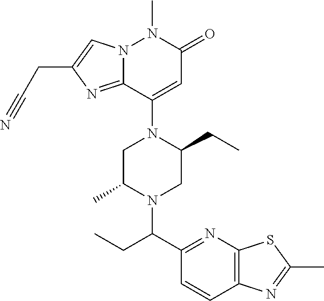 [SMILES: CCC(c1ccc2nc(C)sc2n1)N1C[C@H](CC)N(c2cc(=O)n(C)n3cc(CC#N)nc23)C[C@H]1C] | 45 | 39 |
| A99a | 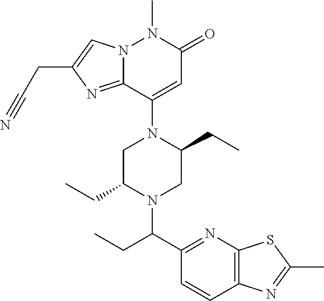 [SMILES: CCC(c1ccc2nc(C)sc2n1)N1C[C@H](CC)N(c2cc(=O)n(C)n3cc(CC#N)nc23)C[C@H]1CC] | 132 | 196 |
| A99b | 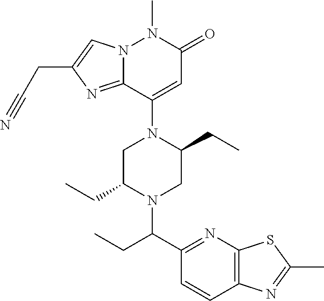 [SMILES: CCC(c1ccc2nc(C)sc2n1)N1C[C@H](CC)N(c2cc(=O)n(C)n3cc(CC#N)nc23)C[C@H]1CC] | 11 | 21 |
| A100a | 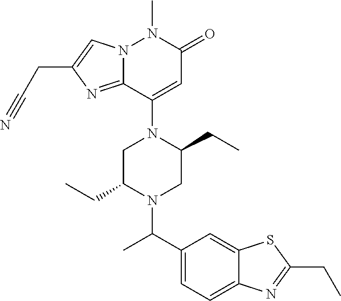 [SMILES: CCc1nc2ccc(C(C)N3C[C@H](CC)N(c4cc(=O)n(C)n5cc(CC#N)nc45)C[C@H]3CC)cc2s1] | 19 | 297 |
| A100b | 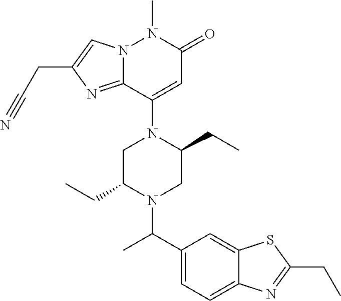 [SMILES: CCc1nc2ccc(C(C)N3C[C@H](CC)N(c4cc(=O)n(C)n5cc(CC#N)nc45)C[C@H]3CC)cc2s1] | 911 | 1,060 |
| A101a | 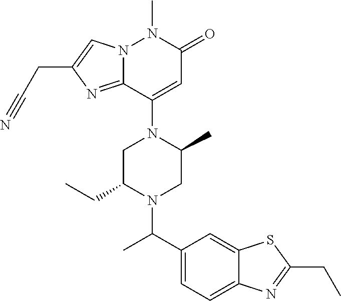 [SMILES: CCc1nc2ccc(C(C)N3C[C@H](C)N(c4cc(=O)n(C)n5cc(CC#N)nc45)C[C@H]3CC)cc2s1] | 29 | 851 |
| A101b | 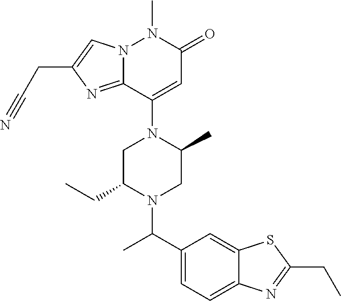 [SMILES: CCc1nc2ccc(C(C)N3C[C@H](C)N(c4cc(=O)n(C)n5cc(CC#N)nc45)C[C@H]3CC)cc2s1] | 1,110 | 2,360 |
| A102a | 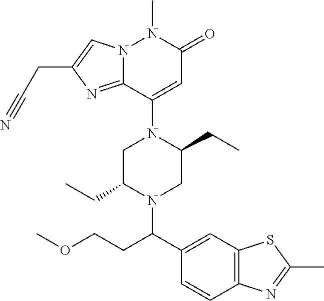 [SMILES: CC[C@H]1CN(C(CCOC)c2ccc3nc(C)sc3c2)[C@H](CC)CN1c1cc(=O)n(C)n2cc(CC#N)nc12] | 46 | 378 |
| A102b | 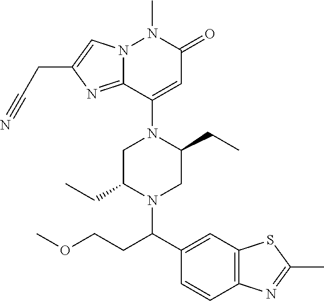 [SMILES: CC[C@H]1CN(C(CCOC)c2ccc3nc(C)sc3c2)[C@H](CC)CN1c1cc(=O)n(C)n2cc(CC#N)nc12] | 623 | 735 |
| A103 | 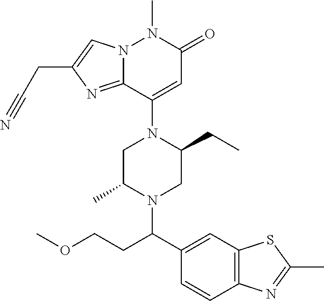 [SMILES: CC[C@H]1CN(C(CCOC)c2ccc3nc(C)sc3c2)[C@H](C)CN1c1cc(=O)n(C)n2cc(CC#N)nc12] | 138 | 701 |
| A104 | 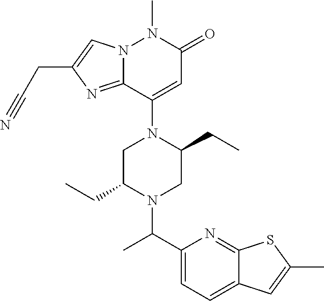 [SMILES: CC[C@H]1CN(C(C)c2ccc3cc(C)sc3n2)[C@H](CC)CN1c1cc(=O)n(C)n2cc(CC#N)nc12] | 11 | 26 |
| A105a | 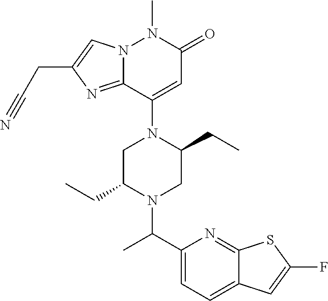 [SMILES: CC[C@H]1CN(C(C)c2ccc3cc(F)sc3n2)[C@H](CC)CN1c1cc(=O)n(C)n2cc(CC#N)nc12] | 706 | 195 |
| A105b | 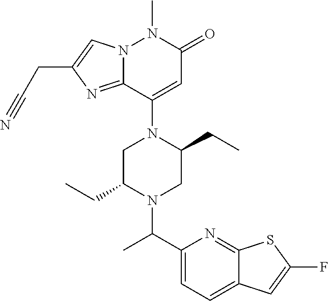 [SMILES: CC[C@H]1CN(C(C)c2ccc3cc(F)sc3n2)[C@H](CC)CN1c1cc(=O)n(C)n2cc(CC#N)nc12] | 48 | 3.3 |
| A106 | 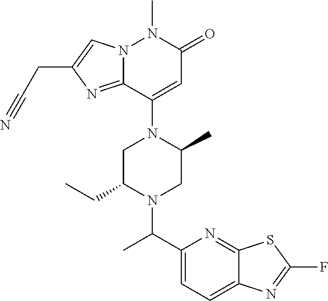 [SMILES: CC[C@@H]1CN(c2cc(=O)n(C)n3cc(CC#N)nc23)[C@@H](C)CN1C(C)c1ccc2nc(F)sc2n1] | 96 | 26 |
| A107a | 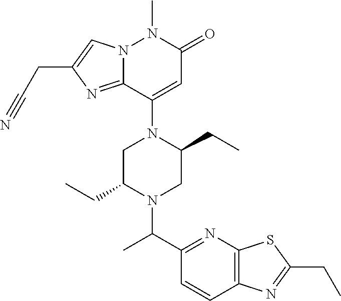 [SMILES: CCc1nc2ccc(C(C)N3C[C@H](CC)N(c4cc(=O)n(C)n5cc(CC#N)nc45)C[C@H]3CC)nc2s1] | 1,130 | 1,130 |
| A107b | 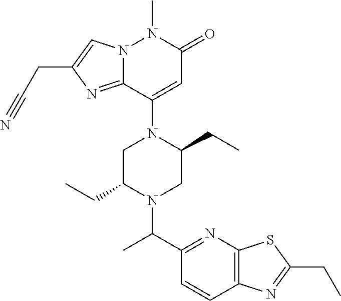 [SMILES: CCc1nc2ccc(C(C)N3C[C@H](CC)N(c4cc(=O)n(C)n5cc(CC#N)nc45)C[C@H]3CC)nc2s1] | 11 | 20 |
| A108a | 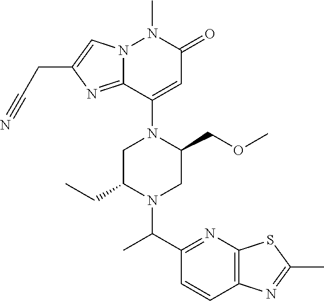 [SMILES: CC[C@@H]1CN(c2cc(=O)n(C)n3cc(CC#N)nc23)[C@@H](COC)CN1C(C)c1ccc2nc(C)sc2n1] | >10,000 | 6,400 |
| A108b | 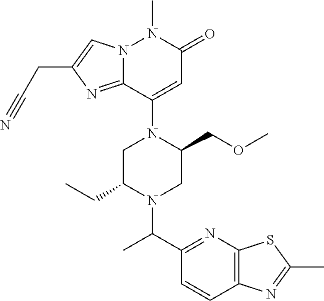 [SMILES: CC[C@@H]1CN(c2cc(=O)n(C)n3cc(CC#N)nc23)[C@@H](COC)CN1C(C)c1ccc2nc(C)sc2n1] | 182 | 510 |
| A109a | 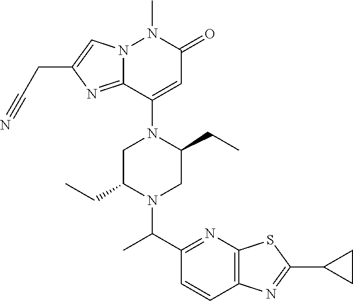 [SMILES: CC[C@H]1CN(C(C)c2ccc3nc(C4CC4)sc3n2)[C@H](CC)CN1c1cc(=O)n(C)n2cc(CC#N)nc12] | 1,800 | 1,490 |
| A109b | 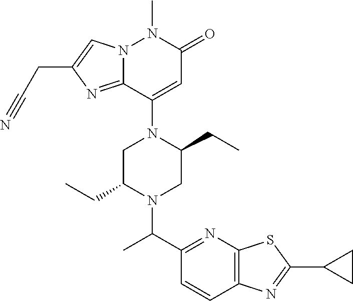 [SMILES: CC[C@H]1CN(C(C)c2ccc3nc(C4CC4)sc3n2)[C@H](CC)CN1c1cc(=O)n(C)n2cc(CC#N)nc12] | 28 | 113 |
| A110a | 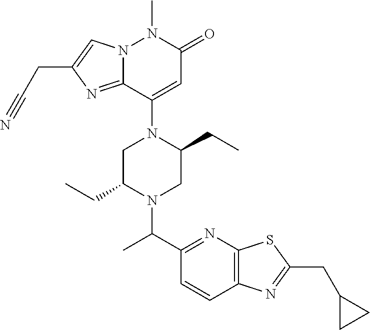 [SMILES: CC[C@H]1CN(C(C)c2ccc3nc(CC4CC4)sc3n2)[C@H](CC)CN1c1cc(=O)n(C)n2cc(CC#N)nc12] | 1,170 | 1,070 |
| A110b | 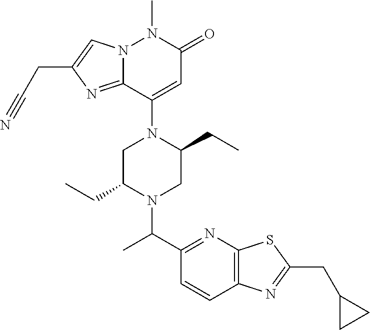 [SMILES: CC[C@H]1CN(C(C)c2ccc3nc(CC4CC4)sc3n2)[C@H](CC)CN1c1cc(=O)n(C)n2cc(CC#N)nc12] | 15 | 61 |
| A111 | 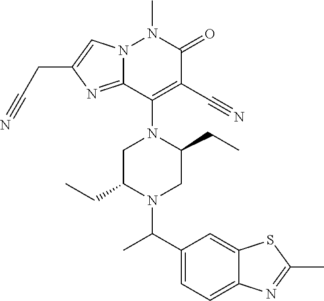 [SMILES: CC[C@H]1CN(C(C)c2ccc3nc(C)sc3c2)[C@H](CC)CN1c1c(C#N)c(=O)n(C)n2cc(CC#N)nc12] | 132 | 575 |
| A112a | 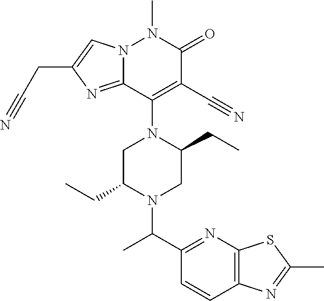 [SMILES: CC[C@H]1CN(C(C)c2ccc3nc(C)sc3n2)[C@H](CC)CN1c1c(C#N)c(=O)n(C)n2cc(CC#N)nc12] | 3,500 | 752 |
| A112b | 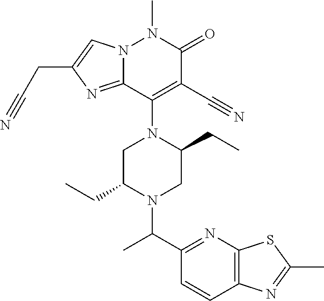 [SMILES: CC[C@H]1CN(C(C)c2ccc3nc(C)sc3n2)[C@H](CC)CN1c1c(C#N)c(=O)n(C)n2cc(CC#N)nc12] | 118 | 60 |
| A113a | 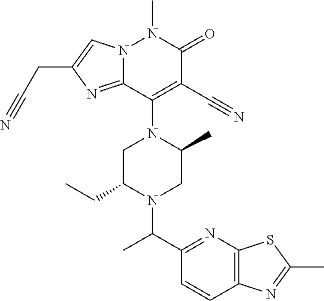 [SMILES: CC[C@@H]1CN(c2c(C#N)c(=O)n(C)n3cc(CC#N)nc23)[C@@H](C)CN1C(C)c1ccc2nc(C)sc2n1] | 118 | 185 |
| A113b | 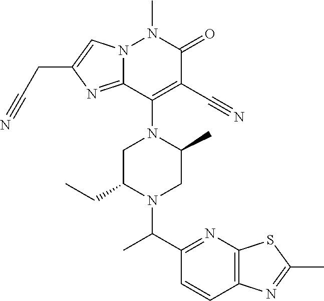 [SMILES: CC[C@@H]1CN(c2c(C#N)c(=O)n(C)n3cc(CC#N)nc23)[C@@H](C)CN1C(C)c1ccc2nc(C)sc2n1] | 2,910 | 651 |
| A114 | 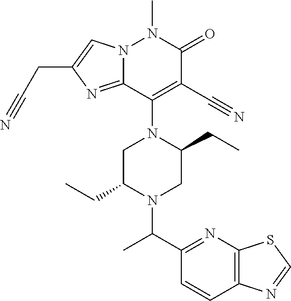 [SMILES: CC[C@H]1CN(C(C)c2ccc3ncsc3n2)[C@H](CC)CN1c1c(C#N)c(=O)n(C)n2cc(CC#N)nc12] | 127 | 68 |
| A115a | 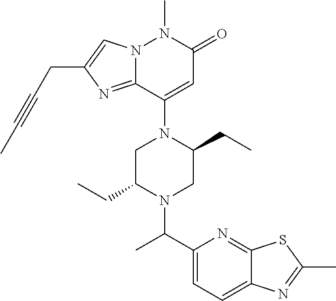 [SMILES: CC#CCc1cn2c(n1)c(N1C[C@@H](CC)N(C(C)c3ccc4nc(C)sc4n3)C[C@@H]1CC)cc(=O)n2C] | >10,000 | 288 |
| A115b | 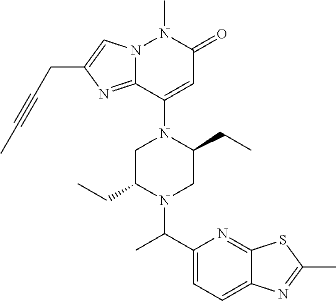 [SMILES: CC#CCc1cn2c(n1)c(N1C[C@@H](CC)N(C(C)c3ccc4nc(C)sc4n3)C[C@@H]1CC)cc(=O)n2C] | 49 | 2.7 |
| A116a | 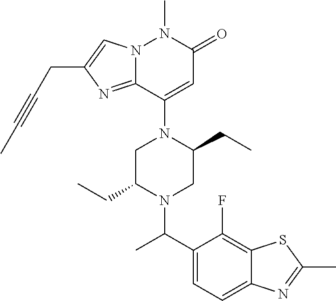 [SMILES: CC#CCc1cn2c(n1)c(N1C[C@@H](CC)N(C(C)c3ccc4nc(C)sc4c3F)C[C@@H]1CC)cc(=O)n2C] | 132 | 118 |
| A116b | 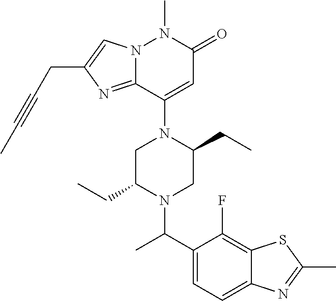 [SMILES: CC#CCc1cn2c(n1)c(N1C[C@@H](CC)N(C(C)c3ccc4nc(C)sc4c3F)C[C@@H]1CC)cc(=O)n2C] | >10,000 | 708 |
| A117a | 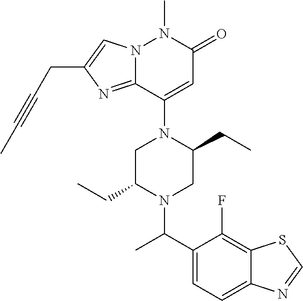 [SMILES: CC#CCc1cn2c(n1)c(N1C[C@@H](CC)N(C(C)c3ccc4ncsc4c3F)C[C@@H]1CC)cc(=O)n2C] | 48 | 22 |
| A117b | 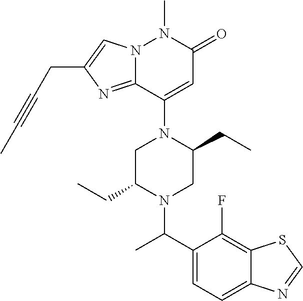 [SMILES: CC#CCc1cn2c(n1)c(N1C[C@@H](CC)N(C(C)c3ccc4ncsc4c3F)C[C@@H]1CC)cc(=O)n2C] | 2,000 | 535 |
| A118 | 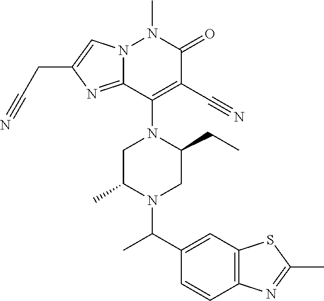 [SMILES: CC[C@H]1CN(C(C)c2ccc3nc(C)sc3c2)[C@H](C)CN1c1c(C#N)c(=O)n(C)n2cc(CC#N)nc12] | 105 | 407 |
| A119a | 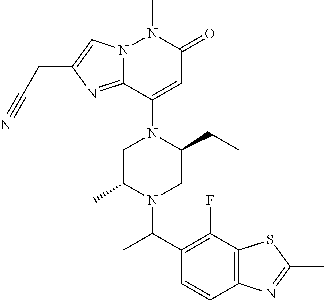 [SMILES: CC[C@H]1CN(C(C)c2ccc3nc(C)sc3c2F)[C@H](C)CN1c1cc(=O)n(C)n2cc(CC#N)nc12] | 1,460 | 2,050 |
| A119b | 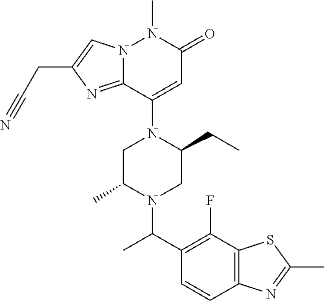 [SMILES: CC[C@H]1CN(C(C)c2ccc3nc(C)sc3c2F)[C@H](C)CN1c1cc(=O)n(C)n2cc(CC#N)nc12] | 51 | 340 |
| A120a | 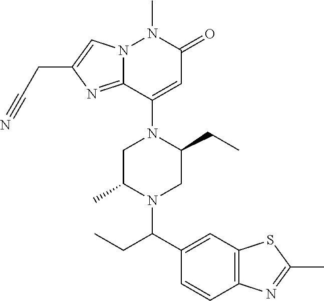 [SMILES: CCC(c1ccc2nc(C)sc2c1)N1C[C@H](CC)N(c2cc(=O)n(C)n3cc(CC#N)nc23)C[C@H]1C] | 1,310 | 2,290 |
| A120b | 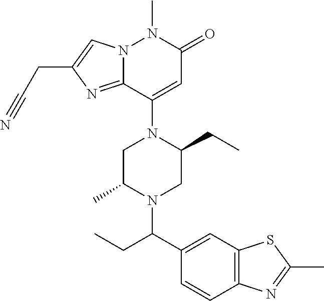 [SMILES: CCC(c1ccc2nc(C)sc2c1)N1C[C@H](CC)N(c2cc(=O)n(C)n3cc(CC#N)nc23)C[C@H]1C] | 182 | 138 |
| A121a | 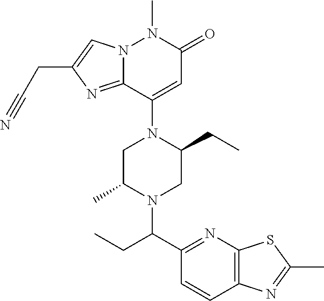 [SMILES: CCC(c1ccc2nc(C)sc2n1)N1C[C@H](CC)N(c2cc(=O)n(C)n3cc(CC#N)nc23)C[C@H]1C] | 7,160 | 3,170 |
| A121b | 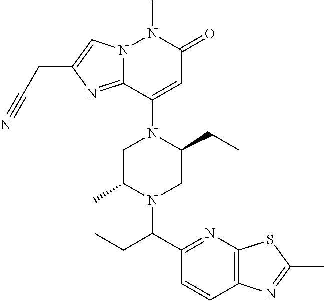 [SMILES: CCC(c1ccc2nc(C)sc2n1)N1C[C@H](CC)N(c2cc(=O)n(C)n3cc(CC#N)nc23)C[C@H]1C] | 143 | 50 |
| A122a | 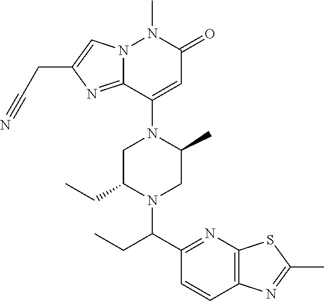 [SMILES: CCC(c1ccc2nc(C)sc2n1)N1C[C@H](C)N(c2cc(=O)n(C)n3cc(CC#N)nc23)C[C@H]1CC] | 38 | 110 |
| A122b | 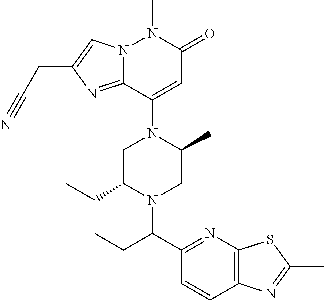 [SMILES: CCC(c1ccc2nc(C)sc2n1)N1C[C@H](C)N(c2cc(=O)n(C)n3cc(CC#N)nc23)C[C@H]1CC] | 2,350 | 1,170 |
| A123a | 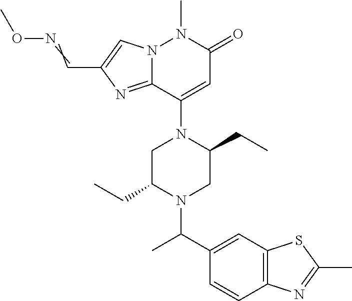 | 4,890 | 269 |
| A123bc | 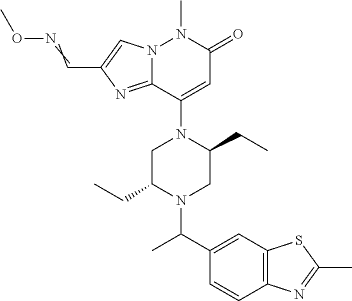 | 2,390 | 131 |
| A123d | 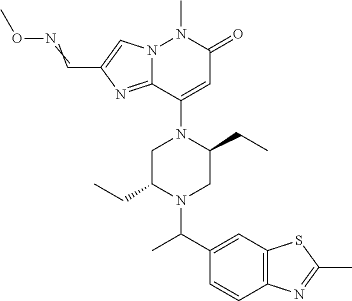 | >10,000 | 749 |
| A124a | 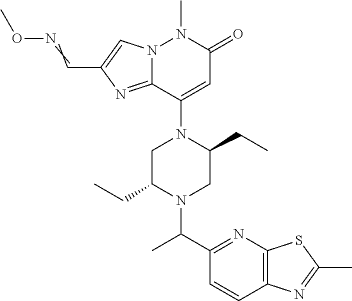 | >10,000 | 2,000 |
| A124bc | 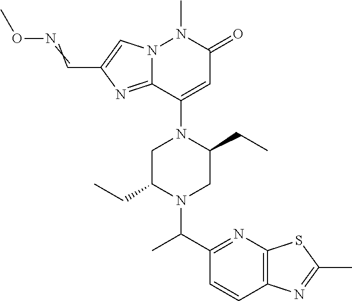 | 8,100 | 78 |
| A124d | 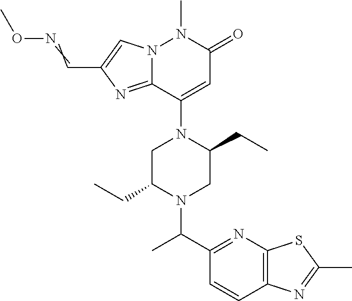 | 630 | 5.9 |

### Cell Culture and DGKα/(Knockout Jurkat Cell Line Construction

Jurkat cells and human PBMC were maintained in RPMI 1640 medium (Gibco) supplemented with 10% fetal bovine serum (FBS, Thermo Scientific), 100 units/mL penicillin and 0.1 mg/mL streptomycin (Gibco) in a humidified 37° C. environment with 5% CO₂. HepG2-OS8 cells, which express the single chain variable fragment (scFv) of an anti-human CD3 mAb OKT3 fused to the C-terminal domain (113-220) of mouse CD8a ωhich includes hinge, transmembrane and cytoplasmic domains, were maintained in MEM medium (Gibco) supplemented with 10% fetal bovine serum (FBS, Thermo Scientific), 100 units/mL penicillin and 0.1 mg/mL streptomycin (Gibco) in a humidified 37° C. environment with 5% CO₂.

Jurkat cells were infected with the lentivirus expressing spCas9 and sgRNA targeting human DGKα or DGKζ. Cell clones that stably knockout with DGKα/ζ were established and maintained in the RPMI 1640 complete medium. Knockout efficiency of eSPCas9-Lenticrispr DGKα or DGKζ sgRNA in single cell clone was determined using genomic sequencing and immunoblotting method. Selected compounds did not induce DGKα or DGKζ independent IL-2 production in DGKα/ζ KO jurkat cells.

### Non-Stimulated Phosphorylated ERK Detection Assay

Cellular non-stimulated phospho-ERK were measured using a AlphaLISA-based method (Beaudet, Lucille, et al. Nature Methods. 2008, 5.12: an8-an9). Jurkat cells were subcultured in T75 flasks. The next day, growth medium was replaced to serum free RPMI 1640 for 4 hours or overnight. The cells were then seeded into 96-well plates and treated with compounds. After 2 h compound treatment, lysis buffer (PerkinElmer) was added to each well. Plates were then incubated at room temperature with shaking for 30 minutes. A total of 10 μL of cell lysate from each well of a 96-well plate was transferred to a 384-well white assay plate. Phosphor-ERK was quantitated using the AlphaLISA kit (Cat #ALSU-PERK-A10K) as described by the manufacturer manual (PerkinElmer). AlphaLISA signals were measured using a PHERAstar FSX reader (BMG Labtech). Selected compounds did not elevate ERK phosphorylation in Jurkat cells without TCR activation.

### IL-2 Production Assay in Human PBMC

Frozen human PBMC were thawed in RPMI 1640 medium and incubated at 37° C. overnight. OS8 overexpressing HepG2 cells were seeded into 384-well plates overnight. The next day, PBMC were added into the 384-well plates and then treated with compounds. PBMC and HepG2-OS8 cells were co-cultured for 48 hours at 37° C. Culture supernatant was collected for subsequent measurement of IL-2 concentration by a TR-FRET-based method (Degorce, François, et al. Current chemical genomics. 2009, 3: 22) as described by the manufacturer manual (Cisbio). FRET signals were measured using a PHERAstar FSX reader (BMG Labtech). Selected compounds showed good potency in Human PBMC assay.

### Explorative Acute Toxicity Study in BALB/c Mice

The test compound was dissolved in vehicle formulation (DMA: 30% solutol HS-15(w/v):saline=20:20:60) and injected through tail vein into BALB/c mice at the doses of 2 and/or 10 mg/kg. Continuous clinical observation within 2 hours post injection was performed. Selected compounds were well tolerant at the doses of 2 and/or 10 mg/kg.

It is to be understood that, if any prior art publication is referred to herein, such reference does not constitute an admission that the publication forms a part of the common general knowledge in the art in any country.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

The disclosures of all publications, patents, patent applications and published patent applications referred to herein by an identifying citation are hereby incorporated herein by reference in their entirety.

What is claimed is:

1. A compound of formula (I), 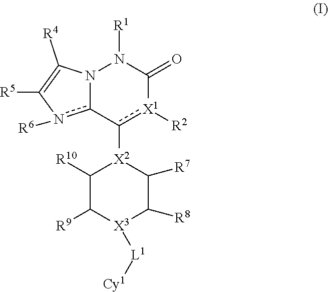
or a stereoisomer or pharmaceutically acceptable salt thereof
wherein
X¹ is C or N,
each of X² and X³ is independently selected form —N— or —CH—;
the symbol [symbol] is a single or double bond,
R¹ is hydrogen, deuterium, halogen, substituted or unsubstituted alkyl, substituted or unsubstituted alkoxyl, substituted or unsubstituted cycloalkyl, substituted or unsubstituted aryl, substituted or unsubstituted heterocyclyl, or substituted or unsubstituted heteroaryl;
R² is hydrogen, halogen, cyano, substituted or unsubstituted alkyl, substituted or unsubstituted alkoxyl, substituted or unsubstituted cycloalkyl, substituted or unsubstituted aryl, substituted or unsubstituted heterocyclyl, or substituted or unsubstituted heteroaryl, provided that R² is absent when X¹ is N and the bond [symbol] attached to X¹ is a double bond;
R⁴ is hydrogen, halogen, cyano, substituted or unsubstituted alkyl, substituted or unsubstituted alkoxyl, substituted or unsubstituted cycloalkyl, substituted or unsubstituted aryl, substituted or unsubstituted heterocyclyl, or substituted or unsubstituted heteroaryl;
R⁵ is hydrogen, halogen, cyano, substituted or unsubstituted alkyl, substituted or unsubstituted alkoxyl, substituted or unsubstituted alkenyl, substituted or unsubstituted alkynyl, substituted or unsubstituted cycloalkyl, substituted or unsubstituted aryl, substituted or unsubstituted heterocyclyl, or substituted or unsubstituted heteroaryl, R^{5a}—C(O)—, R^{5a}—C(O)O—, R^{5a}—O—C(O)—, R^{5a}—C(O)NR^{5b}—, R^{5a}—NR^{5b}—C(O)—, R^{5a}—SO₂— or, R^{5a}—O—N═CH—, wherein R^{5a} and R^{5b} are each independently hydrogen, alkyl, or cycloalkyl;
R⁶ is absent, hydrogen, halogen, or alkyl which is unsubstituted or substituted with halogen or cyano, provided that R⁶ is absent when the bond [symbol] attached to the nitrogen to which R⁶ is attached is a double bond;
each of R⁷, R⁹, R⁸, and R¹⁰ is independently hydrogen, cyano, substituted or unsubstituted alkyl, substituted or unsubstituted alkoxyl, substituted or unsubstituted cycloalkyl, —C(O)R^{7a}, or -alkyl-C(O)R^{7a}, and wherein R^{7a} is hydrogen, alkyl, or alkoxy, provided that at least one of R⁷ and R⁹ is not hydrogen;
or R⁷ and R⁹ are each hydrogen and R⁸ and R¹⁰ together form a bridge containing at least one —CH₂-moiety in addition to the two bridgehead atoms; or R⁸ and R¹⁰ are each hydrogen and R⁷ and R⁹ together form a bridge containing at least one —CH₂— moiety in addition to the two bridgehead atoms;
L¹ is a direct bond, —O—, —N(R^{L})—, substituted or unsubstituted alkyl, -alkylene or —C(O)—, wherein R^{L} is hydrogen or alkyl;
Cy¹ is unsubstituted or substituted cycloalkyl, unsubstituted or substituted aryl, unsubstituted or substituted heterocyclyl, or unsubstituted or substituted heteroaryl, wherein Cy¹ is optionally substituted with one, two or three substituents R^{3a},
wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, cycloalkyl or heterocyclyl;
optionally wherein two R^{3a} connect to the same carbon and together form a spirocyclic ring;
optionally wherein two R^{3a} form a fused ring with Cy¹, wherein R^{3a} is unsubstituted or substituted with cyano, alkoxy, alkyl, halogen, or hydroxy; and
wherein R^{3b} and R^{3c} are each independently hydrogen or alkyl.

2. The compound of claim 1, wherein the compound of formula (I) is a compound of formula (II):

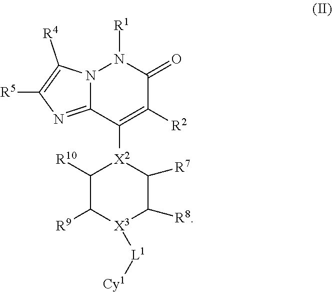

3. The compound of claim 2, wherein the compound of formula (II) is a compound of formula (III):

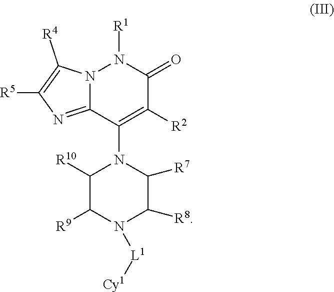

4. The compound of claim 1, wherein the compound of formula (I) is a compound of formula (IV):

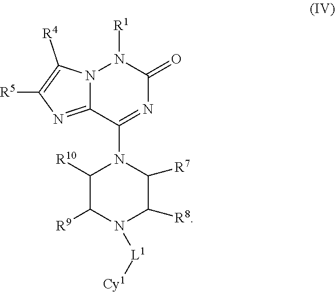

5. The compound of claim 1, wherein the compound of formula (I) is a compound of formula (V):

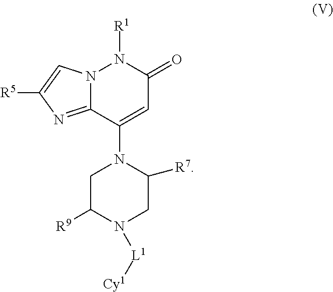

6. The compound of claim 1, wherein the compound of formula (I) is a compound of formula (VI):

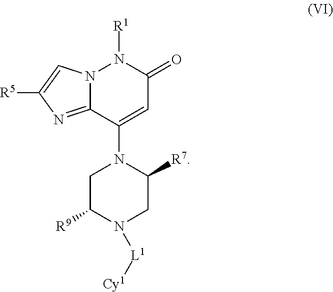

7. The compound of any one of claims 1-6, wherein R¹ is hydrogen, or substituted or unsubstituted alkyl.

8. The compound of any one of claims 1-6, wherein R¹ is hydrogen, or C₁₋₄alkyl optionally substituted with deuterium, halogen, hydroxy, alkoxy or cycloalkyl; preferably R¹ is hydrogen, or C₁₋₃alkyl optionally substituted with deuterium, or halogen; more preferably R¹ is hydrogen, or C₁₋₃ alkyl optionally substituted with deuterium.

9. The compound of any one of claims 1-8, wherein R¹ is hydrogen, methyl, methyl-d3, ethyl, isopropyl, 2-hydroxyethyl, 2-methoxyethyl, 2,2,2-trifluoroethyl, 2,2-difluoroethyl, or cyclopropylmethyl; preferably R¹ is hydrogen, methyl, ethyl or methyl-d3; more preferably R¹ is methyl.

10. The compound of any one of claims 1-9, wherein R² is hydrogen, halogen, C₁₋₄alkyl, C₁₋₄ alkoxyl or cyano; preferably R² is hydrogen, F, Br, Cl or CN; more preferably R² is hydrogen, F, or CN; even more preferably R² is hydrogen.

11. The compound of any one of claims 1-10, wherein R⁴ is hydrogen, halogen or alkyl, wherein the alkyl is optionally substituted with halogen or —OR^{4a}, wherein R^{4a} is hydrogen, alkyl, alkenyl, alkynyl, cycloalkyl or heterocycloalkyl, wherein alkyl, alkenyl, alkynyl, cycloalkyl or heterocycloalkyl is optionally substituted with —C₁₋₆aklyl, —C₁₋₆alkoxy or —C₃₋₈cycloalkyl; preferably R⁴ is hydrogen, halogen or C₁₋₄alkyl, wherein the alkyl is optionally substituted with halogen or —OR^{4a}; more preferably R⁴ is hydrogen, halogen or C₁₋₄alkyl, wherein the alkyl is optionally substituted with halogen; more preferably R⁴ is hydrogen.

12. The compound of any one of claims 1-11, wherein R⁴ is hydrogen, fluoro, chloro, bromo, methyl, trifluoromethyl, ethyl, or 2,2,2-trifluoroethyl; preferably R⁴ is hydrogen.

13. The compound of any one of claims 1-12, wherein R⁵ is hydrogen, alkyl, alkenyl, alkynyl, cycloalkyl, R^{5a}—C(O)—, R^{5a}—C(O)O—, R^{5a}—O—C(O)—, R^{5a}—C(O)NR^{5b}—, R^{5a}—NR^{5b}—C(O)—, R^{5a}—SO₂—, R^{5a}—O—N═CH—or heterocyclyl,
wherein said alkyl is unsubstituted or substituted with cyano, —C(O)OR^{5c}, —C(O)R^{5c}, —S(═O)—R^{5c}, —S(═O)₂—R^{5c}, —S(═O)(═NH)—R^{5c}, —C(O)NR^{5c}R^{5d}, heterocyclyl, alkoxy, hydroxy, cycloalkyl, or NR^{5c}R^{5d}; and
wherein each of said cycloalkyl and heterocyclyl is unsubstituted or substituted with alkyl, cyano or halogen substituted alkyl, cyano, —C(O)OR^{5c}, —C(O)R^{5c}, —C(O)NR^{5c}R^{5d}, heterocyclyl, alkoxy, hydroxy, cycloalkyl, NR^{5c}R^{5d}, or R^{5c}—SO₂—,
wherein R^{5a} and R^{5b} are each independently hydrogen, alkyl, or cycloalkyl; and wherein R^{5c} and R^{5d} are hydrogen or alkyl.

14. The compound of any one of claims 1-13, wherein R⁵ is hydrogen, alkyl, alkenyl or alkynyl, wherein said alkyl is unsubstituted or substituted with cyano; preferably R⁵ is C₁₋₄alkyl, C₂₋₄alkenyl or C₂₋₄alkynyl, wherein said alkyl is substituted with cyano.

15. The compound of any one of claims 1-14, wherein R⁵ is hydrogen, —CH₂—CN, —CH₂C(O)—OMe, —CH(CH₃)CN, oxiran-2-ylmethyl-, prop-2-yn-1-yl, but-2-yn-1-yl, prop-1-en-2-yl, methyl, isopropyl, —CH₂CH₂—O—CH₃, —CH₂—O—CH₃, —CH(CH₃)—O—CH₃, —CH₂C(O)NH₂, —CH₂CH₂—OH, —CH₂—OH, cyclopropyl-CH₂—, —CH₂CH₂N(CH₃)₂, CH₃—SO₂—, cyclopropyl, cyclobutyl, cyclopropyl-C(O)—, 1-cyanocyclopropyl, 2-cyanocyclopropyl, 2-cyanocyclobutyl, 3-(cyanomethyl)-1-(ethylsulfonyl)azetidine-3-yl, 1-cyano-2-cyclopentyleth-2-yl, —CH₂—N(CH₃)₂, —CH═NO—CH₃, —CH₂—S(═O)—CH₃, —CH₂—S(═O)₂—CH₃, or —CH₂—S(═O)(═NH)—CH₃; preferably, R⁵ is hydrogen, CN—CH₂—, —CH₂C(O)—OMe, —CH(CH₃)CN, oxiran-2-ylmethyl-, prop-2-yn-1-yl, but-2-yn-1-yl, prop-1-en-2-yl, —CH₂—OH, —CH(CH₃)CN, —CH₂—O—CH₃, —CH(CH₃)—O—CH₃, —CH₂—N(CH₃)₂, —CH═NO—CH₃, —CH₂—S(═O)—CH₃, —CH₂—S(═O)₂—CH₃, or —CH₂—S(═O)(═NH)—CH₃; more preferably, R⁵ is CN—CH₂—, prop-2-yn-1-yl, but-2-yn-1-yl, or prop-1-en-2-yl, —CH₂—OH, —CH(CH₃)CN, —CH₂—O—CH₃, —CH(CH₃)—O—CH₃, —CH₂—N(CH₃)₂, —CH═NO—CH₃, —CH₂—S(═O)—CH₃, —CH₂—S(═O)₂—CH₃, or —CH₂—S(═O)(═NH)—CH₃; more preferably R⁵ is CN—CH₂—, —CH₂—OH, —CH(CH₃)CN, —CH₂—O—CH₃, —CH(CH₃)—O—CH₃, —CH₂—N(CH₃)₂, —CH═NO—CH₃, —CH₂—S(═O)—CH₃, —CH₂—S(═O)₂—CH₃, or —CH₂—S(═O)(═NH)—CH₃; more preferably R⁵ is CN—CH₂—.

16. The compound of any one of claims 1-15, wherein each of R⁷ and R⁹ is independently hydrogen, alkyl, or —C(O)R^{7a}, wherein said alkyl is unsubstituted or substituted with halogen, cyano, hydroxy, or alkoxy, and wherein R^{7a} is hydrogen, alkyl, or alkoxy; preferably each of R⁷ and R⁹ is independently C₁₋₄alkyl; more preferably each of R⁷ and R⁹ is independently C₁₋₂alkyl.

17. The compound of any one of claims 1-16, wherein R⁷ and R⁹ are each independently hydrogen, methyl, ethyl, methoxymethyl, 2-methoxyethyl, cyanomethyl, hydroxyethyl, hydroxymethyl, methoxycarbonyl, difluoromethyl, provided that at least one of R⁷ and R⁹ is not hydrogen.

18. The compound of any one of claims 1-17, wherein R⁷ is methyl, and R⁹ is methyl; or R⁷ is ethyl, and R⁹ is ethyl; or R⁷ is methyl, and R⁹ is ethyl; or R⁷ is ethyl, and R⁹ is methyl; or R⁷ is methyl, and R⁹ is methoxycarbonyl; or R⁷ is hydrogen, and R⁹ is methyl; or R⁷ is hydrogen, and R⁹ is ethyl.

19. The compound of any one of claims 1-18, wherein X² is N, and X³ is N; or X² is N, and X³ is CH; or X² is CH, and X³ is N; or X² is CH, and X³ is CH; preferably X² is N, and X³ is N.

20. The compound of any one of claims 1-19, wherein X² and X³ are nitrogen, and the 2-position carbon on the piperazine ring is S-configuration, and the 5-position carbon on the piperazine ring is R-configuration

21. The compound of any one of claims 1-20, wherein R⁸ and R¹⁰ are each hydrogen.

22. The compound of any one of claims 1-21, wherein L¹ is a direct bond, —O—, —N(R^{L})—, -alkylene- or —C(O)—, wherein R^{L} is hydrogen or alkyl and wherein said -alkylene- is unsubstituted or substituted with halogen, alkoxy, or heterocyclyl; preferably L¹ is C₁₋₄alkylene, preferably C₁₋₂alkylene; more preferably L¹ is a direct bond, —CH₂—, —CH(CH₃)—, —CH(CH₂CH₃)—, —CH(CHF₂)—, —N(H)—, —N(CH₃)—, —O—, —CH(C(O)—NHCH₂CH₂OCH₃)— or —C(CH₃)₂—; much more preferably L¹ is —CH₂—, —CH(CH₂CH₃)—, or CH(CH₃)—; much more preferably L¹ is CH(CH₃)—.

23. The compound of any one of claims 1-22, wherein Cy¹ is aryl, heterocyclyl, heteroaryl, or cycloalkyl, each of which is unsubstituted or substituted with one, two or three substituents R^{3a}, wherein R^{3a} is selected from deuterium, alkoxy, halogen-substituted alkoxy, alkoxyalkyl-, alkyl, halogen-substituted alkyl, halogen, R^{3b}—SO₂—, cycloalkyl, hydroxyalkyl-, cyano, R^{3b}—C(O)—N(R^{3c})—, cyano-substituted alkyl, N(R^{3b}R^{3c})—C(O)—, R^{3b}—O—C(O)—, or heterocyclyl, said cycloalkyl or heterocyclyl is unsubstituted or substituted with alkoxy, alkyl, halogen, or hydroxy, wherein R^{3b} and R^{3c} are each independently hydrogen or alkyl; preferably R^{3a} is selected from deuterium, fluoro, bromo, chloro, methyl, difluoromethyl, trifluoromethoxy, methoxy, methoxymethyl, trifluoromethyl, methylsulfonyl, difluoro, ethoxy, isopropyl, isopropoxy, difluoromethoxy, cyclopropyl, 2,2-difluorocyclopropyl, 1-fluorocyclopropyl, 2-fluorocyclopropyl, cyclobutyl, 1-hydroxyethyl, 2-hydroxyethyl, ethyl, 1,1-difluoroethyl, cyano, dimethoxy, dichloro, acetamido, 1-methoxyethyl, cyanomethyl, carbamoyl, methoxycarbonyl, dimethylcarbamoyl, (difluoromethoxy)methyl, amino, 1-(difluoromethoxy)ethyl, azetidin-1-yl, 2-methoxypropan-2-yl, 1-methoxycyclopropyl, oxetan-3-yl, 1-methylazetidin-3-yl, 1-hydroxyazetidin-3-yl, 2-cyanoethyl, 2-cyanopropan-2-yl, hydroxymethyl, 3-hydroxypropyl, 2-hydroxypropan-2-yl, or 2-fluoropropan-2-yl.

24. The compound of any one of claims 1-23, wherein Cy¹ is an aryl of from 6 to 14 carbon atoms having a single ring or multiple condensed rings which is unsubstituted or substituted with one, two or three R^{3a}.

25. The compound of claim 24, wherein said aryl is phenyl, naphthyl or anthryl, indanyl, or tetrahydronaphthyl, which is unsubstituted or substituted with one, two or three R^{3a}.

26. The compound of any one of claims 1-23, wherein Cy¹ is a monocyclic 5- to 9-membered heterocyclyl or a bicyclic 7- to 10-membered heterocyclyl which is unsubstituted or substituted with one, two or three R^{3a}.

27. The compound of claim 26, wherein said monocyclic 5- to 9-membered heterocyclyl is tetrahydrofuranyl, tetrahydropyranyl, 1,4-dioxanyl, piperidinyl, piperazinyl, or dihydropyridinyl, each of which is unsubstituted or substituted with one, two or three R^{3a}.

28. The compound of any one of claims 1-23, wherein Cy¹ is a monocyclic 5- to 9-membered heteroaryl or a bicyclic 7- to 10-membered heteroaryl which is unsubstituted or substituted with one, two or three R^{3a}.

29. The compound of claim 28, wherein said monocyclic 5- to 9-membered heteroaryl is pyrazolyl, imidazolyl, pyridinyl, pyrimidinyl, pyrazinyl or pyridazinyl, each of which is unsubstituted or substituted with one, two or three R^{3a}.

30. The compound of claim 29, wherein said monocyclic 5- to 9-membered heteroaryl is 1H-imidazol-2-yl, 1H-imidazol-4-yl, 1H-imidazol-5-yl, pyridin-2-yl, pyridin-3-yl, pyridin-4-yl, pyrimidin-2-yl, pyrimidin-4-yl, pyrimidin-5-yl, 1H-indol-2-yl, 1H-indol-3-yl, 1H-indol-4-yl, 1H-indol-5-yl, 1H-indol-6-yl, 1H-indol-7-yl, pyrazin-2-yl, or pyridazin-4-yl, each of which is unsubstituted or substituted with one, two or three R³.

31. The compound of claim 28, wherein said bicyclic 7- to 10-membered heteroaryl is indolyl, benzo[d]imidazolyl, triazolopyridinyl, imidazopyridinyl, benzooxazolyl, benzo[d]thiazolyl, quinolinyl, isoquinolinyl, naphthyridinyl, dioxinopyridineyl, quinoxalinyl, benzo[d]imidazolyl, imidazo[4,5-b]pyridinyl, thiazolo[5,4-b]pyridinyl, thiazolo[4,5-b]pyridinyl, thieno[2,3-b]pyridinyl, or thieno[3,2-b]pyridinyl, each of which is unsubstituted or substituted with one, two or three R³.

32. The compound of claim 28, wherein said bicyclic 7- to 10-membered heteroaryl is 1H-benzo[d]imidazol-2-yl, 1H-benzo[d]imidazol-4-yl, 1H-benzo[d]imidazol-5-yl, 1H-benzo[d]imidazol-6-yl, 1H-benzo[d]imidazol-7-yl, [1,2,4]triazolo[1,5-a]pyridin-2-yl, [1,2,4]triazolo[1,5-a]pyridin-5-yl, [1,2,4]triazolo[1,5-a]pyridin-6-yl, [1,2,4]triazolo[1,5-a]pyridin-7-yl, [1,2,4]triazolo[1,5-a]pyridin-8-yl, 3H-imidazo[4,5-b]pyridine-2-yl, 3H-imidazo[4,5-b]pyridine-5-yl, 3H-imidazo[4,5-b]pyridine-6-yl, 3H-imidazo[4,5-b]pyridine-7-yl, 1H-imidazo[4,5-b]pyridin-2-yl, 1H-imidazo[4,5-b]pyridin-5-yl, 1H-imidazo[4,5-b]pyridin-6-yl, 1H-imidazo[4,5-b]pyridin-7-yl, benzo[d]oxazol-2-yl, benzo[d]oxazol-4-yl, benzo[d]oxazol-5-yl, benzo[d]oxazol-6-yl, benzo[d]oxazol-7-yl, benzo[d]thiazol-2-yl, benzo[d]thiazol-4-yl, benzo[d]thiazol-5-yl, benzo[d]thiazol-6-yl, benzo[d]thiazol-7-yl, quinolin-2-yl, quinolin-3-yl, quinolin-4-yl, quinolin-5-yl, quinolin-6-yl, quinolin-7-yl, isoquinolin-1-yl, isoquinolin-3-yl, isoquinolin-4-yl, isoquinolin-5-yl, isoquinolin-6-yl, isoquinolin-7-yl, isoquinolin-8-yl, quinoxalin-2-yl, quinoxalin-3-yl, quinoxalin-4-yl, quinoxalin-5-yl, quinoxalin-6-yl, quinoxalin-7-yl, quinoxalin-8-yl, 1,8-naphthyridin-2-yl, 1,8-naphthyridin-3-yl, 1,8-naphthyridin-4-yl, 2,3-dihydro-[1,4]dioxino[2,3-b]pyridine-6-yl, 2,3-dihydro-[1,4]dioxino[2,3-b]pyridine-7-yl, 2,3-dihydro-[1,4]dioxino[2,3-b]pyridine-8-yl, quinoxalin-6-yl-2,3-d2, 1H-indol-2-yl, 1H-benzo[d]imidazol-2-yl, 1-methyl-1H-benzo[d]imidazol-6-yl, 3H-imidazo[4,5-b]pyridin-2-yl, 4,5,6,7-tetrahydro-1H-benzo[d]imidazol-2-yl, 2,3-dihydro-[1,4]dioxino[2,3-b]pyridin-6-yl, thiazolo[5,4-b]pyridin-2-yl, thiazolo[5,4-b]pyridin-5-yl, thiazolo[5,4-b]pyridin-6-yl, thiazolo[5,4-b]pyridin-7-yl, thiazolo[4,5-b]pyridin-2-yl, thiazolo[4,5-b]pyridin-5-yl, thiazolo[4,5-b]pyridin-6-yl, thiazolo[4,5-b]pyridin-7-yl, thieno[2,3-b]pyridin-2-yl, thieno[2,3-b]pyridin-3-yl, thieno[2,3-b]pyridin-4-yl, thieno[2,3-b]pyridin-5-yl, thieno[2,3-b]pyridin-6-yl, thieno[3,2-b]pyridin-2-yl, thieno[3,2-b]pyridin-3-yl, thieno[3,2-b]pyridin-5-yl, thieno[3,2-b]pyridin-6-yl, thieno[3,2-b]pyridin-7-yl, each of which is unsubstituted or substituted with one, two or three R.

33. The compound of claim 28, wherein Cy¹ is quinoxalin-2-yl, quinoxalin-3-yl, quinoxalin-4-yl, quinoxalin-5-yl, quinoxalin-6-yl, quinoxalin-7-yl, quinoxalin-8-yl, preferably quinoxalin-6-yl, which is unsubstituted or substituted with one, two or three R^{3a}, wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, or heterocyclyl, wherein said alkyl moiety in the group alkyl or alkoxy is unsubstituted or substituted with halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl; preferably R^{3a} is selected from deuterium, alkoxy, halogen-substituted alkoxy, alkoxyalkyl-, alkyl, halogen-substituted alkyl, halogen, R^{3b}—SO₂—, cycloalkyl, hydroxyalkyl-, cyano, R^{3b}—C(O)—N(R^{3c})—, cyano-substituted alkyl, N(R^{3b}R^{3c})—C(O)—, N(R^{3b}R^{3c})—, R^{3b}—O—C(O)—, or heterocyclyl, said cycloalkyl or heterocyclyl is unsubstituted or substituted with alkoxy, alkyl, halogen, or hydroxy, wherein R^{3b} and R^{3c} are each independently hydrogen or alkyl; more preferably R^{3a} is selected from deuterium, fluoro, bromo, chloro, methyl, difluoromethyl, trifluoromethoxy, methoxy, methoxymethyl, trifluoromethyl, methylsulfonyl, difluoro, ethoxy, isopropoxy, trifluoromethoxy, difluoromethoxy, cyclopropyl, 1-hydroxyethyl, ethyl, 1,1-difluoroethyl, cyano, dimethoxy, dichloro, cyclopropyl-, acetamido, 1-methoxyethyl, cyanomethyl, carbamoyl, methoxycarbonyl, dimethylcarbamoyl, (difluoromethoxy)methyl, amino, 1-(difluoromethoxy)ethyl, azetidin-1-yl, 2-methoxypropan-2-yl, 1-methoxycyclopropyl, oxetan-3-yl, 1-methylazetidin-3-yl, or 1-hydroxyazetidin-3-yl.

34. The compound of claim 33, wherein Cy¹ is quinoxalin-6-yl, benzo[d]thiazol-5-yl, benzo[d]thiazol-6-yl, thiazolo[5,4-b]pyridin-5-yl, thiazolo[5,4-b]pyridin-6-yl, thiazolo[5,4-b]pyridin-7-yl, thiazolo[4,5-b]pyridin-5-yl, thiazolo[4,5-b]pyridin-6-yl, thiazolo[4,5-b]pyridin-7-yl, thieno[2,3-b]pyridin-6-yl or thieno[3,2-b]pyridin-5-yl, each of which is unsubstituted or substituted with one, two or three R^{3a}, wherein R^{3a} is deuterium, methyl, ethyl, isopropyl, cyanomethyl, 2-cyanoethyl, 2-cyanopropan-2-yl, hydroxymethyl, 2-hydroxyethyl, 1-hydroxyethyl, 3-hydroxypropyl, 2-hydroxypropan-2-yl, difluoromethyl, trifluoromethyl, 2-fluoropropan-2-yl, methoxy, isopropoxy, difluoromethoxy, fluoro, chloro, cyano, amino, cyclopropyl, 2,2-difluorocyclopropyl, 1-fluorocyclopropyl, 2-fluorocyclopropyl, or cyclobutyl.

35. The compound of claim 34, wherein Cy¹ is quinoxalin-6-yl, which is unsubstituted or substituted with one, two or three R^{3a}, wherein R^{3a} is deuterium, methyl, ethyl, isopropyl, cyanomethyl, 2-cyanoethyl, 2-cyanopropan-2-yl, hydroxymethyl, 2-hydroxyethyl, 1-hydroxyethyl, 3-hydroxypropyl, 2-hydroxypropan-2-yl, difluoromethyl, trifluoromethyl, 2-fluoropropan-2-yl, methoxy, isopropoxy, difluoromethoxy, fluoro, chloro, cyano, amino, cyclopropyl, 2,2-difluorocyclopropyl, 1-fluorocyclopropyl, 2-fluorocyclopropyl, or cyclobutyl.

36. The compound of claim 23, wherein Cy1 is
phenyl, 2-(trifluoromethoxy)phenyl, 2-methoxyphenyl, 2-(methoxymethyl)phenyl, 2-(trifluoromethyl)phenyl, 4-fluoro-2-(methoxymethyl)phenyl, 4-fluoro-2-methoxyphenyl, 4-fluoro-2-methylphenyl, 2-bromo-4-fluorophenyl, 4-fluoro-2-(methylsulfonyl)phenyl, 4-methyl-2-(trifluoromethyl)phenyl, 2-chloro-4-fluorophenyl, 2,4-difluorophenyl, 2-ethoxy-4-fluorophenyl, 4-fluoro-2-isopropoxyphenyl, 4-fluoro-2-(trifluoromethoxy)phenyl, 2-(difluoromethoxy)-4-fluorophenyl, 2-(difluoromethyl)-4-fluorophenyl, 2-cyclopropyl-4-fluorophenyl, 4-fluoro-2-(1-hydroxyethyl)phenyl, 4-cyclopropyl-2-methoxyphenyl,2-ethyl-4-fluorophenyl, 4-fluoro-2-(trifluoromethyl)phenyl, 2-methoxy-4-fluorophenyl, 2-(1,1-difluoroethyl)-4-fluorophenyl, 2-cyano-4-fluorophenyl, 4-fluoro-3-(methoxymethyl)phenyl, 3-methyl-2-(trifluoromethyl)phenyl, 4-fluoro-2,6-dimethoxyphenyl, 2,4-difluoro-6-methoxyphenyl, 2,6-dichloro-4-fluorophenyl, 4-cyclopropylphenyl, 4-methoxyphenyl, 4-fluorophenyl, 4-cyclopropyl-phenyl, 4-(trifluoromethyl)phenyl, 4-methylphenyl, 4-(difluoromethyl)phenyl, 4-isopropoxyphenyl, 2-fluoro-4-(trifluoromethyl)phenyl, 4-cyclopropyl-2-fluorophenyl, 3-methoxy-4-(trifluoromethyl)phenyl, 4-fluoro-3-methoxyphenyl, 2,6-difluorophenyl, 4-(trifluoromethoxy)phenyl, 4-acetamidophenyl, 4-fluoro-2-(1-methoxyethyl)phenyl, 2-(cyanomethyl)-4-fluorophenyl, 3,4-difluoro-2-(trifluoromethyl)phenyl, 2-carbamoyl-4-fluorophenyl, 2-methoxycarbonyl-4-fluorophenyl, 2-(dimethylcarbamoyl)-4-fluorophenyl, 2-((difluoromethoxy)methyl)-4-fluorophenyl, 2-(1-(difluoromethoxy)ethyl)-4-fluorophenyl, 2-(azetidin-1-yl)-4-fluorophenyl, 4-fluoro-2-(2-methoxypropan-2-yl)phenyl, 3-(trifluoromethyl)phenyl, 4-fluoro-2-(1-methoxycyclopropyl)phenyl, 4-fluoro-2-(oxetan-2-yl)phenyl, 4-fluoro-2-(1-methylazetidin-3-yl)phenyl, 4-fluoro-2-(1-hydroxyazetidin-3-yl)phenyl, 4-cyclopropyl-2-methoxyphenyl; 2-ethyl-4-fluorophenyl; 2,6-difluoro-4-methoxyphenyl; 2,5-difluoro-4-methoxyphenyl; or naphthalen-2-yl; or
benzo[d][1,3]dioxol-5-yl, 2,2-dimethylbenzo[d][1,3]dioxol-5-yl, 2,2-dimethylbenzo[d][1,3]dioxol-5-yl, 2,3-dihydrobenzo[b][1,4]dioxin-6-yl, 3,3-dimethyl-2,3-dihydrobenzo[b][1,4]dioxin-6-yl, 3,3-difluoro-2,3-dihydrobenzo[b][1,4]dioxin-6-yl, 2,2-difluorobenzo[d][1,3]dioxol-4-yl, 2,2-dimethylbenzo[d][1,3]dioxol-4-yl, 3,4-dihydro-2H-benzo[b][1,4]oxazin-6-yl, or 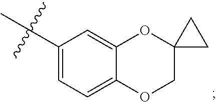 [SMILES: *c1ccc2c(c1)OC1(CC1)CO2]
or
tetrahydrofuran-2-yl, tetrahydrofuran-3-yl, tetrahydro-2H-pyran-2-yl, tetrahydro-2H-pyran-3-yl, tetrahydro-2H-pyran-4-yl, 1,4-dioxan-2-yl, 1,4-dioxan-3-yl, piperidin-2-yl, piperidin-3-yl, piperidin-4-yl, piperazin-2-yl, piperazin-3-yl, 1,2-dihydropyridin-3-yl, 1,2-dihydropyridin-4-yl, 1,2-dihydropyridin-5-yl, or 1,2-dihydropyridin-6-yl; or
chroman-2-yl, chroman-3-yl, or chroman-4-yl; or
1H-pyrazol-3-yl, 1H-pyrazol-4-yl, 1H-pyrazol-5-yl, 1-ethyl-5-(trifluoromethyl)-1H-pyrazol-4-yl; 5-ethyl-1-methyl-1H-pyrazol-4-yl; 1H-imidazol-2-yl, 1H-imidazol-4-yl, 1H-imidazol-5-yl, pyridin-2-yl, pyridin-3-yl, pyridin-4-yl, pyrimidin-2-yl, pyrimidin-4-yl, pyrimidin-5-yl, 1H-indol-2-yl, 1H-indol-3-yl, 1H-indol-4-yl, 1H-indol-5-yl, 1H-indol-6-yl, 1H-indol-7-yl, 3-methoxypyridin-2-yl, 6-isopropoxypyridin-3-yl, 2-methoxypyridin-3-yl, 2-(trifluoromethyl)pyridin-3-yl, 6-(difluoromethoxy)pyridin-3-yl, 3-methoxypyridin-4-yl, 3-(trifluoromethyl)pyridin-4-yl, 5-fluoro-3-(trifluoromethyl)pyridin-2-yl, 5-chloro-1-ethyl-1H-imidazol-2-yl, 1-ethyl-2-(trifluoromethyl)-1H-imidazol-5-yl, 1-ethyl-5-(trifluoromethyl)-1H-pyrazol-4-yl, 5-ethyl-1-methyl-1H-pyrazol-4-yl, 1-methyl-5-(trifluoromethyl)-1H-pyrazol-4-yl, 5-(difluoromethyl)-1-methyl-1H-pyrazol-4-yl, 1-ethyl-3-(trifluoromethyl)-1H-pyrazol-5-yl, 3-chloro-1-ethyl-1H-pyrazol-5-yl, 1-ethyl-4-methyl-1H-pyrazol-5-yl, 5-isopropoxypyridin-2-yl, 6-(trifluoromethyl)pyridin-3-yl, 3,5-difluoropyridin-2-yl, 3,5-difluoropyridin-4-yl, 1-ethyl-4-cyano-1H-pyrazol-3-yl, 5-fluoropyridin-2-yl, pyrazin-2-yl, 3-(trifluoromethyl)pyridazin-4-yl, 6-cyclopropylpyridin-3-yl, 6-cyclobutylpyridin-3-yl, 6-isopropylpyridin-3-yl, 6-cyanopyridin-3-yl, 6-(2-cyanopropan-2-yl)pyridin-3-yl, 6-(2-hydroxypropan-2-yl)pyridin-3-yl, 6-cyclopropyl-2-fluoropyridin-3-yl, 6-methoxypyridin-3-yl, 6-(2-fluoropropan-2-yl)pyridin-3-yl, 5-methoxypyridin-2-yl, 5-cyclopropylpyridin-2-yl, 6-(2,2-difluorocyclopropyl)pyridin-3-yl, 5-(difluoromethyl)pyridin-2-yl, 5-(trifluoromethyl)pyridin-2-yl, 6-(1-fluorocyclopropyl)pyridin-3-yl, 6-(2-fluorocyclopropyl)pyridin-3-yl, or 2-cyclopropylpyrimidin-5-yl; or
1H-benzo[d]imidazol-2-yl, 1H-benzo[d]imidazol-4-yl, 1H-benzo[d]imidazol-5-yl, 1H-benzo[d]imidazol-6-yl, 1H-benzo[d]imidazol-7-yl, [1,2,4]triazolo[1,5-a]pyridin-2-yl, [1,2,4]triazolo[1,5-a]pyridin-5-yl, [1,2,4]triazolo[1,5-a]pyridin-6-yl, [1,2,4]triazolo[1,5-a]pyridin-7-yl, [1,2,4]triazolo[1,5-a]pyridin-8-yl, 3H-imidazo[4,5-b]pyridine-2-yl, 3H-imidazo[4,5-b]pyridine-5-yl, 3H-imidazo[4,5-b]pyridine-6-yl, 3H-imidazo[4,5-b]pyridine-7-yl, 1H-imidazo[4,5-b]pyridin-2-yl, 1H-imidazo[4,5-b]pyridin-5-yl, 1H-imidazo[4,5-b]pyridin-6-yl, 1H-imidazo[4,5-b]pyridin-7-yl, benzo[d]oxazol-2-yl, benzo[d]oxazol-4-yl, benzo[d]oxazol-5-yl, benzo[d]oxazol-6-yl, benzo[d]oxazol-7-yl, benzo[d]thiazol-2-yl, benzo[d]thiazol-4-yl, benzo[d]thiazol-5-yl, benzo[d]thiazol-6-yl, benzo[d]thiazol-7-yl, 2-methylbenzo[d]thiazol-6-yl, 5-fluorobenzo[d]thiazol-6-yl, 5-fluoro-2-methylbenzo[d]thiazol-6-yl, 6-fluoro-2-methylbenzo[d]thiazol-5-yl, 7-fluorobenzo[d]thiazol-6-yl, 7-fluoro-2-methylbenzo[d]thiazol-6-yl, 4-fluorobenzo[d]thiazol-5-yl, quinolin-2-yl, quinolin-3-yl, quinolin-4-yl, quinolin-5-yl, quinolin-6-yl, quinolin-7-yl, isoquinolin-1-yl, isoquinolin-3-yl, isoquinolin-4-yl, isoquinolin-5-yl, isoquinolin-6-yl, isoquinolin-7-yl, isoquinolin-8-yl, quinoxalin-2-yl, quinoxalin-3-yl, quinoxalin-4-yl, quinoxalin-5-yl, quinoxalin-6-yl, quinoxalin-7-yl, quinoxalin-8-yl, 1,8-naphthyridin-2-yl, 1,8-naphthyridin-3-yl, 1,8-naphthyridin-4-yl, 2,3-dihydro-[1,4]dioxino[2,3-b]pyridine-6-yl, 2,3-dihydro-[1,4]dioxino[2,3-b]pyridine-7-yl, 2,3-dihydro-[1,4]dioxino[2,3-b]pyridine-8-yl, 3-methylquinoxalin-6-yl, 3-methoxyquinoxalin-6-yl, quinoxalin-6-yl-2,3-d2,1-ethyl-1H-indol-2-yl, 1-methyl-1H-benzo[d]imidazol-2-yl, 1-ethyl-1H-benzo[d]imidazol-2-yl, 1-propyl-1H-benzo[d]imidazol-2-yl, 1-ethyl-5-(trifluoromethyl)-1H-benzo[d]imidazol-2-yl, 1-ethyl-6-(trifluoromethyl)-1H-benzo[d]imidazol-2-yl, 1-ethyl-7-(trifluoromethyl)-1H-benzo[d]imidazol-2-yl, 1-methyl-1H-benzo[d]imidazol-6-yl, 3-ethyl-3H-imidazo[4,5-b]pyridin-2-yl, 1-ethyl-1H-imidazo[4,5-b]pyridin-2-yl, 3-cyclopropylquinoxalin-6-yl,
3-aminoquinoxalin-6-yl, 3-trifluoromethylquinoxalin-6-yl, 3-bifluoromethylquinoxalin-6-yl, 3-(1,1-bifluoroethyl)quinoxalin-6-yl, 2-deuterium-3-methylquinoxalin-6-yl, 2-deuterium-3-methoxyquinoxalin-6-yl, 3-methyl-5-methoxyquinoxalin-6-yl, 3-methyl-7-methoxyquinoxalin-6-yl, 3-methyl-5-trifluoromethylquinoxalin-6-yl, 3-methyl-7-trifluoromethylquinoxalin-6-yl, 1-ethyl-4,5,6,7-tetrahydro-1H-benzo[d]imidazol-2-yl, 2,3-dihydro-[1,4]dioxino[2,3-b]pyridin-6-yl, 2,3-dimethyl-quinoxalin-6-yl, 3-ethyl-quinoxalin-6-yl, 3-chloro-quinoxalin-6-yl, 4-methoxy-quinoxalin-6-yl, 3-(difluoromethyl)quinoxalin-6-yl, 3-(1,1-difluoroethyl)quinoxalin-6-yl, or 3-cyclopropyl-quinoxalin-6-yl; or cyclobutyl, cyclopentyl, cyclohexyl, 2,3-dihydro-1H-inden-1-yl, 2,3-dihydro-1H-inden-2-yl, 1,2,3,4-tetrahydronaphthalen-1-yl, 1,2,3,4-tetrahydronaphthalen-2-yl, or 6,7,8,9-tetrahydro-5H-benzo[7]annulen-5-yl; or
thiazolo[5,4-b]pyridin-5-yl, 2-methylthiazolo[5,4-b]pyridin-5-yl, thiazolo[4,5-b]pyridin-5-yl, 2-methylthiazolo[4,5-b]pyridin-5-yl, thieno[2,3-b]pyridin-6-yl, 2-methylthieno[2,3-b]pyridin-6-yl, 2-methylthieno[3,2-b]pyridin-5-yl, 2-fluorothieno[3,2-b]pyridin-5-yl, or 2-fluorothieno[2,3-b]pyridin-6-yl.

37. The compound of any one of claims 1-35, wherein the compound is selected from the compounds in Table 1.

38. A pharmaceutical composition comprising one or more compounds of any one of claims 1-37, or a stereoisomer or pharmaceutically acceptable salt thereof, and a pharmaceutically acceptable excipient.

39. A compound, or a stereoisomer or pharmaceutically acceptable salt thereof, said compound is any one of the exemplified compounds.

40. A method of treating a disease, comprising administering to a patient in need of such treatment a therapeutically effective amount of a compound of any one of claims 1-37, wherein the disease is cancer.
